(19) United States
(12) Patent Application Publication
(10) Pub. No.: US 2025/0345370 A1
(43) Pub. Date: Nov. 13, 2025

(54) MICROBIAL LIBERATION OF N-METHYLSEROTONIN FROM ORANGE FIBER

(71) Applicant: Washington University, St. Louis, MO (US)

(72) Inventors: Nathan HAN, St. Louis, MO (US); Jiye CHENG, St. Louis, MO (US); Michael BARRATT, St. Louis, MO (US); Jeffrey GORDON, St. Louis, MO (US)

(73) Assignee: Washington University, St. Louis, MO (US)

(21) Appl. No.: 18/856,702

(22) PCT Filed: Apr. 14, 2023
(86) PCT No.: PCT/US2023/065767

§ 371 Date: Oct. 14, 2024

Related U.S. Application Data
(60) Provisional application No. 63/331,038, filed on Apr. 14, 2022.

(51) Int. Cl. A61K 35/741; A61K 31/4045; A61K 31/555; A61P 1/14; C12N 1/20; C12R 1/01

(52) U.S. Cl. CPC A61K 35/741; A61K 31/4045; A61K 31/555; A61P 1/14; C12N 1/20; C12R 2001/01

(57) ABSTRACT

The present disclosure is directed to compositions and methods for effectively liberating bioactive compounds from fibers for significant host physiological and metabolic benefit.

### CROSS-REFERENCE TO RELATED APPLICATIONS

This application claims priority from U.S. Provisional Application Ser. No. 63/331,038 filed on 14 Apr. 2022, which is incorporated herein by reference in its entirety.

### STATEMENT REGARDING FEDERALLY SPONSORED RESEARCH & DEVELOPMENT

This invention was made with government support under grant number DK070977 awarded by the National Institutes of Health. The government has certain rights in the invention.

### SEQUENCE LISTING

The Sequence Listing, which is a part of the present disclosure, includes a computer-readable form comprising nucleotide and/or amino acid sequences of the present invention (file name “020026-WO-US_2025-07-07_Sequence-Listing-Corrected.xml” created on 7 Jul. 2025; 2,986 bytes). The subject matter of the Sequence Listing is incorporated herein by reference in its entirety.

### FIELD OF THE DISCLOSURE

The field of the disclosure relates generally to mining bioactive compounds (e.g., N-methylserotonin) from natural fiber sources using specialized gut microbes, as well as therapeutic prebiotic, probiotic, or synbiotic compositions and methods thereof.

### BACKGROUND OF THE DISCLOSURE

Identifying the products of metabolism of dietary components by members of human gut communities and determining how these products mediate microbe-microbe and microbe-host interactions holds the promise of generating new approaches for modulating host functions in ways that improve health status. Dietary fibers exemplify this point. Fibers are chemically complex; they include but are not limited to structurally diverse polysaccharide components, proteins and lipids. The association between increased consumption of dietary fiber and improved health status is widely recognized. Some of the underlying mediators and mechanisms are well known. For example, short-chain fatty acids produced by microbial metabolism of otherwise indigestible plant polysaccharides have been linked to beneficial health outcomes. The gut microbiota affects the bioavailability of (poly) phenolic compounds contained in dietary fiber by metabolizing them to smaller bioactive products. In addition to these observations about fiber, there is a rapidly expanding knowledge base of how the products of microbial community metabolism and microbial-host co-metabolism affect human biology in healthy and disease states.

Population growth, the existential threat posed by climate change, and associated challenges to environmental sustainability have focused attention on the design of eco-friendly food systems; this includes management of the massive amount of inorganic as well as organic ‘waste’ generated during the food manufacture. Fibers are well represented in many of these manufacturing streams; for example, in the peels, rinds and seeds discarded from different fruits and vegetables. The composition of the fibers present in these byproduct streams reflect their differing sources as well as the various mechanical, physical and chemical steps applied during food processing.

Fibers from these manufacturing streams represent a potentially enormous biorepository of unknown or largely uncharacterized natural molecular entities having health promoting effects. Moreover, the biochemical versatility of microbes present in the human gut microbiota provide a resource for liberating these compounds. For example, N-methylserotonin is a tryptamine alkaloid found in commercial food-grade preparations of orange fiber that are generated as a byproduct (waste stream) of the juice making process. However, N-methylserotonin is physically entrapped within orange fiber. Consequently, it cannot be easily extracted (such as with water, methanol, acetonitrile) and is thus not ‘bioavailable’ in its native form.

Accordingly, there is a need for compositions and methods for effectively liberating bioactive compounds from fibers for significant host physiological and metabolic benefit.

### BRIEF DESCRIPTION OF THE DISCLOSURE

The present disclosure illustrates embodiments for harnessing microbial mining capacity to identify chemical entities naturally contained within fibers emanating from manufacturing streams, defining their effects on host physiology, characterizing the mechanism underlying microbial mining, and translating preclinical model results to humans. More specifically, the present disclosure describes prebiotic (orange fiber alone) compositions and synbiotic (orange fiber plus a specific gut microbial strain capable of mining N-methylserotonin from orange fiber, e.g. B. ovatus TSDC17.2-1.1) compositions. When combined, the orange fiber plus the specific gut microbial strain unexpectedly liberates pharmacologically active levels of N-methylserotonin from the fiber into the gut of a human being or animal. This novel discovery has a number of therapeutic applications, including but not limited to irritable bowel syndrome treatment and potentially aspects of metabolic health/glucose homeostasis. Administration of the synbiotic enables the benefits of orange fiber-derived N-methylserotonin to be realized in subjects whose microbiomes otherwise lack the requisite expressed enzymes for mining this compound from orange fiber.

Gnotobiotic mice colonized with defined consortia of cultured human gut bacterial strains were previously used to characterize the effects of adding 34 different dietary fiber preparations to a diet high in saturated fats and low in fruits and vegetables (abbreviated HiSF-LoFV). This diet was formulated based on the NHANES database of diet consumption patterns by humans living in the USA; ‘high’ and ‘low’ were defined as levels in the upper and lower tertiles of the diets captured in this database. These mice were used to characterize mechanisms by which members compete or cooperate in utilizing specific glycan structures present in these fiber preparations. Germ-free mice plus gnotobiotic mice are herein used and colonized with defined consortia of human gut bacterial taxa that were fed this HiSF-LoFV diet with or without an orange fiber byproduct of juice manufacture. The results revealed microbe-dependent release of N-methylserotonin from the fiber preparation. The effects of N-methylserotonin on host metabolism, and gene expression in the intestine and liver, were characterized by adding this compound to drinking water consumed by germ-free animals. Mechanisms underlying N-methylserotonin release were delineated in vitro, initially with 49 phylogenetically diverse human gut bacterial strains, and then by performing functional genomic analysis under different media conditions using 12 different strains of Bacteroides ovatus, a prominent miner in vivo. Finding that B. ovatus mining activity was regulated by addition or subtraction of a single component (hemin or ferric chloride heme, which is an iron-containing porphyrin) from one of the media tested led to the unexpected discovery that strain-specific expression of genes involved in metabolism of pectic glycans in the fiber preparation correlated with liberation of N-methylserotonin. In a test of a translatability to humans, orange fiber- and control pea fiber-containing snack food prototypes were administered to adult female dizygotic twins in two open-label, single group assignment studies. Levels of N-methylserotonin in feces exhibited a dose-dependent relationship with changes in the representation of bacterial genes encoding glycoside hydrolases and polysaccharide lyases that break down pectic glycans. This approach is generally useful for identifying components of fibers whose liberation under normal physiological conditions requires microbial assistance, yet whose biological/pharmacological activities are not dependent on further microbial biotransformation.

In one aspect, the present disclosure is directed to a synbiotic composition comprising at least one type of plant fiber and at least one microbial strain. In some embodiments, the at least one type of plant fiber comprises a Rutaceae family plant fiber, the Rutaceae family plant fiber comprises citrus fiber, the citrus fiber comprises orange fiber, the at least one microbial strain comprises at least one bacterial strain, the at least one bacterial strain is selected from Bacteroides, Parabacteroides, Collinsella, and combinations thereof, the at least one bacterial strain comprises at least one strain of Bacteroides ovatus, Bacteroides finegoldii, Parabacteroides distasonis, Collinsella aerofaciens, and combinations thereof, the at least one bacterial strain comprises Bacteroides ovatus TSDC 17.2, further comprising an iron-containing porphyrin, and/or the iron-containing porphyrin is hemin.

In another aspect, the present disclosure is directed to a method for locally delivering a bioactive compound to a subject in need thereof, the method comprising administering to the subject a therapeutically effective amount of a synbiotic composition comprising at least one type of plant fiber and at least one microbial strain. In some embodiments, the at least one microbial strain is a source of at least one CAZyme, the at least one CAZyme is selected from PL9, GH5_37, GH5_8, GH59, GH30_5, GH26, GH5_4, GH25, GH13_31, GH123, GH13_19, GH13_28, and combinations thereof, the at least one CAZyme comprises PL9, and/or the bioactive compound is N-methylserotonin.

In yet another aspect, the present disclosure is directed to a method for increasing liver glycogen, increasing tissue glutamate levels, reducing adiposity, reducing high fat diet induced obesity, increasing fatty acid metabolism, decreasing gastrointestinal transit time, colonic motility, and/or treating irritable bowel syndrome in a subject in need thereof, the method comprising administering to the subject a therapeutically effective amount of a synbiotic composition comprising at least one type of plant fiber and at least one microbial strain.

In yet another aspect, the present disclosure is directed to a prebiotic composition comprising an iron-containing porphyrin and at least one type of plant fiber. In some embodiments, the iron-containing porphyrin is hemin, the at least one type of plant fiber comprises a Rutaceae family plant fiber, the Rutaceae family plant fiber comprises citrus fiber, and/or the citrus fiber comprises orange fiber.

In yet another aspect, the present disclosure is directed to a method for locally delivering a bioactive compound to a subject in need thereof, the method comprising administering to the subject a therapeutically effective amount of a prebiotic composition comprising an iron-containing porphyrin and at least one type of plant fiber. In some embodiments, the bioactive compound is N-methylserotonin.

In yet another aspect, the present disclosure is directed to a probiotic composition comprising an iron-containing porphyrin and at least one microbial strain. In some embodiments, the iron-containing porphyrin is hemin, the at least one microbial strain comprises at least one bacterial strain, the at least one bacterial strain is selected from Bacteroides, Parabacteroides, Collinsella, and combinations thereof, the at least one bacterial strain comprises at least one strain of Bacteroides ovatus, Bacteroides finegoldii, Parabacteroides distasonis, Collinsella aerofaciens, and combinations thereof, and/or the at least one bacterial strain comprises Bacteroides ovatus TSDC 17.2.

In yet another aspect, the present disclosure is directed to a method for locally delivering a bioactive compound to a subject in need thereof, the method comprising administering to the subject a therapeutically effective amount of a probiotic composition comprising an iron-containing porphyrin and at least one microbial strain. In some embodiments, the at least one microbial strain is a source of at least one CAZyme, the at least one CAZyme is selected from PL9, GH5_37, GH5_8, GH59, GH30_5, GH26, GH5_4, GH25, GH13_31, GH123, GH13_19, GH13_28, and combinations thereof, the at least one CAZyme comprises PL9, and/or the bioactive compound is N-methylserotonin.

### BRIEF DESCRIPTION OF THE DRAWINGS

The embodiments described herein may be better understood by referring to the following description in conjunction with the accompanying drawings.

FIG. 1(A-C) is an exemplary embodiment of colonization and orange fiber-dependent accumulation of N-methylserotonin in the intestines of gnotobiotic mice in accordance with the present disclosure. FIG. 1A shows cecal contents harvested from germ-free or colonized mice fed either an unsupplemented high saturated fat/low fruits and vegetable (HiSF-LoFV) diet or the HiSF-LoFV diet supplemented with 10% orange fiber, were analyzed by LC-Qtof-MS. The analyte with an m/z of 191.1180 was only found in colonized animals consuming the orange fiber supplemented diet. Chromatograms representative of five biological replicates for each treatment group are shown. FIG. 1B shows collision-induced dissociation mass spectra of an N-methylserotonin standard (upper portion of panel) and cecal extracts (lower portion of panel) obtained by LC-Qtof-MS/MS. FIG. 1C shows levels of N-methylserotonin released after a 72 h incubation of each of 14 bacterial strains with orange fiber in TYG medium. Mean values±SD per 50 mg of orange fiber are shown. See also Table S1, Table S2, and Table S4.

FIG. 2(A-H) is an exemplary embodiment of effects of orally administered N-methylserotonin in germ-free mice in accordance with the present disclosure. FIG. 2A shows experimental design. Groups of adult germ-free mice consumed the HiSF-LoFV diet ad libitum. Animals received one of two doses (1 mg/kg/day and 50 mg/kg/day) of N-methylserotonin in their drinking water for 21 days. FIG. 2B shows percent change in body weight between experimental days 1 and 21. FIG. 2C shows epididymal fat pad weight at the time of euthanasia. FIGS. 2D, 2E, 2F, and 2G show metabolites related to glycogen biosynthesis measured in the liver at euthanasia. FIG. 2H shows transit time through the gastrointestinal tract of germ-free mice measured on day 17 (4-5 mice/treatment group). Mean values±SD are shown in panels B-H. Filled circles indicate values for individual animals. *, p<0.05; **, p<0.01; ***, p<0.001; ****, p<0.0001 (one-way ANOVA). Colors used to denote treatment groups in panels B-H are keyed to the colors employed in panel A. Each dot represents results from a single animal. See also FIG. 6 and Table S3.

FIG. 3(A-G) is an exemplary embodiment of specificity and host physiologic effects of N-methylserotonin release from the orange fiber-supplemented HiSF-LoFV diet by human gut bacterial strains in vivo in accordance with the present disclosure. FIG. 3A shows experimental design. FIG. 3B shows composition of 14-, 10- and 4-member bacterial consortia used to colonize mice. FIG. 3C shows absolute abundances of organisms comprising each consortium as defined by shotgun sequencing of DNA isolated from fecal samples collected on experimental day 21. FIG. 3D shows N-methylserotonin levels in feces obtained on experimental day 21. FIG. 3E shows percent change in body weight between experimental days 1 and 21. FIG. 3F shows epididymal fat pad weight expressed as a percentage of body weight. FIG. 3G shows gastrointestinal transit time. Mean±SD values are shown in panels C-G. *, p<0.05; **, p<0.01; *, p<0.001, ****, p<0.0001 (one-way ANOVA). Colors used in panels C-G denote treatment groups and are keyed to match the colors employed in FIG. 3B. See also Table S5.

FIG. 4(A-C) is an exemplary embodiment of in vitro B. ovatus strain-specific N-methylserotonin ‘mining’ phenotypes and identification of candidate genes involved in its release from orange fiber in accordance with the present disclosure. FIG. 4A and FIG. 4B show in vitro release of N-methylserotonin after a 72 h incubation of each of 12 bacterial strains with orange fiber in TYG medium containing hemin (FIG. 4A) or lacking hemin (FIG. 4B). Mean values±SD per 50 mg orange fiber for triplicate incubations are shown. FIG. 4C shows selection criteria for identifying genes designated as candidate members of the N-methylserotonin mining apparatus of B. ovatus TSDC 17.2 based on their patterns of expression in mining permissive and non-permissive in vitro conditions. All comparisons made are between incubations with or without orange fiber (OF) for 72 h using the indicated culture media and B. ovatus strains (TSDC 17.2 or 115). The log₂ fold differences in expression of the nine genes listed, in the presence or absence of OF in TYG medium containing hemin, are statistically significant (FDR corrected p<0.05; Benjamin and Hochberg). For other conditions: “-” denotes p>0.05 while “N/A” indicates the absence of an ortholog in the genome of strain 115. See also FIG. 7, Table S4, Table S6 and Table S7.

FIG. 5(A-B) is an exemplary embodiment of dose-dependent and fiber-dependent accumulation of N-methylserotonin of adult dizygotic twin pairs consuming fiber snack food prototypes in accordance with the present disclosure. FIG. 5A shows LC-QqQ-MS based measurements of fecal N-methylserotonin levels in members of twin pairs consuming the indicated fiber snack prototypes as a function of the number of snacks consumed per day. Each dot represents data for a single participant. Mean values±SD are shown. **, p<0.001; Friedman's test with Dunn's multiple comparison. FIG. 5B shows Spearman correlation analysis performed between the abundances of all CAZyme genes and levels of N-methylserotonin in fecal samples collected from participants at the end of week 1 (unrestricted diet, no fiber snacks) and at the end of week 5 (unrestricted diet supplemented with 3 orange fiber snacks/day). The heatmap and bar plot display loge-fold changes in the abundances of GH and PL genes and levels of N-methylserotonin. Shown are 12 CAZymes whose abundances in the microbiome were significantly correlated with levels of N-methylserotonin at weeks 1 and 5. Each column in the heatmap and each bar in the bar graph represent the response of an individual study participant. Hierarchical clustering (Euclidean distances) was used to group participants and CAZymes with similar responses to consumption of orange fiber snacks. Participant code: TPO1.01=twin pair 1, co-twin 1. The circles on the right side of the heat map indicate the FDR-corrected statistical significance of the Spearman rho correlation; q<0.1, *q<0.05, **q<0.01. n=22 participants, n=44 fecal samples analyzed. See also Table S8.

FIG. 6 is an exemplary embodiment of over-representation analysis of GO Biological Process terms in the set of genes differentially expressed in the livers of germ-free mice in response to orally administered N-methylserotonin in accordance with the present disclosure. Related to FIG. 2(A-H) and Table S3. GO terms are ranked by gene ratio with a p-value cutoff of 0.05.

FIG. 7 is an exemplary embodiment of HTCS_Rgu-2 regulon analysis in three B. ovatus strains in accordance with the present disclosure. Related to FIG. 4(A-C) and Table S7. Predicted HTCS Rgu-2 binding sites (PUL number and the ID of their component genes are based on B. ovatus TSDC 17.2 and described in Table S7). Predicted members of the HTCS Rgu-2 regulon are indicated by the solid line on top. Sequence logo shows the consensus for identified HTCS Rgu-2 binding sites.

### DETAILED DESCRIPTION OF THE DISCLOSURE

Plant fibers in byproduct streams produced by non-harsh food processing methods represent biorepositories of diverse naturally-occurring physiologically-active biomolecules. To demonstrate one approach for their characterization, mass-spectrometry of intestinal contents from gnotobiotic mice, plus in vitro studies, revealed liberation of N-methylserotonin from orange fibers by human gut microbiota members including Bacteroides ovatus. Functional genomic analyses of B. ovatus strains grown under permissive and non-permissive N-methylserotonin ‘mining’ conditions revealed members of polysaccharide utilization loci that target pectins whose expression correlate with strain-specific liberation of this compound. N-methylserotonin, orally-administered to germfree mice, reduced adiposity, altered liver glycogenesis, shortened gut transit time, and changed expression of genes that regulate circadian rhythm in liver and colon. In human studies, dose-dependent, orange fiber-specific fecal accumulation of N-methylserotonin positively correlated with levels of microbiome genes encoding enzymes that digest pectic glycans. Identifying this type of microbial mining activity has potential therapeutic implications.

According to the present disclosure, when orange fiber preparations are exposed to specific human gut bacteria (in vitro and in vivo), the actions of specific enzymes encoded by this select group of microbes are able to release the entrapped N-methylserotonin from the orange fiber matrix. This produces a soluble/free from of N-methylserotonin that is bioavailable (without further microbial biotransformation) at pharmacologically relevant levels-demonstrated both in the mouse gut, and in the feces of participants in a human study of diet supplementation with orange fiber snacks.

Using a germ free mouse model, we have shown that administration of N-methylserotonin in the drinking water, at concentrations comparable to those ingested by supplementing the diet with an orange fiber preparation, produces beneficial effects on host metabolism and gene expression in the intestine and liver, plus a significant reduction in gut transit time with potential therapeutic implications (e.g. an approach for treatment of certain forms of irritable bowel syndrome).

The present disclosure describes species and strain-level specificity of human gut bacteria that are able to release (‘mine’)N-methylserotonin from orange fiber. Among the cultured, sequenced bacterial strains tested in vitro, several were able to mine N-methylserotonin from orange fiber at low levels, however few possessed strong releasing/mining activity: Bacteroides ovatus TSDC17.2-1.1, Parabacteroides distasonis TSDC17.2-1.1, Collinsella aerofaciens TSDC17.2-1.1, and Bacteroides finegoldii TSDC17.2-1.1. A consortium of those 4 strains, when introduced into germ free mice fed an orange fiber supplemented diet, was able to release N-methylserotonin from the orange fiber into the gut luminal contents of recipient mice. RNA-Seq analysis of gene expression in a ‘strong’ versus ‘weak’ Bacteroides ovatus ‘mining’ strain incubated in the presence or absence of orange fiber in vitro, revealed a set of glycoside hydrolase and polysaccharide lyase genes whose expression were associated with release of N-methylserotonin.

Moreover, the known/predicted substrate specificities of the encoded enzymes were consistent with the prominent representation of pectic polysaccharides present in orange fiber, suggesting that cleavage of these polysaccharides is a prerequisite for release of N-methylserotonin. Further, a study of a small cohort of adult female dizygotic twins who supplemented their normal diets with an escalating dose of a snack food prototype containing orange fiber over a period of 5 weeks disclosed a dose-dependent, orange-fiber specific accumulation of N-methylserotonin in their feces. The orange fiber preparation and its releasable N-methylserotonin are thus be viewed as a natural analog of oral polysaccharide-based drug delivery systems.

### EXPERIMENTAL MODEL

Gnotobiotic mice. Experiments involving gnotobiotic mice were performed using protocols approved by Washington University Animal Studies Committee. Ten-week-old male germ-free C57BL/6J animals were housed in plastic flexible film gnotobiotic isolators (Class Biologically Clean) at 23° C. under a strict 12-hour light cycle (lights on a 0600 h, off at 1800 h).

Germ-free animals were weaned onto an autoclaved, low-fat, plant polysaccharide-rich chow (catalog number 2018S, Envigo) administered ad libitum. Four days prior to colonization, mice were switched to a diet formulation containing ingredients that in aggregate represented the upper tertile of saturated fat consumption and the lower tertile of fruits and vegetable consumption of USA diets as reported in the National Health and Nutrition Examination Survey (NHANES) database. Pelleted unsupplemented HiSF-LoFV diet and the diets supplemented with 10% (w/w) orange fiber (CitriFi 100; Fiber Star) or 10% (w/w) pea fiber (EF 100; Rettenmaiers) were vacuumed packed in plastic bags and subsequently sterilized by gamma irradiation (20-50 kilograys, Steris, Mentor, OH). Sterility was confirmed by culturing the material under aerobic and anaerobic (atmosphere, 75% N2, 20% CO2, 5% H2) conditions at 37° C. in TYG medium.

The bacterial strains used to colonize mice had been cultured from a fecal sample obtained from a lean co-twin in an obesity-discordant twin pair (TSDC 17). Equivalent numbers of bacterial cells (based on OD₆₀₀ measurements) in monocultures (grown in TYG medium under anaerobic conditions to stationary-phase) were pooled to create gavage mixtures. A total of 200 μL of each pool, consisting of all 14 strains, the four strains identified as capable of releasing N-methylserotonin from orange fiber in vitro (B. ovatus, P. distasonis, C. aerofaciens, B. finegoldii), or a mixture of the other 10 strains, were introduced into mice using a plastic-tipped oral gavage needle (Fisher).

Animals were maintained in separate gnotobiotic isolators each dedicated to mice colonized with the same bacterial consortium (n=5 animals/cage). Cages contained autoclaved paper ‘shepherd shacks’ to facilitate their natural nesting behaviors and to provide environmental enrichment. Pre-colonization fecal samples were collected to verify the germ-free status of the mice using both culture and culture-independent assays.

For experiments involving administration of N-methylserotonin to germ-free mice, a stock solution of the compound (100 mg/mL, Santa Cruz Biotechnologies) was prepared in sterile water and filter sterilized (0.2 gm pore size; Nalgene). The outer surface of tubes containing the stock solution was sterilized with Clidox (Pharmacal) and the tubes were introduced into gnotobiotic isolators using standard procedures. The stock solution was then diluted in darkened glass water bottles (Ancare) in order to administer doses of 1 mg/kg/day or 50 mg/kg/day (based on an experimentally determined average consumption of 5 mL of water/day/mouse). Every four days, bottles were replaced with new ones containing fresh N-methylserotonin. Each of the three arms of the experiment, including the control arm where unsupplemented drinking water was administered, consisted of 5 mice. However, in case of the higher dose treatment group, one animal died within the first week without any preceding behavioral changes or signs of illness, or decipherable underlying cause.

Fecal samples and body weights were collected weekly, while food and water intake were monitored daily by comparing pellet mass in the food hopper and the volume of water in water bottles at the beginning and end of a 24 h period and dividing these values by the number of mice per cage. All animals were euthanized between 0830 h and 0930 h without prior fasting. Luminal contents from the proximal and distal halves of the small intestine, the cecum and the colon, host tissues (liver, epididymal fat pads, gastrocnemius muscle, the distal quarter of the small intestine (ileum), cecum and entire colon) plus serum were collected, flash frozen in liquid nitrogen and stored at −80° C. prior to analyses.

Human studies with pea and orange fiber snack prototypes. Two separate open-label, single group assignment studies were performed involving members of the Missouri Adolescent Female Twin Study (MOAFTS) cohort who were age 31-45 years at the time of enrollment. The first study with the pea fiber snack was performed between April and August 2017, while the second study with the orange fiber snack was conducted between August and December 2017. All participants provided written informed consent and the studies were approved by the Washington University Institutional Review Board (IRB ID #201611122). (ClinicalTrials.gov NCT03078283).

The design of the two studies were identical except for the fiber snack supplement used and the number of participants in each study. Individuals who were pregnant or trying to get pregnant, had inflammatory bowel disease, gastrointestinal cancer, hepatitis, HIV, renal failure, or allergies to dairy, eggs, fish, crustacean shellfish, tree nuts, sesame seeds, peanuts, wheat, gluten, soybeans, celery, or mustard were excluded from the study. In Study 1, four twin pairs were concordant for obesity (BMI>30 kg/m2) while five pairs were discordant with one member being obese and the other non-obese (n=18 participants, 36.6-2.9 years (mean±SD); Table S8B). Study 2 involved 24 participants: 12 dizygotic twin pairs [37±2.9 years (mean±SD)], nine of whom had participated in the pea fiber study; for these nine pairs, the interval between cessation of pea fiber snack consumption and initiation of orange fiber consumption ranged from 50 to 106 days [84+26 days (mean±SD)]. Participants consumed their normal, unrestricted diet for the first two weeks of the study (pre-intervention phase). At the beginning of week three, they supplemented their diets with one 35 g fiber snack serving a day for one week, then two 35 g snack servings a day the following week, and thereafter, three 35 g snacks per day for four weeks (weeks 5-8) at breakfast, lunch and dinner. No attempt was made to adjust the diets of participants other than supplementation with the fiber snack. Snack prototypes were manufactured by Mondeldz International, Inc. (see Table S8A for their composition), which participants received in weekly shipments from the study center. The pea fiber snacks were in the form of rotary biscuits (6.7 g total fiber/35 g snack) or extruded bars (8.1 g fiber/35 g snack) with participants having the option to alternate between them. The orange fiber snacks were all in the form of extruded bars (10.2 g total fiber/35 g snack). Compliance was monitored throughout by the study coordinator through weekly phone calls. The primary outcomes for each study were the effects of the respective prototypes on gut microbial community structure and function.

Fecal samples were collected by participants in small medically approved collection containers. Each fecal sample was frozen immediately at −20° C. and temporarily stored in dedicated freezers provided to participants at the beginning of the study. Within 12-48 hours after collection, all samples were shipped, via overnight delivery, in an insulated container containing frozen gel packs, to a biospecimen repository located in Washington University in St. Louis and overseen by one of the authors (A.C.H.). Once received, samples were stored at −80° C. until processing for LC-QqQ-MS analysis of N-methylserotonin levels and culture-independent characterization of ASV and CAZyme gene abundances.

Measurement of fecal N-methylserotonin levels—Each fecal sample was homogenized with a porcelain mortar (4 L) and pestle while submerged in liquid nitrogen; multiple 500 mg aliquots of the pulverized frozen material were stored at −80° C. N-methylserotonin was quantified using the same protocol that was employed for mouse fecal samples (as described herein elsewhere).

Shotgun sequencing of fecal DNA and quantification of CAZyme gene abundances-DNA was purified from fecal samples that had been collected at the t=1 week and 5-week time points from study participants. Sequencing libraries were generated from each purified fecal DNA sample and sequenced [Illumina NextSeq 550 and HiSeq 3000 instruments; 10.7±0.6×10⁶ (mean±SD) and 6.9±1.1×10⁶ (mean±SD) 150 nt paired-end reads/sample). Host-filtered reads were assembled and annotated using prokka (Seemann, 2014) and counts for each open reading frame (ORF) were generated by mapping paired-end reads from each sample to its assembled DNA contigs. Alignments were processed to generate count data (featureCounts; Subread v. 1.5.3 package) for each ORF in each sample and normalized (TPM).

ORFs identified in each fecal sample were used as the starting point for CAZyme annotation. Aggregating abundance data for each sample enabled the generation of CAZyme gene family/subfamily abundance tables. (The abundances of GH and PL genes annotated with multiple CAZyme families/subfamilies were propagated to each individual family/subfamily member, and abundances were then summed across all corresponding CAZyme families within each fecal sample).

16S rDNA amplicon sequencing and identification of ASVs—PCR was performed using purified fecal DNA and barcoded primers directed against variable region 4 of the bacterial 16S rRNA gene. PCR amplification was performed as described in a previous publication; amplicons with sample-specific barcodes were quantified, pooled and sequenced (Illumina MiSeq instrument, paired-end 250 nucleotide reads). Paired-end reads were demultiplexed, trimmed to 200 nucleotides, merged, and chimeras were removed (version 1.13.0 of the DADA2 pipeline). Amplicon sequence variants (ASVs) were aligned against GreenGenes 2016 (v. 13.8) to 97% sequence identity, followed by taxonomic and species assignment [RDP 16 (release 11.5) and SILVA (v. 128)]. The resulting ASV table was filtered to only include those ASVs with >0.1% relative abundance in at least five fecal samples, and then rarefied to 15,000 reads/sample.

### Methods

Measurement of gastrointestinal transit times using non-absorbable red carmine dye. This protocol was adapted from a previously method. Carmine red (Sigma-Aldrich) was prepared as a 6% (w/v) solution in 0.5% methylcellulose (Sigma-Aldrich) and autoclaved prior to import into isolator. Seventeen days after initiation of N-methylserotonin treatment, 200 μL of the carmine red solution were gavaged into each germ-free mouse between 0800 and 0815 h. Feces were collected every 15 minutes and streaked across a sterile white napkin to assay for the presence of the carmine red dye. The time from oral gavage to initial appearance of carmine red in the feces was recorded as the total intestinal transit time for that animal.

Absolute abundances of community members. Short-read community profiling by sequencing (COPRO-Seq) was used to define the absolute abundances of bacterial taxa in fecal samples from colonized mice. For absolute abundance determination, 22.1×10⁶ million Agrobacterium radiobacter DSM 30147 cells and 6.6×10⁶ Alicyclobacillus acidiphilus DSM 14558 cells were added to each frozen fecal pellet. DNA was isolated from the pellets by adding 500 μL of extraction buffer [200 mM Tris (pH 8), 200 mM NaCl, 20 mM EDTA], 210 mL of 20% SDS, and 500 mL of 0.1 mm diameter zirconia beads, followed by treatment with a BioSpec bead beater for 4 minutes, addition of 500 μL phenol:chloroform:isoamyl alcohol (25:24:1), and precipitation of nucleic acids with isopropanol. Libraries were prepared using the Nextera DNA Library Prep Kit (Illumina) and combinations of custom barcoded primers. Multiplex sequencing of the libraries was performed using an Illumina Hi-Seq instrument (paired end 75 nucleotide reads; 2.65×10⁶±1.5×10⁵ reads/sample). Reads were mapped onto the sequenced genomes of consortium members using an analytic pipeline described in previous publication. Absolute abundances, expressed as genome equivalents per gram of material, was calculated for each community member by multiplying the normalized counts of that member with the abundances of the spike-in (number of cells per normalized count) and dividing by the measured weight of the fecal sample.

RNA-Seq of liver and colonic tissue. Frozen tissue was broken into small pieces and ground into a very fine powder under liquid nitrogen using a mortar and pestle. A 25 mg aliquot of powdered tissue was then aliquoted into shearing matrix F (MP Bio) pre-chilled in liquid nitrogen; 0.5 mL of buffer LBP (Takara) was added immediately and the mixture was placed on a 4° C. cold block. Samples were then disrupted (Biospec bead beater; 2 minutes). The remaining steps in the RNA isolation procedure were performed using a Takara Nucleospin RNA Plus kit. After verifying that all purified RNAs had an RNA integrity number (RIN) greater than 8.5 (Agilent RNA Pico), a 1 Ong aliquot of each sample was used to generate a cDNA library (Illumina TruSeq Stranded Total RNA). Libraries were sequenced using an Illumina Hi-Seq instrument (paired end 75 nucleotide reads; 1.43×10⁷±3.74×10⁶ reads/liver sample, and 3.27×10⁷±1.23×10⁶ reads/colon sample). Reads were aligned to the Mus musculus GRCm39 genome assembly with STAR version 2.7.0d. Gene count data were generated from the number of uniquely aligned reads (featureCounts Subread version 1.6.2a). The R package DESEQ2 was used to perform differential gene expression analysis; results were filtered based on an adjusted Benjamini and Hochberg FDR p-value<0.05. Gene set enrichment analysis was carried out using ClusterProfiler with an adjusted p-value cut-off of <0.05 and minimum gene-set size of 3; over-representation was carried out using a loge fold-change cut-off of >1.

In vitro screening of bacterial strains for N-methylserotonin releasing activity. A given bacterial strain was grown in monoculture at 37° C. in TYG medium in an anaerobic chamber (atmosphere; 75% N2, 20% CO2 and 5% H2) to stationary phase. An aliquot was then added to 10 mL of fresh TYG medium with or without 50 mg of orange fiber that had been sterilized by gamma irradiation (30-50 KGy); the mixture was incubated under anaerobic conditions without shaking for 72 hours. A 200 μL aliquot was then removed for targeted LC-QqQ-MS measurement of N-methylserotonin levels; another aliquot was used to define the number of colony-forming units so that levels of the analyte were expressed per 10⁶ cells. An identical protocol was used to compare the amount of N-methylserotonin released when two other rich media, MEGA medium 2.0 and Wilkins-Chalgren anaerobe broth (Thermo-Fisher), were used in lieu of TYG. All incubations were performed in triplicate for each condition.

Experiments to determine whether N-methylserotonin is synthesized de novo by B. ovatus were carried out in 10 mL TYG with or without supplementation with tryptophan, tryptamine, serotonin, dimethylserotonin, trimethylserotonin, methyltryptamine, or S-adenosyl methionine (final concentrations; 5 mg/mL; all from Sigma). Experiments seeking to test the capacity of all 14 bacterial strains introduced into mice to degrade N-methylserotonin in vitro were carried out using 10 mL TYG and 50 ng N-methylserotonin, with samples collected every 24 hours. Assays were performed in triplicate for each condition, using the protocol described herein.

Experiments seeking to test the necessity of having live bacteria to extract N-methylserotonin were carried out by first incubating monocultures of B. ovatus, B. finegoldii, P. distasonis and C. aerofaciens in 10 mL TYG medium at 37° C. under anaerobic conditions to stationary phase. The stationary phase culture was then treated at 70° C. for 1 hour. Cells were recovered by centrifugation (6,000×g for 15 minutes at 4° C.) and the pellet was added to 10 mL of TYG medium containing 5 mg/mL of orange fiber.

Experiments using conditioned media were carried out by taking monocultures of B. ovatus, B. finegoldii, P. distasonis, and C. aerofaciens that had been grown to stationary phase in TYG under anaerobic conditions, centrifuging the culture for 15 minutes at 6,000×g at 4° C. to remove bacterial cells and adding 10 mL of the conditioned medium to 50 mg orange fiber.

Experiments using bacterial lysates were carried out by bead-beating of bacterial cells, collected by centrifugation from 10 mL stationary phase TYG cultures for 4 minutes at room temperature; 500 μL of the resulting lysate was added to 10 mL of a solution containing 5 mg orange fiber/mL TYG medium. To ensure sterility in these experiments, aliquots of the heat-treated cells, centrifuged conditioned media, or bacterial lysate were cultured in TYG medium for 7 days and subsequently plated on TYG-agar; the results confirmed the absence of colony forming units. Assays were performed in triplicate for each experimental condition.

For screening the 24 additional non-B. ovatus strains, 3 mg of orange fiber was seeded into a deep 96-well plate; a liquid handling robot (Precision XS, Biotek) added 0.6 mL of Wilkins-Chalgren anaerobe broth to each well (yielding a final concentration of 5 mg orange fiber/mL). Each well was subsequently inoculated with 50 μL of a stationary phase culture of the bacterial strain targeted for screening and sealed with foil. The screen was performed in triplicate and carried out under identical conditions as the 14-strain experiment.

Genomic DNA extraction and purification. Bacterial isolates were inoculated into TYG media and were grown at 37° C. in an anaerobic chamber with an atmosphere of 75% N2, 20% CO2 and 5% H2 until reaching stationary phase. A 10 μL aliquot was transferred into 10 mL of fresh TYG media and was incubated for 72 hours under anaerobic conditions without shaking. A fraction of the broth was removed for full-length 16S sequencing to confirm the identity of culture isolates, and the remaining growth was spun down at 3,000 G for 5 minutes, yielding a 10-50 mg cell pellet, which was transferred to a 2 mL cryo-tube for DNA extraction. A 3.97 mm steel ball and 250 μL of 0.1 mm zirconia/silica beads were added to the tube along with a 500 μL mixture of 25:24:1 parts phenol:chloroform:isoamyl alcohol (pH 7.8-8.2), 210 μL of 20% SDS, and 500 μL of 2× buffer A (200 mM NaCl, 200 mM Trizma base, 20 mM EDTA). Samples were bead-beat for 1 minute in a Biospec Minibeadbeater-96 and were then centrifuged at 3220 g for 4 minutes. Following centrifugation, 420 μL of aqueous phase was transferred to a deep 96-well plate for subsequent DNA isolation. DNA was isolated using a QlAquick 96-well PCR purification kit with liquid handling performed using a Biomek FX robot. DNA was eluted from the column in 70 μL Tris-EDTA (TE) buffer and was quantified with a Quant-iT dsDNA broad range kit.

Long-read library preparation and sequencing. Approximately 1 μg of genomic DNA from each isolate was transferred into a 96-well, 0.8 mL, deep-well plate and was prepared for long-read sequencing using a SMRTbell Express Template Prep Kit 2.0 from Pacific Biosciences (PacBio) as described by the manufacture's guidelines for preparing HiFi Libraries from low DNA input, with adaptations for 96-well plate format. Purified DNA was of appropriate quality (DIN range: 6.8-7.9) and size (range of median peak size: 14.1-23.8 kb) for HiFi library preparation; therefore, no DNA shearing or size selection was performed prior to template preparation. All DNA handling and transfer steps were performed with ART wide-bore, genomic DNA pipette tips. Initial steps were performed as described in the PacBio protocol, including removal of single stranded overhands, DNA damage repair, end repair, and A-tailing. Barcoded adapters were ligated to A-tailed DNA fragments by overnight incubation at 20 C and were then treated with the SMRTbell Enzyme Cleanup Kit to remove damaged or partial SMRTbell templates. Ligated templates were purified, and size selected with 0.45× AMPure PB beads (45:100, AMPure beads:sample), and the size-selected libraries were pooled to yield equal genome coverage (3-6 libraries/pool). A second round of size selection with 0.45× AMPure PB beads was performed after pooling, and DNA was eluted in 12 μL of PacBio elution buffer.

Pooled libraries were quantified by Qubit, and the size distribution was evaluated on an Agilent TapeStation using Genomic DNA ScreenTape. The median fragment size for the 4 library pools ranged from 14.5 kb to 16.9 kb. Each library was sequenced on a Sequel System from Pacific Biosciences using a Sequel Binding Kit 3.0 and Sequencing Primer v4 with 24 hours of data collection.

Genome assembly and annotation. Samples were demultiplexed and Q20 circular consensus sequencing (CCS) reads were generated using a Cromwell workflow configured in SMRT Link. Genomes were assembled using Flye v2.8.1 with hifi-error set to 0.003, min-overlap set at 2000, and other options set to default. Genome quality was evaluated using checkm and annotated using the RASTtk pipeline.

Microbial RNA-Seq. Samples were prepared for microbial RNA-seq as described herein, except under the following conditions: a) Bacteroides ovatus TSDC 17.2 was grown in TYG, TYG without hemin, and MEGA media (b) Bacteroides ovatus 115, TYG was grown with or without 5 mg/ml orange fiber under quadruplicate conditions (n=4). A volume of 10 mL of 72-hour growth was centrifuged to yield 10-50 mg of pelleted bacteria, which was extracted by phenol chloroform as described herein.

A 3.97 mm steel ball and 250 μL of 0.1 mm zirconia/silica beads were added to each sample tube along with a 500 μL mixture of 25:24:1 parts phenol:chloroform:isoamyl alcohol (pH 7.8-8.2), 210 μL of 20% SDS, and 500 μL of 2× buffer A (200 mM NaCl, 200 mM Trizma base, 20 mM EDTA). Samples were then bead-beat for 1 minute in a Biospec Minibeadbeater-96 and were centrifuged at 3220 g for 4 minutes. A 100 μL fraction of the aqueous phase was transferred to a deep 96-well plate along with 70 μL isopropanol and 10 μL 3M NaOAc, pH 5.5 and was mixed by pipetting 10-times. The crude DNA/RNA mixture was chilled at −20° C. for approximately 1 hour and then centrifuging at 3220×g at 4° C. for 15 minutes before removing 210 μL of the supernatant to yield nucleotide-rich pellets. A Biomek FX robot was used to add 300 μL Qiagen Buffer RLT to the pellets and resuspend the RNA/DNA by pipetting up and down 50-times. A 400 μL volume was transferred to an AllPrep 96 DNA plate and was centrifuged at 3220 RCF for 1 min at room temperature. The RNA flow-through was purified as described in the AllPrep 96 protocol; DNA was then eluted from the column and retained.

Libraries were prepared from extracted RNA using the Illumina Stranded Total RNA Prep Ligation with Ribo-Zero Plus and were sequenced on an Illumina Next-Seq instrument using single end 75-nucleotide reads (1.33×10⁷±1.06×10⁶ reads/microbial sample). Reads were aligned to assembled genomes using bowtie. The resulting counts table was passed onto the R package DESEQ2 for differential gene expression analysis, where results were filtered as described. Sequence-based comparisons between genes expressed and/or present in B. ovatus strain TSDC 17.2 with B. ovatus strain 115, as well as the other B. ovatus strains were subsequently carried out on the SEED system, where a bidirectional BLAST search was carried out setting B. ovatus strain TSDC 17.2 as the reference genome for comparison. Annotation of PULs and regulon analysis were carried out as described.

Sample extraction for mass spectrometric analyses. All samples were maintained on liquid nitrogen throughout the extraction process. Frozen tissue was broken into small pieces and ground into a fine powder using a mortar and pestle. The powder was aliquoted into open-capped tubes (Reinforced, Thermo) pre-chilled in liquid nitrogen. Each sample was added to a 20 times weight volume of methanol along with 3-5 stainless steel beads (2.8 mm, Biospec) in a reinforced tube (Benchmark Scientific, catalog number D1031-RF) and placed on a pre-chilled block (20° C.). For gut contents, feces and in vitro screening samples, tubes were shaken using a Biospec bead beater for 4 minutes. For host tissues, tubes were shaken using a Biospec bead beater for two cycles of 4 minutes each, switching to a new chilled block each time that the bead beater was activated. For each plasma sample, a 40 μL aliquot was added to 4 mL of extraction solution (40% methanol in water) followed by addition of 20 μL of 100 nM tricarboxylic acid. After a 10-minute incubation at room temperature, samples were briefly vortexed and then centrifuged at 12,000×g for 10 minutes at 4° C.; 200 μL of the resulting supernatant was transferred to a 2 mL glass tube (Agilent) and dried in a speed vacuum at room temperature for two hours. The dry extract was reconstituted in 100 μL of 90% water/10% acetonitrile and stored at −4° C. prior to injection into a mass spectrometer.

Untargeted LC-Qtof-MS. Untargeted metabolomics was performed using an Agilent 1290 LC system coupled to an Agilent Model 6545 Qtof mass spectrometer (Santa Clara, CA). Five μL of each sample extract for positive ESI ionization was injected onto a BEH C18 column (2.1×150 mm, 1.7 μm, Waters Corp., Milford, MA) that was heated to 35° C. The mobile phase consisted of 0.1% formic in water (A) and 0.1% formic acid in acetonitrile (B). A flow rate of 0.3 mL/minute was applied (gradient program: from 0 to 14 minutes, mobile phase B eluted from 5% to 100%, followed by 3 minutes at 100% of B). An equilibration time of 3 minutes was used. Data were collected in positive ESI ionization modes in the range from m/z 50 to 1000, and m/z 150 to 650 for MS full-scan analysis and MS/MS analysis, respectively. The key parameters of Qtof were set as the following: nozzle voltage, 1000 V; capillary voltage, 3000 V; drying gas, N2; drying gas flow rate, 10.0 L/min; collision gas, high purity N2; drying gas (N2) temperature, 325° C.; vaporizer/sheath gas temperature, 350° C.; sheath gas flow rate, 12 L/min. To ensure accurate mass measurements, reference masses m/z 121.0509 and 922.0098 were automatically delivered using a dual ESI source during analyses. The mass accuracy of the LC-MS system used herein was generally better than 4 ppm. Samples were randomly analyzed.

The resulting raw data sets were deconvoluted using MassHunter Profinder B.08.00 software (Agilent Technologies, Santa Clara, CA) which generated a list of molecular features. These features were subsequently filtered using in-house scripts in order to identify those that were only present in all samples obtained from mice that were colonized and fed the orange fiber supplemented HiSF-LoFV diet. Initial characterization of the resulting subset of features was performed by monoisotopic mass search in METLIN (metlin.scripps.edu) and HMDB (hmdb.ca). These features were fragmented by targeted MS/MS with collision energy from 0 to 40 V. Final metabolite identification was performed by co-characterization with standards.

Targeted LC-QqQ-MS. N-methylserotonin—Five microliters of sample extract were injected into a 1290 Infinity II UHPLC system coupled to a Model 6470 Triple Quadrupole LC/MS system equipped with a Jet Stream electrospray ionization source (Agilent Technologies). Chromatographic separation was performed on a ZORBAX Extend-C18, 2.1×50 mm, 1.8 μm column (Agilent Technologies) and the following gradient conditions: 5-95% solvent B (methanol/0.1% formic acid); 0-3 minutes at a flow rate of 0.2 mL/minute. Mass spectra were acquired in positive mode and quantification transitions for N-methylserotonin at 191→160.

Other metabolites—Tissue (at least 10 mg) was placed in a reinforced 2 mL tube. A 20 times weight volume of extraction solvent was added (40% acetonitrile, 40% methanol, 20% water) and the tissue was disrupted as described herein. Samples were centrifuged at 12,000×g for 10 minutes at 4° C. A 200 μL aliquot of the resulting supernatant was transferred to a 2 mL glass tube and dried in a speed vacuum at room temperature (25° C.) for two hours. The dry extract was reconstituted in 100 μL of 90% water/10% acetonitrile and stored at −4° C. prior to injection; 5 μL was injected into a 1290 Infmity II UHPLC system coupled to a 6470 Triple Quadrupole LC/MS system equipped with a Jet Stream electrospray ionization source (Agilent Technologies). Chromatographic separation was performed on an Agilent ZORBAX Extend C18, 2.1×150 m, 1.8 μm column, using the following gradient conditions: mobile phase A, 10 mM tributylamine and 15 mM acetic acid in 3% methanol (v/v); mobile phase B, 10 mM tributylamine and 15 mM acetic acid in 100% methanol; 0% solvent B (0-2 minutes); 0-20% solvent B (2-7.5 minutes); 20-45% solvent B (7.5-13 minutes); 45-99% solvent B (13-20 minutes); 99-0% solvent B (20-22 minutes) at a flow rate of 0.25 mL/minute. Mass spectra were acquired in negative mode using the following conditions: capillary voltage set at 2000V; nitrogen as the nebulizer gas (45 psi); drying gas flow rate and temperature of 13 L/minute and 250° C., respectively; sheath gas flow rate and temperature of 12 L/minute and 325° C. Transitions were taken from the Agilent Metabolomics dMRM Database.

Quantification and statistical analysis. Details regarding statistical tests used, replicates and representation of means and standard deviations are provided herein throughout in the specification text, figure legends, and tables.

Data and code availability. Annotated B. ovatus genomes, microbial RNA-seq, and COPRO-Seq, liver and colonic RNA-Seq datasets from gnotobiotic mice have been deposited at the European Nucleotide Archive (ENA; ebi.ac.uk/ena) under accession number PRJEB40461. Metabolomics data are available in the EMBL-EBI MetaboLights database (identifier MTBLS2331). Shotgun and 16S rDNA amplicon sequencing datasets generated from human fecal DNAs are available in ENA (study accession PRJEB44020).

Enzymatic screening of citrus & plant samples. All enzymatic assays are carried out using a total volume of 10 ml in water, 50 mg of plant fiber material tested and 700 active units of the same cellulase enzyme mixture (Sigma) as described herein. Samples were processed based on their condition, divided as follows. Citrus fibers sourced from various citrus types, described in Table S9 as “Ground,” are added directly to the enzymatic assay, while citrus fibers described as either “Granule” or “Whole” are first manually pulverized into a fine powder via mortar and pestle. The commercially available fiber source is classified as “Ground” if it was already processed into fine powder, or otherwise small enough granules in which individual pieces cannot be manually picked up via forceps. The commercially available fiber source is classified as “Granule” or “Whole” if individual fragments or pieces of the fiber is readily discernable, where “Granule” are applied to visibly uniform samples and “Whole” applied to all other samples. Assays were performed in triplicate for each sample type tested after an incubation of 72 hours. The extraction and subsequent detection of N-methylserotonin is described herein elsewhere.

Enzymatic assays were carried out in identical fashion as Table S9, differing only in the preparation of the materials tested (see Table S10). For the locally sourced citrus experiments, the peel, pulp, and fruit of the various citruses were separated into their respective portions, then chopped and ground to a fine powder/paste where then 250 mg of this raw material are applied to the enzymatic digestion assay with or without enzyme (as noted in Table S10 where appropriate). Samples designated “Skin” refer to solely the epicarp/flavedo, inedible, hardened portion of the peel that are visually distinctive by color. Samples designated “Pulp” refer to the mesocarp/albedo, the inner layer of the skin, as well as endocarp membranes which overlaps with the edible portion of the fruit. Samples designated “Fruit (with pulp)” refer to the edible portion of the citrus fruit, or segments of the endocarp containing the juice vesicles, which also includes the membranous endocarp layer. For liquid samples (designated “Juice”), 50 μL juice, squeezed from the locally sourced citrus fruits or else taken directly from the commercially available source are either first passed through a 0.22 μm filter syringe or else directly applied to the enzymatic digestion assay process via identical methods described in Table S9.

Enzymatic assays were carried out in identical fashion as Table S9, differing only in the preparation of the materials tested (see Table S11(A-E)). In case of dried materials such as grains or various herbs, the samples are ground to a fine powder via mortar and pestle. In case of wet materials such as various raw fruits and vegetables, the samples are instead chopped to a fine paste-like consistency where no visual distinction could be made of individual components. 1 g of each processed material (dried weight or wet weight) is then applied to the enzymatic digestion assay as described in Table S9. Sample types marked “Fiber” or “Pre-Ground” indicate that the samples come from a commercially prepared source, where some degree of mechanical or culinary application has been carried out. 50 mg of this type of material is applied to the enzymatic assay as described in Table S9 with no additional processing prior to extraction.

### Star Methods Key Resource Table

TABLE 1A
Strains
STRAINS

Agrobacterium radiobacter DSM 30147
Alicyclobacillus acidiphilus DSM 14558
Anaerococcus vaginalis TSDC20.1-1.1
Anaerofustis stercorihominis TSDC20.1-1.1
Bacteroides caccae TSDC 17.2-1.2
Bacteroides caccae TSDC20.2-1.1
Bacteroidesfinegoldii TSDC 17.2-1.1
Bacteroides fragilis TSDC20.1-1.1
Bacteroides intestinalis TSDC 17.2-1.1
Bacteroides intestinalis TSDC20.1-1.1
Bacteroides massiliensis TSDC 17.2-1.1
Bacteroides ovatus 115
Bacteroides ovatus TSDC 17.2-1.1
Bacteroides ovatus VPI-435
Bacteroides ovatus VPI-B4-11
Bacteroides ovatus VPI-C1-45
Bacteroides ovatus VPI-C16-22
Bacteroides ovatus VPI-C2-26
Bacteroides ovatus WH208
Bacteroides ovatus WH214
Bacteroides ovatus WH514
Bacteroides ovatus WH604
Bacteroides ovatus WH711
Bacteroides thetaiotaomicron TSDC 17.2-2.2
Bacteroides thetaiotaomicron TSDC20.2-1.1
Bacteroides uniformis TSDC20.1-1.1
Bacteroides uniformis TSDC20.2-1.1
Bacteroides vulgates TSDC 17.2-1.1
Bifidobacterium longum TSDC20.1-1.1
Bifidobacterium longum TSDC20.2-1.1
Clostridiales TSDC20.1-1.1
Clostridium bolteae TSDC20.2-1.1
Clostridium hylemonde TSDC20.2-1.1
Clostridium scindens TSDC20.1-1.1
Collinsella aerofaciens TSDC 17.2-1.1
Dialister invisus TSDC20.1-1.1
Dorea longicatena TSDC20.1-1.1
Eggerthella lenta TSDC20.1-1.1
Escherichia coli TSDC 17.2-1.2
Escherichia coli TSDC20.1-1.1
Finegoldia magna TSDC20.1-1.1
Odoribacter splanchnicus TSDC 17.2-1.2
Parabacteroides distasonis TSDC 17.2-1.1
Ruminococcaceae sp. TSDC 17.2-1.2
Ruminococcus albus TSDC 17.2-1.4
Ruminococcus gnavus TSDC20.2-1.1
Subdoligranulum variabile TSDC 17.2-1.1
Subdoligranulum variabile TSDC20.2-1.1
Veillonella parvula TSDC20.2-1.1
Veillonella TSDC20.1-1.1
Veillonella TSDC20.2-1.1

TABLE 1B
Reagents and Resources

| | |
|---|---|
| REAGENT or RESOURCE | SOURCE/IDENTIFIER |

Chemicals, Peptides, and Recombinant Proteins

| | |
|---|---|
| N-methylserotonin | Santa Cruz Biotech, Cat# sc- |
| Tryptophan | Sigma, Cat# 93659 |
| Tryptamine | Sigma, Cat# 193747 |
| Serotonin | Sigma, Cat# 14927 |
| Dimethylserotonin | Sigma, Cat# B-022 |
| Trimethylserotonin | Sigma, Cat# H-133 |
| Methyltryptamine | Santa Cruz Biotech, Cat# sc- |
| S-adenosyl methionine | Santa Cruz Biotech, Cat# sc- |
| Cellulase | Sigma, Cat# C2730 |
| Hemicellulose | Megazyme, Cat# E-GERF |
| Xylanase | Megazyme, Cat# E-XYAN4 |
| exo-Inulinase | Megazyme, Cat# E-EXOIAN |
| endo-Inulinase | Megazyme, Cat# E-ENDOIAN |
| beta-Xylanase | Megazyme, Cat# E-XYNBS |
| endo-Polygalacturonananase (M2) | Megazyme, Cat# E-PGALUSP |
| alpha-Amylase | Megazyme, Cat# E-ANAAM |
| Lichenase | Megazyme, Cat# E-LICHN |
| Alginate Lyase | Megazyme, Cat# E-ALGLS |
| beta-Mannanase | Megazyme, Cat# E-BMANN |
| endo 1,4 beta D-galactanase | Megazyme, Cat# E-GALCJ |
| endo 1,5 alpha L-arabinanase | Megazyme, Cat# E-EARAB |

Critical Commercial Assays

| | |
|---|---|
| Glycogen Assay kit | Sigma, Cat# MAK016 |
| Takara Nucleospin RNA Plus kit | Takara, Cat# 740984 |
| Agilent RNA 6000 Pico kit | Agilent, Cat# 5067-1513 |
| Illumina TruSeq Stranded Total RNA | Illumina, Cat# 20040529 |
| SMRTBell Express Template Prep Kit 2.0 | Pacific Biosciences, Cat# 101-685- |
| SMRTBell Enzyme Clean up Kit | Pacific Biosciences, Cat# 101-746- |
| Ampure PB beads | Pacific Biosciences, Cat# 100-265- |
| Barcoded Overhang Adapter Kit- 8A, 8B | Pacific Biosciences, Cat# 101-628- |
| Q1Aquick 96 PCR Purification column (cat# | Qiagen, Cat# 28181 |

Deposited Data

| | |
|---|---|
| COPRO-Seq shotgun sequences | Present disclosure, PRJEB40461 |
| Tissue RNA-seq sequences | Present disclosure, PRJEB40461 |
| Microbial RNA-seq sequences | Present disclosure, PRJEB40461 |

Experimental Models: Organisms/Strains

| | |
|---|---|
| C57BL/6J mice (re-derived germ-free) | The Jackson |
| | Laboratory, Cat# 000664 |

Oligonucleotides

| | |
|---|---|
| COPRO-Seq PCR (forward) (SEQ ID NO: 1) | AAT GAT ACG GCG ACC ACC |
| | GAG ATC TAC ACT CTT TCC CTA |
| | CAC GAC GCT CTT CCG ATC T |
| COPRO-Seq PCR (reverse) (SEQ ID NO: 2) | CAA GCA GAA GAC GGC ATA |
| | CGA GAT CGG TCT CGG CAT TCC |
| | TGC TGA ACC GCT CTT CCG ATC T |

Software and Algorithms

| | |
|---|---|
| Agilent software | Agilent |
| QIIME v1.9.2 | qiime.org |
| COPRO-Seq pipeline | github.com/nmcnulty/COPRO-Seq |
| R | The R foundation, r-project.org/ |
| Prism v9.2 | Graphpad, graphpad.com |
| STAR | github.com/alexdobin/STAR |
| FeatureCounts | subread.sourceforge.net/ |
| Deseq2 | bioconductor.org/packages/release/ |
| | bioc/html/DESeq2.html |
| ClusterProfiler | bioconductor.org/packages/release/ |
| | bioc/html/clusterProfilr.html |
| Prokka | github.com/tseemann/prokka |
| RAST | rast.nmpdr.org/ |
| SMRT Link v9.0 | pacb.co m |
| Cromwell | cromwell.readthedocs.io/en/develop/ |
| Flye v2.8.1 | github.com/fenderglass/Flye |
| RASTtk | rast.nmpdr.org/rast.cgi |

Other

| | |
|---|---|
| Pea fiber | Rettenmaier, Cat# Pea Fiber EF |
| Orange fiber | Fiber Star, Cat# CitriFri 100 |
| Carmine red dye | Sigma-Aldrich, Cat# C1022 |
| Lysing Matrix F | MP Bio, Cat# 116915050-CF |
| Reinforced Tubes | Thermo-Fischer, Cat# NC0444131 |
| 96-well, round bottom, deep well palate | Axygen, Cat# P-DW-11-HC |
| ART 200G, Filtered, Sterile pipette tips | Thermo Scientific, Cat# ART |
| Genomic DNA ScreenTape | Agilent, Cat# 5067-5365 |
| Genomic DNA Reagents | Agilent, Cat# 5067-5366 |
| 4200 TapeStation | Agilent, Part# G2991BA |
| Quant-iT dsDNA broad range kit | Invitrogen, Cat# Q33130 |
| Sequel System | Pacific Biosciences, N/A |
| Procedure - SMRTBell Express Template Prep | Pacific Biosciences, Part# 101- |

### Results

A gnotobiotic mouse model reveals human gut bacterial liberation of N-methylserotonin from orange fiber. As a starting point for characterizing liberation of fiber-associated bioactive constituents by gut bacterial taxa, a commercial, food-grade source of orange fiber was selected (see Methods), derived from the byproducts of the juicing process; these byproducts include pulp cells, juice vesicles, segment membranes, rag/core and peel that are mechanically processed (washed with water, heated, dewatered, sheared) prior to drying. Importantly for the purpose of experiments for the present disclosure, the preparation had not been subject to chemical treatment or extraction; therefore, any proteins, lipids, and small molecules that are not removed by washing with water are retained in the preparation (see Table S1A,B for composition and glycosidic linkage analysis of constituent polysaccharides).

Two groups of adult C57BL/6J germ-free mice were colonized with a 14-member consortium of sequenced human gut bacterial strains and monotonously fed the HiSF-LoFV diet ad libitum, with or without supplementation with 10% (w/w) orange fiber, for 21 days. Two other groups of mice were maintained as germ-free; mice in one of these groups were fed the unsupplemented HiSF-LoFV diet while those in the other group consumed the 10% orange fiber-supplemented diet (n=5 animals/treatment group).

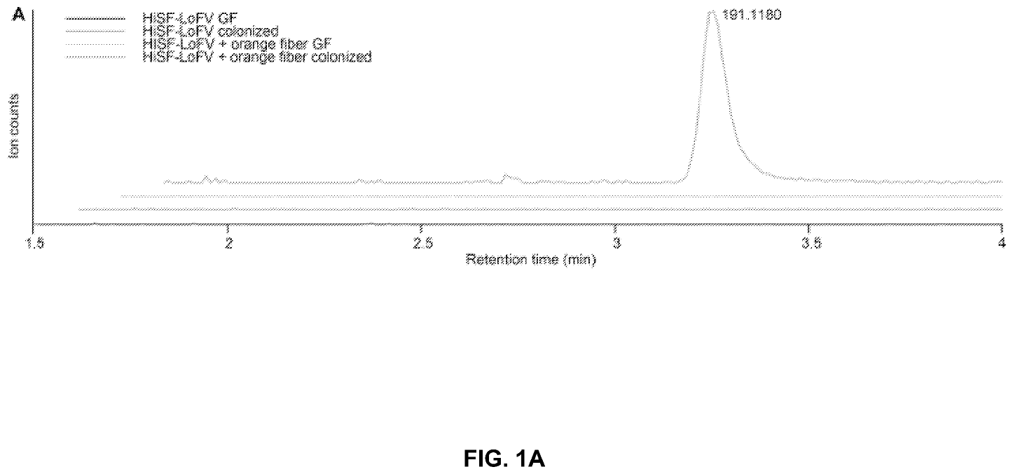

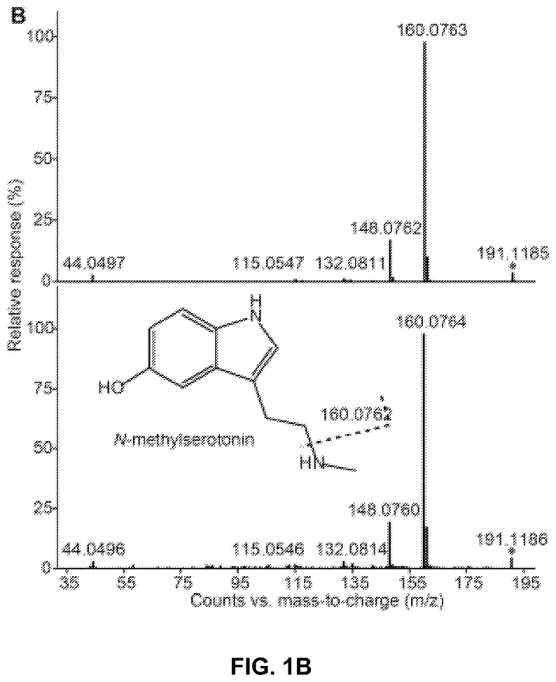

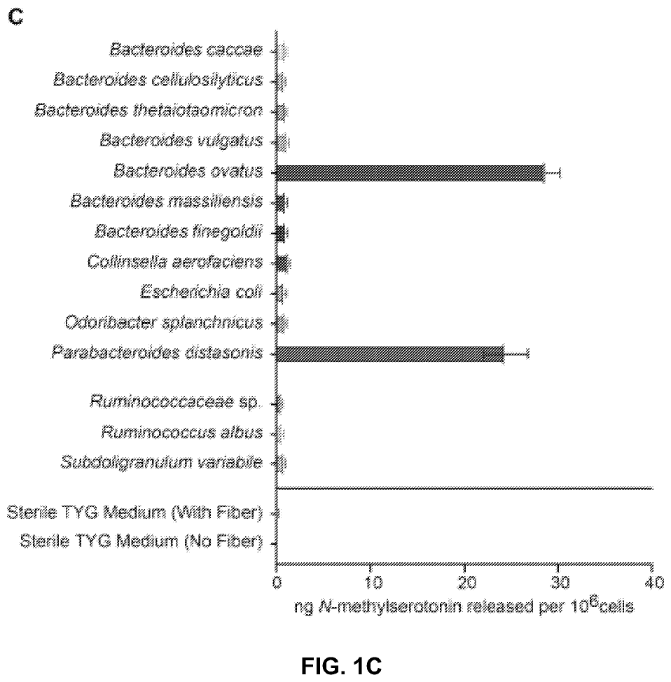

Untargeted liquid chromatography-quadrupole time-of-flight mass spectrometry (LC-Qtof-MS) of cecal contents harvested at the time of euthanasia revealed 116 features (m/z) that were increased at least 3-fold in colonized mice consuming the orange fiber-supplemented diet compared to the other three experimental groups (Table S2). A prominent feature with an m/z of 191.1186 was present at high abundance only in colonized mice fed the orange fiber-supplemented diet (FIG. 1A); it was tentatively identified as methylserotonin, with its major fragment (m/z 160.0760) consistent with methylation of its alkyl amine. Subsequent LC-Qtof/MS/MS co-characterization with a known standard confirmed that this compound was N-methylserotonin (FIG. 1B). N-methylserotonin has been previously identified in several plants, including black cohosh, Japanese pepper, and citrus fruits. There is very limited information on whether this compound has beneficial or potentially detrimental physiologic effects.

The experiment was repeated and further compared different groups of mice colonized with the 14-member consortium and fed the HiSF-LoFV diet supplemented with 10% orange fiber or with 10% pea fiber. The latter was a natural food-grade commercial preparation consisting of insoluble and soluble fibers as well as resistant starch (see Methods and Table S1A for composition). Targeted liquid chromatography-triple quadrupole mass spectrometry (LC-QqQ-MS) revealed that N-methylserotonin was present in significantly higher amounts in both the cecal and colonic contents and tissues of mice consuming the orange fiber-supplemented diet [173±14 ng/g and 130±13 ng/g (mean±SD) in cecal contents and cecal tissue, respectively and 139±13 ng/g and 164±25 ng/g in colonic contents and tissue, respectively] compared to their small intestine, liver, gastrocnemius muscle and kidney (<1 ng/g) or plasma (1.22±0.76 ng/mL). Non-targeted LC-Qtof-MS and targeted LC-QqQ-MS analysis revealed no statistically significant differences (p>0.05; t-test) in the levels of serotonin, dimethylserotonin, trimethylserotonin, 5-hydroxyindoleacetic acid, tryptamine, N-methyltryptamine, N,N-dimethyltryptamine, tryptophan, bufotenin or melatonin in small intestinal, cecal and colonic tissue or liver obtained from animals consuming the unsupplemented versus orange fiber-supplemented HiSF-LoFV diet, indicating that the host is unable to metabolize N-methylserotonin to these products. N-methylserotonin was below the limits of detection (<0.5 ng/g) in cecal or colonic contents or any of these intestinal and extra-intestinal tissues harvested from mice consuming the pea-fiber supplemented diet.

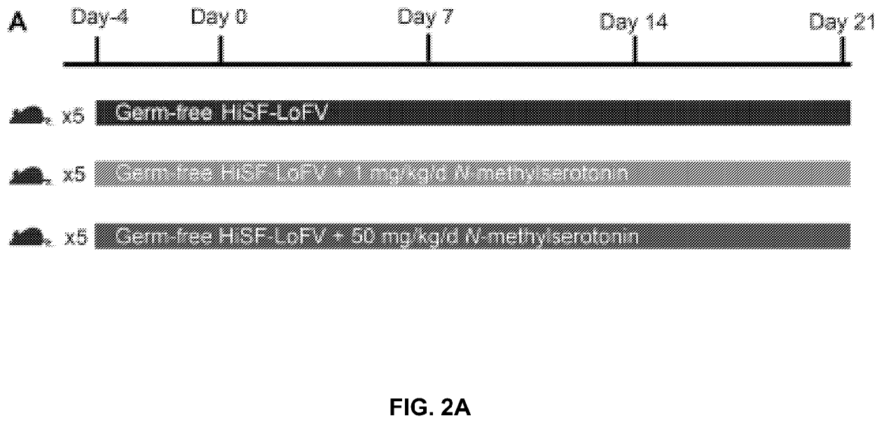

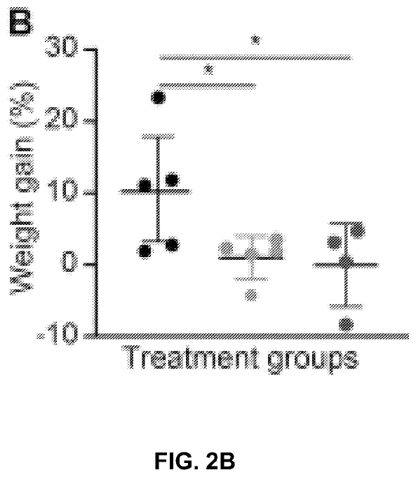

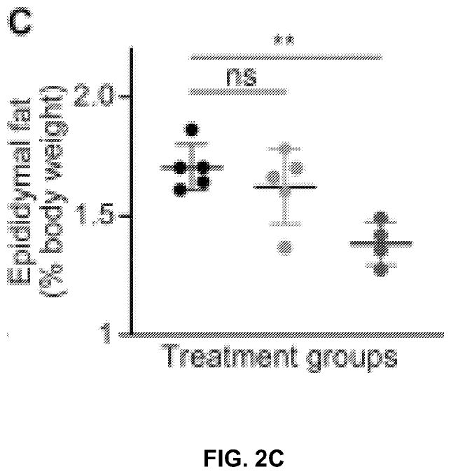

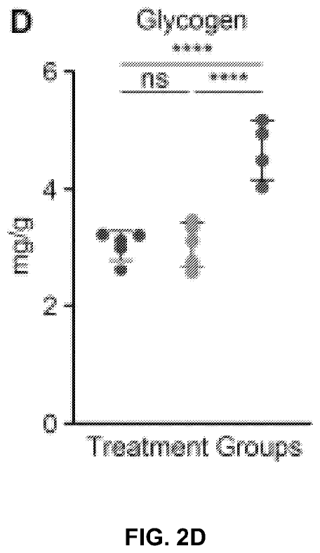

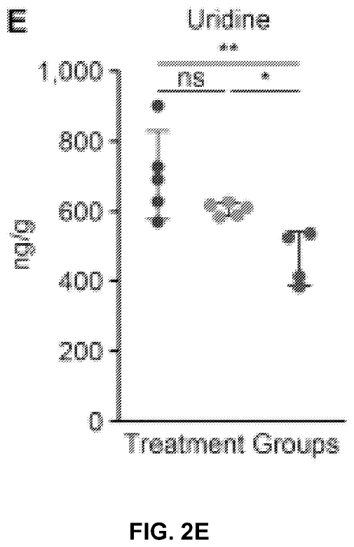

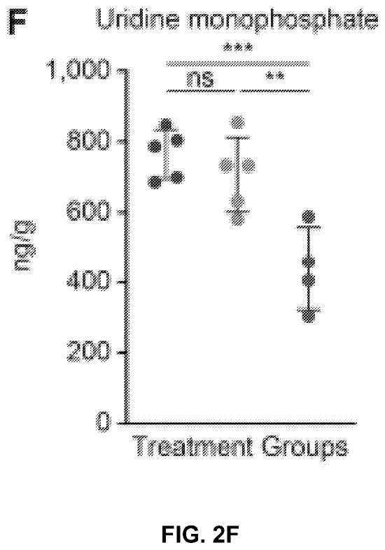

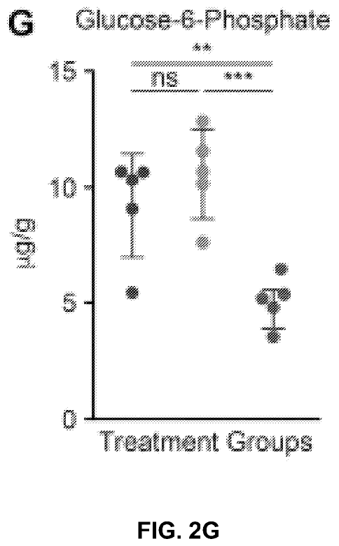

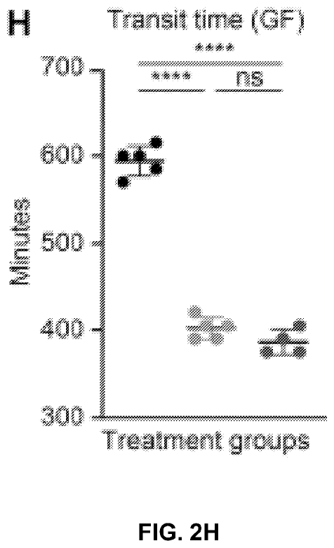

Host effects of N-methylserotonin. To characterize the effects of N-methylserotonin on host physiology and metabolism, 12-week-old germ-free C57BL/6J mice were fed the unsupplemented HiSF-LoFV diet and administered N-methylserotonin via their drinking water at doses of 1 mg/kg/day or 50 mg/kg/day for 21 days (FIG. 2A). The 1 mg/kg/day dose was experimentally determined to result in fecal N-methylserotonin levels that were equivalent to those documented in mice colonized with the 14-member community consuming the orange fiber-supplemented HiSF-LoFV diet (133.5±15 ng/g versus 131±19 ng/g feces, respectively; p=0.92, unpaired t-test). The 50 mg/kg/d dose was equivalent to the estimated total amount of N-methylserotonin consumed each day in the 10% orange fiber-containing diet (based on the yield obtained after in vitro enzymatic digestion of the fiber with T. reesei cellulase). A control group of germ-free animals did not receive any N-methylserotonin. Food and water intake were measured daily and remained consistent throughout the experiment among all three groups of mice.

While a statistically significant decrease in weight gain was observed with N-methylserotonin treatment (FIG. 2B), interpreting this result is confounded by the abnormally large contribution of the cecum to body weight in germ-free mice. However, compared to untreated controls, oral administration of N-methylserotonin resulted in a statistically significant reduction in epididymal fat mass at the higher dose but not the lower dose (FIG. 2C).

The higher dose also produced statistically significant increases in liver glycogen (FIG. 2D) and statistically significant decreases in its metabolic precursors, uridine and uridine monophosphate (FIG. 2E,F), which the lower dose did not. Administration of the higher dose resulted a statistically significant decrease in liver glucose-6-phosphate (FIG. 2G), a key metabolic intermediate formed from either glycogenolysis or gluconeogenesis that is known to directly impact levels of glycogen in the liver, while the lower dose did not. Based on these results, untreated control animals and those that received 50 mg/kg/d of N-methylserotonin were used to perform RNA-Seq on liver and colon.

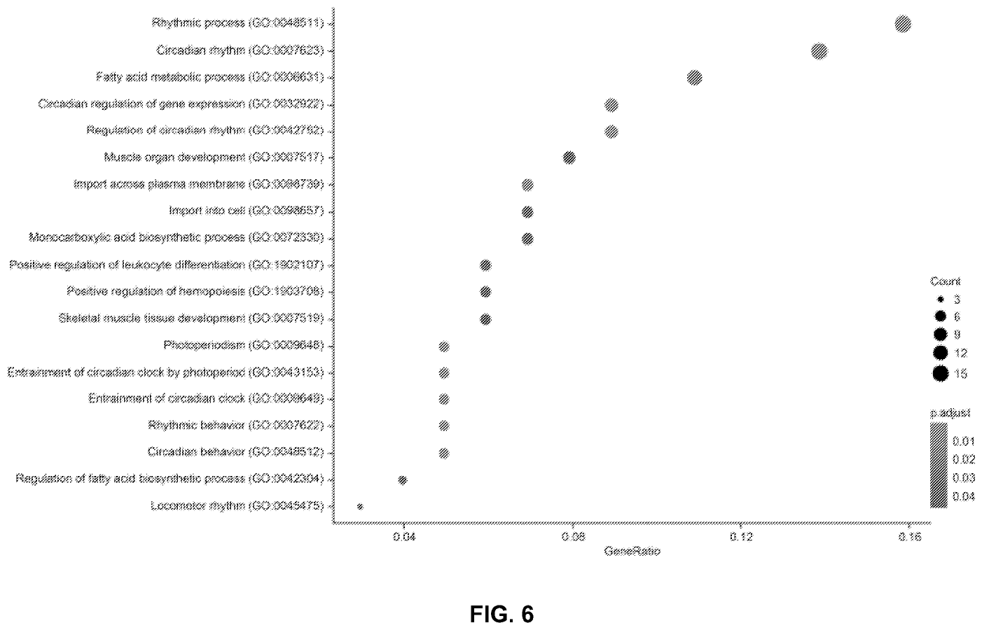

A total of 716 genes exhibited statistically significant differences in their expression in the livers of N-methylserotonin-treated compared to untreated mice (FDR adjusted p-value<0.05; see Methods and Table S3A,B). Gene-set enrichment analysis and over-representation analysis (Methods) of all statistically significant differentially expressed genes (FDR adjusted p-value<0.05) revealed that GO Biological Pathway terms pertaining to circadian rhythm and fatty acid metabolism were the most significantly enriched (FIG. 6). Effects of N-methylserotonin on circadian rhythm-related genes included significantly decreased expression of Arntl and Clock [see Table S3 for loge-fold change and FDR adjusted p-values], both of which have been linked to suppressed gluconeogenesis and lipogenesis. Consistent with this observation, there were significant increases in expression of their regulators Per2, Per3, and Nr1d2. Per2 promotes glycogen synthesis. Moreover, genetically engineered disruption of Per3 is associated with resistance to leptin with resulting weight gain, while its deletion directly leads to increased adipogenesis. Nr1d2 acts a repressor of Nfil3; its statistically significant increased expression with N-methylserotonin treatment is associated with statistically significantly decreased hepatic levels of Nfil3 mRNA. Nfil3 serves as an important link between the gut microbiota, intestinal epithelial lipid metabolism and body composition. Nfil3 expression exhibits microbiota-modulated diurnal oscillation in epithelial cells via group 3 innate lymphoid cells, Stat3 and epithelial clock components, with accompanying changes in epithelial lipid absorption and export. Moreover, genetic ablation of Nfil3 attenuates high fat diet-induced obesity in mice.

Seven hundred and forty-eight genes exhibited statistically significant differences in their expression in the colonic tissue of N-methylserotonin-treated versus untreated mice (FDR-adjusted p-value<0.05; Table S3C,D); they include Nr1d2, Per3, Per2, Arntl and Clock. In vitro studies have indicated that N-methylserotonin binds to various serotonin (5-hydroxytryptamine) G protein-coupled receptors, including 5-Htr7 and 5-Htr2A. RNA-Seq analysis of colon did not reveal any statistically significant effects of N-methylserotonin administration on colonic expression of its known (Htr7, Htr2A) or related (Htr3, Htr4, Htr5 and Htr6) receptors.

Glutamate levels were significantly higher in colonic tissue harvested from germ-free mice receiving 50 mg/kg/day of N-methylserotonin compared to untreated controls (21±1.4 versus 11±0.9 ng/mg tissue, respectively; p<0.01, unpaired t-test). Glutamate is known to degrade Arntl when directly applied to tissue slices. Knockout of the Per3 homolog Per2 reduces expression of the glutamate transporter (Eaatl, Slc1A3) and uptake of glutamate in the brain. These observations raise the possibility that one way that N-methylserotonin might influence colonic circadian regulators is through its effects on glutamate levels in this tissue.

Circadian rhythm-related genes are known to be expressed in the myenteric plexus which coordinates colonic motility. Orally administered carmine red was used to determine the gastrointestinal transit time on day 17 of the 21-day experiment in germ-free mice whose drinking water was supplemented with 1 mg/kg/day or with 50 mg/kg/day of N-methylserotonin. The assay revealed that both doses of N-methylserotonin produced equivalent reductions in transit time (i.e., increased motility) compared to the untreated control group (p<0.0001, one-way ANOVA; FIG. 2H).

Bacterial strains capable of mining of N-methylserotonin from orange fiber in vitro. Given that detection of N-methylserotonin was dependent on colonization with the bacterial consortium and consumption of orange fiber, it was next investigated which community members were responsible for its appearance. Each of the 14 community members was grown in monoculture to stationary phase in TYG medium; 10⁵ cells of each organism were incubated in 10 mL of a 5 mg/mL suspension of orange fiber for 8, 24, 48, 72 and 168 hours. N-methylserotonin was quantified using targeted LC-QqQ-MS. N-methylserotonin rose from levels that were not significantly above background at the 8 h time point (background determined by measurements of control incubations containing sterile TYG medium), to levels that reached a maximum at the 72-hour time point. At this time point, Bacteroides ovatus strain TSDC 17.2 and a strain of Parabacteroides distasonis yielded similar quantities of product (29±1 and 24±2 ng N-methylserotonin/10⁶ cells, respectively). In contrast, the 12 other strains yielded<1.5 ng/10⁶ cells—an amount that was not appreciably higher than background levels (triplicate incubations/organism; FIG. 1C). Cultures grown in Wilkins-Chalgren anaerobe broth yielded results that were similar to those obtained with TYG medium (Table S4A), while testing each of these 14 organisms in an another nutrient rich medium (Mega medium 2.0) resulted in N-methylserotonin levels ranging from 5-25 ng/10⁶ cells for P. distasonis, Bacteroides finegoldii and Collinsella aerofaciens (Table S4A). Given its capacity to support the growth of a number of cultured anaerobic gut bacterial taxa, 24 other phylogenetically diverse human gut bacterial strains were screened in Wilkins-Chalgren anaerobe broth containing 5 mg/mL of orange fiber; none yielded amounts of N-methylserotonin significantly above background (<0.5 ng/10⁶ cells) (Table S4B).

Several other experiments were performed to characterize in vitro N-methylserotonin liberation by members of the 14 strain consortia. Adding either (i) 10⁸ heat-killed cells of either B. ovatus TSDC 17.2 or the P. distasonis strain that had been grown to stationary-phase in TYG, or (ii) lysates prepared by bead-beating of 10⁸ cells harvested from monocultures of each organism in TYG, or (iii) conditioned medium harvested from stationary phase TYG monocultures of each organism, to fresh TYG with orange fiber for 72 hours failed to yield levels of N-methylserotonin above background (Table S4C). These experiments indicate that mining requires intact viable cells. When N-methylserotonin was added at a concentration of 5 ng/ml to monocultures of the 14 strains that had been grown to stationary phase in TYG medium without orange fiber, no appreciable degradation was observed over a 72-hour period (3 replicate assays/organism/experiment; 97±2% of input N-methylserotonin remaining intact/unmodified; triplicate incubations/condition; Table S4D). Evidence that B. ovatus TSDC 17.2 was not capable of synthesizing N-methylserotonin was also obtained. A homology-based search of the bacterial genome failed to reveal gene candidates involved in serotonin biosynthesis and metabolism. Moreover, when the organism was cultured in TYG medium supplemented with either tryptophan, tryptamine, serotonin, dimethylserotonin, trimethylserotonin, methyltryptamine, or S-adenosyl methionine (see Methods), N-methylserotonin was not detected above background after either 24 or 72 hours. Incubating this strain in TYG medium supplemented with either pea fiber, or two other commercial dietary fiber preparations (apple pectin or oat beta glucan) also did not yield levels of N-methylserotonin above background.

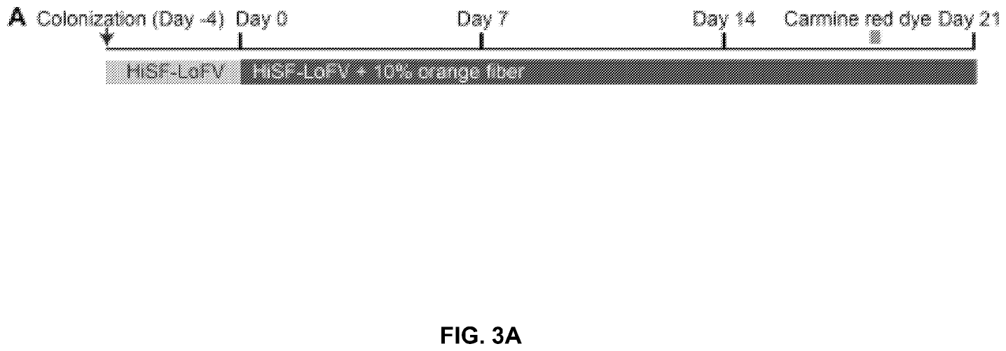

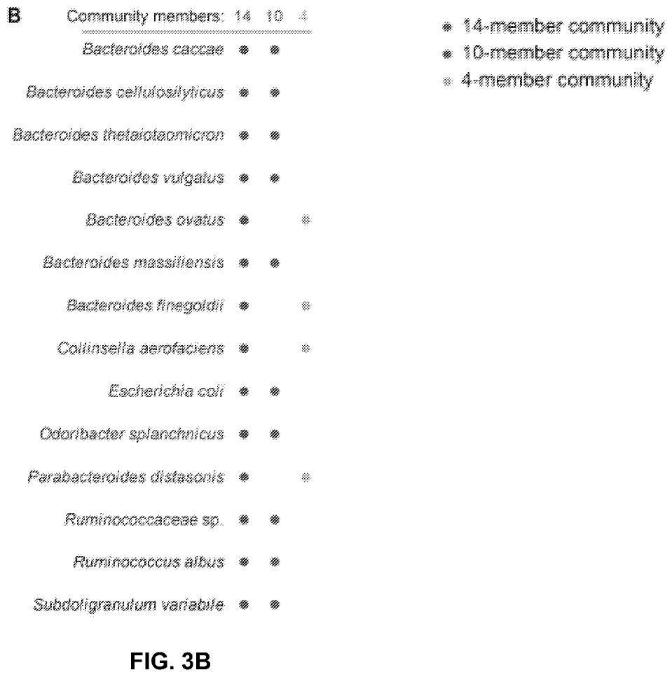

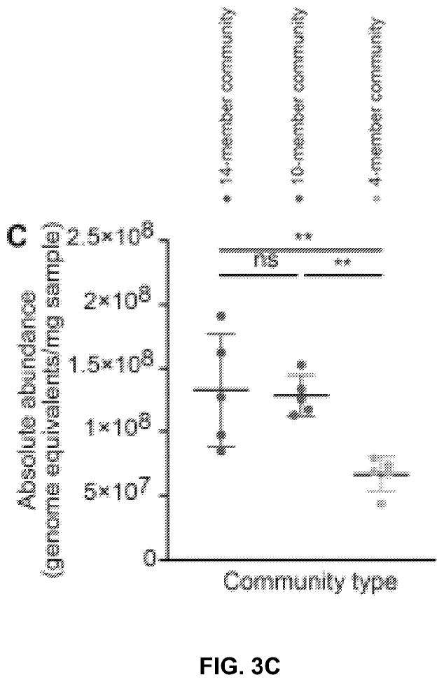

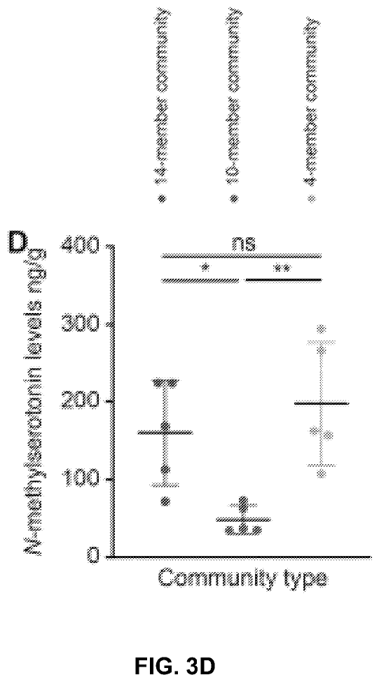

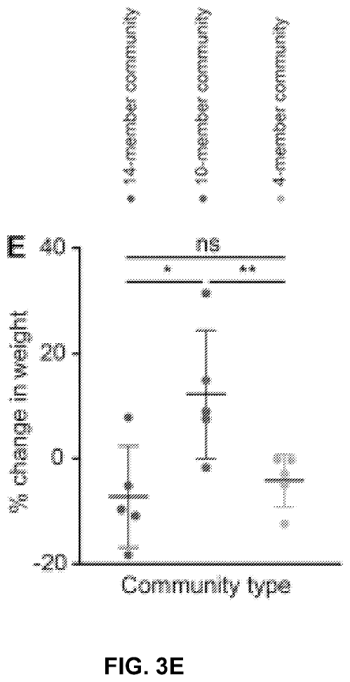

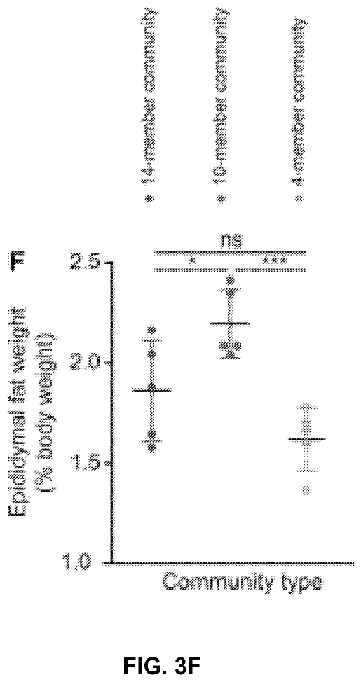

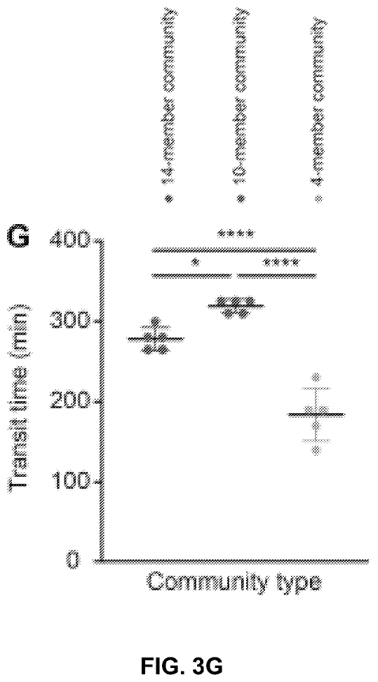

Based on these in vitro findings, adult C57BL/6J germ-free mice were colonized with (i) a 4-member consortium comprised of the B. ovatus, P. distasonis, B. finegoldii and C. aerofaciens strains with in vitro mining activity, or (ii) the full 14-member consortium, or (iii) the 10 remaining strains from the 14-member consortium. Three days after gavage, animals (n=5/group) were switched from the unsupplemented HiSF-LoFV diet to a HiSF-LoFV diet supplemented with 10% (w/w) orange fiber. This diet was then administered ad libitum for 21 days (FIG. 3A,B). Short-read shotgun sequencing of DNA isolated from fecal samples collected at the time of euthanasia revealed that all strains in each consortium were able to colonize recipient animals (see Table S5 for their absolute abundances). The total biomass (bacterial genome equivalents/g feces) in mice harboring the 4-member consortium was 2-fold lower than in animals colonized with the 14-member consortium (p<0.01; one-way ANOVA) while there was no significant difference in bacterial load between animals hosting the 10- and 14-member communities (p=0.81, FIG. 3C). Targeted LC-QqQ-MS analysis of fecal samples obtained at the time of euthanasia revealed that levels of N-methylserotonin in mice colonized with the 4-strain consortium were equivalent to those in animals harboring the full 14-member community and significantly higher than in mice colonized with the 10-member consortium (p=0.002; one-way ANOVA; FIG. 3D). Animals colonized with the 4-member consortium had a significantly lower epididymal fat pad mass compared to mice colonized with the 10-member consortium (p=0.007 and p=0.001, respectively; one-way ANOVA). No significant differences in adiposity were noted between mice harboring the 4- and 14-member communities (FIG. 3E,F). Mice colonized with the 4-member consortium also had a statistically significant reduction in gut transit time compared to animals containing the 10-member community [184±32 minutes (mean±SD) versus 319±8 minutes, respectively; p<0.0001, one-way ANOVA). The 14-member community was associated with transit times (278±14 minutes), that were also significantly shorter compared to mice with the 10-member community (p=0.025) yet were still significantly longer compared to mice harboring the 4-member community (p<0.0001) (FIG. 3G).

Identifying genes involved in release of N-methylserotonin from orange fiber. Reasoning that microbial disruption of complex polysaccharides in fibers might be needed to liberate sequestered N-methylserotonin, a set of experiments was performed where 50 mg of orange fiber was incubated separately with 13 commercially available glycoside hydrolase preparations (see Methods). The greatest amount of N-methylserotonin was recovered when orange fiber was incubated with a preparation containing endoglucanases and cellulases from Trichoderma reesei (total yield; 2728±26 ng/50 mg orange fiber) (Table S4E). When orange fiber (50 mg) was subjected to repeated rounds of extraction with methanol, small quantities of N-methylserotonin were released after each round (31 ng after two rounds; 171 ng in total after 15 rounds; Table S4F). Similar yields were obtained in separate experiments using acetonitrile or acetone (30-31 ng after two rounds of extraction). A comparison of the results of serial methanol extractions of N-methylserotonin against a spike-in compound added to orange fiber whose structure was similar to N-methylserotonin (2-methylserotonin) showed that 95% of 2-methylserotonin was removed by the first cycle of extraction and all of the remaining by the third cycle (Table S4F). These latter observations additionally supported the discovery that N-methylserotonin is “trapped” within orange fiber.

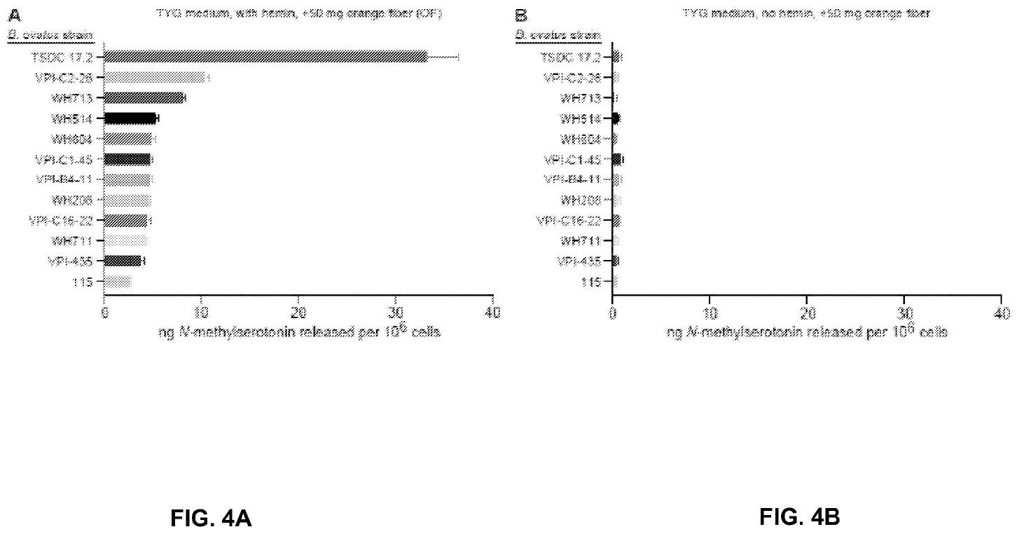

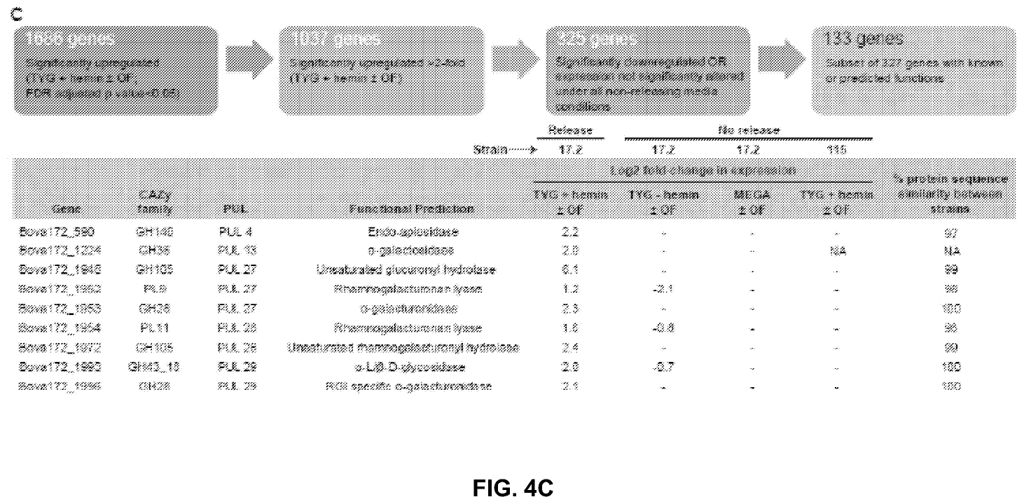

A comparative genomic and functional genomics approach was taken to further characterize the mechanisms underlying release of N-methylserotonin from orange fiber. The N-methylserotonin ‘mining’ activity of B. ovatus TSDC 17.2 was first compared to 11 other human gut-derived strains of B. ovatus. All strains were grown on TYG medium and subjected to the same protocol for assaying N-methylserotonin release from orange fiber as described herein (i.e., 10⁵ input bacterial cells/incubation containing 5 mg/mL orange fiber; 72-hour incubation; 3 replicate assays/strain). Compared to control incubations lacking orange fiber where the yield of N-methylserotonin was 1.5±0.2 ng/10⁶ cells (mean±SD), it was found that all strains were able to release this compound. The amounts released varied between strains, however all strains had mining activities that were significantly lower than TSDC 17.2 (triplicate assays/strain; one-way ANOVA, all P-values<0.0001). B. ovatus 115 had the lowest activity [2.8±0.1 ng (mean±SD) N-methylserotonin released/10⁶ cells compared to 33.3±2.8 ng/10⁶ cells for TSDC 17.2 (FIG. 4A, Table S4G)].

As noted herein, several of the bacterial taxa tested exhibited mining activity that was dependent upon the growth medium used. This observation led to a search for components in TYG whose presence was essential for N-methylserotonin release. This search yielded hemin, a known regulator of gene expression in Bacteroides species. While all strains grew to comparable densities in TYG with or without hemin, no microbe-dependent N-methylserotonin release occurred when these cells were added to reactions containing fresh hemin-deficient TYG plus orange fiber (controls; incubations containing TYG±hemin but lacking orange fiber; FIG. 4B, Table S4H).

To identify the genes likely to contribute to N-methylserotonin release, the genomes of all 12 strains were sequenced, annotated all of their known or predicted encoded proteins, and performed microbial RNA-Seq analysis of gene expression in B. ovatus TSDC 17.2 and B. ovatus 115 grown under conditions identical to those used during assays for N-methylserotonin release activity (72-hour incubation with or without orange fiber in TYG medium with or without hemin, or in MEGA medium). Gene expression in strain TSDC 17.2 under the permissive N-methylserotonin releasing condition (TYG medium±orange fiber) was first compared with gene expression under non-permissive conditions (TYG minus hemin with and without orange fiber). 133 genes were identified that (i) exhibited a statistically significant>1 log 2-fold increase in expression under releasing conditions (Benjamini and Hochberg FDR-adjusted Wald test p-value<0.05), and (ii) were either not significantly differentially expressed (FDR-adjusted p-value>0.1), or were significantly downregulated (FDR-adjusted p-value<0.05) under non-releasing conditions (see Table S6A for a list of these 133 genes plus FIG. 4C).

Natural products are embedded/entrapped in dietary fiber through various chemical and physical interactions. As noted herein, the orange fiber preparation contained nearly 60% (w/w) uronic acid, with prominent representation of homogalacturonan, rhamnogalacturonan, xylan and arabinan structures (Table S1B). Bacteroides species possess multiple polysaccharide utilization loci (PULs). These PULs encode proteins (SusC and SusD homologs) involved in binding and import of various glycan structures as well as carbohydrate active enzymes (CAZymes) that catalyze their degradation [glycoside hydrolases (GH) and polysaccharide lyases (PL)]. Therefore, previously described methods were used to identify PULs and CAZyme gene clusters present in Bacteroides ovatus strains TSDC 17.2 and 115. The results revealed that PUL conservation and synteny between the two strains is very high (Table S7).

Among the 133 genes with statistically significant differential expression in TSDC 17.2, those that manifested the most prominent induction under N-methylserotonin releasing conditions were concentrated in PUL27, PUL28 and PUL29, and to a lesser extent in several other PULs (e.g., PUL4 and PUL13). Proteins encoded by these PULs exhibit>95% amino acid sequence identity with those in strain 115 and share orthologs in the other strains tested (Table S6A, Table S7). Functional assignments for these proteins were made by identifying their best scoring alignments with the sequences of experimentally characterized CAZymes in the CAZy database (www.cazy.org) (Table S6B). The results disclosed members of CAZyme families with reported activities against the backbones of homogalacturonan [GH family 105 (unsaturated rhamnogalacturonyl hydrolase/unsaturated glucuronyl hydrolase)] or rhamnogalacturonan [PL9, PL11 (rhamnogalacturonan lyase); GH28 (RGI-specific a-galacturonidase)]. These structures are prominently represented in pectin and in the orange fiber preparation of the present disclosure (>50% of glycosyl linkages, Table S1B). The PULs also included CAZymes with predicted activities directed at oligosaccharides linked to these backbone structures [arabinofuranosidase (GH43 18), galactosidase (GH36), and apiosidase (GH140)].

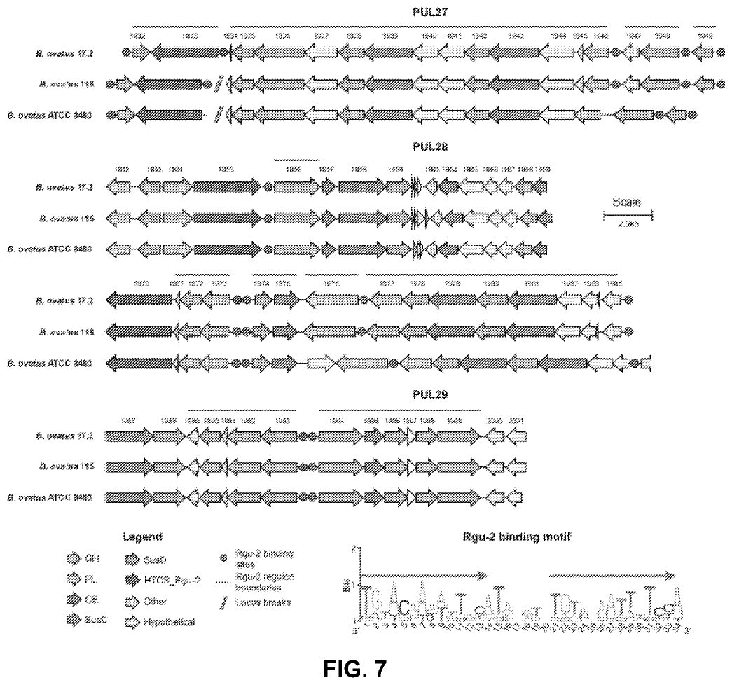

Despite the high degree of PUL conservation between B. ovatus TSDC 17.2 and B. ovatus 115, almost none of their component genes are expressed in the latter strain under mining-permissive conditions (Table S7). In an attempt to define the origin of the observed differences in expression of these PUL genes, and by extrapolation, the discordant N-methylserotonin mining activities of strains TSDC 17.2 and 115, potential transcriptional regulons were reconstructed using comparative genomics (FIG. 7). PUL27, PUL28 and PUL29 form a large chromosomal cluster of 60-70 genes that encode 28 CAZymes, six SusC/SusD transport systems, and three paralogs of a previously characterized rhamnogalacturonan-specific regulator in Bacteroides thetaiotaomicron, HTCS_Rgu-2. Hybrid two-component systems (HTCS) are single polypeptide chains comprised of a transmembrane sensor histidine kinase, a DNA-binding response regulator, and a carbohydrate sensing domain. The reconstructed HTCS_Rgu-2 regulon in B. ovatus strains includes 42 genes from PUL27, PUL28, PUL29, and PUL30, of which 30 were significantly upregulated (FDR-adjusted p-value<0.05) in the presence of orange fiber (Table S7). However, all identified HTCS_Rgu-2 binding sites are highly conserved between the 17.2 and 115 strains, and the orthologous pairs of HTCS regulators are 98-99% identical to each other, indicating (i) conservation of this feature of regulation of rhamnogalacturonan-I utilization loci between the two strains of B. ovatus and (ii) that the observed difference in regulon expression is likely not ascribable to this HTCS alone.

N-methylserotonin levels and microbiome CAZyme gene abundances in humans consuming fiber snack prototypes. To assess the translatability of results obtained from these in vitro analyses and mouse model to humans, two exploratory 10-week open-label, single group assignment studies were performed. The studies involved orange fiber- and pea fiber-supplemented snack food prototypes and dizygotic twins 36.6±2.9 years old (mean±SD) recruited from the Missouri Adolescent Female Twin Study (MOAFTS) cohort. The two studies had the same design (see Methods), with participants supplementing their normal, unrestricted diets with one or the other snack food prototype. In brief, consumption of the fiber snack prototypes escalated from none consumed during the first two weeks, to one snack per day during the third week, then two servings a day during week 4, and finally, beginning week 5, three snacks at which time the maximum daily dose of −25-30 g per day of either pea fiber (Study 1; n=18 participants) or orange fiber (Study 2; n=24 participants, including all 18 from Study 1) was achieved. This dose level was then maintained for 4 weeks (see Table S8A for the composition of the snack prototypes and Table S8B for participant characteristics). Importantly, the orange and pea fiber preparations used for these human studies were obtained from the same commercial sources as those used in preclinical studies. Therefore, analysis of fecal samples collected during the course of these two studies, including from subjects who had participated in both, provided an opportunity to examine the relationship between features of the microbiome and fecal levels of N-methylserotonin as a function of fiber consumption. Specifically, the present disclosure enabled an assessment of whether mining was robust to different background diets, exhibited specificity for orange fiber and was dependent upon the amount of orange fiber consumed.

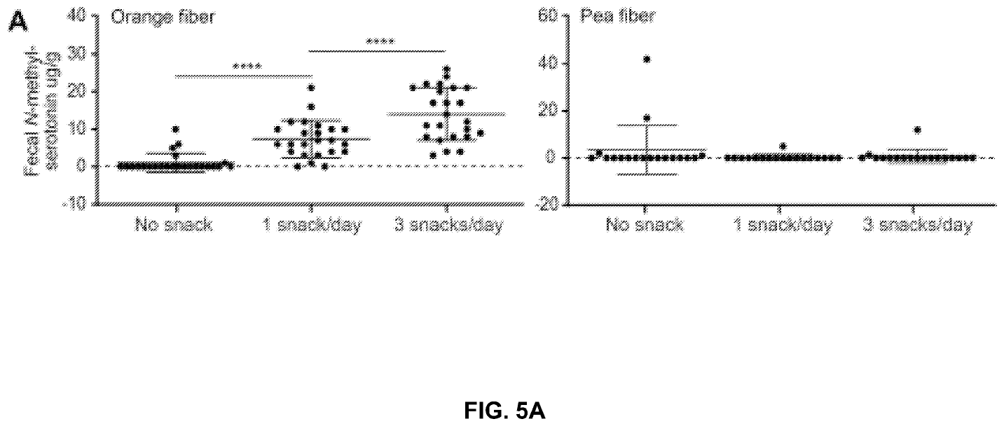

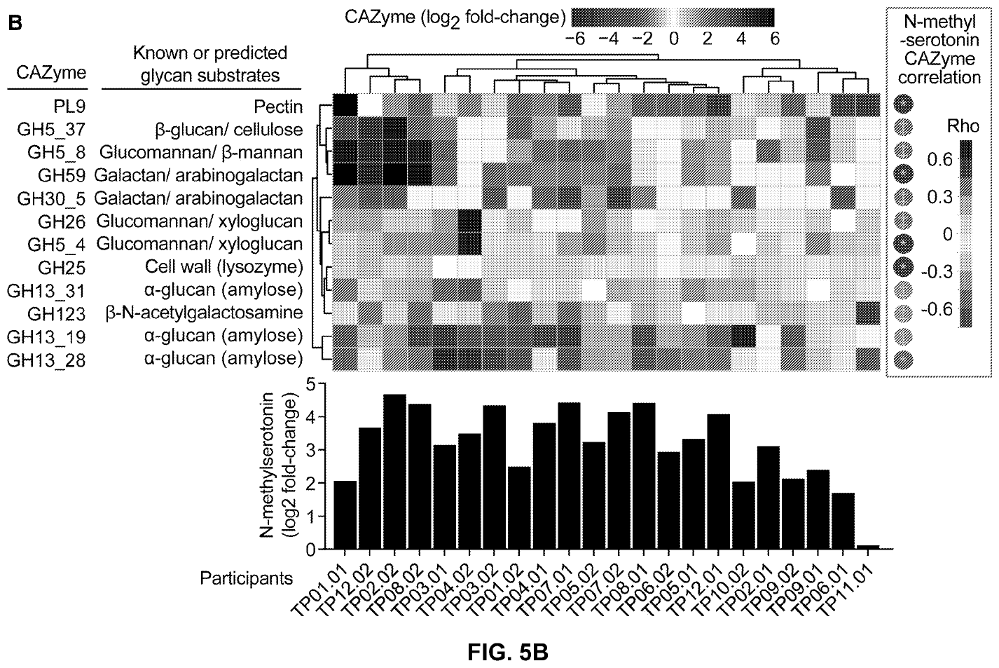

N-methylserotonin it was present in 98% of the 48 samples obtained from participants consuming the orange fiber snack prototype (FIG. 5A and Table S8B). Fecal N-methylserotonin levels were significantly correlated with the number of orange fiber snacks consumed per day (Pearson's r=0.72; p<0.0001) with concentrations reaching 72.5±38.4 μM (mean±SD) at maximal dose. To put this concentration in context, the reported IC50 of binding to a known N-methylserotonin receptor, 5-HT1A, is −2 nM. Fecal serotonin levels were 0-8.6% of that of N-methylserotonin (7±5.7 μM; mean±SD) and did not vary significantly as a function of the dose of orange fiber (Pearson's r=¬0.105, p=0.381; one-way ANOVA p=0.62) (Table S8B). In contrast, N-methylserotonin was undetectable (<0.05 μg/g) in 87% of the 36 fecal samples collected from individuals consuming the pea fiber snack prototype during the supplementation period (at week 3 when one snack per day was being consumed, and at end of week 5, when the maximum dose was being administered) (see the legend to Table S8B regarding the four donors who had positive samples).

Neither the relative abundance of B. ovatus nor of any of the bacterial taxa (Amplicon Sequence Variants, ASVs) that exhibited statistically significant changes in their relative abundances in the fecal microbiota after orange fiber and/or pea fiber snack consumption (Table S8C) had statistically significantly correlations with N-methylserotonin levels at the end of week 5 (Spearman correlation q>0.30) [A statistically significant loge-fold change in relative abundance of a taxon at week 5 compared to the pre-intervention period was defined by q-value<0.1 (linear mixed effect model) and, using higher order singular value decomposition, by positioning of that taxon at the tails (a<0.1) of the distribution of ASVs along tensor component 1; see Methods].

Using shotgun sequencing datasets generated from fecal DNA samples collected at the end of weeks 1 and 5 of the study, a Spearman correlation was performed between (i) the abundances of 213 annotated CAZyme genes [glycoside hydrolases (GH) and polysaccharide lyases (PL)] that were present in at least one study participant at these time points, and (ii) fecal levels of N-methylserotonin prior to fiber supplementation and at the end of week 5 (Table S8D). CAZyme genes whose loge fold-changes in abundance were significantly correlated with levels of N-methylserotonin (q-value<0.1) are shown in FIG. 5B. The strongest positive correlation was with PL9 (rhamnogalacturonan lyase; Spearman rho=0.51, q-value=0.025) (FIG. 5B, Table S8E)—a CAZyme whose expression was significantly upregulated in vitro under mining permissive conditions (loge-fold change 1.2, FDR adjusted p-value (q)=2.2×104, FIG. 4C, Table S6A and Table S7). The CAZyme gene with the second most positive correlation with levels of N-methylserotonin was GH5 37 (Spearman rho=0.438, q=0.08) which has reported specificity for f3-glucan/cellulose. It is notable that in vitro enzymatic digestion experiments of the present disclosure revealed that a preparation enriched in endoglucanases and cellulases exhibited a high level of N-methylserotonin mining activity (Table S4E). Other CAZymes significantly correlated with fecal N-methylserotonin levels included GH30 5 (Spearman rho=0.44, q=0.08) and GH59 (Spearman rho=0.52, q=0.02) which possess homogalacturonan/rhamnogalacturonan processing functions or target pectin components such as galactans and arabinogalactans (FIG. 5B). As was the case with PL9, and GH5 37, the abundances of GH30 5 and GH59 increased significantly in the microbiomes of participants consuming the orange fiber snack (analogous to ASVs, a statistically significant loge-fold change for a CAZyme gene was defined by q-value<0.1 (linear mixed-effects model) and, using higher order singular value decomposition, by the its positioning at the tails (a<0.1) of the distribution of CAZyme genes along tensor component 1). Taken together, the results of the human study of the present disclosure revealed an orange fiber specific, dose-dependent accumulation of N-methylserotonin in feces, where its concentration was positively correlated with the abundances of microbiome genes encoding CAZymes targeting pectic glycans.

Screening of plant sources for N-methylserotonin. Seeking to identify whether the presence of N-methylserotonin is indeed ubiquitous in citrus plants, an array of 23 additional commercially available sources of citrus fibers, sourced from major citrus producing countries across the globe, was screened (Table S9). An orange fiber preparation from Fiberstar, served as control. All but two of the samples tested had significant quantities of N-methylserotonin upon enzymatic digestion, with the ones lacking being either a highly purified/processed pectin product or else a heavily processed unknown citrus fiber blend. The amount of N-methylserotonin present was highly variable between both sample type as well as between individual samples, with some samples being highly variable but approaching control levels (e.g. grounded orange peel, 67±21%) while others having low but more consistent levels (e.g. grounded lemon peel, 3±0.3%). Of particular note is that consistent with the observation of N-methylserotonin being entrapped within orange fiber, the “fine” preparation of the orange fiber used herein (i.e., more heavily refined & processed) yielded much less N-methylserotonin than its less processed counterpart (15±8%).

To further confirm these results, a follow-up screen consisting of locally sourced citrus products were carried out (Table S10). As the orange fiber used herein was described by the manufacturers to be sourced from citrus juice processing, an attempt was made to delineate plausible components of citrus juice processing in the screen. Commonly found edible table orange, lemon, lime, and grapefruits were divided into components labeled “Skin,” “Fruit (with pulp),” “Pulp,” and “Juice” prior to being applied to the same enzymatic screen as described above (Table S10). N-methylserotonin was not found in grapefruit and found in low quantities in the lemon and lime. As expected, it is prominently found in the skin of orange fruits, followed by the fruit, pulp, and juice (161±65%, 122±52%, 35±4%, 36±1%; all relative to the orange fiber used herein). Entrapment of N-methylserotonin is further confirmed by the observation that relative to the enzymatic digestion, very low quantities of N-methylserotonin was released from the same orange peel using non-enzymatic processing methods such as mechanical disruption, methanol extraction, or liquid nitrogen freeze-thawing (Table S10). The presence of N-methylserotonin in the juice is likely due to residue fibrous components remaining in the orange juice during processing, where filtration of orange juice prior to the enzymatic assay is shown to reduce N-methylserotonin levels to negligible levels.

Having confirmed the specificity of N-methylserotonin within citrus fruits and its abundance in oranges, the scope of the screen was broadened to confirm the specificity of plants containing N-methylserotonin. The same enzymatic digestion assay was applied to a broad range of various fruit, vegetable, and grain samples, in total reporting results from 133 different types of edible plants in Table S11(A-E). Samples selected included major global staples such as corn, wheat, rice, and cassava as reported by the Food and Agriculture Organization of the United Nations, as well as commonly consumed fruits and vegetables in America as reported by the USDA and FDA. N-methylserotonin was found in only three sample types, all of which are “peppers” in the Zanthoxylum genus which are in the Rutaceae family alongside citrus fruits. These include two types of the Japanese mountain pepper (Z. piperitum), which is reported to contain N-methylserotonin, and one preparation of the Chinese Sichuan pepper (Z. bungeanum). The otherwise evident absence of N-methylserotonin, including samples belonging to members of the Solanaceae family (various types of hot chili peppers as well as bell peppers) and the Piperaceae family (common black pepper) indicate that this compound is indeed specific to citrus fruits and other members in the Rutaceae family.

### Discussion

Gnotobiotic mice colonized with defined collections of human gut microbes were used together with in vitro assays to show that N-methylserotonin is a compound present in orange fiber that is directly released only by specific members of the gut community to produce significant effects on host physiology and metabolism. A short duration study of a small cohort of adult dizygotic twins disclosed dose-dependent, orange-fiber specific accumulation of N-methylserotonin in their feces. As described herein, orange fiber preparations and their releasable N-methylserotonin are naturally-based analogs of polysaccharide-based drug delivery systems.

Many natural products are embedded in dietary fiber through various chemical and physical interactions, including hydrophobic interactions, hydrogen as well as covalent bonds, and/or physical entrapment. The term “celobiotic” (from the latin ‘conceal or disguise’) is proposed herein to describe a bioactive compound that is liberated from fibers, rather than synthesized or further metabolized, through the actions of one or more microbial enzymes, and whose biological/pharmacologic activities are not dependent upon additional microbial biotransformation.

Several key results aided in deciphering how N-methylserotonin is liberated from orange fiber. A switch was discovered for turning mining activity on and off: Bacteroides ovatus TSDC 17.2, a prominent miner in the preclinical gnotobiotic mouse model described herein, exhibited hemin-dependent release of N-methylserotonin in vitro. Pronounced B. ovatus strain-specific differences in N-methylserotonin releasing activities were documented under in vitro mining permissive conditions; hemin-dependence was a feature of release in all strains. Taking advantage of this hemin-dependency and strain-specificity, microbial RNA-Seq analysis of gene expression in a strong versus weak B. ovatus mining strain incubated in the presence or absence of orange fiber and presence or absence of hemin, revealed a set of glycoside hydrolase and polysaccharide lyase genes associated with release; their known/predicted substrate specificities were consistent with prominent representation of pectic polysaccharides present in orange fiber. Moreover, these in vitro results translated to humans, where treatment with orange and pea fiber snack prototypes disclosed orange fiber-specific accumulation of N-methylserotonin in their feces, with levels of this compound correlating most significantly with the abundances of PL and GH genes involved in processing of glycan structures in pectins. In this respect, and although the specific means of small molecule entrapment differ, it is noteworthy that several members of Bacteroidetes have recently been shown to possess a polysaccharide utilization locus that encodes esterases capable of extracting ferulic acid, a well-documented component of multiple cereal grains.

There have been a limited number of reports describing the biological effects of N-methylserotonin; most of these studies have been conducted in vitro. Similar to serotonin, N-methylserotonin is able to increase glucose uptake in cultured rat muscle via its agonist activity on the 5-HT2A receptor. A maleated form of methylserotonin enhanced insulin secretion in human and mouse beta cells via activation of the 5-HT2B receptor. The closely related compound alpha-methylserotonin, by means of its engagement of 5-HT1 and 5-HT2A, is able to increase glycogen synthesis in rat hepatocytes via a direct increase in glycogen synthase activity as well as cAMP-dependent inactivation of glycogen phosphorylase; binding of serotonin to 5-HT2B/C receptors has an opposing effect and decreases glycogen synthesis.

It was found that oral administration of N-methylserotonin to germ-free mice consuming a high saturated fat, low fiber representative USA diet produced a number of phenotypic changes including reduced adiposity and alterations in hepatic energy (glucose) metabolism. Intriguingly, N-methylserotonin affected expression of regulators of circadian rhythm in both liver and colon, including Arntl, Clock, Pert, Per3, plus Nfil3 and its repressor, Nr1d2. Gut microbiota has been linked to microbiota-regulated diurnal oscillation of epithelial expression of clock components, and the effects of these components (e.g., Nfil3) on lipid absorption and export. RNA-Seq did not reveal significant effects of N-methylserotonin on intestinal or liver levels of mRNAs encoding its known (Htr7, Htr2A) or related (Htr3, Htr4, Htr5 and Htr6) receptors. However, the absence of changes in receptor expression does not preclude effects on their signal transduction pathways, or the possibility that N-methylserotonin exerts its effects on circadian regulators through other metabolites, such as glutamate, whose colonic levels increased after N-methylserotonin administration to germ-free animals.

The ability to manipulate luminal levels of N-methylserotonin in gnotobiotic mice fed orange fiber by including or excluding N-methylserotonin-releasing bacterial species in their gut community illustrates a synbiotic design strategy where fibers containing concealed celobiotics are administered together with probiotic ‘miners’ to enhance/expand the biological effects of fibers to the benefit of the host. For example, administration of free, unbound N-methylserotonin to germ-free mice was found to produce a dose-dependent increase in gastrointestinal transit time, indicating that a synbiotic composed of orange fiber plus a N-methylserotonin miner such as B. ovatus represents an approach for treatment of certain forms of irritable bowel syndrome (IBS-C). Moreover, the systemic effects observed on metabolism in mice exposed to N-methylserotonin indicate the potential for additional beneficial pharmacological properties.

As disclosed herein, celobiotics provide valuable analytic opportunities to both food and microbiome scientists, as well as for synbiotic compositions and therapeutic methods. Liberation of celobiotics from fiber preparations during in vitro incubations of intact uncultured (fecal) microbiota samples, defined consortia of cultured microbes, or single microbial strains operationally define the compositional ‘equivalence’ of different lots of a fiber preparation and/or a comparative assessment of the impact of different food processing methods. As disclosed herein, knowledge of whether a consumer of a fiber preparation harbors a gut microbiota with miners of a specific celobiotic enables analysis of interpersonal variations in responses to that fiber in longer duration clinical studies. A corollary is that more personalized dietary recommendations are enabled by the present disclosure about the types of fiber preparations providing specific health benefits based on a given consumer's known microbiota/microbiome composition.

### Supplemental Tables

Table S1(A-B)—Chemical analysis of fiber preparations; related to FIG. 1(A-C), FIG. 2(A-H), and FIG. 5(A-B). Table S1A Composition of orange and pea fibers. Table S1B Linkage analysis of orange fiber.

Table S2(A-B)—Cecal analytes/features identified by LC-Qtof-MS; related to FIG. 1(A-C). Table S2A Features that are colonization and fiber-dependent; Table S2B Other features. All features shown have peak areas>3-fold higher in colonized mice consuming the HiSF-LoFV+orange fiber diet relative to the other groups. Features are organized by M/Z. Data are presented as peak area.

Table S3(A-D)—Differentially expressed genes in the livers and colons of germ-free mice treated with 50 mg/kg/d N-methylserotonin compared to untreated germ-free controls; related to FIG. 2(A-H). Table S3A Upregulated genes in liver. Table S3B Downregulated genes in liver. Table S3C Upregulated genes in colon. Table S3D Downregulated genes in colon.

Table S4(A-H)—In vitro screening of bacterial strains for N-methylserotonin releasing activity; related to FIG. 1(A-C) and FIG. 4(A-C). Table S4A Levels of N-methylserotonin (ng) released by cultured bacterial strains in TYG with and without hemin, Wilkins-Chalgren anaerobe broth or MEGA medium 2.0 containing orange fiber. Table S4B Screening of additional bacterial strains for N-methylserotonin release from orange fiber. Table S4C Additional control experiments of microbial N-methylserotonin release. Table S4D N-methylserotonin degradation test. Table S4E Release of N-methylserotonin from orange fiber via enzymatic reaction after 72 hours. Table S4F Recovery of N-methylserotonin from orange fiber after repeated rounds of methanol extraction, compared to recovery of a closely related spike-in compound (2-methylserotonin) under the same conditions. Table S4G Screening of additional Bacteroides ovatus strains for N-methylserotonin release from orange fiber using TYG medium with hemin. Table S4H Screening of additional Bacteroides ovatus strains for N-methylserotonin release from orange fiber using TYG medium without hemin. N-methylserotonin levels are shown at the time point specified, tested using a concentration of 5 mg/ml orange fiber, normalized to 10⁶ microbes where applicable, and n=3. ND (not detected)=N-methylserotonin levels below 0.02 ng.

Table S5—Absolute abundances of fecal bacterial community members measured at experimental day 21 (mean±SD); related to FIG. 3(A-G).

Table S6(A-B)—List of “mining” (N-methylserotonin releasing) candidate genes in B. ovatus TSDC 17.2, related to FIG. 4(A-C). Table S6A B. ovatus TSDC 17.2 genes, arranged by log 2 fold-change under the permissive releasing condition (TYG medium containing hemin, with versus without orange fiber). Genes shown exhibit>1 log 2-fold increased expression under the permissive condition, but are not significantly upregulated or downregulated under non-releasing conditions. Highlighted in blue are statistically significant decreases in expression of the same gene under conditions where there is no N-methylserotonin release. Table S6B Functional predictions of CAZymes deemed to be candidate mediators of N-methylserotonin release.

Table S7(A-B)—PUL map of B. ovatus TSDC 17.2; related to FIG. 4(A-C). B. ovatus TSDC 17.2 genes designated as candidates for involvement in N-methylserotonin release from orange fiber (OF) are highlighted in bold font. Table S7A B. ovatus TSDC 17.2; Table S7B Corresponding PUL (if present) in B. ovatus 115. B. ovatus TSDC 17.2 genes designated as candidates for involvement in N-methylserotonin release from orange fiber (OF) are highlighted in bold font and x=present.

Table S8(A-E)-Levels of N-methylserotonin and serotonin in feces collected from adult dizygotic twins consuming orange fiber- or pea fiber-containing snack food prototypes; related to FIG. 5(A-B). Table S8A Composition of the snack food prototypes. Table S8B Ages and BMIs of participants plus levels of N-methylserotonin and serotonin in their fecal samples collected at the end of study weeks 1, 3 and 5. Table S8C ASVs with statistically significant loge-fold changes in relative abundances in the fecal microbiota of participants between the pre-intervention and 5-week time points of the pea and orange fiber snack studies. Table S8D-Week 1 and Table S8D-Week 5 CAZyme (GH and PL) gene representation in the fecal microbiomes of participants consuming orange fiber snacks. Table S8E Spearman correlations of abundances of GH and PL genes and levels of N-methylserotonin in fecal samples collected from study participants at week 5.

Table S9. Assessment of commercially available orange fibers for the presence of N-methylserotonin.

Table S10. Assessment of locally commercially available citrus fruits for the presence of N-methylserotonin.

Table S11(A-E)—N-Methylserotonin screening in other plants, relative to orange fiber (OF). Samples containing N-methylserotonin, where applicable, are mean±SD; “-” indicate not found. Table S11A cash crops and staples. Table S11B common prebiotic supplements. Table S11C spices. Table S11D fruits. Table S11E vegetables.

TABLE S1A
Composition of orange and pea fibers.

| (% dry weight) | Orange Fiber | Pea Fiber |
|---|---|---|
| Σ Total carbohydrates | 82.3% | 77.5% |
| Degree of methylation | 29.0% | 16.0% |
| Protein | 7.0% | 11.6% |
| Lipids | 1.2% | 0.8% |
| Starch | ND | 6.0% |
| Beta-glucans | ND | ND |
| Rhamnose | 1.0% | 0.0% |
| Arabinose | 9.9% | 17.3% |
| Xylose | 2.3% | 4.8% |
| Mannose | 2.7% | 0.5% |
| Galactose | 4.5% | 2.6% |
| Glucose | 17.5% | 38.9% |
| Uronic acids | 45.9% | 13.4% |
| Cutins | 0.5% | 0.0% |
| Methanol | 2.4% | 0.4% |
| Acetic acid | 1.2% | 1.9% |
| Water | 7.2% | 7.4% |

TABLE S1B
Linkage analysis of orange fiber.

| | | | | | | | | | | Common polysaccharide |
|---|---|---|---|---|---|---|---|---|---|---|
| | | %/Σ sugars | | | | | | | | class this residue |
| — | Deduced Linkage | Rha | Fuc | Ara | Xyl | Gal | Glc | Man | UA | arises from |
| Rhamnose | 2-Rha (p) | 0.9 | | | | | | | | RGI |
| Fucose | — | | ND | | | | | | | |
| Arabinose | Terminal Ara (f) | | | 2.4 | | | | | | Type I AG, arabinan |
| | 5-Ara (f) | | | 8.3 | | | | | | Type I AG, arabinan |
| | 3,5-Ara (f) | | | 4.7 | | | | | | Arabinan |
| Xylose | 4-Xyl(p) | | | | 2.4 | | | | | HX |
| Galactose | Terminal Gal(p) | | | | | 0.9 | | | | Galactan, Type I AG |
| | 3-Gal(p) | | | | | 1.2 | | | | Type II AG |
| | 4-Gal(p) | | | | | 8.1 | | | | Galactan, Type I AG |
| | 4,6-Gal(p) | | | | | 0.2 | | | | Type I AG, arabinan |
| Glucose | 4-Glc (p) | | | | | | 13 | | | Cellulose, XG |
| Mannose | — | | | | | | | ND | | |
| Uronic acids | Terminal GalA (p) | | | | | | | | 0.1 | HG, RGI |
| | Terminal GalA (p)-methyl ester | | | | | | | | 0.4 | HG |
| | 4-GalA(p) | | | | | | | | 24 | HG, RGI |
| | 4-GalA(p)-methyl ester | | | | | | | | 31.5 | HG |
| | 3,4-GalA (p) | | | | | | | | 0.4 | |
| | 3,4-GalA (p)-methyl ester | | | | | | | | 0.2 | |
| | 4,6-GalA (p) | | | | | | | | 0.1 | |
| | 4,6-GalA (p)-methyl ester | | | | | | | | 0.4 | |

Abbreviations
Rha Rhamnose
Ara Arabinose
Xyl Xylose
Man Manose
Gal Galactose
Glc Glucose
UA Uronic acids
GalA Galacturonic acid
RGI Rhamnogalacturonan
HG Homogalacturonan
AG Arabinogalactan
XG Xyloglucan
HX Heteroxylan

TABLE S2A
Features that are colonization and fiber-dependent.
Features that are colonization and fiber-dependent

| | | Colonized | | Colonized | |
|---|---|---|---|---|---|
| | Retention time | HiSF-LoFV + | GF HiSF-LoFV + | Unsupplemented | |
| M/Z | (minutes) | orange fiber | orange fiber | HiSF-LoFV | Identification |
| 191.1187 | 2.94 | 126093 | 0 | 0 | N-methylserotonin |
| 362.1999 | 8.94 | 39918 | 0 | 0 | |
| 372.146 | 6.57 | 49611 | 0 | 0 | |
| 385.0447 | 7.12 | 66275 | 0 | 0 | |
| 416.136 | 6.07 | 70732 | 0 | 0 | |
| 416.136 | 6.67 | 34191 | 0 | 0 | |
| 427.0072 | 7.13 | 57878 | 0 | 0 | |
| 452.102 | 6.57 | 89615 | 0 | 0 | |
| 459.2224 | 6.24 | 30015 | 0 | 0 | |
| 462.017 | 7.14 | 51061 | 0 | 0 | |
| 466.1174 | 6.96 | 57883 | 0 | 0 | |
| 473.2188 | 9.48 | 91265 | 0 | 0 | |
| 561.3288 | 5.96 | 37083 | 0 | 0 | |
| 633.3874 | 6.02 | 53216 | 0 | 0 | |

TABLE S2B
Other features.
Other features

| | | Colonized HiSF- | | Colonized | |
|---|---|---|---|---|---|
| | Retention time | LoFV + orange | GF HiSF-LoFV + | Unsupplemented | |
| M/Z | (minutes) | fiber | orange fiber | HiSF-LoFV | Identification |
| 303.0881 | 8.77 | 653253 | 33197 | 0 | Hesperetin |
| 353.0333 | 7.08 | 163730 | 7520 | 0 | Naringenin sulfate |
| 383.0439 | 7.18 | 116293 | 3296 | 0 | Hesperetin sulfate |
| 398.3261 | 10.61 | 55862 | 16344 | 0 | Hexadecenoylcarnitine |
| 154.0589 | 1.25 | 285924 | 55595 | 86823 | |
| 240.1405 | 6.18 | 83570 | 13511 | 8914 | |
| 253.1469 | 6.51 | 83830 | 15353 | 19703 | |
| 253.1503 | 6.4 | 143686 | 39107 | 29235 | |
| 265.1448 | 5.36 | 87821 | 18988 | 27784 | |
| 268.1063 | 1.67 | 414629 | 30915 | 119699 | |
| 271.1535 | 5.76 | 129903 | 0 | 21094 | |
| 271.1542 | 5.8 | 112784 | 2994 | 20537 | |
| 271.1558 | 7.04 | 37119 | 9591 | 6198 | |
| 276.1201 | 1.71 | 1721637 | 6643 | 439800 | |
| 280.159 | 5.79 | 93434 | 17155 | 11714 | |
| 284.1973 | 4.78 | 145340 | 5417 | 12834 | |
| 285.2431 | 10.99 | 53598 | 9520 | 4230 | |
| 287.0922 | 10.35 | 108454 | 6089 | 11566 | |
| 287.1588 | 5.59 | 147929 | 38778 | 45858 | |
| 289.1808 | 7.08 | 42376 | 13769 | 5097 | |
| 295.1553 | 5.31 | 75646 | 24009 | 15179 | |
| 297.2429 | 9.55 | 44589 | 0 | 4434 | |
| 297.243 | 9.38 | 52193 | 0 | 4866 | |
| 303.0892 | 7.08 | 619060 | 12697 | 0 | |
| 304.1621 | 7.15 | 139852 | 16075 | 16252 | |
| 308.1202 | 4.63 | 162217 | 50196 | 18537 | |
| 313.1629 | 6.2 | 62702 | 20405 | 16001 | |
| 313.2372 | 8.29 | 132185 | 9874 | 21059 | |
| 313.238 | 8.32 | 93281 | 11245 | 18074 | |
| 316.2142 | 6 | 333612 | 57620 | 66879 | |
| 316.2856 | 11.87 | 264433 | 54276 | 81113 | |
| 316.286 | 12.13 | 857988 | 174344 | 268335 | |
| 322.1714 | 5.92 | 183641 | 57724 | 17668 | |
| 327.2176 | 9.8 | 78940 | 22969 | 11407 | |
| 329.1039 | 7.29 | 182043 | 26277 | 0 | |
| 347.255 | 5.78 | 64959 | 15778 | 8203 | |
| 348.109 | 4.44 | 141424 | 20630 | 0 | |
| 352.1817 | 5.58 | 165391 | 33146 | 13686 | |
| 356.2158 | 4.72 | 66240 | 0 | 19774 | |
| 359.1139 | 7.29 | 519635 | 56376 | 0 | |
| 360.207 | 5.3 | 71199 | 21948 | 22669 | |
| 361.1807 | 4.87 | 60564 | 3040 | 0 | |
| 362.2024 | 7.42 | 91859 | 29659 | 14438 | |
| 366.136 | 6.41 | 65745 | 15341 | 0 | |
| 371.2576 | 8.94 | 76319 | 12736 | 23289 | |
| 384.2739 | 7.08 | 29271 | 8144 | 0 | |
| 386.2284 | 4.64 | 270639 | 0 | 85241 | |
| 390.2104 | 5.85 | 68326 | 6137 | 12222 | |
| 390.2176 | 5.14 | 94499 | 0 | 12828 | |
| 390.2309 | 8.25 | 43276 | 13770 | 10811 | |
| 400.3061 | 11.12 | 76982 | 16169 | 15728 | |
| 408.2423 | 10.38 | 64731 | 7437 | 8812 | |
| 413.1975 | 10.39 | 132575 | 18385 | 30995 | |
| 414.3342 | 8.7 | 90818 | 0 | 16476 | |
| 416.2622 | 7.1 | 262479 | 38649 | 64978 | |
| 416.266 | 6.64 | 1032373 | 316823 | 238432 | |
| 416.3374 | 8.34 | 88053 | 29190 | 15039 | |
| 416.3376 | 8.19 | 140019 | 44493 | 15092 | |
| 416.3381 | 13.54 | 3673802 | 128674 | 1047035 | |
| 416.3384 | 8.26 | 293163 | 87557 | 15092 | |
| 418.2815 | 6.23 | 51162 | 16666 | 8436 | |
| 424.2304 | 6.73 | 41207 | 10727 | 6735 | |
| 424.2833 | 13.11 | 228675 | 4902 | 66726 | |
| 428.2178 | 7.41 | 46219 | 13548 | 15207 | |
| 430.1563 | 5.88 | 109065 | 24400 | 10656 | |
| 430.2279 | 8.98 | 107917 | 28866 | 0 | |
| 432.2444 | 8.28 | 180421 | 43049 | 55334 | |
| 433.1167 | 5.86 | 200856 | 13844 | 14927 | |
| 434.2947 | 11.32 | 86358 | 12150 | 15517 | |
| 434.2953 | 11.69 | 78959 | 10275 | 16829 | |
| 435.2836 | 7.2 | 43996 | 11764 | 14549 | |
| 436.2834 | 13.54 | 148199 | 7477 | 31615 | |
| 442.1993 | 5.26 | 198413 | 5923 | 30597 | |
| 444.2972 | 9.48 | 516043 | 145371 | 55158 | |
| 448.2896 | 7.11 | 53826 | 0 | 16265 | |
| 451.272 | 10.71 | 60871 | 13386 | 0 | |
| 454.2939 | 13.31 | 1813060 | 72271 | 440992 | |
| 454.2945 | 12.99 | 111703 | 9139 | 34678 | |
| 468.3097 | 14.36 | 341429 | 12757 | 89548 | |
| 468.3101 | 14.07 | 438081 | 5354 | 91425 | |
| 471.2063 | 6.58 | 61178 | 0 | 7272 | |
| 478.2946 | 7.21 | 88681 | 11665 | 18210 | |
| 479.2764 | 6.38 | 46904 | 7319 | 5904 | |
| 496.0843 | 6.11 | 117930 | 4635 | 0 | |
| 497.1598 | 5.55 | 102021 | 3899 | 0 | |
| 503.242 | 5.93 | 38680 | 7406 | 0 | |
| 507.3073 | 7.31 | 94446 | 25250 | 24542 | |
| 507.3104 | 7.42 | 423728 | 102426 | 39255 | |
| 515.2319 | 10.33 | 104922 | 13850 | 0 | |
| 522.3568 | 13.9 | 766992 | 164756 | 56170 | |
| 533.2412 | 9.84 | 125735 | 16157 | 0 | |
| 533.2425 | 7.67 | 108790 | 29999 | 0 | |
| 539.429 | 13.4 | 82018 | 22776 | 25728 | |
| 545.2883 | 7.25 | 107950 | 33428 | 35827 | |
| 547.3064 | 8.04 | 479215 | 126453 | 48276 | |
| 547.3067 | 8.16 | 579324 | 174829 | 45010 | |
| 547.3076 | 8.96 | 223571 | 71979 | 51845 | |
| 549.3239 | 8.84 | 285264 | 90939 | 31799 | |
| 579.4234 | 11.78 | 57330 | 4179 | 17489 | |
| 619.4081 | 7.32 | 31945 | 0 | 7871 | |
| 653.1755 | 6.57 | 54859 | 14085 | 0 | |
| 1079.6506 | 6.29 | 36667 | 9593 | 12101 | |

TABLE S3A
Upregulated genes in liver.

| | | | | FDR- |
|---|---|---|---|---|
| | | | | adjusted |
| Entrez | | | log2 fold- | Wald test |
| Gene ID | Symbol | Name | change | P-value |
| 13170 | Dbp | D site albumin promoter binding protein | 3.42 | 3.73E−133 |
| 21685 | Tef | thyrotroph embryonic factor | 1.74 | 7.51E−43 |
| 229599 | Ciart | circadian associated repressor of transcription | 5.11 | 6.68E−35 |
| 18628 | Per3 | period circadian clock 3 | 3.15 | 1.35E−29 |
| 53376 | Usp2 | ubiquitin specific peptidase 2 | 3.50 | 5.30E−29 |
| 18551 | Pcsk4 | proprotein convertase subtilisin/kexin type 4 | 2.53 | 2.34E−22 |
| 20893 | Bhlhe40 | basic helix-loop-helix family, member e40 | 1.84 | 7.40E−22 |
| 76654 | Upp2 | uridine phosphorylase 2 | 2.11 | 3.23E−21 |
| 15379 | Onecut1 | one cut domain, family member 1 | 2.42 | 2.00E−19 |
| 353187 | Nr1d2 | nuclear receptor subfamily 1, group D, member 2 | 1.07 | 1.22E−18 |
| 67608 | Narf | nuclear prelamin A recognition factor | 0.95 | 7.17E−18 |
| 79362 | Bhlhe41 | basic helix-loop-helix family, member e41 | 2.98 | 1.89E−16 |
| 22390 | Wee1 | WEE 1 homolog 1 (S. pombe) | 2.59 | 1.89E−14 |
| 80290 | Gpr146 | G protein-coupled receptor 146 | 1.09 | 8.31E−14 |
| 72401 | Slc43a1 | solute carrier family 43, member 1 | 1.14 | 1.24E−12 |
| 94071 | Clec2h | C-type lectin domain family 2, member h | 1.31 | 2.22E−12 |
| 74182 | Gpcpd1 | glycerophosphocholine phosphodiesterase 1 | 1.25 | 2.28E−12 |
| 26399 | Map2k6 | mitogen-activated protein kinase kinase 6 | 1.53 | 1.10E−10 |
| 18984 | Por | cytochrome p450 oxidoreductase | 1.24 | 1.12E−10 |
| 211401 | Mtss1 | MTSS I-BAR domain containing 1 | 1.00 | 1.15E−10 |
| 50794 | Klf13 | Kruppel-like factor 13 | 1.35 | 1.35E−10 |
| 12013 | Bach1 | BTB and CNC homology 1, basic leucine zipper | 0.70 | 1.01E−09 |
| | | transcription factor 1 | | |
| 78894 | Aacs | acetoacetyl-CoA synthetase | 2.50 | 2.51E−09 |
| 171543 | Bmf | BCL2 modifying factor | 1.43 | 2.66E−09 |
| 23893 | Grem2 | gremlin 2, DAN family BMP antagonist | 1.12 | 3.06E−09 |
| 56485 | Slc2a5 | solute carrier family 2 (facilitated glucose | 1.38 | 8.22E−09 |
| | | transporter), member 5 | | |
| 16601 | Klf9 | Kruppel-like factor 9 | 0.72 | 1.29E−08 |
| 170459 | Stard4 | StAR-related lipid transfer (START) domain | 0.72 | 1.37E−08 |
| | | containing 4 | | |
| 12606 | Cebpa | CCAAT/enhancer binding protein (C/EBP), alpha | 0.50 | 1.66E−08 |
| 216742 | Fnip1 | folliculin interacting protein 1 | 0.43 | 2.73E−08 |
| 52331 | Stbd1 | starch binding domain 1 | 1.04 | 3.36E−08 |
| 11655 | Alas1 | aminolevulinic acid synthase 1 | 1.38 | 1.22E−07 |
| 214253 | Etnk2 | ethanolamine kinase 2 | 0.72 | 1.56E−07 |
| 13087 | Cyp2a5 | cytochrome P450, family 2, subfamily a, | 1.17 | 2.01E−07 |
| | | polypeptide 5 | | |
| 20733 | Spint2 | serine protease inhibitor, Kunitz type 2 | 0.92 | 2.37E−07 |
| 21847 | Klf10 | Kruppel-like factor 10 | 1.52 | 2.47E−07 |
| 170826 | Ppargc1b | peroxisome proliferative activated receptor, | | |
| | | gamma, coactivator 1 beta | 1.35 | 2.47E−07 |
| 56338 | Txnip | thioredoxin interacting protein | 1.17 | 2.59E−07 |
| 433022 | Plcxd2 | phosphatidylinositol-specific phospholipase C, X | 0.50 | 6.20E−07 |
| | | domain containing 2 | | |
| 16169 | Il15ra | interleukin 15 receptor, alpha chain | 0.79 | 6.62E−07 |
| 11819 | Nr2f2 | nuclear receptor subfamily 2, group F, member 2 | 0.53 | 9.93E−07 |
| 232493 | Gys2 | glycogen synthase 2 | 0.69 | 1.21E−06 |
| 110855 | Pde6c | phosphodiesterase 6C, cGMP specific, cone, alpha | 2.38 | 1.45E−06 |
| | | prime | | |
| 330064 | Slc5a6 | solute carrier family 5 (sodium-dependent vitamin | 1.32 | 1.62E−06 |
| | | transporter), member 6 | | |
| 319476 | Lrtm1 | leucine-rich repeats and transmembrane domains 1 | 0.73 | 2.03E−06 |
| 27984 | Efhd2 | EF hand domain containing 2 | 0.85 | 2.03E−06 |
| 622434 | Arhgef26 | Rho guanine nucleotide exchange factor (GEF) 26 | 1.10 | 2.11E−06 |
| 18441 | P2ry1 | purinergic receptor P2Y, G-protein coupled 1 | 1.00 | 2.23E−06 |
| 69053 | 1810013L24Rik | RIKEN cDNA 1810013L24 gene | 0.54 | 2.59E−06 |
| 53972 | Ngef | neuronal guanine nucleotide exchange factor | 0.76 | 3.25E−06 |
| 67432 | Hoga1 | 4-hydroxy-2-oxoglutarate aldolase 1 | 0.44 | 3.27E−06 |
| 76560 | Prss8 | protease, serine 8 (prostasin) | 1.32 | 3.67E−06 |
| 235320 | Zbtb16 | zinc finger and BTB domain containing 16 | 1.45 | 4.56E−06 |
| 100637 | N4bp2l1 | NEDD4 binding protein 2-like 1 | 0.61 | 4.98E−06 |
| 76454 | Fbxo31 | F-box protein 31 | 0.74 | 6.06E−06 |
| 107569 | Nt5c3 | 5′-nucleotidase, cytosolic III | 0.52 | 9.28E−06 |
| 67333 | Stk35 | serine/threonine kinase 35 | 0.74 | 9.89E−06 |
| 140742 | Sesn1 | sestrin 1 | 0.69 | 9.91E−06 |
| 27528 | Nrep | neuronal regeneration related protein | 1.42 | 1.17E−05 |
| 66270 | Retreg1 | reticulophagy regulator 1 | 0.64 | 1.21E−05 |
| 18627 | Per2 | period circadian clock 2 | 1.23 | 1.23E−05 |
| 15936 | Ier2 | immediate early response 2 | 0.89 | 1.26E−05 |
| 242785 | Klhl21 | kelch-like 21 | 0.81 | 1.44E−05 |
| 64291 | Osbpl1a | oxysterol binding protein-like 1A | 0.37 | 1.44E−05 |
| 67379 | Dedd2 | death effector domain-containing DNA binding | 0.83 | 1.72E−05 |
| | | protein 2 | | |
| 13108 | Cyp2g1 | cytochrome P450, family 2, subfamily g, | 1.58 | 1.75E−05 |
| | | polypeptide 1 | | |
| 66898 | Baiap2l1 | BAl1-associated protein 2-like 1 | 0.74 | 1.84E−05 |
| 70024 | Mcm10 | minichromosome maintenance 10 replication | 0.73 | 2.37E−05 |
| | | initiation factor | | |
| 54375 | Azin1 | antizyme inhibitor 1 | 0.59 | 2.41E−05 |
| 72611 | Zfp655 | zinc finger protein 655 | 0.73 | 2.95E−05 |
| 74043 | Pex26 | peroxisomal biogenesis factor 26 | 0.59 | 3.51E−05 |
| 22339 | Vegfa | vascular endothelial growth factor A | 0.51 | 3.54E−05 |
| 13875 | Erf | Ets2 repressor factor | 0.68 | 3.57E−05 |
| 102032 | Smim19 | small integral membrane protein 19 | 0.61 | 4.61E−05 |
| 108682 | Gpt2 | glutamic pyruvate transaminase (alanine | 0.38 | 4.72E−05 |
| | | aminotransferase) 2 | | |
| 66626 | Cdip1 | cell death inducing Trp53 target 1 | 0.54 | 5.79E−05 |
| 269424 | Jade1 | jade family PHD finger 1 | 0.43 | 6.07E−05 |
| 235047 | Zfp809 | zinc finger protein 809 | 0.73 | 6.08E−05 |
| 24057 | Sh3yl1 | Sh3 domain YSC-like 1 | 1.25 | 6.33E−05 |
| 59027 | Nampt | nicotinamide phosphoribosyltransferase | 0.51 | 6.47E−05 |
| 75234 | Rnf19b | ring finger protein 19B | 0.54 | 6.81E−05 |
| 13835 | Epha1 | Eph receptor A1 | 0.63 | 7.69E−05 |
| 66333 | Aqp11 | aquaporin 11 | 0.61 | 8.33E−05 |
| 50709 | H1f4 | H1.4 linker histone, cluster member | 0.49 | 8.53E−05 |
| 223775 | Pim3 | proviral integration site 3 | 0.72 | 1.08E−04 |
| 230789 | Fam76a | family with sequence similarity 76, member A | 0.59 | 1.18E−04 |
| 67876 | Coq10b | coenzyme Q10B | 1.00 | 1.30E−04 |
| 228775 | Trib3 | tribbles pseudokinase 3 | 1.00 | 1.58E−04 |
| 20055 | Rps16 | ribosomal protein S16 | 2.18 | 1.58E−04 |
| 20515 | Slc20a1 | solute carrier family 20, member 1 | 0.89 | 1.69E−04 |
| 16477 | Junb | jun B proto-oncogene | 1.69 | 1.89E−04 |
| 73212 | 3110082l17Rik | RIKEN cDNA 3110082l17 gene | 1.21 | 1.98E−04 |
| 329679 | Fnip2 | folliculin interacting protein 2 | 0.66 | 2.00E−04 |
| 16890 | Lipe | lipase, hormone sensitive | 0.70 | 2.09E−04 |
| 252838 | Tox | thymocyte selection-associated high mobility group | 1.31 | 2.18E−04 |
| | | box | | |
| 432720 | Akr1c19 | aldo-keto reductase family 1, member C19 | 0.49 | 2.24E−04 |
| 270672 | Map3k15 | mitogen-activated protein kinase kinase kinase 15 | 1.42 | 2.27E−04 |
| 70012 | Cep85 | centrosomal protein 85 | 0.48 | 2.28E−04 |
| 13112 | Cyp3a11 | cytochrome P450, family 3, subfamily a, | 0.60 | 2.38E−04 |
| | | polypeptide 11 | | |
| 19652 | Rbm3 | RNA binding motif (RNP1, RRM) protein 3 | 0.68 | 2.57E−04 |
| 16840 | Cnmd | chondromodulin | 0.88 | 3.12E−04 |
| 110198 | Akr7a5 | aldo-keto reductase family 7, member A5 (aflatoxin | 0.52 | 3.15E−04 |
| | | aldehyde reductase) | | |
| 93679 | Trim8 | tripartite motif-containing 8 | 0.50 | 3.69E−04 |
| 71795 | Pitpnc1 | phosphatidylinositol transfer protein, cytoplasmic 1 | 0.51 | 3.73E−04 |
| 56282 | Mrpl12 | mitochondrial ribosomal protein L12 | 1.04 | 4.12E−04 |
| 215418 | Csrnp1 | cysteine-serine-rich nuclear protein 1 | 1.22 | 4.58E−04 |
| 225049 | Ttc7 | tetratricopeptide repeat domain 7 | 0.50 | 4.90E−04 |
| 17133 | Maff | v-maf musculoaponeurotic fibrosarcoma oncogene | 1.06 | 4.94E−04 |
| | | family, protein F (avian) | | |
| 28010 | Miip | migration and invasion inhibitory protein | 0.66 | 5.27E−04 |
| 116701 | Fgfrl1 | fibroblast growth factor receptor-like 1 | 0.63 | 5.29E−04 |
| 11639 | Ak4 | adenylate kinase 4 | 0.52 | 5.40E−04 |
| 71207 | Nudt4 | nudix (nucleoside diphosphate linked moiety X)- | 0.47 | 5.51E−04 |
| | | type motif 4 | | |
| 66840 | Wdr45b | WD repeat domain 45B | 0.74 | 6.49E−04 |
| 14872 | Gstt2 | glutathione S-transferase, theta 2 | 0.61 | 7.34E−04 |
| 231717 | Pheta1 | PH domain containing endocytic trafficking adaptor | 0.88 | 7.79E−04 |
| | | 1 | | |
| 15081 | H3f3b | H3.3 histone B | 0.36 | 8.26E−04 |
| 240638 | Slc16a12 | solute carrier family 16 (monocarboxylic acid | 0.54 | 8.26E−04 |
| | | transporters), member 12 | | |
| 97112 | Nmd3 | NMD3 ribosome export adaptor | 0.50 | 9.12E−04 |
| 216363 | Rab3ip | RAB3A interacting protein | 0.37 | 9.75E−04 |
| 74533 | Gzf1 | GDNF-inducible zinc finger protein 1 | 0.38 | 1.05E−03 |
| 210998 | Fam91a1 | family with sequence similarity 91, member A1 | 0.33 | 1.26E−03 |
| 56551 | Txn2 | thioredoxin 2 | 0.89 | 1.33E−03 |
| 56336 | B4galt5 | UDP-Gal: betaGlcNAc beta 1,4- | 0.68 | 1.37E−03 |
| | | galactosyltransferase, polypeptide 5 | | |
| 21743 | Inmt | indolethylamine N-methyltransferase | 0.52 | 1.54E−03 |
| 26397 | Map2k3 | mitogen-activated protein kinase kinase 3 | 0.60 | 1.65E−03 |
| 230088 | Fam214b | family with sequence similarity 214, member B | 0.71 | 1.70E−03 |
| 209039 | Tns2 | tensin 2 | 0.65 | 1.71E−03 |
| 245038 | Dclk3 | doublecortin-like kinase 3 | 0.65 | 1.74E−03 |
| 77053 | Sun1 | Sad1 and UNC84 domain containing 1 | 0.52 | 1.76E−03 |
| 12371 | Casp9 | caspase 9 | 0.54 | 1.76E−03 |
| 112406 | Egln2 | egl-9 family hypoxia-inducible factor 2 | 0.42 | 1.83E−03 |
| 14012 | Mpzl2 | myelin protein zero-like 2 | 0.65 | 1.94E−03 |
| 230590 | Zyg11a | zyg-11 family member A, cell cycle regulator | 0.83 | 1.94E−03 |
| 12193 | Zfp36l2 | zinc finger protein 36, C3H type-like 2 | 0.79 | 1.96E−03 |
| 207278 | Fchsd2 | FCH and double SH3 domains 2 | 0.65 | 2.19E−03 |
| 108168395 | Gm45871 | predicted gene 45871 | 0.86 | 2.22E−03 |
| 109263 | Rlf | rearranged L-myc fusion sequence | 0.41 | 2.25E−03 |
| 17347 | Mknk2 | MAP kinase-interacting serine/threonine kinase 2 | 0.79 | 2.27E−03 |
| 67705 | 1810058l24Rik | RIKEN cDNA 1810058l24 gene | 0.53 | 2.32E−03 |
| 100038525 | Gm10804 | predicted gene 10804 | 1.19 | 2.32E−03 |
| 14528 | Gch1 | GTP cyclohydrolase 1 | 0.44 | 2.46E−03 |
| 229473 | Tmem131l | transmembrane 131 like | 0.51 | 2.52E−03 |
| 20454 | St3gal5 | ST3 beta-galactoside alpha-2,3-sialyltransferase 5 | 0.79 | 2.60E−03 |
| 69718 | Ipmk | inositol polyphosphate multikinase | 0.34 | 2.60E−03 |
| 77006 | Ddrgk1 | DDRGK domain containing 1 | 0.33 | 2.62E−03 |
| 12176 | Bnip3 | BCL2/adenovirus E1B interacting protein 3 | 0.57 | 2.69E−03 |
| 320024 | Nceh1 | neutral cholesterol ester hydrolase 1 | 0.50 | 2.69E−03 |
| 19734 | Rgs16 | regulator of G-protein signaling 16 | 2.44 | 2.83E−03 |
| 217166 | Nr1d1 | nuclear receptor subfamily 1, group D, member 1 | 0.66 | 2.94E−03 |
| 76487 | Ppp1r3g | protein phosphatase 1, regulatory subunit 3G | 2.55 | 3.01E−03 |
| 14229 | Fkbp5 | FK506 binding protein 5 | 0.75 | 3.02E−03 |
| 213990 | Agap3 | ArfGAP with GTPase domain, ankyrin repeat and | 0.38 | 3.06E−03 |
| | | PH domain 3 | | |
| 72244 | 1600014C10Rik | RIKEN cDNA 1600014C10 gene | 0.27 | 3.10E−03 |
| 74519 | Cyp2j9 | cytochrome P450, family 2, subfamily j, polypeptide | 0.74 | 3.14E−03 |
| | | 9 | | |
| 16010 | Igfbp4 | insulin-like growth factor binding protein 4 | 0.25 | 3.16E−03 |
| 15446 | Hpgd | hydroxyprostaglandin dehydrogenase 15 (NAD) | 0.50 | 3.17E−03 |
| 27376 | Slc25a10 | solute carrier family 25 (mitochondrial carrier, | 0.32 | 3.25E−03 |
| | | dicarboxylate transporter), member 10 | | |
| 244421 | Lonrf1 | LON peptidase N-terminal domain and ring finger 1 | 0.89 | 3.39E−03 |
| 170460 | Stard5 | StAR-related lipid transfer (START) domain | 0.38 | 3.49E−03 |
| | | containing 5 | | |
| 77252 | 9430038101Rik | RIKEN cDNA 9430038l01 gene | 0.73 | 3.69E−03 |
| 16177 | Il1r1 | interleukin 1 receptor, type I | 0.45 | 3.69E−03 |
| 72056 | 1810055G02Rik | RIKEN cDNA 1810055G02 gene | 0.50 | 3.82E−03 |
| 102462 | Imp3 | IMP3, U3 small nucleolar ribonucleoprotein | 0.49 | 3.94E−03 |
| 72399 | Brap | BRCA1 associated protein | 0.31 | 3.94E−03 |
| 50798 | Gne | glucosamine (UDP-N-acetyl)-2-epimerase/N- | 0.40 | 3.97E−03 |
| | | acetylmannosamine kinase | | |
| 17937 | Nab2 | Ngfi-A binding protein 2 | 0.76 | 3.98E−03 |
| 12034 | Phb2 | prohibitin 2 | 0.30 | 3.99E−03 |
| 383295 | Ypel5 | yippee like 5 | 0.42 | 4.21E−03 |
| 546071 | Mast3 | microtubule associated serine/threonine kinase 3 | 0.75 | 4.60E−03 |
| 171281 | Acot3 | acyl-CoA thioesterase 3 | 1.49 | 5.03E−03 |
| 18141 | Nup50 | nucleoporin 50 | 0.44 | 5.15E−03 |
| 14894 | Cfap20 | cilia and flagella associated protein 20 | 0.41 | 5.38E−03 |
| 56508 | Rapgef4 | Rap guanine nucleotide exchange factor (GEF) 4 | 0.61 | 5.41E−03 |
| 225341 | Lims2 | LIM and senescent cell antigen like domains 2 | 0.35 | 5.41E−03 |
| 320717 | Pptc7 | PTC7 protein phosphatase homolog | 0.43 | 5.41E−03 |
| 18286 | Odf2 | outer dense fiber of sperm tails 2 | 0.53 | 5.72E−03 |
| 55942 | Sertad1 | SERTA domain containing 1 | 0.78 | 5.94E−03 |
| 333433 | Gpd1l | glycerol-3-phosphate dehydrogenase 1-like | 0.35 | 6.09E−03 |
| 24055 | Sh3bp2 | SH3-domain binding protein 2 | 0.56 | 6.55E−03 |
| 22793 | Zyx | zyxin | 0.62 | 6.55E−03 |
| 99929 | Tiparp | TCDD-inducible poly(ADP-ribose) polymerase | 0.45 | 6.77E−03 |
| 67118 | Bfar | bifunctional apoptosis regulator | 0.33 | 6.95E−03 |
| 50880 | Scly | selenocysteine lyase | 0.34 | 7.07E−03 |
| 330323 | Mindy4 | MINDY lysine 48 deubiquitinase 4 | 0.78 | 7.12E−03 |
| 100039795 | Ildr2 | immunoglobulin-like domain containing receptor 2 | 0.44 | 7.27E−03 |
| 76073 | Pcgf5 | polycomb group ring finger 5 | 0.36 | 7.48E−03 |
| 14066 | F3 | coagulation factor III | 0.80 | 7.49E−03 |
| 22433 | Xbp1 | X-box binding protein 1 | 0.60 | 7.52E−03 |
| 67803 | Limd2 | LIM domain containing 2 | 0.51 | 7.60E−03 |
| 24066 | Spry4 | sprouty RTK signaling antagonist 4 | 0.63 | 7.64E−03 |
| 22340 | Vegfb | vascular endothelial growth factor B | 0.80 | 7.76E−03 |
| 68778 | Gucd1 | guanylyl cyclase domain containing 1 | 0.38 | 7.76E−03 |
| 210172 | Zfp526 | zinc finger protein 526 | 0.68 | 8.01E−03 |
| 22282 | Usf2 | upstream transcription factor 2 | 0.34 | 8.18E−03 |
| 105387 | Akr1c14 | aldo-keto reductase family 1, member C14 | 0.49 | 8.18E−03 |
| 94091 | Trim11 | tripartite motif-containing 11 | 0.48 | 8.24E−03 |
| 58249 | Fibp | fibroblast growth factor (acidic) intracellular binding | 0.52 | 8.31E−03 |
| | | protein | | |
| 67804 | Snx2 | sorting nexin 2 | 0.30 | 8.70E−03 |
| 106585 | Ankrd12 | ankyrin repeat domain 12 | 0.74 | 8.71E−03 |
| 22156 | Tuft1 | tuftelin 1 | 0.84 | 8.90E−03 |
| 11637 | Ak2 | adenylate kinase 2 | 0.26 | 8.97E−03 |
| 11735 | Ank3 | ankyrin 3, epithelial | 0.55 | 9.01E−03 |
| 628308 | Zfp970 | zinc finger protein 970 | 0.83 | 9.23E−03 |
| 228355 | Madd | MAP-kinase activating death domain | 0.41 | 9.33E−03 |
| 241694 | Ralgapa2 | Ral GTPase activating protein, alpha subunit 2 | 0.35 | 9.33E−03 |
| | | (catalytic) | | |
| 210009 | Mtrr | 5-methyltetrahydrofolate-homocysteine | 0.54 | 9.33E−03 |
| | | methyltransferase reductase | | |
| 70237 | Bhlhb9 | basic helix-loop-helix domain containing, class B9 | 0.85 | 9.33E−03 |
| 15451 | Hpn | hepsin | 0.32 | 9.45E−03 |
| 104923 | Adi1 | acireductone dioxygenase 1 | 0.30 | 9.54E−03 |
| 97122 | H4c14 | H4 clustered histone 14 | 0.60 | 9.56E−03 |
| 245945 | Rbm47 | RNA binding motif protein 47 | 0.49 | 9.76E−03 |
| 52670 | Cpsf4l | cleavage and polyadenylation specific factor 4-like | 0.68 | 9.86E−03 |
| 242721 | Klhdc7a | kelch domain containing 7A | 0.42 | 9.92E−03 |
| 106393 | Srl | sarcalumenin | 1.27 | 1.00E−02 |
| 50926 | Hnrnpdl | heterogeneous nuclear ribonucleoprotein D-like | 0.35 | 1.00E−02 |
| 14685 | Gnat1 | guanine nucleotide binding protein, alpha | 0.63 | 1.01E−02 |
| | | transducing 1 | | |
| 269608 | Plekhg5 | pleckstrin homology domain containing, family G | 0.88 | 1.02E−02 |
| | | (with RhoGef domain) member 5 | | |
| 235323 | Usp28 | ubiquitin specific peptidase 28 | 0.58 | 1.02E−02 |
| 217708 | Lin52 | lin-52 homolog (C. elegans) | 0.64 | 1.02E−02 |
| 107767 | Scamp1 | secretory carrier membrane protein 1 | 0.32 | 1.03E−02 |
| 18442 | P2ry2 | purinergic receptor P2Y, G-protein coupled 2 | 0.46 | 1.03E−02 |
| 214742 | Rcor3 | REST corepressor 3 | 0.57 | 1.03E−02 |
| 244882 | Tnfaip8l3 | tumor necrosis factor, alpha-induced protein 8-like | 1.34 | 1.09E−02 |
| | | 3 | | |
| 234878 | Map3k21 | mitogen-activated protein kinase kinase kinase 21 | 0.61 | 1.09E−02 |
| 14825 | Cxcl1 | chemokine (C-X-C motif) ligand 1 | 0.80 | 1.10E−02 |
| 16596 | Klf1 | Kruppel-like factor 1 (erythroid) | 0.68 | 1.11E−02 |
| 68184 | Denr | density-regulated protein | 0.34 | 1.11E−02 |
| 66090 | Ypel3 | yippee like 3 | 0.43 | 1.13E−02 |
| 232146 | Eva1a | eva-1 homolog A (C. elegans) | 0.33 | 1.13E−02 |
| 19662 | Rbp4 | retinol binding protein 4, plasma | 0.41 | 1.14E−02 |
| 230594 | Tut4 | terminal uridylyl transferase 4 | 0.41 | 1.15E−02 |
| 20219 | Apcs | amyloid P component, serum | 0.37 | 1.15E−02 |
| 21749 | Terf1 | telomeric repeat binding factor 1 | 0.72 | 1.15E−02 |
| 19201 | Pstpip2 | proline-serine-threonine phosphatase-interacting | 0.53 | 1.21E−02 |
| | | protein 2 | | |
| 50905 | Il17rb | interleukin 17 receptor B | 0.75 | 1.24E−02 |
| 71982 | Snx10 | sorting nexin 10 | 0.48 | 1.24E−02 |
| 56535 | Pex3 | peroxisomal biogenesis factor 3 | 0.34 | 1.25E−02 |
| 19157 | Cyth1 | cytohesin 1 | 0.49 | 1.25E−02 |
| 71371 | Arid5b | AT rich interactive domain 5B (MRF1-like) | 0.64 | 1.26E−02 |
| 384763 | Zfp667 | zinc finger protein 667 | 0.76 | 1.31E−02 |
| 55994 | Smad9 | SMAD family member 9 | 0.90 | 1.33E−02 |
| 232334 | Vgll4 | vestigial like family member 4 | 0.45 | 1.36E−02 |
| 80707 | Wwox | WW domain-containing oxidoreductase | 0.56 | 1.36E−02 |
| 235459 | Gtf2a2 | general transcription factor II A, 2 | 0.51 | 1.36E−02 |
| 57373 | Akip 1 | A kinase (PRKA) interacting protein 1 | 0.40 | 1.36E−02 |
| 241494 | Zfp385b | zinc finger protein 385B | 0.51 | 1.37E−02 |
| 14388 | Gab1 | growth factor receptor bound protein 2-associated | 0.47 | 1.37E−02 |
| | | protein 1 | | |
| 17135 | Mafk | v-maf musculoaponeurotic fibrosarcoma oncogene | 0.49 | 1.37E−02 |
| | | family, protein K (avian) | | |
| 26356 | Ing1 | inhibitor of growth family, member 1 | 0.47 | 1.38E−02 |
| 18643 | Pfn1 | profilin 1 | 0.26 | 1.38E−02 |
| 71091 | Cdkl1 | cyclin-dependent kinase-like 1 (CDC2-related | 1.37 | 1.38E−02 |
| | | kinase) | | |
| 19414 | Rasa3 | RAS p21 protein activator 3 | 0.48 | 1.40E−02 |
| 66853 | Pnpla2 | patatin-like phospholipase domain containing 2 | 0.57 | 1.40E−02 |
| 17134 | Mafg | v-maf musculoaponeurotic fibrosarcoma oncogene | | |
| | | family, protein G (avian) | 0.41 | 1.41E−02 |
| 211770 | Trib1 | tribbles pseudokinase 1 | 0.54 | 1.52E−02 |
| 110521 | Hivep1 | human immunodeficiency virus type I enhancer | 0.43 | 1.59E−02 |
| | | binding protein 1 | | |
| 63828 | Fn3k | fructosamine 3 kinase | 0.52 | 1.67E−02 |
| 18557 | Cdk18 | cyclin-dependent kinase 18 | 0.47 | 1.71E−02 |
| 64898 | Lpin2 | lipin 2 | 0.54 | 1.71E−02 |
| 67332 | Snrpd3 | small nuclear ribonucleoprotein D3 | 0.57 | 1.73E−02 |
| 67492 | Zfand4 | zinc finger, AN1-type domain 4 | 0.44 | 1.78E−02 |
| 97130 | C77080 | expressed sequence C77080 | 0.45 | 1.80E−02 |
| 100503969 | 1810008l18Rik | RIKEN cDNA 1810008118 gene | 0.37 | 1.80E−02 |
| 108112 | Eif4ebp3 | eukaryotic translation initiation factor 4E binding | | |
| | | protein 3 | 0.74 | 1.83E−02 |
| 268417 | Zkscan17 | zinc finger with KRAB and SCAN domains 17 | 0.49 | 1.83E−02 |
| 192656 | Ripk2 | receptor (TNFRSF)-interacting serine-threonine | 0.50 | 1.92E−02 |
| | | kinase 2 | | |
| 17771 | Tesmin | testis expressed metallothionein like | 1.08 | 1.95E−02 |
| 217039 | Ggnbp2 | gametogenetin binding protein 2 | 0.28 | 1.96E−02 |
| 69786 | Tprkb | Tp53rk binding protein | 0.35 | 1.96E−02 |
| 320332 | H4f16 | H4 histone 16 | 0.59 | 1.96E−02 |
| 69090 | Ascc1 | activating signal cointegrator 1 complex subunit 1 | 0.37 | 2.03E−02 |
| 384071 | Slc25a34 | solute carrier family 25, member 34 | 0.77 | 2.03E−02 |
| 15357 | Hmgcr | 3-hydroxy-3-methylglutaryl-Coenzyme A reductase | 0.60 | 2.04E−02 |
| 329977 | Fhad1 | forkhead-associated (FHA) phosphopeptide | 0.82 | 2.07E−02 |
| | | binding domain 1 | | |
| 11539 | Adora1 | adenosine A1 receptor | 0.68 | 2.08E−02 |
| 17119 | Mxd1 | MAX dimerization protein 1 | 0.52 | 2.09E−02 |
| 13361 | Dhfr | dihydrofolate reductase | 0.29 | 2.10E−02 |
| 22032 | Traf4 | TNF receptor associated factor 4 | 0.36 | 2.11E−02 |
| 28075 | Desi1 | desumoylating isopeptidase 1 | 0.38 | 2.14E−02 |
| 11459 | Acta1 | actin, alpha 1, skeletal muscle | 5.28 | 2.14E−02 |
| 214804 | Syde2 | synapse defective 1, Rho GTPase, homolog 2 (C. | 0.63 | 2.16E−02 |
| | | elegans) | | |
| 58520 | Erg28 | ergosterol biosynthesis 28 | 0.43 | 2.16E−02 |
| 12122 | Bid | BH3 interacting domain death agonist | 0.42 | 2.16E−02 |
| 68520 | Zfyve21 | zinc finger, FYVE domain containing 21 | 0.43 | 2.25E−02 |
| 19401 | Rara | retinoic acid receptor, alpha | 0.53 | 2.28E−02 |
| 64540 | Tspan4 | tetraspanin 4 | 0.33 | 2.30E−02 |
| 13853 | Epm2a | epilepsy, progressive myoclonic epilepsy, type 2 | 0.38 | 2.45E−02 |
| | | gene alpha | | |
| 66248 | Alg5 | asparagine-linked glycosylation 5 (dolichyl- | 0.39 | 2.45E−02 |
| | | phosphate beta-glucosyltransferase) | | |
| 57441 | Gmnn | geminin | 0.65 | 2.45E−02 |
| 17872 | Ppp1r15a | protein phosphatase 1, regulatory subunit 15A | 0.54 | 2.45E−02 |
| 19041 | Ppl | periplakin | 0.76 | 2.56E−02 |
| 27966 | Rrp9 | ribosomal RNA processing 9, U3 small nucleolar | 0.46 | 2.56E−02 |
| | | RNA binding protein | | |
| 214137 | Arhgap29 | Rho GTPase activating protein 29 | 0.36 | 2.56E−02 |
| 21816 | Tgm1 | transglutaminase 1, K polypeptide | 0.77 | 2.63E−02 |
| 19376 | Rab34 | RAB34, member RAS oncogene family | 0.70 | 2.63E−02 |
| 22262 | Uox | urate oxidase | 0.33 | 2.63E−02 |
| 12580 | Cdkn2c | cyclin dependent kinase inhibitor 2C | 0.65 | 2.65E−02 |
| 52432 | Ppp2r2d | protein phosphatase 2, regulatory subunit B, delta | 0.32 | 2.76E−02 |
| 12953 | Cry2 | cryptochrome 2 (photolyase-like) | 0.47 | 2.83E−02 |
| 227298 | Retreg2 | reticulophagy regulator family member 2 | 0.24 | 2.86E−02 |
| 56613 | Rps6ka4 | ribosomal protein S6 kinase, polypeptide 4 | 0.50 | 2.89E−02 |
| 15512 | Hspa2 | heat shock protein 2 | 0.77 | 2.90E−02 |
| 20410 | Sorbs3 | sorbin and SH3 domain containing 3 | 0.59 | 2.93E−02 |
| 21877 | Tk1 | thymidine kinase 1 | 0.52 | 2.97E−02 |
| 242669 | Azin2 | antizyme inhibitor 2 | 1.11 | 2.97E−02 |
| 231070 | Insig1 | insulin induced gene 1 | 0.44 | 3.03E−02 |
| 15903 | Id3 | inhibitor of DNA binding 3 | 0.58 | 3.03E−02 |
| 74360 | Cep57 | centrosomal protein 57 | 0.46 | 3.12E−02 |
| 71994 | Cnn3 | calponin 3, acidic | 0.31 | 3.12E−02 |
| 216395 | Rxylt1 | ribitol xylosyltransferase 1 | 0.44 | 3.12E−02 |
| 69171 | Cnppd1 | cyclin Pas1/PHO80 domain containing 1 | 0.35 | 3.12E−02 |
| 16476 | Jun | jun proto-oncogene | 0.67 | 3.12E−02 |
| 67800 | Dgat2 | diacylglycerol O-acyltransferase 2 | 0.25 | 3.14E−02 |
| 12527 | Cd9 | CD9 antigen | 0.54 | 3.19E−02 |
| 13197 | Gadd45a | growth arrest and DNA-damage-inducible 45 alpha | 0.91 | 3.20E−02 |
| 108645 | Mat2b | methionine adenosyltransferase II, beta | 0.33 | 3.21E−02 |
| 15284 | Hlx | H2.0-like homeobox | 0.78 | 3.21E−02 |
| 22004 | Tpm2 | tropomyosin 2, beta | 0.75 | 3.23E−02 |
| 56441 | Naa80 | N(alpha)-acetyltransferase 80, NatH catalytic | 0.39 | 3.27E−02 |
| | | subunit | | |
| 67477 | Abhd15 | abhydrolase domain containing 15 | 0.39 | 3.30E−02 |
| 78232 | Trappc6b | trafficking protein particle complex 6B | 0.40 | 3.32E−02 |
| 106628 | Trip10 | thyroid hormone receptor interactor 10 | 0.47 | 3.37E−02 |
| 57339 | Jph1 | junctophilin 1 | 1.18 | 3.37E−02 |
| 70556 | Slc25a33 | solute carrier family 25, member 33 | 0.44 | 3.38E−02 |
| 384198 | Fam47e | family with sequence similarity 47, member E | 0.69 | 3.41E−02 |
| 12927 | Bcar1 | breast cancer anti-estrogen resistance 1 | 0.39 | 3.47E−02 |
| 20768 | Sephs2 | selenophosphate synthetase 2 | 0.34 | 3.51E−02 |
| 23830 | Capn10 | calpain 10 | 0.39 | 3.56E−02 |
| 13716 | EII | elongation factor RNA polymerase II | 0.38 | 3.62E−02 |
| 14251 | Flot1 | flotillin 1 | 0.40 | 3.65E−02 |
| 27370 | Rps26 | ribosomal protein S26 | 0.38 | 3.76E−02 |
| 12408 | Cbr1 | carbonyl reductase 1 | 0.34 | 3.76E−02 |
| 58800 | Trpm7 | transient receptor potential cation channel, | 0.22 | 3.82E−02 |
| | | subfamily M, member 7 | | |
| 14957 | H1f3 | H1.3 linker histone, cluster member | 0.46 | 3.86E−02 |
| 11992 | Auh | AU RNA binding protein/enoyl-coenzyme A | 0.41 | 3.87E−02 |
| | | hydratase | | |
| 27400 | Hsd17b6 | hydroxysteroid (17-beta) dehydrogenase 6 | 0.39 | 3.93E−02 |
| 16206 | Lrig1 | leucine-rich repeats and immunoglobulin-like | 0.31 | 3.93E−02 |
| | | domains 1 | | |
| 227449 | Zcchc2 | zinc finger, CCHC domain containing 2 | 0.33 | 3.96E−02 |
| 27399 | Ip6k1 | inositol hexaphosphate kinase 1 | 0.24 | 3.96E−02 |
| 94284 | Ugt1a6a | UDP glucuronosyltransferase 1 family, polypeptide | 0.91 | 4.01E−02 |
| | | A6A | | |
| 238330 | Irf2bpl | interferon regulatory factor 2 binding protein-like | 0.41 | 4.05E−02 |
| 22217 | Usp12 | ubiquitin specific peptidase 12 | 0.29 | 4.05E−02 |
| 18637 | Pfdn2 | prefoldin 2 | 0.35 | 4.06E−02 |
| 212073 | Syne3 | spectrin repeat containing, nuclear envelope family | 0.57 | 4.08E−02 |
| | | member 3 | | |
| 103537 | Mbtd1 | mbt domain containing 1 | 0.48 | 4.10E−02 |
| 22210 | Ube2b | ubiquitin-conjugating enzyme E2B | 0.41 | 4.14E−02 |
| 15199 | Hebp1 | heme binding protein 1 | 0.38 | 4.14E−02 |
| 68146 | Arl13b | ADP-ribosylation factor-like 13B | 0.37 | 4.26E−02 |
| 240023 | Pnldc1 | poly(A)-specific ribonuclease (PARN)-like domain | 0.83 | 4.26E−02 |
| | | containing 1 | | |
| 103724 | Tbc1d10a | TBC1 domain family, member 10a | 0.61 | 4.30E−02 |
| 449000 | Zfp960 | zinc finger protein 960 | 0.60 | 4.32E−02 |
| 114584 | Clic1 | chloride intracellular channel 1 | 0.35 | 4.41E−02 |
| 20054 | Rps15 | ribosomal protein S15 | 0.38 | 4.47E−02 |
| 100503178 | 2810013P06Rik | RIKEN cDNA 2810013P06 gene | 0.57 | 4.48E−02 |
| 330938 | Dixdc1 | DIX domain containing 1 | 0.32 | 4.52E−02 |
| 67443 | Map1lc3b | microtubule-associated protein 1 light chain 3 beta | 0.28 | 4.64E−02 |
| 18569 | Pdcd4 | programmed cell death 4 | 0.36 | 4.65E−02 |
| 331535 | Serpina7 | serine (or cysteine) peptidase inhibitor, clade A | 0.41 | 4.65E−02 |
| | | (alpha-1 antiproteinase, antitrypsin), member 7 | | |
| 22722 | Zfp64 | zinc finger protein 64 | 0.41 | 4.72E−02 |
| 16147 | Ihh | Indian hedgehog | 0.74 | 4.72E−02 |
| 59028 | Rcl1 | RNA terminal phosphate cyclase-like 1 | 0.28 | 4.73E−02 |
| 71661 | 0610005C13Rik | RIKEN cDNA 0610005C13 gene | 0.39 | 4.75E−02 |
| | | SWI/SNF related, matrix associated, actin | | |
| 83796 | Smarcd2 | dependent regulator of chromatin, subfamily d, | | |
| | | member 2 | 0.27 | 4.75E−02 |
| 108099 | Prkag2 | protein kinase, AMP-activated, gamma 2 non- | 0.36 | 4.76E−02 |
| | | catalytic subunit | | |
| 12448 | Ccne2 | cyclin E2 | 0.70 | 4.78E−02 |
| 72016 | Tedc2 | tubulin epsilon and delta complex 2 | 0.43 | 4.80E−02 |
| 56207 | Uchl5 | ubiquitin carboxyl-terminal esterase L5 | 0.34 | 4.83E−02 |
| 108160 | Fam50a | family with sequence similarity 50, member A | 0.41 | 4.83E−02 |
| 70266 | Kyat1 | kynurenine aminotransferase 1 | 0.24 | 4.88E−02 |
| 17859 | Mxi1 | MAX interactor 1, dimerization protein | 0.29 | 4.88E−02 |
| 67647 | 4930523C07Rik | RIKEN cDNA 4930523C07 gene | 0.33 | 4.89E−02 |
| 213056 | Fam126b | family with sequence similarity 126, member B | 0.31 | 4.91E−02 |
| 78751 | Zc3h6 | zinc finger CCCH type containing 6 | 0.63 | 4.95E−02 |
| 77128 | Crebrf | CREB3 regulatory factor | 0.32 | 4.99E−02 |

TABLE S3B
Downregulated genes in liver.

| | | | | FDR- |
|---|---|---|---|---|
| | | | | adjusted |
| | | | | Wald |
| Entrez | | | log2 fold- | test P- |
| Gene ID | Symbol | Name | change | value |
| 12686 | Elovl3 | elongation of very long chain fatty acids (FEN1/Elo2, | −2.15 | 4.65E−61 |
| | | SUR4/Elo3, yeast)-like 3 | | |
| 22151 | Tubb2a | tubulin, beta 2A class IIA | −2.10 | 1.50E−50 |
| 72999 | Insig2 | insulin induced gene 2 | −1.41 | 8.03E−36 |
| 14773 | Grk5 | G protein-coupled receptor kinase 5 | −2.35 | 6.83E−32 |
| 11865 | Arntl | aryl hydrocarbon receptor nuclear translocator-like | −2.61 | 9.45E−25 |
| 12660 | Chka | choline kinase alpha | −2.04 | 1.88E−23 |
| 226781 | Slc30a10 | solute carrier family 30, member 10 | −1.41 | 3.33E−23 |
| 270166 | Clpx | caseinolytic mitochondrial matrix peptidase chaperone subunit | −1.29 | 1.28E−17 |
| 12753 | Clock | circadian locomotor output cycles kaput | −0.78 | 3.38E−17 |
| 99382 | Abtb2 | ankyrin repeat and BTB (POZ) domain containing 2 | −1.37 | 5.56E−15 |
| 21807 | Tsc22d1 | TSC22 domain family, member 1 | −1.70 | 1.45E−14 |
| 241452 | Dhrs9 | dehydrogenase/reductase (SDR family) member 9 | −2.12 | 1.87E−14 |
| 207521 | Dtx4 | deltex 4, E3 ubiquitin ligase | −1.30 | 1.89E−14 |
| 212980 | Slc45a3 | solute carrier family 45, member 3 | −1.18 | 1.89E−14 |
| 12737 | Cldn1 | claudin 1 | −1.03 | 2.29E−13 |
| 15519 | Hsp90aa1 | heat shock protein 90, alpha (cytosolic), class A member 1 | −0.81 | 3.02E−13 |
| 13195 | Ddc | dopa decarboxylase | −1.08 | 1.61E−12 |
| 77864 | Ypel2 | yippee like 2 | −1.36 | 5.43E−12 |
| 53872 | Caprin1 | cell cycle associated protein 1 | −0.65 | 7.73E−12 |
| 55963 | Slc1a4 | solute carrier family 1 (glutamate/neutral amino acid | −1.67 | 8.26E−12 |
| | | transporter), member 4 | | |
| 67426 | Coq8a | coenzyme Q8A | −0.89 | 8.59E−12 |
| 13124 | Cyp8b1 | cytochrome P450, family 8, subfamily b, polypeptide 1 | −0.91 | 2.12E−11 |
| 71751 | Map3k13 | mitogen-activated protein kinase kinase kinase 13 | −1.48 | 3.47E−11 |
| 235497 | Leo1 | Leo1, Paf1/RNA polymerase II complex component | −1.07 | 3.55E−11 |
| 216551 | Lgalsl | lectin, galactoside binding-like | −1.05 | 1.26E−10 |
| 14828 | Hspa5 | heat shock protein 5 | −0.64 | 3.05E−10 |
| 72480 | Tspyl4 | TSPY-like 4 | −1.90 | 7.28E−10 |
| 233067 | Lrfn3 | leucine rich repeat and fibronectin type III domain containing 3 | −1.76 | 1.19E−09 |
| 27273 | Pdk4 | pyruvate dehydrogenase kinase, isoenzyme 4 | −1.49 | 1.93E−09 |
| 56349 | Net1 | neuroepithelial cell transforming gene 1 | −0.96 | 2.38E−09 |
| 19165 | Psen2 | presenilin 2 | −0.54 | 4.74E−09 |
| 18030 | Nfil3 | nuclear factor, interleukin 3, regulated | −1.16 | 7.09E−09 |
| 78825 | Desi2 | desumoylating isopeptidase 2 | −0.88 | 7.99E−09 |
| 17769 | Mthfr | methylenetetrahydrofolate reductase | −1.28 | 2.30E−08 |
| 233752 | Insc | INSC spindle orientation adaptor protein | −1.09 | 3.36E−08 |
| 227545 | Proser2 | proline and serine rich 2 | −0.97 | 5.13E−08 |
| 66917 | Chordc1 | cysteine and histidine-rich domain (CHORD)-containing, zinc- | −0.61 | 6.01E−08 |
| | | binding protein 1 | | |
| 20442 | St3gal1 | ST3 beta-galactoside alpha-2,3-sialyltransferase 1 | −0.76 | 1.22E−07 |
| 211914 | Asap2 | ArfGAP with SH3 domain, ankyrin repeat and PH domain 2 | −1.20 | 1.65E−07 |
| 67040 | Ddx17 | DEAD box helicase 17 | −0.83 | 2.04E−07 |
| 66612 | Ormdl3 | ORM1-like 3 (S. cerevisiae) | −0.57 | 2.56E−07 |
| 74197 | Gtf2e1 | general transcription factor II E, polypeptide 1 (alpha subunit) | −0.83 | 2.92E−07 |
| 20850 | Stat5a | signal transducer and activator of transcription 5A | −1.10 | 2.99E−07 |
| 18451 | P4ha1 | procollagen-proline, 2-oxoglutarate 4-dioxygenase (proline 4- | −1.06 | 3.39E−07 |
| | | hydroxylase), alpha 1 polypeptide | | |
| 67996 | Srsf6 | serine and arginine-rich splicing factor 6 | −0.58 | 4.41E−07 |
| 75552 | Paqr9 | progestin and adipoQ receptor family member IX | −0.70 | 5.11E−07 |
| 381269 | Mreg | melanoregulin | −0.70 | 6.31E−07 |
| 20482 | Skil | SKI-like | −0.60 | 7.38E−07 |
| 100689 | Spon2 | spondin 2, extracellular matrix protein | −1.08 | 1.41E−06 |
| 18742 | Pitx3 | paired-like homeodomain transcription factor 3 | −2.98 | 1.41E−06 |
| 71704 | Arhgef3 | Rho guanine nucleotide exchange factor (GEF) 3 | −0.58 | 1.75E−06 |
| 70984 | 4931406C07Rik | RIKEN cDNA 4931406C07 gene | −0.67 | 2.30E−06 |
| 15505 | Hsph1 | heat shock 105 kDa/110 kDa protein 1 | −1.07 | 2.30E−06 |
| 58185 | Rsad2 | radical S-adenosyl methionine domain containing 2 | −0.85 | 2.59E−06 |
| 67664 | Rnf125 | ring finger protein 125 | −0.70 | 3.29E−06 |
| 22190 | Ubc | ubiquitin C | −0.65 | 3.58E−06 |
| 218215 | Rnf144b | ring finger protein 144B | −0.57 | 3.96E−06 |
| 11520 | Plin2 | perilipin 2 | −0.62 | 5.09E−06 |
| 12952 | Cry1 | cryptochrome 1 (photolyase-like) | −1.28 | 5.59E−06 |
| 68192 | Leprotl1 | leptin receptor overlapping transcript-like 1 | −0.54 | 7.17E−06 |
| 13609 | S1pr1 | sphingosine-1-phosphate receptor 1 | −0.72 | 7.70E−06 |
| 69635 | Dapk1 | death associated protein kinase 1 | −0.75 | 9.89E−06 |
| 22042 | Tfrc | transferrin receptor | −0.85 | 9.91E−06 |
| 20531 | Slc34a2 | solute carrier family 34 (sodium phosphate), member 2 | −4.30 | 1.05E−05 |
| 20501 | Slc16a1 | solute carrier family 16 (monocarboxylic acid transporters), | −0.72 | 1.44E−05 |
| | | member 1 | | |
| 69627 | Fam89a | family with sequence similarity 89, member A | −1.57 | 1.45E−05 |
| 108767 | Pnrc1 | proline-rich nuclear receptor coactivator 1 | −0.67 | 1.75E−05 |
| 110960 | Tars | threonyl-tRNA synthetase | −0.80 | 1.82E−05 |
| 12116 | Bhmt | betaine-homocysteine methyltransferase | −0.48 | 1.91E−05 |
| 338365 | Slc41a2 | solute carrier family 41, member 2 | −0.88 | 2.10E−05 |
| 12406 | Serpinh1 | serine (or cysteine) peptidase inhibitor, clade H, member 1 | −0.70 | 2.24E−05 |
| 213417 | Klhdc8a | kelch domain containing 8A | −1.56 | 2.26E−05 |
| 231630 | Ficd | FIC domain containing | −0.70 | 3.17E−05 |
| 16402 | Itga5 | integrin alpha 5 (fibronectin receptor alpha) | −0.93 | 3.54E−05 |
| 98845 | Eps8l2 | EPS8-like 2 | −0.64 | 3.93E−05 |
| 74114 | Crot | carnitine O-octanoyltransferase | −0.51 | 4.06E−05 |
| 328133 | Slc39a9 | solute carrier family 39 (zinc transporter), member 9 | −0.49 | 4.72E−05 |
| 17773 | Mtnr1a | melatonin receptor 1A | −1.12 | 5.23E−05 |
| 56844 | Tssc4 | tumor-suppressing subchromosomal transferable fragment 4 | −0.77 | 5.96E−05 |
| 83814 | Nedd4l | neural precursor cell expressed, developmentally down- | | |
| | | regulated gene 4-like | −0.76 | 1.18E−04 |
| 59038 | Pxmp4 | peroxisomal membrane protein 4 | −0.58 | 1.22E−04 |
| 12053 | Bcl6 | B cell leukemia/lymphoma 6 | −2.41 | 1.27E−04 |
| 76954 | Denn2b | DENN domain containing 2B | −0.86 | 1.27E−04 |
| 18174 | Slc11a2 | solute carrier family 11 (proton-coupled divalent metal ion | −0.56 | 1.27E−04 |
| | | transporters), member 2 | | |
| 54140 | Avpr1a | arginine vasopressin receptor 1A | −0.65 | 1.32E−04 |
| 72333 | Palld | palladin, cytoskeletal associated protein | −0.81 | 1.72E−04 |
| 230161 | Acnat1 | acyl-coenzyme A amino acid N-acyltransferase 1 | −0.48 | 1.84E−04 |
| 29815 | Bcar3 | breast cancer anti-estrogen resistance 3 | −0.57 | 1.93E−04 |
| 213649 | Arhgef19 | Rho guanine nucleotide exchange factor (GEF) 19 | −0.70 | 1.97E−04 |
| 207259 | Zbtb7c | zinc finger and BTB domain containing 7C | −2.15 | 1.99E−04 |
| 12614 | Celsr1 | cadherin, EGF LAG seven-pass G-type receptor 1 | −1.00 | 2.03E−04 |
| 22169 | Cmpk2 | cytidine monophosphate (UMP-CMP) kinase 2, mitochondrial | −0.77 | 2.19E−04 |
| 211948 | Pde12 | phosphodiesterase 12 | −0.59 | 2.27E−04 |
| 108100 | Baiap2 | brain-specific angiogenesis inhibitor 1-associated protein 2 | −0.59 | 2.29E−04 |
| 67573 | Loxl4 | lysyl oxidase-like 4 | −1.61 | 2.47E−04 |
| 225358 | Fam13b | family with sequence similarity 13, member B | −0.57 | 2.60E−04 |
| 12944 | Crp | C-reactive protein, pentraxin-related | −0.32 | 2.70E−04 |
| 227613 | Tubb4b | tubulin, beta 4B class IVB | −0.57 | 2.71E−04 |
| 14287 | Fpgs | folylpolyglutamyl synthetase | −0.41 | 3.07E−04 |
| 18950 | Pnp | purine-nucleoside phosphorylase | −0.51 | 3.07E−04 |
| 15953 | Ifi47 | interferon gamma inducible protein 47 | −0.63 | 3.34E−04 |
| 320878 | Mical2 | microtubule associated monooxygenase, calponin and LIM | −0.96 | 3.73E−04 |
| | | domain containing 2 | | |
| 226747 | Ahctf1 | AT hook containing transcription factor 1 | −0.57 | 3.87E−04 |
| 53619 | Blcap | bladder cancer associated protein | −0.52 | 4.30E−04 |
| 56632 | Sphk2 | sphingosine kinase 2 | −0.49 | 4.38E−04 |
| 73699 | Ppp2r1b | protein phosphatase 2, regulatory subunit A, beta | −0.48 | 4.56E−04 |
| 78903 | Wrnip1 | Werner helicase interacting protein 1 | −0.58 | 5.27E−04 |
| 67846 | Tmem39a | transmembrane protein 39a | −0.60 | 5.69E−04 |
| 109154 | Mlec | malectin | −0.41 | 5.82E−04 |
| 70789 | Kynu | kynureninase | −0.35 | 6.58E−04 |
| 68652 | Tab2 | TGF-beta activated kinase 1/MAP3K7 binding protein 2 | −0.43 | 8.26E−04 |
| 71890 | Mad2l2 | MAD2 mitotic arrest deficient-like 2 | −0.54 | 8.33E−04 |
| 140887 | Lnx2 | ligand of numb-protein X 2 | −0.86 | 8.91E−04 |
| 229227 | 4932438A13Rik | RIKEN cDNA 4932438A13 gene | −0.58 | 1.12E−03 |
| 68839 | Ankrd46 | ankyrin repeat domain 46 | −0.33 | 1.14E−03 |
| 319582 | Trmt9b | tRNA methyltransferase 9B | −0.78 | 1.15E−03 |
| 270110 | Irf2bp2 | interferon regulatory factor 2 binding protein 2 | −0.71 | 1.20E−03 |
| 18585 | Pde9a | phosphodiesterase 9A | −0.52 | 1.21E−03 |
| 14827 | Pdia3 | protein disulfide isomerase associated 3 | −0.31 | 1.23E−03 |
| 215748 | Cnksr3 | Cnksr family member 3 | −0.72 | 1.36E−03 |
| 23989 | Med24 | mediator complex subunit 24 | −0.55 | 1.42E−03 |
| 55989 | Nop58 | NOP58 ribonucleoprotein | −0.46 | 1.43E−03 |
| 71137 | Rfx4 | regulatory factor X, 4 (influences HLA class II expression) | −0.72 | 1.43E−03 |
| 68337 | Crip2 | cysteine rich protein 2 | −0.51 | 1.46E−03 |
| 23881 | G3bp2 | GTPase activating protein (SH3 domain) binding protein 2 | −0.36 | 1.47E−03 |
| 20815 | Srpk1 | serine/arginine-rich protein specific kinase 1 | −0.46 | 1.54E−03 |
| 15944 | Irgm1 | immunity-related GTPase family M member 1 | −0.48 | 1.62E−03 |
| 74018 | Als2 | alsin Rho guanine nucleotide exchange factor | −0.48 | 1.72E−03 |
| 104859 | Tecpr2 | tectonin beta-propeller repeat containing 2 | −0.54 | 1.72E−03 |
| 71724 | Aox3 | aldehyde oxidase 3 | −0.50 | 1.74E−03 |
| 22146 | Tuba1c | tubulin, alpha 1C | −0.57 | 2.19E−03 |
| 16518 | Kcnj2 | potassium inwardly-rectifying channel, subfamily J, member 2 | −1.65 | 2.22E−03 |
| 104681 | Slc16a6 | solute carrier family 16 (monocarboxylic acid transporters), | −1.40 | 2.46E−03 |
| | | member 6 | | |
| 240549 | Gm4952 | predicted gene 4952 | −0.35 | 2.46E−03 |
| 56722 | Litaf | LPS-induced TN factor | −0.38 | 2.62E−03 |
| 67812 | Ubxn4 | UBX domain protein 4 | −0.32 | 2.74E−03 |
| 104174 | Gldc | glycine decarboxylase | −0.62 | 3.00E−03 |
| 224897 | Dpp9 | dipeptidylpeptidase 9 | −0.41 | 3.08E−03 |
| 18226 | Nup62 | nucleoporin 62 | −0.71 | 3.28E−03 |
| 28042 | Selenoi | selenoprotein I | −0.55 | 3.33E−03 |
| 74152 | Stra6l | STRA6-like | −0.51 | 3.40E−03 |
| 27413 | Abcb11 | ATP-binding cassette, sub-family B (MDR/TAP), member 11 | −0.43 | 3.40E−03 |
| 16325 | Inhbc | inhibin beta-C | −0.33 | 3.64E−03 |
| 80890 | Trim2 | tripartite motif-containing 2 | −0.48 | 3.82E−03 |
| 12978 | Csf1r | colony stimulating factor 1 receptor | −0.52 | 3.86E−03 |
| 210146 | Irgq | immunity-related GTPase family, Q | −0.57 | 3.90E−03 |
| 18143 | Npas2 | neuronal PAS domain protein 2 | −1.54 | 3.93E−03 |
| 223881 | Rnd1 | Rho family GTPase 1 | −0.70 | 3.96E−03 |
| 30935 | Tor3a | torsin family 3, member A | −0.80 | 4.00E−03 |
| 381823 | Apold1 | apolipoprotein L domain containing 1 | −0.96 | 4.35E−03 |
| 13664 | Eif1a | eukaryotic translation initiation factor 1A | −0.48 | 4.35E−03 |
| 72338 | Wdr89 | WD repeat domain 89 | −0.96 | 4.41E−03 |
| 67434 | Ankrd33b | ankyrin repeat domain 33B | −0.43 | 4.58E−03 |
| 21928 | Tnfaip2 | tumor necrosis factor, alpha-induced protein 2 | −0.77 | 4.79E−03 |
| 18670 | Abcb4 | ATP-binding cassette, sub-family B (MDR/TAP), member 4 | −0.40 | 5.06E−03 |
| 110816 | Pwp2 | PWP2 periodic tryptophan protein homolog (yeast) | −0.47 | 5.07E−03 |
| 50770 | Atp11a | ATPase, class VI, type 11A | −0.61 | 5.12E−03 |
| 320806 | Gfm2 | G elongation factor, mitochondrial 2 | −0.30 | 5.27E−03 |
| 12915 | Atf6b | activating transcription factor 6 beta | −0.51 | 5.28E−03 |
| 218333 | Ice1 | interactor of little elongation complex ELL subunit 1 | −0.43 | 5.41E−03 |
| 12274 | C6 | complement component 6 | −0.45 | 5.42E−03 |
| 12916 | Crem | cAMP responsive element modulator | −0.50 | 5.52E−03 |
| 244416 | Ppp1r3b | protein phosphatase 1, regulatory subunit 3B | −1.09 | 5.72E−03 |
| 15525 | Hspa4 | heat shock protein 4 | −0.35 | 6.47E−03 |
| 72727 | B3gat3 | beta-1,3-glucuronyltransferase 3 (glucuronosyltransferase I) | −0.38 | 6.50E−03 |
| 232748 | Tcaf2 | TRPM8 channel-associated factor 2 | −1.15 | 6.55E−03 |
| 104662 | Tsr1 | TSR1 20S rRNA accumulation | −0.51 | 6.55E−03 |
| 230676 | Szt2 | SZT2 subunit of KICSTOR complex | −0.61 | 6.99E−03 |
| 20677 | Sox4 | SRY (sex determining region Y)-box 4 | −0.70 | 7.43E−03 |
| 69674 | Mif4gd | MIF4G domain containing | −0.36 | 7.65E−03 |
| 11491 | Adam17 | a disintegrin and metallopeptidase domain 17 | −0.44 | 7.81E−03 |
| 241633 | Atp8b4 | ATPase, class I, type 8B, member 4 | −0.76 | 7.98E−03 |
| 71093 | Atoh8 | atonal bHLH transcription factor 8 | −0.88 | 8.01E−03 |
| 207952 | Klhl25 | kelch-like 25 | −0.62 | 8.18E−03 |
| 218629 | Dhx29 | DEAH (Asp-Glu-Ala-His) box polypeptide 29 | −0.46 | 8.24E−03 |
| 16367 | Irs1 | insulin receptor substrate 1 | −0.60 | 8.24E−03 |
| 53881 | Slc5a3 | solute carrier family 5 (inositol transporters), member 3 | −0.63 | 8.31E−03 |
| 241627 | Wdr76 | WD repeat domain 76 | −0.82 | 8.33E−03 |
| 231532 | Arhgap24 | Rho GTPase activating protein 24 | −0.52 | 8.77E−03 |
| 20397 | Sgpl1 | sphingosine phosphate lyase 1 | −0.44 | 8.90E−03 |
| 13733 | Adgre1 | adhesion G protein-coupled receptor E1 | −0.55 | 8.91E−03 |
| 231207 | Cpeb2 | cytoplasmic polyadenylation element binding protein 2 | −0.66 | 8.97E−03 |
| 12212 | Chic1 | cysteine-rich hydrophobic domain 1 | −0.46 | 8.97E−03 |
| 72007 | Fndc3b | fibronectin type III domain containing 3B | −0.53 | 9.05E−03 |
| 66968 | Plin5 | perilipin 5 | −0.49 | 9.20E−03 |
| 27041 | G3bp1 | GTPase activating protein (SH3 domain) binding protein 1 | −0.33 | 9.63E−03 |
| 434437 | Amt | aminomethyltransferase | −0.29 | 9.63E−03 |
| 268390 | Ahsa2 | AHA1, activator of heat shock protein ATPase 2 | −0.39 | 1.00E−02 |
| 71946 | Endod1 | endonuclease domain containing 1 | −0.70 | 1.01E−02 |
| 73173 | Pcdh18 | protocadherin 18 | −0.78 | 1.02E−02 |
| 54139 | Irf6 | interferon regulatory factor 6 | −0.31 | 1.03E−02 |
| 16796 | Lasp1 | LIM and SH3 protein 1 | −0.45 | 1.04E−02 |
| 67724 | Pop1 | processing of precursor 1, ribonuclease P/MRP family, (S. | −0.60 | 1.11E−02 |
| | | cerevisiae) | | |
| 243362 | Stard13 | StAR-related lipid transfer (START) domain containing 13 | −0.35 | 1.11E−02 |
| 13052 | Cxadr | coxsackie virus and adenovirus receptor | −0.32 | 1.11E−02 |
| 74032 | Sdr42e1 | short chain dehydrogenase/reductase family 42E, member 1 | −0.33 | 1.12E−02 |
| 18242 | Oat | ornithine aminotransferase | −0.34 | 1.12E−02 |
| 211652 | Wwc1 | WW, C2 and coiled-coil domain containing 1 | −0.34 | 1.12E−02 |
| 228602 | 4930402H24Rik | RIKEN cDNA 4930402H24 gene | −0.36 | 1.13E−02 |
| 100019 | Mdn1 | midasin AAA ATPase 1 | −0.51 | 1.13E−02 |
| 99887 | Tlcd4 | TLC domain containing 4 | −0.29 | 1.14E−02 |
| 329795 | Tmem67 | transmembrane protein 67 | −0.73 | 1.14E−02 |
| 22439 | Xk | X-linked Kx blood group | −0.52 | 1.14E−02 |
| 74340 | Ahcyl2 | S-adenosylhomocysteine hydrolase-like 2 | −0.53 | 1.15E−02 |
| 56695 | Pnkd | paroxysmal nonkinesiogenic dyskinesia | −0.43 | 1.16E−02 |
| 18746 | Pkm | pyruvate kinase, muscle | −0.45 | 1.21E−02 |
| 56737 | Alg2 | asparagine-linked glycosylation 2 (alpha-1,3- | −0.52 | 1.31E−02 |
| | | mannosyltransferase) | | |
| 21682 | Tec | tec protein tyrosine kinase | −0.33 | 1.34E−02 |
| 268490 | Lsm12 | LSM12 homolog | −0.32 | 1.35E−02 |
| 22213 | Ube2g2 | ubiquitin-conjugating enzyme E2G 2 | −0.40 | 1.36E−02 |
| 70885 | Ints10 | integrator complex subunit 10 | −0.49 | 1.38E−02 |
| 72479 | Hsdl2 | hydroxysteroid dehydrogenase like 2 | −0.29 | 1.38E−02 |
| 217837 | Itpk1 | inositol 1,3,4-triphosphate 5/6 kinase | −0.39 | 1.38E−02 |
| 27375 | Tjp3 | tight junction protein 3 | −0.60 | 1.38E−02 |
| 232670 | Tspan33 | tetraspanin 33 | −0.42 | 1.39E−02 |
| 71323 | Rassf8 | Ras association (RalGDS/AF-6) domain family (N-terminal) | −0.31 | 1.39E−02 |
| | | member 8 | | |
| 72962 | Tymp | thymidine phosphorylase | −0.43 | 1.39E−02 |
| 105853 | Mal2 | mal, T cell differentiation protein 2 | −0.54 | 1.40E−02 |
| 277753 | Cyp4a12a | cytochrome P450, family 4, subfamily a, polypeptide 12a | −0.58 | 1.44E−02 |
| 328580 | Tubgcp6 | tubulin, gamma complex associated protein 6 | −0.50 | 1.44E−02 |
| 22235 | Ugdh | UDP-glucose dehydrogenase | −0.23 | 1.45E−02 |
| 16889 | Lipa | lysosomal acid lipase A | −0.33 | 1.47E−02 |
| 14218 | Sh3pxd2a | SH3 and PX domains 2A | −0.42 | 1.48E−02 |
| 12350 | Car3 | carbonic anhydrase 3 | −0.36 | 1.56E−02 |
| 108960 | Irak2 | interleukin-1 receptor-associated kinase 2 | −0.42 | 1.59E−02 |
| 66978 | Luc7l | Luc7-like | −0.38 | 1.61E−02 |
| 20776 | Tmie | transmembrane inner ear | −0.46 | 1.61E−02 |
| 80748 | BC004004 | cDNA sequence BC004004 | −0.24 | 1.67E−02 |
| 18053 | Ngfr | nerve growth factor receptor (TNFR superfamily, member 16) | −0.67 | 1.67E−02 |
| 108155 | Ogt | O-linked N-acetylglucosamine (GlcNAc) transferase (UDP-N- | −0.36 | 1.70E−02 |
| | | acetylglucosamine:polypeptide-N-acetylglucosaminyl | | |
| | | transferase) | | |
| 67075 | Magt1 | magnesium transporter 1 | −0.30 | 1.71E−02 |
| 16763 | Lad1 | ladinin | −0.77 | 1.71E−02 |
| 11747 | Anxa5 | annexin A5 | −0.30 | 1.72E−02 |
| 380712 | Tlcd2 | TLC domain containing 2 | −0.42 | 1.73E−02 |
| 70575 | Gfod2 | glucose-fructose oxidoreductase domain containing 2 | −0.52 | 1.75E−02 |
| 19130 | Prox1 | prospero homeobox 1 | −0.51 | 1.78E−02 |
| 433702 | Ncbp1 | nuclear cap binding protein subunit 1 | −0.33 | 1.80E−02 |
| 53317 | Plrg1 | pleiotropic regulator 1 | −0.32 | 1.83E−02 |
| 66964 | Golt1b | golgi transport 1B | −0.56 | 1.87E−02 |
| 51886 | Fubp1 | far upstream element (FUSE) binding protein 1 | −0.32 | 1.87E−02 |
| 105148 | Iars | isoleucine-tRNA synthetase | −0.31 | 1.89E−02 |
| 13058 | Cybb | cytochrome b-245, beta polypeptide | −0.43 | 1.90E−02 |
| 98496 | Pid1 | phosphotyrosine interaction domain containing 1 | −0.27 | 1.91E−02 |
| 16828 | Ldha | lactate dehydrogenase A | −0.25 | 1.96E−02 |
| 12014 | Bach2 | BTB and CNC homology, basic leucine zipper transcription | −0.84 | 1.96E−02 |
| | | factor 2 | | |
| 241877 | Slc10a5 | solute carrier family 10 (sodium/bile acid cotransporter family), | −0.27 | 2.00E−02 |
| | | member 5 | | |
| 15950 | Ifi203 | interferon activated gene 203 | −0.47 | 2.14E−02 |
| 83797 | Smarcd1 | SWI/SNF related, matrix associated, actin dependent | −0.75 | 2.14E−02 |
| | | regulator of chromatin, subfamily d, member 1 | | |
| 59047 | Pnkp | polynucleotide kinase 3′- phosphatase | −0.28 | 2.14E−02 |
| 70601 | Ecd | ecdysoneless cell cycle regulator | −0.29 | 2.19E−02 |
| 15184 | Hdac5 | histone deacetylase 5 | −0.32 | 2.22E−02 |
| 73674 | Wdr75 | WD repeat domain 75 | −0.41 | 2.25E−02 |
| 246179 | Fktn | fukutin | −0.36 | 2.30E−02 |
| 13123 | Cyp7b1 | cytochrome P450, family 7, subfamily b, polypeptide 1 | −0.51 | 2.30E−02 |
| 80289 | Lysmd3 | LysM, putative peptidoglycan-binding, domain containing 3 | −0.33 | 2.30E−02 |
| 272538 | Tango6 | transport and golgi organization 6 | −0.42 | 2.31E−02 |
| 17164 | Mapkapk2 | MAP kinase-activated protein kinase 2 | −0.29 | 2.31E−02 |
| 12550 | Cdh1 | cadherin 1 | −0.64 | 2.42E−02 |
| 13107 | Cyp2f2 | cytochrome P450, family 2, subfamily f, polypeptide 2 | −0.39 | 2.45E−02 |
| 269704 | Zfp664 | zinc finger protein 664 | −0.36 | 2.47E−02 |
| 75964 | Trappc8 | trafficking protein particle complex 8 | −0.28 | 2.71E−02 |
| 74008 | Arsg | arylsulfatase G | −0.38 | 2.71E−02 |
| 20851 | Stat5b | signal transducer and activator of transcription 5B | −0.30 | 2.83E−02 |
| 71664 | Mettl7b | methyltransferase like 7B | −0.22 | 2.85E−02 |
| 52398 | Septin11 | septin 11 | −0.38 | 2.90E−02 |
| 232086 | Tmem150a | transmembrane protein 150A | −0.26 | 2.91E−02 |
| 320213 | Senp5 | SUMO/sentrin specific peptidase 5 | −0.31 | 2.96E−02 |
| 330401 | Tmcc1 | transmembrane and coiled coil domains 1 | −0.32 | 3.03E−02 |
| 268783 | Mtmr12 | myotubularin related protein 12 | −0.40 | 3.04E−02 |
| 14725 | Lrp2 | low density lipoprotein receptor-related protein 2 | −1.21 | 3.06E−02 |
| 66071 | Ethe1 | ethylmalonic encephalopathy 1 | −0.28 | 3.08E−02 |
| 101476 | Plekha1 | pleckstrin homology domain containing, family A | −0.37 | 3.12E−02 |
| | | (phosphoinositide binding specific) member 1 | | |
| 114332 | Lyve1 | lymphatic vessel endothelial hyaluronan receptor 1 | −0.64 | 3.12E−02 |
| 228998 | Arfgap1 | ADP-ribosylation factor GTPase activating protein 1 | −0.30 | 3.12E−02 |
| 105835 | Sgsm3 | small G protein signaling modulator 3 | −0.42 | 3.12E−02 |
| 16646 | Kpna1 | karyopherin (importin) alpha 1 | −0.27 | 3.15E−02 |
| 13436 | Dnmt3b | DNA methyltransferase 3B | −0.78 | 3.15E−02 |
| 27267 | Cars | cysteinyl-tRNA synthetase | −0.35 | 3.20E−02 |
| 20606 | Sstr2 | somatostatin receptor 2 | −0.17 | 3.21E−02 |
| 19212 | Pter | phosphotriesterase related | −0.30 | 3.23E−02 |
| 27054 | Sec23b | SEC23 homolog B, COPII coat complex component | −0.39 | 3.29E−02 |
| 240327 | Gm4951 | predicted gene 4951 | −0.23 | 3.29E−02 |
| 107503 | Atf5 | activating transcription factor 5 | −0.39 | 3.30E−02 |
| 52614 | Adgre4 | adhesion G protein-coupled receptor E4 | −0.45 | 3.35E−02 |
| 207375 | Fam120c | family with sequence similarity 120, member C | −0.52 | 3.36E−02 |
| 192176 | Flna | filamin, alpha | −0.34 | 3.39E−02 |
| 56095 | Ftsj3 | FtsJ RNA methyltransferase homolog 3 (E. coli) | −0.39 | 3.40E−02 |
| 71111 | Gpr39 | G protein-coupled receptor 39 | −0.61 | 3.54E−02 |
| 12317 | Calr | calreticulin | −0.27 | 3.68E−02 |
| 22145 | Tuba4a | tubulin, alpha 4A | −0.29 | 3.68E−02 |
| 235633 | Als2cl | ALS2 C-terminal like | −0.36 | 3.68E−02 |
| 228094 | Cerkl | ceramide kinase-like | −0.88 | 3.80E−02 |
| 110920 | Hspa13 | heat shock protein 70 family, member 13 | −0.30 | 3.84E−02 |
| 14020 | Evi5 | ecotropic viral integration site 5 | −0.22 | 3.84E−02 |
| 319583 | Lig4 | ligase IV, DNA, ATP-dependent | −0.52 | 3.84E−02 |
| 16401 | Itga4 | integrin alpha 4 | −0.50 | 3.85E−02 |
| 73692 | Cplane1 | ciliogenesis and planar polarity effector 1 | −0.50 | 3.86E−02 |
| 67298 | Gprasp1 | G protein-coupled receptor associated sorting protein 1 | −0.44 | 3.92E−02 |
| 76041 | Ccdc125 | coiled-coil domain containing 125 | −0.40 | 3.93E−02 |
| 11622 | Ahr | aryl-hydrocarbon receptor | −0.42 | 3.94E−02 |
| 56401 | P3h1 | prolyl 3-hydroxylase 1 | −0.39 | 3.95E−02 |
| 67226 | Tmem19 | transmembrane protein 19 | −0.23 | 3.95E−02 |
| 20720 | Serpine2 | serine (or cysteine) peptidase inhibitor, clade E, member 2 | −0.42 | 3.96E−02 |
| 69608 | Sec24d | Sec24 related gene family, member D (S. cerevisiae) | −0.31 | 4.01E−02 |
| 140740 | Sec63 | SEC63-like (S. cerevisiae) | −0.33 | 4.05E−02 |
| 140792 | Colec12 | collectin sub-family member 12 | −0.37 | 4.05E−02 |
| 109658 | Txlna | taxilin alpha | −0.30 | 4.08E−02 |
| 77065 | Ints7 | integrator complex subunit 7 | −0.36 | 4.13E−02 |
| 72748 | Hdhd3 | haloacid dehalogenase-like hydrolase domain containing 3 | −0.39 | 4.13E−02 |
| 15985 | Cd79b | CD79B antigen | −0.73 | 4.16E−02 |
| 225467 | Pggt1b | protein geranylgeranyltransferase type I, beta subunit | −0.37 | 4.23E−02 |
| 59126 | Nek6 | NIMA (never in mitosis gene a)-related expressed kinase 6 | −0.25 | 4.24E−02 |
| 74747 | Ddit4 | DNA-damage-inducible transcript 4 | −0.93 | 4.24E−02 |
| 11938 | Atp2a2 | ATPase, Ca++ transporting, cardiac muscle, slow twitch 2 | −0.39 | 4.26E−02 |
| 20747 | Spop | speckle-type BTB/POZ protein | −0.30 | 4.26E−02 |
| 50935 | St6galnac6 | ST6 (alpha-N-acetyl-neuraminyl-2,3-beta-galactosyl-1,3)-N- | −0.26 | 4.31E−02 |
| | | acetylgalactosaminide alpha-2,6-sialyltransferase 6 | | |
| 67512 | Agpat2 | 1-acylglycerol-3-phosphate O-acyltransferase 2 | −0.32 | 4.32E−02 |
| | | (lysophosphatidic acid acyltransferase, beta) | | |
| 100201 | Tmem64 | transmembrane protein 64 | −0.26 | 4.36E−02 |
| 170625 | Snx18 | sorting nexin 18 | −0.33 | 4.44E−02 |
| 236732 | Rbm10 | RNA binding motif protein 10 | −0.29 | 4.46E−02 |
| 22402 | Ccn4 | cellular communication network factor 4 | −0.60 | 4.46E−02 |
| 230126 | Shb | src homology 2 domain-containing transforming protein B | −0.42 | 4.47E−02 |
| 211389 | Suox | sulfite oxidase | −0.27 | 4.53E−02 |
| 72141 | Adpgk | ADP-dependent glucokinase | −0.32 | 4.60E−02 |
| 14043 | Ext2 | exostosin glycosyltransferase 2 | −0.27 | 4.62E−02 |
| 107338 | Gbf1 | golgi-specific brefeldin A-resistance factor 1 | −0.38 | 4.62E−02 |
| 57748 | Jmy | junction-mediating and regulatory protein | −0.40 | 4.65E−02 |
| 29876 | Clic4 | chloride intracellular channel 4 (mitochondrial) | −0.25 | 4.72E−02 |
| 78925 | Srd5a1 | steroid 5 alpha-reductase 1 | −0.32 | 4.73E−02 |
| 15500 | Hsf2 | heat shock factor 2 | −0.29 | 4.74E−02 |
| 224273 | Crybg3 | beta-gamma crystallin domain containing 3 | −0.43 | 4.75E−02 |
| 13864 | Nr2f6 | nuclear receptor subfamily 2, group F, member 6 | −0.27 | 4.75E−02 |
| 226105 | Cyp2c70 | cytochrome P450, family 2, subfamily c, polypeptide 70 | −0.40 | 4.75E−02 |
| 102098 | Arhgef18 | rho/rac guanine nucleotide exchange factor (GEF) 18 | −0.33 | 4.75E−02 |
| 112407 | Egln3 | egl-9 family hypoxia-inducible factor 3 | −0.34 | 4.75E−02 |
| 11432 | Acp2 | acid phosphatase 2, lysosomal | −0.24 | 4.76E−02 |
| 108114 | Slc22a7 | solute carrier family 22 (organic anion transporter), member 7 | −0.53 | 4.84E−02 |
| 67230 | Zfp329 | zinc finger protein 329 | −0.57 | 4.84E−02 |
| 14583 | Gfpt1 | glutamine fructose-6-phosphate transaminase 1 | −0.39 | 4.89E−02 |
| 15568 | Elavl1 | ELAV (embryonic lethal, abnormal vision)-like 1 (Hu antigen R) | −0.32 | 4.89E−02 |
| 71653 | Shtn1 | shootin 1 | −0.24 | 4.92E−02 |
| 74178 | Stk40 | serine/threonine kinase 40 | −0.37 | 4.94E−02 |

TABLE S3C
Upregulated genes in colon.

| | | | | FDR- |
|---|---|---|---|---|
| | | | | adjusted |
| Entrez | | | log2 fold- | Wald test |
| Gene ID | Symbol | Name | change | P-value |
| 319482 | 9530053A07Rik | RIKEN cDNA 9530053A07 gene | 10.44 | 7.08E−10 |
| 18628 | Per3 | period circadian clock 3 | 1.17 | 9.67E−10 |
| 331046 | Tgm4 | transglutaminase 4 (prostate) | 9.55 | 3.63E−09 |
| 235320 | Zbtb16 | zinc finger and BTB domain containing 16 | 1.21 | 2.85E−07 |
| 494448 | Cbx6 | chromobox 6 | 0.65 | 7.03E−07 |
| 330814 | Adgrl1 | adhesion G protein-coupled receptor L1 | 0.82 | 9.17E−07 |
| 26949 | Vat1 | vesicle amine transport 1 | 0.64 | 2.51E−06 |
| 192734 | Lrrc75b | leucine rich repeat containing 75B | 2.59 | 6.64E−06 |
| 13170 | Dbp | D site albumin promoter binding protein | 1.14 | 7.52E−06 |
| 16011 | Igfbp5 | insulin-like growth factor binding protein 5 | 0.78 | 1.05E−05 |
| 216505 | Pik3ip1 | phosphoinositide-3-kinase interacting protein 1 | 1.04 | 1.09E−05 |
| 217166 | Nr1d1 | nuclear receptor subfamily 1, group D, member 1 | 0.81 | 1.69E−05 |
| 403088 | Tcaf3 | TRPM8 channel-associated factor 3 | 11.09 | 1.82E−05 |
| 53878 | Svs2 | seminal vesicle secretory protein 2 | 8.79 | 2.86E−05 |
| 243377 | Svs1 | seminal vesicle secretory protein 1 | 8.01 | 2.93E−05 |
| 54192 | Pbsn | probasin | 10.71 | 3.61E−05 |
| 19701 | Ren1 | renin 1 structural | 8.82 | 3.64E−05 |
| 269063 | Ms4a5 | membrane-spanning 4-domains, subfamily A, | 3.19 | 3.86E−05 |
| | | member 5 | | |
| 17750 | Mt2 | metallothionein 2 | 0.75 | 4.12E−05 |
| 14711 | Gnmt | glycine N-methyltransferase | 3.61 | 4.22E−05 |
| 217082 | Hlf | hepatic leukemia factor | 1.26 | 5.27E−05 |
| 26366 | Ceacam10 | carcinoembryonic antigen-related cell adhesion | 5.61 | 7.67E−05 |
| | | molecule 10 | | |
| 209039 | Tns2 | tensin 2 | 0.57 | 7.78E−05 |
| 20944 | Svs5 | seminal vesicle secretory protein 5 | 7.81 | 9.23E−05 |
| 272636 | Esyt3 | extended synaptotagmin-like protein 3 | 3.43 | 9.47E−05 |
| 619548 | Defb42 | defensin beta 42 | 6.02 | 1.01E−04 |
| 20941 | Svs4 | seminal vesicle secretory protein 4 | 8.34 | 1.04E−04 |
| 14461 | Gata2 | GATA binding protein 2 | 5.30 | 1.17E−04 |
| 667277 | C1rb | complement component 1, r subcomponent B | 7.69 | 1.20E−04 |
| 269855 | Ssc5d | scavenger receptor cysteine rich family, 5 | 0.94 | 1.52E−04 |
| | | domains | | |
| 18491 | Pappa | pregnancy-associated plasma protein A | 0.60 | 1.60E−04 |
| 230810 | Slc30a2 | solute carrier family 30 (zinc transporter), | 3.33 | 1.87E−04 |
| | | member 2 | | |
| 330790 | Hapln4 | hyaluronan and proteoglycan link protein 4 | 3.45 | 3.28E−04 |
| 329557 | Svs3b | seminal vesicle secretory protein 3B | 8.03 | 3.76E−04 |
| 74525 | Fam234b | family with sequence similarity 234, member B | 1.49 | 3.91E−04 |
| 51789 | Tnk2 | tyrosine kinase, non-receptor, 2 | 0.56 | 4.34E−04 |
| 14264 | Fmod | fibromodulin | 1.91 | 4.48E−04 |
| 214663 | Slc25a29 | solute carrier family 25 (mitochondrial carrier, | 1.25 | 4.49E−04 |
| | | palmitoylcarnitine transporter), member 29 | | |
| 16776 | Lama5 | laminin, alpha 5 | 0.51 | 4.59E−04 |
| 16779 | Lamb2 | laminin, beta 2 | 0.47 | 4.93E−04 |
| 20945 | Svs6 | seminal vesicle secretory protein 6 | 7.66 | 4.97E−04 |
| 94216 | Col4a6 | collagen, type IV, alpha 6 | 1.01 | 6.01E−04 |
| 68617 | Mtcl1 | microtubule crosslinking factor 1 | 0.66 | 6.01E−04 |
| 66277 | Klf15 | Kruppel-like factor 15 | 1.38 | 6.01E−04 |
| 22673 | Zfp185 | zinc finger protein 185 | 2.32 | 6.09E−04 |
| 93960 | Nkd1 | naked cuticle 1 | 0.80 | 7.23E−04 |
| 77432 | 9530002B09Rik | RIKEN cDNA 9530002B09 gene | 9.07 | 7.60E−04 |
| 13982 | Esr1 | estrogen receptor 1 (alpha) | 1.09 | 7.87E−04 |
| 12288 | Cacna1c | calcium channel, voltage-dependent, L type, | 0.54 | 8.04E−04 |
| | | alpha 1C subunit | | |
| 64335 | Svs3a | seminal vesicle secretory protein 3A | 7.68 | 8.12E−04 |
| 81877 | Tnxb | tenascin XB | 0.50 | 8.20E−04 |
| 373864 | Col27a1 | collagen, type XXVII, alpha 1 | 0.94 | 9.79E−04 |
| 80290 | Gpr146 | G protein-coupled receptor 146 | 0.73 | 1.06E−03 |
| 68859 | Smim1 | small integral membrane protein 1 | 1.51 | 1.06E−03 |
| 30937 | Lmcd1 | LIM and cysteine-rich domains 1 | 1.45 | 1.12E−03 |
| 21804 | Tgfb1i1 | transforming growth factor beta 1 induced | 0.54 | 1.16E−03 |
| | | transcript 1 | | |
| 14269 | Fnbp1 | formin binding protein 1 | 0.46 | 1.17E−03 |
| 52897 | Rbfox3 | RNA binding protein, fox-1 homolog (C. elegans) | 2.79 | 1.18E−03 |
| | | 3 | | |
| 17428 | Mnt | max binding protein | 0.76 | 1.21E−03 |
| 14049 | Eya2 | EYA transcriptional coactivator and phosphatase | 2.05 | 1.23E−03 |
| | | 2 | | |
| 17153 | Mal | myelin and lymphocyte protein, T cell | 0.85 | 1.32E−03 |
| | | differentiation protein | | |
| 320924 | Ccbe1 | collagen and calcium binding EGF domains 1 | 1.10 | 1.33E−03 |
| 18032 | Nfix | nuclear factor I/X | 0.64 | 1.42E−03 |
| 17929 | Myom1 | myomesin 1 | 0.49 | 1.42E−03 |
| 195733 | Grhl1 | grainyhead like transcription factor 1 | 1.31 | 1.42E−03 |
| 207740 | Ubald1 | UBA-like domain containing 1 | 0.67 | 1.49E−03 |
| 18481 | Pak3 | p21 (RAC1) activated kinase 3 | 0.88 | 1.64E−03 |
| 227394 | Slco4c1 | solute carrier organic anion transporter family, | 5.14 | 1.76E−03 |
| | | member 4C1 | | |
| 14227 | Fkbp2 | FK506 binding protein 2 | 0.56 | 1.76E−03 |
| 21987 | Tpd52l1 | tumor protein D52-like 1 | 1.19 | 1.76E−03 |
| 70893 | Glb1l3 | galactosidase, beta 1 like 3 | 10.56 | 1.87E−03 |
| 83669 | Wdr6 | WD repeat domain 6 | 0.46 | 1.87E−03 |
| 26360 | Angptl2 | angiopoietin-like 2 | 0.49 | 1.87E−03 |
| 110308 | Krt5 | keratin 5 | 6.22 | 1.87E−03 |
| 232313 | Gxylt2 | glucoside xylosyltransferase 2 | 1.98 | 1.89E−03 |
| 226778 | Mark1 | MAP/microtubule affinity regulating kinase 1 | 0.81 | 2.29E−03 |
| 13048 | Cux2 | cut-like homeobox 2 | 1.61 | 2.41E−03 |
| 17393 | Mmp7 | matrix metallopeptidase 7 | 4.17 | 2.54E−03 |
| 11684 | Alox12 | arachidonate 12-lipoxygenase | 2.13 | 2.61E−03 |
| 75665 | Bicdl1 | BICD family like cargo adaptor 1 | 0.64 | 2.64E−03 |
| 17181 | Matn2 | matrilin 2 | 0.69 | 2.64E−03 |
| 53322 | Nucb2 | nucleobindin 2 | 3.53 | 2.73E−03 |
| 235493 | Fam214a | family with sequence similarity 214, member A | 0.49 | 2.76E−03 |
| 107503 | Atf5 | activating transcription factor 5 | 0.70 | 2.90E−03 |
| 73296 | Rhobtb3 | Rho-related BTB domain containing 3 | 0.86 | 2.91E−03 |
| 207181 | Rbms3 | RNA binding motif, single stranded interacting | 0.45 | 3.04E−03 |
| | | protein | | |
| 56233 | Hdac7 | histone deacetylase 7 | 0.63 | 3.16E−03 |
| 14560 | Gdf10 | growth differentiation factor 10 | 0.91 | 3.27E−03 |
| 329217 | Panct2 | pluripotency-associated noncoding transcript 2 | 1.72 | 3.46E−03 |
| 23917 | Impdh1 | inosine monophosphate dehydrogenase 1 | 0.53 | 3.56E−03 |
| 64292 | Ptges | prostaglandin E synthase | 1.14 | 3.64E−03 |
| 12696 | Cirbp | cold inducible RNA binding protein | 0.65 | 3.75E−03 |
| 170460 | Stard5 | StAR-related lipid transfer (START) domain | 0.43 | 3.76E−03 |
| | | containing 5 | | |
| 215798 | Adgrg6 | adhesion G protein-coupled receptor G6 | 1.06 | 3.77E−03 |
| 22262 | Uox | urate oxidase | 2.40 | 3.98E−03 |
| 327958 | Pitpnm3 | PITPNM family member 3 | 0.67 | 3.98E−03 |
| 56872 | Pate4 | prostate and testis expressed 4 | 7.55 | 4.03E−03 |
| 18792 | Plau | plasminogen activator, urokinase | 0.93 | 4.03E−03 |
| 26968 | Islr | immunoglobulin superfamily containing leucine- | 0.52 | 4.03E−03 |
| | | rich repeat | | |
| 77424 | Spinkl | serine protease inhibitor, Kazal type-like | 7.58 | 4.29E−03 |
| 17174 | Masp1 | mannan-binding lectin serine peptidase 1 | 1.38 | 4.63E−03 |
| 217335 | Fbf1 | Fas (TNFRSF6) binding factor 1 | 0.51 | 4.64E−03 |
| 20317 | Serpinf1 | serine (or cysteine) peptidase inhibitor, clade F, | 0.82 | 4.71E−03 |
| | | member 1 | | |
| 80708 | Pacsin3 | protein kinase C and casein kinase substrate in | 0.87 | 4.77E−03 |
| | | neurons 3 | | |
| 238057 | Gdf7 | growth differentiation factor 7 | 2.66 | 4.87E−03 |
| 74126 | Syvn1 | synovial apoptosis inhibitor 1, synoviolin | 0.58 | 4.88E−03 |
| 226519 | Lamc1 | laminin, gamma 1 | 0.37 | 5.23E−03 |
| 57911 | Gsdma | gasdermin A | 3.93 | 5.27E−03 |
| 18160 | Npr1 | natriuretic peptide receptor 1 | 0.99 | 5.41E−03 |
| 70377 | Derl3 | Der1-like domain family, member 3 | 3.02 | 5.42E−03 |
| 319622 | Itpripl2 | inositol 1,4,5-triphosphate receptor interacting | 0.42 | 5.59E−03 |
| | | protein-like 2 | | |
| 94214 | Spock2 | sparc/osteonectin, cwcv and kazal-like domains | 0.54 | 5.99E−03 |
| | | proteoglycan 2 | | |
| 103551 | Epop | elongin BC and polycomb repressive complex 2 | 1.75 | 6.15E−03 |
| | | associated protein | | |
| 18993 | Pou3f3 | POU domain, class 3, transcription factor 3 | 1.18 | 6.18E−03 |
| 17427 | Mns1 | meiosis-specific nuclear structural protein 1 | 1.12 | 6.21E−03 |
| 68775 | Atp6v1c2 | ATPase, H+ transporting, lysosomal V1 subunit | 5.46 | 6.52E−03 |
| | | C2 | | |
| 71774 | Shroom1 | shroom family member 1 | 2.33 | 6.60E−03 |
| 80288 | Bcl9l | B cell CLL/lymphoma 9-like | 0.57 | 6.96E−03 |
| 320145 | Sp8 | trans-acting transcription factor 8 | 2.57 | 7.04E−03 |
| 19672 | Rcn1 | reticulocalbin 1 | 2.72 | 7.04E−03 |
| 21814 | Tgfbr3 | transforming growth factor, beta receptor III | 0.43 | 7.04E−03 |
| 30956 | Aass | aminoadipate-semialdehyde synthase | 4.35 | 7.04E−03 |
| 20741 | Sptb | spectrin beta, erythrocytic | 0.85 | 7.08E−03 |
| 15438 | Hoxd9 | homeobox D9 | 1.18 | 7.15E−03 |
| 17341 | Bhlha15 | basic helix-loop-helix family, member a15 | 2.54 | 7.23E−03 |
| 53761 | Prrc2a | proline-rich coiled-coil 2A | 0.49 | 7.27E−03 |
| 11906 | Zfhx3 | zinc finger homeobox 3 | 0.54 | 7.49E−03 |
| 20720 | Serpine2 | serine (or cysteine) peptidase inhibitor, clade E, | 2.50 | 7.57E−03 |
| | | member 2 | | |
| 22268 | Upk1b | uroplakin 1B | 2.61 | 7.75E−03 |
| 234797 | 6430548M08Rik | RIKEN cDNA 6430548M08 gene | 0.47 | 7.75E−03 |
| 216188 | Aldh1l2 | aldehyde dehydrogenase 1 family, member L2 | 1.36 | 7.97E−03 |
| 74761 | Mxra8 | matrix-remodelling associated 8 | 0.45 | 8.08E−03 |
| 246228 | Vwa1 | von Willebrand factor A domain containing 1 | 0.64 | 8.19E−03 |
| 56629 | Dnase2b | deoxyribonuclease II beta | 6.74 | 8.32E−03 |
| 207212 | Arhgef17 | Rho guanine nucleotide exchange factor (GEF) | 0.43 | 8.42E−03 |
| | | 17 | | |
| 16450 | Jag2 | jagged 2 | 0.92 | 8.42E−03 |
| 16664 | Krt14 | keratin 14 | 2.57 | 8.42E−03 |
| 381260 | Gm973 | predicted gene 973 | 1.71 | 8.42E−03 |
| 18095 | Nkx3-1 | NK3 homeobox 1 | 0.01 | 8.61E−03 |
| 243376 | Doxl2 | diamine oxidase-like protein 2 | 0.01 | 9.39E−03 |
| 71790 | Anxa9 | annexin A9 | 3.41 | 9.67E−03 |
| 29867 | Cabp1 | calcium binding protein 1 | 2.18 | 1.02E−02 |
| 12007 | Azgp1 | alpha-2-glycoprotein 1, zinc | 3.05 | 1.05E−02 |
| 21961 | Tns1 | tensin 1 | 0.44 | 1.06E−02 |
| 59033 | Slc4a8 | solute carrier family 4 (anion exchanger), | 1.04 | 1.07E−02 |
| | | member 8 | | |
| 56739 | Rec8 | REC8 meiotic recombination protein | 1.38 | 1.08E−02 |
| 330938 | Dixdc1 | DIX domain containing 1 | 0.49 | 1.08E−02 |
| 20522 | Slc23a1 | solute carrier family 23 (nucleobase | 1.80 | 1.08E−02 |
| | | transporters), member 1 | | |
| 434396 | Pate9 | prostate and testis expressed 9 | 0.01 | 1.09E−02 |
| 244654 | Mtss2 | MTSS I-BAR domain containing 2 | 0.61 | 1.09E−02 |
| 18627 | Per2 | period circadian clock 2 | 0.67 | 1.10E−02 |
| 12822 | Col18a1 | collagen, type XVIII, alpha 1 | 0.40 | 1.14E−02 |
| 230806 | Crybg2 | crystallin beta-gamma domain containing 2 | 0.56 | 1.14E−02 |
| 99663 | Clca4a | chloride channel accessory 4A | 0.41 | 1.17E−02 |
| 317677 | C1s2 | complement component 1, s subcomponent 2 | 6.81 | 1.18E−02 |
| 108100 | Baiap2 | brain-specific angiogenesis inhibitor 1- | 0.55 | 1.21E−02 |
| | | associated protein 2 | | |
| 140481 | Man2a2 | mannosidase 2, alpha 2 | 0.35 | 1.21E−02 |
| 330409 | Cecr2 | CECR2, histone acetyl-lysine reader | 1.23 | 1.21E−02 |
| 234199 | Fgl1 | fibrinogen-like protein 1 | 3.75 | 1.22E−02 |
| 14853 | Gspt2 | G1 to S phase transition 2 | 1.14 | 1.22E−02 |
| 223864 | Rapgef3 | Rap guanine nucleotide exchange factor (GEF) | 0.54 | 1.22E−02 |
| | | 3 | | |
| 226041 | Pgm5 | phosphoglucomutase 5 | 0.29 | 1.25E−02 |
| 20939 | Sva | seminal vesicle antigen | 6.86 | 1.26E−02 |
| 216850 | Kdm6b | KDM1 lysine (K)-specific demethylase 6B | 0.57 | 1.26E−02 |
| 235973 | Pate14 | prostate and testis expressed 14 | 6.47 | 1.28E−02 |
| 50722 | Dkkl1 | dickkopf-like 1 | 4.99 | 1.28E−02 |
| 224090 | Tmem44 | transmembrane protein 44 | 0.56 | 1.28E−02 |
| 70355 | Gprc5c | G protein-coupled receptor, family C, group 5, | 2.14 | 1.33E−02 |
| | | member C | | |
| 75580 | Zbtb4 | zinc finger and BTB domain containing 4 | 0.37 | 1.33E−02 |
| 213121 | Ankrd35 | ankyrin repeat domain 35 | 1.27 | 1.37E−02 |
| 52882 | Rgs7bp | regulator of G-protein signalling 7 binding protein | 0.57 | 1.40E−02 |
| 237759 | Col23a1 | collagen, type XXIII, alpha 1 | 0.53 | 1.41E−02 |
| 77397 | 9530003J23Rik | RIKEN cDNA 9530003J23 gene | 7.07 | 1.41E−02 |
| 66972 | Slc25a23 | solute carrier family 25 (mitochondrial carrier; | 0.54 | 1.43E−02 |
| | | phosphate carrier), member 23 | | |
| 229599 | Ciart | circadian associated repressor of transcription | 1.10 | 1.43E−02 |
| 77552 | Shisa4 | shisa family member 4 | 2.78 | 1.43E−02 |
| 13007 | Csrp1 | cysteine and glycine-rich protein 1 | 0.39 | 1.43E−02 |
| 26422 | Nbea | neurobeachin | 0.35 | 1.44E−02 |
| 353187 | Nr1d2 | nuclear receptor subfamily 1, group D, member | 0.42 | 1.44E−02 |
| | | 2 | | |
| 13644 | Efs | embryonal Fyn-associated substrate | 0.99 | 1.45E−02 |
| 70445 | Cd248 | CD248 antigen, endosialin | 0.77 | 1.45E−02 |
| 17294 | Mest | mesoderm specific transcript | 0.81 | 1.47E−02 |
| 22404 | Wiz | widely-interspaced zinc finger motifs | 0.55 | 1.47E−02 |
| 20564 | Slit3 | slit guidance ligand 3 | 0.45 | 1.49E−02 |
| 319262 | Fchsd1 | FCH and double SH3 domains 1 | 0.90 | 1.50E−02 |
| 100043911 | Ppp4r1l-ps | protein phosphatase 4, regulatory subunit 1-like, | 0.68 | 1.59E−02 |
| | | pseudogene | | |
| 50873 | Prkn | parkin RBR E3 ubiquitin protein ligase | 1.09 | 1.67E−02 |
| 109624 | Cald1 | caldesmon 1 | 0.42 | 1.67E−02 |
| 13711 | Elf5 | E74-like factor 5 | 0.01 | 1.75E−02 |
| 16001 | Igf1r | insulin-like growth factor I receptor | 0.45 | 1.75E−02 |
| 13528 | Dtnb | dystrobrevin, beta | 0.79 | 1.78E−02 |
| 93843 | Pnck | pregnancy upregulated non-ubiquitously | 0.85 | 1.85E−02 |
| | | expressed CaM kinase | | |
| 108653 | Rimklb | ribosomal modification protein rimK-like family | 2.24 | 1.88E−02 |
| | | member B | | |
| 12167 | Bmpr1b | bone morphogenetic protein receptor, type 1B | 3.17 | 1.88E−02 |
| 225998 | Rorb | RAR-related orphan receptor beta | 1.88 | 1.91E−02 |
| 329251 | Ppp1r12b | protein phosphatase 1, regulatory subunit 12B | 0.27 | 1.92E−02 |
| 74376 | Myo18b | myosin XVIIIb | 1.18 | 1.92E−02 |
| 223918 | Spryd3 | SPRY domain containing 3 | 0.38 | 1.94E−02 |
| 56695 | Pnkd | paroxysmal nonkinesiogenic dyskinesia | 0.53 | 2.03E−02 |
| 98402 | Sh3bp4 | SH3-domain binding protein 4 | 0.55 | 2.05E−02 |
| 109593 | Lmo3 | LIM domain only 3 | 2.43 | 2.07E−02 |
| 13645 | Egf | epidermal growth factor | 2.10 | 2.13E−02 |
| 53376 | Usp2 | ubiquitin specific peptidase 2 | 0.51 | 2.14E−02 |
| 94191 | Adarb2 | adenosine deaminase, RNA-specific, B2 | 2.23 | 2.14E−02 |
| 209027 | Pycr1 | pyrroline-5-carboxylate reductase 1 | 0.88 | 2.16E−02 |
| 78309 | Cul9 | cullin 9 | 0.49 | 2.16E−02 |
| 665700 | Hmcn2 | hemicentin 2 | 0.39 | 2.17E−02 |
| 192212 | Prom2 | prominin 2 | 0.01 | 2.17E−02 |
| 192197 | Bcas3 | breast carcinoma amplified sequence 3 | 0.38 | 2.19E−02 |
| 76872 | Ccdc116 | coiled-coil domain containing 116 | 0.62 | 2.22E−02 |
| 74309 | Osbp2 | oxysterol binding protein 2 | 1.55 | 2.23E−02 |
| 242785 | Klhl21 | kelch-like 21 | 0.38 | 2.23E−02 |
| 16008 | Igfbp2 | insulin-like growth factor binding protein 2 | 0.75 | 2.34E−02 |
| 70427 | Mier2 | MIER family member 2 | 0.47 | 2.36E−02 |
| 79362 | Bhlhe41 | basic helix-loop-helix family, member e41 | 0.97 | 2.45E−02 |
| 18946 | Pnliprp1 | pancreatic lipase related protein 1 | 3.29 | 2.46E−02 |
| 226518 | Nmnat2 | nicotinamide nucleotide adenylyltransferase 2 | 0.68 | 2.46E−02 |
| 19280 | Ptprs | protein tyrosine phosphatase, receptor type, S | 0.43 | 2.46E−02 |
| 29818 | Hspb7 | heat shock protein family, member 7 | 0.62 | 2.46E−02 |
| | | (cardiovascular) | | |
| 230757 | 5730409E04Rik | RIKEN cDNA 5730409E04Rik gene | 0.73 | 2.47E−02 |
| 12346 | Car1 | carbonic anhydrase 1 | 0.44 | 2.49E−02 |
| 21366 | Slc6a6 | solute carrier family 6 (neurotransmitter | 0.32 | 2.49E−02 |
| | | transporter, taurine), member 6 | | |
| 71889 | Epn3 | epsin 3 | 0.42 | 2.49E−02 |
| 56312 | Nupr1 | nuclear protein transcription regulator 1 | 0.41 | 2.49E−02 |
| 233908 | Fus | fused in sarcoma | 0.57 | 2.52E−02 |
| 13447 | Doc2b | double C2, beta | 1.05 | 2.53E−02 |
| 114249 | Npnt | nephronectin | 0.51 | 2.53E−02 |
| 67216 | Mboat2 | membrane bound O-acyltransferase domain | 0.45 | 2.56E−02 |
| | | containing 2 | | |
| 11459 | Acta1 | actin, alpha 1, skeletal muscle | 1.78 | 2.57E−02 |
| 277154 | Nynrin | NYN domain and retroviral integrase containing | 0.50 | 2.57E−02 |
| 19679 | Pitpnm2 | phosphatidylinositol transfer protein, membrane- | 0.48 | 2.62E−02 |
| | | associated 2 | | |
| 214384 | Myocd | myocardin | 0.40 | 2.66E−02 |
| 225115 | Svil | supervillin | 0.29 | 2.67E−02 |
| 269401 | Zfp512b | zinc finger protein 512B | 0.42 | 2.70E−02 |
| 72289 | Malat1 | metastasis associated lung adenocarcinoma | 0.24 | 2.81E−02 |
| | | transcript 1 (non-coding RNA) | | |
| 13800 | Enah | ENAH actin regulator | 0.50 | 2.83E−02 |
| 20404 | Sh3gl2 | SH3-domain GRB2-like 2 | 0.49 | 2.86E−02 |
| 58226 | Cacna1h | calcium channel, voltage-dependent, T type, | 0.41 | 2.86E−02 |
| | | alpha 1H subunit | | |
| 57914 | Crlf2 | cytokine receptor-like factor 2 | 0.72 | 2.86E−02 |
| 231830 | Micall2 | MICAL-like 2 | 0.53 | 2.89E−02 |
| 98396 | Slc41a1 | solute carrier family 41, member 1 | 0.38 | 2.90E−02 |
| 237979 | Sdk2 | sidekick cell adhesion molecule 2 | 0.72 | 2.94E−02 |
| 12583 | Cdo1 | cysteine dioxygenase 1, cytosolic | 1.77 | 2.94E−02 |
| 99543 | Olfml3 | olfactomedin-like 3 | 0.45 | 2.95E−02 |
| 26562 | Ncdn | neurochondrin | 0.51 | 2.95E−02 |
| 64085 | Clstn2 | calsyntenin 2 | 1.12 | 2.95E−02 |
| 215445 | Rab11fip3 | RAB11 family interacting protein 3 (class II) | 0.48 | 2.98E−02 |
| 13650 | Rhbdf1 | rhomboid 5 homolog 1 | 0.47 | 3.02E−02 |
| 20544 | Slc9a1 | solute carrier family 9 (sodium/hydrogen | 0.45 | 3.04E−02 |
| | | exchanger), member 1 | | |
| 19752 | Rnase1 | ribonuclease, RNase A family, 1 (pancreatic) | 3.42 | 3.08E−02 |
| 73338 | Itpripl1 | inositol 1,4,5-triphosphate receptor interacting | 0.78 | 3.10E−02 |
| | | protein-like 1 | | |
| 16012 | Igfbp6 | insulin-like growth factor binding protein 6 | 0.81 | 3.10E−02 |
| 259302 | Srgap3 | SLIT-ROBO Rho GTPase activating protein 3 | 0.41 | 3.12E−02 |
| 21386 | Tbx3 | T-box 3 | 1.10 | 3.12E−02 |
| 11987 | Slc7a1 | solute carrier family 7 (cationic amino acid | 0.52 | 3.13E−02 |
| | | transporter, y+ system), member 1 | | |
| 20502 | Slc16a2 | solute carrier family 16 (monocarboxylic acid | 0.73 | 3.17E−02 |
| | | transporters), member 2 | | |
| 208285 | Cyp4f17 | cytochrome P450, family 4, subfamily f, | 0.91 | 3.17E−02 |
| | | polypeptide 17 | | |
| 12325 | Camk2g | calcium/calmodulin-dependent protein kinase II | 0.40 | 3.18E−02 |
| | | gamma | | |
| 333424 | A4gnt | alpha-1,4-N-acetylglucosaminyltransferase | 0.36 | 3.19E−02 |
| 93689 | Lmod1 | leiomodin 1 (smooth muscle) | 0.30 | 3.19E−02 |
| 100213 | Rusc2 | RUN and SH3 domain containing 2 | 0.43 | 3.23E−02 |
| 100416160 | Wdr46-ps | Wdr46 retrotransposed pseudogene | 1.52 | 3.24E−02 |
| 68519 | Eml1 | echinoderm microtubule associated protein like | 0.35 | 3.24E−02 |
| | | 1 | | |
| 246154 | Vasn | vasorin | 0.62 | 3.26E−02 |
| 330908 | Opcml | opioid binding protein/cell adhesion molecule- | 0.79 | 3.28E−02 |
| | | like | | |
| 94275 | Maged1 | MAGE family member D1 | 0.34 | 3.28E−02 |
| 71843 | R3hcc1 | R3H domain and coiled-coil containing 1 | 0.43 | 3.28E−02 |
| 230673 | Ipo13 | importin 13 | 0.44 | 3.28E−02 |
| 18933 | Prrx1 | paired related homeobox 1 | 1.42 | 3.28E−02 |
| 70310 | Plscr3 | phospholipid scramblase 3 | 0.55 | 3.28E−02 |
| 241727 | Snph | syntaphilin | 0.59 | 3.28E−02 |
| 329941 | Col8a2 | collagen, type VIII, alpha 2 | 0.74 | 3.28E−02 |
| 16998 | Ltbp3 | latent transforming growth factor beta binding | 0.48 | 3.29E−02 |
| | | protein 3 | | |
| 244152 | Tsku | tsukushi, small leucine rich proteoglycan | 0.48 | 3.31E−02 |
| 244757 | Glb1l2 | galactosidase, beta 1-like 2 | 1.79 | 3.31E−02 |
| 68337 | Crip2 | cysteine rich protein 2 | 0.46 | 3.31E−02 |
| 18407 | Orm3 | orosomucoid 3 | 0.01 | 3.31E−02 |
| 57276 | Vsig2 | V-set and immunoglobulin domain containing 2 | 0.69 | 3.32E−02 |
| 385658 | Nxpe3 | neurexophilin and PC-esterase domain family, | 1.84 | 3.35E−02 |
| | | member 3 | | |
| 66885 | Acadsb | acyl-Coenzyme A dehydrogenase, | 0.46 | 3.45E−02 |
| | | short/branched chain | | |
| 70393 | 2210416O15Rik | RIKEN cDNA 2210416O15 gene | 0.48 | 3.45E−02 |
| 14115 | Fbln2 | fibulin 2 | 0.35 | 3.49E−02 |
| 71722 | Cic | capicua transcriptional repressor | 0.36 | 3.49E−02 |
| 74694 | Tbc1d30 | TBC1 domain family, member 30 | 0.39 | 3.49E−02 |
| 320404 | Itpkb | inositol 1,4,5-trisphosphate 3-kinase B | 0.31 | 3.52E−02 |
| 228846 | D630003M21Rik | RIKEN cDNA D630003M21 gene | 0.74 | 3.56E−02 |
| 228140 | Tnks1bp1 | tankyrase 1 binding protein 1 | 0.36 | 3.56E−02 |
| 66961 | Neat1 | nuclear paraspeckle assembly transcript 1 (non- | 0.33 | 3.61E−02 |
| | | protein coding) | | |
| 55984 | Camkk1 | calcium/calmodulin-dependent protein kinase | 0.76 | 3.61E−02 |
| | | kinase 1, alpha | | |
| 68267 | Slc25a22 | solute carrier family 25 (mitochondrial carrier, | 0.52 | 3.62E−02 |
| | | glutamate), member 22 | | |
| 72543 | Mvb12b | multivesicular body subunit 12B | 0.38 | 3.65E−02 |
| 17988 | Ndrg1 | N-myc downstream regulated gene 1 | 0.33 | 3.65E−02 |
| 21887 | Tle3 | transducin-like enhancer of split 3 | 0.34 | 3.65E−02 |
| 16512 | Kcnh3 | potassium voltage-gated channel, subfamily H | 0.47 | 3.65E−02 |
| | | (eag-related), member 3 | | |
| 330010 | Ttll10 | tubulin tyrosine ligase-like family, member 10 | 0.60 | 3.66E−02 |
| 20503 | Slc16a7 | solute carrier family 16 (monocarboxylic acid | 1.26 | 3.67E−02 |
| | | transporters), member 7 | | |
| 20972 | Syngr1 | synaptogyrin 1 | 0.50 | 3.67E−02 |
| 329872 | Frem1 | Fras1 related extracellular matrix protein 1 | 0.80 | 3.69E−02 |
| 240518 | Peli3 | pellino 3 | 0.74 | 3.72E−02 |
| 93721 | Cpn1 | carboxypeptidase N, polypeptide 1 | 0.55 | 3.72E−02 |
| 18807 | Pld3 | phospholipase D family, member 3 | 0.35 | 3.73E−02 |
| 14275 | Folr1 | folate receptor 1 (adult) | 2.49 | 3.73E−02 |
| 218820 | Zfp503 | zinc finger protein 503 | 0.65 | 3.73E−02 |
| 50770 | Atp11a | ATPase, class VI, type 11A | 0.46 | 3.74E−02 |
| 320825 | Samd5 | sterile alpha motif domain containing 5 | 0.41 | 3.75E−02 |
| 108075 | Ltbp4 | latent transforming growth factor beta binding | 0.37 | 3.76E−02 |
| | | protein 4 | | |
| 29856 | Smtn | smoothelin | 0.31 | 3.76E−02 |
| 102693 | Phldb1 | pleckstrin homology like domain, family B, | 0.34 | 3.78E−02 |
| | | member 1 | | |
| 107975 | Pacs1 | phosphofurin acidic cluster sorting protein 1 | 0.44 | 3.78E−02 |
| 434130 | Ccdc8 | coiled-coil domain containing 8 | 0.83 | 3.80E−02 |
| 381217 | Fam189a2 | family with sequence similarity 189, member A2 | 0.32 | 3.86E−02 |
| 16979 | Lrrn1 | leucine rich repeat protein 1, neuronal | 1.45 | 3.92E−02 |
| 208043 | Setd1b | SET domain containing 1B | 0.49 | 3.95E−02 |
| 330286 | D630045J12Rik | RIKEN cDNA D630045J12 gene | 0.96 | 3.96E−02 |
| 109979 | Art3 | ADP-ribosyltransferase 3 | 0.40 | 3.96E−02 |
| 56485 | Slc2a5 | solute carrier family 2 (facilitated glucose | 0.65 | 3.96E−02 |
| | | transporter), member 5 | | |
| 269642 | Nat8l | N-acetyltransferase 8-like | 0.72 | 3.97E−02 |
| 21685 | Tef | thyrotroph embryonic factor | 0.72 | 4.05E−02 |
| 20970 | Sdc3 | syndecan 3 | 0.40 | 4.05E−02 |
| 70381 | Tecpr1 | tectonin beta-propeller repeat containing 1 | 0.34 | 4.10E−02 |
| 69852 | Tcf23 | transcription factor 23 | 0.69 | 4.21E−02 |
| 52710 | Slc52a2 | solute carrier protein 52, member 2 | 0.46 | 4.22E−02 |
| 12833 | Col6a1 | collagen, type VI, alpha 1 | 0.35 | 4.24E−02 |
| 245666 | Iqsec2 | IQ motif and Sec7 domain 2 | 0.41 | 4.24E−02 |
| 18208 | Ntn1 | netrin 1 | 0.37 | 4.30E−02 |
| 101240 | Wdr91 | WD repeat domain 91 | 0.64 | 4.30E−02 |
| 56741 | Igdcc4 | immunoglobulin superfamily, DCC subclass, | 0.39 | 4.32E−02 |
| | | member 4 | | |
| 233210 | Prr12 | proline rich 12 | 0.55 | 4.33E−02 |
| 207592 | Tbc1d16 | TBC1 domain family, member 16 | 0.37 | 4.41E−02 |
| 14462 | Gata3 | GATA binding protein 3 | 1.59 | 4.42E−02 |
| 19125 | Prodh | proline dehydrogenase | 0.57 | 4.43E−02 |
| 192201 | Wfdc15b | WAP four-disulfide core domain 15B | 0.01 | 4.43E−02 |
| 14182 | Fgfr1 | fibroblast growth factor receptor 1 | 0.39 | 4.43E−02 |
| 22151 | Tubb2a | tubulin, beta 2A class IIA | 0.36 | 4.43E−02 |
| 68171 | Pate6 | prostate and testis expressed 6 | 0.01 | 4.55E−02 |
| 22004 | Tpm2 | tropomyosin 2, beta | 0.30 | 4.57E−02 |
| 67131 | Acbd4 | acyl-Coenzyme A binding domain containing 4 | 0.41 | 4.57E−02 |
| 67916 | Plpp3 | phospholipid phosphatase 3 | 0.31 | 4.58E−02 |
| 15193 | Hdgfl2 | HDGF like 2 | 0.36 | 4.58E−02 |
| 17268 | Meis1 | Meis homeobox 1 | 0.33 | 4.60E−02 |
| 14245 | Lpin1 | lipin 1 | 0.43 | 4.66E−02 |
| 11732 | Ank | progressive ankylosis | 0.30 | 4.66E−02 |
| 66306 | Fam53c | family with sequence similarity 53, member C | 0.39 | 4.66E−02 |
| 12411 | Cbs | cystathionine beta-synthase | 0.33 | 4.75E−02 |
| 268739 | Arhgef40 | Rho guanine nucleotide exchange factor (GEF) | 0.36 | 4.75E−02 |
| | | 40 | | |
| 73225 | Fam118a | family with sequence similarity 118, member A | 0.38 | 4.75E−02 |
| 97775 | D930048N14Rik | RIKEN cDNA D930048N14 gene | 1.02 | 4.79E−02 |
| 269608 | Plekhg5 | pleckstrin homology domain containing, family G | 0.49 | 4.79E−02 |
| | | (with RhoGef domain) member 5 | | |
| 320129 | Grk3 | G protein-coupled receptor kinase 3 | 0.37 | 4.81E−02 |
| 67369 | Qpctl | glutaminyl-peptide cyclotransferase-like | 0.46 | 4.81E−02 |
| 18452 | P4ha2 | procollagen-proline, 2-oxoglutarate 4- | 0.45 | 4.85E−02 |
| | | dioxygenase (proline 4-hydroxylase), alpha II | | |
| | | polypeptide | | |
| 171531 | Mlph | melanophilin | 0.29 | 4.88E−02 |
| 226075 | Glis3 | GLIS family zinc finger 3 | 0.51 | 4.88E−02 |
| 12737 | Cldn1 | claudin 1 | 0.90 | 4.93E−02 |
| 11835 | Ar | androgen receptor | 1.20 | 4.97E−02 |

TABLE S3D
Downregulated genes in colon.

| | | | | FDR- |
|---|---|---|---|---|
| | | | | adjusted |
| Entrez | | | log2 fold- | Wald test |
| Gene ID | Symbol | Name | change | P-value |
| 21818 | Tgm3 | transglutaminase 3, E polypeptide | −1.21 | 9.45E−14 |
| 54613 | St3gal6 | ST3 beta-galactoside alpha-2,3-sialyltransferase | −0.82 | 1.06E−11 |
| | | 6 | | |
| 20531 | Slc34a2 | solute carrier family 34 (sodium phosphate), | −1.07 | 1.08E−09 |
| | | member 2 | | |
| 13830 | Stom | stomatin | −0.64 | 1.14E−09 |
| 216225 | Slc5a8 | solute carrier family 5 (iodide transporter), | −0.56 | 5.35E−09 |
| | | member 8 | | |
| 18484 | Pam | peptidylglycine alpha-amidating monooxygenase | −0.50 | 5.35E−09 |
| 11829 | Aqp4 | aquaporin 4 | −0.67 | 9.65E−09 |
| 17288 | Mep1b | meprin 1 beta | −0.57 | 9.66E−08 |
| 13195 | Ddc | dopa decarboxylase | −0.64 | 1.43E−07 |
| 17063 | Muc13 | mucin 13, epithelial transmembrane | −0.47 | 1.43E−07 |
| 100042514 | Sprr2a3 | small proline-rich protein 2A3 | −0.89 | 1.62E−07 |
| 70163 | Lypd8 | LY6/PLAUR domain containing 8 | −0.64 | 2.33E−07 |
| 12716 | Ckmt1 | creatine kinase, mitochondrial 1, ubiquitous | −0.62 | 3.76E−07 |
| 21952 | Tnni1 | troponin I, skeletal, slow 1 | −1.14 | 3.87E−07 |
| 235135 | Tmem45b | transmembrane protein 45b | −0.53 | 4.01E−07 |
| 230163 | Aldob | aldolase B, fructose-bisphosphate | −0.54 | 4.01E−07 |
| 433023 | Gm5485 | predicted gene 5485 | −0.73 | 9.25E−07 |
| 16173 | Il18 | interleukin 18 | −0.86 | 1.13E−06 |
| 667373 | Ifit1bl1 | interferon induced protein with tetratricpeptide | −0.82 | 1.19E−06 |
| | | repeats 1B like 1 | | |
| 13808 | Eno3 | enolase 3, beta muscle | −1.06 | 1.33E−06 |
| 23844 | Clca1 | chloride channel accessory 1 | −0.76 | 1.33E−06 |
| 224796 | Clic5 | chloride intracellular channel 5 | −0.46 | 1.40E−06 |
| 64381 | Ms4a8a | membrane-spanning 4-domains, subfamily A, | −0.51 | 1.44E−06 |
| | | member 8A | | |
| 11833 | Aqp8 | aquaporin 8 | −0.61 | 1.46E−06 |
| 14675 | Gna14 | guanine nucleotide binding protein, alpha 14 | −0.70 | 1.46E−06 |
| 100313519 | Snord17 | small nucleolar RNA, C/D box 17 | −0.67 | 2.72E−06 |
| 30962 | Slc7a9 | solute carrier family 7 (cationic amino acid | −1.03 | 3.07E−06 |
| | | transporter, y+ system), member 9 | | |
| 433247 | Cyp2c68 | cytochrome P450, family 2, subfamily c, | −0.86 | 3.69E−06 |
| | | polypeptide 68 | | |
| 66261 | Tm4sf20 | transmembrane 4 L six family member 20 | −0.54 | 5.63E−06 |
| 414084 | Tnip3 | TNFAIP3 interacting protein 3 | −1.18 | 7.77E−06 |
| 15212 | Hexb | hexosaminidase B | −0.82 | 8.05E−06 |
| 66857 | Plbd1 | phospholipase B domain containing 1 | −0.62 | 8.44E−06 |
| 21828 | Thbs4 | thrombospondin 4 | −0.62 | 1.04E−05 |
| 56362 | Sult1b1 | sulfotransferase family 1B, member 1 | −0.53 | 1.08E−05 |
| 71601 | Ceacam20 | carcinoembryonic antigen-related cell adhesion | −0.56 | 1.19E−05 |
| | | molecule 20 | | |
| 107747 | Aldh1l1 | aldehyde dehydrogenase 1 family, member L1 | −0.68 | 1.31E−05 |
| 56838 | Ccl28 | chemokine (C-C motif) ligand 28 | −0.55 | 1.69E−05 |
| 11745 | Anxa3 | annexin A3 | −1.00 | 1.76E−05 |
| 237038 | Nox1 | NADPH oxidase 1 | −0.91 | 1.86E−05 |
| 225997 | Trpm6 | transient receptor potential cation channel, | −0.72 | 2.01E−05 |
| | | subfamily M, member 6 | | |
| 70101 | Cyp4f16 | cytochrome P450, family 4, subfamily f, | −0.66 | 2.02E−05 |
| | | polypeptide 16 | | |
| 213819 | Casd1 | CAS1 domain containing 1 | −0.48 | 2.10E−05 |
| 56473 | Fads2 | fatty acid desaturase 2 | −0.51 | 2.86E−05 |
| 240638 | Slc16a12 | solute carrier family 16 (monocarboxylic acid | −0.94 | 2.92E−05 |
| | | transporters), member 12 | | |
| 16612 | Klk1 | kallikrein 1 | −0.44 | 3.12E−05 |
| 56643 | Slc15a1 | solute carrier family 15 (oligopeptide transporter), | −0.63 | 3.61E−05 |
| | | member 1 | | |
| 26357 | Abcg2 | ATP binding cassette subfamily G member 2 | −0.50 | 3.86E−05 |
| | | (Junior blood group) | | |
| 223631 | Ly6g2 | lymphocyte antigen 6 complex, locus G2 | −0.85 | 5.02E−05 |
| 12369 | Casp7 | caspase 7 | −0.38 | 5.14E−05 |
| 11865 | Arntl | aryl hydrocarbon receptor nuclear translocator- | −1.01 | 5.39E−05 |
| | | like | | |
| 14590 | Ggh | gamma-glutamyl hydrolase | −0.80 | 8.42E−05 |
| 18583 | Pde7a | phosphodiesterase 7A | −0.63 | 8.93E−05 |
| 625286 | Tmem236 | transmembrane protein 236 | −0.43 | 9.23E−05 |
| 102022 | Ces2a | carboxylesterase 2A | −0.77 | 9.33E−05 |
| 244853 | Nxpe4 | neurexophilin and PC-esterase domain family, | −0.70 | 1.09E−04 |
| | | member 4 | | |
| 56434 | Tspan3 | tetraspanin 3 | −0.37 | 1.15E−04 |
| 74559 | Elovl7 | ELOVL family member 7, elongation of long chain | −0.40 | 1.15E−04 |
| | | fatty acids (yeast) | | |
| 381122 | Capn13 | calpain 13 | −0.78 | 1.16E−04 |
| 53945 | Slc40a1 | solute carrier family 40 (iron-regulated | −0.69 | 1.17E−04 |
| | | transporter), member 1 | | |
| 667865 | Igkv6-17 | immunoglobulin kappa variable 6-17 | −2.05 | 1.21E−04 |
| 209378 | Itih5 | inter-alpha (globulin) inhibitor H5 | −0.43 | 1.21E−04 |
| 20363 | Selenop | selenoprotein P | −0.57 | 1.21E−04 |
| 50877 | Neu3 | neuraminidase 3 | −1.04 | 1.21E−04 |
| 21898 | Tlr4 | toll-like receptor 4 | −0.58 | 1.34E−04 |
| 107569 | Nt5c3 | 5′-nucleotidase, cytosolic III | −0.62 | 1.34E−04 |
| 19064 | Ppy | pancreatic polypeptide | −1.36 | 1.40E−04 |
| 11522 | Adh1 | alcohol dehydrogenase 1 (class I) | −0.77 | 1.54E−04 |
| 69693 | Pof1b | premature ovarian failure 1B | −0.43 | 1.67E−04 |
| 66805 | Tspan1 | tetraspanin 1 | −0.41 | 1.89E−04 |
| 67701 | Wfdc2 | WAP four-disulfide core domain 2 | −0.85 | 1.92E−04 |
| 170706 | Tmem37 | transmembrane protein 37 | −0.93 | 1.94E−04 |
| 108907 | Nusap1 | nucleolar and spindle associated protein 1 | −0.49 | 2.31E−04 |
| 16425 | Itih2 | inter-alpha trypsin inhibitor, heavy chain 2 | −0.84 | 2.52E−04 |
| 67603 | Dusp6 | dual specificity phosphatase 6 | −0.64 | 2.69E−04 |
| 93961 | B3galt5 | UDP-Gal:betaGlcNAc beta 1,3- | −0.40 | 2.76E−04 |
| | | galactosyltransferase, polypeptide 5 | | |
| 71578 | Sval1 | seminal vesicle antigen-like 1 | −0.90 | 2.81E−04 |
| 101118 | Tmem168 | transmembrane protein 168 | −0.48 | 2.99E−04 |
| 74548 | Gsdmc4 | gasdermin C4 | −0.66 | 3.88E−04 |
| 217212 | Pyy | peptide YY | −0.64 | 4.13E−04 |
| 18703 | Pigr | polymeric immunoglobulin receptor | −0.44 | 4.32E−04 |
| 108005 | Igkv3-7 | immunoglobulin kappa variable 3-7 | −0.12 | 4.37E−04 |
| 17940 | Naip1 | NLR family, apoptosis inhibitory protein 1 | −0.44 | 5.24E−04 |
| 12012 | Baat | bile acid-Coenzyme A: amino acid N- | −1.69 | 5.56E−04 |
| | | acyltransferase | | |
| 13101 | Cyp2d10 | cytochrome P450, family 2, subfamily d, | −0.70 | 6.01E−04 |
| | | polypeptide 10 | | |
| 319157 | H4c6 | H4 clustered histone 6 | −0.56 | 6.01E−04 |
| 241770 | Rims4 | regulating synaptic membrane exocytosis 4 | −0.82 | 6.13E−04 |
| 26944 | Tinag | tubulointerstitial nephritis antigen | −0.53 | 6.23E−04 |
| 234515 | Inpp4b | inositol polyphosphate-4-phosphatase, type II | −0.52 | 6.26E−04 |
| 56185 | Hao2 | hydroxyacid oxidase 2 | −0.52 | 6.72E−04 |
| 110454 | Ly6a | lymphocyte antigen 6 complex, locus A | −0.72 | 7.41E−04 |
| 209086 | Samd9l | sterile alpha motif domain containing 9-like | −0.34 | 7.67E−04 |
| 67758 | Aadac | arylacetamide deacetylase | −0.51 | 7.81E−04 |
| 107770 | Tm6sf2 | transmembrane 6 superfamily member 2 | −0.61 | 7.87E−04 |
| 101437 | Dhx32 | DEAH (Asp-Glu-Ala-His) box polypeptide 32 | −0.33 | 8.12E−04 |
| 433470 | AA467197 | expressed sequence AA467197 | −0.50 | 8.90E−04 |
| 13034 | Ctse | cathepsin E | −0.85 | 9.55E−04 |
| 66569 | Gdpd1 | glycerophosphodiester phosphodiesterase | −0.35 | 1.02E−03 |
| | | domain containing 1 | | |
| 22042 | Tfrc | transferrin receptor | −0.40 | 1.05E−03 |
| 229782 | Slc35a3 | solute carrier family 35 (UDP-N- | −0.35 | 1.13E−03 |
| | | acetylglucosamine (UDP-GlcNAc) transporter), | | |
| | | member 3 | | |
| 80898 | Erap1 | endoplasmic reticulum aminopeptidase 1 | −0.34 | 1.21E−03 |
| 105348 | Golm1 | golgi membrane protein 1 | −0.32 | 1.21E−03 |
| 15519 | Hsp90aa1 | heat shock protein 90, alpha (cytosolic), class A | −0.38 | 1.23E−03 |
| | | member 1 | | |
| 17311 | Kitl | kit ligand | −0.36 | 1.23E−03 |
| 14282 | Fosb | FBJ osteosarcoma oncogene B | −1.42 | 1.28E−03 |
| 109901 | Cela1 | chymotrypsin-like elastase family, member 1 | −0.68 | 1.29E−03 |
| 12753 | Clock | circadian locomotor output cycles kaput | −0.35 | 1.34E−03 |
| 269181 | Mgat4a | mannoside acetylglucosaminyltransferase 4, | −0.46 | 1.45E−03 |
| | | isoenzyme A | | |
| 23919 | Insl5 | insulin-like 5 | −0.71 | 1.49E−03 |
| 58185 | Rsad2 | radical S-adenosyl methionine domain containing | −0.75 | 1.50E−03 |
| | | 2 | | |
| 20773 | Sptlc2 | serine palmitoyltransferase, long chain base | −0.30 | 1.67E−03 |
| | | subunit 2 | | |
| 54392 | Ncapg | non-SMC condensin I complex, subunit G | −0.60 | 1.73E−03 |
| 108105 | B3gnt5 | UDP-GlcNAc:betaGal beta-1,3-N- | −0.62 | 1.76E−03 |
| | | acetylglucosaminyltransferase 5 | | |
| 381213 | Ms4a12 | membrane-spanning 4-domains, subfamily A, | −0.62 | 1.76E−03 |
| | | member 12 | | |
| 15481 | Hspa8 | heat shock protein 8 | −0.32 | 1.77E−03 |
| 20216 | Acsm3 | acyl-CoA synthetase medium-chain family | −0.55 | 1.80E−03 |
| | | member 3 | | |
| 115485773 | Gm25395 | predicted gene, 25395 | −0.77 | 1.94E−03 |
| 56774 | Slc6a14 | solute carrier family 6 (neurotransmitter | −0.32 | 2.16E−03 |
| | | transporter), member 14 | | |
| 12902 | Cr2 | complement receptor 2 | −0.79 | 2.32E−03 |
| 432555 | Gm5431 | predicted gene 5431 | −1.03 | 2.34E−03 |
| 16774 | Lama3 | laminin, alpha 3 | −0.38 | 2.44E−03 |
| 14873 | Gsto1 | glutathione S-transferase omega 1 | −0.31 | 2.44E−03 |
| 239606 | Slc2a13 | solute carrier family 2 (facilitated glucose | −0.44 | 2.49E−03 |
| | | transporter), member 13 | | |
| 331063 | Gsdmc2 | gasdermin C2 | −0.47 | 2.53E−03 |
| 16571 | Kif4 | kinesin family member 4 | −0.54 | 2.61E−03 |
| 270328 | Gsdmc3 | gasdermin C3 | −0.80 | 2.61E−03 |
| 268301 | Sowahc | sosondowah ankyrin repeat domain family | −0.42 | 2.67E−03 |
| | | member C | | |
| 231999 | Plekha8 | pleckstrin homology domain containing, family A | −0.32 | 2.83E−03 |
| | | (phosphoinositide binding specific) member 8 | | |
| 67092 | Gatm | glycine amidinotransferase (L-arginine:glycine | −0.68 | 2.83E−03 |
| | | amidinotransferase) | | |
| 20510 | Slc1a1 | solute carrier family 1 (neuronal/epithelial high | −0.59 | 2.83E−03 |
| | | affinity glutamate transporter, system Xag), | | |
| | | member 1 | | |
| 11740 | Slc25a5 | solute carrier family 25 (mitochondrial carrier, | −0.25 | 2.92E−03 |
| | | adenine nucleotide translocator), member 5 | | |
| 53881 | Slc5a3 | solute carrier family 5 (inositol transporters), | −0.45 | 3.34E−03 |
| | | member 3 | | |
| 319150 | H3c2 | H3 clustered histone 2 | −0.50 | 3.46E−03 |
| 100434 | Slc44a1 | solute carrier family 44, member 1 | −0.34 | 3.57E−03 |
| 14211 | Smc2 | structural maintenance of chromosomes 2 | −0.44 | 3.83E−03 |
| 216820 | Dhrs7b | dehydrogenase/reductase (SDR family) member | −0.35 | 3.88E−03 |
| | | 7B | | |
| 13040 | Ctss | cathepsin S | −0.40 | 3.91E−03 |
| 14869 | Gstp2 | glutathione S-transferase, pi 2 | −0.58 | 3.93E−03 |
| 244416 | Ppp1r3b | protein phosphatase 1, regulatory subunit 3B | −0.53 | 3.98E−03 |
| 100503565 | Gm9926 | predicted gene 9926 | −0.48 | 3.99E−03 |
| 94192 | C1galt1 | core 1 synthase, glycoprotein-N- | −0.27 | 4.07E−03 |
| | | acetylgalactosamine 3-beta- | | |
| | | galactosyltransferase, 1 | | |
| 23962 | Oasl2 | 2′-5′ oligoadenylate synthetase-like 2 | −0.66 | 4.18E−03 |
| 213570 | Ighv1-78 | immunoglobulin heavy variable 1-78 | −0.11 | 4.18E−03 |
| 65972 | Ifi30 | interferon gamma inducible protein 30 | −0.28 | 4.40E−03 |
| 319149 | H3c4 | H3 clustered histone 4 | −0.69 | 4.42E−03 |
| 12475 | Cd14 | CD14 antigen | −0.44 | 4.42E−03 |
| 71701 | Pnpt1 | polyribonucleotide nucleotidyltransferase 1 | −0.39 | 4.57E−03 |
| 50709 | H1f4 | H1.4 linker histone, cluster member | −0.36 | 4.63E−03 |
| 50708 | H1f2 | H1.2 linker histone, cluster member | −0.32 | 4.63E−03 |
| 114128 | Laptm4b | lysosomal-associated protein transmembrane 4B | −0.36 | 4.76E−03 |
| 17708 | COX1 | cytochrome c oxidase subunit I | −0.41 | 4.80E−03 |
| 239853 | Adgrg7 | adhesion G protein-coupled receptor G7 | −0.34 | 4.80E−03 |
| 777648 | Ighj1 | immunoglobulin heavy joining 1 | −1.59 | 4.88E−03 |
| 67121 | Mastl | microtubule associated serine/threonine kinase- | −0.53 | 5.08E−03 |
| | | like | | |
| 20482 | Skil | SKI-like | −0.39 | 5.38E−03 |
| 19683 | Rdh16 | retinol dehydrogenase 16 | −0.68 | 5.42E−03 |
| 12579 | Cdkn2b | cyclin dependent kinase inhibitor 2B | −0.50 | 5.69E−03 |
| 14957 | H1f3 | H1.3 linker histone, cluster member | −0.45 | 5.76E−03 |
| 78789 | Vsig1 | V-set and immunoglobulin domain containing 1 | −1.14 | 6.49E−03 |
| 56421 | Pfkp | phosphofructokinase, platelet | −0.28 | 6.49E−03 |
| 229927 | Clca3b | chloride channel accessory 3B | −0.37 | 6.76E−03 |
| 17279 | Melk | maternal embryonic leucine zipper kinase | −0.41 | 6.84E−03 |
| 243385 | Gprin3 | GPRIN family member 3 | −0.38 | 7.04E−03 |
| 319186 | H2bc14 | H2B clustered histone 14 | −0.69 | 7.04E−03 |
| 78653 | Bola3 | bolA-like 3 (E. coli) | −0.44 | 7.04E−03 |
| 56318 | Acpp | acid phosphatase, prostate | −0.40 | 7.04E−03 |
| 223706 | Cyp2d34 | cytochrome P450, family 2, subfamily d, | −0.36 | 7.04E−03 |
| | | polypeptide 34 | | |
| 70045 | 2610528A11Rik | RIKEN cDNA 2610528A11 gene | −0.48 | 7.04E−03 |
| 14425 | Galnt3 | polypeptide N-acetylgalactosaminyltransferase 3 | −0.36 | 7.08E−03 |
| 73910 | Arhgap18 | Rho GTPase activating protein 18 | −0.28 | 7.08E−03 |
| 97122 | H4c14 | H4 clustered histone 14 | −0.43 | 7.35E−03 |
| 67664 | Rnf125 | ring finger protein 125 | −0.73 | 7.63E−03 |
| 12269 | C4bp | complement component 4 binding protein | −0.74 | 7.65E−03 |
| 12858 | Cox5a | cytochrome c oxidase subunit 5A | −0.35 | 7.75E−03 |
| 70261 | Chp2 | calcineurin-like EF hand protein 2 | −0.43 | 7.75E−03 |
| 14423 | Galnt1 | polypeptide N-acetylgalactosaminyltransferase 1 | −0.29 | 7.84E−03 |
| 271844 | Pla2g4f | phospholipase A2, group IVF | −0.87 | 7.97E−03 |
| 218977 | Dlgap5 | DLG associated protein 5 | −0.46 | 8.32E−03 |
| 71907 | Serpina9 | serine (or cysteine) peptidase inhibitor, clade A | −1.18 | 8.32E−03 |
| | | (alpha-1 antiproteinase, antitrypsin), member 9 | | |
| 226971 | Plekhb2 | pleckstrin homology domain containing, family B | −0.31 | 8.42E−03 |
| | | (evectins) member 2 | | |
| 18968 | Pola1 | polymerase (DNA directed), alpha 1 | −0.55 | 8.42E−03 |
| 26565 | Pla2g10 | phospholipase A2, group X | −0.47 | 8.43E−03 |
| 17076 | Ly75 | lymphocyte antigen 75 | −0.46 | 8.43E−03 |
| 17075 | Epcam | epithelial cell adhesion molecule | −0.25 | 8.64E−03 |
| 15957 | Ifit1 | interferon-induced protein with tetratricopeptide | −0.65 | 8.65E−03 |
| | | repeats 1 | | |
| 100061 | Lrrc19 | leucine rich repeat containing 19 | −0.42 | 8.65E−03 |
| 115487534 | Gm24119 | predicted gene, 24119 | −1.43 | 9.11E−03 |
| 21353 | Tank | TRAF family member-associated Nf-kappa B | −0.46 | 9.67E−03 |
| | | activator | | |
| 20133 | Rrm1 | ribonucleotide reductase M1 | −0.42 | 9.83E−03 |
| 14537 | Gcnt1 | glucosaminyl (N-acetyl) transferase 1, core 2 | −0.47 | 9.93E−03 |
| 212285 | Arap2 | ArfGAP with RhoGAP domain, ankyrin repeat and | −0.29 | 1.02E−02 |
| | | PH domain 2 | | |
| 22785 | Slc30a4 | solute carrier family 30 (zinc transporter), member | −0.26 | 1.02E−02 |
| | | 4 | | |
| 18950 | Pnp | purine-nucleoside phosphorylase | −0.41 | 1.04E−02 |
| 100036521 | Umad1 | UMAP1-MVP12 associated (UMA) domain | −0.44 | 1.06E−02 |
| | | containing 1 | | |
| 22041 | Trf | transferrin | −0.35 | 1.06E−02 |
| 97212 | Hadha | hydroxyacyl-CoA dehydrogenase trifunctional | −0.30 | 1.06E−02 |
| | | multienzyme complex subunit alpha | | |
| 17219 | Mcm6 | minichromosome maintenance complex | −0.44 | 1.08E−02 |
| | | component 6 | | |
| 12419 | Cbx5 | chromobox 5 | −0.26 | 1.10E−02 |
| 98685 | Trmt1l | tRNA methyltransferase 1 like | −0.43 | 1.10E−02 |
| 99712 | Cept1 | choline/ethanolaminephosphotransferase 1 | −0.36 | 1.10E−02 |
| 12362 | Casp1 | caspase 1 | −0.35 | 1.11E−02 |
| 268860 | Abat | 4-aminobutyrate aminotransferase | −0.36 | 1.11E−02 |
| 21946 | Pglyrp1 | peptidoglycan recognition protein 1 | −0.45 | 1.12E−02 |
| 54357 | Epb41l4b | erythrocyte membrane protein band 4.1 like 4b | −0.25 | 1.14E−02 |
| 27273 | Pdk4 | pyruvate dehydrogenase kinase, isoenzyme 4 | −0.81 | 1.15E−02 |
| 107515 | Lgr4 | leucine-rich repeat-containing G protein-coupled | −0.29 | 1.16E−02 |
| | | receptor 4 | | |
| 52466 | Slc46a1 | solute carrier family 46, member 1 | −0.65 | 1.16E−02 |
| 83379 | Klb | klotho beta | −0.57 | 1.17E−02 |
| 24071 | Synj2bp | synaptojanin 2 binding protein | −0.39 | 1.18E−02 |
| 381778 | Igkv16-104 | immunoglobulin kappa variable 16-104 | −0.04 | 1.21E−02 |
| 66917 | Chordc1 | cysteine and histidine-rich domain (CHORD)- | −0.39 | 1.21E−02 |
| | | containing, zinc-binding protein 1 | | |
| 66270 | Retreg1 | reticulophagy regulator 1 | −0.40 | 1.21E−02 |
| 12453 | Ccni | cyclin I | −0.28 | 1.21E−02 |
| 212943 | Tent5a | terminal nucleotidyltransferase 5A | −0.43 | 1.23E−02 |
| 225742 | St8sia5 | ST8 alpha-N-acetyl-neuraminide alpha-2,8- | −1.14 | 1.25E−02 |
| | | sialyltransferase 5 | | |
| 212070 | Clrn3 | clarin 3 | −0.26 | 1.28E−02 |
| 21786 | Tff3 | trefoil factor 3, intestinal | −0.39 | 1.32E−02 |
| 15211 | Hexa | hexosaminidase A | −0.26 | 1.36E−02 |
| 15203 | Heph | hephaestin | −0.27 | 1.36E−02 |
| 21973 | Top2a | topoisomerase (DNA) II alpha | −0.42 | 1.36E−02 |
| 19775 | Xpr1 | xenotropic and polytropic retrovirus receptor 1 | −0.33 | 1.36E−02 |
| 22682 | Zfand5 | zinc finger, AN1-type domain 5 | −0.34 | 1.37E−02 |
| 102632 | Acad11 | acyl-Coenzyme A dehydrogenase family, member | −0.29 | 1.37E−02 |
| | | 11 | | |
| 70472 | Atad2 | ATPase family, AAA domain containing 2 | −0.38 | 1.40E−02 |
| 27409 | Abcg5 | ATP binding cassette subfamily G member 5 | −0.77 | 1.40E−02 |
| 667683 | Igkv4-59 | immunoglobulin kappa variable 4-59 | −1.18 | 1.41E−02 |
| 67276 | Eri1 | exoribonuclease 1 | −0.33 | 1.43E−02 |
| 76464 | Knl1 | kinetochore scaffold 1 | −0.42 | 1.43E−02 |
| 108670 | Epsti1 | epithelial stromal interaction 1 (breast) | −0.33 | 1.43E−02 |
| 227327 | B3gnt7 | UDP-GlcNAc:betaGal beta-1,3-N- | −0.59 | 1.45E−02 |
| | | acetylglucosaminyltransferase 7 | | |
| 14469 | Gbp2 | guanylate binding protein 2 | −0.58 | 1.47E−02 |
| 226564 | Fmo4 | flavin containing monooxygenase 4 | −0.63 | 1.47E−02 |
| 12557 | Cdh17 | cadherin 17 | −0.29 | 1.47E−02 |
| 74117 | Actr3 | ARP3 actin-related protein 3 | −0.22 | 1.48E−02 |
| 81489 | Dnajb1 | DnaJ heat shock protein family (Hsp40) member | −0.37 | 1.49E−02 |
| | | B1 | | |
| 12387 | Ctnnb1 | catenin (cadherin associated protein), beta 1 | −0.23 | 1.50E−02 |
| 237911 | Brip1 | BRCA1 interacting protein C-terminal helicase 1 | −0.50 | 1.50E−02 |
| 66841 | Etfdh | electron transferring flavoprotein, dehydrogenase | −0.26 | 1.50E−02 |
| 23959 | Nt5e | 5′ nucleotidase, ecto | −0.52 | 1.52E−02 |
| 99526 | Usp53 | ubiquitin specific peptidase 53 | −0.34 | 1.56E−02 |
| 27061 | Bcap31 | B cell receptor associated protein 31 | −0.27 | 1.58E−02 |
| 72900 | Ndufv2 | NADH:ubiquinone oxidoreductase core subunit | −0.30 | 1.66E−02 |
| | | V2 | | |
| 243659 | Styk1 | serine/threonine/tyrosine kinase 1 | −0.33 | 1.67E−02 |
| 619846 | Igkv5-48 | immunoglobulin kappa variable 5-48 | −0.03 | 1.75E−02 |
| 13370 | Dio1 | deiodinase, iodothyronine, type I | −0.42 | 1.75E−02 |
| 54381 | Cpq | carboxypeptidase Q | −0.33 | 1.75E−02 |
| 70693 | Adgra3 | adhesion G protein-coupled receptor A3 | −0.25 | 1.75E−02 |
| 105171 | Arrdc3 | arrestin domain containing 3 | −0.38 | 1.81E−02 |
| 105892 | 9030619P08Rik | lymphocyte antigen 6 complex pseudogene | −0.13 | 1.82E−02 |
| 12540 | Cdc42 | cell division cycle 42 | −0.26 | 1.85E−02 |
| 57319 | Smpdl3a | sphingomyelin phosphodiesterase, acid-like 3A | −0.42 | 1.90E−02 |
| 75415 | Arhgap12 | Rho GTPase activating protein 12 | −0.28 | 1.90E−02 |
| 68421 | Lmbrd1 | LMBR1 domain containing 1 | −0.25 | 1.93E−02 |
| 545370 | Hmcn1 | hemicentin 1 | −0.39 | 2.03E−02 |
| 243439 | Igkv14-100 | immunoglobulin kappa chain variable 14-100 | −0.05 | 2.05E−02 |
| 29811 | Ndrg2 | N-myc downstream regulated gene 2 | −0.33 | 2.08E−02 |
| 21897 | Tlr1 | toll-like receptor 1 | −0.34 | 2.09E−02 |
| 58998 | Nectin3 | nectin cell adhesion molecule 3 | −0.25 | 2.12E−02 |
| 73804 | Kif2c | kinesin family member 2C | −0.47 | 2.12E−02 |
| 319181 | H2bc8 | H2B clustered histone 8 | −0.45 | 2.14E−02 |
| 319848 | Slc17a4 | solute carrier family 17 (sodium phosphate), | −0.39 | 2.16E−02 |
| | | member 4 | | |
| 17329 | Cxcl9 | chemokine (C-X-C motif) ligand 9 | −0.14 | 2.16E−02 |
| 107350 | AW112010 | expressed sequence AW112010 | −0.52 | 2.16E−02 |
| 57279 | Slc25a20 | solute carrier family 25 (mitochondrial | −0.31 | 2.18E−02 |
| | | carnitine/acylcarnitine translocase), member 20 | | |
| 109731 | Maob | monoamine oxidase B | −0.38 | 2.23E−02 |
| 76740 | Efr3a | EFR3 homolog A | −0.26 | 2.28E−02 |
| 98386 | Lbr | lamin B receptor | −0.29 | 2.29E−02 |
| 55985 | Cxcl13 | chemokine (C-X-C motif) ligand 13 | −0.49 | 2.33E−02 |
| 692169 | Igkv15-103 | immunoglobulin kappa chain variable 15-103 | −0.07 | 2.33E−02 |
| 27392 | Pign | phosphatidylinositol glycan anchor biosynthesis, | −0.29 | 2.34E−02 |
| | | class N | | |
| 246730 | Oas1a | 2′-5′ oligoadenylate synthetase 1A | −0.42 | 2.34E−02 |
| 212862 | Chpt1 | choline phosphotransferase 1 | −0.27 | 2.34E−02 |
| 18003 | Nedd9 | neural precursor cell expressed, developmentally | −0.33 | 2.35E−02 |
| | | down-regulated gene 9 | | |
| 626848 | Zfp971 | zinc finger protein 971 | −0.46 | 2.37E−02 |
| 360198 | H3c1 | H3 clustered histone 1 | −0.50 | 2.45E−02 |
| 72787 | Ndc1 | NDC1 transmembrane nucleoporin | −0.42 | 2.46E−02 |
| 101148 | Bmt2 | base methyltransferase of 25S rRNA 2 | −0.38 | 2.46E−02 |
| 67171 | Dram2 | DNA-damage regulated autophagy modulator 2 | −0.27 | 2.46E−02 |
| 241639 | Fermt1 | fermitin family member 1 | −0.30 | 2.46E−02 |
| 20687 | Sp3 | trans-acting transcription factor 3 | −0.25 | 2.46E−02 |
| 22171 | Tyms | thymidylate synthase | −0.45 | 2.46E−02 |
| 71586 | Ifih1 | interferon induced with helicase C domain 1 | −0.29 | 2.46E−02 |
| 16420 | Itgb6 | integrin beta 6 | −0.29 | 2.49E−02 |
| 100043108 | Cyp2c69 | cytochrome P450, family 2, subfamily c, | −0.56 | 2.49E−02 |
| | | polypeptide 69 | | |
| 78937 | Avl9 | AVL9 cell migration associated | −0.29 | 2.49E−02 |
| 12235 | Bub1 | BUB1, mitotic checkpoint serine/threonine kinase | −0.51 | 2.49E−02 |
| 193740 | Hspa1a | heat shock protein 1A | −0.75 | 2.52E−02 |
| 56348 | Hsd17b12 | hydroxysteroid (17-beta) dehydrogenase 12 | −0.33 | 2.53E−02 |
| 67270 | Mrpl42 | mitochondrial ribosomal protein L42 | −0.30 | 2.53E−02 |
| 16647 | Kpna2 | karyopherin (importin) alpha 2 | −0.30 | 2.53E−02 |
| 18701 | Pigf | phosphatidylinositol glycan anchor biosynthesis, | −0.48 | 2.57E−02 |
| | | class F | | |
| 68262 | Agpat4 | 1-acylglycerol-3-phosphate O-acyltransferase 4 | −0.32 | 2.58E−02 |
| | | (lysophosphatidic acid acyltransferase, delta) | | |
| 94184 | Pdxdc1 | pyridoxal-dependent decarboxylase domain | −0.25 | 2.58E−02 |
| | | containing 1 | | |
| 117167 | Steap4 | STEAP family member 4 | −0.63 | 2.58E−02 |
| 12952 | Cry1 | cryptochrome 1 (photolyase-like) | −0.46 | 2.66E−02 |
| 66101 | Ppih | peptidyl prolyl isomerase H | −0.46 | 2.66E−02 |
| 110052 | Dek | DEK proto-oncogene (DNA binding) | −0.26 | 2.68E−02 |
| 240283 | Dmxl1 | Dmx-like 1 | −0.25 | 2.68E−02 |
| 382053 | Ces3a | carboxylesterase 3A | −1.12 | 2.68E−02 |
| 18242 | Oat | ornithine aminotransferase | −0.31 | 2.77E−02 |
| 76722 | Ckmt2 | creatine kinase, mitochondrial 2 | −0.74 | 2.83E−02 |
| 17156 | Man1a2 | mannosidase, alpha, class 1A, member 2 | −0.22 | 2.84E−02 |
| 67273 | Ndufa10 | NADH:ubiquinone oxidoreductase subunit A10 | −0.26 | 2.90E−02 |
| 28030 | Gfm1 | G elongation factor, mitochondrial 1 | −0.30 | 2.93E−02 |
| 70387 | Ttc9c | tetratricopeptide repeat domain 9C | −0.31 | 2.94E−02 |
| 434025 | Igkv9-120 | immunoglobulin kappa chain variable 9-120 | −0.05 | 2.99E−02 |
| 17218 | Mcm5 | minichromosome maintenance complex | −0.38 | 3.03E−02 |
| | | component 5 | | |
| 23960 | Oas1g | 2′-5′ oligoadenylate synthetase 1G | −0.48 | 3.03E−02 |
| 637515 | Nlrp1b | NLR family, pyrin domain containing 1B | −0.32 | 3.04E−02 |
| 56431 | Dstn | destrin | −0.26 | 3.06E−02 |
| 271377 | Zbtb11 | zinc finger and BTB domain containing 11 | −0.33 | 3.10E−02 |
| 15945 | Cxcl10 | chemokine (C-X-C motif) ligand 10 | −0.13 | 3.10E−02 |
| 16551 | Kif11 | kinesin family member 11 | −0.37 | 3.11E−02 |
| 18477 | Prdx1 | peroxiredoxin 1 | −0.26 | 3.11E−02 |
| 52440 | Tax1bp1 | Tax1 (human T cell leukemia virus type I) binding | −0.24 | 3.11E−02 |
| | | protein 1 | | |
| 219033 | Ang4 | angiogenin, ribonuclease A family, member 4 | −0.54 | 3.14E−02 |
| 67453 | Slc25a46 | solute carrier family 25, member 46 | −0.29 | 3.16E−02 |
| 71844 | Nupl1 | nucleoporin like 1 | −0.30 | 3.17E−02 |
| 74355 | Smchd1 | SMC hinge domain containing 1 | −0.27 | 3.19E−02 |
| 70337 | Iyd | iodotyrosine deiodinase | −0.45 | 3.21E−02 |
| 51885 | Tubgcp4 | tubulin, gamma complex associated protein 4 | −0.36 | 3.26E−02 |
| 66532 | Rep15 | RAB15 effector protein | −0.41 | 3.27E−02 |
| 107373 | Fam111a | family with sequence similarity 111, member A | −0.47 | 3.28E−02 |
| 320806 | Gfm2 | G elongation factor, mitochondrial 2 | −0.26 | 3.28E−02 |
| 12053 | Bcl6 | B cell leukemia/lymphoma 6 | −0.41 | 3.28E−02 |
| 244049 | Mctp2 | multiple C2 domains, transmembrane 2 | −0.31 | 3.28E−02 |
| 11783 | Apaf1 | apoptotic peptidase activating factor 1 | −0.32 | 3.29E−02 |
| 20443 | St3gal4 | ST3 beta-galactoside alpha-2,3-sialyltransferase | −0.34 | 3.29E−02 |
| | | 4 | | |
| 68743 | Anln | anillin, actin binding protein | −0.33 | 3.31E−02 |
| 631797 | Fer1l6 | fer-1-like 6 (C. elegans) | −0.31 | 3.31E−02 |
| 23991 | Cib1 | calcium and integrin binding 1 (calmyrin) | −0.26 | 3.31E−02 |
| 231296 | Lrrc66 | leucine rich repeat containing 66 | −0.35 | 3.31E−02 |
| 15078 | H3f3a | H3.3 histone A | −0.26 | 3.31E−02 |
| 110639 | Prps2 | phosphoribosyl pyrophosphate synthetase 2 | −0.31 | 3.31E−02 |
| 20409 | Ostf1 | osteoclast stimulating factor 1 | −0.26 | 3.35E−02 |
| 108150 | Galnt7 | polypeptide N-acetylgalactosaminyltransferase 7 | −0.25 | 3.35E−02 |
| 216578 | Papolg | poly(A) polymerase gamma | −0.38 | 3.38E−02 |
| 59030 | Mkks | McKusick-Kaufman syndrome | −0.45 | 3.41E−02 |
| 11520 | Plin2 | perilipin 2 | −0.30 | 3.50E−02 |
| 80838 | H1f1 | H1.1 linker histone, cluster member | −0.38 | 3.50E−02 |
| 15374 | Jpt1 | Jupiter microtubule associated homolog 1 | −0.32 | 3.52E−02 |
| 66882 | Bzw1 | basic leucine zipper and W2 domains 1 | −0.24 | 3.52E−02 |
| 15567 | Slc6a4 | solute carrier family 6 (neurotransmitter | −0.47 | 3.54E−02 |
| | | transporter, serotonin), member 4 | | |
| 268697 | Ccnb1 | cyclin B1 | −0.38 | 3.55E−02 |
| 215387 | Ncaph | non-SMC condensin I complex, subunit H | −0.35 | 3.56E−02 |
| 268297 | Scml4 | Scm polycomb group protein like 4 | −0.30 | 3.56E−02 |
| 71679 | Atp5h | ATP synthase, H+ transporting, mitochondrial F0 | −0.27 | 3.61E−02 |
| | | complex, subunit D | | |
| 76273 | Ndfip2 | Nedd4 family interacting protein 2 | −0.26 | 3.65E−02 |
| 66412 | Arrdc4 | arrestin domain containing 4 | −0.37 | 3.66E−02 |
| 12764 | Cmas | cytidine monophospho-N-acetylneuraminic acid | −0.27 | 3.67E−02 |
| | | synthetase | | |
| 74153 | Uba7 | ubiquitin-like modifier activating enzyme 7 | −0.31 | 3.70E−02 |
| 235339 | Dlat | dihydrolipoamide S-acetyltransferase (E2 | −0.24 | 3.70E−02 |
| | | component of pyruvate dehydrogenase complex) | | |
| 68059 | Tm9sf2 | transmembrane 9 superfamily member 2 | −0.20 | 3.72E−02 |
| 14663 | Glycam1 | glycosylation dependent cell adhesion molecule 1 | −0.11 | 3.76E−02 |
| 50798 | Gne | glucosamine (UDP-N-acetyl)-2-epimerase/N- | −0.26 | 3.78E−02 |
| | | acetylmannosamine kinase | | |
| 381306 | BC055324 | cDNA sequence BC055324 | −0.59 | 3.78E−02 |
| 227399 | Ppip5k2 | diphosphoinositol pentakisphosphate kinase 2 | −0.23 | 3.78E−02 |
| 242505 | Rasef | RAS and EF hand domain containing | −0.30 | 3.79E−02 |
| 75600 | Calml4 | calmodulin-like 4 | −0.27 | 3.79E−02 |
| 66075 | Chchd3 | coiled-coil-helix-coiled-coil-helix domain | −0.26 | 3.80E−02 |
| | | containing 3 | | |
| 115487256 | Gm26448 | predicted gene, 26448 | −0.53 | 3.83E−02 |
| 20419 | Shcbp1 | Sho SH2-domain binding protein 1 | −0.45 | 3.92E−02 |
| 70561 | Txndc16 | thioredoxin domain containing 16 | −0.28 | 3.93E−02 |
| 214523 | Tmprss4 | transmembrane protease, serine 4 | −0.26 | 3.93E−02 |
| 17345 | Mki67 | antigen identified by monoclonal antibody Ki 67 | −0.34 | 3.96E−02 |
| 108062 | Cstf2 | cleavage stimulation factor, 3′ pre-RNA subunit 2 | −0.26 | 3.96E−02 |
| 117198 | Ivns1abp | influenza virus NS1A binding protein | −0.26 | 4.05E−02 |
| 628571 | Igkv4-63 | immunoglobulin kappa variable 4-63 | −0.04 | 4.05E−02 |
| 68014 | Zwilch | zwilch kinetochore protein | −0.52 | 4.05E−02 |
| 67150 | Rnf141 | ring finger protein 141 | −0.24 | 4.06E−02 |
| 54484 | Mkrn1 | makorin, ring finger protein, 1 | −0.24 | 4.13E−02 |
| 76507 | Aoc1 | amine oxidase, copper-containing 1 | −0.40 | 4.18E−02 |
| 14674 | Gna13 | guanine nucleotide binding protein, alpha 13 | −0.25 | 4.22E−02 |
| 68026 | Pclaf | PCNA clamp associated factor | −0.38 | 4.26E−02 |
| 110750 | Cse1l | chromosome segregation 1-like (S. cerevisiae) | −0.25 | 4.32E−02 |
| 99382 | Abtb2 | ankyrin repeat and BTB (POZ) domain containing | −0.47 | 4.37E−02 |
| | | 2 | | |
| 22271 | Upp1 | uridine phosphorylase 1 | −0.52 | 4.38E−02 |
| 381236 | Lipo3 | lipase, member O3 | −0.42 | 4.42E−02 |
| 66676 | Tmed7 | transmembrane p24 trafficking protein 7 | −0.24 | 4.43E−02 |
| 67041 | Oxct1 | 3-oxoacid CoA transferase 1 | −0.25 | 4.45E−02 |
| 12663 | Chml | choroideremia-like | −0.27 | 4.46E−02 |
| 21929 | Tnfaip3 | tumor necrosis factor, alpha-induced protein 3 | −0.38 | 4.46E−02 |
| 69036 | Zg16 | zymogen granule protein 16 | −0.30 | 4.47E−02 |
| 12313 | Calm1 | calmodulin 1 | −0.21 | 4.54E−02 |
| 71927 | Itfg1 | integrin alpha FG-GAP repeat containing 1 | −0.29 | 4.55E−02 |
| 140780 | Bmp2k | BMP2 inducible kinase | −0.28 | 4.57E−02 |
| 218442 | Serinc5 | serine incorporator 5 | −0.27 | 4.57E−02 |
| 98267 | Stk17b | serine/threonine kinase 17b (apoptosis-inducing) | −0.43 | 4.63E−02 |
| 225326 | Pik3c3 | phosphatidylinositol 3-kinase catalytic subunit | −0.32 | 4.66E−02 |
| | | type 3 | | |
| 68537 | Mrpl13 | mitochondrial ribosomal protein L13 | −0.32 | 4.75E−02 |
| 319148 | H3c3 | H3 clustered histone 3 | −0.52 | 4.81E−02 |
| 22612 | Yes1 | YES proto-oncogene 1, Src family tyrosine kinase | −0.26 | 4.81E−02 |
| 665769 | Gsdmcl2 | gasdermin C-like 2 | −0.45 | 4.90E−02 |
| 407795 | Smim31 | small integral membrane protein 31 | −0.35 | 4.90E−02 |

TABLE S4A
Levels of N-methylserotonin (ng) released by bacterial monocultures in TYG medium,
Wilkins-Chalgren anaerobe broth or MEGA medium 2.0 containing orange fiber.

| | | | | | | Wilkins- | MEGA |
|---|---|---|---|---|---|---|---|
| | | TYG medium | | | | Chalgren | Medium 2.0 |
| Bacterial strain | Accession Number | 8 hrs | 24 hrs | 48 hrs | 72 hrs | 72 hrs | 72 hrs |
| Bacteroides caccae | PRJEB3730 | 0.2 ± 0.1 | 0.3 ± 0.1 | 0.5 ± 0.2 | 1.0 ± 0.1 | 0.9 ± 0.2 | 1.2 ± 0.1 |
| Bacteroides | PRJEB3735 | 0.3 ± 0.1 | 0.3 ± 0.1 | 0.6 ± 0.1 | 0.9 ± 0.2 | 1 ± 0.2 | 1.0 ± 0.2 |
| cellulosilyticus | | | | | | | |
| Bacteroides | PRJEB3745 | 0.2 ± 0.1 | 0.2 ± 0.1 | 0.5 ± 0.1 | 1.0 ± 0.1 | 0.8 ± 0.1 | 0.9 ± 0.1 |
| thetaiotaomicron | | | | | | | |
| Bacteroides vulgatus | PRJEB3748 | 0.2 ± 0.2 | 0.3 ± 0.2 | 0.7 ± 0.1 | 1.1 ± 0.2 | 1.1 ± 0.3 | 1.2 ± 0.3 |
| Bacteroides ovatus | PRJEB3740 | 0.4 ± 0.1 | 1.4 ± 0.1 | 6.1 ± 0.5 | 28.7 ± 1.2 | 25.1 ± 1.4 | 1.2 ± 0.2 |
| (TSDC 17.2) | | | | | | | |
| Bacteroides | PRJEB3738 | 0.2 ± 0.1 | 0.2 ± 0.1 | 0.6 ± 0.1 | 1.0 ± 0.1 | 0.8 ± 0.1 | 1.3 ± 0.1 |
| massiliensis | | | | | | | |
| Bacteroides finegoldii | PRJEB3733 | 0.2 ± 0.1 | 0.3 ± 0.1 | 0.5 ± 0.1 | 1.0 ± 0.2 | 0.6 ± 0.2 | 12.9 ± 1.3 |
| Collinsella aerofaciens | PRJEB3760 | 0.3 ± 0.2 | 0.3 ± 0.2 | 0.6 ± 0.2 | 1.3 ± 0.1 | 0.8 ± 0.1 | 5.7 ± 1.7 |
| Escherichia coli | PRJEB3766 | 0.2 ± 0.1 | 0.4 ± 0.1 | 0.6 ± 0.1 | 0.9 ± 0.2 | 0.7 ± 0.1 | 1.3 ± 0.4 |
| Odoribacter | PRJEB3768 | 0.2 ± 0.2 | 0.2 ± 0.2 | 0.4 ± 0.1 | 1.0 ± 0.1 | 0.9 ± 0.1 | 1.1 ± 0.2 |
| splanchnicus | | | | | | | |
| Parabacteroides | PRJEB3769 | 0.3 ± 0.1 | 1.0 ± 0.1 | 7.5 ± 0.7 | 24.3 ± 2.4 | 22.1 ± 0.7 | 24.8 ± 2.1 |
| distasonis | | | | | | | |
| Ruminococcaceae sp. | PRJEB3772 | 0.2 ± 0.1 | 0.2 ± 0.1 | 0.4 ± 0.1 | 0.6 ± 0.1 | 0.6 ± 0.1 | 1.1 ± 0.1 |
| Ruminococcus albus | PRJEB3773 | 0.2 ± 0.1 | 0.2 ± 0.1 | 0.3 ± 0.1 | 0.6 ± 0.1 | 0.5 ± 0.1 | 0.9 ± 0.3 |
| Subdoligranulum | PRJEB3780 | 0.2 ± 0.1 | 0.2 ± 0.1 | 0.5 ± 0.1 | 0.9 ± 0.1 | 0.8 ± 0.1 | 1.2 ± 0.1 |
| variabile | | | | | | | |
| orange fiber alone | — | 0.2 ± 0.1 | 0.2 ± 0.1 | 0.3 ± 0.1 | 0.3 ± 0.1 | 0.5 ± 0.1 | 0.4 ± 0.1 |
| no orange fiber | — | Not Detected | Not Detected | Not Detected | Not Detected | Not Detected | Not Detected |
| | | (ND) | (ND) | (ND) | (ND) | (ND) | (ND) |

TABLE S4B
Screening of additional bacterial strains for
N-methylserotonin release from orange fiber.

| | N-methylserotonin |
|---|---|
| Bacterial strain | levels at 72 h (ng) |
| Anaerococcus vaginalis TSDC20 | 0.6 ± 0.1 |
| Anaerofustis stercorihominis TSDC20 | 0.7 ± 0.1 |
| Bacteroides caccae TSDC20 | 0.7 ± 0.1 |
| Bacteroides cellulosilyticus TSDC20 | 0.6 ± 0.2 |
| Bacteroides fragilis TSDC20 | 0.7 ± 0.1 |
| Bacteroides thetaiotaomicron TSDC20 | 0.7 ± 0.1 |
| Bacteroides uniformis TSDC20-1 | 0.6 ± 0.2 |
| Bacteroides uniformis TSDC20-2 | 0.8 ± 0.2 |
| Bifidobacterium longum TSDC20 | 0.9 ± 0.1 |
| Bifidobacterium TSDC20 | 0.7 ± 0.1 |
| Clostridiales TSDC20 | 0.6 ± 0.2 |
| Clostridium bolteae TSDC20 | 0.6 ± 0.2 |
| Clostridium hylemonae TSDC20 | 0.6 ± 0.1 |
| Clostridium scindens TSDC20 | 0.8 ± 0.2 |
| Dialister invisus TSDC20 | 0.7 ± 0.1 |
| Dorea longicatena TSDC20 | 0.6 ± 0.1 |
| Eggerthella lenta TSDC20 | 0.6 ± 0.1 |
| Escherichia coli TSDC20 | 0.7 ± 0.1 |
| Finegoldia magna TSDC20 | 0.6 ± 0.1 |
| Ruminococcus gnavus TSDC20 | 0.6 ± 0.1 |
| Subdoligranulum variabile TSDC20 | 0.8 ± 0.2 |
| Veillonella parvula TSDC20 | 0.7 ± 0.2 |
| Veillonella TSDC20-1 | 0.6 ± 0.2 |
| Veillonella TSDC20-2 | 0.6 ± 0.1 |
| orange fiber alone | 0.3 ± 0.1 |

TABLE S4C
Additional control experiments of bacterial N-methylserotonin release.

| | | N-methylserotonin levels (ng) | | | |
|---|---|---|---|---|---|
| | | Heat | Conditioned | Cellular | |
| Bacterial strain | Medium | inactivated | media | extract | Control |
| Bacteroides ovatus | Mega 2.0 | ND | ND | ND | |
| (TSDC 17.2) | | | | | |
| Bacteroides | Mega 2.0 | ND | ND | ND | |
| finegoldii | | | | | |
| Collinsella | Mega 2.0 | ND | ND | ND | |
| aerofaciens | | | | | |
| Parabacteroides | Mega 2.0 | ND | ND | ND | |
| distasonis | | | | | |
| Bacteroides ovatus | TYG | ND | ND | ND | |
| (TSDC 17.2) | | | | | |
| Bacteroides | TYG | ND | ND | ND | |
| finegoldii | | | | | |
| Collinsella | TYG | ND | ND | ND | |
| aerofaciens | | | | | |
| Parabacteroides | TYG | ND | ND | ND | |
| distasonis | | | | | |
| Bacteroides ovatus | Mega 2.0 | 0.3 ± 0.1 | 0.3 ± 0.1 | 0.4 ± 0.1 | |
| (TSDC 17.2) | (+orange fiber) | | | | |
| Bacteroides | Mega 2.0 | 0.3 ± 0.1 | 0.3 ± 0.1 | 0.3 ± 0.1 | |
| finegoldii | (+orange fiber) | | | | |
| Collinsella | Mega 2.0 | 0.3 ± 0.1 | 0.3 ± 0.1 | 0.3 ± 0.1 | |
| aerofaciens | (+orange fiber) | | | | |
| Parabacteroides | Mega 2.0 | 0.4 ± 0.1 | 0.3 ± 0.1 | 0.3 ± 0.1 | |
| distasonis | (+orange fiber) | | | | |
| Bacteroides ovatus | TYG (+orange | 0.3 ± 0.1 | 0.3 ± 0.1 | 0.3 ± 0.1 | |
| (TSDC 17.2) | fiber) | | | | |
| Bacteroides | TYG (+orange | 0.3 ± 0.1 | 0.3 ± 0.1 | 0.4 ± 0.1 | |
| finegoldii | fiber) | | | | |
| Collinsella | TYG (+orange | 0.2 ± 0.1 | 0.3 ± 0.1 | 0.3 ± 0.1 | |
| aerofaciens | fiber) | | | | |
| Parabacteroides | TYG (+orange | 0.3 ± 0.1 | 0.3 ± 0.1 | 0.2 ± 0.1 | |
| distasonis | fiber) | | | | |
| Orange | Mega 2.0 | | | | 0.4 ± 0.1 |
| fiber alone | (+orange fiber) | | | | |
| Orange | TYG (+orange | | | | 0.3 ± 0.1 |
| fiber alone | fiber) | | | | |

TABLE S4D
N-methylserotonin degradation test.

| | Starting N- | Ending N-methylserotonin level |
|---|---|---|
| Bacterial strain | methylserotonin level | after 72 h incubation (ng) |
| Bacteroides caccae | 50 ng | 49 ± 1 |
| Bacteroides cellulosilyticus | 50 ng | 50 ± 2 |
| Bacteroides thetaiotaomicron | 50 ng | 49 ± 2 |
| Bacteroides vulgatus | 50 ng | 50 ± 1 |
| Bacteroides ovatus (TSDC 17.2) | 50 ng | 49 ± 1 |
| Bacteroides massiliensis | 50 ng | 49 ± 2 |
| Bacteroides finegoldii | 50 ng | 50 ± 1 |
| Collinsella aerofaciens | 50 ng | 50 ± 1 |
| Escherichia coli | 50 ng | 49 ± 1 |
| Odoribacter splanchnicus | 50 ng | 50 ± 2 |
| Parabacteroides distasonis | 50 ng | 49 ± 2 |
| Ruminococcaceae sp. | 50 ng | 50 ± 1 |
| Ruminococcus albus | 50 ng | 49 ± 1 |
| Subdoligranulum variabile | 50 ng | 50 ± 1 |

TABLE S4E
Release of N-methylserotonin via enzymatic
reaction after 72 hours.

| Enzyme | N-methylserotonin levels at 72 h (ng) |
|---|---|
| Cellulase | 2727.9 ± 25.5 |
| Xylanase | 1045.5 ± 5.5 |
| Hemicellulose | 860.9 ± 12.8 |
| beta-Xylanase | 641.7 ± 21.9 |
| Alginate Lyase | 563.8 ± 5.9 |
| endo-Inulinase | 522.3 ± 19.4 |
| exo-Inulinase | 514.0 ± 9.0 |
| beta-Mannanase | 487.4 ± 6.5 |
| endo 1,5 alpha L-arabinanase | 454.4 ± 10.3 |
| Lichenase | 433.7 ± 1.9 |
| endo 1,4 beta D-galactanase | 395.8 ± 5.9 |
| endo-Polygalacturonananase (M2) | 393.0 ± 11.2 |
| Amylase | 61.5 ± 1.9 |
| Control (blank) | 0 ± 0 |
| Control (Fiber alone) | 15.2 ± 3.2 |

TABLE S4F
Recovery of N-methylserotonin from orange fiber after repeated rounds
of methanol extraction, compared to recovery of a closely related
spike-in compound (2-methylserotonin) under the same conditions.

| Round of Extraction | N-methylserotonin recovered | 2-methylserotonin remaining |
|---|---|---|
| Starting materials | 50 mg orange fiber | 200 ng |
| No extraction | | 200 ng |
| Extraction 1 | 16 ng | 190 ng |
| Extraction 2 | 15 ng | 10 ng |
| Extraction 3 | 16 ng | 0 ng |
| Extraction 4 | 15 ng | 0 ng |
| Extraction 5 | 14 ng | 0 ng |
| Extraction 6 | 13 ng | 0 ng |
| Extraction 7 | 12 ng | 0 ng |
| Extraction 8 | 10 ng | 0 ng |
| Extraction 9 | 11 ng | 0 ng |
| Extraction 10 | 10 ng | 0 ng |
| Extraction 11 | 9 ng | 0 ng |
| Extraction 12 | 9 ng | 0 ng |
| Extraction 13 | 8 ng | 0 ng |
| Extraction 14 | 7 ng | 0 ng |
| Extraction 15 | 6 ng | 0 ng |

TABLE S4G
Screening of additional Bacteroides ovatus strains for N-methylserotonin
release from orange fiber using TYG media with hemin.

| Bacterial strain | N-methylserotonin levels at 72 h (ng) |
|---|---|
| Bacteroides ovatus (TSDC 17.2) | 33.3 ± 3 |
| Bacteroides ovatus VPI-C2-26 | 10.6 ± 0.1 |
| Bacteroides ovatus WH713 | 8.2 ± 0.2 |
| Bacteroides ovatus WH514 | 5.5 ± 0.1 |
| Bacteroides ovatus WH604 | 5.0 ± 0.3 |
| Bacteroides ovatus VPI-C1-45 | 4.9 ± 0.2 |
| Bacteroides ovatus VPI-B4-11 | 4.9 ± 0.1 |
| Bacteroides ovatus WH208 | 4.7 ± 0.1 |
| Bacteroides ovatus VPI-C16-22 | 4.5 ± 0.2 |
| Bacteroides ovatus WH711 | 4.3 ± 0.1 |
| Bacteroides ovatus VPI-435 | 3.9 ± 0.2 |
| Bacteroides ovatus 115 | 2.8 ± 0.1 |

TABLE S4H
Screening of additional Bacteroides ovatus strains for N-methylserotonin
release from orange fiber using TYG media without hemin.

| Bacterial strain | N-methylserotonin levels at 72 h (ng) |
|---|---|
| Bacteroides ovatus (TSDC 17.2) | 0.9 ± 0.08 |
| Bacteroides ovatus VPI-C2-26 | 0.7 ± 0.03 |
| Bacteroides ovatus WH713 | 0.5 ± 0.09 |
| Bacteroides ovatus WH514 | 0.7 ± 0.08 |
| Bacteroides ovatus WH604 | 0.6 ± 0.03 |
| Bacteroides ovatus VPI-C1-45 | 1.1 ± 0.07 |
| Bacteroides ovatus VPI-B4-11 | 0.9 ± 0.09 |
| Bacteroides ovatus WH208 | 0.7 ± 0.05 |
| Bacteroides ovatus VPI-C16-22 | 0.8 ± 0.01 |
| Bacteroides ovatus WH711 | 0.7 ± 0.05 |
| Bacteroides ovatus VPI-435 | 0.7 ± 0.03 |
| Bacteroides ovatus 115 | 0.7 ± 0.03 |

TABLE S5
Absolute abundances of fecal bacterial community members
measured at experimental day 21 (mean ± SD).

| | Absolute abundance [genome equivalents (10\hat{ }6)/mg feces] | | |
|---|---|---|---|
| | 14-member | 10-member | 4-member |
| Bacteria | consortium | consortium | consortium |
| Bacteroides caccae | 8.7 ± 4 | 10.48 ± 0.61 | — |
| Bacteroides finegoldii | 0.2 ± 0.1 | — | 2.0 ± 0.7 |
| Bacteroides cellulosilyticus | 30.1 ± 13.3 | 33.1 ± 5 | — |
| Bacteroides massiliensis | 0.7 ± 0.3 | 0.9 ± 0.3 | — |
| Bacteroides ovatus (TSDC 17.2) | 20.3 ± 8 | — | 56.9 ± 11.3 |
| Bacteroides thetaiotaomicron | 29 ± 9.1 | 33.9 ± 7 | — |
| Bacteroides vulgatus | 18.3 ± 14.7 | 28.5 ± 5.5 | — |
| Collinsella aerofaciens | 0.4 ± 0.1 | — | 0.9 ± 0.1 |
| Escherichia coli | 10.2 ± 3 | 7.4 ± 0.5 | — |
| Odoribacter splanchnicus | 1.2 ± 0.3 | 1.4 ± 0.2 | — |
| Parabacteroides distasonis | 6.1 ± 2.4 | — | 6.6 ± 1.7 |
| Ruminococcaceae | 2.2 ± 0.3 | 3.7 ± 0.5 | — |
| Ruminococcus albus | 0.02 ± 0.01 | 0.02 ± 0.01 | — |
| Subdoligranulum variabile | 5 ± 1.3 | 5.3 ± 0.6 | — |

TABLE S6A
B. ovatus TSDC 17.2 genes, arranged by log2fold-change under the permissive release
condition (TYG medium containing hemin, with versus without orange fiber).

| | | | Release | | No N-methylserotonin release | | | |
|---|---|---|---|---|---|---|---|---|
| | | | B. ovatus strain | | | | | |
| | | | 17.2 | | 17.2 | | 17.2 | |
| | | | Medium condition | | | | | |
| | | | TYG | | TYG (no hemin) | | MEGA | |
| Gene | PUL | Annotation | log2fold | padj | log2fold | padj | log2fold | padj |
| Bova172_1948 | Predicted | Unsaturated glucuronyl hydrolase | 6.08 | 1.1E−53 | 0.23 | 4.9E−01 | 0.63 | 1.9E−01 |
| | PUL 27 | | | | | | | |
| Bova172_1644 | — | Putative DNA methylase | 5.52 | 2.3E−20 | −3.39 | 2.2E−13 | 0.44 | 3.4E−01 |
| Bova172_1942 | Predicted | SusD-like family | 4.79 | 7.9E−27 | 0.45 | 2.9E−01 | 0.58 | 2.5E−01 |
| | PUL 27 | | | | | | | |
| Bova172_5258 | — | Adenine-specific methyltransferase (EC | 4.26 | 5.1E−62 | −2.16 | 2.7E−22 | −0.85 | 1.6E−03 |
| | | 2.1.1.72) | | | | | | |
| Bova172_1939 | Predicted | SusC-like family | 4.26 | 4.5E−23 | 0.10 | 7.6E−01 | 0.67 | 1.6E−01 |
| | PUL 27 | | | | | | | |
| Bova172_555 | — | Putative glycosyl hydrolase of unknown | 4.02 | 1.9E−11 | −0.12 | 7.1E−01 | 0.58 | 2.6E−01 |
| | | function; Aceric acid hydrolase | | | | | | |
| Bova172_5248 | — | DNA-directed RNA polymerase beta | 3.66 | 6.7E−125 | −2.36 | 3.7E−48 | −0.44 | 1.8E−02 |
| | | subunit (EC 2.7.7.6) | | | | | | |
| Bova172_4180 | — | Ribonucleotide reductase of class II | 3.50 | 0.0E+00 | 0.11 | 1.3E−01 | 0.08 | 6.0E−01 |
| | | (coenzyme B12-dependent) (EC | | | | | | |
| | | 1.17.4.1) | | | | | | |
| Bova172_1037 | — | α-glucosidase | 3.17 | 3.6E−13 | 0.44 | 1.8E−01 | 0.35 | 4.6E−01 |
| Bova172_5251 | — | Helicase-related protein | 3.08 | 1.6E−67 | −1.96 | 5.4E−21 | 0.05 | 8.7E−01 |
| Bova172_5250 | — | putative recombination protein | 3.07 | 6.2E−28 | −2.00 | 1.2E−10 | −0.51 | 1.4E−01 |
| Bova172_1575 | — | Conjugative transposon protein TraN | 2.97 | 2.8E−05 | 4.63 | 8.9E−30 | 0.03 | 9.6E−01 |
| Bova172_5275 | — | Integrase | 2.95 | 2.0E−30 | −1.92 | 6.3E−14 | 0.09 | 8.4E−01 |
| Bova172_4496 | — | Integrase | 2.88 | 1.0E−05 | −0.98 | 2.0E−02 | 0.67 | 2.2E−01 |
| Bova172_546 | Predicted | SusC-like family | 2.71 | 5.8E−17 | −0.65 | 1.7E−05 | 0.38 | 3.4E−01 |
| | PUL 2 | | | | | | | |
| Bova172_5263 | — | SAM-dependent methyltransferase | 2.70 | 1.3E−19 | −1.30 | 5.1E−07 | −0.14 | 7.3E−01 |
| Bova172_374 | CAZyme | Sialic acid-specific 9-O-acetylesterase | 2.69 | 3.9E−07 | 0.34 | 3.9E−01 | 0.37 | 4.6E−01 |
| | cluster 1 | | | | | | | |
| Bova172_1992 | Predicted | β-D-glycosidase | 2.68 | 1.7E−16 | 0.40 | 1.9E−01 | −0.15 | 7.7E−01 |
| | PUL 29 | | | | | | | |
| Bova172_770 | — | Phage endolysin | 2.66 | 2.2E−04 | −0.72 | 1.7E−01 | 0.48 | 1.0E+00 |
| Bova172_4622 | — | Transposase | 2.66 | 6.6E−09 | 0.70 | 1.0E−01 | 0.59 | 2.1E−01 |
| Bova172_1993 | Predicted | α-L/β-D-glycosidase | 2.55 | 6.2E−17 | −0.71 | 3.7E−03 | −0.01 | 9.8E−01 |
| | PUL 29 | | | | | | | |
| Bova172_1576 | — | Conjugative transposon protein TraM | 2.54 | 9.7E−07 | −5.03 | 4.6E−51 | −0.28 | 5.8E−01 |
| Bova172_1972 | Predicted | Unsaturated rhamnogalacturonyl | 2.43 | 1.5E−05 | 0.22 | 6.6E−01 | −0.25 | 6.3E−01 |
| | PUL 28 | hydrolase | | | | | | |
| Bova172_1668 | — | Transposase | 2.41 | 1.7E−23 | −1.92 | 2.2E−15 | 0.32 | 2.7E−01 |
| Bova172_589 | Predicted | RG-II backbone hydrolase | 2.39 | 3.4E−08 | −0.36 | 3.6E−01 | 0.36 | 4.7E−01 |
| | PUL 4 | | | | | | | |
| Bova172_1036 | — | Corrinoid methyltransferase protein | 2.37 | 2.5E−12 | −0.48 | 9.2E−02 | 0.52 | 1.9E−01 |
| Bova172_3019 | — | Two-component transcriptional response | 2.35 | 1.6E−69 | −1.17 | 2.1E−29 | 0.14 | 4.6E−01 |
| | | regulator, LuxR family | | | | | | |
| Bova172_3798 | — | Transcriptional regulator, MarR family | 2.32 | 2.0E−47 | −0.15 | 1.9E−01 | 0.30 | 2.9E−01 |
| Bova172_1953 | Predicted | α-galacturonidase | 2.31 | 1.5E−06 | −0.35 | 4.7E−01 | 0.54 | 2.8E−01 |
| | PUL 27 | | | | | | | |
| Bova172_5265 | — | PUA-PAPS reductase like fusion | 2.30 | 1.1E−16 | −0.72 | 3.1E−02 | −0.81 | 3.4E−02 |
| Bova172_4528 | — | Integrase | 2.30 | 3.0E−06 | −0.58 | 7.5E−02 | −0.05 | 9.4E−01 |
| Bova172_5193 | — | Phage endolysin | 2.28 | 3.5E−06 | −0.87 | 4.6E−02 | 0.21 | 6.8E−01 |
| Bova172_1971 | Predicted | L-rhamnose mutarotase (EC 5.1.3.32) | 2.26 | 1.3E−03 | −1.86 | 2.2E−03 | 0.44 | 1.0E+00 |
| | PUL 28 | | | | | | | |
| Bova172_5293 | — | Phage terminase, large subunit | 2.25 | 2.7E−06 | −0.19 | 6.4E−01 | −0.77 | 1.1E−01 |
| Bova172_5447 | — | Iron-regulated protein A precursor | 2.23 | 1.6E−04 | −1.20 | 4.1E−03 | −0.34 | 4.6E−01 |
| Bova172_1664 | Predicted | OmpR-type DNA-binding response | 2.21 | 1.3E−19 | −0.32 | 4.4E−02 | −0.31 | 2.2E−01 |
| | PUL 23 | regulator | | | | | | |
| Bova172_3000 | Predicted | Putative anti-sigma factor | 2.19 | 1.8E−107 | −6.51 | 0.0E+00 | −0.08 | 6.6E−01 |
| | PUL 59 | | | | | | | |
| Bova172_590 | Predicted | Endo-apiosidase | 2.15 | 1.1E−07 | −0.11 | 7.7E−01 | 0.65 | 2.1E−01 |
| | PUL 4 | | | | | | | |
| Bova172_5421 | — | Protein DUF2149, predicted transporter | 2.14 | 2.8E−06 | −0.05 | 8.9E−01 | −0.56 | 2.0E−01 |
| | | component | | | | | | |
| Bova172_4939 | Predicted | Putative anti-sigma factor | 2.11 | 5.4E−10 | −1.12 | 2.1E−06 | 0.16 | 7.0E−01 |
| | PUL 105 | | | | | | | |
| Bova172_420 | — | Single-stranded DNA-binding protein | 2.10 | 1.3E−06 | −2.20 | 4.7E−09 | 0.39 | 4.0E−01 |
| Bova172_643 | — | Co-activator of prophage gene | 2.07 | 7.2E−35 | −0.35 | 3.6E−03 | −0.77 | 2.4E−02 |
| | | expression IbrB | | | | | | |
| Bova172_1996 | Predicted | RGI specific α-galacturonidase | 2.06 | 1.3E−09 | −0.37 | 2.0E−01 | −0.26 | 5.6E−01 |
| | PUL 29 | | | | | | | |
| Bova172_3984 | — | Chromosome (plasmid) partitioning | 2.04 | 8.1E−05 | 0.29 | 3.7E−01 | −0.13 | 8.0E−01 |
| | | protein ParB | | | | | | |
| Bova172_545 | Predicted | SusD-like family | 2.00 | 2.7E−05 | −0.20 | 4.3E−01 | −0.07 | 8.8E−01 |
| | PUL 2 | | | | | | | |
| Bova172_1224 | Predicted | α-galactosidase | 1.96 | 1.3E−10 | 0.13 | 6.1E−01 | −0.09 | 8.7E−01 |
| | PUL 13 | | | | | | | |
| Bova172_5291 | — | putative DNA methylase | 1.96 | 3.8E−06 | −0.11 | 8.1E−01 | −0.45 | 3.5E−01 |
| Bova172_2884 | Predicted | SusC-like family | 1.97 | 3.2E−114 | −3.81 | 0.0E+00 | −0.55 | 1.7E−04 |
| | PUL 53 | | | | | | | |
| Bova172_1407 | — | RNA polymerase ECF-type sigma factor | 1.96 | 2.2E−139 | −3.86 | 0.0E+00 | −1.87 | 4.8E−96 |
| Bova172_232 | — | D-xylose proton-symporter XylE | 1.95 | 1.2E−17 | −0.10 | 6.7E−01 | 0.36 | 2.4E−01 |
| Bova172_5094 | — | Maturase-related protein | 1.95 | 4.9E−03 | −0.52 | 3.5E−01 | 0.50 | 1.0E+00 |
| Bova172_66 | — | ATP synthase beta chain (EC 3.6.3.14) | 1.94 | 1.4E−131 | 0.07 | 4.3E−01 | 0.02 | 9.3E−01 |
| Bova172_1976 | Predicted | Polysaccharide lyase | 1.94 | 1.5E−05 | −0.58 | 1.2E−01 | 0.45 | 3.6E−01 |
| | PUL 28 | | | | | | | |
| Bova172_5092 | — | Maturase-related protein | 1.93 | 5.1E−03 | −0.91 | 1.4E−01 | 0.84 | 1.0E+00 |
| Bova172_665 | — | Possible exported heavy-metal binding | 1.91 | 1.9E−20 | −0.48 | 3.9E−03 | −0.82 | 1.6E−02 |
| | | protein | | | | | | |
| Bova172_2537 | Predicted | outer membrane protein SusE | 1.84 | 8.8E−15 | 0.01 | 9.8E−01 | −1.12 | 3.4E−02 |
| | PUL 45 | | | | | | | |
| Bova172_4940 | Predicted | SusC-like family | 1.83 | 1.7E−06 | 0.41 | 1.2E−01 | 0.24 | 5.9E−01 |
| | PUL 105 | | | | | | | |
| Bova172_3245 | Predicted | beta-glucosidase (EC 3.2.1.21) | 1.81 | 7.1E−12 | −1.91 | 1.3E−28 | 0.50 | 1.7E−01 |
| | PUL 70 | | | | | | | |
| Bova172_1227 | Predicted | SusD-like family | 1.80 | 2.4E−15 | −0.26 | 2.2E−01 | 0.35 | 3.3E−01 |
| | PUL 13 | | | | | | | |
| Bova172_3817 | — | Transposase | 1.78 | 5.4E−07 | −0.07 | 8.1E−01 | 0.50 | 2.7E−01 |
| Bova172_1230 | Predicted | RNA polymerase ECF-type sigma factor | 1.77 | 2.0E−04 | −1.11 | 1.8E−02 | −0.07 | 8.9E−01 |
| | PUL 13 | | | | | | | |
| Bova172_4274 | — | Transposase | 1.76 | 9.3E−03 | 0.05 | 9.1E−01 | 0.03 | 1.0E+00 |
| Bova172_1645 | — | Putative DNA methylase | 1.76 | 9.7E−03 | −0.91 | 1.4E−01 | 0.87 | 1.0E+00 |
| Bova172_5442 | — | Putative cytochrome C-type biogenesis | 1.73 | 1.8E−03 | −0.24 | 5.8E−01 | 0.78 | 1.3E−01 |
| | | protein | | | | | | |
| Bova172_5579 | — | 4-diphosphocytidyl-2-C-methyl-D- | 1.67 | 9.0E−220 | −4.18 | 0.0E+00 | −0.03 | 7.2E−01 |
| | | erythritol kinase (EC 2.7.1.148) | | | | | | |
| Bova172_4162 | — | Transcriptional regulator, MarR family | 1.66 | 9.5E−15 | −0.08 | 6.0E−01 | −1.14 | 3.3E−03 |
| Bova172_1090 | CAZyme | Rhamnogalacturonides degradation | 1.66 | 1.1E−07 | −1.98 | 4.7E−14 | 0.41 | 3.4E−01 |
| | cluster 2 | protein RhiN | | | | | | |
| Bova172_4145 | — | Transcriptional regulator | 1.64 | 8.0E−04 | 0.36 | 2.8E−01 | 0.41 | 3.8E−01 |
| Bova172_561 | Predicted | Cytochrome c-type biogenesis protein | 1.64 | 8.2E−07 | −2.19 | 9.2E−21 | 0.24 | 5.8E−01 |
| | PUL 3 | DsbD, protein-disulfide reductase | | | | | | |
| Bova172_5605 | — | Integral membrane protein | 1.63 | 2.2E−03 | −1.32 | 2.8E−03 | 0.64 | 1.9E−01 |
| Bova172_3118 | — | Transcriptional regulator, AraC family | 1.62 | 4.4E−09 | −1.34 | 3.6E−10 | 0.41 | 1.0E−01 |
| Bova172_3829 | — | Aquaporin Z | 1.61 | 2.5E−04 | −0.46 | 1.9E−01 | −0.07 | 8.9E−01 |
| Bova172_2536 | Predicted | SusD-like family | 1.61 | 6.8E−18 | 0.10 | 4.9E−01 | −1.32 | 1.3E−02 |
| | PUL 45 | | | | | | | |
| Bova172_4293 | — | Aconitate hydratase (EC 4.2.1.3) | 1.59 | 1.2E−107 | −0.14 | 1.2E−02 | −0.84 | 1.0E−09 |
| Bova172_1954 | Predicted | Rhamnogalacturonan lyase | 1.59 | 3.0E−21 | −0.75 | 2.5E−04 | 0.16 | 5.9E−01 |
| | PUL 28 | | | | | | | |
| Bova172_2613 | Predicted | Endo-1,4-beta-xylanase (EC 3.2.1.8) | 1.58 | 1.6E−05 | −0.15 | 5.3E−01 | −0.30 | 5.4E−01 |
| | PUL 47 | | | | | | | |
| Bova172_1253 | — | Mobilization protein BmpH | 1.57 | 4.3E−03 | −1.86 | 3.5E−05 | 0.48 | 3.4E−01 |
| Bova172_3738 | — | Integrase IntN1 | 1.57 | 2.6E−47 | −1.75 | 2.3E−69 | 0.15 | 3.3E−01 |
| Bova172_1300 | — | Putative helicase | 1.56 | 3.2E−24 | −0.46 | 1.2E−04 | 0.24 | 2.8E−01 |
| Bova172_2535 | Predicted | SusC-like family | 1.55 | 5.3E−25 | −0.15 | 2.4E−01 | −1.20 | 6.1E−05 |
| | PUL 45 | | | | | | | |
| Bova172_1228 | Predicted | SusC-like family | 1.53 | 1.6E−29 | −0.38 | 7.4E−03 | 0.14 | 6.0E−01 |
| | PUL 13 | | | | | | | |
| Bova172_3860 | Predicted | Putative anti-sigma factor | 1.52 | 1.2E−59 | −2.92 | 1.4E−209 | −0.22 | 7.0E−02 |
| | PUL 84 | | | | | | | |
| Bova172_387 | — | DNA primase, TraP-type | 1.49 | 1.1E−04 | 0.05 | 8.8E−01 | 0.50 | 2.7E−01 |
| Bova172_5394 | Predicted | Putative anti-sigma factor | 1.49 | 1.5E−26 | −2.91 | 4.7E−91 | 0.03 | 9.2E−01 |
| | PUL 108 | | | | | | | |
| Bova172_845 | — | Tyrosine type site-specific recombinase | 1.47 | 2.6E−02 | 0.25 | 6.2E−01 | 0.14 | 1.0E+00 |
| Bova172_2802 | — | Colicin V production protein | 1.48 | 5.7E−10 | 0.05 | 7.3E−01 | −0.31 | 4.8E−01 |
| Bova172_2696 | — | Oxidoreductase, aldo/keto reductase | 1.47 | 1.1E−02 | −0.68 | 1.0E−01 | 0.32 | 5.0E−01 |
| | | family | | | | | | |
| Bova172_3862 | Predicted | SusC-like family | 1.45 | 7.6E−18 | −1.85 | 8.0E−52 | −0.96 | 2.4E−06 |
| | PUL 84 | | | | | | | |
| Bova172_1110 | Predicted | SusC-like family | 1.42 | 2.2E−03 | 0.22 | 4.9E−01 | −0.03 | 9.6E−01 |
| | PUL 10 | | | | | | | |
| Bova172_765 | — | Tyrosine type site-specific recombinase | 1.42 | 8.6E−05 | −0.15 | 6.0E−01 | 0.00 | 1.0E+00 |
| Bova172_5202 | — | putative ryanodine receptor | 1.36 | 2.1E−14 | −2.65 | 5.0E−149 | 0.14 | 6.4E−01 |
| Bova172_358 | — | putative membrane protein | 1.36 | 5.6E−05 | −0.67 | 1.2E−02 | 0.14 | 7.6E−01 |
| Bova172_2955 | Predicted | putative anti-sigma factor | 1.35 | 3.0E−40 | −6.11 | 0.0E+00 | 0.01 | 9.7E−01 |
| | PUL 57 | | | | | | | |
| Bova172_4067 | — | Integrase | 1.30 | 8.4E−03 | −0.27 | 4.9E−01 | 0.32 | 5.3E−01 |
| Bova172_1981 | Predicted | SusC-like family | 1.29 | 5.6E−03 | 0.46 | 1.9E−01 | −0.55 | 2.8E−01 |
| | PUL 28 | | | | | | | |
| Bova172_1417 | — | Phosphate transport regulator (distant | 1.27 | 3.1E−07 | 0.17 | 3.3E−01 | −1.25 | 6.7E−03 |
| | | homolog of PhoU) | | | | | | |
| Bova172_5405 | — | Two-component system sensor histidine | 1.25 | 1.0E−26 | 0.02 | 8.5E−01 | −0.57 | 6.3E−02 |
| | | kinase | | | | | | |
| Bova172_1313 | Predicted | Iron siderophore sensor protein | 1.24 | 3.3E−15 | −1.60 | 8.0E−31 | −0.08 | 8.3E−01 |
| | PUL 16 | | | | | | | |
| Bova172_465 | — | putative ribose phosphate | 1.24 | 2.3E−04 | −1.00 | 2.2E−07 | 0.13 | 7.7E−01 |
| | | pyrophosphokinase | | | | | | |
| Bova172_2464 | Predicted | Polygalacturonase (EC 3.2.1.15) | 1.22 | 1.4E−02 | −0.18 | 7.0E−01 | −0.04 | 9.5E−01 |
| | PUL 42 | | | | | | | |
| Bova172_3192 | Predicted | arylsulfatase A precursor | 1.21 | 5.2E−03 | −0.61 | 4.5E−02 | 0.20 | 7.0E−01 |
| | PUL 67 | | | | | | | |
| Bova172_1517 | — | Mg(2+)-transport-ATPase-associated | 1.21 | 2.7E−35 | −3.70 | 1.7E−163 | −0.20 | 2.5E−01 |
| | | protein MgtC | | | | | | |
| Bova172_2081 | Predicted | alpha-mannosidase | 1.21 | 1.2E−06 | −3.09 | 8.4E−47 | 0.28 | 5.5E−01 |
| | PUL 31 | | | | | | | |
| Bova172_1952 | Predicted | Rhamnogalacturonan lyase | 1.21 | 2.2E−04 | −2.14 | 1.6E−10 | 0.46 | 3.1E−01 |
| | PUL 27 | | | | | | | |
| Bova172_4599 | Predicted | Endo-1,4-beta-xylanase (EC 3.2.1.8) | 1.21 | 1.2E−04 | −1.23 | 3.3E−15 | 0.43 | 3.0E−01 |
| | PUL 96 | | | | | | | |
| Bova172_3870 | — | Hexokinase (EC 2.7.1.1) | 1.19 | 1.3E−18 | 0.10 | 4.0E−01 | −0.71 | 2.7E−03 |
| Bova172_1271 | Predicted | COG5434 Endopygalactorunase | 1.19 | 1.0E−02 | −0.44 | 3.5E−01 | 0.03 | 9.5E−01 |
| | PUL 15 | | | | | | | |
| Bova172_1980 | Predicted | SusD-like family | 1.18 | 2.7E−02 | 0.32 | 3.9E−01 | −0.04 | 9.5E−01 |
| | PUL 28 | | | | | | | |
| Bova172_4546 | — | Free methionine-(R)-sulfoxide reductase, | 1.18 | 6.4E−07 | 0.18 | 4.3E−01 | 0.09 | 8.4E−01 |
| | | contains GAF domain | | | | | | |
| Bova172_4934 | — | putative ferric aerobactin receptor | 1.17 | 3.5E−03 | 0.28 | 4.6E−01 | 0.21 | 6.6E−01 |
| Bova172_4585 | Predicted | beta-glucosidase (EC 3.2.1.21) | 1.16 | 3.4E−03 | −0.50 | 6.0E−02 | 0.00 | 9.9E−01 |
| | PUL 96 | | | | | | | |
| Bova172_4658 | — | conserved hypothetical protein | 1.16 | 1.3E−03 | −0.95 | 1.2E−04 | 0.48 | 3.2E−01 |
| Bova172_1695 | — | DNA primase, TraP-type | 1.13 | 3.6E−02 | −0.73 | 4.1E−02 | 0.03 | 1.0E+00 |
| Bova172_5629 | — | Putative mobilization protein BF0133 | 1.13 | 7.0E−07 | 0.25 | 1.4E−01 | 0.16 | 6.2E−01 |
| Bova172_2886 | — | Endonuclease/exonuclease/phosphatase | 1.13 | 2.1E−04 | 0.16 | 3.9E−01 | −1.00 | 2.3E−02 |
| | | family protein | | | | | | |
| Bova172_2999 | Predicted | SusC-like family | 1.13 | 4.1E−24 | −3.80 | 8.3E−241 | −0.25 | 1.5E−01 |
| | PUL 59 | | | | | | | |
| Bova172_3116 | — | RND efflux system, inner membrane | 1.11 | 1.7E−02 | 0.06 | 8.6E−01 | 0.16 | 7.5E−01 |
| | | transporter | | | | | | |
| Bova172_2720 | CAZyme | Arylsulfatase (EC 3.1.6.1) | 1.11 | 3.9E−02 | −0.35 | 4.8E−01 | 0.62 | 2.1E−01 |
| | cluster 5 | | | | | | | |
| Bova172_2982 | — | beta-galactosidase (EC 3.2.1.23) | 1.11 | 3.1E−02 | 0.22 | 5.6E−01 | 0.03 | 9.5E−01 |
| Bova172_2498 | Predicted | Putative anti-sigma factor | 1.10 | 3.5E−36 | −3.48 | 0.0E+00 | −0.55 | 7.6E−04 |
| | PUL 44 | | | | | | | |
| Bova172_4101 | — | UPF0102 protein YraN | 1.09 | 6.8E−17 | −1.07 | 2.0E−16 | 0.01 | 9.7E−01 |
| Bova172_1113 | Predicted | SusD-like family | 1.08 | 9.7E−03 | −0.19 | 5.9E−01 | 0.85 | 1.1E−01 |
| | PUL 10 | | | | | | | |
| Bova172_4079 | Predicted | SusC-like family | 1.08 | 6.6E−28 | −1.47 | 3.4E−81 | 0.20 | 2.6E−01 |
| | PUL 88 | | | | | | | |
| Bova172_2885 | Predicted | SusD-like family | 1.07 | 5.2E−12 | −2.40 | 1.2E−120 | −1.40 | 1.9E−06 |
| | PUL 53 | | | | | | | |
| Bova172_3987 | — | Acetyltransferase, GNAT family | 1.07 | 7.0E−07 | −1.26 | 7.7E−13 | −0.44 | 1.0E−01 |
| Bova172_2534 | Predicted | Glucan 1,4-alpha-glucosidase (EC | 1.07 | 6.8E−12 | 0.09 | 5.4E−01 | −0.23 | 3.7E−01 |
| | PUL 45 | 3.2.1.3) | | | | | | |
| Bova172_4436 | — | Mobile element protein | 1.06 | 1.1E−08 | −0.53 | 3.3E−04 | 0.36 | 3.0E−01 |
| Bova172_4958 | — | Biotin carboxyl carrier protein | 1.05 | 1.2E−08 | −0.17 | 2.8E−01 | −1.12 | 2.4E−03 |
| Bova172_4839 | — | NADH dehydrogenase (EC 1.6.99.3) | 1.04 | 6.0E−45 | −0.28 | 3.8E−05 | −0.68 | 7.6E−08 |
| Bova172_2469 | Predicted | SusC-like family | 1.02 | 4.7E−04 | −1.13 | 3.5E−09 | 0.37 | 3.2E−01 |
| | PUL 42 | | | | | | | |
| Bova172_4787 | Predicted | Putative anti-sigma factor | 1.02 | 1.0E−45 | −4.23 | 0.0E+00 | −0.89 | 1.1E−14 |
| | PUL 101 | | | | | | | |
| Bova172_453 | — | Putative DNA methylase | 1.01 | 1.2E−04 | −1.96 | 2.7E−23 | 0.06 | 9.0E−01 |
| Bova172_3196 | Predicted | putative beta-xylosidase | 1.01 | 9.3E−03 | 0.33 | 2.3E−01 | −0.04 | 9.5E−01 |
| | PUL 67 | | | | | | | |

| | No N-methylserotonin release | | | | | | | | | | | | |
|---|---|---|---|---|---|---|---|---|---|---|---|---|---|
| | B. ovatus strain | | | | | | | | | | | | |
| | 115 | | | | | | | | | | | | |
| | Medium condition | | | | | | | | | | | | |
| | TYG | | | VPI- | | | | VPI- | VPI- | | VPI- | | VPI- |
| Gene | log2fold | padj | 115 | C2-26 | WH713 | WH514 | WH604 | C1-45 | B4-11 | WH208 | C16-22 | WH711 | 435 |
| Bova172_1948 | 0.02 | 1.0E+00 | x | | x | x | x | x | x | x | x | x | x |
| Bova172_1644 | 0.02 | 1.0E+00 | x | | | | | | | x | x | x | |
| Bova172_1942 | −0.01 | 1.0E+00 | x | | | | x | x | x | | x | x | x |
| Bova172_5258 | | | | | | | | | | | | x | |
| Bova172_1939 | −0.06 | 1.0E+00 | x | | | | x | x | x | | x | x | x |
| Bova172_555 | | | | | | x | | x | x | x | x | x | x |
| Bova172_5248 | | | | | | | | | | | | x | |
| Bova172_4180 | −0.28 | 1.6E−01 | x | x | x | x | x | x | x | x | x | x | x |
| Bova172_1037 | −0.02 | 1.0E+00 | x | x | x | x | | x | x | x | x | x | x |
| Bova172_5251 | | | | | | | | | | | | x | |
| Bova172_5250 | | | | | | | | | | | | x | |
| Bova172_1575 | 0.00 | 1.0E+00 | x | | | x | | | | x | x | x | |
| Bova172_5275 | | | | | | | x | | | | | | |
| Bova172_4496 | −0.06 | 1.0E+00 | x | | | x | | x | x | x | x | x | x |
| Bova172_546 | | | | x | | x | x | | x | | x | x | x |
| Bova172_5263 | | | | | | | | | | | | | |
| Bova172_374 | 0.03 | 1.0E+00 | x | x | x | x | | x | x | x | x | x | x |
| Bova172_1992 | −0.01 | 1.0E+00 | x | | x | x | x | x | x | x | x | x | x |
| Bova172_770 | | | | | | x | | | | x | x | x | x |
| Bova172_4622 | — | | | | | x | | x | x | x | x | x | x |
| Bova172_1993 | 0.05 | 1.0E+00 | x | | x | x | x | x | x | x | x | x | x |
| Bova172_1576 | | | | | | | | | | x | x | x | |
| Bova172_1972 | −0.01 | 1.0E+00 | x | | | | | x | | x | x | x | x |
| Bova172_1668 | | | | | | x | | | | x | x | x | |
| Bova172_589 | −0.03 | 9.0E−01 | x | x | x | x | | x | x | x | x | x | x |
| Bova172_1036 | 0.04 | 1.0E+00 | x | x | x | x | | x | x | x | x | x | x |
| Bova172_3019 | −0.07 | 1.0E+00 | x | x | x | x | x | x | x | x | x | x | x |
| Bova172_3798 | 0.05 | 8.4E−01 | x | x | x | x | x | x | x | x | x | x | x |
| Bova172_1953 | −0.05 | 1.0E+00 | x | | x | | | x | | x | x | x | x |
| Bova172_5265 | | | | | | | | | | | | | |
| Bova172_4528 | −0.02 | 1.0E+00 | x | | | x | | x | x | x | x | x | x |
| Bova172_5193 | −1.19 | 2.3E−03 | x | x | x | x | x | x | x | x | x | x | x |
| Bova172_1971 | 0.01 | 1.0E+00 | x | | x | x | x | x | x | x | x | x | x |
| Bova172_5293 | | | | | | | | | | | | | |
| Bova172_5447 | 0.02 | 1.0E+00 | x | x | x | x | x | x | x | x | x | x | x |
| Bova172_1664 | | | | | | x | | | | x | x | x | |
| Bova172_3000 | 0.12 | 1.0E+00 | x | | x | x | | | x | | x | x | x |
| Bova172_590 | −0.01 | 1.0E+00 | x | x | x | x | | x | x | x | x | x | x |
| Bova172_5421 | 0.01 | 1.0E+00 | x | | x | x | | x | x | x | x | x | x |
| Bova172_4939 | 0.05 | 1.0E+00 | x | | x | x | x | x | x | x | x | x | x |
| Bova172_420 | | | | | | | x | | | | | | |
| Bova172_643 | −0.01 | 1.0E+00 | x | x | x | x | x | x | x | x | x | x | x |
| Bova172_1996 | −0.06 | 8.5E−01 | x | | x | x | x | x | x | x | x | x | x |
| Bova172_3984 | | | | | | x | | | | | | | |
| Bova172_545 | | | | x | | x | x | | x | | x | x | x |
| Bova172_1224 | | | | | | | | x | x | x | x | | x |
| Bova172_5291 | | | | | | | | | | | | x | |
| Bova172_2884 | | | | x | | x | x | x | x | x | x | x | x |
| Bova172_1407 | 0.40 | 1.6E−05 | x | x | x | x | x | x | x | x | x | x | x |
| Bova172_232 | 0.02 | 1.0E+00 | x | x | x | x | x | x | x | x | x | x | x |
| Bova172_5094 | | | | | | | | x | | | | | |
| Bova172_66 | 0.04 | 7.8E−01 | x | x | x | x | x | x | x | x | x | x | x |
| Bova172_1976 | 0.02 | | | | | | | x | | x | x | x | x |
| Bova172_5092 | | | | | | | | x | | | | | |
| Bova172_665 | −0.07 | 1.0E+00 | x | x | x | x | x | x | x | x | x | x | x |
| Bova172_2537 | | | | x | x | | x | x | | x | x | x | x |
| Bova172_4940 | −0.05 | 1.0E+00 | x | | x | x | x | x | x | x | x | x | x |
| Bova172_3245 | 0.03 | 1.0E+00 | x | x | x | x | x | x | x | x | x | x | x |
| Bova172_1227 | | | | | | | | x | x | x | x | | x |
| Bova172_3817 | −0.11 | 6.0E−01 | x | x | | x | x | x | | x | x | x | x |
| Bova172_1230 | 0.00 | 1.0E+00 | x | x | x | x | x | x | x | x | x | x | x |
| Bova172_4274 | | | | | | | | | | | | | |
| Bova172_1645 | 0.02 | 1.0E+00 | x | | | | | | | x | x | x | |
| Bova172_5442 | 0.01 | 1.0E+00 | x | | x | x | x | x | x | x | x | x | x |
| Bova172_5579 | 0.20 | 3.1E−01 | x | | x | | x | x | x | | | | x |
| Bova172_4162 | 0.20 | 2.8E−01 | x | x | x | x | x | x | x | x | x | x | x |
| Bova172_1090 | | | | | | x | | x | x | x | x | x | x |
| Bova172_4145 | −0.03 | 9.0E−01 | x | | | | | x | | x | x | x | x |
| Bova172_561 | −0.03 | 1.0E+00 | x | x | x | x | | x | x | | | | x |
| Bova172_5605 | | | | | | | | | | | | | |
| Bova172_3118 | | | | | | | x | x | | x | x | x | x |
| Bova172_3829 | −0.09 | 7.1E−01 | x | | x | x | x | | | x | x | x | x |
| Bova172_2536 | | | | x | x | | x | x | | x | x | x | x |
| Bova172_4293 | −0.39 | 2.6E−03 | x | x | x | x | x | x | x | x | x | x | x |
| Bova172_1954 | −0.21 | 2.8E−01 | x | | x | | x | x | | x | x | x | x |
| Bova172_2613 | | | | x | x | x | | x | x | x | x | x | x |
| Bova172_1253 | | | | | | | | | | | | | |
| Bova172_3738 | | | | | | | | | | | | | |
| Bova172_1300 | −0.02 | 1.0E+00 | x | x | x | x | | x | x | x | x | x | x |
| Bova172_2535 | | | | x | x | | x | x | | x | x | x | x |
| Bova172_1228 | | | | | | | | x | x | x | x | | x |
| Bova172_3860 | 0.11 | 1.0E+00 | x | | | | x | x | x | x | x | x | x |
| Bova172_387 | | | | | | | x | | | | | | |
| Bova172_5394 | −0.03 | 1.0E+00 | x | | x | x | x | x | | x | x | x | x |
| Bova172_845 | | | | | | | | | | | x | x | |
| Bova172_2802 | −0.03 | 1.0E+00 | x | x | x | x | x | x | x | x | x | x | x |
| Bova172_2696 | | | | | | | | x | | x | x | | x |
| Bova172_3862 | 0.00 | 1.0E+00 | x | x | | | x | x | x | x | x | x | x |
| Bova172_1110 | | | | | | x | | | | | | | x |
| Bova172_765 | | | | | | | | | | x | x | x | x |
| Bova172_5202 | 0.01 | 1.0E+00 | x | | | | x | x | x | x | x | x | x |
| Bova172_358 | 0.00 | 1.0E+00 | x | | x | x | x | x | x | x | x | x | x |
| Bova172_2955 | −0.01 | 1.0E+00 | x | | x | | | x | x | | x | x | x |
| Bova172_4067 | | | | | | | | | | | | | |
| Bova172_1981 | | | | | | | | x | | x | x | x | x |
| Bova172_1417 | −0.07 | 8.1E−01 | x | x | x | x | x | x | x | x | x | x | x |
| Bova172_5405 | 0.01 | 1.0E+00 | x | | x | | | x | x | x | x | x | x |
| Bova172_1313 | 0.01 | 1.0E+00 | x | | x | x | | x | x | x | x | x | x |
| Bova172_465 | 0.01 | 1.0E+00 | x | | | | x | | | | | | |
| Bova172_2464 | | | | x | x | x | | x | x | x | x | x | x |
| Bova172_3192 | −0.05 | 1.0E+00 | x | x | x | x | x | x | x | x | x | x | x |
| Bova172_1517 | 0.08 | 7.1E−01 | x | x | x | x | x | x | x | x | x | x | x |
| Bova172_2081 | 0.02 | 9.0E−01 | x | x | x | x | | x | x | x | x | x | x |
| Bova172_1952 | 0.07 | 1.0E+00 | x | | x | | | x | | x | x | x | x |
| Bova172_4599 | | | | | | | | x | | x | x | x | x |
| Bova172_3870 | | | | | | | x | x | | x | x | | x |
| Bova172_1271 | 0.01 | 1.0E+00 | x | | x | x | x | x | x | x | x | x | x |
| Bova172_1980 | | | | | | | | x | | x | x | x | x |
| Bova172_4546 | −0.01 | 1.0E+00 | x | | x | x | x | x | x | x | x | x | x |
| Bova172_4934 | −0.07 | 7.1E−01 | x | | x | x | x | x | x | x | x | x | x |
| Bova172_4585 | | | | | | | | x | | x | x | x | x |
| Bova172_4658 | 0.02 | 1.0E+00 | x | x | x | x | | x | x | x | x | x | x |
| Bova172_1695 | | | | | | x | | | | x | x | x | |
| Bova172_5629 | | | | | | | | | | | | | |
| Bova172_2886 | | | | x | | x | x | x | x | x | x | x | x |
| Bova172_2999 | −0.02 | 9.4E−01 | x | | x | x | | | x | | x | x | x |
| Bova172_3116 | 0.01 | 1.0E+00 | x | x | x | x | x | x | x | x | x | x | x |
| Bova172_2720 | −0.04 | 1.0E+00 | x | x | x | x | | x | x | x | | x | |
| Bova172_2982 | 0.03 | 1.0E+00 | x | | x | x | | x | | x | x | x | x |
| Bova172_2498 | 0.05 | 1.0E+00 | x | x | x | x | | x | x | x | x | x | x |
| Bova172_4101 | 0.03 | 9.0E−01 | x | | x | x | x | x | x | x | x | x | x |
| Bova172_1113 | | | | | | x | | | | | | | x |
| Bova172_4079 | | | | x | x | x | | x | | x | x | x | x |
| Bova172_2885 | | | | x | | x | x | x | x | x | x | x | x |
| Bova172_3987 | | | | | | x | | | | | | | |
| Bova172_2534 | | | | x | x | | x | x | | x | x | x | x |
| Bova172_4436 | | | | x | x | | x | x | | x | | | x |
| Bova172_4958 | −0.06 | 1.0E+00 | x | x | x | x | x | x | x | x | x | x | x |
| Bova172_4839 | −0.70 | 3.1E−22 | x | x | x | x | x | x | x | x | x | x | x |
| Bova172_2469 | | | | x | x | x | x | x | x | x | x | x | x |
| Bova172_4787 | | | | | | | | | | x | x | x | x |
| Bova172_453 | | | | | | | | | | | | | |
| Bova172_3196 | | | | x | x | x | | | | | | | x |

Orthologs (x = present, >97% similarity) of B. ovatus 17.2 genes involved in N-methylserotonin release in the other non-releasing B. ovatus strains screened

TABLE S6B
Functional predictions of CAZymes deemed to be candidate mediators of N-methylserotonin release.

| | | Best hit against | | Reported activity | Predicted |
|---|---|---|---|---|---|
| | CAZy | experimentally | % identity | in literature | function |
| Gene | Annotation | characterized enzymes | to hit | (PMID) | — |
| Bova172_370 | PL1_2 | Pectate lyase | 61 | 28329766 | Pectate lyase |
| | | (Bacteroides | | | |
| | | thetaiotaomicron VPI-5482) | | | |
| Bova172_372 | GH2 | α--arabinopyranosidase | 90 | 28329766 | α--arabinopyranosidase |
| | | (Bacteroides | | | |
| | | thetaiotaomicron VPI-5482) | | | |
| Bova172_373 | GH139 | α--2-O—Me—L- | 80 | 28329766 | α--2-O—Me—L- |
| | | fucosidase (Bacteroides | | | fucosidase |
| | | thetaiotaomicron VPI-5482) | | | |
| Bova172_375 | GH106 | α-rhamnosidase | 84 | 28329766 | α-rhamnosidase |
| | | (Bacteroides | | | |
| | | thetaiotaomicron VPI-5482) | | | |
| Bova172_539 | GH137-GH2 | bimodular β-L- | 90 | 28329766 | bimodular β-L- |
| | | arabinofuranosidase/β- | | | arabinofuranosidase/β- |
| | | glucuronidase (3.2.1.31; | | | glucuronidase |
| | | 3.2.1.185) Bacteroides | | | |
| | | thetaiotaomicron VPI-5482 | | | |
| Bova172_540 | GH138 | rhamnogalacturonan α-1,2- | 95 | 28329766 | rhamnogalacturonan α- |
| | | galacturonohydrolase | | | 1,2-galacturonohydrolase |
| | | (3.2.1.173) Bacteroides | | | |
| | | thetaiotaomicron VPI-5482 | | | |
| Bova172_588 | GH95 | α-galactosidase | 92 | 28329766 | α--galactosidase |
| | | (Bacteroides | | | |
| | | thetaiotaomicron VPI-5482) | | | |
| Bova172_589 | GH105 | RG-II backbone hydrolase | 92 | 28329766 | RG-II backbone |
| | | (Bacteroides | | | hydrolase |
| | | thetaiotaomicron VPI-5482) | | | |
| Bova172_590 | GH140 | Endo-apiosidase | 95 | 28329766 | Endo-apiosidase |
| | | (Bacteroides | | | |
| | | thetaiotaomicron VPI-5482) | | | |
| Bova172_591 | GH78-GH33 | α-rhamnosidase/Kdo- | 87 | 28329766 | α-rhamnosidase/Kdo- |
| | | hydrolase (Bacteroides | | | hydrolase |
| | | thetaiotaomicron VPI-5482) | | | |
| Bova172_1037 | GH31 | α-glucosidase | 91 | 23036359 | α-glucosidase |
| | | (Bacteroides | | | |
| | | thetaiotaomicron VPI-5482) | | | |
| Bova172_1088 | GH28 | α-D-galacturonidase | 78 | 29255254 | α-D-galacturonidase |
| | | (Bacteroides | | | |
| | | thetaiotaomicron VPI-5482) | | | |
| Bova172_1090 | GH105 | Unsaturated | 61 | 28637865 | Unsaturated |
| | | rhamnogalacturonyl | | | rhamnogalacturonyl |
| | | hydrolase (Bacteroides | | | hydrolase |
| | | thetaiotaomicron VPI-5482) | | | |
| Bova172_1117 | GH43_24 | Exo-β-(1-3)- | 85 | 29255254 | Exo-β-(1-3)- |
| | | galactosidase | | | galactosidase |
| | | (Bacteroides | | | |
| | | thetaiotaomicron VPI-5482) | | | |
| Bova172_1118 | GH43_24 | Endo- β-(1-3)- | 82 | 29255254 | Endo- β-(1- |
| | | galactanase | | | 3)-galactanase |
| | | (Bacteroides | | | |
| | | thetaiotaomicron VPI-5482) | | | |
| Bova172_1119 | PL27 | Rhamno-glucurono | 65 | 28637865 | Rhamno-glucurono |
| | | lyase (Bacteroides | | | lyase |
| | | cellulosilyticus | | | |
| | | DSM 14838) | | | |
| Bova172_1222 | GH88 | d-4,5-unsaturated β- | 42 | 12044176 | unsaturated |
| | | glucuronidase/unsaturated | | | glucuronyl |
| | | glucuronyl hydrolase | | | hydrolase |
| | | (3.2.1.—) Pedobacter | | | |
| | | heparinus DSM 2366 | | | |
| Bova172_1223 | GH43_10 | β-xylosidase (3.2.1.37) | 41 | 15056894 | α-L/β-D- |
| | | Thermoclostridium | | | glycosidase |
| | | stercorarium F-9 | | | |
| Bova172_1224 | GH36 | galactomannan-specific | 75 | 27288925 | α-galactosidase |
| | | α-galactosidase | | | |
| | | (3.2.1.22) Bacteroides | | | |
| | | ovatus ATCC 8483 | | | |
| Bova172_1932 | GH105 | unsaturated | 73 | 29255254 | unsaturated |
| | | rhamnogalacturonyl | | | glucuronyl |
| | | hydrolase (3.2.1.172) | | | hydrolase |
| | | Bacteroides | | | |
| | | thetaiotaomicron VPI-5482 | | | |
| Bova172_1935 | GH28 | polygalacturonase | 45 | 22711301 | possible β- |
| | | (3.2.1.15) uncultured | | | polygalacturonase |
| | | bacterium | | | |
| Bova172_1946 | PL1 | pectate lyase (4.2.2.2) | 30 | 29255254 | polysaccharide |
| | | Bacteroides | | | lyase |
| | | thetaiotaomicron VPI-5482 | | | |
| Bova172_1948 | GH105 | d-4,5-unsaturated α- | 51 | 28329766 | unsaturated |
| | | galacturonidase cleaving | | | glucuronyl |
| | | d-4,5-unsaturated-α- | | | hydrolase |
| | | Galp-α-1,4-D-Galp) | | | |
| | | (3.2.1.—) Bacteroides | | | |
| | | thetaiotaomicron VPI-5482 | | | |
| Bova172_1949 | GH28 | RGI-specific α- | 67 | 29255254 | α-galacturonidase |
| | | galacturonidase (3.2.1.—) | | | |
| | | Bacteroides | | | |
| | | thetaiotaomicron VPI-5482 | | | |
| Bova172_1952 | PL9 | rhamnogalacturonan | 71 | 29255254 | rhamnogalacturonan |
| | | lyase (4.2.2.23) | | | lyase |
| | | Bacteroides | | | |
| | | thetaiotaomicron VPI-5482 | | | |
| Bova172_1953 | GH28 | RGI specific α- | 69 | 29255254 | α-galacturonidase |
| | | galacturonidase (3.2.1.—) | | | |
| | | Bacteroides | | | |
| | | thetaiotaomicron VPI-5482 | | | |
| Bova172_1954 | PL11 | rhamnogalacturonan lyase | 71 | 29255254 | rhamnogalacturonan |
| | | (4.2.2.23) Bacteroides | | | lyase |
| | | thetaiotaomicron VPI-5482 | | | |
| Bova172_1956 | GH2 | β-glucuronidase | 82 | 29255254 | β-glucuronidase |
| | | (3.2.1.31) Bacteroides | | | |
| | | thetaiotaomicron VPI-5482 | | | |
| Bova172_1968 | GH105 | unsaturated | 84 | 29255254 | unsaturated |
| | | rhamnogalacturonyl | | | rhamnogalacturonyl |
| | | hydrolase | | | hydrolase |
| | | (3.2.1.172) Bacteroides | | | |
| | | thetaiotaomicron VPI-5482 | | | |
| Bova172_1972 | GH105 | unsaturated | 90 | 29255254 | unsaturated |
| | | rhamnogalacturonyl | | | rhamnogalacturonyl |
| | | hydrolase | | | hydrolase/RGI- |
| | | (3.2.1.172)/RGI- | | | disaccharide |
| | | disaccharide specific | | | specific unsaturated |
| | | unsaturated | | | galacturonidase |
| | | galacturonidase | | | |
| | | Bacteroides | | | |
| | | thetaiotaomicron VPI-5482 | | | |
| Bova172_1973 | PL11 | rhamnogalacturonan | 86 | 29255254 | rhamnogalacturonan |
| | | lyase (4.2.2.23) | | | lyase |
| | | Bacteroides | | | |
| | | thetaiotaomicron VPI-5482 | | | |
| Bova172_1974 | GH105 | unsaturated | 92 | 29255254 | unsaturated |
| | | rhamnogalacturonyl | | | rhamnogalacturonyl |
| | | hydrolase (3.2.1.172)/RGI- | | | hydrolase/RGI-specific |
| | | specific unsaturated | | | unsaturated |
| | | galacturonidase | | | galacturonidase |
| | | Bacteroides | | | |
| | | thetaiotaomicron VPI-5482 | | | |
| Bova172_1976 | PL26 | rhamnogalacturonan | 35 | 25921806 | polysaccharide |
| | | lyase (4.2.2.24) | | | lyase |
| | | Penicillium | | | |
| | | chrysogenum 31B | | | |
| Bova172_1977 | PL11 | rhamnogalacturonan | 37 | 22112403 | polysaccharide |
| | | lyase (4.2.2.23) | | | lyase |
| | | Bacillus | | | |
| | | licheniformis | | | |
| | | DSM 13 = | | | |
| | | ATCC 14580 | | | |
| Bova172_1990 | GH28 | homogalacturonan | 88 | 29255254 | homogalacturonan |
| | | specific α- | | | specific α- |
| | | galacturonidase/exo- | | | galacturonidase/exo- |
| | | polygalacturonase | | | polygalacturonase |
| | | Bacteroides | | | |
| | | thetaiotaomicron VPI-5482 | | | |
| Bova172_1992 | GH42 | B-galactosidase | 31 | doi.org/10.1016/j.foodchem.2010.08.075 | β-D-glycosidase |
| | | (3.2.1.23) Thermotoga | | | |
| | | maritima MSB8 | | | |
| Bova172_1993 | GH43_18 | α-L-arabinofuranosidase | 85 | 30850540 | α-L/β-D- |
| | | (weak activity) | | | glycosidase |
| | | Bacteroides | | | |
| | | cellulosilyticus WH2 | | | |
| Bova172_1994 | GH2 | β-galactosidase | 89 | 29255254 | β-galactosidase |
| | | (3.2.1.23) Bacteroides | | | |
| | | thetaiotaomicron VPI-5482 | | | |
| Bova172_1996 | GH28 | RGI specific α- | 89 | 29255254 | RGI specific α- |
| | | galacturonidase (3.2.1.—) | | | galacturonidase |
| | | Bacteroides | | | |
| | | thetaiotaomicron VPI-5482 | | | |
| Bova172_1998 | GH28 | RGI-disaccharide | 93 | 29255254 | RGI-disaccharide |
| | | specific α-galacturonidase | | | specific α- |
| | | (3.2.1.—) Bacteroides | | | galacturonidase |
| | | thetaiotaomicron VPI-5482 | | | |
| Bova172_1999 | GH106 | rhamnogalacturonan α- | 89 | 29255254 | rhamnogalacturonan α- |
| | | L-rhamnohydrolase | | | L-rhamnohydrolase/RGI |
| | | (3.2.1.174)/RGI specific | | | specific alpha-L- |
| | | alpha-L-rhamnosidase | | | rhamnosidase |
| | | Bacteroides | | | |
| | | thetaiotaomicron VPI-5482 | | | |
| Bova172_2080 | GH92 | α-(1-4)-mannosidase | 86 | 20081828 | α-(1-4)- |
| | | (Bacteroides | | | mannosidase |
| | | thetaiotaomicron VPI-5482) | | | |
| Bova172_2470 | GH95 | α-galactosidase | 52 | 28329766 | α-galactosidase |
| | | (Bacteroides | | | |
| | | thetaiotaomicron VPI-5482) | | | |
| Bova172_2533 | GH13 | α-amylase | 90 | 8955399 | α-amylase |
| | | (Neopullulanase) | | | (Neopullulanase) |
| | | SusA (Bacteroides | | | |
| | | thetaiotaomicron VPI-5482) | | | |
| Bova172_2534 | GH97 | Glucan 1,4-α- | 93 | 18981178 | Glucan 1,4-α- |
| | | glucosidase SusB | | | glucosidase |
| | | (Bacteroides | | | |
| | | thetaiotaomicron VPI-5482) | | | |
| Bova172_3245 | GH3 | β-glucosidase | 99 | 29020628 | β-glucosidase |
| | | (Bacteroides | | | |
| | | ovatus ATCC 8483) | | | |
| Bova172_3249 | GH16 | β-(1-3)-endoglucanase | 100 | 29020628 | β-(1-3)- |
| | | (Bacteroides | | | endoglucanase |
| | | ovatus ATCC 8483) | | | |

Table S7A. B. ovatus TSDC 17.2

| Expression | | |
|---|---|---|
| TYG + hemin, ±OF | TYG − hemin, ±OF | MEGA, ±OF |

| Gene (in strain 17.2) | PUL | RAST Annotation | CAZy Classification | Log2fold | Padj | Log2fold | Padj | Log2fold | Padj |
|---|---|---|---|---|---|---|---|---|---|
| Bova172_139 | Predicted | RNA polymerase ECF-type sigma | REG | −0.70 | 3.4E−04 | −0.25 | 1.8E−01 | 0.41 | 3.0E−01 |
| | PUL 1 | factor | | | | | | | |
| Bova172_140 | Predicted | Putative anti-sigma factor | REG | 0.17 | 9.5E−02 | −2.80 | 3.4E−99 | 0.06 | 8.0E−01 |
| | PUL 1 | | | | | | | | |
| Bova172_141 | Predicted | Outer membrane TonB-dependent | Trans | 0.58 | 1.1E−07 | −2.42 | 1.7E−101 | −0.14 | 4.8E−01 |
| | PUL 1 | transporter, SusC family | | | | | | | |
| Bova172_142 | Predicted | Cell surface glycan-binding | SusD | 1.06 | 9.7E−18 | −2.16 | 4.2E−64 | 0.95 | 3.4E−04 |
| | PUL 1 | lipoprotein, SusD family | | | | | | | |
| Bova172_143 | Predicted | Arylsulfatase (EC 3.1.6.1) | Sulf | −0.36 | 6.6E−02 | −1.44 | 2.3E−18 | −0.56 | 1.7E−01 |
| | PUL 1 | | | | | | | | |
| Bova172_144 | Predicted | beta-galactosidase (EC 3.2.1.23) | GH2 | 0.15 | 3.2E−01 | −0.56 | 4.1E−07 | −0.60 | 4.3E−02 |
| | PUL 1 | | | | | | | | |
| Bova172_370 | CAZyme | Pectate lyase (EC 4.2.2.2) | PL1_2 | 1.72 | 5.3E−30 | −0.34 | 1.0E−02 | 2.15 | 2.2E−25 |
| | cluster 1 | | | | | | | | |
| Bova172_371 | CAZyme | BaeS-type histidine kinase/ | HTCS_Rgu-3 | −1.47 | 1.6E−106 | −0.78 | 1.3E−39 | −0.80 | 3.6E−12 |
| | cluster 1 | OmpR-type DNA-binding response | | | | | | | |
| | | regulator | | | | | | | |
| Bova172_372 | CAZyme | α--arabinopyranosidase | GH2 | 0.23 | 4.6E−01 | 0.46 | 8.4E−02 | −0.06 | 9.1E−01 |
| | cluster 1 | | | | | | | | |
| Bova172_373 | CAZyme | α--2-O-Me-L-fucosidase | GH139 | 3.03 | 3.6E−06 | 1.16 | 1.1E−02 | 0.42 | 4.1E−01 |
| | cluster 1 | | | | | | | | |
| Bova172_{—}374 | CAZyme | Sialic acid-specific 9-O- | | 2.69 | 3.9E−07 | 0.34 | 3.9E−01 | 0.37 | 4.6E−01 |
| | cluster 1 | acetylesterase | | | | | | | |
| Bova172_375 | CAZyme | α-rhamnosidase | GH106 | 2.84 | 1.6E−21 | 0.72 | 9.0E−04 | 0.94 | 3.7E−02 |
| | cluster 1 | | | | | | | | |
| Bova172_539 | Predicted | bimodular β-L-arabinofuranosidase/ | GH137- | 2.29 | 1.1E−09 | 0.79 | 9.2E−03 | 1.27 | 1.2E−02 |
| | PUL 2 | β-glucuronidase | CBM57-GH2 | | | | | | |
| Bova172_540 | Predicted | rhamnogalacturonan α-1,2- | GH138 | 2.08 | 1.9E−10 | 0.05 | 8.7E−01 | 1.77 | 1.3E−04 |
| | PUL 2 | galacturonohydrolase | | | | | | | |
| Bova172_541 | Predicted | Two-component system sensor | | −0.44 | 1.4E−05 | 1.47 | 3.0E−68 | −0.24 | 4.1E−01 |
| | PUL 2 | histidine kinase | | | | | | | |
| Bova172_542 | Predicted | hypothetical protein | | 1.08 | 1.7E−02 | −0.40 | 1.6E−01 | 0.46 | 3.2E−01 |
| | PUL 2 | | | | | | | | |
| Bova172_543 | Predicted | hypothetical protein | | 2.00 | 8.1E−06 | −0.32 | 1.3E−01 | −0.23 | 6.4E−01 |
| | PUL 2 | | | | | | | | |
| Bova172_544 | Predicted | endonuclease/exonuclease/ | | 0.96 | 5.3E−02 | −0.14 | 6.9E−01 | −0.40 | 4.2E−01 |
| | PUL 2 | phosphatase family protein | | | | | | | |
| Bova172_{—}545 | Predicted | Cell surface glycan-binding | SusD | 2.00 | 2.7E−05 | −0.20 | 4.3E−01 | −0.07 | 8.8E−01 |
| | PUL 2 | lipoprotein, SusD family | | | | | | | |
| Bova172_{—}546 | Predicted | Outer membrane TonB-dependent | Trans | 2.71 | 5.8E−17 | −0.65 | 1.7E−05 | 0.38 | 3.4E−01 |
| | PUL 2 | transporter, SusC family | | | | | | | |
| Bova172_547 | Predicted | hypothetical protein | REG | 0.94 | 1.8E−12 | −0.71 | 8.0E−09 | 0.25 | 3.2E−01 |
| | PUL 2 | | | | | | | | |
| Bova172_548 | Predicted | RNA polymerase ECF-type sigma | REG | −0.66 | 4.5E−07 | −0.86 | 2.4E−15 | 0.65 | 9.5E−04 |
| | PUL 2 | factor | | | | | | | |
| Bova172_{—}561 | Predicted | Cytochrome c-type biogenesis | | 1.64 | 8.2E−07 | −2.19 | 9.2E−21 | 0.24 | 5.8E−01 |
| | PUL 3 | protein DsbD, protein-disulfide | | | | | | | |
| | | reductase (EC 1.8.1.8) | | | | | | | |
| Bova172_562 | Predicted | hypothetical protein | | 0.18 | 7.6E−01 | −1.08 | 7.5E−02 | −0.07 | 1.0E+00 |
| | PUL 3 | | | | | | | | |
| Bova172_563 | Predicted | hypothetical protein | Pept | 0.01 | 9.8E−01 | −0.94 | 1.6E−02 | −0.33 | 4.8E−01 |
| | PUL 3 | | | | | | | | |
| Bova172_564 | Predicted | hypothetical protein | | −0.25 | 5.6E−01 | −0.23 | 5.9E−01 | 0.04 | 9.5E−01 |
| | PUL 3 | | | | | | | | |
| Bova172_565 | Predicted | hypothetical protein | | 0.96 | 6.3E−02 | −1.09 | 1.1E−02 | 0.27 | 5.9E−01 |
| | PUL 3 | | | | | | | | |
| Bova172_566 | Predicted | hypothetical protein | | −0.58 | 3.2E−01 | −0.43 | 4.3E−01 | 0.03 | 1.0E+00 |
| | PUL 3 | | | | | | | | |
| Bova172_567 | Predicted | hypothetical protein | | −0.41 | 4.4E−01 | −0.69 | 1.6E−01 | −0.07 | 1.0E+00 |
| | PUL 3 | | | | | | | | |
| Bova172_568 | Predicted | hypothetical protein | | −0.27 | 6.3E−01 | −0.64 | 2.3E−01 | 0.13 | 1.0E+00 |
| | PUL 3 | | | | | | | | |
| Bova172_569 | Predicted | hypothetical protein | | 0.02 | 9.8E−01 | −0.52 | 3.5E−01 | 0.13 | 1.0E+00 |
| | PUL 3 | | | | | | | | |
| Bova172_570 | Predicted | hypothetical protein | | −0.70 | 2.4E−01 | −1.34 | 4.5E−02 | 0.14 | 1.0E+00 |
| | PUL 3 | | | | | | | | |
| Bova172_571 | Predicted | hypothetical protein | | −0.17 | 7.6E−01 | −0.51 | 3.9E−01 | −0.26 | 1.0E+00 |
| | PUL 3 | | | | | | | | |
| Bova172_572 | Predicted | hypothetical protein | SusD | −0.09 | 8.7E−01 | −0.59 | 2.9E−01 | 0.25 | 1.0E+00 |
| | PUL 3 | | | | | | | | |
| Bova172_573 | Predicted | Outer membrane TonB-dependent | Trans | 0.37 | 4.7E−01 | −0.82 | 8.3E−02 | −0.20 | 1.0E+00 |
| | PUL 3 | transporter, SusC family | | | | | | | |
| Bova172_588 | Predicted | α--galactosidase | GH95 | 2.61 | 4.6E−13 | −1.24 | 2.1E−05 | 1.24 | 1.7E−02 |
| | PUL 4 | | | | | | | | |
| Bova172_{—}589 | Predicted | RG-II backbone hydrolase | GH105 | 2.39 | 3.4E−08 | −0.36 | 3.6E−01 | 0.36 | 4.7E−01 |
| | PUL 4 | | | | | | | | |
| Bova172_{—}590 | Predicted | Endo-apiosidase | GH140 | 2.15 | 1.1E−07 | −0.11 | 7.7E−01 | 0.65 | 2.1E−01 |
| | PUL 4 | | | | | | | | |
| Bova172_591 | Predicted | α-rhamnosidase/Kdo-hydrolase | GH78-GH33 | 3.86 | 1.6E−16 | 1.24 | 6.3E−04 | 0.11 | 8.4E−01 |
| | PUL 4 | | | | | | | | |
| Bova172_592 | Predicted | Oxidoreductase, short-chain | | 1.33 | 1.9E−34 | −1.94 | 8.0E−72 | 1.09 | 6.7E−21 |
| | PUL 4 | dehydrogenase/reductase family | | | | | | | |
| Bova172_593 | Predicted | Serine protease | | 0.98 | 4.7E−17 | −1.83 | 1.8E−59 | 1.68 | 3.2E−30 |
| | PUL 4 | | | | | | | | |
| Bova172_594 | Predicted | hypothetical protein | CE | 0.23 | 2.5E−01 | 0.77 | 1.2E−06 | −0.28 | 4.6E−01 |
| | PUL 4 | | | | | | | | |
| Bova172_595 | Predicted | Polygalacturonase (EC 3.2.1.15) | GH28 | −0.12 | 5.6E−01 | −0.43 | 4.0E−03 | 0.75 | 4.7E−02 |
| | PUL 4 | | | | | | | | |
| Bova172_596 | Predicted | putative secreted hydrolase | GH78 | 2.80 | 1.2E−07 | 1.20 | 5.6E−03 | 0.57 | 2.6E−01 |
| | PUL 4 | | | | | | | | |
| Bova172_597 | Predicted | hypothetical protein | GH142- | 2.55 | 3.8E−18 | 0.25 | 3.1E−01 | 0.44 | 2.3E−01 |
| | PUL 4 | | GH143 | | | | | | |
| Bova172_598 | Predicted | arabinosidase | GH43_18 | 1.39 | 1.8E−08 | −2.12 | 5.3E−17 | 0.54 | 5.4E−02 |
| | PUL 4 | | | | | | | | |
| Bova172_599 | Predicted | hypothetical protein | | 1.57 | 9.3E−22 | −0.53 | 3.5E−03 | 0.85 | 1.8E−03 |
| | PUL 4 | | | | | | | | |
| Bova172_600 | Predicted | Pectate lyase (EC 4.2.2.2) | PL1_2 | 0.80 | 7.5E−08 | −0.51 | 5.3E−04 | 0.13 | 6.1E−01 |
| | PUL 4 | | | | | | | | |
| Bova172_601 | Predicted | Cell surface glycan-binding | SusD | 4.14 | 1.1E−42 | 0.66 | 4.5E−03 | 0.53 | 1.6E−01 |
| | PUL 4 | lipoprotein, SusD family | | | | | | | |
| Bova172_602 | Predicted | Outer membrane TonB-dependent | Trans | 4.04 | 6.4E−45 | −0.07 | 7.6E−01 | 1.51 | 3.5E−05 |
| | PUL 4 | transporter, SusC family | | | | | | | |
| Bova172_603 | Predicted | hypothetical protein | | 3.62 | 7.5E−36 | −0.21 | 3.3E−01 | 1.43 | 1.2E−04 |
| | PUL 4 | | | | | | | | |
| Bova172_604 | Predicted | hypothetical protein | | 1.30 | 1.4E−02 | −0.16 | 7.8E−01 | 0.34 | 1.0E+00 |
| | PUL 4 | | | | | | | | |
| Bova172_605 | Predicted | ISNCY family transposase | | −0.13 | 8.0E−01 | −1.95 | 3.8E−04 | 1.09 | 3.8E−02 |
| | PUL 4 | | | | | | | | |
| Bova172_606 | Predicted | Alpha-1,2-mannosidase | GH92 | −1.77 | 1.3E−100 | −1.90 | 1.1E−135 | −2.70 | 2.9E−63 |
| | PUL 4 | | | | | | | | |
| Bova172_607 | Predicted | 1,4-beta-mannosyl-N- | GH130 | −1.97 | 1.5E−74 | −2.19 | 9.9E−117 | −2.34 | 2.4E−27 |
| | PUL 4 | acetylglucosamine phosphorylase | | | | | | | |
| | | (EC 2.4.1.320) | | | | | | | |
| Bova172_608 | Predicted | AmpG protein, beta-lactamase | Trans | −1.22 | 1.2E−28 | −2.93 | 1.6E−171 | −1.28 | 1.4E−13 |
| | PUL 4 | induction signal transducer | | | | | | | |
| Bova172_609 | Predicted | beta-hexosaminidase precursor | GH163 | −0.19 | 2.6E−01 | −2.01 | 5.3E−69 | −1.69 | 4.1E−07 |
| | PUL 4 | | | | | | | | |
| Bova172_610 | Predicted | hypothetical protein | | −0.67 | 8.2E−05 | −2.42 | 1.3E−53 | −1.74 | 1.8E−13 |
| | PUL 4 | | | | | | | | |
| Bova172_611 | Predicted | Patatin-like protein | | −0.63 | 1.5E−04 | −2.29 | 1.2E−51 | −2.53 | 2.6E−24 |
| | PUL 4 | | | | | | | | |
| Bova172_612 | Predicted | Endo-beta-N- | GH18 | −0.74 | 6.2E−09 | −2.18 | 2.3E−83 | −2.56 | 1.9E−31 |
| | PUL 4 | acetylglucosaminidase F2 | | | | | | | |
| Bova172_613 | Predicted | Cell surface glycan-binding | SusD | −1.13 | 1.6E−22 | −2.28 | 2.3E−102 | −2.47 | 2.7E−39 |
| | PUL 4 | lipoprotein, SusD family | | | | | | | |
| Bova172_614 | Predicted | Outer membrane TonB-dependent | Trans | −0.97 | 1.5E−20 | −3.26 | 2.1E−224 | −1.92 | 7.6E−52 |
| | PUL 4 | transporter, SusC family | | | | | | | |
| Bova172_618 | Predicted | Outer membrane TonB-dependent | Trans | −0.08 | 5.3E−01 | −4.54 | 0.0E+00 | −2.08 | 1.4E−23 |
| | PUL 5 | transporter, SusC family | | | | | | | |
| Bova172_619 | Predicted | Cell surface glycan-binding | SusD | 0.13 | 4.8E−01 | −4.10 | 2.9E−270 | −2.04 | 5.4E−10 |
| | PUL 5 | lipoprotein, SusD family | | | | | | | |
| Bova172_620 | Predicted | Endo-beta-N- | GH18 | 0.19 | 2.8E−01 | −3.77 | 6.2E−230 | −1.61 | 2.7E−06 |
| | PUL 5 | acetylglucosaminidase F2 | | | | | | | |
| Bova172_621 | Predicted | putative patatin-like protein | | 0.83 | 1.3E−08 | −3.87 | 0.0E+00 | −2.07 | 5.6E−11 |
| | PUL 5 | | | | | | | | |
| Bova172_622 | Predicted | hypothetical protein | | 0.57 | 4.1E−04 | −3.70 | 2.1E−244 | −1.44 | 9.9E−06 |
| | PUL 5 | | | | | | | | |
| Bova172_623 | Predicted | Putative anti-sigma factor | REG | −0.87 | 1.2E−41 | −2.66 | 0.0E+00 | −0.21 | 1.3E−01 |
| | PUL 5 | | | | | | | | |
| Bova172_624 | Predicted | RNA polymerase ECF-type sigma | REG | 0.16 | 1.3E−01 | −3.02 | 1.7E−216 | 0.75 | 7.5E−10 |
| | PUL 5 | factor | | | | | | | |
| Bova172_842 | Predicted | Cell surface glycan-binding | SusD | 0.51 | 2.9E−04 | −0.58 | 5.8E−07 | 0.74 | 1.4E−03 |
| | PUL 6 | lipoprotein, SusD family | | | | | | | |
| Bova172_843 | Predicted | Outer membrane TonB-dependent | Trans | −0.59 | 6.8E−07 | −0.61 | 6.9E−09 | 0.91 | 2.8E−07 |
| | PUL 6 | transporter, SusC family | | | | | | | |
| Bova172_915 | Predicted | beta-glycosyl hydrolase | GH20 | −1.02 | 1.2E−10 | 1.65 | 6.7E−34 | −0.37 | 2.2E−01 |
| | PUL 7 | | | | | | | | |
| Bova172_916 | Predicted | beta-glycosyl hydrolase | GH20 | −1.58 | 6.2E−36 | 0.25 | 4.4E−02 | −2.15 | 1.1E−23 |
| | PUL 7 | | | | | | | | |
| Bova172_917 | Predicted | Beta-mannosidase (EC 3.2.1.25) | GH2 | −1.14 | 3.0E−36 | −1.23 | 1.5E−45 | −0.76 | 2.1E−07 |
| | PUL 7 | | | | | | | | |
| Bova172_918 | Predicted | hypothetical protein | | −2.01 | 6.5E−10 | −0.96 | 4.0E−04 | −0.83 | 9.8E−02 |
| | PUL 7 | | | | | | | | |
| Bova172_919 | Predicted | Sialidase (EC 3.2.1.18) | GH33 | −2.09 | 2.1E−79 | −1.70 | 3.0E−60 | −0.90 | 6.2E−06 |
| | PUL 7 | | | | | | | | |
| Bova172_920 | Predicted | Uncharacterized sugar:proton | Trans | −2.02 | 1.1E−03 | −0.41 | 3.5E−01 | −0.46 | 1.0E+00 |
| | PUL 7 | symporter | | | | | | | |
| Bova172_921 | Predicted | N-acylglucosamine 2-epimerase | EPI | −1.95 | 2.2E−07 | −0.56 | 7.7E−02 | −1.01 | 3.6E−02 |
| | PUL 7 | (EC 5.1.3.8) | | | | | | | |
| Bova172_922 | Predicted | Outer membrane TonB-dependent | Trans | −1.93 | 4.0E−14 | −0.42 | 3.8E−02 | −0.88 | 2.7E−02 |
| | PUL 7 | transporter, SusC family | | | | | | | |
| Bova172_923 | Predicted | Cell surface glycan-binding | SusD | −2.51 | 3.9E−12 | −0.64 | 2.1E−02 | −1.57 | 2.9E−03 |
| | PUL 7 | lipoprotein, SusD family | | | | | | | |
| Bova172_924 | Predicted | hypothetical protein | | −1.90 | 3.5E−09 | −0.61 | 1.4E−02 | −0.64 | 1.5E−01 |
| | PUL 7 | | | | | | | | |
| Bova172_925 | Predicted | hypothetical protein | | −2.19 | 3.5E−05 | −0.11 | 7.7E−01 | 0.20 | 7.1E−01 |
| | PUL 7 | | | | | | | | |
| Bova172_926 | Predicted | S-layer related protein precursor, | GH | −0.95 | 4.1E−02 | −0.45 | 1.8E−01 | −0.47 | 3.5E−01 |
| | PUL 7 | sialic acid-specific 9-O- | | | | | | | |
| | | acetylesterase | | | | | | | |
| Bova172_927 | Predicted | hypothetical protein | | −1.37 | 7.7E−03 | 0.40 | 3.1E−01 | −0.29 | 5.5E−01 |
| | PUL 7 | | | | | | | | |
| Bova172_928 | Predicted | S-layer related protein precursor, | | −0.36 | 3.9E−01 | 0.57 | 9.8E−02 | −0.27 | 6.0E−01 |
| | PUL 7 | sialic acid-specific 9-O- | | | | | | | |
| | | acetylesterase | | | | | | | |
| Bova172_929 | Predicted | hypothetical protein | | −0.23 | 6.6E−01 | −0.25 | 5.4E−01 | 0.27 | 1.0E+00 |
| | PUL 7 | | | | | | | | |
| Bova172_930 | Predicted | alpha-galactosidase (EC 3.2.1.22) | GH27 | −0.16 | 5.0E−01 | −1.25 | 1.1E−16 | −0.76 | 3.2E−02 |
| | PUL 8 | | | | | | | | |
| Bova172_931 | Predicted | Platelet-activating factor | | −0.05 | 8.2E−01 | −1.81 | 5.3E−44 | −0.38 | 3.2E−01 |
| | PUL 8 | acetylhydrolase IB gamma subunit | | | | | | | |
| | | (EC 3.1.1.47) | | | | | | | |
| Bova172_932 | Predicted | hypothetical protein | | −0.44 | 5.5E−02 | −2.46 | 5.8E−44 | 0.15 | 7.1E−01 |
| | PUL 8 | | | | | | | | |
| Bova172_933 | Predicted | hypothetical protein | | −0.31 | 5.3E−01 | 1.11 | 1.5E−02 | 0.03 | 1.0E+00 |
| | PUL 8 | | | | | | | | |
| Bova172_934 | Predicted | Alpha-N-acetylglucosaminidase | GH89 | 0.36 | 4.7E−01 | 0.97 | 1.1E−02 | −1.20 | 3.0E−02 |
| | PUL 8 | (EC 3.2.1.50) | | | | | | | |
| Bova172_935 | Predicted | Alpha-glucosidase (EC 3.2.1.20) | GH97 | 0.64 | 1.3E−01 | 0.23 | 5.0E−01 | −0.01 | 9.8E−01 |
| | PUL 8 | | | | | | | | |
| Bova172_936 | Predicted | hypothetical protein | | −0.01 | 9.9E−01 | 0.55 | 1.8E−01 | 0.11 | 8.4E−01 |
| | PUL 8 | | | | | | | | |
| Bova172_937 | Predicted | hypothetical protein | CE | 0.15 | 7.8E−01 | 0.93 | 4.2E−02 | 0.12 | 1.0E+00 |
| | PUL 8 | | | | | | | | |
| Bova172_938 | Predicted | Alpha-glucosidase (EC 3.2.1.20) | GH97 | −0.40 | 3.7E−01 | 0.50 | 2.2E−01 | −0.21 | 6.6E−01 |
| | PUL 8 | | | | | | | | |
| Bova172_939 | Predicted | S-layer related protein precursor, | | −0.23 | 6.7E−01 | 0.27 | 5.6E−01 | −0.88 | 1.0E+00 |
| | PUL 8 | sialic acid-specific 9-O- | | | | | | | |
| | | acetylesterase | | | | | | | |
| Bova172_940 | Predicted | hypothetical protein | | −0.56 | 2.2E−01 | −0.59 | 8.6E−02 | −0.44 | 3.8E−01 |
| | PUL 8 | | | | | | | | |
| Bova172_941 | Predicted | S-layer related protein precursor, | GH | −0.41 | 3.5E−01 | −0.43 | 1.7E−01 | −0.72 | 1.2E−01 |
| | PUL 8 | sialic acid-specific 9-O- | | | | | | | |
| | | acetylesterase | | | | | | | |
| Bova172_942 | Predicted | hypothetical protein | | −0.51 | 1.2E−01 | −0.76 | 3.2E−03 | 0.04 | 9.3E−01 |
| | PUL 8 | | | | | | | | |
| Bova172_943 | Predicted | hypothetical protein | | −0.78 | 1.4E−01 | −0.71 | 1.4E−01 | −0.55 | 1.0E+00 |
| | PUL 8 | | | | | | | | |
| Bova172_944 | Predicted | Cell surface glycan-binding | SusD | −0.12 | 7.3E−01 | −1.02 | 5.0E−06 | −0.02 | 9.7E−01 |
| | PUL 8 | lipoprotein, SusD family | | | | | | | |
| Bova172_945 | Predicted | Outer membrane TonB-dependent | Trans | −1.21 | 4.0E−07 | −0.90 | 8.7E−06 | −0.25 | 5.2E−01 |
| | PUL 8 | transporter, SusC family | | | | | | | |
| Bova172_946 | Predicted | N-acylglucosamine 2-epimerase | EPI | −1.34 | 5.6E−06 | −1.37 | 1.4E−07 | 0.05 | 9.3E−01 |
| | PUL 8 | (EC 5.1.3.8) | | | | | | | |
| Bova172_947 | Predicted | Uncharacterized sugar:proton | Trans | −1.42 | 8.1E−13 | −1.94 | 3.1E−28 | −0.57 | 5.0E−02 |
| | PUL 8 | symporter | | | | | | | |
| Bova172_948 | Predicted | hypothetical protein | | −0.43 | 1.8E−02 | −2.08 | 5.4E−42 | 0.08 | 7.5E−01 |
| | PUL 8 | | | | | | | | |
| Bova172_949 | Predicted | NTP pyrophosphohydrolases | | 1.79 | 1.3E−44 | −2.49 | 9.1E−90 | 2.26 | 5.6E−72 |
| | PUL 8 | including oxidative damage repair | | | | | | | |
| | | enzymes | | | | | | | |
| Bova172_1054 | Predicted | BaeS-type histidine kinase/ | REG | −0.55 | 3.1E−04 | 3.32 | 2.9E−72 | −0.29 | 3.3E−01 |
| | PUL 9 | OmpR-type DNA-binding response | | | | | | | |
| | | regulator | | | | | | | |
| Bova172_1055 | Predicted | Glucuronyl hydrolase | GH88 | −1.42 | 4.0E−05 | 0.66 | 2.9E−02 | −0.05 | 9.3E−01 |
| | PUL 9 | | | | | | | | |
| Bova172_1056 | Predicted | beta-galactosidase (EC 3.2.1.23) | GH2 | −1.52 | 1.8E−10 | 0.68 | 1.3E−03 | −0.34 | 4.1E−01 |
| | PUL 9 | | | | | | | | |
| Bova172_1057 | Predicted | hypothetical protein | PL38 | −1.58 | 1.1E−04 | 0.55 | 9.5E−02 | −0.52 | 3.2E−01 |
| | PUL 9 | | | | | | | | |
| Bova172_1058 | Predicted | hypothetical protein | PL38 | −1.68 | 1.5E−06 | 0.89 | 3.6E−03 | −0.06 | 9.2E−01 |
| | PUL 9 | | | | | | | | |
| Bova172_1059 | Predicted | Outer membrane TonB-dependent | Trans | −1.41 | 1.1E−02 | 0.17 | 6.9E−01 | −0.36 | 1.0E+00 |
| | PUL 9 | transporter, SusC family | | | | | | | |
| Bova172_1060 | Predicted | Outer membrane TonB-dependent | Trans | −0.47 | 2.2E−01 | 2.19 | 2.8E−09 | −0.44 | 3.7E−01 |
| | PUL 9 | transporter, SusC family | | | | | | | |
| Bova172_1061 | Predicted | Cell surface glycan-binding | SusD | 0.16 | 7.7E−01 | 1.25 | 1.9E−03 | 0.14 | 1.0E+00 |
| | PUL 9 | lipoprotein, SusD family | | | | | | | |
| Bova172_1062 | Predicted | hypothetical protein | | −0.51 | 3.3E−01 | 1.45 | 1.1E−03 | −0.09 | 1.0E+00 |
| | PUL 9 | | | | | | | | |
| Bova172_1063 | Predicted | hypothetical protein | | 0.36 | 5.1E−01 | 2.58 | 8.7E−08 | −0.51 | 3.2E−01 |
| | PUL 9 | | | | | | | | |
| Bova172_1087 | CAZyme | beta-galactosidase precursor | GH35- | 0.61 | 2.5E−03 | −0.88 | 6.0E−10 | −0.28 | 4.1E−01 |
| | cluster 2 | | CBM32 | | | | | | |
| Bova172_1088 | CAZyme | α-D-galacturonidase | GH28 | 1.54 | 2.0E−04 | −1.45 | 8.1E−06 | 1.03 | 6.1E−02 |
| | cluster 2 | | | | | | | | |
| Bova172_1089 | CAZyme | hypothetical protein | GH154 | 0.49 | 1.3E−01 | −1.00 | 2.2E−04 | −0.20 | 6.8E−01 |
| | cluster 2 | | | | | | | | |
| Bova172_{—}1090 | CAZyme | Unsaturated rhamnogalacturonyl | GH105 | 1.66 | 1.1E−07 | −1.98 | 4.7E−14 | 0.41 | 3.4E−01 |
| | cluster 2 | hydrolase | | | | | | | |
| Bova172_1103 | Predicted | hypothetical protein | | 0.13 | 8.2E−01 | −1.08 | 1.6E−02 | 0.34 | 1.0E+00 |
| | PUL 10 | | | | | | | | |
| Bova172_1104 | Predicted | hypothetical protein | | 1.02 | 1.6E−02 | −0.27 | 4.0E−01 | 0.10 | 8.4E−01 |
| | PUL 10 | | | | | | | | |
| Bova172_1105 | Predicted | hypothetical protein | Pept | 0.45 | 2.7E−01 | 0.36 | 2.7E−01 | −0.16 | 7.6E−01 |
| | PUL 10 | | | | | | | | |
| Bova172_1106 | Predicted | hypothetical protein | | 0.23 | 6.1E−01 | 0.79 | 2.4E−02 | −0.45 | 3.5E−01 |
| | PUL 10 | | | | | | | | |
| Bova172_1107 | Predicted | hypothetical protein | | 0.17 | 7.5E−01 | 0.04 | 9.2E−01 | −0.55 | 2.8E−01 |
| | PUL 10 | | | | | | | | |
| Bova172_1108 | Predicted | hypothetical protein | | 0.19 | 7.1E−01 | 0.31 | 4.8E−01 | −0.42 | 1.0E+00 |
| | PUL 10 | | | | | | | | |
| Bova172_1109 | Predicted | Cell surface glycan-binding | SusD | 0.72 | 1.9E−01 | 0.02 | 9.7E−01 | 0.30 | 1.0E+00 |
| | PUL 10 | lipoprotein, SusD family | | | | | | | |
| Bova172_{—}1110 | Predicted | Outer membrane TonB-dependent | Trans | 1.42 | 2.2E−03 | 0.22 | 4.9E−01 | −0.03 | 9.6E−01 |
| | PUL 10 | transporter, SusC family | | | | | | | |
| Bova172_1111 | Predicted | hypothetical protein | | 0.98 | 8.0E−02 | −0.41 | 2.8E−01 | 0.66 | 1.0E+00 |
| | PUL 10 | | | | | | | | |
| Bova172_1112 | Predicted | hypothetical protein | | 0.32 | 5.2E−01 | 0.15 | 7.6E−01 | −0.36 | 1.0E+00 |
| | PUL 10 | | | | | | | | |
| Bova172_{—}1113 | Predicted | Cell surface glycan-binding | SusD | 1.08 | 9.7E−03 | −0.19 | 5.9E−01 | 0.85 | 1.1E−01 |
| | PUL 10 | lipoprotein, SusD family | | | | | | | |
| Bova172_1114 | Predicted | Outer membrane TonB-dependent | Trans | 1.85 | 4.7E−11 | −0.36 | 1.3E−01 | 1.57 | 6.6E−05 |
| | PUL 10 | transporter, SusC family | | | | | | | |
| Bova172_1115 | Predicted | BaeS-type histidine kinase/ | REG | −0.04 | 5.8E−01 | −0.35 | 5.5E−08 | 0.32 | 2.9E−03 |
| | PUL 10 | OmpR-type DNA-binding response | HTCS Aga-1 | | | | | | |
| | | regulator | | | | | | | |
| Bova172_1116 | Predicted | hypothetical protein | | 0.56 | 1.3E−01 | 0.08 | 8.2E−01 | 0.73 | 1.2E−01 |
| | PUL 10 | | | | | | | | |
| Bova172_1117 | Predicted | Exo-β-(1-3)-galactosidase | GH43_24 | 0.63 | 9.5E−02 | 0.11 | 7.3E−01 | 0.34 | 4.9E−01 |
| | PUL 10 | | | | | | | | |
| Bova172_1118 | Predicted | Endo-β-(1-3)-galactanase | GH43_24 | 0.62 | 1.2E−01 | 0.10 | 7.4E−01 | −0.09 | 8.7E−01 |
| | PUL 10 | | | | | | | | |
| Bova172_1119 | Predicted | Rhamno-glucurono lyase | PL27 | 0.53 | 1.8E−02 | −0.97 | 1.9E−09 | −0.85 | 2.5E−02 |
| | PUL 10 | | | | | | | | |
| Bova172_1120 | Predicted | hypothetical protein | GH43_24 | −0.13 | 6.0E−01 | −1.74 | 3.0E−16 | −0.85 | 2.5E−02 |
| | PUL 10 | | | | | | | | |
| Bova172_1154 | Predicted | hypothetical protein | | 0.42 | 3.8E−01 | 2.49 | 1.0E−09 | −0.39 | 4.4E−01 |
| | PUL 11 | | | | | | | | |
| Bova172_1155 | Predicted | hypothetical protein | | −0.52 | 3.5E−01 | 3.60 | 1.7E−09 | −0.89 | 1.1E−01 |
| | PUL 11 | | | | | | | | |
| Bova172_1156 | Predicted | hypothetical protein | Pept | 0.34 | 4.7E−01 | 3.96 | 5.5E−29 | −0.67 | 1.3E−01 |
| | PUL 11 | | | | | | | | |
| Bova172_1157 | Predicted | hypothetical protein | | 0.62 | 2.2E−01 | 4.09 | 2.0E−24 | −0.57 | 2.1E−01 |
| | PUL 11 | | | | | | | | |
| Bova172_1158 | Predicted | leucine-rich repeat protein, function | | 0.17 | 6.7E−01 | 3.64 | 3.4E−31 | −0.34 | 3.7E−01 |
| | PUL 11 | unknown | | | | | | | |
| Bova172_1159 | Predicted | hypothetical protein | | −0.11 | 8.3E−01 | 3.67 | 3.8E−22 | −1.07 | 3.0E−02 |
| | PUL 11 | | | | | | | | |
| Bova172_1160 | Predicted | hypothetical protein | | −0.20 | 7.4E−01 | 4.00 | 9.8E−10 | −0.06 | 9.2E−01 |
| | PUL 11 | | | | | | | | |
| Bova172_1161 | Predicted | Cell surface glycan-binding | SusD | −0.07 | 8.8E−01 | 3.47 | 1.2E−19 | 0.10 | 8.4E−01 |
| | PUL 11 | lipoprotein, SusD family | | | | | | | |
| Bova172_1162 | Predicted | Outer membrane TonB-dependent | Trans | −1.88 | 6.9E−10 | 4.09 | 3.2E−46 | −0.21 | 5.0E−01 |
| | PUL 11 | transporter, SusC family | | | | | | | |
| Bova172_1174 | Predicted | Uncharacterized MFS-type | Trans | −0.11 | 7.2E−01 | −0.33 | 1.6E−01 | 0.20 | 6.3E−01 |
| | PUL 12 | transporter | | | | | | | |
| Bova172_1175 | Predicted | Glycerophosphoryl diester | | −0.74 | 3.5E−02 | −0.94 | 1.3E−03 | 0.04 | 9.5E−01 |
| | PUL 12 | phosphodiesterase (EC 3.1.4.46) | | | | | | | |
| Bova172_1176 | Predicted | hypothetical protein | | −0.77 | 1.0E−02 | −0.80 | 2.7E−03 | −1.27 | 1.4E−02 |
| | PUL 12 | | | | | | | | |
| Bova172_1177 | Predicted | hypothetical protein | | −0.67 | 1.1E−01 | −0.69 | 3.5E−02 | −0.30 | 5.4E−01 |
| | PUL 12 | | | | | | | | |
| Bova172_1178 | Predicted | Cell surface protein | | −0.91 | 4.2E−04 | −1.16 | 1.8E−09 | −0.47 | 2.4E−01 |
| | PUL 12 | | | | | | | | |
| Bova172_1179 | Predicted | Cell surface glycan-binding | SusD | −0.48 | 1.6E−01 | −1.31 | 6.8E−08 | −1.27 | 1.5E−02 |
| | PUL 12 | lipoprotein, SusD family | | | | | | | |
| Bova172_1180 | Predicted | Outer membrane TonB-dependent | Trans | −1.06 | 5.1E−14 | −1.80 | 5.1E−49 | −1.18 | 2.4E−06 |
| | PUL 12 | transporter, SusC family | | | | | | | |
| Bova172_1181 | Predicted | hypothetical protein | | −0.32 | 4.6E−01 | −2.35 | 4.0E−10 | −0.67 | 2.2E−01 |
| | PUL 12 | | | | | | | | |
| Bova172_1182 | Predicted | Putative anti-sigma factor | REG | 0.51 | 2.1E−04 | −2.74 | 3.7E−94 | −0.21 | 3.7E−01 |
| | PUL 12 | | | | | | | | |
| Bova172_1183 | Predicted | RNA polymerase ECF-type sigma | REG | −0.78 | 3.4E−06 | −2.25 | 2.4E−43 | −0.05 | 9.1E−01 |
| | PUL 12 | factor | | | | | | | |
| Bova172_1221 | Predicted | hypothetical protein | | 1.57 | 1.6E−31 | −2.01 | 6.8E−60 | 2.09 | 2.0E−33 |
| | PUL 13 | | | | | | | | |
| Bova172_1222 | Predicted | unsaturated glucuronyl hydrolase | GH88 | 0.11 | 7.2E−01 | −0.76 | 7.1E−03 | −0.12 | 8.3E−01 |
| | PUL 13 | | | | | | | | |
| Bova172_1223 | Predicted | α-L/B-D-glycosidase | GH43_10 | −0.10 | 6.6E−01 | −1.19 | 2.3E−08 | 0.15 | 7.1E−01 |
| | PUL 13 | | | | | | | | |
| Bova172_{—}1224 | Predicted | alpha-galactosidase (EC 3.2.1.22) | GH36 | 1.96 | 1.3E−10 | 0.13 | 6.1E−01 | −0.09 | 8.7E−01 |
| | PUL 13 | | | | | | | | |
| Bova172_1225 | Predicted | hypothetical protein | | 1.56 | 1.3E−12 | −0.02 | 9.3E−01 | −0.36 | 4.1E−01 |
| | PUL 13 | | | | | | | | |
| Bova172_1226 | Predicted | hypothetical protein | | 1.53 | 1.4E−11 | −0.17 | 4.6E−01 | 0.15 | 7.5E−01 |
| | PUL 13 | | | | | | | | |
| Bova172_{—}1227 | Predicted | Cell surface glycan-binding | SusD | 1.80 | 2.4E−15 | −0.26 | 2.2E−01 | 0.35 | 3.3E−01 |
| | PUL 13 | lipoprotein, SusD family | | | | | | | |
| Bova172_{—}1228 | Predicted | Outer membrane TonB-dependent | Trans | 1.53 | 1.6E−29 | −0.38 | 7.4E−03 | 0.14 | 6.0E−01 |
| | PUL 13 | transporter, SusC family | | | | | | | |
| Bova172_1229 | Predicted | hypothetical protein | | −0.06 | 9.2E−01 | −0.90 | 1.6E−01 | 0.14 | 1.0E+00 |
| | PUL 13 | | | | | | | | |
| Bova172_{—}1230 | Predicted | RNA polymerase ECF-type sigma | REG | 1.77 | 2.0E−04 | −1.11 | 1.8E−02 | −0.07 | 8.9E−01 |
| | PUL 13 | factor | | | | | | | |
| Bova172_1231 | Predicted | DNA-binding response regulator, | REG | −0.29 | 4.1E−03 | −0.46 | 2.6E−07 | −0.32 | 1.5E−01 |
| | PUL 13 | AraC family | | | | | | | |
| Bova172_1234 | Predicted | beta-galactosidase (EC 3.2.1.23) | GH2 | 0.08 | 8.4E−01 | 0.35 | 1.8E−01 | 0.66 | 1.1E−01 |
| | PUL 14 | | | | | | | | |
| Bova172_1235 | Predicted | hypothetical protein | | 0.25 | 5.0E−01 | −0.27 | 3.9E−01 | 0.82 | 1.3E−01 |
| | PUL 14 | | | | | | | | |
| Bova172_1236 | Predicted | hypothetical protein | | −0.98 | 1.7E−02 | 0.87 | 4.5E−02 | 0.24 | 1.0E+00 |
| | PUL 14 | | | | | | | | |
| Bova172_1237 | Predicted | Cell surface glycan-binding | SusD | −1.20 | 1.4E−04 | 0.05 | 8.8E−01 | 0.04 | 9.5E−01 |
| | PUL 14 | lipoprotein, SusD family | | | | | | | |
| Bova172_1238 | Predicted | Outer membrane TonB-dependent | Trans | −0.62 | 6.5E−03 | −0.64 | 4.6E−03 | −0.64 | 1.2E−01 |
| | PUL 14 | transporter, SusC family | | | | | | | |
| Bova172_1239 | Predicted | RNA polymerase ECF-type sigma | REG | −0.57 | 2.6E−01 | 0.21 | 6.8E−01 | 0.41 | 1.0E+00 |
| | PUL 14 | factor | | | | | | | |
| Bova172_1240 | Predicted | hypothetical protein | | −0.58 | 3.2E−01 | −0.25 | 6.5E−01 | −0.07 | 1.0E+00 |
| | PUL 14 | | | | | | | | |
| Bova172_1241 | Predicted | DNA-binding response regulator, | REG | −1.42 | 1.1E−18 | 0.58 | 7.1E−05 | −0.39 | 2.1E−01 |
| | PUL 14 | AraC family | | | | | | | |
| Bova172_1242 | Predicted | hypothetical protein | PL | 0.80 | 4.3E−04 | 1.57 | 3.3E−17 | 0.11 | 7.7E−01 |
| | PUL 14 | | | | | | | | |
| Bova172_1243 | Predicted | Beta-lactamase class C-like and | Pept | 1.07 | 1.3E−04 | 1.76 | 4.6E−12 | −0.13 | 7.8E−01 |
| | PUL 14 | penicillin binding proteins (PBPs) | | | | | | | |
| | | superfamily | | | | | | | |
| Bova172_1244 | Predicted | Putative glycosyl hydrolase of | GH146 | 0.65 | 3.4E−03 | 1.07 | 8.9E−09 | 0.06 | 8.9E−01 |
| | PUL 14 | unknown function (DUF1680) | | | | | | | |
| Bova172_1261 | Predicted | hypothetical protein | GH28 | 0.12 | 8.2E−01 | −0.10 | 8.0E−01 | −0.15 | 1.0E+00 |
| | PUL 15 | | | | | | | | |
| Bova172_1262 | Predicted | hypothetical protein | | −0.42 | 3.6E−01 | −1.07 | 4.5E−03 | 0.37 | 4.7E−01 |
| | PUL 15 | | | | | | | | |
| Bova172_1263 | Predicted | hypothetical protein | | 1.61 | 2.5E−03 | −0.78 | 2.3E−02 | 0.11 | 8.4E−01 |
| | PUL 15 | | | | | | | | |
| Bova172_1264 | Predicted | hypothetical protein | | 0.14 | 7.8E−01 | −0.22 | 5.8E−01 | −0.15 | 1.0E+00 |
| | PUL 15 | | | | | | | | |
| Bova172_1265 | Predicted | Cell surface glycan-binding | SusD | 0.27 | 5.0E−01 | −0.68 | 1.9E−02 | 0.91 | 6.7E−02 |
| | PUL 15 | lipoprotein, SusD family | | | | | | | |
| Bova172_1266 | Predicted | Outer membrane TonB-dependent | Trans | −0.02 | 9.6E−01 | −0.36 | 2.0E−01 | 0.20 | 7.0E−01 |
| | PUL 15 | transporter, SusC family | | | | | | | |
| Bova172_1267 | Predicted | RNA polymerase ECF-type sigma | REG | −0.52 | 2.8E−01 | −0.40 | 4.4E−01 | 0.14 | 1.0E+00 |
| | PUL 15 | factor | | | | | | | |
| Bova172_1268 | Predicted | hypothetical protein | | 2.75 | 1.1E−121 | −0.45 | 2.7E−05 | 3.13 | 9.0E−103 |
| | PUL 15 | | | | | | | | |
| Bova172_1269 | Predicted | BaeS-type histidine kinase/ | REG | −0.94 | 2.5E−10 | −0.39 | 4.5E−03 | −0.55 | 9.7E−02 |
| | PUL 15 | OmpR-type DNA-binding response | | | | | | | |
| | | regulator | | | | | | | |
| Bova172_1270 | Predicted | COG5434 Endopygalactorunase | CBM6 | −0.76 | 1.3E−12 | −1.48 | 2.4E−45 | 0.23 | 2.1E−01 |
| | PUL 15 | | | | | | | | |
| Bova172_{—}1271 | Predicted | COG5434 Endopygalactorunase | GH28 | 1.19 | 1.0E−02 | −0.44 | 3.5E−01 | 0.03 | 9.5E−01 |
| | PUL 15 | | | | | | | | |
| Bova172_1306 | Predicted | hypothetical protein | | 0.10 | 7.8E−01 | −0.11 | 7.0E−01 | −0.20 | 6.8E−01 |
| | PUL 16 | | | | | | | | |
| Bova172_1307 | Predicted | related to competence proteins | Sulf | 0.36 | 1.7E−01 | −0.54 | 1.4E−02 | 0.13 | 7.7E−01 |
| | PUL 16 | | | | | | | | |
| Bova172_1308 | Predicted | hypothetical protein | | 0.41 | 1.7E−01 | −1.07 | 6.1E−05 | 0.58 | 1.4E−01 |
| | PUL 16 | | | | | | | | |
| Bova172_1309 | Predicted | hypothetical protein | | 0.40 | 4.2E−01 | −0.01 | 9.9E−01 | 0.12 | 8.3E−01 |
| | PUL 16 | | | | | | | | |
| Bova172_1310 | Predicted | Cell surface glycan-binding | SusD | 0.70 | 1.1E−01 | −0.21 | 5.3E−01 | 0.57 | 2.7E−01 |
| | PUL 16 | lipoprotein, SusD family | | | | | | | |
| Bova172_1311 | Predicted | Outer membrane TonB-dependent | Trans | 1.53 | 1.7E−09 | −0.90 | 9.1E−07 | 0.85 | 2.5E−02 |
| | PUL 16 | transporter, SusC family | | | | | | | |
| Bova172_1312 | Predicted | hypothetical protein | | 0.48 | 1.8E−13 | −1.87 | 9.3E−186 | 0.39 | 9.3E−08 |
| | PUL 16 | | | | | | | | |
| Bova172_{—}1313 | Predicted | Iron siderophore sensor protein | REG | 1.24 | 3.3E−15 | −1.60 | 8.0E−31 | −0.08 | 8.3E−01 |
| | PUL 16 | | | | | | | | |
| Bova172_1314 | Predicted | RNA polymerase ECF-type sigma | REG | 0.94 | 5.2E−13 | −2.83 | 2.7E−103 | −0.05 | 9.0E−01 |
| | PUL 16 | factor | | | | | | | |
| Bova172_1321 | Predicted | beta-glucosidase (EC 3.2.1.21) | GH3 | 0.99 | 2.5E−03 | −0.15 | 5.2E−01 | 0.89 | 7.6E−02 |
| | PUL 17 | | | | | | | | |
| Bova172_1322 | Predicted | hypothetical protein | | −0.12 | 7.9E−01 | −0.38 | 2.2E−01 | 0.04 | 9.5E−01 |
| | PUL 17 | | | | | | | | |
| Bova172_1323 | Predicted | hypothetical protein | | −0.86 | 2.3E−03 | −0.72 | 5.4E−04 | −0.25 | 6.0E−01 |
| | PUL 17 | | | | | | | | |
| Bova172_1324 | Predicted | hypothetical protein | | −0.63 | 3.7E−02 | −1.45 | 3.0E−10 | 0.05 | 9.4E−01 |
| | PUL 17 | | | | | | | | |
| Bova172_1325 | Predicted | Endo-beta-N- | GH18 | −0.83 | 7.9E−03 | −1.08 | 6.4E−06 | −0.57 | 1.8E−01 |
| | PUL 17 | acetylglucosaminidase F2 | | | | | | | |
| Bova172_1326 | Predicted | Cell surface glycan-binding | SusD | −0.15 | 5.9E−01 | −1.40 | 6.3E−12 | −0.14 | 7.7E−01 |
| | PUL 17 | lipoprotein, SusD family | | | | | | | |
| Bova172_1327 | Predicted | Outer membrane TonB-dependent | Trans | 0.24 | 9.5E−02 | −2.33 | 4.9E−88 | −0.23 | 3.7E−01 |
| | PUL 17 | transporter, SusC family | | | | | | | |
| Bova172_1328 | Predicted | Putative anti-sigma factor | REG | −0.47 | 1.8E−09 | −3.81 | 0.0E+00 | 0.44 | 2.8E−03 |
| | PUL 17 | | | | | | | | |
| Bova172_1329 | Predicted | Serine/threonine kinase | GH33 | −0.46 | 6.8E−05 | −2.01 | 7.8E−60 | 0.14 | 6.6E−01 |
| | PUL 17 | | | | | | | | |
| Bova172_1330 | Predicted | RNA polymerase ECF-type sigma | REG | −0.46 | 1.1E−03 | −1.60 | 4.4E−33 | 0.60 | 1.5E−02 |
| | PUL 17 | factor | | | | | | | |
| Bova172_1339 | Predicted | Acetyltransferase, GNAT family | | −0.53 | 2.2E−02 | 1.99 | 2.7E−26 | −0.04 | 9.5E−01 |
| | PUL 18 | | | | | | | | |
| Bova172_1340 | Predicted | 23S rRNA (adenine(1618)-N(6))- | | −1.04 | 3.0E−11 | 1.27 | 2.1E−21 | −0.55 | 2.2E−01 |
| | PUL 18 | methyltransferase (EC 2.1.1.181) | | | | | | | |
| Bova172_1341 | Predicted | Two-component transcriptional | | −0.03 | 9.7E−01 | 1.43 | 1.2E−02 | 0.03 | 1.0E+00 |
| | PUL 18 | response regulator, LuxR family | | | | | | | |
| Bova172_1342 | Predicted | Cell surface glycan-binding | SusD | 0.20 | 7.0E−01 | 2.02 | 2.9E−06 | −0.14 | 7.9E−01 |
| | PUL 18 | lipoprotein, SusD family | | | | | | | |
| Bova172_1343 | Predicted | Outer membrane TonB-dependent | Trans | 1.17 | 1.5E−04 | 1.21 | 5.3E−09 | 0.32 | 4.8E−01 |
| | PUL 18 | transporter, SusC family | | | | | | | |
| Tova172_1369 | Predicted | heparin lyase I precursor | PL13 | −0.01 | 9.4E−01 | −1.78 | 6.1E−31 | 0.20 | 5.8E−01 |
| | PUL 19 | (EC: 4.2.2.7) | | | | | | | |
| Bova172_1370 | Predicted | hypothetical protein | | 1.02 | 2.2E−34 | −2.26 | 4.7E−168 | 1.98 | 3.1E−72 |
| | PUL 19 | | | | | | | | |
| Bova172_1371 | Predicted | hypothetical protein | REG | −1.28 | 4.3E−49 | 0.44 | 1.3E−07 | 0.17 | 2.0E−01 |
| | PUL 19 | | | | | | | | |
| Bova172_1372 | Predicted | hypothetical protein | GH147 | 3.30 | 1.0E+00 | 2.19 | 1.0E+00 | 1.32 | 1.0E+00 |
| | PUL 19 | | | | | | | | |
| Bova172_1373 | Predicted | Outer membrane TonB-dependent | Trans | 2.92 | 7.1E−23 | 1.47 | 1.9E−06 | 2.82 | 1.3E−15 |
| | PUL 19 | transporter, SusC family | | | | | | | |
| Bova172_1374 | Predicted | Cell surface glycan-binding | SusD | 4.02 | 2.1E−28 | 1.20 | 1.2E−03 | 3.24 | 3.6E−11 |
| | PUL 19 | lipoprotein, SusD family | | | | | | | |
| Bova172_1375 | Predicted | hypothetical protein | | 4.16 | 9.1E−20 | 0.72 | 9.1E−02 | 2.42 | 3.0E−06 |
| | PUL 19 | | | | | | | | |
| Bova172_1376 | Predicted | arabinogalactan endo-1,4-beta- | GH53 | 4.07 | 3.8E−23 | 0.61 | 1.4E−01 | 2.67 | 5.9E−06 |
| | PUL 19 | galactosidase | | | | | | | |
| Bova172_1377 | Predicted | beta-galactosidase (EC 3.2.1.23) | GH2 | 0.07 | 5.6E−01 | −0.04 | 7.2E−01 | −0.25 | 1.3E−01 |
| | PUL 19 | | | | | | | | |
| Bova172_1388 | Predicted | BaeS-type histidine kinase/ | REG | −1.30 | 6.4E−46 | 0.56 | 5.2E−12 | −1.34 | 4.5E−09 |
| | PUL 20 | OmpR-type DNA-binding response | | | | | | | |
| | | regulator | | | | | | | |
| Bova172_1389 | Predicted | Uncharacterized protein RB4699 | GH95 | −1.01 | 1.7E−02 | 0.53 | 1.5E−01 | 0.24 | 6.2E−01 |
| | PUL 20 | | | | | | | | |
| Bova172_1390 | Predicted | hypothetical protein | PL12_2 | −2.47 | 6.5E−45 | −2.09 | 5.3E−45 | −1.64 | 6.0E−05 |
| | PUL 20 | | | | | | | | |
| Bova172_1391 | Predicted | hypothetical protein | | −1.67 | 1.8E−12 | −1.15 | 1.8E−09 | −1.32 | 6.1E−03 |
| | PUL 20 | | | | | | | | |
| Bova172_1392 | Predicted | Outer membrane TonB-dependent | Trans | −1.32 | 1.6E−08 | −0.89 | 7.9E−06 | −0.83 | 2.1E−02 |
| | PUL 20 | transporter, SusC family | | | | | | | |
| Bova172_1393 | Predicted | Cell surface glycan-binding | SusD | −0.43 | 1.9E−01 | −0.59 | 1.4E−02 | −1.09 | 3.3E−02 |
| | PUL 20 | lipoprotein, SusD family | | | | | | | |
| Bova172_1394 | Predicted | Glucuronyl hydrolase | GH88 | 0.07 | 9.0E−01 | −0.07 | 8.7E−01 | 0.43 | 3.9E−01 |
| | PUL 20 | | | | | | | | |
| Bova172_1395 | Predicted | heparinase III protein | PL12_2 | −1.33 | 2.6E−05 | 0.39 | 1.9E−01 | −0.41 | 4.0E−01 |
| | PUL 20 | | | | | | | | |
| Bova172_1396 | Predicted | Mucin-desulfating sulfatase | Sulf | −0.45 | 3.4E−01 | 0.37 | 3.8E−01 | 0.23 | 1.0E+00 |
| | PUL 20 | | | | | | | | |
| Bova172_1397 | Predicted | hypothetical protein | | −0.45 | 5.6E−02 | −1.13 | 8.8E−08 | 0.47 | 2.4E−01 |
| | PUL 20 | | | | | | | | |
| Bova172_1398 | Predicted | ROK family protein (putative | ROK | 0.80 | 1.1E−02 | −1.39 | 6.1E−10 | 0.33 | 4.7E−01 |
| | PUL 20 | glucokinase) | | | | | | | |
| Bova172_1399 | Predicted | Hypothetical sugar permease | Trans | 0.68 | 1.8E−01 | −0.14 | 7.0E−01 | 0.27 | 5.9E−01 |
| | PUL 20 | | | | | | | | |
| Bova172_1400 | Predicted | hypothetical protein | PL15_2 | 1.27 | 1.2E−04 | −0.28 | 2.3E−01 | −0.06 | 9.1E−01 |
| | PUL 20 | | | | | | | | |
| Bova172_1526 | Predicted | hypothetical protein | | 0.69 | 4.3E−02 | 2.70 | 1.3E−18 | 0.25 | 6.1E−01 |
| | PUL 21 | | | | | | | | |
| Bova172_1527 | Predicted | Cell surface glycan-binding | SusD | 0.03 | 8.7E−01 | 1.69 | 8.0E−19 | −0.52 | 1.4E−01 |
| | PUL 21 | lipoprotein, SusD family | | | | | | | |
| Bova172_1528 | Predicted | Outer membrane TonB-dependent | Trans | −0.59 | 1.9E−08 | 0.22 | 2.8E−02 | −0.44 | 3.3E−02 |
| | PUL 21 | transporter, SusC family | | | | | | | |
| Bova172_1546 | Predicted | Fructokinase (EC 2.7.1.4) | | 0.15 | 7.8E−01 | 0.10 | 8.2E−01 | 0.04 | 1.0E+00 |
| | PUL 22 | | | | | | | | |
| Bova172_1547 | Predicted | Alcohol dehydrogenase (EC | | −2.00 | 4.9E−04 | 1.20 | 4.2E−02 | 0.12 | 1.0E+00 |
| | PUL 22 | 1.1.1.1) | | | | | | | |
| Bova172_1548 | Predicted | Platelet-activating factor | | −0.37 | 5.0E−01 | 1.34 | 2.6E−02 | −0.09 | 1.0E+00 |
| | PUL 22 | acetylhydrolase IB gamma subunit | | | | | | | |
| | | (EC 3.1.1.47) | | | | | | | |
| Bova172_1549 | Predicted | Multiple polyol-specific | | −0.58 | 2.7E−01 | 0.35 | 4.2E−01 | 0.23 | 1.0E+00 |
| | PUL 22 | dehydrogenase (EC 1.1.1.—) | | | | | | | |
| Bova172_1550 | Predicted | hypothetical protein | | −0.89 | 3.8E−02 | 1.11 | 1.4E−02 | 0.28 | 5.7E−01 |
| | PUL 22 | | | | | | | | |
| Bova172_1551 | Predicted | hypothetical protein | | −1.82 | 4.4E−05 | 0.48 | 2.6E−01 | −0.36 | 1.0E+00 |
| | PUL 22 | | | | | | | | |
| Bova172_1552 | Predicted | hypothetical protein | | −0.68 | 1.1E−01 | 0.19 | 6.5E−01 | −0.19 | 7.1E−01 |
| | PUL 22 | | | | | | | | |
| Bova172_1553 | Predicted | hypothetical protein | | −0.97 | 9.9E−02 | −0.20 | 7.2E−01 | 0.25 | 1.0E+00 |
| | PUL 22 | | | | | | | | |
| Bova172_1554 | Predicted | hypothetical protein | | −2.03 | 7.4E−05 | −0.21 | 6.2E−01 | 0.03 | 1.0E+00 |
| | PUL 22 | | | | | | | | |
| Bova172_1555 | Predicted | Cell surface glycan-binding | SusD | −0.98 | 3.1E−03 | 0.00 | 1.0E−00 | 0.63 | 1.6E−01 |
| | PUL 22 | lipoprotein, SusD family | | | | | | | |
| Bova172_1556 | Predicted | Outer membrane TonB-dependent | Trans | −0.55 | 6.6E−02 | 0.43 | 1.9E−01 | 0.29 | 5.1E−01 |
| | PUL 22 | transporter, SusC family | | | | | | | |
| Bova172_1557 | Predicted | hypothetical protein | | −0.22 | 5.5E−01 | −0.49 | 1.4E−01 | −0.39 | 3.7E−01 |
| | PUL 22 | | | | | | | | |
| Bova172_1558 | Predicted | hypothetical protein | | −0.85 | 7.5E−04 | −0.55 | 4.7E−02 | 0.42 | 3.0E−01 |
| | PUL 22 | | | | | | | | |
| Bova172_1559 | Predicted | FAD dependent oxidoreductase | | −0.21 | 3.7E−01 | −1.82 | 3.1E−10 | 0.49 | 1.4E−01 |
| | PUL 22 | | | | | | | | |
| Bova172_1660 | Predicted | Glycerophosphoryl diester | | 0.32 | 5.6E−01 | 0.74 | 1.6E−01 | −0.39 | 1.0E+00 |
| | PUL 23 | phosphodiesterase (EC 3.1.4.46) | | | | | | | |
| Bova172_1661 | Predicted | Cell surface glycan-binding | SusD | 0.05 | 9.1E−01 | 1.50 | 6.7E−05 | −0.54 | 2.5E−01 |
| | PUL 23 | lipoprotein, SusD family | | | | | | | |
| Bova172_1662 | Predicted | Outer membrane TonB-dependent | Trans | 0.25 | 5.4E−01 | 1.69 | 2.0E−06 | −1.02 | 1.1E−02 |
| | PUL 23 | transporter, SusC family | | | | | | | |
| Bova172_1663 | Predicted | hypothetical protein | | −0.84 | 1.7E−01 | −0.16 | 7.8E−01 | 0.14 | 1.0E+00 |
| | PUL 23 | | | | | | | | |
| Bova172_{—}1664 | Predicted | BaeS-type histidine kinase/ | REG | 2.21 | 1.3E−19 | −0.32 | 4.4E−02 | −0.31 | 2.2E−01 |
| | PUL 23 | OmpR-type DNA-binding response | | | | | | | |
| | | regulator | | | | | | | |
| Bova172_1787 | Predicted | hypothetical protein | | −0.34 | 1.9E−01 | 0.07 | 8.0E−01 | 0.38 | 3.0E−01 |
| | PUL 24 | | | | | | | | |
| Bova172_1788 | Predicted | hypothetical protein | | −0.42 | 2.6E−01 | 0.20 | 6.0E−01 | −0.29 | 5.5E−01 |
| | PUL 24 | | | | | | | | |
| Bova172_1789 | Predicted | Cell surface glycan-binding | SusD | −1.77 | 1.4E−07 | −0.28 | 4.1E−01 | −1.40 | 1.8E−02 |
| | PUL 24 | lipoprotein, SusD family | | | | | | | |
| Bova172_1790 | Predicted | Outer membrane TonB-dependent | Trans | −1.46 | 1.2E−21 | −1.41 | 5.6E−18 | −1.59 | 3.3E−08 |
| | PUL 24 | transporter, SusC family | | | | | | | |
| Bova172_1791 | Predicted | Putative anti-sigma factor | REG | −1.64 | 2.1E−20 | −1.94 | 9.6E−21 | −0.55 | 8.8E−02 |
| | PUL 24 | | | | | | | | |
| Bova172_1792 | Predicted | RNA polymerase ECF-type sigma | REG | −0.42 | 1.5E−02 | −2.45 | 7.3E−49 | 0.32 | 2.4E−01 |
| | PUL 24 | factor | | | | | | | |
| Bova172_1793 | Predicted | Alpha-N-acetylglucosaminidase | GH89 | −0.48 | 2.2E−04 | −0.68 | 1.2E−10 | −0.69 | 8.3E−03 |
| | PUL 25 | (EC 3.2.1.50) | | | | | | | |
| Bova172_1794 | Predicted | beta-galactosidase (EC 3.2.1.23) | CBM32- | −0.56 | 7.6E−02 | 1.35 | 7.6E−06 | −0.55 | 2.6E−01 |
| | PUL 25 | | GH35 | | | | | | |
| Bova172_1795 | Predicted | hypothetical protein | | −0.24 | 3.8E−01 | −0.90 | 7.0E−06 | −0.63 | 1.3E−01 |
| | PUL 25 | | | | | | | | |
| Bova172_1796 | Predicted | hypothetical protein | | −1.36 | 3.3E−05 | −0.68 | 1.1E−02 | −0.14 | 7.7E−01 |
| | PUL 25 | | | | | | | | |
| Bova172_1797 | Predicted | Cell surface glycan-binding | SusD | −1.39 | 4.6E−05 | −0.88 | 1.0E−03 | −0.59 | 2.1E−01 |
| | PUL 25 | lipoprotein, SusD family | | | | | | | |
| Bova172_1798 | Predicted | Outer membrane TonB-dependent | Trans | −0.44 | 1.8E−02 | −1.53 | 1.2E−29 | −0.94 | 1.8E−03 |
| | PUL 25 | transporter, SusC family | | | | | | | |
| Bova172_1799 | Predicted | Putative anti-sigma factor | REG | 0.81 | 1.4E−11 | −2.81 | 6.2E−137 | −0.17 | 3.5E−01 |
| | PUL 25 | | | | | | | | |
| Bova172_1800 | Predicted | FIG00416925: hypothetical protein | | — | — | — | — | — | — |
| | PUL 25 | | | | | | | | |
| Bova172_1801 | Predicted | RNA polymerase ECF-type sigma | REG | 0.20 | 2.4E−01 | −1.84 | 1.2E−31 | 2.04 | 3.7E−19 |
| | PUL 25 | factor | | | | | | | |
| Bova172_1824 | Predicted | RNA polymerase sigma-70 factor | REG | −0.41 | 3.3E−03 | 1.20 | 1.7E−17 | 0.91 | 1.1E−03 |
| | PUL 26 | | | | | | | | |
| Bova172_1825 | Predicted | Putative anti-sigma factor | REG | 0.70 | 1.0E−02 | 0.06 | 7.9E−01 | 0.72 | 7.2E−02 |
| | PUL 26 | | | | | | | | |
| Bova172_1826 | Predicted | Outer membrane TonB-dependent | Trans | 1.65 | 1.9E−05 | 1.50 | 1.1E−09 | 0.52 | 2.1E−01 |
| | PUL 26 | transporter, SusC family | | | | | | | |
| Bova172_1827 | Predicted | Cell surface glycan-binding | SusD | 0.88 | 9.4E−02 | 1.51 | 6.9E−05 | 0.33 | 5.0E−01 |
| | PUL 26 | lipoprotein, SusD family | | | | | | | |
| Bova172_1828 | Predicted | hypothetical protein | | 0.27 | 6.4E−01 | 1.38 | 2.8E−03 | 0.24 | 1.0E+00 |
| | PUL 26 | | | | | | | | |
| Bova172_1829 | Predicted | Thioredoxin family protein | | 0.29 | 6.1E−01 | 1.72 | 1.0E−03 | 0.13 | 1.0E+00 |
| | PUL 26 | | | | | | | | |
| Bova172_1830 | Predicted | hypothetical protein | Pept | 0.09 | 8.6E−01 | 1.29 | 5.9E−04 | 0.04 | 9.5E−01 |
| | PUL 26 | | | | | | | | |
| Bova172_1831 | Predicted | hypothetical protein | | −0.07 | 9.0E−01 | 2.45 | 4.5E−06 | 0.13 | 1.0E+00 |
| | PUL 26 | | | | | | | | |
| Bova172_1832 | Predicted | hypothetical protein | | 0.10 | 8.7E−01 | 2.29 | 2.0E−05 | 0.44 | 1.0E+00 |
| | PUL 26 | | | | | | | | |
| Bova172_1833 | Predicted | Thioredoxin | | 0.47 | 4.0E−01 | 2.56 | 3.7E−07 | 0.05 | 9.5E−01 |
| | PUL 26 | | | | | | | | |
| Bova172_1834 | Predicted | beta-glycosyl hydrolase | GH20 | −1.22 | 6.5E−47 | −1.07 | 2.6E−45 | −0.40 | 1.0E−02 |
| | PUL 26 | | | | | | | | |
| Bova172_1884 | CAZyme | alpha-N-acetylgalactosaminidase | GH109 | 0.69 | 8.8E−03 | 2.09 | 1.9E−19 | −0.34 | 4.3E−01 |
| | cluster 3 | (EC 3.2.1.49) | | | | | | | |
| Bova172_1885 | CAZyme | Oxidoreductase, Gfo/Idh/MocA | GH109 | −0.82 | 1.5E−27 | −0.90 | 9.6E−38 | −0.30 | 1.6E−02 |
| | cluster 3 | family | | | | | | | |
| Bova172_1886 | CAZyme | Uncharacterized membrane- | | −1.28 | 4.8E−26 | −0.35 | 2.3E−03 | −1.51 | 7.9E−06 |
| | cluster 3 | anchored protein, YitT family | | | | | | | |
| Bova172_1887 | CAZyme | beta-galactosidase (EC 3.2.1.23) | GH2 | −0.83 | 4.0E−18 | 0.35 | 1.2E−04 | −1.62 | 1.0E−13 |
| | cluster 3 | | | | | | | | |
| Bova172_1888 | CAZyme | MdsC protein | | 0.23 | 6.3E−02 | 1.75 | 6.9E−78 | −1.62 | 2.7E−08 |
| | cluster 3 | | | | | | | | |
| Bova172_1932 | Predicted | unsaturated glucuronyl hydrolase | GH105 | 2.73 | 1.1E−19 | 0.04 | 9.0E−01 | 1.19 | 9.6E−03 |
| | PUL 27 | | | | | | | | |
| Bova172_1933 | Predicted | BaeS-type histidine kinase/ | REG | 0.99 | 4.0E−20 | −0.65 | 7.0E−13 | 0.12 | 6.4E−01 |
| | PUL 27 | OmpR-type DNA-binding response | HTCS_Rgu- | | | | | | |
| | | regulator | 2 | | | | | | |
| Bova172_1934 | Predicted | hypothetical protein | | −0.11 | 8.5E−01 | −1.15 | 7.1E−02 | 0.02 | 1.0E+00 |
| | PUL 27 | | | | | | | | |
| Bova172_1935 | Predicted | possible β-polygalacturonase | GH28 | 2.00 | 5.4E−05 | 0.66 | 4.5E−02 | −0.32 | 5.4E−01 |
| | PUL 27 | | | | | | | | |
| Bova172_1936 | Predicted | hypothetical protein | GH | 1.01 | 1.0E−03 | 0.13 | 6.4E−01 | 0.99 | 1.2E−02 |
| | PUL 27 | | | | | | | | |
| Bova172_1937 | Predicted | Polygalacturonase (EC 3.2.1.15) | | 0.68 | 5.0E−03 | −1.05 | 2.0E−06 | 0.87 | 3.9E−03 |
| | PUL 27 | | | | | | | | |
| Bova172_1938 | Predicted | hypothetical protein | SusD | 4.56 | 4.0E−18 | 0.56 | 1.3E−01 | 0.40 | 4.3E−01 |
| | PUL 27 | | | | | | | | |
| Bova172_{—}1939 | Predicted | Outer membrane TonB-dependent | Trans | 4.26 | 4.5E−23 | 0.10 | 7.6E−01 | 0.67 | 1.6E−01 |
| | PUL 27 | transporter, SusC family | | | | | | | |
| Bova172_1940 | Predicted | hypothetical protein | | 3.68 | 1.7E−17 | −0.18 | 6.2E−01 | 0.83 | 1.3E−01 |
| | PUL 27 | | | | | | | | |
| Bova172_1941 | Predicted | hypothetical protein | | 4.88 | 1.7E−18 | 0.52 | 2.2E−01 | 0.36 | 4.5E−01 |
| | PUL 27 | | | | | | | | |
| Bova172_{—}1942 | Predicted | Cell surface glycan-binding | SusD | 4.79 | 7.9E−27 | 0.45 | 2.9E−01 | 0.58 | 2.5E−01 |
| | PUL 27 | lipoprotein, SusD family | | | | | | | |
| Bova172_1943 | Predicted | Outer membrane TonB-dependent | Trans | 5.71 | 1.6E−65 | 0.00 | 1.0E+00 | 1.36 | 1.1E−03 |
| | PUL 27 | transporter, SusC family | | | | | | | |
| Bova172_1944 | Predicted | hypothetical protein | | 5.13 | 5.0E−69 | −0.60 | 3.7E−02 | 1.23 | 3.4E−04 |
| | PUL 27 | | | | | | | | |
| Bova172_1945 | Predicted | hypothetical protein | | 4.92 | 3.4E−18 | −0.21 | 6.8E−01 | 0.02 | 9.6E−01 |
| | PUL 27 | | | | | | | | |
| Bova172_1946 | Predicted | polysaccharide lyase | PL1_2 | 5.70 | 9.3E−57 | −0.32 | 3.5E−01 | 1.11 | 2.4E−02 |
| | PUL 27 | | | | | | | | |
| Bova172_1947 | Predicted | hypothetical protein | | 6.95 | 2.9E−31 | −0.64 | 5.4E−02 | 0.51 | 3.0E−01 |
| | PUL 27 | | | | | | | | |
| Bova172_{—}1948 | Predicted | Unsaturated glucuronyl hydrolase | GH105 | 6.08 | 1.1E−53 | 0.23 | 4.9E−01 | 0.63 | 1.9E−01 |
| | PUL 27 | | | | | | | | |
| Bova172_1949 | Predicted | α-galacturonidase | GH28 | 5.11 | 3.8E−20 | −0.11 | 8.0E−01 | 0.88 | 9.0E−02 |
| | PUL 27 | | | | | | | | |
| Bova172_1950 | Predicted | hypothetical protein | | — | — | — | — | — | — |
| | PUL 27 | | | | | | | | |
| Bova172_1951 | Predicted | hypothetical protein | | 2.52 | 9.2E−05 | −1.86 | 1.6E−04 | 0.13 | 1.0E+00 |
| | PUL 27 | | | | | | | | |
| Bova172_{—}1952 | Predicted | rhamnogalacturonan lyase | PL9 | 1.21 | 2.2E−04 | −2.14 | 1.6E−10 | 0.46 | 3.1E−01 |
| | PUL 27 | | | | | | | | |
| Bova172_{—}1953 | Predicted | α-galacturonidase | GH28 | 2.31 | 1.5E−06 | −0.35 | 4.7E−01 | 0.54 | 2.8E−01 |
| | PUL 27 | | | | | | | | |
| Bova172_{—}1954 | Predicted | rhamnogalacturonan lyase | PL11_1 | 1.59 | 3.0E−21 | −0.75 | 2.5E−04 | 0.16 | 5.9E−01 |
| | PUL 28 | | | | | | | | |
| Bova172_1955 | Predicted | BaeS-type histidine kinase/ | REG | −0.86 | 1.7E−16 | 0.20 | 3.8E−02 | −0.83 | 2.1E−05 |
| | PUL 28 | OmpR-type DNA-binding response | HTCS_Rgu- | | | | | | |
| | | regulator | 2 | | | | | | |
| Bova172_1956 | Predicted | β-glucuronidase | GH2 | 0.25 | 4.3E−01 | 1.99 | 1.4E−14 | −0.09 | 8.7E−01 |
| | PUL 28 | | | | | | | | |
| Bova172_1957 | Predicted | hypothetical protein | CE | −1.45 | 2.4E−02 | 0.20 | 7.1E−01 | 0.02 | 1.0E+00 |
| | PUL 28 | | | | | | | | |
| Bova172_1958 | Predicted | Outer membrane TonB-dependent | Trans | −1.25 | 2.8E−13 | −0.21 | 2.3E−01 | −0.23 | 5.0E−01 |
| | PUL 28 | transporter, SusC family | | | | | | | |
| Bova172_1959 | Predicted | Cell surface glycan-binding | SusD | −0.95 | 8.5E−05 | 1.10 | 5.0E−05 | 0.10 | 8.4E−01 |
| | PUL 28 | lipoprotein, SusD family | | | | | | | |
| Bova172_1960 | Predicted | hypothetical protein | | — | — | — | — | — | — |
| | PUL 28 | | | | | | | | |
| Bova172_1961 | Predicted | hypothetical protein | | 0.25 | 5.2E−02 | −2.69 | 3.5E−87 | 0.92 | 4.4E−09 |
| | PUL 28 | | | | | | | | |
| Bova172_1962 | Predicted | hypothetical protein | | −0.11 | 4.5E−01 | −2.41 | 2.4E−78 | 0.62 | 6.5E−04 |
| | PUL 28 | | | | | | | | |
| Bova172_1963 | Predicted | Pyridoxine 5′-phosphate synthase | | 1.12 | 1.3E−10 | −1.08 | 5.6E−16 | 2.02 | 1.4E−13 |
| | PUL 28 | (EC 2.6.99.2) | | | | | | | |
| Bova172_1964 | Predicted | rhamnogalacturonan | CE12 | 0.20 | 3.4E−01 | −0.25 | 1.4E−01 | 0.86 | 7.0E−03 |
| | PUL 28 | acetylesterase | | | | | | | |
| Bova172_1965 | Predicted | hypothetical protein | | −0.84 | 1.2E−03 | 0.39 | 1.2E−01 | 0.51 | 2.3E−01 |
| | PUL 28 | | | | | | | | |
| Bova172_1966 | Predicted | hypothetical protein | | −0.31 | 4.4E−01 | 0.38 | 3.1E−01 | 0.19 | 7.1E−01 |
| | PUL 28 | | | | | | | | |
| Bova172_1967 | Predicted | hypothetical protein | | 0.48 | 2.6E−01 | 0.24 | 5.0E−01 | −0.48 | 3.0E−01 |
| | PUL 28 | | | | | | | | |
| Bova172_1968 | Predicted | unsaturated rhamnogalacturonyl | GH105 | −0.61 | 1.6E−01 | 1.15 | 7.0E−03 | −0.46 | 3.6E−01 |
| | PUL 28 | hydrolase | | | | | | | |
| Bova172_1969 | Predicted | Polysaccharide deacetylase | CE | −0.52 | 2.4E−01 | 0.81 | 6.1E−02 | 0.44 | 1.0E+00 |
| | PUL 28 | | | | | | | | |
| Bova172_1970 | Predicted | BaeS-type histidine kinase/ | REG | −0.83 | 4.2E−15 | 1.09 | 1.2E−32 | −0.51 | 9.6E−02 |
| | PUL 28 | OmpR-type DNA-binding response | HTCS_Rgu- | | | | | | |
| | | regulator | 2 | | | | | | |
| Bova172_{—}1971 | Predicted | L-rhamnose mutarotase (EC | | 2.26 | 1.3E−03 | −1.86 | 2.2E−03 | 0.44 | 1.0E+00 |
| | PUL 28 | 5.1.3.32) | | | | | | | |
| Bova172_{—}1972 | Predicted | unsaturated rhamnogalacturonyl | GH105 | 2.43 | 1.5E−05 | 0.22 | 6.6E−01 | −0.25 | 6.3E−01 |
| | PUL 28 | hydrolase/RGI-disaccharide | | | | | | | |
| | | specific unsaturated | | | | | | | |
| | | galacturonidase | | | | | | | |
| Bova172_1973 | Predicted | rhamnogalacturonan lyase | PL11_1 | 2.96 | 1.6E−07 | −0.49 | 2.6E−01 | 1.27 | 5.1E−02 |
| | PUL 28 | | | | | | | | |
| Bova172_1974 | Predicted | unsaturated rhamnogalacturonyl | GH105 | 2.00 | 6.5E−07 | −0.03 | 9.4E−01 | 0.69 | 9.3E−02 |
| | PUL 28 | hydrolase/RGI-specific | | | | | | | |
| | | unsaturated galacturonidase | | | | | | | |
| Bova172_1975 | Predicted | rhamnogalacturonan | CE12-CE12 | 1.71 | 6.0E−06 | 1.42 | 1.4E−03 | −0.21 | 6.8E−01 |
| | PUL 28 | acetylesterase | | | | | | | |
| Bova172_1976 | Predicted | Polysaccharide lyase | PL26 | 1.94 | 1.5E−05 | −0.58 | 1.2E−01 | 0.45 | 3.6E−01 |
| | PUL 28 | | | | | | | | |
| Bova172_1977 | Predicted | Polysaccharide lyase | PL11_1 | −1.07 | 1.6E−05 | −0.31 | 2.0E−01 | −0.28 | 5.1E−01 |
| | PUL 28 | | | | | | | | |
| Bova172_1978 | Predicted | Cell surface glycan-binding | SusD | −0.06 | 8.9E−01 | −0.20 | 5.7E−01 | −0.42 | 4.1E−01 |
| | PUL 28 | lipoprotein, SusD family | | | | | | | |
| Bova172_1979 | Predicted | Outer membrane TonB-dependent | Trans | 0.88 | 3.2E−02 | 0.33 | 3.1E−01 | −0.13 | 8.1E−01 |
| | PUL 28 | transporter, SusC family | | | | | | | |
| Bova172_{—}1980 | Predicted | Cell surface glycan-binding | SusD | 1.18 | 2.7E−02 | 0.32 | 3.9E−01 | −0.04 | 9.5E−01 |
| | PUL 28 | lipoprotein, SusD family | | | | | | | |
| Bova172_{—}1981 | Predicted | Outer membrane TonB-dependent | Trans | 1.29 | 5.6E−03 | 0.46 | 1.9E−01 | −0.55 | 2.8E−01 |
| | PUL 28 | transporter, SusC family | | | | | | | |
| Bova172_1982 | Predicted | hypothetical protein | | 1.28 | 1.3E−02 | 0.19 | 6.4E−01 | −0.07 | 1.0E+00 |
| | PUL 28 | | | | | | | | |
| Bova172_1983 | Predicted | hypothetical protein | | 0.94 | 5.0E−02 | −0.75 | 4.3E−02 | 0.35 | 4.8E−01 |
| | PUL 28 | | | | | | | | |
| Bova172_1985 | Predicted | integrase | | 1.30 | 1.6E−05 | −0.02 | 9.4E−01 | 0.96 | 5.8E−02 |
| | PUL 28 | | | | | | | | |
| Bova172_1987 | Predicted | Outer membrane TonB-dependent | Trans | 0.10 | 3.9E−01 | 0.97 | 2.4E−33 | 0.56 | 2.6E−03 |
| | PUL 29 | transporter, SusC family | | | | | | | |
| Bova172_1988 | Predicted | Cell surface glycan-binding | SusD | 0.17 | 3.5E−01 | 1.76 | 4.7E−54 | −0.68 | 2.8E−02 |
| | PUL 29 | lipoprotein, SusD family | | | | | | | |
| Bova172_1989 | Predicted | hypothetical protein | | −0.93 | 2.3E−11 | 0.10 | 4.7E−01 | −0.40 | 1.7E−01 |
| | PUL 29 | | | | | | | | |
| Bova172_1990 | Predicted | homogalacturonan specific α- | GH28 | 3.36 | 1.5E−16 | 0.68 | 3.1E−02 | 0.58 | 2.2E−01 |
| | PUL 29 | galacturonidase/exo- | | | | | | | |
| | | polygalacturonase | | | | | | | |
| Bova172_1991 | Predicted | hypothetical protein | | 2.20 | 1.5E−05 | −0.28 | 5.0E−01 | −0.42 | 4.1E−01 |
| | PUL 29 | | | | | | | | |
| Bova172_{—}1992 | Predicted | β-D-glycosidase | GH42 | 2.68 | 1.7E−16 | 0.40 | 1.9E−01 | −0.15 | 7.7E−01 |
| | PUL 29 | | | | | | | | |
| Bova172_{—}1993 | Predicted | α-L/β-D-glycosidase | GH43_34- | 2.55 | 6.2E−17 | −0.71 | 3.7E−03 | −0.01 | 9.8E−01 |
| | PUL 29 | | GH43_18- | | | | | | |
| | | | CBM32 | | | | | | |
| Bova172_1994 | Predicted | β-galactosidase | GH2 | 2.94 | 1.8E−17 | 0.80 | 2.4E−02 | 0.88 | 9.0E−02 |
| | PUL 29 | | | | | | | | |
| Bova172_1995 | Predicted | rhamnogalacturonan | CE12 | 2.89 | 9.2E−06 | 0.86 | 9.7E−02 | 0.25 | 1.0E+00 |
| | PUL 29 | acetylesterase | | | | | | | |
| Bova172_{—}1996 | Predicted | RGI specific α-galacturonidase | GH28 | 2.06 | 1.3E−09 | −0.37 | 2.0E−01 | −0.26 | 5.6E−01 |
| | PUL 29 | | | | | | | | |
| Bova172_1997 | Predicted | hypothetical protein | | 0.31 | 2.6E−01 | −0.43 | 1.1E−01 | 0.73 | 8.7E−02 |
| | PUL 29 | | | | | | | | |
| Bova172_1998 | Predicted | RGI-disaccharide specific α- | GH28 | 0.64 | 1.1E−02 | 1.28 | 3.9E−06 | 0.10 | 8.4E−01 |
| | PUL 29 | galacturonidase | | | | | | | |
| Bova172_1999 | Predicted | rhamnogalacturonan α-L- | GH106 | 1.68 | 2.1E−14 | 2.11 | 2.4E−20 | 0.13 | 7.7E−01 |
| | PUL 29 | rhamnohydrolase/RGI specific | | | | | | | |
| | | alpha-L-rhamnosidase | | | | | | | |
| Bova172_2000 | Predicted | Cystathionine beta-lyase (EC | | 0.05 | 7.4E−01 | 0.27 | 8.9E−03 | 0.21 | 4.1E−01 |
| | PUL 29 | 4.4.1.8) | | | | | | | |
| Bova172_2001 | Predicted | hypothetical protein | | −2.80 | 1.1E−67 | 0.10 | 7.5E−01 | −2.05 | 1.9E−17 |
| | PUL 29 | | | | | | | | |
| Bova172_2002 | Predicted | Cell surface glycan-binding | SusD | −2.56 | 7.6E−76 | −0.28 | 2.4E−01 | −2.49 | 6.9E−30 |
| | PUL 29 | lipoprotein, SusD family | | | | | | | |
| Bova172_2003 | Predicted | Outer membrane TonB-dependent | Trans | −2.14 | 3.5E−165 | −0.77 | 1.5E−06 | −1.92 | 1.7E−87 |
| | PUL 29 | transporter, SusC family | | | | | | | |
| Bova172_2020 | Predicted | BaeS-type histidine kinase/ | REG | 0.41 | 3.9E−04 | −0.43 | 2.6E−05 | 0.76 | 1.1E−07 |
| | PUL 30 | OmpR-type DNA-binding response | | | | | | | |
| | | regulator | | | | | | | |
| Bova172_2021 | Predicted | SAM-dependent methyltransferase | | 0.48 | 4.0E−02 | −1.12 | 3.3E−09 | 1.75 | 1.2E−06 |
| | PUL 30 | | | | | | | | |
| Bova172_2022 | Predicted | hypothetical protein | | −0.31 | 5.9E−01 | −1.67 | 8.7E−03 | 0.47 | 1.0E+00 |
| | PUL 30 | | | | | | | | |
| Bova172_2023 | Predicted | beta-glucosidase (EC 3.2.1.21) | GH3 | −0.25 | 1.6E−01 | −0.33 | 1.3E−02 | 1.50 | 1.5E−11 |
| | PUL 30 | | | | | | | | |
| Bova172_2024 | Predicted | Polygalacturonase (EC 3.2.1.15) | GH28 | 0.41 | 3.9E−01 | 0.97 | 3.1E−02 | −0.04 | 9.5E−01 |
| | PUL 30 | | | | | | | | |
| Bova172_2025 | Predicted | hypothetical protein | CE8 | −0.29 | 4.9E−01 | 0.55 | 1.2E−01 | −0.06 | 9.2E−01 |
| | PUL 30 | | | | | | | | |
| Bova172_2026 | Predicted | hypothetical protein | | −0.03 | 9.5E−01 | 0.90 | 4.3E−02 | −0.46 | 1.0E+00 |
| | PUL 30 | | | | | | | | |
| Bova172_2027 | Predicted | hypothetical protein | | 0.04 | 9.5E−01 | 0.67 | 1.6E−01 | 0.44 | 1.0E+00 |
| | PUL 30 | | | | | | | | |
| Bova172_2028 | Predicted | Cell surface glycan-binding | SusD | −0.23 | 6.7E−01 | 0.51 | 2.0E−01 | 0.13 | 1.0E+00 |
| | PUL 30 | lipoprotein, SusD family | | | | | | | |
| Bova172_2029 | Predicted | Outer membrane TonB-dependent | Trans | 0.15 | 7.5E−01 | 1.25 | 1.9E−03 | −0.17 | 7.3E−01 |
| | PUL 30 | transporter, SusC family | | | | | | | |
| Bova172_2030 | Predicted | Pectate lyase (EC 4.2.2.2) | PL1_2 | −0.72 | 9.4E−02 | 0.13 | 7.2E−01 | −0.36 | 4.7E−01 |
| | PUL 30 | | | | | | | | |
| Bova172_2031 | Predicted | hypothetical protein | | −0.78 | 3.4E−02 | −0.06 | 8.8E−01 | 1.12 | 5.2E−02 |
| | PUL 30 | | | | | | | | |
| Bova172_2032 | Predicted | hypothetical protein | | −1.38 | 1.0E−03 | −1.15 | 3.2E−03 | 0.13 | 8.2E−01 |
| | PUL 30 | | | | | | | | |
| Bova172_2033 | Predicted | Pectate lyase (EC 4.2.2.2) | PL1_2 | 0.09 | 5.1E−01 | 0.06 | 4.9E−01 | −1.41 | 1.7E−06 |
| | PUL 30 | | | | | | | | |
| Bova172_2034 | Predicted | Two-component system response | | −1.51 | 2.4E−43 | −2.40 | 1.2E−165 | −3.59 | 1.1E−24 |
| | PUL 30 | regulator without neighboring | | | | | | | |
| | | kinase | | | | | | | |
| Bova172_2035 | Predicted | Transmembrane protein | | −1.71 | 7.3E−93 | −3.16 | 0.0E+00 | −3.70 | 1.1E−43 |
| | PUL 30 | | | | | | | | |
| Bova172_2036 | Predicted | FIG00409296: hypothetical protein | | −0.69 | 2.4E−01 | −1.11 | 1.0E−01 | 0.03 | 1.0E+00 |
| | PUL 30 | | | | | | | | |
| Bova172_2037 | Predicted | Outer membrane TonB-dependent | Trans | 3.27 | 2.7E−41 | 4.72 | 1.8E−71 | 1.74 | 1.5E−07 |
| | PUL 30 | transporter, SusC family | | | | | | | |
| Bova172_2038 | Predicted | Cell surface glycan-binding | SusD | 3.41 | 5.1E−44 | 4.99 | 5.3E−63 | 1.56 | 3.8E−05 |
| | PUL 30 | lipoprotein, SusD family | | | | | | | |
| Bova172_2039 | Predicted | hypothetical protein | | 4.91 | 9.8E−62 | 5.15 | 6.9E−46 | 1.39 | 2.2E−04 |
| | PUL 30 | | | | | | | | |
| Bova172_2040 | Predicted | hypothetical protein | CE8 | 5.99 | 1.1E−45 | 3.95 | 6.1E−22 | 2.02 | 9.1E−05 |
| | PUL 30 | | | | | | | | |
| Bova172_2041 | Predicted | Pectate lyase (EC 4.2.2.2) | PL1_2 | 6.38 | 2.0E−33 | 2.96 | 3.7E−10 | 1.34 | 1.2E−02 |
| | PUL 30 | | | | | | | | |
| Bova172_2042 | Predicted | Pectate lyase (EC 4.2.2.2) | PL1_2 | 6.06 | 1.3E−26 | 2.57 | 4.2E−07 | 1.10 | 5.2E−02 |
| | PUL 30 | | | | | | | | |
| Bova172_2043 | Predicted | Pectate lyase (EC 4.2.2.2) | PL1_2 | 3.00 | 4.0E−26 | 0.56 | 3.5E−02 | 1.33 | 1.0E−03 |
| | PUL 30 | | | | | | | | |
| Bova172_2044 | Predicted | BaeS-type histidine kinase/ | REG | −1.81 | 3.4E−183 | 0.17 | 1.7E−02 | −1.56 | 1.1E−52 |
| | PUL 30 | OmpR-type DNA-binding response | | | | | | | |
| | | regulator | | | | | | | |
| Bova172_2045 | Predicted | rhamnogalacturonan | CE8-CE12 | 1.58 | 3.2E−07 | 1.47 | 1.7E−06 | 1.21 | 5.8E−03 |
| | PUL 30 | acetylesterase | | | | | | | |
| Bova172_2046 | Predicted | hypothetical protein | | 0.23 | 7.0E−01 | −0.25 | 6.3E−01 | 0.02 | 1.0E+00 |
| | PUL 30 | | | | | | | | |
| Bova172_2047 | Predicted | rhamnogalacturonan | CE8 | 1.54 | 1.3E−06 | 1.29 | 7.8E−06 | 0.26 | 5.7E−01 |
| | PUL 30 | acetylesterase | | | | | | | |
| Bova172_2048 | Predicted | Rhamnogalacturonides | GH105 | 2.46 | 1.8E−36 | 0.24 | 2.2E−01 | 1.54 | 2.0E−09 |
| | PUL 30 | degradation protein RhiN | | | | | | | |
| Bova172_2061 | Predicted | hypothetical protein | CE | 0.12 | 8.2E−01 | 0.48 | 1.8E−01 | 0.12 | 8.3E−01 |
| | PUL 31 | | | | | | | | |
| Bova172_2062 | Predicted | FIG01423360: glycoside hydrolase | GH130 | −0.61 | 2.6E−01 | 1.72 | 5.4E−03 | −0.27 | 1.0E+00 |
| | PUL 31 | | | | | | | | |
| Bova172_2063 | Predicted | Putative alpha-1,2-mannosidase | GH92 | −0.48 | 3.7E−01 | 1.44 | 3.7E−03 | 0.49 | 1.0E+00 |
| | PUL 31 | | | | | | | | |
| Bova172_2064 | Predicted | Alpha-1,2-mannosidase | GH92 | 0.17 | 7.5E−01 | 0.26 | 5.4E−01 | −0.12 | 1.0E+00 |
| | PUL 31 | | | | | | | | |
| Bova172_2065 | Predicted | Sialic acid-specific 9-O- | | −1.05 | 1.1E−01 | −1.34 | 4.6E−02 | 0.03 | 1.0E+00 |
| | PUL 31 | acetylesterase | | | | | | | |
| Bova172_2066 | Predicted | hypothetical protein | | −0.15 | 7.7E−01 | −1.21 | 3.5E−05 | −0.36 | 4.5E−01 |
| | PUL 31 | | | | | | | | |
| Bova172_2067 | Predicted | Cell surface glycan-binding | SusD | −0.18 | 6.3E−01 | −2.26 | 4.1E−17 | −0.35 | 4.2E−01 |
| | PUL 31 | lipoprotein, SusD family | | | | | | | |
| Bova172_2068 | Predicted | hypothetical protein | | −2.02 | 1.8E−03 | −2.30 | 8.6E−05 | 0.13 | 1.0E+00 |
| | PUL 31 | | | | | | | | |
| Bova172_2069 | Predicted | hypothetical protein | | −0.82 | 1.1E−01 | −1.57 | 3.4E−06 | 0.42 | 1.0E+00 |
| | PUL 31 | | | | | | | | |
| Bova172_2070 | Predicted | Outer membrane TonB-dependent | | −0.53 | 2.3E−01 | −0.90 | 5.4E−04 | −0.89 | 1.3E−01 |
| | PUL 31 | transporter, SusC family | | | | | | | |
| Bova172_2071 | Predicted | Cell surface glycan-binding | SusD | −1.09 | 3.7E−02 | −0.17 | 6.9E−01 | 0.13 | 1.0E+00 |
| | PUL 31 | lipoprotein, SusD family | | | | | | | |
| Bova172_2072 | Predicted | Outer membrane TonB-dependent | Trans | 0.10 | 8.1E−01 | −0.24 | 3.8E−01 | 0.11 | 8.4E−01 |
| | PUL 31 | transporter, SusC family | | | | | | | |
| Bova172_2073 | Predicted | hypothetical protein | | 0.09 | 8.8E−01 | −0.39 | 5.0E−01 | 0.02 | 1.0E+00 |
| | PUL 31 | | | | | | | | |
| Bova172_2074 | Predicted | Alpha-glucosidase (EC 3.2.1.20) | GH31 | 0.83 | 5.9E−02 | −1.50 | 8.0E−08 | 0.04 | 9.5E−01 |
| | PUL 31 | | | | | | | | |
| Bova172_2075 | Predicted | hypothetical protein | | −0.38 | 5.0E−01 | −1.84 | 1.6E−02 | 0.03 | 1.0E+00 |
| | PUL 31 | | | | | | | | |
| Bova172_2076 | Predicted | alpha-rhamnosidase | GH78 | 0.25 | 6.6E−01 | 0.10 | 8.4E−01 | −0.19 | 1.0E+00 |
| | PUL 31 | | | | | | | | |
| Bova172_2077 | Predicted | alpha-rhamnosidase | | 0.09 | 8.7E−01 | 0.53 | 2.6E−01 | 0.55 | 1.0E+00 |
| | PUL 31 | | | | | | | | |
| Bova172_2078 | Predicted | hypothetical protein | CE | 0.10 | 8.5E−01 | −0.62 | 1.6E−01 | 0.03 | 1.0E+00 |
| | PUL 31 | | | | | | | | |
| Bova172_2079 | Predicted | Beta-mannosidase (EC 3.2.1.25) | GH2- | 0.33 | 4.5E−01 | 0.35 | 3.2E−01 | −0.14 | 7.9E−01 |
| | PUL 31 | | CBM32 | | | | | | |
| Bova172_2080 | Predicted | α-(1-4)-mannosidase | GH92 | 0.54 | 1.6E−01 | −0.88 | 8.2E−04 | −0.11 | 8.4E−01 |
| | PUL 31 | | | | | | | | |
| Bova172_{—}2081 | Predicted | alpha-mannosidase | GH38 | 1.21 | 1.2E−06 | −3.09 | 8.4E−47 | 0.28 | 5.5E−01 |
| | PUL 31 | | | | | | | | |
| Bova172_2082 | Predicted | hypothetical protein | | 1.60 | 8.0E−04 | −3.41 | 1.9E−10 | 0.30 | 5.5E−01 |
| | PUL 31 | | | | | | | | |
| Bova172_2083 | Predicted | RNA polymerase ECF-type sigma | REG | 0.06 | 9.1E−01 | −1.48 | 1.6E−03 | −0.06 | 1.0E+00 |
| | PUL 31 | factor | | | | | | | |
| Bova172_2084 | Predicted | hypothetical protein | | −1.04 | 1.1E−01 | −0.73 | 2.3E−01 | 0.03 | 1.0E+00 |
| | PUL 31 | | | | | | | | |
| Bova172_2085 | Predicted | Putative regulatory protein | REG | 0.16 | 2.0E−01 | −0.11 | 3.1E−01 | 0.66 | 2.9E−04 |
| | PUL 31 | | | | | | | | |
| Bova172_2164 | Predicted | RNA polymerase ECF-type sigma | REG | −0.30 | 2.9E−01 | 1.53 | 2.5E−11 | −0.24 | 6.3E−01 |
| | PUL 32 | factor | | | | | | | |
| Bova172_2165 | Predicted | Putative anti-sigma factor | REG | 0.36 | 1.1E−04 | −2.46 | 1.2E−226 | 0.23 | 2.5E−01 |
| | PUL 32 | | | | | | | | |
| Bova172_2166 | Predicted | Alpha-1,2-mannosidase | GH92 | 0.06 | 7.1E−01 | 0.32 | 5.1E−03 | −0.53 | 9.3E−02 |
| | PUL 32 | | | | | | | | |
| Bova172_2167 | Predicted | Alpha-1,2-mannosidase | GH92 | 0.13 | 6.1E−01 | 2.22 | 2.3E−38 | −1.03 | 1.5E−02 |
| | PUL 32 | | | | | | | | |
| Bova172_2168 | Predicted | hypothetical protein | | 0.46 | 2.2E−03 | −0.60 | 8.3E−06 | 1.01 | 5.7E−09 |
| | PUL 32 | | | | | | | | |
| Bova172_2169 | Predicted | Outer membrane TonB-dependent | Trans | −0.14 | 4.6E−01 | 0.59 | 2.6E−04 | −0.09 | 7.3E−01 |
| | PUL 32 | transporter, SusC family | | | | | | | |
| Bova172_2170 | Predicted | Cell surface glycan-binding | SusD | −0.69 | 1.0E−02 | 1.25 | 1.8E−07 | −0.68 | 4.6E−02 |
| | PUL 32 | lipoprotein, SusD family | | | | | | | |
| Bova172_2171 | Predicted | hypothetical protein | | −1.29 | 2.6E−04 | 1.03 | 1.2E−03 | −0.32 | 4.7E−01 |
| | PUL 32 | | | | | | | | |
| Bova172_2172 | Predicted | hypothetical protein | | −1.27 | 1.6E−03 | 1.46 | 5.4E−05 | −0.60 | 2.0E−01 |
| | PUL 32 | | | | | | | | |
| Bova172_2173 | Predicted | hypothetical protein | GH18 | −0.01 | 9.9E−01 | 1.10 | 6.2E−04 | −0.38 | 3.4E−01 |
| | PUL 32 | | | | | | | | |
| Bova172_2201 | Predicted | Alpha-glucosidase (EC 3.2.1.20) | GH97 | −0.28 | 2.7E−02 | 0.69 | 1.7E−12 | −0.17 | 3.7E−01 |
| | PUL 33 | | | | | | | | |
| Bova172_2202 | Predicted | Alpha-1,2-mannosidase | GH43_34- | 0.81 | 1.0E−02 | 3.68 | 2.1E−64 | −0.14 | 7.9E−01 |
| | PUL 33 | | GH92 | | | | | | |
| Bova172_2203 | Predicted | putative secretory protein | | 0.74 | 1.3E−01 | 3.25 | 8.1E−24 | 0.13 | 8.1E−01 |
| | PUL 33 | | | | | | | | |
| Bova172_2204 | Predicted | Alpha-1,2-mannosidase | GH92 | 0.94 | 2.0E−03 | 3.57 | 8.8E−62 | −0.31 | 5.0E−01 |
| | PUL 33 | | | | | | | | |
| Bova172_2205 | Predicted | Alpha-1,2-mannosidase | GH92 | 0.32 | 2.0E−01 | 3.09 | 1.0E−65 | −0.14 | 7.6E−01 |
| | PUL 33 | | | | | | | | |
| Bova172_2206 | Predicted | Maltodextrin glucosidase (EC | GH31 | −0.03 | 8.7E−01 | 1.57 | 1.5E−29 | 0.82 | 8.1E−03 |
| | PUL 33 | 3.2.1.20) | | | | | | | |
| Bova172_2207 | Predicted | hypothetical protein | | 0.43 | 4.0E−01 | 2.02 | 9.4E−15 | 0.12 | 8.4E−01 |
| | PUL 33 | | | | | | | | |
| Bova172_2208 | Predicted | hypothetical protein | | 0.80 | 9.9E−02 | 2.30 | 3.0E−27 | 0.12 | 8.3E−01 |
| | PUL 33 | | | | | | | | |
| Bova172_2209 | Predicted | Cell surface glycan-binding | SusD | 0.56 | 2.3E−01 | 2.35 | 6.3E−26 | −0.51 | 2.9E−01 |
| | PUL 33 | lipoprotein, SusD family | | | | | | | |
| Bova172_2210 | Predicted | Outer membrane TonB-dependent | Trans | 0.89 | 3.2E−02 | 2.02 | 3.2E−49 | −1.89 | 2.9E−04 |
| | PUL 33 | transporter, SusC family | | | | | | | |
| Bova172_2211 | Predicted | hypothetical protein | Pept | 0.34 | 4.2E−01 | 0.63 | 1.4E−05 | −0.55 | 2.4E−01 |
| | PUL 33 | | | | | | | | |
| Bova172_2212 | Predicted | putative alpha-1,6-mannanase | GH76 | 1.43 | 1.7E−02 | 2.19 | 2.1E−23 | −0.65 | 1.8E−01 |
| | PUL 33 | | | | | | | | |
| Bova172_2213 | Predicted | Alpha-glucosidase (EC 3.2.1.20) | GH31 | 0.76 | 9.4E−02 | 0.95 | 8.6E−09 | −0.11 | 8.4E−01 |
| | PUL 33 | | | | | | | | |
| Bova172_2214 | Predicted | DNA-binding response regulator, | REG | 2.25 | 1.4E−15 | 0.23 | 8.4E−02 | 1.45 | 1.7E−05 |
| | PUL 33 | AraC family | | | | | | | |
| Bova172_2223 | Predicted | Cell surface glycan-binding | SusD | 0.61 | 1.0E−02 | 1.84 | 6.3E−17 | 0.14 | 7.7E−01 |
| | PUL 34 | lipoprotein, SusD family | | | | | | | |
| Bova172_2224 | Predicted | Outer membrane TonB-dependent | Trans | 0.52 | 3.4E−05 | 1.50 | 6.7E−26 | 0.44 | 9.6E−02 |
| | PUL 34 | transporter, SusC family | | | | | | | |
| Bova172_2250 | Predicted | hypothetical protein | | 0.07 | 6.6E−01 | −1.15 | 3.3E−33 | −0.10 | 7.3E−01 |
| | PUL 35 | | | | | | | | |
| Bova172_2251 | Predicted | hypothetical protein | | −0.76 | 2.8E−04 | −0.38 | 4.1E−02 | −0.28 | 4.2E−01 |
| | PUL 35 | | | | | | | | |
| Bova172_2252 | Predicted | Cell surface glycan-binding | SusD | 0.27 | 4.9E−01 | 0.35 | 2.6E−01 | −0.02 | 9.6E−01 |
| | PUL 35 | lipoprotein, SusD family | | | | | | | |
| Bova172_2253 | Predicted | Outer membrane TonB-dependent | Trans | 0.50 | 4.4E−04 | −2.59 | 1.2E−122 | −0.33 | 2.7E−01 |
| | PUL 35 | transporter, SusC family | | | | | | | |
| Bova172_2254 | Predicted | Putative anti-sigma factor | REG | 0.14 | 2.1E−01 | −4.22 | 7.1E−286 | 0.03 | 9.3E−01 |
| | PUL 35 | | | | | | | | |
| Bova172_2255 | Predicted | RNA polymerase ECF-type sigma | REG | −0.96 | 2.6E−10 | −1.79 | 8.4E−37 | 0.11 | 7.3E−01 |
| | PUL 35 | factor | | | | | | | |
| Bova172_2281 | Predicted | hypothetical protein | | 0.21 | 5.9E−01 | −0.27 | 3.7E−01 | −0.09 | 8.5E−01 |
| | PUL 36 | | | | | | | | |
| Bova172_2282 | Predicted | hypothetical protein | | 0.44 | 2.6E−01 | −0.89 | 2.2E−03 | −0.63 | 1.1E−01 |
| | PUL 36 | | | | | | | | |
| Bova172_2283 | Predicted | Alpha-N-acetylglucosaminidase | GH89 | 0.18 | 5.7E−01 | −1.45 | 3.0E−10 | −0.66 | 6.6E−02 |
| | PUL 36 | (EC 3.2.1.50) | | | | | | | |
| Bova172_2284 | Predicted | hypothetical protein | | −0.34 | 2.2E−01 | −1.64 | 1.0E−10 | −0.84 | 3.8E−02 |
| | PUL 36 | | | | | | | | |
| Bova172_2285 | Predicted | Cell surface glycan-binding | SusD | 0.51 | 1.0E−01 | −1.78 | 4.8E−13 | −1.23 | 7.2E−04 |
| | PUL 36 | lipoprotein, SusD family | | | | | | | |
| Bova172_2286 | Predicted | Outer membrane TonB-dependent | Trans | 0.33 | 1.6E−02 | −3.92 | 2.3E−145 | −0.73 | 2.0E−04 |
| | PUL 36 | transporter, SusC family | | | | | | | |
| Bova172_2287 | Predicted | Putative anti-sigma factor | REG | −0.24 | 7.1E−03 | −4.99 | 0.0E+00 | −0.18 | 1.8E−01 |
| | PUL 36 | | | | | | | | |
| Bova172_2288 | Predicted | RNA polymerase ECF-type sigma | REG | −1.02 | 6.3E−11 | −1.01 | 2.5E−12 | 0.01 | 9.7E−01 |
| | PUL 36 | factor | | | | | | | |
| Bova172_2319 | Predicted | Cell surface glycan-binding | SusD | 0.72 | 1.7E−01 | 0.12 | 7.9E−01 | 0.04 | 1.0E+00 |
| | PUL 37 | lipoprotein, SusD family | | | | | | | |
| Bova172_2320 | Predicted | Outer membrane TonB-dependent | Trans | 0.51 | 3.1E−01 | 1.13 | 9.9E−03 | 0.24 | 1.0E+00 |
| | PUL 37 | transporter, SusC family | | | | | | | |
| Bova172_2321 | Predicted | Oligo alginate lyase | PL15 | 1.46 | 7.6E−03 | −0.01 | 9.8E−01 | 1.18 | 3.6E−02 |
| | PUL 37 | | | | | | | | |
| Bova172_2400 | Predicted | hypothetical protein | | 0.55 | 1.4E−01 | 0.87 | 2.7E−03 | 1.08 | 1.5E−02 |
| | PUL 38 | | | | | | | | |
| Bova172_2401 | Predicted | hypothetical protein | | 1.75 | 4.6E−03 | 1.92 | 4.1E−15 | −1.24 | 1.3E−02 |
| | PUL 38 | | | | | | | | |
| Bova172_2402 | Predicted | Probable zinc protease pqqL (EC | Pept | 0.39 | 2.7E−01 | 1.64 | 2.2E−12 | −0.34 | 4.3E−01 |
| | PUL 38 | 3.4.99.—) | | | | | | | |
| Bova172_2403 | Predicted | hypothetical protein | | −0.59 | 3.1E−01 | 0.46 | 3.2E−01 | −0.29 | 1.0E+00 |
| | PUL 38 | | | | | | | | |
| Bova172_2404 | Predicted | Cell surface glycan-binding | SusD | −0.24 | 5.9E−01 | 0.77 | 1.3E−02 | −0.80 | 1.4E−01 |
| | PUL 38 | lipoprotein, SusD family | | | | | | | |
| Bova172_2405 | Predicted | Outer membrane TonB-dependent | Trans | 0.10 | 6.9E−01 | 0.17 | 2.7E−01 | −0.54 | 1.0E−01 |
| | PUL 38 | transporter, SusC family | | | | | | | |
| Bova172_2406 | Predicted | Putative anti-sigma factor | REG | −0.54 | 1.3E−02 | −1.05 | 3.0E−13 | −0.74 | 3.6E−02 |
| | PUL 38 | | | | | | | | |
| Bova172_2407 | Predicted | RNA polymerase ECF-type sigma | REG | 0.02 | 9.4E−01 | −0.42 | 1.4E−02 | 0.31 | 4.2E−01 |
| | PUL 38 | factor | | | | | | | |
| Bova172_2415 | Predicted | hypothetical protein | | 2.20 | 1.7E−33 | 2.92 | 1.0E−136 | 0.08 | 8.3E−01 |
| | PUL 39 | | | | | | | | |
| Bova172_2416 | Predicted | Cell surface glycan-binding | SusD | 1.51 | 2.9E−08 | 3.90 | 5.7E−130 | −0.39 | 3.8E−01 |
| | PUL 39 | lipoprotein, SusD family | | | | | | | |
| Bova172_2417 | Predicted | Outer membrane TonB-dependent | Trans | 1.18 | 3.4E−11 | 3.72 | 1.7E−233 | −0.96 | 1.6E−02 |
| | PUL 39 | transporter, SusC family | | | | | | | |
| Bova172_2418 | Predicted | hydrolase (secreted protein) | GH76 | 1.24 | 2.7E−08 | 3.39 | 3.7E−141 | −0.35 | 4.6E−01 |
| | PUL 39 | | | | | | | | |
| Bova172_2419 | Predicted | hypothetical protein | | 0.44 | 6.0E−02 | 3.19 | 1.3E−103 | −2.15 | 1.9E−04 |
| | PUL 39 | | | | | | | | |
| Bova172_2420 | Predicted | hypothetical protein | | 1.54 | 4.8E−19 | 2.59 | 4.7E−90 | −1.22 | 2.5E−04 |
| | PUL 39 | | | | | | | | |
| Bova172_2421 | Predicted | DNA-binding response regulator, | REG | −0.79 | 2.7E−07 | 1.74 | 3.1E−46 | 0.32 | 1.9E−01 |
| | PUL 39 | AraC family | | | | | | | |
| Bova172_2422 | Predicted | Alpha-1,2-mannosidase | GH92 | −1.37 | 1.1E−16 | 3.17 | 5.5E−137 | −1.86 | 1.6E−05 |
| | PUL 39 | | | | | | | | |
| Bova172_2423 | Predicted | Bacillopeptidase F precursor (EC | | 0.33 | 5.6E−02 | 1.69 | 2.8E−49 | 0.06 | 8.8E−01 |
| | PUL 39 | 3.4.21.—) | | | | | | | |
| Bova172_2424 | Predicted | Putative hydrolase | GH76 | −1.48 | 1.2E−42 | 0.65 | 1.8E−11 | −1.19 | 1.8E−12 |
| | PUL 39 | | | | | | | | |
| Bova172_2425 | Predicted | hypothetical protein | GH125 | 0.14 | 6.6E−01 | 3.33 | 7.8E−56 | −0.32 | 4.9E−01 |
| | PUL 39 | | | | | | | | |
| Bova172_2426 | Predicted | hypothetical protein | | −0.19 | 7.4E−01 | 1.96 | 2.4E−05 | 0.14 | 1.0E+00 |
| | PUL 39 | | | | | | | | |
| Bova172_2427 | Predicted | FIG01423360: glycoside hydrolase | GH130 | 0.46 | 2.9E−01 | 4.15 | 2.3E−37 | −0.68 | 1.8E−01 |
| | PUL 39 | | | | | | | | |
| Bova172_2428 | Predicted | Alpha-1,2-mannosidase | GH92 | 0.48 | 4.8E−03 | 3.16 | 6.4E−139 | 0.02 | 9.5E−01 |
| | PUL 39 | | | | | | | | |
| Bova172_2432 | Predicted | acetyl xylan esterase A | CE6 | 1.15 | 9.2E−46 | −2.71 | 1.8E−273 | 1.19 | 1.1E−33 |
| | PUL 40 | | | | | | | | |
| Bova172_2433 | Predicted | beta-galactosidase (EC 3.2.1.23) | CBM57- | 0.70 | 2.5E−07 | −0.32 | 1.4E−02 | 0.97 | 1.1E−10 |
| | PUL 40 | | GH2 | | | | | | |
| Bova172_2434 | Predicted | alpha-xylosidase (EC 3.2.1.177) | GH31 | 0.26 | 4.6E−01 | 1.68 | 3.2E−11 | −0.02 | 9.7E−01 |
| | PUL 40 | | | | | | | | |
| Bova172_2435 | Predicted | hypothetical protein | GH | 0.78 | 6.8E−04 | −0.89 | 1.4E−07 | 0.75 | 2.9E−02 |
| | PUL 40 | | | | | | | | |
| Bova172_2436 | Predicted | hypothetical protein | | 1.18 | 8.4E−09 | −0.98 | 3.1E−11 | 1.06 | 6.3E−05 |
| | PUL 40 | | | | | | | | |
| Bova172_2437 | Predicted | hypothetical protein | GH50 | 0.30 | 5.4E−01 | 1.55 | 1.1E−04 | −0.31 | 5.2E−01 |
| | PUL 40 | | | | | | | | |
| Bova172_2438 | Predicted | hypothetical protein | | −0.03 | 9.5E−01 | 1.53 | 1.0E−04 | −0.17 | 7.5E−01 |
| | PUL 40 | | | | | | | | |
| Bova172_2439 | Predicted | hypothetical protein | | −0.31 | 5.9E−01 | 1.07 | 8.9E−02 | 0.25 | 1.0E+00 |
| | PUL 40 | | | | | | | | |
| Bova172_2440 | Predicted | Cell surface glycan-binding | SusD | −0.93 | 5.1E−02 | 1.54 | 1.4E−03 | 0.32 | 1.0E+00 |
| | PUL 40 | lipoprotein, SusD family | | | | | | | |
| Bova172_2441 | Predicted | Outer membrane TonB-dependent | Trans | −0.46 | 1.9E−01 | 1.25 | 3.4E−05 | −0.09 | 8.4E−01 |
| | PUL 40 | transporter, SusC family | | | | | | | |
| Bova172_2442 | Predicted | BaeS-type histidine kinase/ | REG | 1.03 | 1.1E−19 | 0.22 | 2.8E−02 | 1.06 | 7.7E−12 |
| | PUL 40 | OmpR-type DNA-binding response | | | | | | | |
| | | regulator | | | | | | | |
| Bova172_2449 | Predicted | hypothetical protein | | −1.83 | 5.9E−52 | −1.97 | 3.3E−71 | −2.57 | 9.6E−15 |
| | PUL 41 | | | | | | | | |
| Bova172_2450 | Predicted | hypothetical protein | | −2.91 | 1.4E−181 | −3.06 | 3.8E−191 | −2.59 | 3.8E−33 |
| | PUL 41 | | | | | | | | |
| Bova172_2451 | Predicted | Cell surface glycan-binding | SusD | −2.78 | 2.6E−202 | −3.29 | 1.6E−231 | −3.10 | 3.5E−48 |
| | PUL 41 | lipoprotein, SusD family | | | | | | | |
| Bova172_2452 | Predicted | Outer membrane TonB-dependent | Trans | −2.22 | 1.0E−205 | 4.51 | 0.0E+00 | −2.83 | 1.9E−144 |
| | PUL 41 | transporter, SusC family | | | | | | | |
| Bova172_2453 | Predicted | putative anti-sigma factor | REG | −0.12 | 2.6E−02 | −5.89 | 0.0E+00 | −1.17 | 6.3E−62 |
| | PUL 41 | | | | | | | | |
| Bova172_2454 | Predicted | RNA polymerase ECF-type sigma | REG | 0.56 | 1.7E−11 | −2.26 | 7.7E−191 | 1.26 | 6.2E−27 |
| | PUL 41 | factor | | | | | | | |
| Bova172_{—}2464 | Predicted | Polygalacturonase (EC 3.2.1.15) | GH28 | 1.22 | 1.4E−02 | −0.18 | 7.0E−01 | −0.04 | 9.5E−01 |
| | PUL 42 | | | | | | | | |
| Bova172_2465 | Predicted | BaeS-type histidine kinase/ | REG | −0.15 | 2.3E−01 | −1.76 | 1.4E−48 | −0.33 | 4.9E−02 |
| | PUL 42 | OmpR-type DNA-binding response | | | | | | | |
| | | regulator | | | | | | | |
| Bova172_2466 | Predicted | hypothetical protein | CE8 | 1.45 | 6.0E−04 | −0.65 | 2.0E−02 | −0.04 | 9.4E−01 |
| | PUL 42 | | | | | | | | |
| Bova172_2467 | Predicted | hypothetical protein | | 0.61 | 1.3E−01 | −0.62 | 2.8E−02 | 0.43 | 3.6E−01 |
| | PUL 42 | | | | | | | | |
| Bova172_2468 | Predicted | Cell surface glycan-binding | SusD | 0.34 | 4.4E−01 | −0.08 | 8.1E−01 | 0.11 | 8.4E−01 |
| | PUL 42 | lipoprotein, SusD family | | | | | | | |
| Bova172_{—}2469 | Predicted | Outer membrane TonB-dependent | Trans | 1.02 | 4.7E−04 | −1.13 | 3.5E−09 | 0.37 | 3.2E−01 |
| | PUL 42 | transporter, SusC family | | | | | | | |
| Bova172_2470 | Predicted | α-galactosidase | GH95 | 0.35 | 3.8E−01 | 0.68 | 4.7E−02 | 0.81 | 8.5E−02 |
| | PUL 42 | | | | | | | | |
| Bova172_2471 | Predicted | hypothetical protein | CE8 | 0.66 | 1.7E−01 | 0.14 | 7.1E−01 | 1.28 | 3.3E−02 |
| | PUL 42 | | | | | | | | |
| Bova172_2472 | Predicted | hypothetical protein | | −1.04 | 1.1E−01 | −0.73 | 2.3E−01 | 0.03 | 1.0E+00 |
| | PUL 42 | | | | | | | | |
| Bova172_2473 | Predicted | Pectate lyase (EC 4.2.2.2) | PL1_2 | −0.43 | 2.7E−01 | −0.99 | 4.9E−04 | −0.08 | 8.8E−01 |
| | PUL 42 | | | | | | | | |
| Bova172_2479 | Predicted | hypothetical protein | CE7 | 1.06 | 1.7E−02 | 0.39 | 2.5E−01 | 0.39 | 4.2E−01 |
| | PUL 43 | | | | | | | | |
| Bova172_2480 | Predicted | hypothetical protein | | −0.60 | 1.6E−01 | 0.40 | 3.1E−01 | 0.37 | 4.6E−01 |
| | PUL 43 | | | | | | | | |
| Bova172_2481 | Predicted | hypothetical protein | | 0.34 | 4.1E−01 | −0.07 | 8.4E−01 | 0.53 | 3.0E−01 |
| | PUL 43 | | | | | | | | |
| Bova172_2482 | Predicted | Cell surface glycan-binding | SusD | −0.08 | 8.8E−01 | −0.43 | 2.3E−01 | 0.12 | 8.2E−01 |
| | PUL 43 | lipoprotein, SusD family | | | | | | | |
| Bova172_2483 | Predicted | Outer membrane TonB-dependent | Trans | −0.03 | 9.4E−01 | 0.60 | 6.0E−02 | −0.33 | 4.8E−01 |
| | PUL 43 | transporter, SusC family | | | | | | | |
| Bova172_2484 | Predicted | hypothetical protein | | −0.84 | 1.7E−01 | −0.61 | 3.0E−01 | 0.02 | 1.0E+00 |
| | PUL 43 | | | | | | | | |
| Bova172_2485 | Predicted | RNA polymerase ECF-type sigma | REG | −1.86 | 8.0E−03 | −0.42 | 4.4E−01 | 0.03 | 1.0E+00 |
| | PUL 43 | factor | | | | | | | |
| Bova172_2486 | Predicted | hypothetical protein | | 0.36 | 4.5E−01 | −0.30 | 4.0E−01 | 0.41 | 4.1E−01 |
| | PUL 43 | | | | | | | | |
| Bova172_2487 | Predicted | BaeS-type histidine kinase/ | REG | 0.00 | 1.0E+00 | 0.01 | 9.8E−01 | 0.52 | 1.4E−02 |
| | PUL 43 | OmpR-type DNA-binding response | | | | | | | |
| | | regulator | | | | | | | |
| Bova172_2488 | Predicted | hypothetical protein | PL | 4.22 | 1.1E−23 | −2.23 | 4.2E−19 | 2.01 | 1.0E−07 |
| | PUL 43 | | | | | | | | |
| Bova172_2494 | Predicted | hypothetical protein | | −1.33 | 4.5E−10 | −1.13 | 2.0E−14 | −3.72 | 3.8E−12 |
| | PUL 44 | | | | | | | | |
| Bova172_2495 | Predicted | Endo-beta-N- | GH18 | −0.88 | 1.0E−03 | −0.98 | 7.4E−08 | −2.73 | 1.0E−08 |
| | PUL 44 | acetylglucosaminidase F2 | | | | | | | |
| Bova172_2496 | Predicted | Cell surface glycan-binding | SusD | −1.06 | 2.4E−08 | −1.34 | 2.0E−20 | −2.78 | 9.0E−14 |
| | PUL 44 | lipoprotein, SusD family | | | | | | | |
| Bova172_2497 | Predicted | Outer membrane TonB-dependent | Trans | 0.24 | 4.2E−02 | −2.21 | 7.6E−156 | −2.44 | 5.7E−43 |
| | PUL 44 | transporter, SusC family | | | | | | | |
| Bova172_{—}2498 | Predicted | Putative anti-sigma factor | REG | 1.10 | 3.5E−36 | −3.48 | 0.0E+00 | −0.55 | 7.6E−04 |
| | PUL 44 | | | | | | | | |
| Bova172_2499 | Predicted | RNA polymerase ECF-type sigma | REG | 0.34 | 3.9E−02 | −0.87 | 1.2E−08 | −0.01 | 9.9E−01 |
| | PUL 44 | factor | | | | | | | |
| Bova172_2532 | Predicted | Regulatory protein SusR | REG | −1.25 | 2.0E−29 | −0.23 | 1.1E−02 | −0.06 | 8.4E−01 |
| | PUL 45 | | | | | | | | |
| Bova172_2533 | Predicted | α-amylase (Neopullulanase) | GH13 | 2.20 | 1.6E−06 | 2.14 | 1.8E−08 | −0.93 | 6.5E−02 |
| | PUL 45 | | | | | | | | |
| Bova172_{—}2534 | Predicted | Glucan 1,4-α-glucosidase | GH97 | 1.07 | 6.8E−12 | 0.09 | 5.4E−01 | −0.23 | 3.7E−01 |
| | PUL 45 | | | | | | | | |
| Bova172_{—}2535 | Predicted | Outer membrane TonB-dependent | Trans | 1.55 | 5.3E−25 | −0.15 | 2.4E−01 | −1.20 | 6.1E−05 |
| | PUL 45 | transporter, SusC family | | | | | | | |
| Bova172_{—}2536 | Predicted | Cell surface glycan-binding | SusD | 1.61 | 6.8E−18 | 0.10 | 4.9E−01 | −1.32 | 1.3E−02 |
| | PUL 45 | lipoprotein, SusD family | | | | | | | |
| Bova172_{—}2537 | Predicted | outer membrane protein SusE | | 1.84 | 8.8E−15 | 0.01 | 9.8E−01 | −1.12 | 3.4E−02 |
| | PUL 45 | | | | | | | | |
| Bova172_2538 | Predicted | hypothetical protein | | 1.68 | 8.4E−22 | −0.23 | 1.1E−01 | 0.91 | 1.1E−02 |
| | PUL 45 | | | | | | | | |
| Bova172_2539 | Predicted | hypothetical protein | GH13- | 2.52 | 2.1E−26 | 0.30 | 9.2E−02 | 0.20 | 6.3E−01 |
| | PUL 45 | | CBM48 | | | | | | |
| Bova172_2540 | Predicted | UDP-2,3-diacylglucosamine | | −0.34 | 9.1E−04 | −0.47 | 3.3E−07 | 0.23 | 8.6E−02 |
| | PUL 46 | diphosphatase (EC 3.6.1.54) | | | | | | | |
| Bova172_2541 | Predicted | PaaD-like protein (DUF59) involved | | −0.78 | 6.8E−12 | −0.96 | 3.1E−19 | 0.40 | 5.4E−03 |
| | PUL 46 | in Fe—S cluster assembly | | | | | | | |
| Bova172_2542 | Predicted | beta-galactosidase (EC 3.2.1.23) | GH2 | −0.62 | 4.9E−05 | 0.40 | 2.6E−03 | 0.83 | 2.4E−03 |
| | PUL 46 | | | | | | | | |
| Bova172_2543 | Predicted | hypothetical protein | | 0.72 | 5.3E−02 | −0.17 | 5.4E−01 | −0.36 | 4.5E−01 |
| | PUL 46 | | | | | | | | |
| Bova172_2544 | Predicted | hypothetical protein | | 0.56 | 1.3E−01 | −0.23 | 4.1E−01 | −0.30 | 5.1E−01 |
| | PUL 46 | | | | | | | | |
| Bova172_2545 | Predicted | hypothetical protein | GH | 0.09 | 8.4E−01 | −0.48 | 1.2E−01 | 0.35 | 4.8E−01 |
| | PUL 46 | | | | | | | | |
| Bova172_2546 | Predicted | Cell surface glycan-binding | SusD | 0.31 | 5.0E−01 | 0.20 | 5.8E−01 | −0.36 | 4.6E−01 |
| | PUL 46 | lipoprotein, SusD family | | | | | | | |
| Bova172_2547 | Predicted | Outer membrane TonB-dependent | Trans | −0.10 | 7.8E−01 | −0.71 | 3.2E−03 | 0.39 | 3.9E−01 |
| | PUL 46 | transporter, SusC family | | | | | | | |
| Bova172_2548 | Predicted | hypothetical protein | | −1.20 | 7.3E−02 | −1.05 | 1.0E−01 | 0.14 | 1.0E+00 |
| | PUL 46 | | | | | | | | |
| Bova172_2549 | Predicted | Two-component system sensor | REG | −1.50 | 4.6E−39 | 0.60 | 9.3E−10 | −0.46 | 1.5E−02 |
| | PUL 46 | histidine kinase/response regulator | | | | | | | |
| | | hybrid | | | | | | | |
| Bova172_2550 | Predicted | UPF0758 family protein | | −0.82 | 1.5E−03 | 1.45 | 4.4E−12 | 0.78 | 8.6E−02 |
| | PUL 46 | | | | | | | | |
| Bova172_2551 | Predicted | beta-glycosyl hydrolase | GH20 | −0.24 | 5.7E−01 | 0.95 | 6.8E−03 | 0.30 | 5.0E−01 |
| | PUL 46 | | | | | | | | |
| Bova172_2559 | CAZyme | Rhamnogalacturonides | GH105 | 0.00 | 1.0E+00 | 0.81 | 2.6E−06 | −0.63 | 1.1E−01 |
| | cluster 4 | degradation protein RhiN | | | | | | | |
| Bova172_2560 | CAZyme | hypothetical protein | GH145 | 0.05 | 7.7E−01 | 0.10 | 5.2E−01 | −0.56 | 6.9E−02 |
| | cluster 4 | | | | | | | | |
| Bova172_2561 | CAZyme | beta-galactosidase (EC 3.2.1.23) | GH43_24 | 0.96 | 4.2E−11 | −0.44 | 6.4E−04 | 0.34 | 1.1E−01 |
| | cluster 4 | | | | | | | | |
| Bova172_2562 | CAZyme | hypothetical protein | | 0.41 | 2.3E−01 | 1.61 | 4.3E−08 | 0.22 | 6.5E−01 |
| | cluster 4 | | | | | | | | |
| Bova172_2563 | CAZyme | hypothetical protein | GH154 | −0.27 | 3.5E−01 | 0.54 | 1.7E−02 | 0.40 | 3.5E−01 |
| | cluster 4 | | | | | | | | |
| Bova172_2564 | CAZyme | Transcriptional regulator | REG | 0.46 | 3.1E−01 | 1.20 | 4.8E−03 | −0.10 | 8.5E−01 |
| | cluster 4 | | | | | | | | |
| Bova172_2565 | CAZyme | alpha-xylosidase (EC 3.2.1.177) | GH31 | 0.59 | 1.5E−01 | 0.13 | 7.0E−01 | −0.36 | 4.7E−01 |
| | cluster 4 | | | | | | | | |
| Bova172_2566 | CAZyme | TonB family protein | | −1.42 | 8.6E−29 | −3.37 | 1.5E−163 | 0.13 | 7.1E−01 |
| | cluster 4 | | | | | | | | |
| Bova172_2567 | CAZyme | Xylan 1,4-beta-xylosidase (EC | GH97 | −0.58 | 3.7E−08 | 0.03 | 8.2E−01 | 0.16 | 4.8E−01 |
| | cluster 4 | 3.2.1.37) | | | | | | | |
| Bova172_2568 | CAZyme | alpha-L-arabinofuranosidase (EC | GH51 | −0.03 | 8.4E−01 | 0.13 | 3.4E−01 | 0.05 | 8.2E−01 |
| | cluster 4 | 3.2.1.55) | | | | | | | |
| Bova172_2569 | CAZyme | Hypothetical glycoside hydrolase, | GH43_19 | −0.34 | 4.3E−01 | 1.91 | 5.3E−07 | 0.79 | 1.3E−01 |
| | cluster 4 | family 43, similar to arabinosidase | | | | | | | |
| Bova172_2570 | CAZyme | beta-galactosidase (EC 3.2.1.23) | CBM32- | −0.29 | 2.7E−01 | 0.94 | 4.3E−06 | −0.22 | 6.3E−01 |
| | cluster 4 | | GH35 | | | | | | |
| Bova172_2600 | Predicted | Sialic acid-specific 9-O- | | −0.24 | 5.2E−01 | 0.27 | 3.4E−01 | −0.49 | 3.0E−01 |
| | PUL 47 | acetylesterase | | | | | | | |
| Bova172_2601 | Predicted | hypothetical protein | GH115 | −0.11 | 7.5E−01 | 0.82 | 1.5E−03 | −0.41 | 4.0E−01 |
| | PUL 47 | | | | | | | | |
| Bova172_2602 | Predicted | hypothetical protein | | −0.48 | 3.2E−01 | −0.61 | 1.4E−01 | 0.44 | 1.0E+00 |
| | PUL 47 | | | | | | | | |
| Bova172_2603 | Predicted | hypothetical protein | | 0.33 | 4.3E−01 | 0.19 | 5.6E−01 | 0.52 | 3.2E−01 |
| | PUL 47 | | | | | | | | |
| Bova172_2604 | Predicted | hypothetical protein | | 0.35 | 4.8E−01 | −0.37 | 3.5E−01 | −0.87 | 1.0E+00 |
| | PUL 47 | | | | | | | | |
| Bova172_2605 | Predicted | hypothetical protein | SusD | −0.02 | 9.6E−01 | −0.29 | 3.9E−01 | 0.57 | 2.7E−01 |
| | PUL 47 | | | | | | | | |
| Bova172_2606 | Predicted | Outer membrane TonB-dependent | Trans | 0.72 | 8.4E−02 | −0.09 | 7.7E−01 | 0.62 | 2.2E−01 |
| | PUL 47 | transporter, SusC family | | | | | | | |
| Bova172_2607 | Predicted | hypothetical protein | GH30 | 0.83 | 4.6E−02 | −1.39 | 5.9E−06 | −0.42 | 3.8E−01 |
| | PUL 47 | | | | | | | | |
| Bova172_2608 | Predicted | BaeS-type histidine kinase/ | REG | −0.23 | 2.0E−02 | −1.01 | 9.0E−27 | 0.20 | 1.4E−01 |
| | PUL 47 | OmpR-type DNA-binding response | | | | | | | |
| | | regulator | | | | | | | |
| Bova172_2609 | Predicted | hypothetical protein | | 0.93 | 1.1E−04 | −2.10 | 8.0E−20 | 1.44 | 1.2E−08 |
| | PUL 47 | | | | | | | | |
| Bova172_2610 | Predicted | hypothetical protein | | −0.69 | 2.4E−01 | −1.11 | 1.0E−01 | 0.03 | 1.0E+00 |
| | PUL 47 | | | | | | | | |
| Bova172_2611 | Predicted | Putative large secreted protein | GH95 | −1.22 | 1.5E−26 | 0.80 | 1.8E−15 | −0.50 | 2.2E−02 |
| | PUL 47 | SCO0341 | | | | | | | |
| Bova172_2612 | Predicted | BaeS-type histidine kinase/ | REG | 1.18 | 2.0E−23 | 0.17 | 7.0E−02 | 1.06 | 3.8E−11 |
| | PUL 47 | OmpR-type DNA-binding response | | | | | | | |
| | | regulator | | | | | | | |
| Bova172_{—}2613 | Predicted | Endo-1,4-beta-xylanase (EC | GH43_29- | 1.58 | 1.6E−05 | −0.15 | 5.3E−01 | −0.30 | 5.4E−01 |
| | PUL 47 | 3.2.1.8) | CBM6 | | | | | | |
| Bova172_2614 | Predicted | Endo-1,4-beta-xylanase (EC | CE6-CE- | 1.42 | 1.3E−05 | −2.03 | 3.3E−21 | 0.74 | 9.7E−02 |
| | PUL 47 | 3.2.1.8) | CBM48 | | | | | | |
| Bova172_2615 | Predicted | | | 0.90 | 5.6E−05 | −2.28 | 8.2E−35 | 0.26 | 5.1E−01 |
| | PUL 47 | Chitin binding protein | GH5_21 | | | | | | |
| Bova172_2616 | Predicted | Endo-1,4-beta-xylanase (EC | GH10-CBM- | 0.31 | 2.6E−01 | −0.56 | 2.0E−02 | 0.27 | 5.3E−01 |
| | PUL 47 | 3.2.1.8) | CBM4-GH10 | | | | | | |
| Bova172_2617 | Predicted | hypothetical protein | | −0.99 | 3.2E−02 | 0.05 | 9.1E−01 | 0.29 | 1.0E+00 |
| | PUL 47 | | | | | | | | |
| Bova172_2618 | Predicted | Cell surface glycan-binding | SusD | −0.31 | 5.4E−01 | 0.18 | 6.6E−01 | −0.07 | 1.0E+00 |
| | PUL 47 | lipoprotein, SusD family | | | | | | | |
| Bova172_2619 | Predicted | Outer membrane TonB-dependent | Trans | 0.00 | 1.0E+00 | 0.27 | 4.4E−01 | 0.11 | 8.4E−01 |
| | PUL 47 | transporter, SusC family | | | | | | | |
| Bova172_2620 | Predicted | Cell surface glycan-binding | SusD | 0.19 | 6.8E−01 | −1.05 | 2.9E−03 | 0.77 | 1.4E−01 |
| | PUL 47 | lipoprotein, SusD family | | | | | | | |
| Bova172_2621 | Predicted | Outer membrane TonB-dependent | Trans | 0.31 | 3.5E−01 | 0.05 | 8.6E−01 | −0.04 | 9.4E−01 |
| | PUL 47 | transporter, SusC family | | | | | | | |
| Bova172_2622 | Predicted | Xylan 1,4-beta-xylosidase (EC | GH43_12 | −0.45 | 3.4E−01 | 0.44 | 3.2E−01 | 0.04 | 1.0E+00 |
| | PUL 47 | 3.2.1.37) | | | | | | | |
| Bova172_2623 | Predicted | Hypothetical glycoside hydrolase, | GH43_29- | −0.11 | 8.2E−01 | −0.32 | 4.1E−01 | −0.31 | 5.4E−01 |
| | PUL 47 | family 43, similar to arabinosidase | CBM6 | | | | | | |
| Bova172_2624 | Predicted | Xylan 1,4-beta-xylosidase (EC | GH97 | −0.15 | 7.6E−01 | −0.59 | 1.5E−01 | −0.19 | 7.1E−01 |
| | PUL 47 | 3.2.1.37) | | | | | | | |
| Bova172_2625 | Predicted | alpha-xylosidase (EC 3.2.1.177) | GH31 | 0.64 | 2.3E−01 | 0.27 | 5.5E−01 | −0.36 | 1.0E+00 |
| | PUL 47 | | | | | | | | |
| Bova172_2626 | Predicted | Xylan 1,4-beta-xylosidase (EC | GH43_12 | −0.78 | 1.5E−01 | 0.20 | 6.6E−01 | 0.23 | 1.0E+00 |
| | PUL 47 | 3.2.1.37) | | | | | | | |
| Bova172_2627 | Predicted | beta-glucosidase (EC 3.2.1.21) | GH3 | 0.81 | 2.0E−08 | −0.13 | 2.9E−01 | 0.65 | 5.0E−03 |
| | PUL 47 | | | | | | | | |
| Bova172_2628 | Predicted | Endo-1,4-beta-xylanase (EC | GH43_10 | 0.18 | 7.2E−01 | 0.22 | 6.0E−01 | 0.12 | 8.3E−01 |
| | PUL 47 | 3.2.1.8) | | | | | | | |
| Bova172_2641 | Predicted | Outer membrane TonB-dependent | Trans | −0.95 | 4.2E−08 | −1.74 | 8.5E−24 | −0.47 | 1.2E−01 |
| | PUL 48 | transporter, SusC family | | | | | | | |
| Bova172_2642 | Predicted | Cell surface glycan-binding | SusD | −0.34 | 2.3E−02 | −2.73 | 3.2E−55 | 0.19 | 5.0E−01 |
| | PUL 48 | lipoprotein, SusD family | | | | | | | |
| Bova172_2643 | Predicted | hypothetical protein | | −0.35 | 6.4E−02 | −1.61 | 2.0E−23 | −0.11 | 7.9E−01 |
| | PUL 48 | | | | | | | | |
| Bova172_2644 | Predicted | beta-N-acetylglucosaminidase (EC | GH20 | 0.58 | 6.9E−02 | −0.78 | 4.7E−03 | 0.57 | 2.2E−01 |
| | PUL 48 | 3.2.1.52) | | | | | | | |
| Bova172_2645 | Predicted | Sucrose-6-phosphate hydrolase | GH32 | −0.41 | 4.0E−01 | 0.27 | 5.3E−01 | 0.22 | 1.0E+00 |
| | PUL 48 | (EC 3.2.1.B3) | | | | | | | |
| Bova172_2646 | Predicted | N-acetyl glucosamine transporter, | Trans | −0.23 | 6.6E−01 | 0.67 | 1.5E−01 | 0.14 | 1.0E+00 |
| | PUL 48 | NagP | | | | | | | |
| Bova172_2647 | Predicted | N-acetylglucosamine-6-phosphate | CE9 | −0.26 | 6.3E−01 | 1.15 | 4.2E−02 | −0.30 | 1.0E+00 |
| | PUL 48 | deacetylase (EC 3.5.1.25) | | | | | | | |
| Bova172_2648 | Predicted | Glucosamine-6-phosphate | | −0.15 | 7.8E−01 | 0.68 | 1.8E−01 | −0.36 | 1.0E+00 |
| | PUL 48 | deaminase (EC 3.5.99.6) | | | | | | | |
| Bova172_2649 | Predicted | Putative dehydrogenase | | 0.00 | 1.0E+00 | −1.10 | 1.4E−15 | 0.82 | 1.9E−05 |
| | PUL 48 | | | | | | | | |
| Bova172_2650 | Predicted | Putative oxidoreductase | | −0.46 | 1.3E−02 | 0.16 | 4.0E−01 | 0.19 | 5.4E−01 |
| | PUL 48 | | | | | | | | |
| Bova172_2651 | Predicted | hypothetical protein | | −0.55 | 7.8E−02 | 0.15 | 6.1E−01 | 0.22 | 6.4E−01 |
| | PUL 48 | | | | | | | | |
| Bova172_2652 | Predicted | putative hydrogenase | | −0.77 | 2.0E−04 | 0.55 | 1.4E−03 | 0.57 | 1.2E−01 |
| | PUL 48 | | | | | | | | |
| Bova172_2653 | Predicted | hypothetical protein | | −0.16 | 7.8E−01 | 0.45 | 2.7E−01 | 0.13 | 1.0E+00 |
| | PUL 48 | | | | | | | | |
| Bova172_2654 | Predicted | Myo-inositol 2-dehydrogenase (EC | | 0.00 | 1.0E+00 | −1.57 | 2.7E−78 | 1.52 | 3.0E−32 |
| | PUL 48 | 1.1.1.18) | | | | | | | |
| Bova172_2655 | Predicted | DNA topoisomerase IV subunit A | | −0.70 | 9.9E−35 | 0.03 | 5.6E−01 | −1.06 | 5.7E−15 |
| | PUL 48 | (EC 5.99.1.3) | | | | | | | |
| Bova172_2656 | Predicted | hypothetical protein | | −0.64 | 3.3E−05 | 1.25 | 6.5E−19 | −0.51 | 1.6E−01 |
| | PUL 48 | | | | | | | | |
| Bova172_2657 | Predicted | Carboxyl-terminal protease-related | Pept | −0.90 | 1.6E−06 | 2.72 | 2.0E−39 | −0.55 | 2.4E−01 |
| | PUL 48 | protein | | | | | | | |
| Bova172_2660 | Predicted | Outer membrane TonB-dependent | Trans | −1.53 | 6.0E−80 | 2.06 | 8.5E−103 | −0.65 | 6.6E−06 |
| | PUL 49 | transporter, SusC family | | | | | | | |
| Bova172_2661 | Predicted | Cell surface glycan-binding | SusD | 0.43 | 6.4E−03 | 1.90 | 1.9E−51 | −0.37 | 1.7E−01 |
| | PUL 49 | lipoprotein, SusD family | | | | | | | |
| Bova172_2662 | Predicted | beta-glucosidase (EC 3.2.1.21) | GH3 | 0.28 | 1.5E−01 | 2.99 | 2.3E−81 | −1.20 | 1.6E−05 |
| | PUL 49 | | | | | | | | |
| Bova172_2663 | Predicted | Periplasmic beta-glucosidase (EC | GH144 | 0.36 | 7.1E−02 | 3.18 | 2.0E−73 | −1.04 | 2.2E−03 |
| | PUL 49 | 3.2.1.21) | | | | | | | |
| Bova172_2704 | Predicted | hypothetical protein | GH125 | −0.66 | 2.2E−04 | −1.50 | 1.1E−26 | −1.88 | 1.5E−09 |
| | PUL 50 | | | | | | | | |
| Bova172_2705 | Predicted | Alpha-1,2-mannosidase | GH92 | −0.20 | 2.4E−01 | −0.99 | 3.8E−12 | −1.28 | 1.3E−08 |
| | PUL 50 | | | | | | | | |
| Bova172_2706 | Predicted | Glutaminase A | | −1.03 | 2.7E−10 | 0.34 | 5.7E−02 | −1.15 | 2.0E−07 |
| | PUL 50 | | | | | | | | |
| Bova172_2707 | Predicted | hypothetical protein | | −1.86 | 4.1E−12 | 1.90 | 6.1E−08 | −2.55 | 3.0E−12 |
| | PUL 50 | | | | | | | | |
| Bova172_2708 | Predicted | alpha-1,6-mannanase | GH76 | −1.39 | 1.3E−07 | 0.44 | 1.5E−01 | −1.49 | 1.6E−06 |
| | PUL 50 | | | | | | | | |
| Bova172_2709 | Predicted | hypothetical protein | | −1.40 | 3.9E−19 | 1.65 | 6.2E−11 | −3.40 | 1.2E−32 |
| | PUL 50 | | | | | | | | |
| Bova172_2710 | Predicted | hypothetical protein | | −0.99 | 2.6E−08 | 0.64 | 1.6E−02 | −3.38 | 1.1E−27 |
| | PUL 50 | | | | | | | | |
| Bova172_2711 | Predicted | alpha-1,6-mannanase | GH76 | −1.23 | 1.7E−11 | 0.70 | 1.6E−02 | −3.33 | 1.4E−32 |
| | PUL 50 | | | | | | | | |
| Bova172_2712 | Predicted | Cell surface glycan-binding | SusD | −1.65 | 7.5E−46 | 0.79 | 4.7E−04 | −3.45 | 3.7E−62 |
| | PUL 50 | lipoprotein, SusD family | | | | | | | |
| Bova172_2713 | Predicted | Outer membrane TonB-dependent | Trans | −2.14 | 9.8E−201 | −1.15 | 1.1E−31 | −2.91 | 3.7E−148 |
| | PUL 50 | transporter, SusC family | | | | | | | |
| Bova172_2714 | Predicted | Putative anti-sigma factor | REG | −0.73 | 2.2E−32 | −3.20 | 0.0E+00 | −1.98 | 2.2E−113 |
| | PUL 50 | | | | | | | | |
| Bova172_2715 | Predicted | RNA polymerase ECF-type sigma | REG | −0.15 | 5.1E−01 | 0.74 | 8.7E−07 | −0.66 | 7.2E−02 |
| | PUL 50 | factor | | | | | | | |
| Bova172_2716 | CAZyme | arabinan endo-1,5-alpha-L- | GH43_34 | −0.47 | 6.4E−06 | 0.39 | 3.8E−05 | 0.63 | 6.1E−04 |
| | cluster 5 | arabinosidase A precursor | | | | | | | |
| Bova172_2717 | CAZyme | alpha-L-arabinofuranosidase II (EC | GH43_26 | −0.56 | 1.6E−05 | −0.63 | 3.5E−10 | 0.10 | 7.7E−01 |
| | cluster 5 | 3.2.1.55) | | | | | | | |
| Bova172_2718 | CAZyme | hypothetical protein | | −0.83 | 1.6E−01 | −0.31 | 5.6E−01 | 0.02 | 1.0E+00 |
| | cluster 5 | | | | | | | | |
| Bova172_2719 | CAZyme | beta-galactosidase (EC 3.2.1.23) | GH2 | 0.00 | 1.0E+00 | 0.61 | 5.7E−04 | −0.16 | 6.8E−01 |
| | cluster 5 | | | | | | | | |
| Bova172_{—}2720 | CAZyme | Arylsulfatase (EC 3.1.6.1) | Sulf | 1.11 | 3.9E−02 | −0.35 | 4.8E−01 | 0.62 | 2.1E−01 |
| | cluster 5 | | | | | | | | |
| Bova172_2731 | Predicted | hypothetical protein | | 1.54 | 9.2E−04 | −0.56 | 8.7E−02 | 0.19 | 7.1E−01 |
| | PUL 51 | | | | | | | | |
| Bova172_2732 | Predicted | Cell surface glycan-binding | SusD | 0.93 | 2.9E−02 | −0.94 | 5.4E−04 | 0.22 | 6.5E−01 |
| | PUL 51 | lipoprotein, SusD family | | | | | | | |
| Bova172_2733 | Predicted | Outer membrane TonB-dependent | Trans | 0.24 | 4.9E−01 | −0.62 | 1.2E−02 | 0.10 | 8.4E−01 |
| | PUL 51 | transporter, SusC family | | | | | | | |
| Bova172_2734 | Predicted | hypothetical protein | | 0.52 | 2.6E−01 | −0.90 | 5.2E−03 | 0.79 | 1.0E−01 |
| | PUL 51 | | | | | | | | |
| Bova172_2735 | Predicted | Glutaminase A | | −0.23 | 6.9E−01 | −0.75 | 1.8E−01 | 0.02 | 1.0E+00 |
| | PUL 51 | | | | | | | | |
| Bova172_2736 | Predicted | Integrase | Int | −0.03 | 7.9E−01 | −0.29 | 2.6E−03 | 0.66 | 1.2E−04 |
| | PUL 51 | | | | | | | | |
| Bova172_2737 | Predicted | Glutaminase A | | 0.60 | 6.3E−02 | −1.24 | 5.0E−07 | 0.09 | 8.5E−01 |
| | PUL 51 | | | | | | | | |
| Bova172_2738 | Predicted | hypothetical protein | | −0.14 | 7.6E−01 | −0.65 | 5.2E−02 | −0.12 | 8.3E−01 |
| | PUL 51 | | | | | | | | |
| Bova172_2739 | Predicted | Outer membrane TonB-dependent | Trans | 0.59 | 1.2E−01 | −0.47 | 7.9E−02 | 0.04 | 9.5E−01 |
| | PUL 51 | transporter, SusC family | | | | | | | |
| Bova172_2740 | Predicted | Cell surface glycan-binding | SusD | 0.56 | 2.4E−01 | −0.56 | 1.2E−01 | −0.12 | 8.3E−01 |
| | PUL 51 | lipoprotein, SusD family | | | | | | | |
| Bova172_2741 | Predicted | hypothetical protein | | 0.40 | 4.2E−01 | −0.92 | 1.1E−02 | −0.47 | 3.5E−01 |
| | PUL 51 | | | | | | | | |
| Bova172_2743 | Predicted | Glutaminase A | | −0.72 | 2.2E−01 | −0.19 | 7.4E−01 | 0.36 | 1.0E+00 |
| | PUL 52 | | | | | | | | |
| Bova172_2744 | Predicted | Outer membrane TonB-dependent | Trans | −0.18 | 7.3E−01 | 0.42 | 3.3E−01 | −0.13 | 1.0E+00 |
| | PUL 52 | transporter, SusC family | | | | | | | |
| Bova172_2745 | Predicted | Cell surface glycan-binding | SusD | −0.87 | 1.2E−01 | 0.54 | 3.1E−01 | 0.22 | 1.0E+00 |
| | PUL 52 | lipoprotein, SusD family | | | | | | | |
| Bova172_2746 | Predicted | Outer membrane TonB-dependent | Trans | 0.00 | 1.0E+00 | 0.15 | 7.2E−01 | 0.39 | 4.4E−01 |
| | PUL 52 | transporter, SusC family | | | | | | | |
| Bova172_2747 | Predicted | Cell surface glycan-binding | SusD | 0.14 | 8.1E−01 | 0.31 | 5.0E−01 | −0.24 | 1.0E+00 |
| | PUL 52 | lipoprotein, SusD family | | | | | | | |
| Bova172_2748 | Predicted | hypothetical protein | | 0.24 | 6.5E−01 | 0.81 | 1.3E−01 | 0.51 | 1.0E+00 |
| | PUL 52 | | | | | | | | |
| Bova172_2749 | Predicted | Arylsulfatase (EC 3.1.6.1) | Sulf | −0.56 | 1.8E−01 | 0.51 | 1.2E−01 | 0.11 | 8.3E−01 |
| | PUL 52 | | | | | | | | |
| Bova172_2750 | Predicted | Arylsulfatase (EC 3.1.6.1) | Sulf | 0.53 | 1.7E−01 | 1.93 | 2.1E−09 | −0.03 | 9.6E−01 |
| | PUL 52 | | | | | | | | |
| Bova172_2751 | Predicted | BaeS-type histidine kinase/ | REG | 0.75 | 5.2E−06 | 3.44 | 1.3E−76 | −1.12 | 4.5E−04 |
| | PUL 52 | OmpR-type DNA-binding response | | | | | | | |
| | | regulator | | | | | | | |
| Bova172_2877 | CAZyme | Chondroitinase (chondroitin lyase) | PL8_2 | 0.29 | 5.2E−01 | 2.22 | 3.1E−12 | −0.73 | 1.6E−01 |
| | cluster 6 | | | | | | | | |
| Bova172_2878 | CAZyme | N-acetylgalactosamine 6-sulfate | Sulf | −1.24 | 1.7E−02 | 2.26 | 4.4E−08 | −0.14 | 7.9E−01 |
| | cluster 6 | sulfatase (GALNS) | | | | | | | |
| Bova172_2879 | CAZyme | Glucuronyl hydrolase | GH88 | −0.43 | 1.6E−01 | 1.13 | 5.7E−07 | 0.25 | 5.7E−01 |
| | cluster 6 | | | | | | | | |
| Bova172_2880 | Predicted | RNA polymerase ECF-type sigma | REG | 0.41 | 1.6E−01 | 1.48 | 7.2E−13 | 0.23 | 6.3E−01 |
| | PUL 53 | factor | | | | | | | |
| Bova172_2881 | Predicted | hypothetical protein | | 0.59 | 3.2E−01 | −3.25 | 8.5E−06 | 0.25 | 1.0E+00 |
| | PUL 53 | | | | | | | | |
| Bova172_2882 | Predicted | hypothetical protein | | 1.26 | 4.3E−41 | −5.65 | 0.0E+00 | −0.35 | 2.9E−02 |
| | PUL 53 | | | | | | | | |
| Bova172_2883 | Predicted | Putative anti-sigma factor | REG | 1.84 | 1.1E−112 | −6.69 | 0.0E+00 | 0.39 | 4.4E−05 |
| | PUL 53 | | | | | | | | |
| Bova172_{—}2884 | Predicted | Outer membrane TonB-dependent | Trans | 1.97 | 3.2E−114 | −3.81 | 0.0E+00 | −0.55 | 1.7E−04 |
| | PUL 53 | transporter, SusC family | | | | | | | |
| Bova172_{—}2885 | Predicted | Cell surface glycan-binding | SusD | 1.07 | 5.2E−12 | −2.40 | 1.2E−120 | −1.40 | 1.9E−06 |
| | PUL 53 | lipoprotein, SusD family | | | | | | | |
| Bova172_2889 | Predicted | hypothetical protein | GH106 | 1.87 | 5.9E−05 | 1.66 | 2.3E−10 | 0.31 | 5.1E−01 |
| | PUL 54 | | | | | | | | |
| Bova172_2890 | Predicted | beta-galactosidase (EC 3.2.1.23) | GH2 | 0.30 | 4.3E−01 | 0.20 | 4.5E−01 | −0.23 | 6.4E−01 |
| | PUL 54 | | | | | | | | |
| Bova172_2891 | Predicted | hypothetical protein | | −0.22 | 6.2E−01 | 0.07 | 8.3E−01 | −0.57 | 2.8E−01 |
| | PUL 54 | | | | | | | | |
| Bova172_2892 | Predicted | Arabinan endo-1,5-alpha-L- | GH43_3 | −0.02 | 9.7E−01 | 0.87 | 1.2E−02 | −0.47 | 3.5E−01 |
| | PUL 54 | arabinosidase (EC 3.2.1.99) | | | | | | | |
| Bova172_2893 | Predicted | Cell surface glycan-binding | SusD | −0.59 | 1.1E−01 | 0.18 | 6.1E−01 | −0.48 | 3.1E−01 |
| | PUL 54 | lipoprotein, SusD family | | | | | | | |
| Bova172_2894 | Predicted | Outer membrane TonB-dependent | Trans | −0.08 | 7.9E−01 | −0.58 | 2.2E−03 | −0.62 | 1.1E−01 |
| | PUL 54 | transporter, SusC family | | | | | | | |
| Bova172_2895 | Predicted | hypothetical protein | | 1.30 | 6.1E−10 | −2.18 | 4.6E−46 | 0.01 | 9.6E−01 |
| | PUL 54 | | | | | | | | |
| Bova172_2896 | Predicted | RNA polymerase ECF-type sigma | REG | −1.54 | 4.9E−14 | −0.99 | 2.2E−08 | 0.29 | 4.2E−01 |
| | PUL 54 | factor | | | | | | | |
| Bova172_2897 | Predicted | Putative anti-sigma factor | REG | −0.60 | 7.1E−05 | −1.67 | 1.6E−36 | −0.19 | 5.9E−01 |
| | PUL 54 | | | | | | | | |
| Bova172_2910 | Predicted | BaeS-type histidine kinase/ | REG | −0.84 | 3.1E−37 | 0.00 | 9.7E−01 | −0.45 | 3.0E−06 |
| | PUL 55 | OmpR-type DNA-binding response | | | | | | | |
| | | regulator | | | | | | | |
| Bova172_2911 | Predicted | Arylsulfatase (EC 3.1.6.1) | Sulf | 0.51 | 9.9E−02 | −0.29 | 2.5E−01 | 0.94 | 2.0E−02 |
| | PUL 55 | | | | | | | | |
| Bova172_2912 | Predicted | hypothetical protein | | −1.45 | 4.6E−02 | −1.03 | 1.0E−01 | 0.03 | 1.0E+00 |
| | PUL 55 | | | | | | | | |
| Bova172_2913 | Predicted | Outer membrane TonB-dependent | Trans | −0.17 | 5.0E−01 | 0.69 | 1.5E−03 | 0.22 | 5.8E−01 |
| | PUL 55 | transporter, SusC family | | | | | | | |
| Bova172_2914 | Predicted | Cell surface glycan-binding | SusD | −0.02 | 9.6E−01 | 1.08 | 3.6E−04 | −0.42 | 3.7E−01 |
| | PUL 55 | lipoprotein, SusD family | | | | | | | |
| Bova172_2915 | Predicted | hypothetical protein | | 0.55 | 1.9E−01 | 0.60 | 4.2E−02 | 0.23 | 6.4E−01 |
| | PUL 55 | | | | | | | | |
| Bova172_2916 | Predicted | tRNA-dihydrouridine synthase | | 0.15 | 5.3E−01 | 2.00 | 5.4E−27 | −0.02 | 9.8E−01 |
| | PUL 55 | BT3326 | | | | | | | |
| Bova172_2917 | Predicted | hypothetical protein | | −0.28 | 2.8E−01 | 3.71 | 1.9E−58 | −0.13 | 8.0E−01 |
| | PUL 55 | | | | | | | | |
| Bova172_2918 | Predicted | Chondroitinase (chondroitin lyase) | PL8_2 | 2.15 | 1.1E−09 | 1.34 | 3.4E−07 | 1.30 | 2.6E−03 |
| | PUL 55 | | | | | | | | |
| Bova172_2919 | Predicted | hypothetical protein | | 1.95 | 2.1E−04 | 0.34 | 4.2E−01 | 1.81 | 1.1E−02 |
| | PUL 55 | | | | | | | | |
| Bova172_2942 | Predicted | beta-glucosidase (EC 3.2.1.21) | GH3 | 0.33 | 2.7E−10 | −4.04 | 0.0E+00 | −0.35 | 1.5E−06 |
| | PUL 56 | | | | | | | | |
| Bova172_2943 | Predicted | hypothetical protein | | 1.25 | 4.7E−13 | 0.06 | 6.7E−01 | −1.16 | 3.1E−07 |
| | PUL 56 | | | | | | | | |
| Bova172_2944 | Predicted | glycoside hydrolase family 30, | GH30_3 | 0.34 | 1.3E−01 | 1.88 | 6.6E−18 | −1.76 | 1.9E−06 |
| | PUL 56 | candidate beta-glycosidase | | | | | | | |
| Bova172_2945 | Predicted | Cell surface glycan-binding | SusD | −0.34 | 1.4E−01 | 1.71 | 1.2E−13 | −2.05 | 9.8E−08 |
| | PUL 56 | lipoprotein, SusD family | | | | | | | |
| Bova172_2946 | Predicted | Outer membrane TonB-dependent | Trans | −2.15 | 1.9E−41 | 1.46 | 4.2E−17 | −2.04 | 1.2E−17 |
| | PUL 56 | transporter, SusC family | | | | | | | |
| Bova172_2947 | Predicted | Regulatory protein SusR | REG | 0.37 | 2.6E−02 | −0.35 | 7.1E−03 | 0.52 | 3.3E−02 |
| | PUL 56 | | | | | | | | |
| Bova172_2954 | Predicted | RNA polymerase ECF-type sigma | REG | −1.02 | 3.6E−06 | 0.04 | 8.5E−01 | 0.00 | 1.0E+00 |
| | PUL 57 | factor | | | | | | | |
| Bova172_{—}2955 | Predicted | putative anti-sigma factor | REG | 1.35 | 3.0E−40 | −6.11 | 0.0E+00 | 0.01 | 9.7E−01 |
| | PUL 57 | | | | | | | | |
| Bova172_2956 | Predicted | Outer membrane TonB-dependent | Trans | −1.49 | 1.4E−75 | −4.20 | 3.0E−288 | −2.56 | 6.8E−105 |
| | PUL 57 | transporter, SusC family | | | | | | | |
| Bova172_2957 | Predicted | Cell surface glycan-binding | SusD | −3.02 | 8.9E−69 | −3.12 | 1.1E−44 | −2.84 | 1.4E−17 |
| | PUL 57 | lipoprotein, SusD family | | | | | | | |
| Bova172_2958 | Predicted | hypothetical protein | | −2.60 | 1.1E−75 | −3.18 | 1.6E−72 | −2.50 | 8.9E−16 |
| | PUL 57 | | | | | | | | |
| Bova172_2959 | Predicted | Glycerophosphoryl diester | | −2.68 | 1.0E−37 | −2.76 | 1.1E−33 | −2.49 | 1.5E−06 |
| | PUL 57 | phosphodiesterase (EC 3.1.4.46) | | | | | | | |
| Bova172_2960 | Predicted | Glycerophosphoryl diester | | −1.97 | 9.3E−22 | −2.40 | 4.4E−26 | −1.87 | 3.2E−05 |
| | PUL 57 | phosphodiesterase (EC 3.1.4.46) | | | | | | | |
| Bova172_2968 | Predicted | Regulatory protein SusR | REG | −0.02 | 8.6E−01 | −0.30 | 1.1E−03 | 0.33 | 5.9E−02 |
| | PUL 58 | | | | | | | | |
| Bova172_2969 | Predicted | Sulfatase | Sulf | 0.15 | 6.4E−01 | 1.66 | 1.9E−11 | 0.05 | 9.4E−01 |
| | PUL 58 | | | | | | | | |
| Bova172_2970 | Predicted | Arylsulfatase (EC 3.1.6.1) | Sulf | −0.68 | 8.5E−03 | 1.71 | 5.7E−12 | −0.22 | 6.5E−01 |
| | PUL 58 | | | | | | | | |
| Bova172_2971 | Predicted | Arylsulfatase (EC 3.1.6.1) | Sulf | −0.84 | 1.4E−04 | 1.20 | 7.7E−08 | 0.29 | 4.7E−01 |
| | PUL 58 | | | | | | | | |
| Bova172_2972 | Predicted | Outer membrane TonB-dependent | Trans | −1.75 | 4.9E−26 | 0.35 | 9.1E−02 | −0.95 | 9.4E−04 |
| | PUL 58 | transporter, SusC family | | | | | | | |
| Bova172_2973 | Predicted | Cell surface glycan-binding | SusD | −2.54 | 1.1E−20 | 0.83 | 4.5E−03 | −1.16 | 1.4E−02 |
| | PUL 58 | lipoprotein, SusD family | | | | | | | |
| Bova172_2974 | Predicted | hypothetical protein | | −1.93 | 1.1E−11 | 0.43 | 1.3E−01 | −2.13 | 6.4E−04 |
| | PUL 58 | | | | | | | | |
| Bova172_2975 | Predicted | Arylsulfatase (EC 3.1.6.1) | Sulf | −1.31 | 4.7E−04 | 1.72 | 1.7E−05 | −0.47 | 3.5E−01 |
| | PUL 58 | | | | | | | | |
| Bova172_2976 | Predicted | Arylsulfatase (EC 3.1.6.1) | Sulf | 0.13 | 6.6E−01 | 0.07 | 7.5E−01 | 0.28 | 4.9E−01 |
| | PUL 58 | | | | | | | | |
| Bova172_2977 | Predicted | Arylsulfatase (EC 3.1.6.1) | Sulf | 0.97 | 2.1E−03 | 1.47 | 2.8E−07 | 0.37 | 3.6E−01 |
| | PUL 58 | | | | | | | | |
| Bova172_2978 | Predicted | Putative alpha-1,6-mannanase | GH76 | 0.25 | 6.1E−01 | 2.43 | 2.5E−09 | 0.04 | 9.5E−01 |
| | PUL 58 | | | | | | | | |
| Bova172_2979 | Predicted | hypothetical protein | GH125 | 0.03 | 9.5E−01 | 2.50 | 9.9E−16 | 0.48 | 3.0E−01 |
| | PUL 58 | | | | | | | | |
| Bova172_2992 | Predicted | hypothetical protein | | 0.40 | 3.5E−01 | 0.47 | 1.7E−01 | −0.17 | 7.3E−01 |
| | PUL 59 | | | | | | | | |
| Bova172_2993 | Predicted | hypothetical protein | Pept | 1.15 | 7.5E−04 | 0.44 | 9.2E−02 | −0.44 | 2.6E−01 |
| | PUL 59 | | | | | | | | |
| Bova172_2994 | Predicted | Dipeptidyl peptidase IV | Pept | 0.77 | 2.2E−02 | 0.33 | 1.9E−01 | −0.04 | 9.4E−01 |
| | PUL 59 | | | | | | | | |
| Bova172_2995 | Predicted | hypothetical protein | Pept | 1.00 | 7.6E−05 | −0.85 | 3.6E−06 | −0.89 | 1.1E−02 |
| | PUL 59 | | | | | | | | |
| Bova172_2996 | Predicted | hypothetical protein | | 1.09 | 1.4E−05 | −1.98 | 2.9E−24 | −0.69 | 3.2E−02 |
| | PUL 59 | | | | | | | | |
| Bova172_2997 | Predicted | hypothetical protein | | 0.40 | 3.7E−01 | −2.25 | 6.5E−10 | 0.04 | 9.5E−01 |
| | PUL 59 | | | | | | | | |
| Bova172_2998 | Predicted | Cell surface glycan-binding | SusD | 0.17 | 5.5E−01 | −1.92 | 4.1E−19 | −0.58 | 1.1E−01 |
| | PUL 59 | lipoprotein, SusD family | | | | | | | |
| Bova172_{—}2999 | Predicted | Outer membrane TonB-dependent | Trans | 1.13 | 4.1E−24 | −3.80 | 8.3E−241 | −0.25 | 1.5E−01 |
| | PUL 59 | transporter, SusC family | | | | | | | |
| Bova172_{—}3000 | Predicted | Putative anti-sigma factor | REG | 2.19 | 1.8E−107 | −6.51 | 0.0E+00 | −0.08 | 6.6E−01 |
| | PUL 59 | | | | | | | | |
| Bova172_3001 | Predicted | RNA polymerase sigma-70 factor | REG | 0.61 | 5.9E−02 | 1.32 | 3.7E−08 | 1.76 | 2.6E−03 |
| | PUL 59 | | | | | | | | |
| Bova172_3037 | Predicted | hypothetical protein | GH18 | 0.19 | 5.1E−01 | −0.72 | 7.7E−04 | 0.76 | 2.4E−02 |
| | PUL 60 | | | | | | | | |
| Bova172_3038 | Predicted | hypothetical protein | | −2.40 | 2.1E−05 | −0.03 | 9.4E−01 | −0.67 | 1.0E+00 |
| | PUL 60 | | | | | | | | |
| Bova172_3039 | Predicted | Cell surface glycan-binding | SusD | −1.43 | 1.1E−04 | −0.34 | 2.4E−01 | −0.79 | 9.7E−02 |
| | PUL 60 | lipoprotein, SusD family | | | | | | | |
| Bova172_3040 | Predicted | Outer membrane TonB-dependent | Trans | −0.80 | 2.3E−04 | −1.20 | 1.2E−15 | −0.38 | 2.0E−01 |
| | PUL 60 | transporter, SusC family | | | | | | | |
| Bova172_3041 | Predicted | Putative anti-sigma factor | REG | −0.31 | 2.1E−02 | −2.46 | 9.1E−115 | −0.02 | 9.4E−01 |
| | PUL 60 | | | | | | | | |
| Bova172_3042 | Predicted | RNA polymerase ECF-type sigma | REG | −0.34 | 1.1E−01 | 0.06 | 7.7E−01 | 1.04 | 4.4E−04 |
| | PUL 60 | factor | | | | | | | |
| Bova172_3055 | Predicted | Outer membrane TonB-dependent | Trans | 2.31 | 1.8E−19 | −0.40 | 1.4E−02 | 1.60 | 3.5E−07 |
| | PUL 61 | transporter, SusC family | | | | | | | |
| Bova172_3056 | Predicted | Cell surface glycan-binding | SusD | 0.52 | 2.9E−01 | 1.38 | 5.0E−04 | 0.21 | 6.8E−01 |
| | PUL 61 | lipoprotein, SusD family | | | | | | | |
| Bova172_3057 | Predicted | Two-component system sensor | | 1.84 | 3.2E−03 | 2.03 | 7.7E−08 | 0.34 | 5.0E−01 |
| | PUL 61 | histidine kinase | | | | | | | |
| Bova172_3103 | Predicted | FIG01423360: glycoside hydrolase | GH130 | 0.49 | 1.5E−02 | 0.94 | 2.6E−05 | 0.24 | 5.6E−01 |
| | PUL 62 | | | | | | | | |
| Bova172_3104 | Predicted | hypothetical protein | Trans | −0.55 | 3.5E−02 | 0.15 | 6.1E−01 | −0.49 | 2.8E−01 |
| | PUL 62 | | | | | | | | |
| Bova172_3105 | Predicted | hypothetical protein | | −1.27 | 1.1E−04 | 0.19 | 5.4E−01 | −1.01 | 3.4E−02 |
| | PUL 62 | | | | | | | | |
| Bova172_3106 | Predicted | Outer membrane TonB-dependent | Trans | −0.09 | 7.5E−01 | 0.21 | 3.0E−01 | 0.15 | 7.5E−01 |
| | PUL 62 | transporter, SusC family | | | | | | | |
| Bova172_3107 | Predicted | Cell surface glycan-binding | SusD | 0.31 | 3.8E−01 | 0.97 | 2.4E−03 | −0.10 | 8.5E−01 |
| | PUL 62 | lipoprotein, SusD family | | | | | | | |
| Bova172_3108 | Predicted | hypothetical protein | | 0.44 | 3.7E−01 | 0.97 | 4.6E−02 | −0.08 | 1.0E+00 |
| | PUL 62 | | | | | | | | |
| Bova172_3109 | Predicted | BaeS-type histidine kinase/ | REG | 0.64 | 3.9E−06 | 0.94 | 5.1E−19 | 0.71 | 4.9E−04 |
| | PUL 62 | OmpR-type DNA-binding response | | | | | | | |
| | | regulator | | | | | | | |
| Bova172_3119 | Predicted | hypothetical protein | | −0.25 | 5.8E−01 | 0.75 | 2.5E−02 | −0.47 | 3.3E−01 |
| | PUL 63 | | | | | | | | |
| Bova172_3120 | Predicted | UDP-glucose 4-epimerase (EC | | −0.62 | 2.8E−01 | 0.11 | 8.4E−01 | 0.13 | 1.0E+00 |
| | PUL 63 | 5.1.3.2) | | | | | | | |
| Bova172_3121 | Predicted | Glucuronyl hydrolase | GH88 | 0.72 | 1.4E−01 | 0.21 | 6.2E−01 | 0.22 | 1.0E+00 |
| | PUL 63 | | | | | | | | |
| Bova172_3122 | Predicted | hypothetical protein | PL35 | 0.29 | 5.2E−01 | 1.19 | 2.4E−03 | −0.36 | 4.7E−01 |
| | PUL 63 | | | | | | | | |
| Bova172_3123 | Predicted | hypothetical protein | PL35 | −0.75 | 2.7E−02 | 0.26 | 4.2E−01 | 0.21 | 6.8E−01 |
| | PUL 63 | | | | | | | | |
| Bova172_3124 | Predicted | Cell surface glycan-binding | SusD | −0.31 | 4.5E−01 | 0.40 | 2.8E−01 | −0.14 | 7.9E−01 |
| | PUL 63 | lipoprotein, SusD family | | | | | | | |
| Bova172_3125 | Predicted | Outer membrane TonB-dependent | Trans | −0.49 | 7.2E−02 | −0.17 | 4.6E−01 | 0.32 | 4.5E−01 |
| | PUL 63 | transporter, SusC family | | | | | | | |
| Bova172_3126 | Predicted | sialic acid-specific 9-O- | | −0.68 | 5.8E−06 | 0.87 | 1.2E−11 | 0.28 | 4.7E−01 |
| | PUL 63 | acetylesterase | | | | | | | |
| Bova172_3127 | Predicted | BaeS-type histidine kinase/ | REG | 0.49 | 4.6E−02 | 2.63 | 1.5E−41 | −0.04 | 9.4E−01 |
| | PUL 63 | OmpR-type DNA-binding response | | | | | | | |
| | | regulator | | | | | | | |
| Bova172_3128 | Predicted | Radical SAM protein BT_3168 | | 0.38 | 2.6E−04 | −0.50 | 3.6E−08 | 1.69 | 1.2E−20 |
| | PUL 63 | | | | | | | | |
| Bova172_3129 | Predicted | hypothetical protein | | −1.06 | 1.5E−04 | 0.68 | 8.6E−03 | −2.12 | 2.2E−06 |
| | PUL 63 | | | | | | | | |
| Bova172_3130 | Predicted | hypothetical protein | | −2.13 | 1.2E−17 | −0.38 | 7.2E−02 | −2.48 | 2.6E−11 |
| | PUL 63 | | | | | | | | |
| Bova172_3131 | Predicted | hypothetical protein | | −1.56 | 5.0E−15 | −1.43 | 2.7E−20 | −3.01 | 8.1E−20 |
| | PUL 63 | | | | | | | | |
| Bova172_3132 | Predicted | hypothetical protein | | −0.44 | 1.5E−01 | −1.71 | 3.8E−13 | −1.18 | 7.6E−04 |
| | PUL 63 | | | | | | | | |
| Bova172_3133 | Predicted | Cell surface glycan-binding | SusD | −1.52 | 3.6E−12 | −1.79 | 3.3E−24 | −2.48 | 2.8E−18 |
| | PUL 63 | lipoprotein, SusD family | | | | | | | |
| Bova172_3134 | Predicted | Outer membrane TonB-dependent | Trans | −0.70 | 1.0E−11 | −2.38 | 2.4E−160 | −2.06 | 9.1E−49 |
| | PUL 63 | transporter, SusC family | | | | | | | |
| Bova172_3135 | Predicted | hypothetical protein | REG | −0.47 | 1.8E−04 | −1.17 | 3.5E−25 | −0.62 | 2.1E−02 |
| | PUL 63 | | | | | | | | |
| Bova172_3136 | Predicted | hypothetical protein | REG | −0.88 | 7.4E−11 | −0.03 | 8.3E−01 | 0.79 | 1.5E−03 |
| | PUL 63 | | | | | | | | |
| Bova172_3139 | Predicted | hypothetical protein | | 0.22 | 3.7E−01 | −2.17 | 8.9E−22 | −0.26 | 4.9E−01 |
| | PUL 64 | | | | | | | | |
| Bova172_3140 | Predicted | Alpha-1,2-mannosidase | GH92 | −0.69 | 1.4E−04 | −0.88 | 6.9E−05 | −0.15 | 7.0E−01 |
| | PUL 64 | | | | | | | | |
| Bova172_3141 | Predicted | Outer membrane TonB-dependent | Trans | −0.51 | 7.8E−04 | −0.29 | 9.7E−02 | −1.43 | 5.6E−06 |
| | PUL 64 | transporter, SusC family | | | | | | | |
| Bova172_3142 | Predicted | Cell surface glycan-binding | SusD | 0.63 | 3.2E−03 | 0.28 | 2.9E−01 | −0.72 | 1.0E−01 |
| | PUL 64 | lipoprotein, SusD family | | | | | | | |
| Bova172_3143 | Predicted | hypothetical protein | | 0.85 | 5.7E−05 | −0.13 | 6.0E−01 | −0.56 | 1.9E−01 |
| | PUL 64 | | | | | | | | |
| Bova172_3144 | Predicted | alpha-L-rhamnosidase (EC | GH78 | −0.42 | 2.3E−01 | 0.73 | 3.5E−02 | −0.28 | 5.6E−01 |
| | PUL 64 | 3.2.1.40) | | | | | | | |
| Bova172_3145 | Predicted | beta-galactosidase (EC 3.2.1.23) | GH2 | 1.08 | 1.0E−07 | 1.08 | 1.5E−07 | −0.06 | 9.2E−01 |
| | PUL 64 | | | | | | | | |
| Bova172_3146 | Predicted | hypothetical protein | | 0.30 | 1.0E−01 | 1.40 | 9.1E−11 | −0.29 | 4.5E−01 |
| | PUL 64 | | | | | | | | |
| Bova172_3147 | Predicted | Rhamnogalacturonides | GH105 | −1.34 | 1.2E−18 | −0.22 | 2.5E−01 | −0.44 | 2.0E−01 |
| | PUL 64 | degradation protein RhiN | | | | | | | |
| Bova172_3148 | Predicted | hypothetical protein | | −1.31 | 5.0E−02 | −0.15 | 7.9E−01 | −0.08 | 1.0E+00 |
| | PUL 64 | | | | | | | | |
| Bova172_3149 | Predicted | hypothetical protein | | −0.46 | 1.6E−06 | −0.05 | 6.4E−01 | 0.43 | 6.2E−03 |
| | PUL 64 | | | | | | | | |
| Bova172_3150 | Predicted | alpha-galactosidase (EC 3.2.1.22) | GH27- | −0.57 | 4.4E−03 | 0.06 | 7.8E−01 | −0.28 | 4.1E−01 |
| | PUL 64 | | CBM51 | | | | | | |
| Bova172_3161 | Predicted | BaeS-type histidine kinase/ | REG | 0.18 | 3.3E−01 | 0.97 | 3.3E−14 | −0.21 | 4.6E−01 |
| | PUL 65 | OmpR-type DNA-binding response | | | | | | | |
| | | regulator | | | | | | | |
| Bova172_3162 | Predicted | hypothetical protein | | −0.30 | 6.0E−01 | 0.43 | 4.3E−01 | 0.03 | 1.0E+00 |
| | PUL 65 | | | | | | | | |
| Bova172_3163 | Predicted | hypothetical protein | | −0.69 | 1.1E−01 | 0.49 | 2.0E−01 | 0.81 | 1.4E−01 |
| | PUL 65 | | | | | | | | |
| Bova172_3164 | Predicted | Outer membrane TonB-dependent | Trans | 0.16 | 6.2E−01 | 0.97 | 4.9E−08 | 0.05 | 9.3E−01 |
| | PUL 65 | transporter, SusC family | | | | | | | |
| Bova172_3165 | Predicted | hypothetical protein | SusD | 0.09 | 8.0E−01 | 0.97 | 3.5E−05 | 0.93 | 9.6E−02 |
| | PUL 65 | | | | | | | | |
| Bova172_3166 | Predicted | hypothetical protein | | 0.17 | 7.2E−01 | 1.80 | 4.1E−10 | −0.07 | 8.9E−01 |
| | PUL 65 | | | | | | | | |
| Bova172_3167 | Predicted | Putative lipase | | 0.84 | 3.5E−09 | −1.00 | 2.7E−17 | 0.95 | 1.3E−04 |
| | PUL 65 | | | | | | | | |
| Bova172_3168 | Predicted | hypothetical protein | | −0.69 | 2.4E−01 | 0.12 | 8.3E−01 | 0.02 | 1.0E+00 |
| | PUL 65 | | | | | | | | |
| Bova172_3169 | Predicted | hypothetical protein | GH26 | −0.10 | 8.6E−01 | 2.64 | 1.1E−05 | 0.02 | 1.0E+00 |
| | PUL 65 | | | | | | | | |
| Bova172_3170 | Predicted | Mannan endo-1,4-beta- | GH26 | 0.52 | 3.7E−01 | 2.74 | 2.6E−07 | −0.08 | 1.0E+00 |
| | PUL 65 | mannosidase | | | | | | | |
| Bova172_3171 | Predicted | 4-O-beta-D-mannosyl-D-glucose | GH130 | 0.09 | 8.7E−01 | 2.80 | 2.9E−08 | 0.02 | 9.6E−01 |
| | PUL 65 | phosphorylase (EC 2.4.1.281) | | | | | | | |
| Bova172_3172 | Predicted | Glucuronide transporter UidB | Trans | 0.23 | 6.9E−01 | 3.64 | 2.7E−08 | 0.03 | 9.6E−01 |
| | PUL 65 | | | | | | | | |
| Bova172_3173 | Predicted | hypothetical protein | CE7 | −0.20 | 7.4E−01 | 3.95 | 4.1E−08 | −0.52 | 3.2E−01 |
| | PUL 65 | | | | | | | | |
| Bova172_3174 | Predicted | Cellobiose epimerase (EC | EPI | 0.01 | 9.9E−01 | 3.92 | 1.6E−12 | 0.44 | 3.8E−01 |
| | PUL 65 | 5.1.3.11) | | | | | | | |
| Bova172_3175 | Predicted | hypothetical protein | CE | −0.05 | 9.2E−01 | 3.08 | 4.3E−09 | −0.34 | 4.9E−01 |
| | PUL 65 | | | | | | | | |
| Bova172_3176 | Predicted | Alkaline phosphatase (EC 3.1.3.1) | | 0.42 | 3.1E−01 | 2.60 | 5.8E−22 | −1.00 | 8.5E−02 |
| | PUL 65 | | | | | | | | |
| Bova172_3177 | Predicted | hypothetical protein | | −0.70 | 7.4E−02 | 2.07 | 2.9E−11 | −0.05 | 9.4E−01 |
| | PUL 65 | | | | | | | | |
| Bova172_3178 | Predicted | Alpha-1,2-mannosidase | GH92 | −0.23 | 2.5E−01 | 1.73 | 1.5E−29 | 0.67 | 6.1E−02 |
| | PUL 65 | | | | | | | | |
| Bova172_3184 | Predicted | hypothetical protein | | −0.04 | 9.5E−01 | 1.41 | 3.0E−03 | 0.81 | 1.0E+00 |
| | PUL 66 | | | | | | | | |
| Bova172_3185 | Predicted | hypothetical protein | Pept | −0.60 | 2.4E−01 | 0.85 | 1.1E−01 | −0.95 | 1.0E+00 |
| | PUL 66 | | | | | | | | |
| Bova172_3186 | Predicted | hypothetical protein | SusD | 0.44 | 4.4E−01 | 0.73 | 9.6E−02 | −0.23 | 6.4E−01 |
| | PUL 66 | | | | | | | | |
| Bova172_3187 | Predicted | Outer membrane TonB-dependent | Trans | −0.71 | 1.3E−02 | 1.25 | 7.8E−05 | −0.24 | 5.7E−01 |
| | PUL 66 | transporter, SusC family | | | | | | | |
| Bova172_3188 | Predicted | hypothetical protein | | −0.49 | 2.0E−02 | −0.77 | 2.3E−04 | −0.99 | 3.8E−05 |
| | PUL 66 | | | | | | | | |
| Bova172_{—}3192 | Predicted | arylsulfatase A precursor | Sulf | 1.21 | 5.2E−03 | −0.61 | 4.5E−02 | 0.20 | 7.0E−01 |
| | PUL 67 | | | | | | | | |
| Bova172_3193 | Predicted | hypothetical protein | | 0.71 | 5.7E−02 | −0.94 | 1.2E−03 | −0.02 | 9.7E−01 |
| | PUL 67 | | | | | | | | |
| Bova172_3194 | Predicted | hypothetical protein | | −0.23 | 6.0E−01 | −0.92 | 1.0E−02 | −0.28 | 5.6E−01 |
| | PUL 67 | | | | | | | | |
| Bova172_3195 | Predicted | hypothetical protein | | 0.37 | 2.3E−01 | −0.27 | 2.0E−01 | 0.46 | 3.1E−01 |
| | PUL 67 | | | | | | | | |
| Bova172_{—}3196 | Predicted | putative beta-xylosidase | GH43_31 | 1.01 | 9.3E−03 | 0.33 | 2.3E−01 | −0.04 | 9.5E−01 |
| | PUL 67 | | | | | | | | |
| Bova172_3197 | Predicted | Outer membrane TonB-dependent | Trans | 0.65 | 4.1E−02 | 0.66 | 5.8E−03 | 0.60 | 1.9E−01 |
| | PUL 67 | transporter, SusC family | | | | | | | |
| Bova172_3198 | Predicted | Cell surface glycan-binding | SusD | 0.38 | 4.0E−01 | 0.82 | 1.5E−02 | 0.29 | 5.7E−01 |
| | PUL 67 | lipoprotein, SusD family | | | | | | | |
| Bova172_3199 | Predicted | Outer membrane TonB-dependent | Trans | 0.52 | 2.3E−01 | 1.11 | 3.6E−04 | −0.89 | 1.2E−01 |
| | PUL 67 | transporter, SusC family | | | | | | | |
| Bova172_3200 | Predicted | Cell surface glycan-binding | SusD | 0.54 | 2.0E−01 | 0.88 | 2.5E−03 | −0.05 | 9.4E−01 |
| | PUL 67 | lipoprotein, SusD family | | | | | | | |
| Bova172_3201 | Predicted | hypothetical protein | | 0.41 | 2.0E−01 | 0.15 | 5.5E−01 | 0.16 | 7.5E−01 |
| | PUL 67 | | | | | | | | |
| Bova172_3202 | Predicted | BaeS-type histidine kinase/ | REG | −1.66 | 1.3E−27 | −0.34 | 1.4E−02 | −0.99 | 1.5E−04 |
| | PUL 67 | OmpR-type DNA-binding response | | | | | | | |
| | | regulator | | | | | | | |
| Bova172_3203 | Predicted | alpha-L-arabinofuranosidase (EC | GH51 | −1.63 | 6.2E−06 | 0.06 | 8.6E−01 | 0.20 | 6.9E−01 |
| | PUL 67 | 3.2.1.55) | | | | | | | |
| Bova172_3204 | Predicted | Putative sulfatase | Sulf | −0.82 | 7.5E−02 | 0.00 | 1.0E+00 | −0.46 | 3.5E−01 |
| | PUL 67 | | | | | | | | |
| Bova172_3205 | Predicted | putative secreted xylosidase | GH43 | −0.49 | 2.9E−01 | 0.28 | 5.2E−01 | 0.21 | 1.0E+00 |
| | PUL 67 | | | | | | | | |
| Bova172_3206 | Predicted | Arylsulfatase (EC 3.1.6.1) | Sulf | −0.93 | 3.5E−02 | 0.45 | 2.7E−01 | −0.37 | 1.0E+00 |
| | PUL 67 | | | | | | | | |
| Bova172_3207 | Predicted | beta-galactosidase (EC 3.2.1.23) | GH2 | 0.02 | 9.7E−01 | 1.20 | 8.1E−04 | 0.21 | 6.9E−01 |
| | PUL 67 | | | | | | | | |
| Bova172_3209 | Predicted | Regulatory protein SusR | REG | −1.22 | 3.1E−26 | −0.20 | 6.7E−02 | −0.34 | 1.5E−01 |
| | PUL 68 | | | | | | | | |
| Bova172_3210 | Predicted | Outer membrane TonB-dependent | Trans | −1.65 | 2.6E−14 | −0.85 | 3.3E−05 | −0.49 | 1.6E−01 |
| | PUL 68 | transporter, SusC family | | | | | | | |
| Bova172_3211 | Predicted | Cell surface glycan-binding | SusD | −0.75 | 2.6E−02 | −0.51 | 1.1E−01 | 0.26 | 6.0E−01 |
| | PUL 68 | lipoprotein, SusD family | | | | | | | |
| Bova172_3212 | Predicted | hypothetical protein | | −1.22 | 2.1E−04 | −0.50 | 8.5E−02 | −0.89 | 8.4E−02 |
| | PUL 68 | | | | | | | | |
| Bova172_3213 | Predicted | dextranase precursor | GH66 | −1.21 | 3.4E−04 | −0.17 | 6.2E−01 | −0.57 | 2.3E−01 |
| | PUL 68 | | | | | | | | |
| Bova172_3214 | Predicted | alpha-xylosidase (EC 3.2.1.177) | GH31 | −1.19 | 2.3E−04 | 0.12 | 7.0E−01 | −0.24 | 6.2E−01 |
| | PUL 68 | | | | | | | | |
| Bova172_3216 | Predicted | hypothetical protein | GH16 | 0.38 | 2.6E−01 | −0.06 | 8.5E−01 | 0.20 | 7.1E−01 |
| | PUL 69 | | | | | | | | |
| Bova172_3217 | Predicted | Cell surface glycan-binding | SusD | −0.13 | 7.7E−01 | 0.50 | 1.7E−01 | 0.30 | 5.5E−01 |
| | PUL 69 | lipoprotein, SusD family | | | | | | | |
| Bova172_3218 | Predicted | Outer membrane TonB-dependent | Trans | −0.33 | 4.2E−01 | −0.01 | 9.8E−01 | −0.04 | 9.5E−01 |
| | PUL 69 | transporter, SusC family | | | | | | | |
| Bova172_3219 | Predicted | Cell surface glycan-binding | SusD | −0.82 | 4.3E−02 | 0.48 | 2.1E−01 | −0.64 | 2.4E−01 |
| | PUL 69 | lipoprotein, SusD family | | | | | | | |
| Bova172_3220 | Predicted | Outer membrane TonB-dependent | Trans | −0.72 | 1.5E−02 | −0.51 | 5.0E−02 | 0.18 | 7.2E−01 |
| | PUL 69 | transporter, SusC family | | | | | | | |
| Bova172_3221 | Predicted | BaeS-type histidine kinase/ | REG | 0.15 | 1.8E−01 | −0.48 | 6.7E−08 | 0.44 | 5.6E−03 |
| | PUL 69 | OmpR-type DNA-binding response | | | | | | | |
| | | regulator | | | | | | | |
| Bova172_3222 | Predicted | alpha-L-fucosidase (EC 3.2.1.51) | GH29 | 0.71 | 1.6E−01 | −0.54 | 1.3E−01 | 0.66 | 2.0E−01 |
| | PUL 69 | | | | | | | | |
| Bova172_3223 | Predicted | Neuraminidase NanP | GH123 | 0.52 | 3.4E−01 | −0.42 | 2.8E−01 | 0.60 | 1.0E+00 |
| | PUL 69 | | | | | | | | |
| Bova172_3224 | Predicted | Uncharacterized protein BF0855 | GH95 | 0.33 | 5.4E−01 | 0.58 | 1.7E−01 | 0.03 | 1.0E+00 |
| | PUL 69 | | | | | | | | |
| Bova172_3225 | Predicted | alpha-L-fucosidase (EC 3.2.1.51) | GH29-PL30 | 0.87 | 4.8E−02 | 0.93 | 3.0E−03 | −0.96 | 4.6E−02 |
| | PUL 69 | | | | | | | | |
| Bova172_3226 | Predicted | beta-glucosidase (EC 3.2.1.21) | GH3 | 0.70 | 1.1E−01 | 0.56 | 9.1E−02 | 0.11 | 8.4E−01 |
| | PUL 69 | | | | | | | | |
| Bova172_3229 | Predicted | GntR family transcriptional | REG | 0.37 | 3.1E−02 | 0.21 | 1.1E−01 | 0.37 | 2.2E−01 |
| | PUL 70 | regulator | | | | | | | |
| Bova172_3230 | Predicted | Outer membrane TonB-dependent | Trans | −0.20 | 3.7E−02 | −4.47 | 8.6E−128 | 0.07 | 6.4E−01 |
| | PUL 70 | transporter, SusC family | | | | | | | |
| Bova172_3231 | Predicted | Cell surface glycan-binding | SusD | −1.76 | 7.8E−34 | −4.34 | 2.7E−42 | −0.73 | 3.5E−03 |
| | PUL 70 | lipoprotein, SusD family | | | | | | | |
| Bova172_3232 | Predicted | hypothetical protein | | −2.03 | 1.9E−43 | −2.52 | 1.3E−47 | −0.92 | 1.1E−03 |
| | PUL 70 | | | | | | | | |
| Bova172_3233 | Predicted | Alginate lyase precursor (EC | PL6_1 | −1.89 | 1.4E−42 | −2.29 | 8.7E−44 | −0.86 | 1.5E−03 |
| | PUL 70 | 4.2.2.3) | | | | | | | |
| Bova172_3234 | Predicted | hypothetical protein | PL17 2 | −2.02 | 1.3E−14 | −1.21 | 7.7E−06 | −1.25 | 1.2E−02 |
| | PUL 70 | | | | | | | | |
| Bova172_3235 | Predicted | putative pectin degradation protein | | −2.14 | 9.1E−12 | −1.39 | 3.2E−06 | −0.98 | 7.9E−02 |
| | PUL 70 | | | | | | | | |
| Bova172_3236 | Predicted | Predicted mannuronate transporter | Trans | −1.91 | 1.2E−16 | −0.97 | 7.9E−06 | −0.82 | 5.7E−02 |
| | PUL 70 | | | | | | | | |
| Bova172_3237 | Predicted | Acetoin(diacetyl) reductase | | −1.60 | 9.9E−14 | −0.96 | 7.9E−06 | −0.86 | 4.1E−02 |
| | PUL 70 | | | | | | | | |
| Bova172_3238 | Predicted | hypothetical protein | PL38 | −2.31 | 5.9E−13 | −0.80 | 2.0E−02 | −0.34 | 4.9E−01 |
| | PUL 70 | | | | | | | | |
| Bova172_3239 | Predicted | putative esterase | | −1.42 | 1.2E−03 | −0.09 | 8.3E−01 | −0.24 | 6.3E−01 |
| | PUL 70 | | | | | | | | |
| Bova172_3240 | Predicted | hypothetical protein | | −1.49 | 6.8E−07 | 0.03 | 9.2E−01 | −1.33 | 2.4E−02 |
| | PUL 70 | | | | | | | | |
| Bova172_3241 | Predicted | alpha-xylosidase (EC 3.2.1.177) | GH31 | 0.43 | 4.5E−01 | −0.88 | 1.0E−01 | 0.03 | 1.0E+00 |
| | PUL 70 | | | | | | | | |
| Bova172_3242 | Predicted | alpha-xylosidase (EC 3.2.1.177) | | −0.74 | 1.9E−01 | −1.83 | 8.3E−03 | 0.25 | 1.0E+00 |
| | PUL 70 | | | | | | | | |
| Bova172_3243 | Predicted | Putative lipase | | −0.63 | 3.5E−04 | 0.19 | 2.1E−01 | 0.12 | 7.7E−01 |
| | PUL 70 | | | | | | | | |
| Bova172_3244 | Predicted | Cysteine synthase (EC 2.5.1.47) | | 1.20 | 1.1E−29 | −1.43 | 1.5E−73 | 2.78 | 1.1E−101 |
| | PUL 70 | | | | | | | | |
| Bova172_{—}3245 | Predicted | β-glucosidase | GH3 | 1.81 | 7.1E−12 | −1.91 | 1.3E−28 | 0.50 | 1.7E−01 |
| | PUL 70 | | | | | | | | |
| Bova172_3246 | Predicted | hypothetical protein | | 0.50 | 2.4E−01 | −0.56 | 9.1E−02 | 0.03 | 9.5E−01 |
| | PUL 70 | | | | | | | | |
| Bova172_3247 | Predicted | Cell surface glycan-binding | SusD | −0.12 | 7.7E−01 | −0.04 | 8.9E−01 | 0.03 | 9.5E−01 |
| | PUL 70 | lipoprotein, SusD family | | | | | | | |
| Bova172_3248 | Predicted | Outer membrane TonB-dependent | Trans | 0.31 | 2.1E−01 | −0.61 | 3.4E−03 | −0.01 | 9.8E−01 |
| | PUL 70 | transporter, SusC family | | | | | | | |
| Bova172_3249 | Predicted | β-(1-3)-endoglucanase | GH16 | −0.73 | 2.4E−02 | −1.45 | 1.4E−05 | 0.11 | 8.4E−01 |
| | PUL 70 | | | | | | | | |
| Bova172_3250 | Predicted | hypothetical protein | REG | 0.20 | 2.2E−02 | −0.72 | 1.8E−21 | −0.16 | 2.3E−01 |
| | PUL 70 | | | | | | | | |
| Bova172_3251 | Predicted | hypothetical protein | | 0.20 | 3.6E−01 | −0.57 | 6.1E−05 | −1.03 | 1.2E−02 |
| | PUL 70 | | | | | | | | |
| Bova172_3252 | Predicted | Outer membrane TonB-dependent | Trans | 0.04 | 9.2E−01 | −2.69 | 3.5E−25 | 0.03 | 9.6E−01 |
| | PUL 70 | transporter, SusC family | | | | | | | |
| Bova172_3253 | Predicted | Cell surface glycan-binding | SusD | 0.51 | 3.0E−01 | −2.05 | 1.8E−06 | 0.13 | 1.0E+00 |
| | PUL 70 | lipoprotein, SusD family | | | | | | | |
| Bova172_3254 | Predicted | Arabinan endo-1,5-alpha-L- | GH43_28 | 0.46 | 4.1E−01 | −2.19 | 9.5E−08 | −0.21 | 1.0E+00 |
| | PUL 70 | arabinosidase A | | | | | | | |
| Bova172_3255 | Predicted | hypothetical protein | GH144 | 0.60 | 2.9E−01 | −0.55 | 2.1E−01 | −0.18 | 1.0E+00 |
| | PUL 70 | | | | | | | | |
| Bova172_3256 | Predicted | beta-glucosidase (EC 3.2.1.21) | GH3 | −0.08 | 8.7E−01 | 0.20 | 6.4E−01 | −0.51 | 1.0E+00 |
| | PUL 70 | | | | | | | | |
| Bova172_3257 | Predicted | beta-glucosidase (EC 3.2.1.21) | GH3 | −0.20 | 2.3E−01 | 2.17 | 1.8E−50 | 0.30 | 3.1E−01 |
| | PUL 71 | | | | | | | | |
| Bova172_3258 | Predicted | Pyridoxamine 5′-phosphate | | 0.27 | 2.3E−01 | 2.24 | 1.1E−39 | −0.29 | 4.8E−01 |
| | PUL 71 | oxidase-related, FMN-binding | | | | | | | |
| Bova172_3259 | Predicted | 3′-to-5′ exoribonuclease RNase R | | −0.22 | 8.9E−03 | 1.74 | 1.1E−107 | 0.68 | 6.1E−08 |
| | PUL 71 | | | | | | | | |
| Bova172_3260 | Predicted | hypothetical protein | | 0.12 | 7.8E−01 | 0.37 | 2.1E−01 | −0.22 | 6.4E−01 |
| | PUL 71 | | | | | | | | |
| Bova172_3261 | Predicted | hypothetical protein | | −0.41 | 3.8E−01 | −0.32 | 4.0E−01 | 0.04 | 9.5E−01 |
| | PUL 71 | | | | | | | | |
| Bova172_3262 | Predicted | hypothetical protein | | −0.22 | 6.1E−01 | −0.45 | 2.2E−01 | 0.32 | 4.8E−01 |
| | PUL 71 | | | | | | | | |
| Bova172_3263 | Predicted | FIG00939679: hypothetical protein | | −0.31 | 5.3E−01 | −0.18 | 6.3E−01 | −0.54 | 2.7E−01 |
| | PUL 71 | | | | | | | | |
| Bova172_3264 | Predicted | hypothetical protein | Pept | −0.16 | 7.8E−01 | −0.41 | 3.3E−01 | −0.46 | 1.0E+00 |
| | PUL 71 | | | | | | | | |
| Bova172_3265 | Predicted | Cell surface glycan-binding | SusD | −0.17 | 7.0E−01 | −0.97 | 5.3E−04 | −1.01 | 3.5E−02 |
| | PUL 71 | lipoprotein, SusD family | | | | | | | |
| Bova172_3266 | Predicted | Outer membrane TonB-dependent | Trans | 0.90 | 1.9E−04 | −2.01 | 1.6E−40 | −0.70 | 2.1E−02 |
| | PUL 71 | transporter, SusC family | | | | | | | |
| Bova172_3267 | Predicted | Putative anti-sigma factor | REG | 0.21 | 1.6E−01 | −2.52 | 1.2E−94 | −0.80 | 2.7E−04 |
| | PUL 71 | | | | | | | | |
| Bova172_3268 | Predicted | hypothetical protein | REG | −0.86 | 1.4E−08 | −0.19 | 1.8E−01 | 0.75 | 4.6E−03 |
| | PUL 71 | | | | | | | | |
| Bova172_3291 | Predicted | Chitinase (EC 3.2.1.14) | GH18 | 0.24 | 3.3E−01 | 0.36 | 6.1E−02 | 0.26 | 4.7E−01 |
| | PUL 72 | | | | | | | | |
| Bova172_3292 | Predicted | BaeS-type histidine kinase/ | REG | 0.31 | 6.5E−02 | −0.15 | 3.2E−01 | 0.06 | 8.4E−01 |
| | PUL 72 | OmpR-type DNA-binding response | | | | | | | |
| | | regulator | | | | | | | |
| Bova172_3293 | Predicted | hypothetical protein | | −1.04 | 1.1E−01 | −0.89 | 1.6E−01 | 0.03 | 1.0E+00 |
| | PUL 72 | | | | | | | | |
| Bova172_3294 | Predicted | Outer membrane TonB-dependent | Trans | 1.05 | 8.0E−03 | 0.98 | 3.5E−03 | −0.31 | 4.9E−01 |
| | PUL 72 | transporter, SusC family | | | | | | | |
| Bova172_3295 | Predicted | Cell surface glycan-binding | SusD | 0.77 | 8.5E−02 | 0.47 | 2.0E−01 | −0.29 | 5.5E−01 |
| | PUL 72 | lipoprotein, SusD family | | | | | | | |
| Bova172_3296 | Predicted | hypothetical protein | | 0.40 | 4.5E−01 | 0.56 | 2.4E−01 | 0.21 | 1.0E+00 |
| | PUL 72 | | | | | | | | |
| Bova172_3297 | Predicted | Hypothetical glycoside hydrolase, | GH43_34 | 0.36 | 5.3E−01 | −0.03 | 9.4E−01 | −0.19 | 1.0E+00 |
| | PUL 72 | family 43, similar to arabinosidase | | | | | | | |
| Bova172_3298 | Predicted | putative xylanase | GH30_4 | 0.52 | 3.1E−01 | 0.27 | 5.3E−01 | 0.03 | 9.5E−01 |
| | PUL 72 | | | | | | | | |
| Bova172_3299 | Predicted | hypothetical protein | REG | 0.50 | 1.7E−04 | 0.80 | 7.0E−12 | 0.37 | 7.0E−02 |
| | PUL 72 | | | | | | | | |
| Bova172_3300 | Predicted | Outer membrane TonB-dependent | Trans | 0.15 | 5.2E−01 | 0.21 | 1.9E−01 | −1.34 | 1.2E−05 |
| | PUL 72 | transporter, SusC family | | | | | | | |
| Bova172_3301 | Predicted | hypothetical protein | SusD | −0.09 | 7.8E−01 | 0.77 | 4.1E−03 | −0.11 | 8.3E−01 |
| | PUL 72 | | | | | | | | |
| Bova172_3302 | Predicted | hypothetical protein | | 0.47 | 2.9E−01 | 1.85 | 4.9E−05 | −0.38 | 4.3E−01 |
| | PUL 72 | | | | | | | | |
| Bova172_3303 | Predicted | hypothetical protein | GH105 | 0.45 | 2.4E−01 | 1.35 | 1.9E−05 | −0.71 | 1.4E−01 |
| | PUL 72 | | | | | | | | |
| Bova172_3304 | Predicted | hypothetical protein | | 0.14 | 6.7E−01 | 2.63 | 4.7E−18 | −0.41 | 3.6E−01 |
| | PUL 72 | | | | | | | | |
| Bova172_3305 | Predicted | hypothetical protein | | 0.47 | 3.5E−01 | 2.01 | 1.5E−05 | −0.36 | 4.7E−01 |
| | PUL 72 | | | | | | | | |
| Bova172_3306 | Predicted | hypothetical protein | | 0.31 | 5.2E−01 | 2.22 | 3.2E−08 | −0.21 | 6.8E−01 |
| | PUL 72 | | | | | | | | |
| Bova172_3307 | Predicted | hypothetical protein | | 0.12 | 7.8E−01 | 1.62 | 3.1E−07 | −0.07 | 8.9E−01 |
| | PUL 72 | | | | | | | | |
| Bova172_3314 | Predicted | beta-glucosidase (EC 3.2.1.21) | GH3 | −0.13 | 6.7E−01 | −0.05 | 8.7E−01 | 1.05 | 2.5E−04 |
| | PUL 73 | | | | | | | | |
| Bova172_3315 | Predicted | Xylan 1,4-beta-xylosidase (EC | GH43_12 | −0.08 | 8.7E−01 | 1.68 | 8.7E−04 | 0.40 | 4.1E−01 |
| | PUL 73 | 3.2.1.37) | | | | | | | |
| Bova172_3316 | Predicted | Xylan 1,4-beta-xylosidase (EC | GH43_12 | −0.36 | 5.2E−01 | 4.47 | 1.8E−10 | 0.43 | 3.9E−01 |
| | PUL 73 | 3.2.1.37) | | | | | | | |
| Bova172_3317 | Predicted | Endo-1,4-beta-xylanase (EC | GH5_4 | 0.35 | 5.2E−01 | 3.60 | 6.3E−09 | 0.17 | 7.5E−01 |
| | PUL 73 | 3.2.1.8) | | | | | | | |
| Bova172_3318 | Predicted | Outer membrane TonB-dependent | Trans | 0.11 | 8.4E−01 | 3.88 | 2.6E−12 | −0.03 | 9.6E−01 |
| | PUL 73 | transporter, SusC family | | | | | | | |
| Bova172_3319 | Predicted | Cell surface glycan-binding | SusD | 0.42 | 4.6E−01 | 3.91 | 2.2E−09 | 0.08 | 8.8E−01 |
| | PUL 73 | lipoprotein, SusD family | | | | | | | |
| Bova172_3320 | Predicted | hypothetical protein | | 0.48 | 3.8E−01 | 2.53 | 6.0E−05 | 0.12 | 8.1E−01 |
| | PUL 73 | | | | | | | | |
| Bova172_3321 | Predicted | Xyloglucan-specific endo-beta-1,4- | GH9 | 0.28 | 6.2E−01 | 3.09 | 1.0E−05 | 0.14 | 7.9E−01 |
| | PUL 73 | glucanase (EC 3.2.1.151) | | | | | | | |
| Bova172_3322 | Predicted | BaeS-type histidine kinase/ | REG | −0.04 | 8.2E−01 | 1.44 | 5.6E−19 | −0.26 | 4.5E−01 |
| | PUL 73 | OmpR-type DNA-binding response | | | | | | | |
| | | regulator | | | | | | | |
| Bova172_3323 | Predicted | alpha-xylosidase (EC 3.2.1.177) | GH31 | 0.11 | 8.4E−01 | 3.55 | 2.7E−09 | −0.02 | 9.7E−01 |
| | PUL 73 | | | | | | | | |
| Bova172_3324 | Predicted | beta-galactosidase (EC 3.2.1.23) | GH2 | 0.72 | 1.8E−01 | 4.70 | 4.1E−19 | 0.33 | 5.0E−01 |
| | PUL 73 | | | | | | | | |
| Bova172_3325 | Predicted | beta-glucosidase (EC 3.2.1.21) | GH3 | 0.75 | 1.8E−01 | 2.79 | 4.4E−06 | 0.58 | 2.6E−01 |
| | PUL 73 | | | | | | | | |
| Bova172_3331 | Predicted | Sialic acid-specific 9-O- | | −0.54 | 1.4E−02 | 2.22 | 8.4E−38 | −0.19 | 7.0E−01 |
| | PUL 74 | acetylesterase | | | | | | | |
| Bova172_3332 | Predicted | hypothetical protein | | −0.56 | 4.9E−02 | 0.94 | 1.9E−06 | 0.17 | 7.4E−01 |
| | PUL 74 | | | | | | | | |
| Bova172_3333 | Predicted | alpha-rhamnosidase | GH78 | −0.06 | 8.9E−01 | 2.83 | 4.4E−20 | −0.48 | 3.3E−01 |
| | PUL 74 | | | | | | | | |
| Bova172_3334 | Predicted | Sodium: galactoside symporter | Trans | −0.41 | 3.2E−01 | 2.71 | 5.3E−14 | 0.13 | 8.2E−01 |
| | PUL 74 | family protein | | | | | | | |
| Bova172_3335 | Predicted | 4-O-beta-D-mannosyl-D-glucose | GH130 | −0.05 | 9.1E−01 | 2.57 | 9.3E−13 | −0.06 | 9.2E−01 |
| | PUL 74 | phosphorylase (EC 2.4.1.281) | | | | | | | |
| Bova172_3336 | Predicted | hypothetical protein | GH140 | −0.52 | 2.3E−01 | 2.54 | 3.6E−11 | −0.12 | 8.3E−01 |
| | PUL 74 | | | | | | | | |
| Bova172_3337 | Predicted | hypothetical protein | GH5_5 | 0.55 | 2.9E−01 | 2.72 | 3.6E−11 | −0.24 | 6.3E−01 |
| | PUL 74 | | | | | | | | |
| Bova172_3338 | Predicted | beta-galactosidase (EC 3.2.1.23) | GH2 | 0.75 | 8.1E−03 | 0.73 | 7.7E−04 | 0.46 | 2.6E−01 |
| | PUL 74 | | | | | | | | |
| Bova172_3339 | Predicted | hypothetical protein | GH5_5 | 0.04 | 8.7E−01 | 0.18 | 3.6E−01 | 1.01 | 9.6E−05 |
| | PUL 74 | | | | | | | | |
| Bova172_3340 | Predicted | hypothetical protein | | −0.18 | 6.3E−01 | 1.55 | 2.5E−08 | −0.44 | 3.8E−01 |
| | PUL 74 | | | | | | | | |
| Bova172_3341 | Predicted | Cell surface glycan-binding | SusD | 0.06 | 8.6E−01 | 0.92 | 2.4E−05 | −0.43 | 3.4E−01 |
| | PUL 74 | lipoprotein, SusD family | | | | | | | |
| Bova172_3342 | Predicted | Outer membrane TonB-dependent | Trans | −0.29 | 2.2E−01 | 1.06 | 1.1E−08 | −0.34 | 3.5E−01 |
| | PUL 74 | transporter, SusC family | | | | | | | |
| Bova172_3343 | Predicted | hypothetical protein | GH5_2 | −0.46 | 8.2E−02 | 0.20 | 3.5E−01 | 0.14 | 7.6E−01 |
| | PUL 74 | | | | | | | | |
| Bova172_3394 | Predicted | Outer membrane TonB-dependent | Trans | 0.83 | 6.1E−04 | −0.74 | 2.6E−04 | 0.11 | 7.8E−01 |
| | PUL 75 | transporter, SusC family | | | | | | | |
| Bova172_3395 | Predicted | Cell surface glycan-binding | SusD | 0.92 | 4.2E−03 | −1.30 | 3.8E−07 | 0.29 | 5.1E−01 |
| | PUL 75 | lipoprotein, SusD family | | | | | | | |
| Bova172_3396 | Predicted | hypothetical protein | GH115 | 0.08 | 8.5E−01 | 0.41 | 2.0E−01 | −0.11 | 8.4E−01 |
| | PUL 75 | | | | | | | | |
| Bova172_3397 | Predicted | hypothetical protein | GH | 0.60 | 2.1E−01 | 0.06 | 8.7E−01 | 0.66 | 2.0E−01 |
| | PUL 75 | | | | | | | | |
| Bova172_3398 | Predicted | hypothetical protein | GH105 | −0.12 | 8.2E−01 | 0.35 | 3.8E−01 | −0.16 | 1.0E+00 |
| | PUL 75 | | | | | | | | |
| Bova172_3399 | Predicted | Platelet-activating factor | | 2.77 | 5.3E−41 | −2.63 | 1.5E−45 | 2.31 | 2.2E−19 |
| | PUL 75 | acetylhydrolase IB gamma subunit | | | | | | | |
| | | (EC 3.1.1.47) | | | | | | | |
| Bova172_3400 | Predicted | Arabinan endo-1,5-alpha-L- | GH43_3 | 1.73 | 1.3E−07 | −1.25 | 6.9E−06 | 1.48 | 6.0E−04 |
| | PUL 75 | arabinosidase (EC 3.2.1.99) | | | | | | | |
| Bova172_3401 | Predicted | hypothetical protein | | 1.12 | 3.3E−03 | −0.73 | 6.7E−03 | −0.79 | 9.7E−02 |
| | PUL 75 | | | | | | | | |
| Bova172_3402 | Predicted | Outer membrane TonB-dependent | Trans | 0.49 | 5.7E−02 | −1.18 | 7.3E−10 | 0.06 | 9.1E−01 |
| | PUL 75 | transporter, SusC family | | | | | | | |
| Bova172_3403 | Predicted | Cell surface glycan-binding | SusD | 1.52 | 9.8E−16 | −1.94 | 2.6E−43 | 1.46 | 1.0E−08 |
| | PUL 75 | lipoprotein, SusD family | | | | | | | |
| Bova172_3404 | Predicted | hypothetical protein | | 1.39 | 9.7E−04 | −1.21 | 7.4E−07 | −0.34 | 4.9E−01 |
| | PUL 75 | | | | | | | | |
| Bova172_3405 | Predicted | hypothetical protein | | −0.48 | 4.6E−02 | −1.20 | 6.7E−09 | 0.50 | 2.4E−01 |
| | PUL 75 | | | | | | | | |
| Bova172_3406 | Predicted | hypothetical protein | | −0.10 | 8.1E−01 | 0.30 | 3.3E−01 | −0.02 | 9.6E−01 |
| | PUL 75 | | | | | | | | |
| Bova172_3407 | Predicted | Beta-mannosidase (EC 3.2.1.25) | GH2 | 0.20 | 5.3E−01 | 1.30 | 1.1E−06 | −0.15 | 7.3E−01 |
| | PUL 75 | | | | | | | | |
| Bova172_3408 | Predicted | FIG01423360: glycoside hydrolase | GH130 | 0.06 | 9.1E−01 | 1.11 | 9.7E−03 | 0.29 | 5.7E−01 |
| | PUL 75 | | | | | | | | |
| Bova172_3409 | Predicted | Beta-mannosidase (EC 3.2.1.25) | GH2 | 0.92 | 3.6E−02 | 1.47 | 3.7E−06 | 0.11 | 8.4E−01 |
| | PUL 75 | | | | | | | | |
| Bova172_3410 | Predicted | hypothetical protein | GH105 | −0.04 | 9.4E−01 | 1.15 | 7.4E−03 | 0.03 | 1.0E+00 |
| | PUL 75 | | | | | | | | |
| Bova172_3424 | Predicted | RNA polymerase ECF-type sigma | REG | 0.37 | 5.3E−02 | −0.58 | 1.3E−04 | −0.31 | 4.6E−01 |
| | PUL 76 | factor | | | | | | | |
| Bova172_3425 | Predicted | Putative anti-sigma factor | REG | 0.53 | 8.8E−07 | −2.19 | 1.8E−206 | −0.90 | 2.0E−15 |
| | PUL 76 | | | | | | | | |
| Bova172_3426 | Predicted | Outer membrane TonB-dependent | Trans | −0.74 | 6.5E−07 | −0.72 | 1.2E−11 | −2.74 | 9.3E−84 |
| | PUL 76 | transporter, SusC family | | | | | | | |
| Bova172_3427 | Predicted | Cell surface glycan-binding | SusD | −0.60 | 6.2E−03 | 0.72 | 7.7E−05 | −3.01 | 1.1E−38 |
| | PUL 76 | lipoprotein, SusD family | | | | | | | |
| Bova172_3428 | Predicted | hypothetical protein | | −0.10 | 7.6E−01 | 1.25 | 1.6E−09 | −3.06 | 1.4E−28 |
| | PUL 76 | | | | | | | | |
| Bova172_3429 | Predicted | hypothetical protein | | −0.42 | 2.1E−01 | 1.43 | 1.8E−07 | −2.90 | 4.2E−14 |
| | PUL 76 | | | | | | | | |
| Bova172_3430 | Predicted | hypothetical protein | | −0.48 | 2.1E−01 | 2.09 | 6.3E−10 | −3.66 | 5.0E−13 |
| | PUL 76 | | | | | | | | |
| Bova172_3431 | Predicted | beta-glucosidase (EC 3.2.1.21) | GH3 | −0.59 | 6.8E−02 | 1.72 | 5.2E−10 | −2.00 | 5.3E−07 |
| | PUL 76 | | | | | | | | |
| Bova172_3432 | Predicted | beta-glucosidase (EC 3.2.1.21) | GH3 | −0.11 | 8.2E−01 | 2.47 | 2.1E−11 | −2.59 | 2.1E−06 |
| | PUL 76 | | | | | | | | |
| Bova172_3433 | Predicted | Rhamnogalacturonides | GH105 | −0.58 | 2.4E−01 | 1.96 | 1.4E−04 | −0.04 | 9.3E−01 |
| | PUL 76 | degradation protein RhiN | | | | | | | |
| Bova172_3434 | Predicted | hypothetical protein | | −0.01 | 9.8E−01 | 3.28 | 2.4E−10 | −1.76 | 2.8E−04 |
| | PUL 76 | | | | | | | | |
| Bova172_3435 | Predicted | alpha-galactosidase (EC 3.2.1.22) | | −0.48 | 3.2E−01 | 2.73 | 1.7E−07 | −1.29 | 1.0E−02 |
| | PUL 76 | | | | | | | | |
| Bova172_3436 | Predicted | hypothetical protein | CE7 | 0.98 | 2.3E−02 | 1.07 | 5.1E−03 | 0.05 | 9.4E−01 |
| | PUL 76 | | | | | | | | |
| Bova172_3437 | Predicted | beta-galactosidase (EC 3.2.1.23) | GH2 | 0.46 | 3.8E−01 | 2.10 | 9.3E−06 | −0.37 | 4.7E−01 |
| | PUL 76 | | | | | | | | |
| Bova172_3438 | Predicted | hypothetical protein | | 2.12 | 7.0E−53 | −2.67 | 1.6E−95 | 1.82 | 2.6E−18 |
| | PUL 76 | | | | | | | | |
| Bova172_3461 | Predicted | Two-component system sensor | REG | 1.35 | 2.6E−04 | −0.17 | 5.5E−01 | 1.00 | 4.4E−03 |
| | PUL 77 | histidine kinase/response regulator | | | | | | | |
| | | hybrid | | | | | | | |
| Bova172_3462 | Predicted | Two-component system sensor | | 0.87 | 7.8E−10 | −0.59 | 7.8E−07 | 1.27 | 4.5E−13 |
| | PUL 77 | histidine kinase/response regulator | | | | | | | |
| | | hybrid | | | | | | | |
| Bova172_3463 | Predicted | Outer membrane TonB-dependent | Trans | 0.58 | 9.1E−02 | −0.35 | 2.3E−01 | 0.58 | 1.5E−01 |
| | PUL 77 | transporter, SusC family | | | | | | | |
| Bova172_3464 | Predicted | Cell surface glycan-binding | SusD | −0.13 | 8.1E−01 | −0.02 | 9.7E−01 | 1.02 | 1.0E+00 |
| | PUL 77 | lipoprotein, SusD family | | | | | | | |
| Bova172_3465 | Predicted | hypothetical protein | | 0.14 | 7.8E−01 | 0.05 | 8.9E−01 | 0.39 | 4.4E−01 |
| | PUL 77 | | | | | | | | |
| Bova172_3466 | Predicted | spore peptidoglycan hydrolase (N- | GH | 0.19 | 7.0E−01 | −0.06 | 8.9E−01 | 0.48 | 1.0E+00 |
| | PUL 77 | acetylglucosaminidase) | | | | | | | |
| Bova172_3468 | Predicted | hypothetical protein | | −0.68 | 1.5E−16 | −4.44 | 0.0E+00 | −2.57 | 1.5E−53 |
| | PUL 78 | | | | | | | | |
| Bova172_3469 | Predicted | Sugar: proton symporter | | −2.13 | 8.2E−127 | −4.39 | 6.5E−203 | −3.45 | 4.6E−34 |
| | PUL 78 | | | | | | | | |
| Bova172_3470 | Predicted | hypothetical protein | CE | −2.00 | 1.1E−162 | −5.43 | 1.1E−288 | −1.90 | 7.6E−35 |
| | PUL 78 | | | | | | | | |
| Bova172_3471 | Predicted | hypothetical protein | CE | −2.13 | 7.0E−137 | −6.21 | 0.0E+00 | −1.88 | 1.4E−54 |
| | PUL 78 | | | | | | | | |
| Bova172_3472 | Predicted | ADP-ribosylation/Crystallin J1 | | −3.35 | 0.0E+00 | −5.22 | 0.0E+00 | −2.73 | 3.0E−53 |
| | PUL 78 | | | | | | | | |
| Bova172_3473 | Predicted | hypothetical protein | | −4.03 | 0.0E+00 | −5.17 | 3.0E−289 | −2.75 | 7.5E−57 |
| | PUL 78 | | | | | | | | |
| Bova172_3474 | Predicted | hypothetical protein | | −3.44 | 0.0E+00 | −5.45 | 0.0E+00 | −2.21 | 9.6E−73 |
| | PUL 78 | | | | | | | | |
| Bova172_3475 | Predicted | Cell surface glycan-binding | SusD | −2.16 | 2.0E−230 | −6.70 | 0.0E+00 | −1.41 | 1.7E−56 |
| | PUL 78 | lipoprotein, SusD family | | | | | | | |
| Bova172_3476 | Predicted | Outer membrane TonB-dependent | Trans | −1.75 | 3.1E−130 | −6.98 | 0.0E+00 | −1.03 | 1.7E−41 |
| | PUL 78 | transporter, SusC family | | | | | | | |
| Bova172_3477 | Predicted | Ribokinase (EC 2.7.1.15) | | −0.50 | 8.6E−18 | −7.37 | 0.0E+00 | 0.12 | 7.9E−02 |
| | PUL 78 | | | | | | | | |
| Bova172_3481 | Predicted | Outer membrane TonB-dependent | Trans | −0.04 | 8.1E−01 | −1.75 | 9.5E−59 | 0.34 | 2.8E−01 |
| | PUL 79 | transporter, SusC family | | | | | | | |
| Bova172_3482 | Predicted | Cell surface glycan-binding | SusD | −0.42 | 1.5E−01 | −1.20 | 2.4E−09 | 0.29 | 5.4E−01 |
| | PUL 79 | lipoprotein, SusD family | | | | | | | |
| Bova172_3483 | Predicted | probable secreted protein | | −0.18 | 6.6E−01 | −1.45 | 2.8E−07 | −0.04 | 9.5E−01 |
| | PUL 79 | | | | | | | | |
| Bova172_3484 | Predicted | Alpha-N-acetylglucosaminidase | GH89 | 0.99 | 1.3E−06 | −0.55 | 9.8E−05 | 0.47 | 2.4E−01 |
| | PUL 79 | (EC 3.2.1.50) | | | | | | | |
| Bova172_3485 | Predicted | Glycerate kinase (EC 2.7.1.31) | | −0.21 | 6.2E−01 | 1.35 | 1.6E−04 | 0.24 | 6.4E−01 |
| | PUL 79 | | | | | | | | |
| Bova172_3486 | Predicted | D-glycerate transporter (predicted) | | −0.52 | 2.6E−01 | 1.29 | 1.1E−03 | −0.57 | 2.8E−01 |
| | PUL 79 | | | | | | | | |
| Bova172_3487 | Predicted | N-acetylglucosamine related | | −0.52 | 3.0E−01 | 1.06 | 1.4E−02 | 0.03 | 1.0E+00 |
| | PUL 79 | transporter, NagX | | | | | | | |
| Bova172_3488 | Predicted | Alpha-N-acetylglucosaminidase | GH89 | 0.11 | 8.1E−01 | 1.70 | 9.3E−09 | 0.12 | 8.3E−01 |
| | PUL 79 | (EC 3.2.1.50) | | | | | | | |
| Bova172_3489 | Predicted | RNA polymerase ECF-type sigma | REG | 0.62 | 3.1E−02 | −0.83 | 1.7E−03 | 0.86 | 1.1E−02 |
| | PUL 79 | factor | | | | | | | |
| Bova172_3490 | Predicted | Putative anti-sigma factor | REG | 2.08 | 1.7E−48 | −3.60 | 1.2E−16 | 0.77 | 1.0E−04 |
| | PUL 79 | | | | | | | | |
| Bova172_3494 | Predicted | Putative anti-sigma factor | REG | −0.63 | 1.1E−05 | −2.00 | 5.7E−37 | −0.53 | 9.5E−03 |
| | PUL 80 | | | | | | | | |
| Bova172_3495 | Predicted | Outer membrane TonB-dependent | Trans | −1.28 | 5.6E−15 | −3.03 | 4.3E−75 | −0.73 | 5.4E−03 |
| | PUL 80 | transporter, SusC family | | | | | | | |
| Bova172_3496 | Predicted | hypothetical protein | SusD | −1.49 | 1.7E−06 | −2.08 | 2.7E−18 | −1.40 | 5.2E−03 |
| | PUL 80 | | | | | | | | |
| Bova172_3497 | Predicted | putative cellulase family | GH5_13 | −1.75 | 9.7E−06 | −1.40 | 1.6E−05 | −1.05 | 5.5E−02 |
| | PUL 80 | | | | | | | | |
| Bova172_3498 | Predicted | hypothetical protein | GH | −2.53 | 1.7E−14 | −2.05 | 1.8E−16 | −0.89 | 7.0E−02 |
| | PUL 80 | | | | | | | | |
| Bova172_3499 | Predicted | hypothetical protein | | −1.66 | 1.5E−06 | −0.90 | 1.7E−03 | −1.17 | 2.0E−02 |
| | PUL 80 | | | | | | | | |
| Bova172_3500 | Predicted | Uncharacterized protein YphG, | | −0.91 | 4.2E−04 | −1.10 | 4.8E−09 | −0.45 | 2.9E−01 |
| | PUL 80 | TPR-domain containing | | | | | | | |
| Bova172_3501 | Predicted | RNA polymerase ECF-type sigma | REG | 0.45 | 3.2E−04 | −1.98 | 4.9E−74 | 1.11 | 2.9E−11 |
| | PUL 80 | factor | | | | | | | |
| Bova172_3502 | Predicted | hypothetical protein | EPI | 0.38 | 6.0E−02 | −0.80 | 5.9E−07 | 1.15 | 2.1E−04 |
| | PUL 80 | | | | | | | | |
| Bova172_3506 | Predicted | sodium-solute symporter, putative | | −0.50 | 8.8E−02 | −0.56 | 1.9E−02 | 0.18 | 6.8E−01 |
| | PUL 81 | | | | | | | | |
| Bova172_3507 | Predicted | hypothetical protein | | −0.70 | 9.5E−03 | 0.38 | 1.0E−01 | −0.85 | 8.2E−02 |
| | PUL 81 | | | | | | | | |
| Bova172_3508 | Predicted | Outer membrane TonB-dependent | Trans | −0.10 | 7.6E−01 | 0.96 | 4.7E−06 | −0.82 | 6.4E−02 |
| | PUL 81 | transporter, SusC family | | | | | | | |
| Bova172_3509 | Predicted | Cell surface glycan-binding | SusD | 0.06 | 8.8E−01 | 0.73 | 9.8E−03 | −0.17 | 7.5E−01 |
| | PUL 81 | lipoprotein, SusD family | | | | | | | |
| Bova172_3510 | Predicted | hypothetical protein | | −0.57 | 1.4E−01 | 1.19 | 7.6E−05 | −0.04 | 9.4E−01 |
| | PUL 81 | | | | | | | | |
| Bova172_3511 | Predicted | hypothetical protein | | −0.51 | 2.0E−01 | 1.22 | 5.9E−05 | −0.40 | 4.1E−01 |
| | PUL 81 | | | | | | | | |
| Bova172_3512 | Predicted | hypothetical protein | | 0.14 | 6.9E−01 | 1.24 | 8.3E−11 | −0.65 | 1.2E−01 |
| | PUL 81 | | | | | | | | |
| Bova172_3513 | Predicted | Sialic acid-specific 9-O- | | −0.03 | 9.3E−01 | 1.25 | 3.3E−05 | 0.40 | 3.8E−01 |
| | PUL 81 | acetylesterase | | | | | | | |
| Bova172_3514 | Predicted | beta-glucosidase (EC 3.2.1.21) | GH3 | −0.64 | 6.8E−02 | 1.73 | 6.2E−12 | −0.24 | 6.3E−01 |
| | PUL 81 | | | | | | | | |
| Bova172_3515 | Predicted | Alpha-1,2-mannosidase | GH92 | 0.11 | 8.1E−01 | 2.24 | 1.0E−13 | −0.30 | 5.4E−01 |
| | PUL 81 | | | | | | | | |
| Bova172_3516 | Predicted | Alpha-glucosidase (EC 3.2.1.20) | GH97 | 0.85 | 1.3E−02 | 2.55 | 2.1E−21 | 0.44 | 2.9E−01 |
| | PUL 81 | | | | | | | | |
| Bova172_3766 | Predicted | hypothetical protein | | 0.27 | 2.1E−01 | 0.73 | 3.0E−06 | −0.72 | 2.1E−02 |
| | PUL 82 | | | | | | | | |
| Bova172_3767 | Predicted | hypothetical protein | CBM35 | 0.93 | 1.1E−02 | 0.17 | 5.9E−01 | −0.58 | 2.3E−01 |
| | PUL 82 | | | | | | | | |
| Bova172_3768 | Predicted | hypothetical protein | GH27 | 0.02 | 9.4E−01 | −0.66 | 4.2E−04 | 0.17 | 5.8E−01 |
| | PUL 82 | | | | | | | | |
| Bova172_3769 | Predicted | hypothetical protein | | −0.51 | 1.1E−01 | −0.33 | 2.0E−01 | −0.88 | 2.7E−02 |
| | PUL 82 | | | | | | | | |
| Bova172_3770 | Predicted | Cell surface glycan-binding | SusD | −0.97 | 1.5E−03 | 0.41 | 1.3E−01 | −2.55 | 9.4E−08 |
| | PUL 82 | lipoprotein, SusD family | | | | | | | |
| Bova172_3771 | Predicted | Outer membrane TonB-dependent | Trans | −1.40 | 4.6E−20 | −1.04 | 8.8E−16 | −1.94 | 2.2E−14 |
| | PUL 82 | transporter, SusC family | | | | | | | |
| Bova172_3772 | Predicted | Putative anti-sigma factor | REG | −0.59 | 4.4E−03 | −2.12 | 4.2E−27 | −0.59 | 1.1E−01 |
| | PUL 82 | | | | | | | | |
| Bova172_3773 | Predicted | hypothetical protein | REG | −0.15 | 6.6E−01 | 0.18 | 5.1E−01 | 0.31 | 4.6E−01 |
| | PUL 82 | | | | | | | | |
| Bova172_3851 | Predicted | Putative anti-sigma factor | REG | 0.31 | 5.2E−03 | −1.95 | 2.8E−76 | 0.23 | 2.8E−01 |
| | PUL 83 | | | | | | | | |
| Bova172_3852 | Predicted | RNA polymerase ECF-type sigma | REG | −0.68 | 4.0E−04 | −1.25 | 7.0E−12 | 0.80 | 8.2E−03 |
| | PUL 83 | factor | | | | | | | |
| Bova172_3853 | Predicted | Outer membrane TonB-dependent | Trans | −0.71 | 9.7E−13 | −3.75 | 1.7E−226 | −0.11 | 4.7E−01 |
| | PUL 83 | transporter, SusC family | | | | | | | |
| Bova172_3854 | Predicted | Cell surface glycan-binding | SusD | −1.03 | 1.1E−09 | −3.25 | 3.1E−67 | −0.92 | 2.8E−04 |
| | PUL 83 | lipoprotein, SusD family | | | | | | | |
| Bova172_3855 | Predicted | hypothetical protein | GH43_17 | −1.27 | 1.5E−11 | −3.01 | 2.8E−55 | −1.14 | 1.0E−03 |
| | PUL 83 | | | | | | | | |
| Bova172_3856 | Predicted | hypothetical protein | | −1.36 | 6.7E−11 | −2.71 | 8.4E−42 | −1.10 | 2.4E−03 |
| | PUL 83 | | | | | | | | |
| Bova172_3857 | Predicted | putative cellulase family | GH5_13 | −0.76 | 2.3E−04 | −2.06 | 5.1E−29 | −0.83 | 2.5E−02 |
| | PUL 83 | | | | | | | | |
| Bova172_3858 | Predicted | beta-N-acetylglucosaminidase (EC | GH20 | −1.61 | 2.8E−20 | −1.44 | 1.5E−20 | −1.43 | 9.4E−05 |
| | PUL 83 | 3.2.1.52) | | | | | | | |
| Bova172_3859 | Predicted | RNA polymerase ECF-type sigma | REG | −1.53 | 8.2E−14 | −0.77 | 5.2E−05 | −0.60 | 1.3E−01 |
| | PUL 84 | factor | | | | | | | |
| Bova172_{—}3860 | Predicted | Putative anti-sigma factor | REG | 1.52 | 1.2E−59 | −2.92 | 1.4E−209 | −0.22 | 7.0E−02 |
| | PUL 84 | | | | | | | | |
| Bova172_3861 | Predicted | FIG00410220: hypothetical protein | | 2.66 | 3.3E−15 | −2.68 | 3.0E−15 | 0.76 | 3.2E−02 |
| | PUL 84 | | | | | | | | |
| Bova172_{—}3862 | Predicted | Outer membrane TonB-dependent | Trans | 1.45 | 7.6E−18 | −1.85 | 8.0E−52 | −0.96 | 2.4E−06 |
| | PUL 84 | transporter, SusC family | | | | | | | |
| Bova172_3863 | Predicted | Cell surface glycan-binding | SusD | −0.51 | 1.6E−01 | −0.42 | 1.4E−01 | −1.37 | 9.5E−04 |
| | PUL 84 | lipoprotein, SusD family | | | | | | | |
| Bova172_3864 | Predicted | hypothetical protein | GH43_18 | −0.86 | 3.6E−02 | −0.45 | 1.3E−01 | −0.83 | 8.8E−02 |
| | PUL 84 | | | | | | | | |
| Bova172_3865 | Predicted | hypothetical protein | | −0.32 | 3.3E−01 | −0.64 | 2.9E−03 | −1.03 | 1.5E−02 |
| | PUL 84 | | | | | | | | |
| Bova172_3866 | Predicted | beta-galactosidase (EC 3.2.1.23) | GH42 | −0.25 | 6.1E−01 | 1.08 | 5.6E−03 | −0.10 | 8.5E−01 |
| | PUL 84 | | | | | | | | |
| Bova172_3867 | Predicted | beta-glucosidase (EC 3.2.1.21) | GH3 | 0.34 | 5.3E−01 | 0.93 | 1.3E−02 | −0.31 | 5.2E−01 |
| | PUL 84 | | | | | | | | |
| Bova172_3868 | Predicted | putative cellulase family | GH5_13 | −0.95 | 8.8E−02 | 1.52 | 9.7E−03 | −0.14 | 1.0E+00 |
| | PUL 84 | | | | | | | | |
| Bova172_3869 | Predicted | Putative alpha-glucosidase | GH97 | −0.58 | 1.5E−04 | 1.17 | 7.7E−18 | −0.44 | 1.7E−01 |
| | PUL 84 | | | | | | | | |
| Bova172_3885 | Predicted | Gliding motility-associated ABC | Trans | −0.39 | 2.6E−01 | 1.10 | 2.1E−04 | −0.14 | 7.8E−01 |
| | PUL 85 | transporter permease protein GldF/ | | | | | | | |
| | | Gliding motility-associated ABC | | | | | | | |
| | | transporter substrate-binding | | | | | | | |
| | | protein GldG | | | | | | | |
| Bova172_3886 | Predicted | Gliding motility-associated ABC | Trans | 0.34 | 5.1E−01 | 2.25 | 2.6E−07 | −0.31 | 5.4E−01 |
| | PUL 85 | transporter permease protein GldF/ | | | | | | | |
| | | Gliding motility-associated ABC | | | | | | | |
| | | transporter substrate-binding | | | | | | | |
| | | protein GldG | | | | | | | |
| Bova172_3887 | Predicted | Gliding motility-associated ABC | Trans | −0.32 | 5.8E−01 | 1.48 | 6.3E−03 | 0.13 | 1.0E+00 |
| | PUL 85 | transporter ATP-binding protein | | | | | | | |
| | | GldA | | | | | | | |
| Bova172_3888 | Predicted | hypothetical protein | | −1.04 | 1.1E−01 | −0.90 | 1.6E−01 | 0.03 | 1.0E+00 |
| | PUL 85 | | | | | | | | |
| Bova172_3889 | Predicted | hypothetical protein | Trans | 0.62 | 1.3E−01 | 1.08 | 2.0E−03 | 1.19 | 1.3E−02 |
| | PUL 85 | | | | | | | | |
| Bova172_3890 | Predicted | TonB-dependent receptor | Trans | 0.19 | 5.2E−01 | 0.00 | 1.0E+00 | −0.38 | 4.3E−01 |
| | PUL 85 | | | | | | | | |
| Bova172_3891 | Predicted | hypothetical protein | | 1.38 | 1.6E−03 | −0.39 | 1.2E−01 | 0.25 | 5.8E−01 |
| | PUL 85 | | | | | | | | |
| Bova172_3892 | Predicted | hypothetical protein | | 0.69 | 1.8E−01 | 0.62 | 5.7E−02 | −0.10 | 8.5E−01 |
| | PUL 85 | | | | | | | | |
| Bova172_3893 | Predicted | hypothetical protein | | 0.37 | 4.5E−01 | −0.52 | 1.2E−01 | −0.14 | 7.9E−01 |
| | PUL 85 | | | | | | | | |
| Bova172_3894 | Predicted | hypothetical protein | | −0.41 | 4.3E−01 | −0.93 | 7.4E−03 | −0.18 | 1.0E+00 |
| | PUL 85 | | | | | | | | |
| Bova172_3895 | Predicted | hypothetical protein | SusD | 0.52 | 3.7E−01 | −0.66 | 3.9E−02 | −0.13 | 8.1E−01 |
| | PUL 85 | | | | | | | | |
| Bova172_3896 | Predicted | Outer membrane TonB-dependent | Trans | 0.21 | 5.7E−01 | −1.62 | 1.2E−19 | 0.17 | 7.3E−01 |
| | PUL 85 | transporter, SusC family | | | | | | | |
| Bova172_3897 | Predicted | putative anti-sigma factor | REG | 0.88 | 2.0E−04 | −4.22 | 1.8E−106 | 1.22 | 5.5E−05 |
| | PUL 85 | | | | | | | | |
| Bova172_3898 | Predicted | RNA polymerase ECF-type sigma | REG | 0.32 | 2.6E−01 | 0.18 | 4.2E−01 | −0.08 | 8.5E−01 |
| | PUL 85 | factor | | | | | | | |
| Bova172_4045 | Predicted | RNA polymerase ECF-type sigma | REG | 1.48 | 4.6E−37 | −2.94 | 7.5E−155 | 2.51 | 9.8E−82 |
| | PUL 86 | factor | | | | | | | |
| Bova172_4046 | Predicted | Putative anti-sigma factor | REG | 0.51 | 1.5E−12 | −2.99 | 0.0E+00 | 1.08 | 3.5E−29 |
| | PUL 86 | | | | | | | | |
| Bova172_4047 | Predicted | Outer membrane TonB-dependent | Trans | 1.28 | 5.1E−11 | −0.14 | 4.3E−01 | 0.69 | 6.8E−03 |
| | PUL 86 | transporter, SusC family | | | | | | | |
| Bova172_4048 | Predicted | Cell surface glycan-binding | SusD | 0.36 | 3.3E−01 | 0.65 | 3.5E−02 | −0.43 | 3.5E−01 |
| | PUL 86 | lipoprotein, SusD family | | | | | | | |
| Bova172_4049 | Predicted | hypothetical protein | | 0.30 | 5.1E−01 | 0.59 | 1.4E−01 | 0.88 | 8.2E−02 |
| | PUL 86 | | | | | | | | |
| Bova172_4050 | Predicted | hypothetical protein | | −0.95 | 1.5E−06 | −1.33 | 5.8E−15 | −0.72 | 3.5E−02 |
| | PUL 86 | | | | | | | | |
| Bova172_4051 | Predicted | Kojibiose phosphorylase (EC | GH65 | 0.42 | 3.8E−01 | 1.64 | 1.3E−04 | −0.03 | 9.6E−01 |
| | PUL 86 | 2.4.1.230) | | | | | | | |
| Bova172_4052 | Predicted | Sialic acid-specific 9-O- | | 0.21 | 6.4E−01 | 0.93 | 9.5E−03 | 0.62 | 2.2E−01 |
| | PUL 86 | acetylesterase | | | | | | | |
| Bova172_4060 | Predicted | Cell surface glycan-binding | SusD | 0.23 | 6.9E−01 | 2.21 | 4.7E−04 | 0.12 | 1.0E+00 |
| | PUL 87 | lipoprotein, SusD family | | | | | | | |
| Bova172_4061 | Predicted | Outer membrane TonB-dependent | Trans | 1.09 | 6.5E−02 | 1.95 | 1.3E−05 | 0.19 | 7.2E−01 |
| | PUL 87 | transporter, SusC family | | | | | | | |
| Bova172_4076 | Predicted | Cell surface glycan-binding | SusD | −1.49 | 1.1E−05 | −0.07 | 8.6E−01 | −0.40 | 4.1E−01 |
| | PUL 88 | lipoprotein, SusD family | | | | | | | |
| Bova172_4077 | Predicted | Outer membrane TonB-dependent | Trans | −2.76 | 2.6E−27 | −0.39 | 1.4E−01 | −0.42 | 2.9E−01 |
| | PUL 88 | transporter, SusC family | | | | | | | |
| Bova172_4078 | Predicted | hypothetical protein | SusD | 2.43 | 8.8E−32 | −0.72 | 1.4E−07 | −0.61 | 3.1E−02 |
| | PUL 88 | | | | | | | | |
| Bova172_{—}4079 | Predicted | Outer membrane TonB-dependent | Trans | 1.08 | 6.6E−28 | −1.47 | 3.4E−81 | 0.20 | 2.6E−01 |
| | PUL 88 | transporter, SusC family | | | | | | | |
| Bova172_4080 | Predicted | Integrase | Int | −0.55 | 2.8E−02 | −0.36 | 1.1E−01 | −0.71 | 1.4E−01 |
| | PUL 88 | | | | | | | | |
| Bova172_4081 | Predicted | hypothetical protein | | −0.84 | 1.7E−01 | −0.90 | 1.6E−01 | 0.03 | 1.0E+00 |
| | PUL 88 | | | | | | | | |
| Bova172_4082 | Predicted | Outer membrane TonB-dependent | Trans | −2.39 | 1.7E−58 | −0.17 | 1.4E−01 | −0.92 | 1.1E−03 |
| | PUL 88 | transporter, SusC family | | | | | | | |
| Bova172_4083 | Predicted | Cell surface glycan-binding | SusD | −1.18 | 5.9E−09 | 0.06 | 6.7E−01 | −0.60 | 1.1E−01 |
| | PUL 88 | lipoprotein, SusD family | | | | | | | |
| Bova172_4111 | Predicted | Uncharacterized protein YphG, | | 0.28 | 3.0E−01 | −1.65 | 4.4E−15 | −0.45 | 3.0E−01 |
| | PUL 89 | TPR-domain containing | CBM61- | | | | | | |
| Bova172_4112 | Predicted | hypothetical protein | GH53 | −0.43 | 1.2E−01 | −0.73 | 7.3E−04 | 0.09 | 8.4E−01 |
| | PUL 89 | | | | | | | | |
| Bova172_4113 | Predicted | putative lipoprotein | | −0.56 | 6.0E−02 | −0.43 | 1.0E−01 | −0.25 | 5.9E−01 |
| | PUL 89 | | | | | | | | |
| Bova172_4114 | Predicted | Outer membrane TonB-dependent | Trans | −0.96 | 1.4E−05 | 0.24 | 2.5E−01 | −0.69 | 9.3E−02 |
| | PUL 89 | transporter, SusC family | | | | | | | |
| Bova172_4115 | Predicted | Cell surface glycan-binding | SusD | −0.01 | 9.7E−01 | 0.15 | 6.2E−01 | 0.12 | 8.3E−01 |
| | PUL 89 | lipoprotein, SusD family | | | | | | | |
| Bova172_4116 | Predicted | hypothetical protein | | −0.28 | 5.7E−01 | 0.37 | 4.0E−01 | 0.03 | 1.0E+00 |
| | PUL 89 | | | | | | | | |
| Bova172_4117 | Predicted | beta-galactosidase (EC 3.2.1.23) | GH35 | −0.07 | 8.5E−01 | 0.96 | 1.7E−04 | 0.19 | 7.1E−01 |
| | PUL 89 | | | | | | | | |
| Bova172_4118 | Predicted | hypothetical protein | GH147 | −0.65 | 7.5E−02 | 1.03 | 5.0E−04 | −0.04 | 9.5E−01 |
| | PUL 89 | | | | | | | | |
| Bova172_4220 | Predicted | Predicted transcriptional regulator | REG | 1.74 | 1.5E−46 | −1.38 | 9.6E−27 | 1.93 | 5.7E−60 |
| | PUL 90 | of N-Acetylglucosamine utilization, | | | | | | | |
| | | GntR family | | | | | | | |
| Bova172_4221 | Predicted | mannose-6-phosphate isomerase, | | 0.22 | 3.9E−05 | −3.87 | 0.0E+00 | 1.16 | 2.3E−65 |
| | PUL 90 | class I | | | | | | | |
| Bova172_4222 | Predicted | ROK family sugar kinase or | ROK | −0.26 | 2.7E−02 | −2.11 | 2.1E−34 | −0.32 | 1.5E−01 |
| | PUL 90 | transcriptional regulator | | | | | | | |
| Bova172_4223 | Predicted | Outer membrane TonB-dependent | Trans | −1.03 | 5.7E−25 | −1.57 | 2.4E−40 | −1.02 | 3.1E−11 |
| | PUL 90 | transporter, SusC family | | | | | | | |
| Bova172_4224 | Predicted | Cell surface glycan-binding | SusD | −1.58 | 4.0E−23 | −1.63 | 8.0E−20 | −1.60 | 2.5E−07 |
| | PUL 90 | lipoprotein, SusD family | | | | | | | |
| Bova172_4225 | Predicted | hypothetical protein | | −1.14 | 2.2E−09 | −1.46 | 1.9E−12 | −1.62 | 4.0E−05 |
| | PUL 90 | | | | | | | | |
| Bova172_4226 | Predicted | hypothetical protein | GH76 | −1.69 | 1.2E−21 | −1.36 | 5.6E−13 | −1.66 | 6.8E−07 |
| | PUL 90 | | | | | | | | |
| Bova172_4227 | Predicted | putative alpha-1,6-mannanase | GH76 | −1.80 | 3.0E−20 | −1.10 | 4.0E−08 | −2.37 | 2.8E−08 |
| | PUL 90 | | | | | | | | |
| Bova172_4228 | Predicted | Alpha-1,2-mannosidase | GH92 | −1.31 | 9.3E−18 | −1.01 | 4.7E−13 | −1.28 | 1.4E−06 |
| | PUL 90 | | | | | | | | |
| Bova172_4229 | Predicted | hypothetical protein | Trans | −0.47 | 1.2E−01 | −0.10 | 7.1E−01 | −0.34 | 4.1E−01 |
| | PUL 90 | | | | | | | | |
| Bova172_4252 | Predicted | RNA polymerase ECF-type sigma | REG | −1.39 | 6.2E−15 | −0.76 | 3.0E−06 | 0.04 | 9.4E−01 |
| | PUL 91 | factor | | | | | | | |
| Bova172_4253 | Predicted | Putative anti-sigma factor | REG | −1.15 | 1.4E−22 | −1.46 | 4.5E−28 | −0.56 | 4.0E−03 |
| | PUL 91 | | | | | | | | |
| Bova172_4254 | Predicted | Outer membrane TonB-dependent | Trans | −1.52 | 4.8E−23 | −0.13 | 4.4E−01 | −1.56 | 2.5E−08 |
| | PUL 91 | transporter, SusC family | | | | | | | |
| Bova172_4255 | Predicted | Cell surface glycan-binding | SusD | −1.31 | 9.9E−08 | 0.30 | 2.4E−01 | −0.56 | 1.9E−01 |
| | PUL 91 | lipoprotein, SusD family | | | | | | | |
| Bova172_4256 | Predicted | hypothetical protein | | −1.56 | 1.2E−06 | −0.52 | 8.6E−02 | −0.68 | 1.4E−01 |
| | PUL 91 | | | | | | | | |
| Bova172_4257 | Predicted | putative patatin-like protein | | −1.51 | 2.5E−08 | −0.50 | 4.1E−02 | −0.56 | 2.0E−01 |
| | PUL 91 | | | | | | | | |
| Bova172_4258 | Predicted | hypothetical protein | GH18 | −1.63 | 1.4E−11 | −0.14 | 5.9E−01 | −0.58 | 1.9E−01 |
| | PUL 91 | | | | | | | | |
| Bova172_4338 | Predicted | Cell surface glycan-binding | SusD | 0.76 | 6.8E−02 | −0.20 | 4.7E−01 | 0.17 | 7.4E−01 |
| | PUL 92 | lipoprotein, SusD family | | | | | | | |
| Bova172_4339 | Predicted | Outer membrane TonB-dependent | Trans | 0.11 | 8.2E−01 | 1.16 | 2.0E−02 | −0.28 | 5.7E−01 |
| | PUL 92 | transporter, SusC family | | | | | | | |
| Bova172_4369 | Predicted | RNA polymerase ECF-type sigma | REG | −1.15 | 7.7E−09 | −2.01 | 7.5E−26 | −1.06 | 4.4E−03 |
| | PUL 93 | factor | | | | | | | |
| Bova172_4370 | Predicted | Putative anti-sigma factor | REG | 0.62 | 1.3E−09 | −2.65 | 2.6E−180 | −0.13 | 5.2E−01 |
| | PUL 93 | | | | | | | | |
| Bova172_4371 | Predicted | Outer membrane TonB-dependent | Trans | −0.49 | 1.0E−03 | −3.29 | 2.0E−106 | −0.94 | 1.8E−03 |
| | PUL 93 | transporter, SusC family | | | | | | | |
| Bova172_4372 | Predicted | Cell surface glycan-binding | SusD | −1.62 | 4.8E−13 | −2.27 | 3.3E−24 | −1.19 | 8.4E−03 |
| | PUL 93 | lipoprotein, SusD family | | | | | | | |
| Bova172_4373 | Predicted | hypothetical protein | | −2.30 | 1.3E−15 | −2.72 | 2.9E−25 | −0.67 | 1.8E−01 |
| | PUL 93 | | | | | | | | |
| Bova172_4374 | Predicted | beta-N-acetylglucosaminidase (EC | GH20 | −2.25 | 2.7E−25 | −2.79 | 9.6E−52 | −1.21 | 9.2E−03 |
| | PUL 93 | 3.2.1.52) | | | | | | | |
| Bova172_4375 | Predicted | beta-glycosyl hydrolase | GH20 | −1.55 | 2.9E−12 | −2.08 | 9.6E−26 | −1.81 | 5.2E−04 |
| | PUL 93 | | | | | | | | |
| Bova172_4376 | Predicted | N-acetylglucosamine related | | −2.57 | 1.5E−14 | −1.74 | 1.0E−12 | −0.10 | 8.5E−01 |
| | PUL 93 | transporter, NagX | | | | | | | |
| Bova172_4377 | Predicted | hypothetical protein | | 0.03 | 9.6E−01 | 1.27 | 4.7E−02 | 0.47 | 1.0E+00 |
| | PUL 94 | | | | | | | | |
| Bova172_4378 | Predicted | hypothetical protein | | −0.13 | 8.1E−01 | −0.42 | 4.4E−01 | 0.03 | 1.0E+00 |
| | PUL 94 | | | | | | | | |
| Bova172_4379 | Predicted | Cell surface glycan-binding | SusD | −0.31 | 5.8E−01 | 0.46 | 4.3E−01 | 0.13 | 1.0E+00 |
| | PUL 94 | lipoprotein, SusD family | | | | | | | |
| Bova172_4380 | Predicted | Outer membrane TonB-dependent | Trans | 2.07 | 4.5E−04 | 0.07 | 8.6E−01 | 0.88 | 8.6E−02 |
| | PUL 94 | transporter, SusC family | | | | | | | |
| Bova172_4396 | Predicted | Outer membrane TonB-dependent | Trans | −0.24 | 6.4E−01 | 0.41 | 3.4E−01 | 0.12 | 1.0E+00 |
| | PUL 95 | transporter, SusC family | | | | | | | |
| Bova172_4397 | Predicted | Cell surface glycan-binding | SusD | −0.14 | 8.1E−01 | 0.60 | 2.7E−01 | 0.03 | 1.0E+00 |
| | PUL 95 | lipoprotein, SusD family | | | | | | | |
| Bova172_4398 | Predicted | hypothetical protein | Pept | −1.06 | 1.1E−01 | 0.08 | 8.9E−01 | −0.08 | 1.0E+00 |
| | PUL 95 | | | | | | | | |
| Bova172_4399 | Predicted | hypothetical protein | | −0.48 | 4.0E−01 | −0.06 | 9.1E−01 | 0.13 | 1.0E+00 |
| | PUL 95 | | | | | | | | |
| Bova172_4400 | Predicted | hypothetical protein | | −0.06 | 9.2E−01 | 0.01 | 9.8E−01 | 0.03 | 1.0E+00 |
| | PUL 95 | | | | | | | | |
| Bova172_4580 | Predicted | Putative large secreted protein | GH95 | 0.72 | 5.3E−04 | −1.56 | 1.4E−20 | −0.12 | 7.4E−01 |
| | PUL 96 | SCO0341 | | | | | | | |
| Bova172_4581 | Predicted | xylosidase/arabinosidase | GH43_2 | 0.28 | 3.2E−01 | −2.68 | 2.6E−45 | 0.70 | 9.1E−02 |
| | PUL 96 | | | | | | | | |
| Bova172_4582 | Predicted | Endo-1,4-beta-xylanase (EC | GH43_2 | −1.38 | 2.1E−02 | −1.10 | 1.8E−02 | 0.41 | 1.0E+00 |
| | PUL 96 | 3.2.1.8) | | | | | | | |
| Bova172_4583 | Predicted | beta-glucosidase (EC 3.2.1.21) | GH3 | 0.56 | 9.8E−02 | −0.79 | 5.2E−04 | −0.71 | 1.4E−01 |
| | PUL 96 | | | | | | | | |
| Bova172_4584 | Predicted | hypothetical protein | | 0.96 | 8.3E−02 | −0.43 | 2.2E−01 | −0.47 | 3.4E−01 |
| | PUL 96 | | | | | | | | |
| Bova172_{—}4585 | Predicted | beta-glucosidase (EC 3.2.1.21) | GH3 | 1.16 | 3.4E−03 | −0.50 | 6.0E−02 | 0.00 | 9.9E−01 |
| | PUL 96 | | | | | | | | |
| Bova172_4586 | Predicted | hypothetical protein | GH2 | 0.25 | 6.6E−01 | 1.50 | 4.0E−03 | 0.23 | 1.0E+00 |
| | PUL 96 | | | | | | | | |
| Bova172_4587 | Predicted | hypothetical protein | | 0.32 | 5.2E−01 | 2.23 | 1.6E−08 | 0.25 | 6.2E−01 |
| | PUL 96 | | | | | | | | |
| Bova172_4588 | Predicted | hypothetical protein | | 0.76 | 5.2E−02 | 0.04 | 9.0E−01 | 0.46 | 3.0E−01 |
| | PUL 96 | | | | | | | | |
| Bova172_4589 | Predicted | hypothetical protein | | 0.05 | 9.4E−01 | 1.73 | 2.9E−03 | −0.19 | 1.0E+00 |
| | PUL 96 | | | | | | | | |
| Bova172_4590 | Predicted | Cell surface glycan-binding | SusD | −0.19 | 7.3E−01 | 1.80 | 5.4E−04 | −0.51 | 1.0E+00 |
| | PUL 96 | lipoprotein, SusD family | | | | | | | |
| Bova172_4591 | Predicted | Outer membrane TonB-dependent | Trans | 0.21 | 5.8E−01 | 1.70 | 4.3E−06 | 0.41 | 3.5E−01 |
| | PUL 96 | transporter, SusC family | | | | | | | |
| Bova172_4592 | Predicted | hypothetical protein | | −1.30 | 5.2E−02 | −0.87 | 1.3E−01 | 0.02 | 1.0E+00 |
| | PUL 96 | | | | | | | | |
| Bova172_4593 | Predicted | hypothetical protein | | −1.52 | 1.4E−02 | 0.35 | 3.8E−01 | 0.36 | 1.0E+00 |
| | PUL 96 | | | | | | | | |
| Bova172_4594 | Predicted | hypothetical protein | | −1.20 | 7.3E−02 | 0.09 | 8.8E−01 | 0.02 | 1.0E+00 |
| | PUL 96 | | | | | | | | |
| Bova172_4595 | Predicted | hypothetical protein | | −1.04 | 1.1E−01 | −0.73 | 2.3E−01 | 0.03 | 1.0E+00 |
| | PUL 96 | | | | | | | | |
| Bova172_4596 | Predicted | hypothetical protein | | −0.61 | 3.3E−10 | 0.01 | 9.5E−01 | 0.00 | 1.0E+00 |
| | PUL 96 | | | | | | | | |
| Bova172_4597 | Predicted | putative alpha-1,2-mannosidase | GH92 | 0.70 | 5.3E−02 | 2.06 | 1.0E−11 | −0.39 | 4.3E−01 |
| | PUL 96 | | | | | | | | |
| Bova172_4598 | Predicted | beta-galactosidase | GH2 | −0.61 | 2.7E−02 | 1.26 | 1.3E−08 | −0.22 | 6.2E−01 |
| | PUL 96 | | | | | | | | |
| Bova172_{—}4599 | Predicted | Endo-1,4-beta-xylanase (EC | GH43_2- | 1.21 | 1.2E−04 | −1.23 | 3.3E−15 | 0.43 | 3.0E−01 |
| | PUL 96 | 3.2.1.8) | CBM6- | | | | | | |
| | | | CBM6- | | | | | | |
| | | | CBM6 | | | | | | |
| Bova172_4600 | Predicted | Levanase (EC 3.2.1.65) | GH32 | −2.98 | 6.2E−299 | 1.30 | 2.3E−27 | 1.83 | 5.6E−37 |
| | PUL 96 | | | | | | | | |
| Bova172_4601 | Predicted | LacX protein, plasmid | EPI | 1.21 | 1.2E−34 | −0.23 | 1.1E−02 | 1.42 | 8.7E−47 |
| | PUL 96 | | | | | | | | |
| Bova172_4602 | Predicted | Prophage antirepressor | | 1.11 | 3.7E−25 | −1.71 | 3.3E−64 | 1.57 | 2.0E−54 |
| | PUL 96 | | | | | | | | |
| Bova172_4603 | Predicted | Outer membrane TonB-dependent | Trans | −4.38 | 0.0E+00 | 2.23 | 2.7E−66 | 0.38 | 6.0E−03 |
| | PUL 96 | transporter, SusC family | | | | | | | |
| Bova172_4604 | Predicted | Cell surface glycan-binding | SusD | −3.66 | 3.8E−81 | 3.18 | 2.1E−48 | −0.21 | 4.2E−01 |
| | PUL 96 | lipoprotein, SusD family | | | | | | | |
| Bova172_4605 | Predicted | hypothetical protein | | −2.93 | 4.2E−37 | 3.26 | 1.5E−41 | −0.29 | 3.2E−01 |
| | PUL 96 | | | | | | | | |
| Bova172_4606 | Predicted | Sucrose-6-phosphate hydrolase | GH32 | −0.36 | 1.3E−02 | 2.86 | 2.4E−59 | 1.95 | 2.4E−32 |
| | PUL 96 | (EC 3.2.1.26) | | | | | | | |
| Bova172_4607 | Predicted | Levanase (EC 3.2.1.65) | GH32 | −5.69 | 3.7E−291 | 4.05 | 5.0E−100 | 0.06 | 7.9E−01 |
| | PUL 96 | | | | | | | | |
| Bova172_4670 | Predicted | Outer membrane TonB-dependent | Trans | −0.32 | 2.8E−01 | 0.68 | 2.1E−02 | 1.30 | 1.2E−03 |
| | PUL 97 | transporter, SusC family | | | | | | | |
| Bova172_4671 | Predicted | Cell surface glycan-binding | SusD | −0.33 | 4.5E−01 | 1.69 | 7.0E−04 | −0.26 | 6.0E−01 |
| | PUL 97 | lipoprotein, SusD family | | | | | | | |
| Bova172_4702 | Predicted | Pullulanase (EC 3.2.1.41) | GH13_14 | −0.08 | 2.7E−01 | −1.08 | 5.0E−69 | −0.22 | 1.1E−01 |
| | PUL 98 | | | | | | | | |
| Bova172_4703 | Predicted | BaeS-type histidine kinase/ | REG | −0.35 | 4.8E−04 | −0.17 | 8.2E−02 | 1.03 | 8.5E−18 |
| | PUL 98 | OmpR-type DNA-binding response | | | | | | | |
| | | regulator | | | | | | | |
| Bova172_4704 | Predicted | Outer membrane TonB-dependent | Trans | −0.34 | 1.1E−01 | −0.05 | 7.8E−01 | −0.48 | 1.2E−01 |
| | PUL 98 | transporter, SusC family | | | | | | | |
| Bova172_4705 | Predicted | Cell surface glycan-binding | SusD | −0.35 | 2.1E−01 | −0.01 | 9.4E−01 | −1.59 | 4.5E−03 |
| | PUL 98 | lipoprotein, SusD family | | | | | | | |
| Bova172_4706 | Predicted | hypothetical protein | | −0.25 | 3.7E−01 | −0.13 | 4.8E−01 | 0.04 | 9.5E−01 |
| | PUL 98 | | | | | | | | |
| Bova172_4707 | Predicted | Endo-1,4-beta-xylanase (EC | GH10- | 0.26 | 3.9E−01 | −0.17 | 5.2E−01 | 0.47 | 2.5E−01 |
| | PUL 98 | 3.2.1.8) | CBM4- | | | | | | |
| | | | CBM4- | | | | | | |
| | | | GH10 | | | | | | |
| Bova172_4708 | Predicted | Sialic acid-specific 9-O- | | −0.14 | 7.1E−01 | 0.81 | 4.2E−03 | −0.62 | 2.1E−01 |
| | PUL 98 | acetylesterase | | | | | | | |
| Bova172_4709 | Predicted | Xyloside transporter XynT | Trans | −0.15 | 7.2E−01 | 1.06 | 1.0E−04 | 0.22 | 6.7E−01 |
| | PUL 98 | | | | | | | | |
| Bova172_4710 | Predicted | Endo-1,4-beta-xylanase (EC | GH10 | −0.45 | 2.6E−01 | 1.48 | 6.7E−07 | −0.85 | 1.4E−01 |
| | PUL 98 | 3.2.1.8) | | | | | | | |
| Bova172_4711 | Predicted | Xylosidase/arabinosidase | GH43_1 | −0.38 | 3.7E−01 | 1.76 | 4.5E−10 | −0.37 | 4.5E−01 |
| | PUL 98 | | | | | | | | |
| Bova172_4712 | Predicted | Xylan alpha-1,2-glucuronosidase | GH67 | 0.23 | 6.1E−01 | 2.20 | 5.1E−11 | 0.77 | 1.4E−01 |
| | PUL 98 | (EC 3.2.1.131) | | | | | | | |
| Bova172_4716 | Predicted | RNA polymerase sigma-70 factor | REG | 0.11 | 8.2E−01 | −0.65 | 8.8E−02 | −0.57 | 2.8E−01 |
| | PUL 99 | | | | | | | | |
| Bova172_4717 | Predicted | Putative anti-sigma factor | REG | −1.59 | 4.4E−13 | −1.66 | 1.1E−14 | −1.36 | 5.7E−04 |
| | PUL 99 | | | | | | | | |
| Bova172_4718 | Predicted | Outer membrane TonB-dependent | Trans | −1.45 | 1.4E−14 | 0.49 | 1.7E−02 | −1.67 | 3.5E−07 |
| | PUL 99 | transporter, SusC family | | | | | | | |
| Bova172_4719 | Predicted | Cell surface glycan-binding | SusD | −1.26 | 8.3E−05 | 1.25 | 2.0E−04 | −1.49 | 3.0E−03 |
| | PUL 99 | lipoprotein, SusD family | | | | | | | |
| Bova172_4720 | Predicted | hypothetical protein | | −0.80 | 3.1E−03 | 1.74 | 1.8E−12 | −0.52 | 1.8E−01 |
| | PUL 99 | | | | | | | | |
| Bova172_4731 | Predicted | Outer membrane TonB-dependent | Trans | 1.49 | 1.8E−16 | −1.16 | 2.2E−17 | 0.44 | 6.0E−02 |
| | PUL 100 | transporter, SusC family | | | | | | | |
| Bova172_4732 | Predicted | Cell surface glycan-binding | SusD | 0.99 | 2.7E−04 | −0.74 | 2.0E−05 | −0.03 | 9.5E−01 |
| | PUL 100 | lipoprotein, SusD family | | | | | | | |
| Bova172_4733 | Predicted | Mucin-desulfating sulfatase | Sulf | 0.57 | 1.2E−01 | −0.12 | 7.0E−01 | 0.17 | 7.4E−01 |
| | PUL 100 | | | | | | | | |
| Bova172_4734 | Predicted | beta-glycosyl hydrolase | GH20 | 0.40 | 2.9E−01 | 0.26 | 3.9E−01 | 0.32 | 4.8E−01 |
| | PUL 100 | | | | | | | | |
| Bova172_4735 | Predicted | beta-galactosidase (EC 3.2.1.23) | GH2 | −0.98 | 6.8E−10 | −2.04 | 3.4E−52 | −0.82 | 3.8E−03 |
| | PUL 100 | | | | | | | | |
| Bova172_4736 | Predicted | glycosyl hydrolase family 29 | GH29 | −1.18 | 3.8E−27 | −2.06 | 3.3E−103 | −0.58 | 1.5E−02 |
| | PUL 100 | (alpha-L-fucosidase) | | | | | | | |
| Bova172_4786 | Predicted | RNA polymerase ECF-type sigma | REG | −1.04 | 4.6E−25 | −2.51 | 6.3E−138 | 0.61 | 1.7E−04 |
| | PUL 101 | factor | | | | | | | |
| Bova172_{—}4787 | Predicted | Putative anti-sigma factor | REG | 1.02 | 1.0E−45 | −4.23 | 0.0E+00 | −0.89 | 1.1E−14 |
| | PUL 101 | | | | | | | | |
| Bova172_4788 | Predicted | Outer membrane TonB-dependent | Trans | −1.41 | 3.4E−44 | −2.17 | 1.8E−84 | −2.19 | 3.7E−36 |
| | PUL 101 | transporter, SusC family | | | | | | | |
| Bova172_4789 | Predicted | Cell surface glycan-binding | SusD | −2.02 | 5.2E−20 | −0.87 | 3.7E−05 | −2.80 | 3.1E−13 |
| | PUL 101 | lipoprotein, SusD family | | | | | | | |
| Bova172_4790 | Predicted | hypothetical protein | | −1.56 | 1.8E−06 | −0.30 | 3.9E−01 | −2.11 | 3.2E−05 |
| | PUL 101 | | | | | | | | |
| Bova172_4791 | Predicted | hypothetical protein | | −0.58 | 2.0E−02 | −0.25 | 3.1E−01 | −2.82 | 5.3E−09 |
| | PUL 101 | | | | | | | | |
| Bova172_4792 | Predicted | hypothetical protein | | −0.09 | 7.3E−01 | 0.10 | 6.9E−01 | −1.15 | 1.4E−03 |
| | PUL 101 | | | | | | | | |
| Bova172_4822 | Predicted | Cell surface glycan-binding | SusD | 1.45 | 4.6E−04 | 2.51 | 8.1E−29 | 0.17 | 7.3E−01 |
| | PUL 102 | lipoprotein, SusD family | | | | | | | |
| Bova172_4823 | Predicted | Outer membrane TonB-dependent | Trans | 0.56 | 4.7E−02 | 3.03 | 6.0E−57 | −0.82 | 6.1E−02 |
| | PUL 102 | transporter, SusC family | | | | | | | |
| Bova172_4824 | Predicted | FIG00938541: hypothetical protein | | −0.88 | 6.8E−07 | 2.31 | 1.0E−39 | −1.35 | 2.6E−03 |
| | PUL 102 | | | | | | | | |
| Bova172_4861 | Predicted | Outer membrane TonB-dependent | Trans | 1.22 | 1.3E−09 | −0.77 | 5.4E−07 | 1.29 | 5.0E−10 |
| | PUL 103 | transporter, SusC family | | | | | | | |
| Bova172_4862 | Predicted | hypothetical protein | SusD | 0.38 | 3.7E−01 | 0.41 | 3.2E−01 | 0.05 | 9.3E−01 |
| | PUL 103 | | | | | | | | |
| Bova172_4863 | Predicted | hypothetical protein | | −0.82 | 9.6E−02 | −0.30 | 5.8E−01 | −0.10 | 1.0E+00 |
| | PUL 103 | | | | | | | | |
| Bova172_4864 | Predicted | hypothetical protein | | −0.22 | 6.0E−01 | 0.20 | 6.0E−01 | −0.62 | 1.8E−01 |
| | PUL 103 | | | | | | | | |
| Bova172_4865 | Predicted | hypothetical protein | | 0.72 | 1.2E−01 | 1.63 | 6.8E−04 | −1.45 | 1.6E−02 |
| | PUL 103 | | | | | | | | |
| Bova172_4866 | Predicted | hypothetical protein | | 0.63 | 5.9E−05 | 0.29 | 1.6E−02 | 0.03 | 9.4E−01 |
| | PUL 103 | | | | | | | | |
| Bova172_4924 | Predicted | Sialidase (EC 3.2.1.18) | GH33 | 0.25 | 4.1E−01 | −1.55 | 4.5E−10 | −0.53 | 1.6E−01 |
| | PUL 104 | | | | | | | | |
| Bova172_4925 | Predicted | Uncharacterized MFS-type | Trans | −0.25 | 4.4E−01 | −1.39 | 2.9E−07 | 0.29 | 5.4E−01 |
| | PUL 104 | transporter | | | | | | | |
| Bova172_4926 | Predicted | Outer membrane TonB-dependent | Trans | −0.09 | 7.3E−01 | −1.86 | 2.1E−22 | −0.04 | 9.4E−01 |
| | PUL 104 | transporter, SusC family | | | | | | | |
| Bova172_4927 | Predicted | Cell surface glycan-binding | SusD | 1.41 | 6.5E−11 | −2.21 | 2.1E−26 | 0.93 | 5.2E−05 |
| | PUL 104 | lipoprotein, SusD family | | | | | | | |
| Bova172_4928 | Predicted | Sialidase (EC 3.2.1.18) | GH33 | 1.41 | 4.1E−09 | −1.99 | 1.3E−17 | 1.16 | 1.3E−03 |
| | PUL 104 | | | | | | | | |
| Bova172_4929 | Predicted | hypothetical protein | | −0.01 | 9.9E−01 | −1.58 | 5.2E−03 | −0.09 | 1.0E+00 |
| | PUL 104 | | | | | | | | |
| Bova172_4930 | Predicted | PKD | | 0.02 | 9.7E−01 | −1.67 | 9.9E−11 | 0.26 | 5.4E−01 |
| | PUL 104 | | | | | | | | |
| Bova172_4931 | Predicted | beta-glucosidase (EC 3.2.1.21) | GH3 | −0.02 | 9.5E−01 | −1.04 | 2.0E−05 | −0.38 | 4.2E−01 |
| | PUL 104 | | | | | | | | |
| Bova172_4938 | Predicted | RNA polymerase ECF-type sigma | REG | 1.24 | 1.7E−13 | −0.97 | 2.4E−11 | 0.76 | 9.3E−03 |
| | PUL 105 | factor | | | | | | | |
| Bova172_{—}4939 | Predicted | Putative anti-sigma factor | REG | 2.11 | 5.4E−10 | −1.12 | 2.1E−06 | 0.16 | 7.0E−01 |
| | PUL 105 | | | | | | | | |
| Bova172_{—}4940 | Predicted | Outer membrane TonB-dependent | Trans | 1.83 | 1.7E−06 | 0.41 | 1.2E−01 | 0.24 | 5.9E−01 |
| | PUL 105 | transporter, SusC family | | | | | | | |
| Bova172_4941 | Predicted | Cell surface glycan-binding | SusD | 0.56 | 2.3E−01 | −0.10 | 7.6E−01 | 0.11 | 8.4E−01 |
| | PUL 105 | lipoprotein, SusD family | | | | | | | |
| Bova172_4942 | Predicted | Protein-tyrosine-phosphatase (EC | | 0.54 | 2.7E−01 | 0.34 | 4.2E−01 | −0.18 | 7.3E−01 |
| | PUL 105 | 3.1.3.48) | | | | | | | |
| Bova172_4943 | Predicted | Protein tyrosine phosphatase (EC | | 0.62 | 1.9E−01 | −0.46 | 1.3E−01 | −1.10 | 3.9E−02 |
| | PUL 105 | 3.1.3.48) | | | | | | | |
| Bova172_4966 | Predicted | Outer membrane TonB-dependent | Trans | −0.46 | 8.7E−04 | 1.77 | 8.0E−48 | −1.44 | 2.1E−09 |
| | PUL 106 | transporter, SusC family | | | | | | | |
| Bova172_4967 | Predicted | Cell surface glycan-binding | SusD | −0.01 | 9.6E−01 | 1.80 | 1.9E−32 | −1.18 | 8.5E−04 |
| | PUL 106 | lipoprotein, SusD family | | | | | | | |
| Bova172_5355 | Predicted | RNA polymerase ECF-type sigma | REG | −0.60 | 1.5E−02 | 0.35 | 9.0E−02 | −0.48 | 3.2E−01 |
| | PUL 107 | factor | | | | | | | |
| Bova172_5356 | Predicted | hypothetical protein | REG | −0.97 | 1.2E−25 | −2.93 | 1.3E−232 | −1.35 | 1.2E−19 |
| | PUL 107 | | | | | | | | |
| Bova172_5357 | Predicted | Outer membrane TonB-dependent | Trans | −1.34 | 3.9E−28 | −0.86 | 4.9E−14 | −2.24 | 8.5E−45 |
| | PUL 107 | transporter, SusC family | | | | | | | |
| Bova172_5358 | Predicted | Cell surface glycan-binding | SusD | −0.85 | 1.8E−03 | −0.06 | 8.3E−01 | −2.15 | 1.5E−09 |
| | PUL 107 | lipoprotein, SusD family | | | | | | | |
| Bova172_5359 | Predicted | Outer membrane TonB-dependent | Trans | −1.93 | 8.2E−13 | 1.25 | 1.1E−06 | −2.45 | 3.6E−12 |
| | PUL 107 | transporter, SusC family | | | | | | | |
| Bova172_5360 | Predicted | Cell surface glycan-binding | GH16 | −1.26 | 9.7E−06 | 1.30 | 1.6E−06 | −2.51 | 5.6E−10 |
| | PUL 107 | lipoprotein, SusD family | | | | | | | |
| Bova172_5361 | Predicted | beta-glucosidase (EC 3.2.1.21) | GH16-GH3 | −0.89 | 1.1E−03 | 1.26 | 3.8E−07 | −1.61 | 5.1E−06 |
| | PUL 107 | | | | | | | | |
| Bova172_5362 | Predicted | putative purple acid phosphatase | | −1.58 | 9.1E−04 | 1.37 | 4.8E−04 | −2.03 | 2.4E−03 |
| | PUL 107 | | | | | | | | |
| Bova172_5363 | Predicted | hypothetical protein | | −0.53 | 1.6E−01 | 1.38 | 9.9E−05 | −0.75 | 1.3E−01 |
| | PUL 107 | | | | | | | | |
| Bova172_5364 | Predicted | hypothetical protein | | −0.35 | 4.7E−01 | 1.10 | 1.4E−02 | −0.76 | 1.0E+00 |
| | PUL 107 | | | | | | | | |
| Bova172_5389 | Predicted | endo-beta-N- | GH | −0.40 | 9.0E−02 | −2.59 | 5.8E−47 | −1.23 | 6.4E−03 |
| | PUL 108 | acetylglucosaminidase | | | | | | | |
| Bova172_5390 | Predicted | putative patatin-like protein | | −1.84 | 1.0E−11 | −2.53 | 7.5E−36 | −1.20 | 9.9E−03 |
| | PUL 108 | | | | | | | | |
| Bova172_5391 | Predicted | hypothetical protein | | −0.61 | 6.1E−02 | −2.97 | 9.2E−33 | −0.97 | 5.7E−02 |
| | PUL 108 | | | | | | | | |
| Bova172_5392 | Predicted | Cell surface glycan-binding | SusD | −0.25 | 3.5E−01 | −2.77 | 3.2E−53 | −1.23 | 1.7E−03 |
| | PUL 108 | lipoprotein, SusD family | | | | | | | |
| Bova172_5393 | Predicted | Outer membrane TonB-dependent | Trans | 0.56 | 2.8E−05 | −3.46 | 2.3E−191 | −0.03 | 9.1E−01 |
| | PUL 108 | transporter, SusC family | | | | | | | |
| Bova172_{—}5394 | Predicted | Putative anti-sigma factor | REG | 1.49 | 1.5E−26 | −2.91 | 4.7E−91 | 0.03 | 9.2E−01 |
| | PUL 108 | | | | | | | | |
| Bova172_5395 | Predicted | RNA polymerase ECF-type sigma | REG | 1.63 | 5.8E−09 | −0.62 | 8.3E−04 | 1.05 | 1.2E−03 |
| | PUL 108 | factor | | | | | | | |

Table S7B. Corresponding PUL (if present) in B. ovatus 115

| | | | | | Expression | |
|---|---|---|---|---|---|---|
| | | | | | TYG + hemin, ±OF | |
| Gene (in strain 115) | PUL | RAST Annotation | CAZy Classification | % ID | Log2fold | Padj |
| Bova115_4934 | Predicted | RNA polymerase | REG | 99 | 0.01 | 1.0E+00 |
| | PUL 102 | ECF-type sigma factor | | | | |
| Bova115_4935 | Predicted | Putative anti-sigma | REG | 100 | 0.03 | 1.0E+00 |
| | PUL 102 | factor | | | | |
| Bova115_4936 | Predicted | Outer membrane | Trans | 100 | 0.06 | 7.7E−01 |
| | PUL 102 | TonB-dependent | | | | |
| | | transporter, SusC | | | | |
| | | family | | | | |
| Bova115_4937 | Predicted | Cell surface glycan- | SusD | 100 | −0.03 | 1.0E+00 |
| | PUL 102 | binding lipoprotein, | | | | |
| | | SusD family | | | | |
| Bova115_4938 | Predicted | Arylsulfatase (EC | Sulf | 100 | 0.07 | 1.0E+00 |
| | PUL 102 | 3.1.6.1) | | | | |
| Bova115_4939 | Predicted | beta-galactosidase | GH2 | 99 | 0.03 | 9.2E−01 |
| | PUL 102 | (EC 3.2.1.23) | | | | |
| Bova115_5255 | CAZyme | Pectate lyase (EC | PL1_2 | 100 | 0.02 | 1.0E+00 |
| | cluster 7 | 4.2.2.2) | | | | |
| Bova115_5256 | CAZyme | BaeS-type histidine | REG | 100 | 0.05 | 8.6E−01 |
| | cluster 7 | kinase/OmpR-type | | | | |
| | | DNA-binding response | | | | |
| | | regulator | | | | |
| Bova115_5257 | CAZyme | beta-galactosidase | GH2 | 98 | 0.07 | 1.0E+00 |
| | cluster 7 | (EC 3.2.1.23) | | | | |
| Bova115_5258 | CAZyme | hypothetical protein | GH139 | 100 | 0.07 | 1.0E+00 |
| | cluster 7 | | | | | |
| Bova115_5259 | CAZyme | Sialic acid-specific 9- | | 100 | 0.03 | 1.0E+00 |
| | cluster 7 | O-acetylesterase | | | | |
| Bova115_5260 | CAZyme | putative | GH106 | 99 | 0.09 | 6.1E−01 |
| | cluster 7 | glycosyslhydrolase | | | | |
| Bova115_5282 | Predicted | beta-galactosidase | GH137- | 95 | 0.04 | 1.0E+00 |
| | PUL 103 | | CBM57-GH2 | | | |
| Bova115_5283 | Predicted | hypothetical protein | GH138 | 99 | −0.01 | 1.0E+00 |
| | PUL 103 | | | | | |
| Bova115_5284 | Predicted | Two-component | | 99 | −0.05 | 8.5E−01 |
| | PUL 103 | system sensor | | | | |
| | | histidine kinase | | | | |
| Bova115_3452 | Predicted | hypothetical protein | | 53 | −0.01 | 1.0E+00 |
| | PUL 86 | | | | | |
| Bova115_5297 | Predicted | Putative anti-sigma | REG | 71 | −0.02 | 1.0E+00 |
| | PUL 103 | factor | | | | |
| Bova115_5298 | Predicted | RNA polymerase | REG | 84 | 0.72 | 1.5E−02 |
| | PUL 103 | ECF-type sigma factor | | | | |
| Bova115_5305 | | Cytochrome c-type | | 97 | −0.03 | 1.0E+00 |
| | | biogenesis protein | | | | |
| | | DsbD, protein- | | | | |
| | | disulfide reductase | | | | |
| | | (EC 1.8.1.8) | | | | |
| Bova115_5306 | | hypothetical protein | | 96 | 0.01 | 1.0E+00 |
| Bova115_5307 | Predicted | hypothetical protein | Pept | 78 | 0.04 | 1.0E+00 |
| | PUL 104 | | | | | |
| Bova115_5308 | Predicted | hypothetical protein | | 92 | 0.07 | 1.0E+00 |
| | PUL 104 | | | | | |
| Bova115_5309 | Predicted | hypothetical protein | | 77 | −0.01 | 1.0E+00 |
| | PUL 104 | | | | | |
| Bova115_5310 | Predicted | hypothetical protein | | 82 | 0.00 | 1.0E+00 |
| | PUL 104 | | | | | |
| Bova115_5311 | Predicted | hypothetical protein | | 96 | −0.02 | 1.0E+00 |
| | PUL 104 | | | | | |
| Bova115_5312 | Predicted | hypothetical protein | | 96 | 0.01 | 1.0E+00 |
| | PUL 104 | | | | | |
| Bova115_5313 | Predicted | hypothetical protein | | 95 | −0.01 | 1.0E+00 |
| | PUL 104 | | | | | |
| Bova115_5314 | Predicted | hypothetical protein | | 96 | — | — |
| | PUL 104 | | | | | |
| Bova115_5315 | Predicted | hypothetical protein | | 95 | 0.00 | 1.0E+00 |
| | PUL 104 | | | | | |
| Bova115_5316 | Predicted | hypothetical protein | SusD | 99 | 0.02 | 1.0E+00 |
| | PUL 104 | | | | | |
| Bova115_5317 | Predicted | Outer membrane | Trans | 99 | −0.02 | 9.5E−01 |
| | PUL 104 | TonB-dependent | | | | |
| | | transporter, SusC | | | | |
| | | family | | | | |
| Bova115_5330 | CAZyme | Putative large | GH95 | 96 | 0.01 | 1.0E+00 |
| | cluster 9 | secreted protein | | | | |
| | | SCO0341 | | | | |
| Bova115_5331 | CAZyme | Rhamnogalacturonides | GH105 | 97 | −0.03 | 9.0E−01 |
| | cluster 9 | degradation protein | | | | |
| | | RhiN | | | | |
| Bova115_5332 | CAZyme | hypothetical protein | GH140 | 97 | −0.01 | 1.0E+00 |
| | cluster 9 | | | | | |
| Bova115_5333 | CAZyme | alpha-L-rhamnosidase | GH78-GH33 | 97 | −0.03 | 1.0E+00 |
| | cluster 9 | (EC 3.2.1.40) | | | | |
| Bova115_5334 | | Oxidoreductase, | | 91 | −0.06 | 7.9E−01 |
| | | short-chain | | | | |
| | | dehydrogenase/ | | | | |
| | | reductase family | | | | |
| Bova115_5335 | | Serine protease | | 99 | 0.04 | 8.8E−01 |
| Bova115_5350 | Predicted | hypothetical protein | CE | 99 | 0.09 | 6.5E−01 |
| | PUL 105 | | | | | |
| Bova115_5351 | Predicted | Polygalacturonase | GH28 | 99 | 0.03 | 1.0E+00 |
| | PUL 105 | (EC 3.2.1.15) | | | | |
| Bova115_5352 | Predicted | putative secreted | GH78 | 98 | 0.01 | 1.0E+00 |
| | PUL 105 | hydrolase | | | | |
| Bova115_5353 | Predicted | hypothetical protein | GH142- | 99 | −0.02 | 9.4E−01 |
| | PUL 105 | | GH143 | | | |
| Bova115_5354 | Predicted | arabinosidase | GH43_18 | 99 | 0.04 | 1.0E+00 |
| | PUL 105 | | | | | |
| Bova115_5355 | Predicted | hypothetical protein | | 99 | 0.12 | 1.0E+00 |
| | PUL 105 | | | | | |
| Bova115_5356 | Predicted | Pectate lyase (EC | PL1_2 | 99 | 0.00 | 1.0E+00 |
| | PUL 105 | 4.2.2.2) | | | | |
| Bova115_5359 | Predicted | Cell surface glycan- | SusD | 66 | 0.19 | 3.1E−01 |
| | PUL 105 | binding lipoprotein, | | | | |
| | | SusD family | | | | |
| Bova115_5360 | Predicted | Outer membrane | Trans | 73 | 0.00 | 1.0E+00 |
| | PUL 105 | TonB-dependent | | | | |
| | | transporter, SusC | | | | |
| | | family | | | | |
| Bova115_5361 | Predicted | hypothetical protein | | 36 | −0.06 | 1.0E+00 |
| | PUL 105 | | | | | |
| Bova115_5362 | Predicted | hypothetical protein | | 96 | 0.08 | 1.0E+00 |
| | PUL 105 | | | | | |
| Bova115_5363 | Predicted | Alpha-1,2- | GH92 | 97 | −0.01 | 9.8E−01 |
| | PUL 105 | mannosidase | | | | |
| Bova115_5364 | Predicted | 1,4-beta-mannosyl-N- | GH130 | 100 | 0.00 | 1.0E+00 |
| | PUL 105 | acetylglucosamine | | | | |
| | | phosphorylase (EC | | | | |
| | | 2.4.1.320) | | | | |
| Bova115_5365 | Predicted | AmpG protein, beta- | Trans | 99 | −0.19 | 3.4E−01 |
| | PUL 105 | lactamase induction | | | | |
| | | signal transducer | | | | |
| Bova115_5366 | Predicted | beta-hexosaminidase | GH163 | 99 | −0.05 | 8.3E−01 |
| | PUL 105 | precursor | | | | |
| Bova115_5367 | Predicted | Exo-alpha sialidase | | 98 | 0.08 | 6.9E−01 |
| | PUL 105 | | | | | |
| Bova115_5368 | Predicted | Patatin-like protein | | 99 | −0.01 | 1.0E+00 |
| | PUL 105 | | | | | |
| Bova115_5369 | Predicted | Endo-beta-N- | GH18 | 99 | 0.24 | 2.0E−01 |
| | PUL 105 | acetylgucosaminidase | | | | |
| | | F2 | | | | |
| Bova115_5370 | Predicted | Cell surface glycan- | SusD | 100 | 0.00 | 1.0E+00 |
| | PUL 105 | binding lipoprotein, | | | | |
| | | SusD family | | | | |
| Bova115_5371 | Predicted | Outer membrane | Trans | 100 | −0.07 | 7.9E−01 |
| | PUL 105 | TonB-dependent | | | | |
| | | transporter, SusC | | | | |
| | | family | | | | |
| Bova115_5375 | Predicted | Outer membrane | Trans | 99 | 0.42 | 1.1E−01 |
| | PUL 106 | TonB-dependent | | | | |
| | | transporter, SusC | | | | |
| | | family | | | | |
| Bova115_5376 | Predicted | Cell surface glycan- | SusD | 100 | 0.04 | 1.0E+00 |
| | PUL 106 | binding lipoprotein, | | | | |
| | | SusD family | | | | |
| Bova115_5377 | Predicted | Endo-beta-N- | GH18 | 99 | 0.04 | 1.0E+00 |
| | PUL 106 | acetylglucosaminidase | | | | |
| | | F2 | | | | |
| Bova115_5378 | Predicted | putative patatin-like | | 100 | −0.02 | 1.0E+00 |
| | PUL 106 | protein | | | | |
| Bova115_5379 | Predicted | hypothetical protein | | 99 | −0.03 | 1.0E+00 |
| | PUL 106 | | | | | |
| Bova115_5380 | Predicted | Putative anti-sigma | REG | 99 | −0.27 | 9.7E−02 |
| | PUL 106 | factor | | | | |
| Bova115_5381 | Predicted | RNA polymerase | REG | 100 | −0.04 | 1.0E+00 |
| | PUL 106 | ECF-type sigma factor | | | | |
| Bova115_5537 | Predicted | Cell surface glycan- | SusD | 95 | 0.05 | 8.4E−01 |
| | PUL 107 | binding lipoprotein, | | | | |
| | | SusD family | | | | |
| Bova115_5538 | Predicted | Outer membrane | Trans | 99 | −0.09 | 6.9E−01 |
| | PUL 107 | TonB-dependent | | | | |
| | | transporter, SusC | | | | |
| | | family | | | | |
| Bova115_5539 | Predicted | beta-glycosyl | GH20 | 99 | 0.00 | 1.0E+00 |
| | PUL 107 | hydrolase | | | | |
| Bova115_5540 | Predicted | beta-glycosyl | GH20 | 95 | −0.04 | 1.0E+00 |
| | PUL 107 | hydrolase | | | | |
| Bova115_5541 | Predicted | Beta-mannosidase | GH2 | 99 | 0.05 | 8.6E−01 |
| | PUL 107 | (EC 3.2.1.25) | | | | |
| Bova115_5542 | Predicted | Sialidase (EC | GH33 | 99 | −0.06 | 1.0E+00 |
| | PUL 107 | 3.2.1.18) | | | | |
| Bova115_5643 | Predicted | Uncharacterized | Trans | 99 | — | — |
| | PUL 107 | sugar: proton | | | | |
| | | symporter | | | | |
| Bova115_5617 | Predicted | Sialic acid-specific 9- | | 51 | 0.03 | 9.0E−01 |
| | PUL 109 | O-acetylesterase | | | | |
| Bova115_5617 | Predicted | Platelet-activating | | 97 | 0.02 | 1.0E+00 |
| | PUL 110 | factor acetylhydrolase | | | | |
| | | IB gamma subunit (EC | | | | |
| | | 3.1.1.47) | | | | |
| Bova115_5618 | Predicted | hypothetical protein | | 93 | 0.03 | 9.1E−01 |
| | PUL 110 | | | | | |
| Bova115_5619 | Predicted | hypothetical protein | | 95 | 0.01 | 1.0E+00 |
| | PUL 110 | | | | | |
| Bova115_5620 | Predicted | Alpha-N- | GH89 | 95 | 0.00 | 1.0E+00 |
| | PUL 110 | acetylglucosaminidase | | | | |
| | | (EC 3.2.1.50) | | | | |
| Bova115_5621 | Predicted | Alpha-glucosidase | GH97 | 95 | −0.50 | 9.1E−02 |
| | PUL 110 | (EC 3.2.1.20) | | | | |
| Bova115_5622 | Predicted | hypothetical protein | | 94 | 0.03 | 1.0E+00 |
| | PUL 110 | | | | | |
| Bova115_5623 | Predicted | hypothetical protein | CE | 92 | −0.08 | 6.5E−01 |
| | PUL 110 | | | | | |
| Bova115_5624 | Predicted | Alpha-glucosidase | GH97 | 95 | 0.01 | 9.8E−01 |
| | PUL 110 | (EC 3.2.1.20) | | | | |
| Bova115_5625 | Predicted | S-layer related protein | | 98 | −0.01 | 1.0E+00 |
| | PUL 110 | precursor, sialic acid- | | | | |
| | | specific 9-O- | | | | |
| | | acetylesterase | | | | |
| Bova115_5626 | Predicted | hypothetical protein | | 99 | −0.02 | 1.0E+00 |
| | PUL 110 | | | | | |
| Bova115_5627 | Predicted | S-layer related protein | GH | 98 | −0.01 | 1.0E+00 |
| | PUL 110 | precursor, sialic acid- | | | | |
| | | specific 9-O- | | | | |
| | | acetylesterase | | | | |
| Bova115_5628 | Predicted | hypothetical protein | | 99 | −0.01 | 1.0E+00 |
| | PUL 110 | | | | | |
| Bova115_5629 | Predicted | hypothetical protein | | 99 | 0.00 | 1.0E+00 |
| | PUL 110 | | | | | |
| Bova115_5630 | Predicted | Cell surface glycan- | SusD | 100 | 0.00 | 1.0E+00 |
| | PUL 110 | binding lipoprotein, | | | | |
| | | SusD family | | | | |
| Bova115_5631 | Predicted | Outer membrane | Trans | 100 | 0.00 | 1.0E+00 |
| | PUL 110 | TonB-dependent | | | | |
| | | transporter, SusC | | | | |
| | | family | | | | |
| Bova115_9 | Predicted | N-acylglucosamine 2- | EPI | 100 | −0.01 | 1.0E+00 |
| | PUL 1 | epimerase (EC | | | | |
| | | 5.1.3.8) | | | | |
| Bova115_10 | Predicted | Uncharacterized | Trans | 98 | 0.00 | 1.0E+00 |
| | PUL 1 | sugar: proton | | | | |
| | | symporter | | | | |
| Bova115_11 | Predicted | hypothetical protein | | 98 | 0.00 | 1.0E+00 |
| | PUL 1 | | | | | |
| Bova115_12 | Predicted | NTP | | 94 | −0.11 | 4.9E−01 |
| | PUL 1 | pyrophosphohydrolases | | | | |
| | | including oxidative | | | | |
| | | damage repair | | | | |
| | | enzymes | | | | |
| Bova115_2345 | Predicted | BaeS-type histidine | REG | 39 | 0.05 | 1.0E+00 |
| | PUL 45 | kinase/OmpR-type | | | | |
| | | DNA-binding response | | | | |
| | | regulator | | | | |
| Bova115_2340 | Predicted | Glucuronyl hydrolase | GH88 | 69 | −0.03 | 1.0E+00 |
| | PUL 57 | | | | | |
| Bova115_2339 | Predicted | beta-galactosidase | GH2 | 68 | −0.02 | 1.0E+00 |
| | PUL 57 | (EC 3.2.1.23) | | | | |
| Bova115_2350 | Predicted | hypothetical protein | | 35 | 0.00 | 1.0E+00 |
| | PUL 57 | | | | | |
| Bova115_143 | CAZyme | beta-galactosidase | GH35- | 96 | −0.05 | 8.6E−01 |
| | cluster 1 | precursor | CBM32 | | | |
| Bova115_145 | CAZyme | hypothetical protein | GH154 | 97 | 0.01 | 9.9E−01 |
| | cluster 1 | | | | | |
| Bova115_146 | CAZyme | Rhamnogalacturonides | GH105 | 95 | −0.03 | 1.0E+00 |
| | cluster 1 | degradation protein | | | | |
| | | RhiN | | | | |
| Bova115_1012 | Predicted | hypothetical protein | | 45 | 0.02 | 1.0E+00 |
| | PUL 22 | | | | | |
| Bova115_162 | Predicted | hypothetical protein | | 28 | −0.01 | 1.0E+00 |
| | PUL 3 | | | | | |
| Bova115_163 | Predicted | hypothetical protein | Pept | 30 | 0.09 | 6.2E−01 |
| | PUL 3 | | | | | |
| Bova115_164 | Predicted | hypothetical protein | | 33 | −0.03 | 1.0E+00 |
| | PUL 3 | | | | | |
| Bova115_165 | Predicted | hypothetical protein | | 37 | −0.01 | 9.8E−01 |
| | PUL 3 | | | | | |
| Bova115_166 | Predicted | FIG01092053: | | 47 | 0.02 | 1.0E+00 |
| | PUL 3 | hypothetical protein | | | | |
| Bova115_167 | Predicted | Cell surface glycan- | SusD | 44 | 0.03 | 1.0E+00 |
| | PUL 3 | binding lipoprotein, | | | | |
| | | SusD family | | | | |
| Bova115_168 | Predicted | Outer membrane | Trans | 51 | 0.01 | 1.0E+00 |
| | PUL 3 | TonB-dependent | | | | |
| | | transporter, SusC | | | | |
| | | family | | | | |
| Bova115_169 | Predicted | FIG00490691: | | 44 | −0.02 | 9.1E−01 |
| | PUL 3 | hypothetical protein | | | | |
| Bova115_170 | Predicted | hypothetical protein | | 32 | −0.02 | 1.0E+00 |
| | PUL 3 | | | | | |
| Bova115_171 | Predicted | Cell surface glycan- | SusD | 48 | 0.05 | 1.0E+00 |
| | PUL 3 | binding lipoprotein, | | | | |
| | | SusD family | | | | |
| Bova115_172 | Predicted | Outer membrane | Trans | 50 | −0.04 | 8.7E−01 |
| | PUL 3 | TonB-dependent | | | | |
| | | transporter, SusC | | | | |
| | | family | | | | |
| Bova115_175 | CAZyme | BaeS-type histidine | REG | 59 | −0.07 | 7.7E−01 |
| | cluster 2 | kinase/OmpR-type | | | | |
| | | DNA-binding response | | | | |
| | | regulator | | | | |
| Bova115_176 | CAZyme | hypothetical protein | | 77 | −0.10 | 6.4E−01 |
| | cluster 2 | | | | | |
| Bova115_177 | CAZyme | hypothetical protein | GH43_24 | 99 | −0.14 | 4.3E−01 |
| | cluster 2 | | | | | |
| Bova115_178 | CAZyme | hypothetical protein | GH43_24 | 98 | −0.19 | 3.0E−01 |
| | cluster 2 | | | | | |
| Bova115_179 | CAZyme | hypothetical protein | PL27 | 98 | −0.04 | 8.6E−01 |
| | cluster 2 | | | | | |
| Bova115_180 | CAZyme | hypothetical protein | GH43_24 | 99 | −0.01 | 1.0E+00 |
| | cluster 2 | | | | | |
| Bova115_212 | Predicted | hypothetical protein | | 38 | −0.05 | 1.0E+00 |
| | PUL 4 | | | | | |
| Bova115_213 | Predicted | hypothetical protein | | 36 | 0.06 | 1.0E+00 |
| | PUL 4 | | | | | |
| Bova115_214 | Predicted | hypothetical protein | Pept | 49 | 0.02 | 1.0E+00 |
| | PUL 4 | | | | | |
| Bova115_215 | Predicted | hypothetical protein | | 41 | 0.04 | 1.0E+00 |
| | PUL 4 | | | | | |
| Bova115_216 | Predicted | hypothetical protein | | 41 | −0.02 | 1.0E+00 |
| | PUL 4 | | | | | |
| Bova115_217 | Predicted | hypothetical protein | | 30 | −0.04 | 1.0E+00 |
| | PUL 4 | | | | | |
| Bova115_218 | Predicted | hypothetical protein | | 50 | −0.05 | 1.0E+00 |
| | PUL 4 | | | | | |
| Bova115_219 | Predicted | Cell surface glycan- | SusD | 58 | 0.06 | 1.0E+00 |
| | PUL 4 | binding lipoprotein, | | | | |
| | | SusD family | | | | |
| Bova115_220 | Predicted | Outer membrane | Trans | 72 | −0.08 | 1.0E+00 |
| | PUL 4 | TonB-dependent | | | | |
| | | transporter, SusC | | | | |
| | | family | | | | |
| Bova115_231 | Predicted | Uncharacterized MFS- | Trans | 99 | 0.00 | 1.0E+00 |
| | PUL 5 | type transporter | | | | |
| | | Glycerophosphoryl | | | | |
| | | diester | | | | |
| Bova115_232 | Predicted | phosphodiesterase | | 97 | 0.02 | 1.0E−00 |
| | PUL 5 | (EC 3.1.4.46) | | | | |
| Bova115_233 | Predicted | hypothetical protein | | 99 | −0.04 | 8.6E−01 |
| | PUL 5 | | | | | |
| Bova115_234 | Predicted | hypothetical protein | | 100 | 0.01 | 1.0E+00 |
| | PUL 5 | | | | | |
| Bova115_235 | Predicted | Cell surface protein | | 99 | −0.04 | 1.0E+00 |
| | PUL 5 | | | | | |
| Bova115_236 | Predicted | Cell surface glycan- | SusD | 100 | 0.07 | 1.0E+00 |
| | PUL 5 | binding lipoprotein, | | | | |
| | | SusD family | | | | |
| Bova115_237 | Predicted | Outer membrane | Trans | 100 | −0.01 | 9.7E−01 |
| | PUL 5 | TonB-dependent | | | | |
| | | transporter, SusC | | | | |
| | | family | | | | |
| Bova115_238 | Predicted | hypothetical protein | | 100 | −0.01 | 1.0E+00 |
| | PUL 5 | | | | | |
| Bova115_239 | Predicted | Putative anti-sigma | REG | 100 | −0.02 | 9.3E−01 |
| | PUL 5 | factor | | | | |
| Bova115_240 | Predicted | RNA polymerase | REG | 100 | −0.03 | 1.0E+00 |
| | PUL 5 | ECF-type sigma factor | | | | |
| Bova115_278 | Predicted | Glucuronyl hydrolase | GH88 | 95 | −0.04 | 8.6E−01 |
| | PUL 6 | | | | | |
| Bova115_279 | Predicted | putative beta- | GH43_10 | 96 | −0.02 | 1.0E+00 |
| | PUL 6 | xylosidase | | | | |
| Bova115_110 | Predicted | hypothetical protein | | 43 | 0.01 | 1.0E+00 |
| | PUL 2 | | | | | |
| Bova115_282 | Predicted | Cell surface glycan- | SusD | 61 | −0.03 | 1.0E+00 |
| | PUL 6 | binding lipoprotein, | | | | |
| | | SusD family | | | | |
| Bova115_283 | Predicted | Outer membrane | Trans | 63 | 0.04 | 8.8E−01 |
| | PUL 6 | TonB-dependent | | | | |
| | | transporter, SusC | | | | |
| | | family | | | | |
| Bova115_284 | Predicted | hypothetical protein | | 90 | — | — |
| | PUL 6 | | | | | |
| Bova115_285 | Predicted | RNA polymerase | REG | 98 | 0.00 | 1.0E+00 |
| | PUL 6 | ECF-type sigma factor | | | | |
| Bova115_286 | Predicted | DNA-binding response | REG | 100 | −0.11 | 1.0E+00 |
| | PUL 6 | regulator, AraC family | | | | |
| Bova115_289 | Predicted | beta-galactosidase | GH2 | 100 | 0.01 | 1.0E+00 |
| | PUL 7 | (EC 3.2.1.23) | | | | |
| Bova115_290 | Predicted | hypothetical protein | | 100 | 0.00 | 1.0E+00 |
| | PUL 7 | | | | | |
| Bova115_291 | Predicted | hypothetical protein | | 100 | 0.02 | 1.0E+00 |
| | PUL 7 | | | | | |
| Bova115_292 | Predicted | Cell surface glycan- | SusD | 100 | −0.03 | 1.0E+00 |
| | PUL 7 | binding lipoprotein, | | | | |
| | | SusD family | | | | |
| Bova115_293 | Predicted | Outer membrane | Trans | 100 | 0.03 | 1.0E+00 |
| | PUL 7 | TonB-dependent | | | | |
| | | transporter, SusC | | | | |
| | | family | | | | |
| Bova115_294 | Predicted | RNA polymerase | REG | 100 | 0.03 | 1.0E+00 |
| | PUL 7 | ECF-type sigma factor | | | | |
| Bova115_295 | Predicted | hypothetical protein | | 100 | — | — |
| | PUL 7 | | | | | |
| Bova115_296 | Predicted | DNA-binding response | REG | 100 | −0.16 | 1.0E+00 |
| | PUL 7 | regulator, AraC family | | | | |
| Bova115_297 | Predicted | hypothetical protein | PL | 98 | 0.01 | 1.0E+00 |
| | PUL 8 | | | | | |
| Bova115_298 | Predicted | Beta-lactamase class | Pept | 98 | 0.01 | 1.0E+00 |
| | PUL 8 | C-like and penicillin | | | | |
| | | binding proteins | | | | |
| | | (PBPs) superfamily | | | | |
| Bova115_299 | Predicted | Putative glycosyl | GH146 | 94 | 0.01 | 1.0E+00 |
| | PUL 8 | hydrolase of unknown | | | | |
| | | function (DUF1680) | | | | |
| Bova115_319 | Predicted | COG5434 | GH28 | 94 | −0.02 | 1.0E+00 |
| | PUL 9 | Endopygalactorunase | | | | |
| Bova115_320 | Predicted | hypothetical protein | | 99 | 0.01 | 1.0E+00 |
| | PUL 9 | | | | | |
| Bova115_321 | Predicted | hypothetical protein | | 99 | −0.01 | 1.0E+00 |
| | PUL 9 | | | | | |
| Bova115_322 | Predicted | hypothetical protein | | 99 | 0.00 | 1.0E+00 |
| | PUL 9 | | | | | |
| Bova115_323 | Predicted | Cell surface glycan- | SusD | 100 | −0.04 | 1.0E+00 |
| | PUL 9 | Binding lipoprotein, | | | | |
| | | SusD family | | | | |
| Bova115_325 | Predicted | Outer membrane | Trans | 99 | 0.00 | 1.0E+00 |
| | PUL 9 | TonB-dependent | | | | |
| | | transporter, SusC | | | | |
| | | family | | | | |
| Bova115_326 | Predicted | RNA polymerase | REG | 100 | 0.03 | 1.0E+00 |
| | PUL 9 | ECF-type sigma factor | | | | |
| Bova115_327 | Predicted | hypothetical protein | | 100 | 0.04 | 8.8E−01 |
| | PUL 9 | | | | | |
| Bova115_328 | Predicted | BaeS-type histidine | REG | 100 | 0.13 | 5.4E−01 |
| | PUL 9 | kinase/OmpR-type | | | | |
| | | DNA-binding response | | | | |
| | | regulator | | | | |
| Bova115_329 | Predicted | COG5434 | CBM6 | 100 | 0.06 | 7.9E−01 |
| | PUL 9 | Endopygalactorunase | | | | |
| Bova115_330 | Predicted | COG5434 | GH28 | 100 | 0.01 | 1.0E+00 |
| | PUL 9 | Endopygalactorunase | | | | |
| Bova115_356 | Predicted | Outer membrane | Trans | 42 | 0.02 | 9.4E−01 |
| | PUL 10 | TonB-dependent | | | | |
| | | transporter, SusC | | | | |
| | | family | | | | |
| Bova115_357 | Predicted | Iron siderophore | REG | 99 | 0.01 | 1.0E+00 |
| | PUL 10 | sensor protein | | | | |
| Bova115_358 | Predicted | RNA polymerase | REG | 100 | 0.02 | 1.0E+00 |
| | PUL 10 | ECF-type sigma factor | | | | |
| Bova115_503 | Predicted | Outer membrane | Trans | 49 | 0.01 | 9.8E−01 |
| | PUL 12 | TonB-dependent | | | | |
| | | transporter, SusC | | | | |
| | | family | | | | |
| Bova115_504 | Predicted | Putative anti-sigma | REG | 74 | 0.13 | 1.0E+00 |
| | PUL 12 | factor | | | | |
| Bova115_507 | Predicted | RNA polymerase | REG | 92 | −0.01 | 1.0E+00 |
| | PUL 12 | ECF-type sigma factor | | | | |
| Bova115_516 | Predicted | Acetyltransferase, | | 100 | −0.02 | 1.0E+00 |
| | PUL 13 | GNAT family | | | | |
| Bova115_517 | Predicted | 23S rRNA | | 100 | 0.04 | 1.0E+00 |
| | PUL 13 | (adenine(1618)-N(6))- | | | | |
| | | methyltransferase (EC | | | | |
| | | 2.1.1.181) | | | | |
| Bova115_518 | Predicted | Two-component | | 99 | 0.00 | 1.0E+00 |
| | PUL 13 | transcriptional | | | | |
| | | response regulator, | | | | |
| | | LuxR family | | | | |
| Bova115_519 | Predicted | Cell surface glycan- | SusD | 99 | −0.02 | 1.0E+00 |
| | PUL 13 | binding lipoprotein, | | | | |
| | | SusD family | | | | |
| Bova115_520 | Predicted | Outer membrane | Trans | 99 | −0.06 | 1.0E+00 |
| | PUL 13 | TonB-dependent | | | | |
| | | transporter, SusC | | | | |
| | | family | | | | |
| Bova115_545 | Predicted | heparin lyase I | PL13 | 99 | 0.00 | 1.0E+00 |
| | PUL 14 | precursor EC: 4.2.2.7) | | | | |
| Bova115_546 | Predicted | hypothetical protein | | 98 | 0.06 | 8.0E−01 |
| | PUL 14 | | | | | |
| Bova115_547 | Predicted | hypothetical protein | REG | 99 | −0.04 | 8.8E−01 |
| | PUL 14 | | | | | |
| Bova115_548 | Predicted | hypothetical protein | GH147 | 98 | 0.05 | 1.0E+00 |
| | PUL 14 | | | | | |
| Bova115_549 | Predicted | Outer membrane | Trans | 93 | −0.04 | 8.8E−01 |
| | PUL 14 | TonB-dependent | | | | |
| | | transporter, SusC | | | | |
| | | family | | | | |
| Bova115_550 | Predicted | Cell surface glycan- | SusD | 83 | 0.02 | 1.0E+00 |
| | PUL 14 | binding lipoprotein, | | | | |
| | | SusD family | | | | |
| Bova115_551 | Predicted | hypothetical protein | | 49 | 0.00 | 1.0E+00 |
| | PUL 14 | | | | | |
| Bova115_552 | Predicted | arabinogalactan endo- | GH53 | 66 | −0.01 | 1.0E+00 |
| | PUL 14 | 1,4-beta-galactosidase | | | | |
| Bova115_553 | Predicted | beta-galactosidase | GH2 | 99 | 0.07 | 7.7E−01 |
| | PUL 14 | (EC 3.2.1.23) | | | | |
| Bova115_564 | Predicted | BaeS-type histidine | REG | 99 | 0.02 | 9.2E−01 |
| | PUL 15 | kinase/OmpR-type | | | | |
| | | DNA-binding response | | | | |
| | | regulator | | | | |
| Bova115_566 | Predicted | Phenylalanyl-tRNA | PL12_2 | 54 | −0.08 | 7.3E−01 |
| | PUL 15 | Synthetase beta chain | | | | |
| | | (EC 6.1.1.20) | | | | |
| Bova115_567 | Predicted | hypothetical protein | | 52 | −0.03 | 9.1E−01 |
| | PUL 15 | | | | | |
| Bova115_568 | Predicted | Outer membrane | Trans | 80 | −0.20 | 1.9E−01 |
| | PUL 15 | TonB-dependent | | | | |
| | | transporter, SusC | | | | |
| | | family | | | | |
| Bova115_569 | Predicted | Cell surface glycan- | SusD | 72 | −0.08 | 74.E−01 |
| | PUL 15 | binding lipoprotein, | | | | |
| | | SusD family | | | | |
| Bova115_570 | Predicted | Glucuronyl hydrolase | GH88 | 96 | −0.02 | 1.0E+00 |
| | PUL 15 | | | | | |
| Bova115_571 | Predicted | heparinase III protein | PL12_2 | 99 | −0.07 | 1.0E+00 |
| | PUL 15 | | | | | |
| Bova115_572 | Predicted | Mucin-desulfating | Sulf | 100 | 0.00 | 1.0E+00 |
| | PUL 15 | sulfatase | | | | |
| Bova115_573 | | hypothetical protein | | 99 | −0.01 | 1.0E+00 |
| Bova115_574 | | ROK family protein | ROK | 95 | 0.03 | 1.0E+00 |
| | | (putative glucokinase) | | | | |
| Bova115_575 | | Hypothetical sugar | Trans | 98 | −0.06 | 1.0E+00 |
| | | permease | | | | |
| Bova115_576 | | hypothetical protein | PL15_2 | 99 | 0.01 | 1.0E+00 |
| Bova115_751 | Predicted | hypothetical protein | | 99 | −0.03 | 1.0E+00 |
| | PUL 16 | | | | | |
| Bova115_752 | Predicted | Cell surface glycan- | SusD | 100 | −0.05 | 1.0E+00 |
| | PUL 16 | binding lipoprotein, | | | | |
| | | SusD family | | | | |
| Bova115_753 | Predicted | Outer membrane | Trans | 100 | 0.00 | 9.9E−01 |
| | PUL 16 | TonB-dependent | | | | |
| | | transporter, SusC | | | | |
| | | family | | | | |
| Bova115_773 | Predicted | Fructokinase (EC | | 98 | 0.05 | 1.0E+00 |
| | PUL 17 | 2.7.1.4) | | | | |
| Bova115_774 | Predicted | Alcohol | | 99 | −0.01 | 1.0E+00 |
| | PUL 17 | dehydrogenase (EC | | | | |
| | | 1.1.1.1) | | | | |
| Bova115_775 | Predicted | hypothetical protein | | 98 | 0.04 | 1.0E+00 |
| | PUL 17 | | | | | |
| Bova115_776 | Predicted | Multiple polyol-specific | | 100 | 0.02 | 1.0E+00 |
| | PUL 17 | dehydrogenase (EC | | | | |
| | | 1.1.1.—) | | | | |
| Bova115_777 | Predicted | hypothetical protein | | 99 | −0.02 | 1.0E+00 |
| | PUL 17 | | | | | |
| Bova115_778 | Predicted | hypothetical protein | | 100 | 0.04 | 1.0E+00 |
| | PUL 17 | | | | | |
| Bova115_779 | Predicted | hypothetical protein | | 99 | 0.01 | 1.0E+00 |
| | PUL 17 | | | | | |
| Bova115_780 | Predicted | hypothetical protein | | 100 | 0.01 | 1.0E+00 |
| | PUL 17 | | | | | |
| Bova115_781 | Predicted | hypothetical protein | | 100 | −0.03 | 1.0E+00 |
| | PUL 17 | | | | | |
| Bova115_782 | Predicted | Cell surface glycan- | SusD | 99 | −0.04 | 1.0E+00 |
| | PUL 17 | binding lipoprotein, | | | | |
| | | SusD family | | | | |
| Bova115_783 | Predicted | Outer membrane | Trans | 100 | −0.01 | 1.0E+00 |
| | PUL 17 | TonB-dependent | | | | |
| | | transporter, SusC | | | | |
| | | family | | | | |
| Bova115_784 | Predicted | hypothetical protein | | 96 | 0.07 | 1.0E+00 |
| | PUL 17 | | | | | |
| Bova115_785 | Predicted | hypothetical protein | | 99 | 0.02 | 1.0E+00 |
| | PUL 17 | | | | | |
| Bova115_786 | Predicted | FAD dependent | | 99 | −0.02 | 1.0E+00 |
| | PUL 17 | oxidoreductase | | | | |
| Bova115_840 | Predicted | hypothetical protein | | 100 | −0.08 | 1.0E+00 |
| | PUL 18 | | | | | |
| Bova115_841 | Predicted | hypothetical protein | | 100 | −0.01 | 1.0E+00 |
| | PUL 18 | | | | | |
| Bova115_842 | Predicted | Cell surface glycan- | SusD | 100 | 0.01 | 1.0E+00 |
| | PUL 18 | binding lipoprotein, | | | | |
| | | SusD family | | | | |
| Bova115_843 | Predicted | Outer membrane | Trans | 99 | 0.10 | 5.6E−01 |
| | PUL 18 | TonB-dependent | | | | |
| | | transporter, SusC | | | | |
| | | family | | | | |
| Bova115_844 | Predicted | Putative anti-sigma | REG | 99 | 0.06 | 7.7E−01 |
| | PUL 18 | factor | | | | |
| Bova115_845 | Predicted | RNA polymerase | REG | 99 | −0.04 | 1.0E+00 |
| | PUL 18 | ECF-type sigma factor | | | | |
| Bova115_856 | Predicted | Putative anti-sigma | REG | 95 | −0.04 | 1.0E+00 |
| | PUL 19 | factor | | | | |
| Bova115_857 | Predicted | hypothetical protein | | 98 | — | — |
| | PUL 19 | | | | | |
| Bova115_858 | Predicted | RNA polymerase | REG | 100 | 0.00 | 1.0E+00 |
| | PUL 19 | ECF-type sigma factor | | | | |
| Bova115_877 | Predicted | RNA polymerase | REG | 100 | 0.01 | 9.5E−01 |
| | PUL 20 | sigma-70 factor | | | | |
| Bova115_878 | Predicted | Putative anti-sigma | REG | 100 | 0.00 | 1.0E+00 |
| | PUL 20 | factor | | | | |
| Bova115_879 | Predicted | Outer membrane | Trans | 91 | −0.06 | 1.0E+00 |
| | PUL 20 | TonB-dependent | | | | |
| | | transporter, SusC | | | | |
| | | family | | | | |
| Bova115_880 | Predicted | Cell surface glycan- | SusD | 87 | −0.01 | 1.0E+00 |
| | PUL 20 | binding lipoprotein, | | | | |
| | | SusD family | | | | |
| Bova115_881 | Predicted | hypothetical protein | | 86 | 0.03 | 1.0E+00 |
| | PUL 20 | | | | | |
| Bova115_882 | Predicted | Thioredoxin family | | 92 | 0.00 | 1.0E+00 |
| | PUL 20 | protein | | | | |
| Bova115_883 | Predicted | hypothetical protein | Pept | 94 | 0.01 | 1.0E+00 |
| | PUL 20 | | | | | |
| Bova115_884 | Predicted | hypothetical protein | | 100 | −0.19 | 2.9E−01 |
| | PUL 20 | | | | | |
| Bova115_885 | Predicted | hypothetical protein | | 100 | 0.00 | 1.0E+00 |
| | PUL 20 | | | | | |
| Bova115_886 | Predicted | Thioredoxin | | 100 | 0.00 | 1.0E+00 |
| | PUL 20 | | | | | |
| Bova115_887 | Predicted | beta-glycosyl | GH20 | 100 | −0.02 | 9.2E−01 |
| | PUL 20 | hydrolase | | | | |
| Bova115_945 | CAZyme | alpha-N- | GH109 | 100 | 0.04 | 8.6E−01 |
| | cluster 3 | acetylgalactosaminidase | | | | |
| | | (EC 3.2.1.49) | | | | |
| Bova115_946 | CAZyme | Oxidoreductase, | GH109 | 100 | 0.05 | 7.7E−01 |
| | cluster 3 | Gfo/Idh/MocA family | | | | |
| Bova115_947 | CAZyme | Uncharacterized | | 100 | 0.42 | 1.0E−01 |
| | cluster 3 | membrane-anchored | | | | |
| | | protein, YitT family | | | | |
| Bova115_948 | CAZyme | beta-galactosidase | GH2 | 100 | 0.07 | 7.6E−01 |
| | cluster 3 | (EC 3.2.1.23) | | | | |
| Bova115_949 | CAZyme | MdsC protein | | 100 | −0.03 | 1.0E+00 |
| | cluster 3 | | | | | |
| Bova115_994 | | Rhamnogalacturonides | GH105 | 98 | 0.05 | 1.0E+00 |
| | | degradation protein | | | | |
| | | RhiN | | | | |
| Bova115_995 | | BaeS-type histidine | REG | 99 | 0.02 | 9.6E−01 |
| | | kinase/OmpR-type | | | | |
| | | DNA-binding response | | | | |
| | | regulator | | | | |
| Bova115_1013 | Predicted | Polygalacturonase | GH28 | 100 | −0.01 | 1.0E+00 |
| | PUL 22 | (EC 3.2.1.15) | | | | |
| Bova115_1014 | Predicted | hypothetical protein | GH | 99 | 0.05 | 1.0E+00 |
| | PUL 22 | | | | | |
| Bova115_1015 | Predicted | Polygalacturonase | | 99 | 0.02 | 1.0E+00 |
| | PUL 22 | (EC 3.2.1.15) | | | | |
| Bova115_1016 | Predicted | hypothetical protein | SusD | 100 | 0.05 | 1.0E+00 |
| | PUL 22 | | | | | |
| Bova115_1017 | Predicted | Outer membrane | Trans | 100 | −0.06 | 1.0E+00 |
| | PUL 22 | TonB-dependent | | | | |
| | | transporter, SusC | | | | |
| | | family | | | | |
| Bova115_1018 | Predicted | hypothetical protein | | 100 | −0.01 | 1.0E+00 |
| | PUL 22 | | | | | |
| Bova115_1019 | Predicted | hypothetical protein | | 100 | 0.00 | 1.0E+00 |
| | PUL 22 | | | | | |
| Bova115_1020 | Predicted | Cell surface glycan- | SusD | 98 | −0.01 | 1.0E+00 |
| | PUL 22 | binding lipoprotein, | | | | |
| | | SusD family | | | | |
| Bova115_1021 | Predicted | Outer membrane | Trans | 100 | −0.05 | 1.0E+00 |
| | PUL 22 | TonB-dependent | | | | |
| | | transporter, SusC | | | | |
| | | family | | | | |
| Bova115_1022 | Predicted | hypothetical protein | | 99 | −0.03 | 1.0E+00 |
| | PUL 22 | | | | | |
| Bova115_1023 | Predicted | hypothetical protein | | 100 | 0.00 | 1.0E+00 |
| | PUL 22 | | | | | |
| Bova115_1024 | Predicted | hypothetical protein | PL1_2 | 99 | 0.00 | 1.0E+00 |
| | PUL 22 | | | | | |
| Bova115_1025 | Predicted | hypothetical protein | | 95 | −0.03 | 9.0E−01 |
| | PUL 22 | | | | | |
| Bova115_1026 | Predicted | Rhamnogalacturonides | GH105 | 99 | 0.02 | 1.0E+00 |
| | PUL 22 | degradation protein | | | | |
| | | RhiN | | | | |
| Bova115_1027 | Predicted | Exopolygalacturonase | GH28 | 98 | 0.01 | 1.0E+00 |
| | PUL 22 | precursor | | | | |
| Bova115_1028 | Predicted | hypothetical protein | | 98 | — | — |
| | PUL 22 | | | | | |
| Bova115_1029 | Predicted | hypothetical protein | | 96 | 0.02 | 1.0E+00 |
| | PUL 22 | | | | | |
| Bova115_1030 | Predicted | pectate lyase L | PL9_1 | 98 | 0.07 | 1.0E+00 |
| | PUL 22 | precursor | | | | |
| Bova115_1031 | Predicted | exo-poly-alpha-D- | GH28 | 100 | −0.05 | 1.0E+00 |
| | PUL 22 | galacturonosidase | | | | |
| | | precursor | | | | |
| Bova115_1032 | Predicted | Rhamnogalacturonan | PL11_1 | 98 | −0.21 | 2.8E−01 |
| | PUL 22 | endolyase (EC | | | | |
| | | 4.2.2.23) | | | | |
| Bova115_1033 | Predicted | BaeS-type histidine | REG | 99 | −0.02 | 9.4E−01 |
| | PUL 23 | kinase/OmpR-type | | | | |
| | | DNA-binding response | | | | |
| | | regulator | | | | |
| Bova115_1035 | Predicted | beta-galactosidase | GH2 | 99 | −0.03 | 9.0E−01 |
| | PUL 23 | | | | | |
| Bova115_1036 | Predicted | hypothetical protein | CE | 100 | 0.00 | 1.0E+00 |
| | PUL 23 | | | | | |
| Bova115_1037 | Predicted | Outer membrane | Trans | 99 | −0.03 | 9.0E−01 |
| | PUL 23 | TonB-dependent | | | | |
| | | transporter, SusC | | | | |
| | | family | | | | |
| Bova115_1038 | Predicted | Cell surface glycan- | SusD | 100 | 0.04 | 8.6E−01 |
| | PUL 23 | binding lipoprotein, | | | | |
| | | SusD family | | | | |
| Bova115_1039 | Predicted | hypothetical protein | | 100 | — | — |
| | PUL 23 | | | | | |
| Bova115_1040 | Predicted | hypothetical protein | | 100 | 0.78 | 4.5E−02 |
| | PUL 23 | | | | | |
| Bova115_1043 | Predicted | hypothetical protein | | 100 | −0.11 | 1.0E+00 |
| | PUL 23 | | | | | |
| Bova115_1044 | Predicted | rhamnogalacturonan | CE12 | 99 | 0.24 | 1.0E+00 |
| | PUL 23 | acetylesterase | | | | |
| Bova115_1045 | Predicted | hypothetical protein | | 99 | −0.02 | 9.3E−01 |
| | PUL 23 | | | | | |
| Bova115_1046 | Predicted | hypothetical protein | | 99 | −0.01 | 1.0E+00 |
| | PUL 23 | | | | | |
| Bova115_1047 | Predicted | hypothetical protein | | 100 | 0.06 | 1.0E+00 |
| | PUL 23 | | | | | |
| Bova115_1048 | Predicted | Rhamnogalacturonides | GH105 | 99 | −0.03 | 1.0E+00 |
| | PUL 23 | degradation protein | | | | |
| | | RhiN | | | | |
| Bova115_1049 | Predicted | Polysaccharide | CE | 99 | 0.00 | 1.0E+00 |
| | PUL 23 | deacetylase | | | | |
| Bova115_1050 | Predicted | BaeS-type histidine | REG | 99 | −0.01 | 1.0E+00 |
| | PUL 23 | kinase/OmpR-type | | | | |
| | | DNA-binding response | | | | |
| | | regulator | | | | |
| Bova115_1051 | Predicted | L-rhamnose | | 99 | 0.01 | 1.0E+00 |
| | PUL 23 | mutarotase (EC | | | | |
| | | 5.1.3.32) | | | | |
| Bova115_1052 | Predicted | Rhamnogalacturonides | GH105 | 99 | −0.01 | 1.0E+00 |
| | PUL 23 | degradation protein | | | | |
| | | RhiN | | | | |
| Bova115_1053 | Predicted | Rhamnogalacturonan | PL11_1 | 99 | −0.01 | 1.0E+00 |
| | PUL 23 | endolyase (EC | | | | |
| | | 4.2.2.23) | | | | |
| Bova115_1054 | Predicted | Rhamnogalacturonides | GH105 | 99 | 0.01 | 1.0E+00 |
| | PUL 23 | degradation protein | | | | |
| | | RhiN | | | | |
| Bova115_1055 | Predicted | rhamnogalacturonan | CE12-CE12 | 99 | 0.01 | 1.0E+00 |
| | PUL 23 | acetylesterase | | | | |
| Bova115_1056 | Predicted | hypothetical protein | PL26 | 93 | 0.02 | 1.0E+00 |
| | PUL 23 | | | | | |
| Bova115_1057 | Predicted | hypothetical protein | PL11_1 | 88 | 0.09 | 1.0E+00 |
| | PUL 23 | | | | | |
| Bova115_1058 | Predicted | Cell surface glycan- | SusD | 72 | 0.05 | 1.0E+00 |
| | PUL 23 | binding lipoprotein, | | | | |
| | | SusD family | | | | |
| Bova115_1059 | Predicted | Outer membrane | Trans | 79 | −0.04 | 9.0E−01 |
| | PUL 23 | TonB-dependent | | | | |
| | | transporter, SusC | | | | |
| | | family | | | | |
| Bova115_1060 | Predicted | Cell surface glycan- | SusD | 69 | 0.00 | 1.0E+00 |
| | PUL 23 | binding lipoprotein, | | | | |
| | | SusD family | | | | |
| Bova115_1061 | Predicted | Outer membrane | Trans | 76 | 0.05 | 1.0E+00 |
| | PUL 23 | TonB-dependent | | | | |
| | | transporter, SusC | | | | |
| | | family | | | | |
| Bova115_1062 | Predicted | hypothetical protein | | 55 | 0.01 | 1.0E+00 |
| | PUL 23 | | | | | |
| Bova115_1063 | Predicted | hypothetical protein | | 68 | 0.04 | 1.0E+00 |
| | PUL 23 | | | | | |
| Bova115_1065 | Predicted | integrase | | 91 | 0.06 | 8.0E−01 |
| | PUL 23 | | | | | |
| Bova115_1066 | Predicted | Outer membrane | Trans | 100 | 0.03 | 1.0E+00 |
| | PUL 24 | TonB-dependent | | | | |
| | | transporter, SusC | | | | |
| | | family | | | | |
| Bova115_1067 | Predicted | Cell surface glycan- | SusD | 100 | 0.03 | 8.8E−01 |
| | PUL 24 | binding lipoprotein, | | | | |
| | | SusD family | | | | |
| Bova115_1068 | Predicted | hypothetical protein | | 100 | −0.02 | 1.0E+00 |
| | PUL 24 | | | | | |
| Bova115_1069 | Predicted | Polygalacturonase | GH28 | 100 | −0.03 | 1.0E+00 |
| | PUL 24 | (EC 3.2.1.15) | | | | |
| Bova115_1070 | Predicted | hypothetical protein | | 100 | 0.00 | 1.0E+00 |
| | PUL 24 | | | | | |
| Bova115_1071 | Predicted | beta-galactosidase | GH42 | 99 | −0.01 | 1.0E+00 |
| | PUL 24 | (EC 3.2.1.23) | | | | |
| Bova115_1072 | Predicted | beta-galactosidase | GH43_18- | 100 | 0.05 | 1.0E+00 |
| | PUL 24 | (EC 3.2.1.23) | CBM32- | | | |
| | | | GH43_34 | | | |
| Bova115_1073 | Predicted | beta-galactosidase | GH2 | 100 | −0.01 | 1.0E+00 |
| | PUL 24 | (EC 3.2.1.23) | | | | |
| Bova115_1074 | Predicted | rhamnogalacturonan | CE12 | 100 | 0.02 | 1.0E+00 |
| | PUL 24 | acetylesterase | | | | |
| Bova115_1075 | Predicted | exo-poly-alpha-D- | GH28 | 100 | −0.06 | 8.5E−01 |
| | PUL 24 | galacturonosidase | | | | |
| | | precursor | | | | |
| Bova115_1076 | Predicted | hypothetical protein | | 100 | 0.00 | 1.0E+00 |
| | PUL 24 | | | | | |
| Bova115_1077 | Predicted | Exo-poly-alpha-D- | GH28 | 99 | −0.03 | 1.0E+00 |
| | PUL 24 | galacturonosidase | | | | |
| | | precursor (EC | | | | |
| | | 3.2.1.82) | | | | |
| Bova115_1078 | Predicted | Predicted alpha-L- | GH106 | 96 | 0.02 | 1.0E+00 |
| | PUL 24 | rhamnosidase | | | | |
| Bova115_1079 | Predicted | Cystathionine beta- | | 96 | −0.02 | 9.5E−01 |
| | PUL 24 | lyase (EC 4.4.1.8) | | | | |
| Bova115_1080 | Predicted | hypothetical protein | | 99 | −0.03 | 1.0E+00 |
| | PUL 24 | | | | | |
| Bova115_1090 | | BaeS-type histidine | REG | 100 | −0.06 | 7.3E−01 |
| | | kinase/OmpR-type | | | | |
| | | DNA-binding response | | | | |
| | | regulator | | | | |
| Bova115_1091 | | SAM-dependent | | 98 | 0.10 | 1.0E+00 |
| | | methyltransferase | | | | |
| Bova115_1092 | | beta-glucosidase (EC | GH3 | 100 | 0.00 | 1.0E+00 |
| | | 3.2.1.21) | | | | |
| Bova115_1098 | Predicted | Polygalacturonase | GH28 | 100 | −0.02 | 1.0E+00 |
| | PUL 25 | (EC 3.2.1.15) | | | | |
| Bova115_1099 | Predicted | hypothetical protein | CE8 | 99 | −0.01 | 1.0E+00 |
| | PUL 25 | | | | | |
| Bova115_1100 | Predicted | hypothetical protein | | 100 | 0.04 | 1.0E+00 |
| | PUL 25 | | | | | |
| Bova115_1101 | Predicted | hypothetical protein | | 100 | 0.00 | 1.0E+00 |
| | PUL 25 | | | | | |
| Bova115_1102 | Predicted | Cell surface glycan- | SusD | 100 | 0.05 | 1.0E+00 |
| | PUL 25 | binding lipoprotein, | | | | |
| | | SusD family | | | | |
| Bova115_1103 | Predicted | Outer membrane | Trans | 99 | 0.02 | 1.0E+00 |
| | PUL 25 | TonB-dependent | | | | |
| | | transporter, SusC | | | | |
| | | family | | | | |
| Bova115_1104 | Predicted | Pectate lyase (EC | PL1_2 | 99 | −0.03 | 1.0E+00 |
| | PUL 25 | 4.2.2.2) | | | | |
| Bova115_1105 | Predicted | hypothetical protein | | 97 | 0.01 | 1.0E+00 |
| | PUL 25 | | | | | |
| Bova115_1106 | Predicted | hypothetical protein | | 99 | — | — |
| | PUL 25 | | | | | |
| Bova115_1107 | Predicted | Pectate lyase (EC | PL1_2 | 100 | −0.03 | 9.0E−01 |
| | PUL 25 | 4.2.2.2) | | | | |
| Bova115_1108 | Predicted | Two-component | | 100 | −0.02 | 1.0E+00 |
| | PUL 25 | system response | | | | |
| | | regulator without | | | | |
| | | neighboring kinase | | | | |
| Bova115_1109 | Predicted | Transmembrane | | 100 | 0.05 | 1.0E+00 |
| | PUL 25 | protein | | | | |
| Bova115_1110 | Predicted | FIG00409296: | | 100 | 0.03 | 1.0E+00 |
| | PUL 25 | hypothetical protein | | | | |
| Bova115_1111 | Predicted | Outer membrane | Trans | 100 | 0.00 | 1.0E+00 |
| | PUL 25 | TonB-dependent | | | | |
| | | transporter, SusS | | | | |
| | | family | | | | |
| Bova115_1112 | Predicted | Cell surface glycan- | SusD | 100 | 0.00 | 1.0E+00 |
| | PUL 25 | binding lipoprotein, | | | | |
| | | SusD family | | | | |
| Bova115_1113 | Predicted | hypothetical protein | | 98 | 0.00 | 1.0E+00 |
| | PUL 25 | | | | | |
| Bova115_1114 | Predicted | hypothetical protein | CE8 | 100 | 0.01 | 1.0E+00 |
| | PUL 25 | | | | | |
| Bova115_1115 | Predicted | Pectate lyase (EC | PL1_2 | 98 | 0.04 | 1.0E+00 |
| | PUL 25 | 4.2.2.2) | | | | |
| Bova115_1116 | Predicted | Pectate lyase (EC | PL1_2 | 99 | 0.01 | 1.0E+00 |
| | PUL 25 | 4.2.2.2) | | | | |
| Bova115_1117 | Predicted | Pectate lyase (EC | PL1_2 | 99 | −0.05 | 1.0E+00 |
| | PUL 25 | 4.2.2.2) | | | | |
| Bova115_1118 | Predicted | BaeS-type histidine | REG | 99 | 0.02 | 9.3E−01 |
| | PUL 25 | kinase/OmpR-type | | | | |
| | | DNA-binding response | | | | |
| | | regulator | | | | |
| Bova115_1119 | Predicted | rhamnogalacturonan | CE12-CE8 | 99 | 0.04 | 1.0E+00 |
| | PUL 25 | acetylesterase | | | | |
| Bova115_1120 | Predicted | hypothetical protein | | 93 | — | — |
| | PUL 25 | | | | | |
| Bova115_1121 | Predicted | rhamnogalacturonan | CE8 | 99 | −0.06 | 1.0E+00 |
| | PUL 25 | acetylesterase | | | | |
| Bova115_1122 | Predicted | Rhamnogalacturonides | GH105 | 99 | 0.03 | 1.0E+00 |
| | PUL 25 | degradation protein | | | | |
| | | RhiN | | | | |
| Bova115_1134 | Predicted | hypothetical protein | CE | 99 | −0.03 | 1.0E+00 |
| | PUL 26 | | | | | |
| Bova115_1136 | Predicted | FIG01423360: | GH130 | 99 | 0.04 | 1.0E+00 |
| | PUL 26 | glycoside hydrolase | | | | |
| Bova115_1137 | Predicted | Putative alpha-1,2- | GH92 | 100 | −0.03 | 1.0E+00 |
| | PUL 26 | mannosidase | | | | |
| Bova115_1138 | Predicted | Alpha-1,2- | GH92 | 99 | −0.02 | 1.0E+00 |
| | PUL 26 | mannosidase | | | | |
| Bova115_1139 | Predicted | Sialic acid-specific 9- | | 100 | −0.01 | 1.0E+00 |
| | PUL 26 | O-acetylesterase | | | | |
| Bova115_1140 | Predicted | hypothetical protein | | 99 | −0.07 | 7.5E−01 |
| | PUL 26 | | | | | |
| Bova115_1141 | Predicted | Cell surface glycan- | SusD | 97 | 0.02 | 1.0E+00 |
| | PUL 26 | binding lipoprotein, | | | | |
| | | SusD family | | | | |
| Bova115_1142 | Predicted | hypothetical protein | | 96 | — | — |
| | PUL 26 | | | | | |
| Bova115_1143 | Predicted | hypothetical protein | | 91 | −0.01 | 1.0E+00 |
| | PUL 26 | | | | | |
| Bova115_1144 | Predicted | Outer membrane | | 100 | −0.01 | 1.0E+00 |
| | PUL 26 | TonB-dependent | | | | |
| | | transporter, SusC | | | | |
| | | family | | | | |
| Bova115_1145 | Predicted | Cell Surface glycan- | SusD | 99 | 0.01 | 1.0E+00 |
| | PUL 26 | binding lipoprotein, | | | | |
| | | SusD family | | | | |
| Bova115_1146 | Predicted | Outer membrane | Trans | 100 | 0.00 | 1.0E+00 |
| | PUL 26 | TonB-dependent | | | | |
| | | transporter, SusC | | | | |
| | | family | | | | |
| Bova115_1148 | Predicted | Alpha-glucosidase | GH31 | 100 | −0.06 | 1.0E+00 |
| | PUL 26 | (EC 3.2.1.20) | | | | |
| Bova115_1149 | Predicted | hypothetical protein | | 100 | — | — |
| | PUL 26 | | | | | |
| Bova115_1150 | Predicted | alpha-rhamnosidase | GH78 | 93 | 0.03 | 9.0E−01 |
| | PUL 26 | | | | | |
| Bova115_1151 | Predicted | hypothetical protein | CE | 98 | −0.05 | 1.0E+00 |
| | PUL 26 | | | | | |
| Bova115_1152 | Predicted | Beta-mannosidase | CBM32- | 98 | 0.01 | 1.0E+00 |
| | PUL 26 | (EC 3.2.1.25) | GH2 | | | |
| Bova115_1153 | Predicted | Alpha-1,2- | GH92 | 99 | −0.05 | 1.0E+00 |
| | PUL 26 | mannosidase | | | | |
| Bova115_1154 | Predicted | alpha-mannosidase | GH38 | 98 | 0.02 | 9.0E−01 |
| | PUL 26 | | | | | |
| Bova115_1155 | Predicted | hypothetical protein | | 97 | 0.00 | 1.0E+00 |
| | PUL 26 | | | | | |
| Bova115_1156 | Predicted | RNA polymerase | REG | 99 | 0.01 | 1.0E+00 |
| | PUL 26 | ECF-type sigma factor | | | | |
| Bova115_1157 | Predicted | Putative regulatory | REG | 99 | 0.13 | 3.9E−01 |
| | PUL 26 | protein | | | | |
| Bova115_1353 | Predicted | RNA polymerase | REG | 99 | 0.01 | 1.0E+00 |
| | PUL 28 | ECF-type sigma factor | | | | |
| Bova115_1354 | Predicted | Putative anti-sigma | REG | 100 | 0.08 | 7.4E−01 |
| | PUL 28 | factor | | | | |
| Bova115_1355 | Predicted | Alpha-1,2- | GH92 | 99 | 0.02 | 9.4E−01 |
| | PUL 28 | mannosidase | | | | |
| Bova115_1356 | Predicted | Alpha-1,2- | GH92 | 99 | −0.06 | 1.0E+00 |
| | PUL 28 | mannosidase | | | | |
| Bova115_1361 | Predicted | Outer membrane | Trans | 69 | 0.04 | 8.8E−01 |
| | PUL 28 | TonB-dependent | | | | |
| | | transporter, SusC | | | | |
| | | family | | | | |
| Bova115_1362 | Predicted | Cell surface glycan- | SusD | 43 | −0.02 | 1.0E+00 |
| | PUL 28 | binding lipoprotein, | | | | |
| | | SusD family | | | | |
| Bova115_1360 | Predicted | hypothetical protein | GH | 32 | −0.01 | 1.0E+00 |
| | PUL 28 | | | | | |
| Bova115_1364 | Predicted | putative patatin-like | | 40 | 0.01 | 9.7E−01 |
| | PUL 28 | protein | | | | |
| Bova115_1365 | Predicted | hypothetical protein | GH18 | 30 | 0.00 | 1.0E+00 |
| | PUL 28 | | | | | |
| Bova115_1385 | Predicted | Alpha-glucosidase | GH97 | 97 | −0.08 | 7.3E−01 |
| | PUL 29 | (EC 3.2.1.20) | | | | |
| Bova115_1386 | Predicted | Alpha-1,2- | GH92 | 98 | −0.02 | 1.0E+00 |
| | PUL 29 | mannosidase | | | | |
| Bova115_1387 | Predicted | putative secretory | | 87 | −0.01 | 1.0E+00 |
| | PUL 29 | protein | | | | |
| Bova115 1388 | Predicted | Alpha-1,2- | GH92 | 91 | −0.01 | 1.0E+00 |
| | PUL 29 | mannosidase | | | | |
| Bova115_1389 | Predicted | Alpha-1,2- | GH92 | 93 | −0.02 | 1.0E+00 |
| | PUL 29 | mannosidase | | | | |
| Bova115_1392 | Predicted | DNA-binding response | REG | 67 | 0.06 | 8.4E−01 |
| | PUL 29 | regulator, AraC family | | | | |
| Bova115_1402 | Predicted | Cell surface glycan- | SusD | 99 | −1.07 | 4.1E−03 |
| | PUL 30 | binding lipoprotein, | | | | |
| | | SusD family | | | | |
| Bova115_1403 | Predicted | Outer membrane | Trans | 99 | −0.11 | 5.8E−01 |
| | PUL 30 | TonB-dependent | | | | |
| | | transporter, SusC | | | | |
| | | family | | | | |
| Bova115_1429 | | hypothetical protein | | 93 | 0.01 | 1.0E+00 |
| Bova115_3454 | Predicted | Cell surface glycan- | SusD | 61 | −0.02 | 1.0E+00 |
| | PUL 86 | binding lipoprotein, | | | | |
| | | SusD family | | | | |
| Bova115_1487 | Predicted | Cell surface glycan- | SusD | 99 | −0.02 | 1.0E+00 |
| | PUL 31 | binding lipoprotein, | | | | |
| | | SusD family | | | | |
| Bova115_1488 | Predicted | Outer membrane | Trans | 98 | −0.01 | 1.0E+00 |
| | PUL 31 | TonB-dependent | | | | |
| | | transporter, SusC | | | | |
| | | family | | | | |
| Bova115_1489 | Predicted | Oligo alginate lyase | PL15 | 95 | 0.04 | 1.0E+00 |
| | PUL 31 | | | | | |
| Bova115_1556 | Predicted | hypothetical protein | | 100 | 0.03 | 9.0E−01 |
| | PUL 32 | | | | | |
| Bova115_1557 | Predicted | hypothetical protein | | 100 | −0.02 | 1.0E+00 |
| | PUL 32 | | | | | |
| Bova115_1558 | Predicted | Probable zinc | Pept | 100 | −0.02 | 1.0E+00 |
| | PUL 32 | protease pqqL (EC | | | | |
| | | 3.4.99.—) | | | | |
| Bova115_1559 | Predicted | hypothetical protein | | 100 | 0.00 | 1.0E+00 |
| | PUL 32 | | | | | |
| Bova115_1560 | Predicted | Cell surface glycan- | SusD | 100 | 0.03 | 1.0E+00 |
| | PUL 32 | binding lipoprotein, | | | | |
| | | SusD family | | | | |
| Bova115_1561 | Predicted | Outer membrane | Trans | 100 | −0.10 | 6.5E−01 |
| | PUL 32 | TonB-dependent | | | | |
| | | transporter, SusC | | | | |
| | | family | | | | |
| Bova115_1562 | Predicted | Putative anti-sigma | REG | 100 | 0.39 | 1.1E−01 |
| | PUL 32 | factor | | | | |
| Bova115_1563 | Predicted | RNA polymerase | REG | 100 | 0.02 | 1.0E+00 |
| | PUL 32 | ECF-type sigma factor | | | | |
| Bova115_1571 | Predicted | hypothetical protein | | 99 | −0.04 | 1.0E+00 |
| | PUL 33 | | | | | |
| Bova115_1572 | Predicted | Cell surface glycan- | SusD | 100 | −0.02 | 1.0E+00 |
| | PUL 33 | binding lipoprotein, | | | | |
| | | SusD family | | | | |
| Bova115_1573 | Predicted | Outer membrane | Trans | 100 | −0.01 | 1.0E+00 |
| | PUL 33 | TonB-dependent | | | | |
| | | transporter, SusC | | | | |
| | | family | | | | |
| Bova115_1574 | Predicted | hydrolase (secreted | GH76 | 100 | 0.03 | 1.0E+00 |
| | PUL 33 | protein) | | | | |
| Bova115_1575 | Predicted | hypothetical protein | | 100 | 0.00 | 1.0E+00 |
| | PUL 33 | | | | | |
| Bova115_1576 | Predicted | hypothetical protein | | 99 | 0.03 | 1.0E+00 |
| | PUL 33 | | | | | |
| Bova115_1577 | Predicted | DNA-binding response | REG | 98 | 0.01 | 1.0E+00 |
| | PUL 33 | regulator, AraC family | | | | |
| Bova115_1578 | Predicted | Alpha-1,2- | GH92 | 98 | −0.03 | 1.0E+00 |
| | PUL 33 | mannosidase | | | | |
| Bova115_1579 | Predicted | Bacillopeptidase F | | 91 | 0.07 | 1.0E+00 |
| | PUL 33 | precursor (EC 3.4.21.—) | | | | |
| Bova115_1580 | Predicted | Putative hydrolase | GH76 | 96 | −0.12 | 5.6E−01 |
| | PUL 33 | | | | | |
| Bova115_1581 | Predicted | hypothetical protein | GH125 | 100 | −0.02 | 1.0E+00 |
| | PUL 33 | | | | | |
| Bova115_1582 | Predicted | FIG00418227: | | 92 | 0.01 | 1.0E+00 |
| | PUL 33 | hypothetical protein | | | | |
| Bova115_1583 | Predicted | FIG01423360: | GH130 | 100 | 0.01 | 1.0E+00 |
| | PUL 33 | glycoside hydrolase | | | | |
| Bova115_1584 | Predicted | Alpha-1,2- | GH92 | 99 | −0.04 | 8.8E−01 |
| | PUL 33 | mannosidase | | | | |
| Bova115_1607 | Predicted | acetyl xylan esterase | CE6 | 99 | 0.02 | 9.1E−01 |
| | PUL 34 | A | | | | |
| Bova115_1608 | Predicted | beta-galactosidase | GH2- | 100 | −0.06 | 8.1E−01 |
| | PUL 34 | (EC 3.2.1.23) | CBM57 | | | |
| Bova115_1609 | Predicted | alpha-xylosidase (EC | GH31 | 99 | 0.03 | 1.0E+00 |
| | PUL 34 | 3.2.1.177) | | | | |
| Bova115_1610 | Predicted | hypothetical protein | GH | 99 | 0.04 | 1.0E+00 |
| | PUL 34 | | | | | |
| Bova115_1611 | Predicted | hypothetical protein | | 100 | −0.07 | 7.4E−01 |
| | PUL 34 | | | | | |
| Bova115_1612 | Predicted | hypothetical protein | GH50 | 99 | 0.01 | 1.0E+00 |
| | PUL 34 | | | | | |
| Bova115_1613 | Predicted | hypothetical protein | | 63 | 0.01 | 1.0E+00 |
| | PUL 34 | | | | | |
| Bova115_1614 | Predicted | hypothetical protein | | 100 | −0.01 | 1.0E+00 |
| | PUL 34 | | | | | |
| Bova115_1615 | Predicted | Cell surface glycan- | SusD | 100 | 0.01 | 1.0E+00 |
| | PUL 34 | binding lipoprotein, | | | | |
| | | SusD family | | | | |
| Bova115_1616 | Predicted | Outer membrane | Trans | 100 | −0.03 | 1.0E+00 |
| | PUL 34 | TonB-dependent | | | | |
| | | transporter, SusC | | | | |
| | | family | | | | |
| Bova115_1618 | Predicted | BaeS-type histidine | REG | 99 | 0.00 | 1.0E+00 |
| | PUL 34 | kinase/OmpR-type | | | | |
| | | DNA-binding response | | | | |
| | | regulator | | | | |
| Bova115_1625 | Predicted | hypothetical protein | | 100 | 0.04 | 8.6E−01 |
| | PUL 35 | | | | | |
| Bova115_1626 | Predicted | hypothetical protein | | 100 | 0.05 | 1.0E+00 |
| | PUL 35 | | | | | |
| Bova115_1627 | Predicted | Cell surface glycan- | SusD | 100 | 0.03 | 1.0E+00 |
| | PUL 35 | binding lipoprotein, | | | | |
| | | SusD family | | | | |
| Bova115_1628 | Predicted | Outer membrane | Trans | 100 | −0.05 | 8.3E−01 |
| | PUL 35 | TonB-dependent | | | | |
| | | transporter, SusC | | | | |
| | | family | | | | |
| Bova115_1629 | Predicted | putative anti-sigma | REG | 99 | 0.09 | 6.9E−01 |
| | PUL 35 | factor | | | | |
| Bova115_1630 | Predicted | RNA polymerase | REG | 100 | 0.10 | 6.5E−01 |
| | PUL 35 | ECF-type sigma factor | | | | |
| Bova115_1652 | Predicted | hypothetical protein | | 99 | −0.01 | 1.0E+00 |
| | PUL 36 | | | | | |
| Bova115_1653 | Predicted | Endo-beta-N- | GH18 | 99 | 0.04 | 1.0E+00 |
| | PUL 36 | acetylglucosaminidase | | | | |
| | | F2 | | | | |
| Bova115_1654 | Predicted | Cell surface glycan- | SusD | 99 | 0.02 | 9.3E−01 |
| | PUL 36 | binding lipoprotein, | | | | |
| | | SusD family | | | | |
| Bova115_1655 | Predicted | Outer membrane | Trans | 99 | −0.12 | 5.5E−01 |
| | PUL 36 | TonB-dependent | | | | |
| | | transporter, SusC | | | | |
| | | family | | | | |
| Bova115_1656 | Predicted | Putative anti-sigma | REG | 98 | 0.05 | 1.0E+00 |
| | PUL 36 | factor | | | | |
| Bova115_1657 | Predicted | RNA polymerase | REG | 96 | 0.02 | 1.0E+00 |
| | PUL 36 | ECF-type sigma factor | | | | |
| Bova115_1686 | Predicted | Regulatory protein | REG | 83 | −0.06 | 1.0E+00 |
| | PUL 37 | SusR | | | | |
| Bova115_1687 | Predicted | Alpha-amylase | GH13 | 82 | −0.07 | 6.9E−01 |
| | PUL 37 | (Neopullulanase) | | | | |
| | | SusA (EC 3.2.1.135) | | | | |
| Bova115_1688 | Predicted | Glucan 1,4-alpha- | GH97 | 92 | 0.01 | 1.0E+00 |
| | PUL 37 | glucosidase (EC | | | | |
| | | 3.2.1.3) | | | | |
| Bova115_1689 | Predicted | Outer membrane | Trans | 63 | 0.00 | 1.0E+00 |
| | PUL 37 | TonB-dependent | | | | |
| | | transporter, SusC | | | | |
| | | family | | | | |
| Bova115_1690 | Predicted | Cell surface glycan- | SusD | 47 | 0.03 | 1.0E+00 |
| | PUL 37 | binding lipoprotein, | | | | |
| | | SusD family | | | | |
| Bova115_1691 | Predicted | outer membrane | | 36 | 0.00 | 1.0E+00 |
| | PUL 37 | protein SusE | | | | |
| Bova115_1692 | Predicted | hypothetical protein | | 27 | 0.02 | 1.0E+00 |
| | PUL 37 | | | | | |
| Bova115_1693 | Predicted | 1,4-alpha-glucan | GH13- | 43 | −0.19 | 1.1E−01 |
| | PUL 37 | branching enzyme | CBM48 | | | |
| | | (EC 2.4.1.18) | | | | |
| Bova115_1694 | | UDP-2,3- | | 96 | −0.07 | 7.3E−01 |
| | | diacylglucosamine | | | | |
| | | diphosphatase (EC | | | | |
| | | 3.6.1.54) | | | | |
| Bova115_1695 | | PaaD-like protein | | 95 | 0.08 | 7.0E−01 |
| | | (DUF59) involved in | | | | |
| | | Fe—S cluster assembly | | | | |
| Bova115_2258 | Predicted | hypothetical protein | GH | 33 | −0.09 | 5.9E−01 |
| | PUL 52 | | | | | |
| Bova115_2259 | Predicted | Cell surface glycan- | SusD | 58 | 0.01 | 1.0E+00 |
| | PUL 52 | binding lipoprotein, | | | | |
| | | SusD family | | | | |
| Bova115_4029 | Predicted | Outer membrane | Trans | 71 | 0.07 | 6.9E−01 |
| | PUL 95 | TonB-dependent | | | | |
| | | transporter, SusC | | | | |
| | | family | | | | |
| Bova115_1696 | | UPF0758 family | | 95 | −0.02 | 9.2E−01 |
| | | protein | | | | |
| Bova115_1505 | | beta-glycosyl | GH20 | 45 | 0.01 | 9.9E−01 |
| | | hydrolase | | | | |
| Bova115_1707 | CAZyme | hypothetical protein | GH145 | 92 | −0.03 | 1.0E+00 |
| | clucter 4 | | | | | |
| Bova115_1708 | CAZyme | beta-galactosidase | GH43_24 | 95 | 0.03 | 1.0E+00 |
| | clucter 4 | (EC 3.2.1.23) | | | | |
| Bova115_1710 | CAZyme | hypothetical protein | GH154 | 90 | 0.03 | 1.0E+00 |
| | clucter 4 | | | | | |
| Bova115_1243 | Predicted | alpha-xylosidase (EC | GH31 | 80 | −0.02 | 1.0E+00 |
| | PUL 27 | 3.2.1.177) | | | | |
| Bova115_1716 | CAZyme | Xylan 1,4-beta- | GH97 | 94 | 0.00 | 1.0E+00 |
| | cluster 5 | xylosidase (EC | | | | |
| | | 3.2.1.37) | | | | |
| Bova115_1717 | CAZyme | alpha-L- | GH51 | 95 | 0.01 | 1.0E+00 |
| | cluster 5 | arabinofuranosidase | | | | |
| | | (EC 3.2.1.55) | | | | |
| Bova115_1718 | CAZyme | Hypothetical glycoside | GH43_19 | 94 | 0.06 | 8.3E−01 |
| | cluster 5 | hydrolase, family 43, | | | | |
| | | similar to | | | | |
| | | arabinosidase | | | | |
| Bova115_1719 | CAZyme | beta-galactosidase | CBM32- | 95 | 0.01 | 1.0E+00 |
| | cluster 5 | (EC 3.2.1.23) | GH35 | | | |
| Bova115_1749 | | hypothetical protein | GH115 | 57 | −0.01 | 9.7E−01 |
| Bova115_1754 | Predicated | Putative large | GH95 | 92 | −0.01 | 1.0E+00 |
| | PUL 38 | secreted protein | | | | |
| | | SCO0341 | | | | |
| Bova115_1755 | Predicted | BaeS-type histidine | REG | 91 | −0.03 | 8.9E−01 |
| | PUL 38 | kinase/OmpR-type | | | | |
| | | DNA-binding response | | | | |
| | | regulator | | | | |
| Bova115_1758 | Predicted | Endo-1,4-beta- | CBM6- | 79 | −0.01 | 1.0E+00 |
| | PUL 38 | xylanase (EC 3.2.1.8) | GH43_29 | | | |
| Bova115_1756 | Predicted | Glycoprotein gp2 | CE6 | 67 | 0.04 | 1.0E+00 |
| | PUL 38 | | | | | |
| Bova115_1761 | Predicted | hypothetical protein | GH5_21 | 54 | 0.02 | 1.0E+00 |
| | PUL 38 | | | | | |
| Bova115_1762 | Predicted | hypothetical protein | | 49 | 0.00 | 1.0E+00 |
| | PUL 38 | | | | | |
| Bova115_1763 | Predicted | Cell surface glycan- | SusD | 53 | −0.01 | 1.0E+00 |
| | PUL 38 | binding lipoprotein, | | | | |
| | | SusD family | | | | |
| Bova115_1764 | Predicted | Outer membrane | Trans | 53 | 0.03 | 9.0E−01 |
| | PUL 38 | TonB-dependent | | | | |
| | | transporter, SusC | | | | |
| | | family | | | | |
| Bova115_1765 | Predicted | Cell surface glycan- | SusD | 48 | 0.04 | 9.0E−01 |
| | PUL 38 | binding lipoprotein, | | | | |
| | | SusD family | | | | |
| Bova115_1766 | Predicted | Outer membrane | Trans | 63 | −0.87 | 1.0E+00 |
| | PUL 38 | TonB-dependent | | | | |
| | | transporter, SusC | | | | |
| | | family | | | | |
| Bova115_1767 | Predicted | Xylan 1,4-beta- | GH43_12 | 87 | −0.01 | 1.0E+00 |
| | PUL 38 | xylosidase (EC | | | | |
| | | 3.2.1.37) | | | | |
| Bova115_1769 | Predicted | Hypothetical glycoside | CBM6- | 91 | 0.02 | 1.0E+00 |
| | PUL 38 | hydrolase, family 43, | GH43_29 | | | |
| | | similar to | | | | |
| | | arabinosidase | | | | |
| Bova115_1771 | Predicted | Xylan 1,4-beta- | GH97 | 97 | −0.03 | 1.0E+00 |
| | PUL 38 | xylosidase (EC | | | | |
| | | 3.2.1.37) | | | | |
| Bova115_1772 | Predicted | alpha-xylosidase (EC | GH31 | 97 | 0.01 | 1.0E+00 |
| | PUL 38 | 3.2.1.177) | | | | |
| Bova115_1773 | Predicted | Xylan 1,4-beta- | GH43_12 | 98 | −0.02 | 1.0E+00 |
| | PUL 38 | xylosidase (EC | | | | |
| | | 3.2.1.37) | | | | |
| Bova115_1775 | Predicted | beta-glucosidase (EC | GH3 | 98 | 0.06 | 7.3E−01 |
| | PUL 38 | 3.2.1.21) | | | | |
| Bova115_1776 | Predicted | Endo-1,4-beta- | GH43_10 | 99 | 0.00 | 1.0E+00 |
| | PUL 38 | xylanase (EC 3.2.1.8) | | | | |
| Bova115_1794 | Predicted | N-acetyl glucosamine | Trans | 89 | 0.01 | 1.0E+00 |
| | PUL 39 | transporter, NagP | | | | |
| Bova115_1795 | Predicted | N-acetylglucosamine- | CE9 | 82 | 0.02 | 1.0E+00 |
| | PUL 39 | 6-phosphate | | | | |
| | | deacetylase (EC | | | | |
| | | 3.5.1.25) | | | | |
| Bova115_1796 | Predicted | Glucosamine-6- | | 83 | 0.01 | 1.0E+00 |
| | PUL 39 | phosphate deaminase | | | | |
| | | (EC 3.5.99.6) | | | | |
| Bova115_1797 | Predicted | Putative | | 79 | 0.03 | 1.0E+00 |
| | PUL 39 | dehydrogenase | | | | |
| Bova115_1798 | Predicted | Putative | | 82 | −0.02 | 1.0E+00 |
| | PUL 39 | oxidoreductase | | | | |
| Bova115_1799 | Predicted | thiamine biosynthesis | | 80 | −0.01 | 1.0E+00 |
| | PUL 39 | lipoprotein ApbE | | | | |
| Bova115_1800 | Predicted | putative hydrogenase | | 96 | −0.01 | 1.0E+00 |
| | PUL 39 | | | | | |
| Bova115_1801 | Predicted | hypothetical protein | | 99 | — | — |
| | PUL 39 | | | | | |
| Bova115_1802 | Predicted | Myo-inositol 2- | | 100 | −0.12 | 1.0E+00 |
| | PUL 39 | dehydrogenase (EC | | | | |
| | | 1.1.1.18) | | | | |
| Bova115_1813 | | DNA topoisomerase | | 97 | 0.01 | 9.9E−01 |
| | | IV subunit A (EC | | | | |
| | | 5.99.1.3) | | | | |
| Bova115_1814 | | hypothetical protein | | 98 | 0.01 | 1.0E+00 |
| Bova115_1815 | | Carboxyl-terminal | Pept | 82 | −0.02 | 1.0E+00 |
| | | protease-related | | | | |
| | | protein | | | | |
| Bova115_1817 | Predicted | Outer membrane | Trans | 99 | 0.34 | 2.0E−03 |
| | PUL 41 | TonB-dependent | | | | |
| | | transporter, SusC | | | | |
| | | family | | | | |
| Bova115_1818 | Predicted | Cell surface glycan- | SusD | 97 | −0.39 | 1.2E−04 |
| | PUL 41 | binding lipoprotein, | | | | |
| | | SusD family | | | | |
| Bova115_1819 | Predicted | beta-glucosidase (EC | GH3 | 100 | −0.13 | 5.0E−01 |
| | PUL 41 | 3.2.1.21) | | | | |
| Bova115_1820 | Predicted | Periplasmic beta- | GH144 | 99 | −0.58 | 5.7E−05 |
| | PUL 41 | glucosidase (EC | | | | |
| | | 3.2.1.21) | | | | |
| Bova115_1859 | Predicted | hypothetical protein | GH125 | 99 | −0.03 | 1.0E+00 |
| | PUL 42 | | | | | |
| Bova115_1860 | Predicted | Alpha-1,2- | GH92 | 99 | −0.02 | 1.0E+00 |
| | PUL 42 | mannosidase | | | | |
| Bova115_1861 | Predicted | Glutaminase A | | 99 | 0.01 | 1.0E+00 |
| | PUL 42 | | | | | |
| Bova115_1862 | Predicted | hypothetical protein | | 99 | 0.04 | 1.0E+00 |
| | PUL 42 | | | | | |
| Bova115_1863 | Predicted | alpha-1,6-mannanase | GH76 | 97 | −0.01 | 9.8E−01 |
| | PUL 42 | | | | | |
| Bova115_1864 | Predicted | hypothetical protein | | 96 | 0.04 | 1.0E+00 |
| | PUL 42 | | | | | |
| Bova115_1865 | Predicted | hypothetical protein | | 100 | 0.01 | 1.0E+00 |
| | PUL 42 | | | | | |
| Bova115_1866 | Predicted | alpha-1,6-mannanase | GH76 | 99 | 0.00 | 1.0E+00 |
| | PUL 42 | | | | | |
| Bova115_1867 | Predicted | Cell surface glycan- | SusD | 100 | −0.01 | 1.0E+00 |
| | PUL 42 | binding lipoprotein, | | | | |
| | | SusD family | | | | |
| Bova115_1868 | Predicted | Outer membrane | Trans | 100 | −0.01 | 1.0E+00 |
| | PUL 42 | TonB-dependent | | | | |
| | | transporter, SusC | | | | |
| | | family | | | | |
| Bova115_1869 | Predicted | Putative anti-sigma | REG | 99 | −0.01 | 9.9E−01 |
| | PUL 42 | factor | | | | |
| Bova115_1870 | Predicted | RNA polymerase | REG | 98 | −0.03 | 1.0E+00 |
| | PUL 42 | ECF-type sigma factor | | | | |
| Bova115_1871 | CAZyme | arabinan endo-1,5- | GH43_34 | 99 | −0.04 | 8.8E−01 |
| | cluster 6 | alpha-L-arabinosidase | | | | |
| | | A precursor | | | | |
| Bova115 1872 | CAZyme | alpha-L- | GH43_26 | 96 | 0.02 | 1.0E+00 |
| | cluster 6 | arabinofuranosidase II | | | | |
| | | (EC 3.2.1.55) | | | | |
| Bova115_1874 | CAZyme | beta-galactosidase | GH2 | 99 | −0.03 | 1.0E+00 |
| | cluster 6 | (EC 3.2.1.23) | | | | |
| Bova115_1875 | CAZyme | Arylsulfatase (EC | Sulf | 99 | −0.04 | 1.0E+00 |
| | cluster 6 | 3.1.6.1) | | | | |
| Bova115_1878 | | Integrase | Int | 100 | 0.01 | 9.9E−01 |
| Bova115_1879 | | Glutaminase A | | 98 | −0.02 | 1.0E+00 |
| Bova115_1885 | | hypothetical protein | | 99 | 0.01 | 1.0E+00 |
| Bova115_1886 | | Arylsulfatase (EC | Sulf | 100 | −0.01 | 1.0E+00 |
| | | 3.1.6.1) | | | | |
| Bova115_1887 | | Arylsulfatase (EC | Sulf | 100 | 0.00 | 1.0E+00 |
| | | 3.1.6.1) | | | | |
| Bova115_1888 | | BaeS-type histidine | REG | 96 | 0.03 | 1.0E+00 |
| | | kinase/OmpR-type | | | | |
| | | DNA-binding response | | | | |
| | | regulator | | | | |
| Bova115_2008 | Predicted | Chondroitinase | PL8_2 | 99 | −0.01 | 1.0E+00 |
| | PUL 44 | (chondroitin lyase) | | | | |
| Bova115_2009 | Predicted | N- | Sulf | 100 | −0.01 | 1.0E+00 |
| | PUL 44 | acetylgalactosamine | | | | |
| | | 6-sulfate sulfatase | | | | |
| | | (GALNS) | | | | |
| Bova115_2010 | Predicted | Glucuronyl hydrolase | GH88 | 99 | 0.01 | 1.0E+00 |
| | PUL 44 | | | | | |
| Bova115_2012 | Predicted | beta-galactosidase | GH2 | 100 | −0.03 | 1.0E+00 |
| | PUL 44 | (EC 3.2.1.23) | | | | |
| Bova115_2013 | Predicted | hypothetical protein | | 99 | 0.01 | 1.0E+00 |
| | PUL 44 | | | | | |
| Bova115_2014 | Predicted | Arabinan endo-1,5- | GH43_3 | 99 | 0.02 | 1.0E+00 |
| | PUL 44 | alpha-L-arabinosidase | | | | |
| | | (EC 3.2.1.99) | | | | |
| Bova115_2015 | Predicted | Cell surface glycan- | SusD | 99 | 0.03 | 1.0E+00 |
| | PUL 44 | binding lipoprotein, | | | | |
| | | SusD family | | | | |
| Bova115_2016 | Predicted | Outer membrane | Trans | 99 | −0.04 | 8.6E−01 |
| | PUL 44 | TonB-dependent | | | | |
| | | transporter, SusC | | | | |
| | | family | | | | |
| Bova115_2017 | Predicted | hypothetical protein | | 99 | 0.00 | 1.0E+00 |
| | PUL 44 | | | | | |
| Bova115_2019 | Predicted | RNA polymerase | REG | 99 | −0.06 | 7.5E−01 |
| | PUL 44 | ECF-type sigma factor | | | | |
| Bova115_2020 | Predicted | Putative anti-sigma | REG | 99 | −0.20 | 3.1E−01 |
| | PUL 44 | factor | | | | |
| Bova115_2054 | Predicted | BaeS-type histidine | REG | 100 | −0.02 | 9.6E−01 |
| | PUL 46 | kinase/OmpR-type | | | | |
| | | DNA-binding response | | | | |
| | | regulator | | | | |
| Bova115_2055 | Predicted | Arylsulfatase (EC | Sulf | 99 | 0.05 | 1.0E+00 |
| | PUL 46 | 3.1.6.1) | | | | |
| Bova115_2056 | Predicted | hypothetical protein | | 100 | — | — |
| | PUL 46 | | | | | |
| Bova115_2057 | Predicted | Outer membrane | Trans | 100 | −0.02 | 1.0E+00 |
| | PUL 46 | TonB-dependent | | | | |
| | | transporter, SusC | | | | |
| | | family | | | | |
| Bova115_2058 | Predicted | Cell surface glycan- | SusD | 100 | −0.01 | 9.8E−01 |
| | PUL 46 | binding lipoprotein, | | | | |
| | | SusD family | | | | |
| Bova115_2059 | Predicted | hypothetical protein | | 95 | −0.05 | 1.0E+00 |
| | PUL 46 | | | | | |
| Bova115_2060 | Predicted | tRNA-dihydrouridine | | 97 | 0.00 | 1.0E+00 |
| | PUL 46 | synthase BT3326 | | | | |
| Bova115_2061 | Predicted | hypothetical protein | | 93 | 0.01 | 1.0E+00 |
| | PUL 46 | | | | | |
| Bova115_2062 | Predicted | Chondroitinase | PL8_2 | 99 | 0.05 | 1.0E+00 |
| | PUL 46 | (chondroitin lyase) | | | | |
| Bova115_2063 | Predicted | hypothetical protein | | 99 | 0.00 | 1.0E+00 |
| | PUL 46 | | | | | |
| Bova115_2090 | Predicted | beta-glucosidase (EC | GH3 | 98 | −0.25 | 2.6E−02 |
| | PUL 47 | 3.2.1.21) | | | | |
| Bova115_2092 | Predicted | hypothetical protein | | 49 | 0.05 | 1.0E+00 |
| | PUL 47 | | | | | |
| Bova115_2093 | Predicted | glycoside hyrdolase | GH30_3 | 94 | −0.03 | 1.0E+00 |
| | PUL 47 | family 30, candidate | | | | |
| | | beta-glycosidase | | | | |
| Bova115_2094 | Predicted | Cell surface glycan- | SusD | 94 | −0.05 | 1.0E+00 |
| | PUL 47 | binding lipoprotein, | | | | |
| | | SusD family | | | | |
| Bova115_2095 | Predicted | Outer membrane | Trans | 97 | −0.05 | 1.0E+00 |
| | PUL 47 | TonB-dependent | | | | |
| | | transporter, SusC | | | | |
| | | family | | | | |
| Bova115_2096 | Predicted | Regulatory protein | REG | 92 | 0.04 | 1.0E+00 |
| | PUL 47 | SusR | | | | |
| Bova115_2115 | Predicted | RNA polymerase | REG | 100 | −0.01 | 1.0E+00 |
| | PUL 49 | ECF-type sigma factor | | | | |
| Bova115_2116 | Predicted | putative anti-sigma | REG | 100 | −0.01 | 1.0E+00 |
| | PUL 49 | factor | | | | |
| Bova115_2117 | Predicted | Outer membrane | Trans | 100 | 0.01 | 1.0E+00 |
| | PUL 49 | TonB-dependent | | | | |
| | | transporter, SusC | | | | |
| | | family | | | | |
| Bova115_2118 | Predicted | Cell surface glycan- | SusD | 100 | 0.00 | 1.0E+00 |
| | PUL 49 | binding lipoprotein, | | | | |
| | | SusD family | | | | |
| Bova115_2119 | Predicted | hypothetical protein | | 100 | −0.04 | 1.0E+00 |
| | PUL 49 | | | | | |
| Bova115_2120 | Predicted | Glycerophosphoryl | | 99 | 0.01 | 1.0E+00 |
| | PUL 49 | diester | | | | |
| | | phosphodiesterase | | | | |
| | | (EC 3.1.4.46) | | | | |
| Bova115_2121 | Predicted | Glycerophosphoryl | | 100 | −0.03 | 1.0E+00 |
| | PUL 49 | diester | | | | |
| | | phosphodiesterase | | | | |
| | | (EC 3.1.4.46) | | | | |
| Bova115_2128 | | Regulatory protein | REG | 78 | 0.05 | 1.0E+00 |
| | | SusR | | | | |
| Bova115_2129 | | Putative alpha-1,6- | GH76 | 93 | 0.00 | 1.0E+00 |
| | | mannanase | | | | |
| Bova115_2130 | | hypothetical protein | GH125 | 95 | −0.02 | 1.0E+00 |
| Bova115_2143 | Predicted | hypothetical protein | | 100 | −0.04 | 1.0E+00 |
| | PUL 50 | | | | | |
| Bova115_2144 | Predicted | hypothetical protein | Pept | 100 | 0.05 | 1.0E+00 |
| | PUL 50 | | | | | |
| Bova115_2145 | Predicted | Dipeptidyl peptidase | Pept | 99 | −0.03 | 1.0E+00 |
| | PUL 50 | IV | | | | |
| Bova115_2146 | Predicted | hypothetical protein | Pept | 100 | 0.02 | 1.0E+00 |
| | PUL 50 | | | | | |
| Bova115_2147 | Predicted | hypothetical protein | | 100 | 0.00 | 1.0E+00 |
| | PUL 50 | | | | | |
| Bova115_2148 | Predicted | hypothetical protein | | 100 | 0.03 | 1.0E+00 |
| | PUL 50 | | | | | |
| Bova115_2149 | Predicted | Cell surface glycan- | SusD | 100 | 0.00 | 1.0E+00 |
| | PUL 50 | binding lipoprotein, | | | | |
| | | SusD family | | | | |
| Bova115_2150 | Predicted | Outer membrane | Trans | 100 | −0.02 | 9.4E−01 |
| | PUL 50 | TonB-dependent | | | | |
| | | transporter, SusC | | | | |
| | | family | | | | |
| Bova115_2151 | Predicted | Putative anti-sigma | REG | 100 | 0.12 | 1.0E+00 |
| | PUL 50 | factor | | | | |
| Bova115_2152 | Predicted | RNA polymerase | REG | 100 | −0.01 | 1.0E+00 |
| | PUL 50 | sigma-70 factor | | | | |
| Bova115_2192 | Predicted | Outer membrane | Trans | 100 | 0.04 | 1.0E+00 |
| | PUL 51 | TonB-dependent | | | | |
| | | transporter, SusC | | | | |
| | | family | | | | |
| Bova115_2193 | Predicted | Cell surface glycan- | SusD | 100 | −0.01 | 1.0E+00 |
| | PUL 51 | binding lipoprotein, | | | | |
| | | SusD family | | | | |
| Bova115_2194 | Predicted | Two-component | | 100 | 0.02 | 1.0E+00 |
| | PUL 51 | system sensor | | | | |
| | | histidine kinase | | | | |
| Bova115_2243 | Predicted | FIG01423360: | GH130 | 100 | 0.03 | 1.0E+00 |
| | PUL 52 | glycoside hydrolase | | | | |
| Bova115_2244 | Predicted | hypothetical protein | Trans | 100 | 0.00 | 1.0E+00 |
| | PUL 52 | | | | | |
| Bova115_2245 | Predicted | hypothetical protein | | 99 | 0.00 | 1.0E+00 |
| | PUL 52 | | | | | |
| Bova115_2246 | Predicted | Outer membrane | Trans | 100 | 0.07 | 1.0E+00 |
| | PUL 52 | TonB-dependent | | | | |
| | | transporter, SusC | | | | |
| | | family | | | | |
| Bova115_2247 | Predicted | Cell surface glycan- | SusD | 100 | 0.00 | 1.0E+00 |
| | PUL 52 | binding lipoprotein, | | | | |
| | | SusD family | | | | |
| Bova115_2248 | Predicted | hypothetical protein | | 98 | 0.01 | 1.0E+00 |
| | PUL 52 | | | | | |
| Bova115_2249 | Predicted | hypothetical protein | REG | 99 | −0.04 | 9.0E−01 |
| | PUL 52 | | | | | |
| Bova115_2275 | Predicted | hypothetical protein | | 91 | 0.00 | 1.0E+00 |
| | PUL 53 | | | | | |
| Bova115_2276 | Predicted | UDP-glucose 4- | | 97 | −0.01 | 1.0E+00 |
| | PUL 53 | epimerase (EC | | | | |
| | | 5.1.3.2) | | | | |
| Bova115_2277 | Predicted | Glucuronyl hydrolase | GH88 | 95 | 0.00 | 1.0E+00 |
| | PUL 53 | | | | | |
| Bova115_2278 | Predicted | hypothetical protein | PL35 | 98 | 0.05 | 1.0E+00 |
| | PUL 53 | | | | | |
| Bova115_2279 | Predicted | hypothetical protein | PL35 | 96 | 0.04 | 1.0E+00 |
| | PUL 53 | | | | | |
| Bova115_2280 | Predicted | Cell surface glycan- | SusD | 97 | −0.02 | 1.0E+00 |
| | PUL 53 | binding lipoprotein, | | | | |
| | | SusD family | | | | |
| Bova115_2281 | Predicted | Outer membrane | Trans | 99 | 0.04 | 8.8E−01 |
| | PUL 53 | TonB-dependent | | | | |
| | | transporter, SusC | | | | |
| | | family | | | | |
| Bova115_2282 | Predicted | sialic acid-specific 9- | | 87 | 0.03 | 1.0E+00 |
| | PUL 53 | O-acetylesterase | | | | |
| Bova115_2283 | Predicted | BaeS-type histidine | REG | 98 | 0.00 | 1.0E+00 |
| | PUL 53 | kinase/OmpR-type | | | | |
| | | DNA-binding response | | | | |
| | | regulator | | | | |
| Bova115_2284 | Predicted | Radical SAM protein | | 100 | 0.02 | 9.3E−01 |
| | PUL 53 | BT_3168 | | | | |
| Bova115_2285 | Predicted | hypothetical protein | | 98 | 0.00 | 1.0E+00 |
| | PUL 53 | | | | | |
| Bova115_2286 | Predicted | hypothetical protein | | 90 | −0.01 | 1.0E+00 |
| | PUL 53 | | | | | |
| Bova115_2287 | Predicted | hypothetical protein | | 63 | 0.03 | 1.0E+00 |
| | PUL 53 | | | | | |
| Bova115_2288 | Predicted | hypothetical protein | | 76 | 0.01 | 1.0E+00 |
| | PUL 53 | | | | | |
| Bova115_2289 | Predicted | Cell surface glycan- | SusD | 91 | 0.03 | 1.0E+00 |
| | PUL 53 | binding lipoprotein, | | | | |
| | | SusD family | | | | |
| Bova115_2290 | Predicted | Outer membrane | Trans | 96 | 0.03 | 9.0E−01 |
| | PUL 53 | TonB-dependent | | | | |
| | | transporter, SusC | | | | |
| | | family | | | | |
| Bova115_2291 | Predicted | hypothetical protein | REG | 99 | −0.08 | 6.9E−01 |
| | PUL 53 | | | | | |
| Bova115_2292 | Predicted | hypothetical protein | REG | 100 | −0.09 | 6.6E−01 |
| | PUL 53 | | | | | |
| Bova115_2318 | Predicted | Alkaline phosphatase | | 99 | −0.02 | 1.0E+00 |
| | PUL 55 | (EC 3.1.3.1) | | | | |
| Bova115_2319 | Predicted | hypothetical protein | | 98 | −0.05 | 1.0E+00 |
| | PUL 55 | | | | | |
| Bova115_2320 | Predicted | Alpha-1,2- | GH92 | 99 | −0.05 | 1.0E+00 |
| | PUL 55 | mannosidase | | | | |
| Bova115_2331 | Predicted | hypothetical protein | | 100 | 0.02 | 1.0E+00 |
| | PUL 56 | | | | | |
| Bova115_2332 | Predicted | hypothetical protein | Pept | 100 | −0.01 | 1.0E+00 |
| | PUL 56 | | | | | |
| Bova115_2333 | Predicted | hypothetical protein | SusD | 100 | 0.02 | 1.0E+00 |
| | PUL 56 | | | | | |
| Bova115_2334 | Predicted | Outer membrane | Trans | 99 | 0.02 | 9.4E−01 |
| | PUL 56 | TonB-dependent | | | | |
| | | transporter, SusC | | | | |
| | | family | | | | |
| Bova115_2335 | Predicted | hypothetical protein | | 99 | 0.09 | 1.0E+00 |
| | PUL 56 | | | | | |
| Bova115_2354 | Predicted | arylsulfatase A | Sulf | 99 | −0.05 | 1.0E+00 |
| | PUL 58 | precursor | | | | |
| Bova115_2355 | Predicted | hypothetical protein | | 98 | 0.00 | 1.0E+00 |
| | PUL 58 | | | | | |
| Bova115_2356 | Predicted | hypothetical protein | | 98 | 0.02 | 1.0E+00 |
| | PUL 58 | | | | | |
| Bova115_2357 | Predicted | hypothetical protein | | 84 | 0.06 | 1.0E+00 |
| | PUL 58 | | | | | |
| Bova115_2358 | Predicted | putative beta- | GH43_31 | 90 | 0.01 | 1.0E+00 |
| | PUL 58 | xylosidase | | | | |
| Bova115_2359 | Predicted | Outer membrane | Trans | 98 | −0.09 | 5.8E−01 |
| | PUL 58 | TonB-dependent | | | | |
| | | transporter, SusC | | | | |
| | | family | | | | |
| Bova115_2360 | Predicted | Cell surface glycan- | SusD | 99 | 0.00 | 1.0E+00 |
| | PUL 58 | binding lipoprotein, | | | | |
| | | SusD family | | | | |
| Bova115_2361 | Predicted | Outer membrane | Trans | 99 | −0.03 | 1.0E+00 |
| | PUL 58 | TonB-dependent | | | | |
| | | transporter, SusC | | | | |
| | | family | | | | |
| Bova115_2362 | Predicted | Cell surface glycan- | SusD | 99 | 0.03 | 1.0E+00 |
| | PUL 58 | binding lipoprotein, | | | | |
| | | SusD family | | | | |
| Bova115_2363 | Predicted | hypothetical protein | | 99 | 0.00 | 1.0E+00 |
| | PUL 58 | | | | | |
| Bova115_2364 | Predicted | BaeS-type histidine | REG | 99 | −0.01 | 1.0E+00 |
| | PUL 58 | kinase/OmpR-type | | | | |
| | | DNA-binding response | | | | |
| | | regulator | | | | |
| Bova115_2365 | Predicted | alpha-L- | GH51 | 100 | 0.04 | 1.0E+00 |
| | PUL 58 | arabinofuranosidase | | | | |
| | | (EC 3.2.1.55) | | | | |
| Bova115_2366 | Predicted | Putative sulfatase | Sulf | 100 | 0.03 | 1.0E+00 |
| | PUL 58 | | | | | |
| Bova115_2367 | Predicted | putative secreted | GH43 | 99 | 0.01 | 1.0E+00 |
| | PUL 58 | xylosidase | | | | |
| Bova115_2368 | Predicted | Arylsulfatase (EC | Sulf | 100 | 0.03 | 1.0E+00 |
| | PUL 58 | 3.1.6.1) | | | | |
| Bova115_2369 | Predicted | beta-galactosidase | GH2 | 99 | −0.04 | 1.0E+00 |
| | PUL 58 | (EC 3.2.1.23) | | | | |
| Bova115_2372 | Predicted | Regulatory protein | REG | 100 | −0.09 | 1.0E+00 |
| | PUL 59 | SusR | | | | |
| Bova115_2373 | Predicted | Outer membrane | Trans | 100 | 0.02 | 1.0E+00 |
| | PUL 59 | TonB-dependent | | | | |
| | | transporter, SusC | | | | |
| | | family | | | | |
| Bova115_2374 | Predicted | Cell surface glycan- | SusD | 100 | 0.00 | 1.0E+00 |
| | PUL 59 | binding lipoprotein, | | | | |
| | | SusD family | | | | |
| Bova115_2375 | Predicted | hypothetical protein | | 98 | −0.04 | 1.0E+00 |
| | PUL 59 | | | | | |
| Bova115_2376 | Predicted | dextranase precursor | GH66 | 99 | 0.04 | 1.0E+00 |
| | PUL 59 | | | | | |
| Bova115_2377 | Predicted | alpha-xylosidase (EC | GH31 | 97 | −0.02 | 1.0E+00 |
| | PUL 59 | 3.2.1.177) | | | | |
| Bova115_2027 | Predicted | Cell surface glycan- | SusD | 60 | 0.03 | 1.0E+00 |
| | PUL 45 | binding lipoprotein, | | | | |
| | | SusD family | | | | |
| Bova115_2028 | Predicted | Outer membrane | Trans | 60 | 0.00 | 1.0E+00 |
| | PUL 45 | TonB-dependent | | | | |
| | | transporter, SusC | | | | |
| | | family | | | | |
| Bova115_2029 | Predicted | Cell surface glycan- | SusD | 57 | 0.02 | 1.0E+00 |
| | PUL 45 | binding lipoprotein, | | | | |
| | | SusD family | | | | |
| Bova115_2030 | Predicted | Outer membrane | Trans | 65 | −0.05 | 1.0E+00 |
| | PUL 45 | TonB-dependent | | | | |
| | | transporter, SusC | | | | |
| | | family | | | | |
| Bova115_2341 | Predicted | alpha-L-fucosidase | GH29 | 42 | 0.00 | 1.0E+00 |
| | PUL 57 | (EC 3.2.1.51) | | | | |
| Bova115_2379 | Predicted | Transcriptional | REG | 94 | −0.04 | 1.0E+00 |
| | PUL 60 | regulator, GntR family | | | | |
| Bova115_2381 | Predicted | Outer membrane | Trans | 88 | −0.01 | 1.0E+00 |
| | PUL 60 | TonB-dependent | | | | |
| | | transporter, SusC | | | | |
| | | family | | | | |
| Bova115_2382 | Predicted | Cell surface glycan- | SusD | 84 | 0.00 | 1.0E+00 |
| | PUL 60 | binding lipoprotein, | | | | |
| | | SusD family | | | | |
| Bova115_2383 | Predicted | hypothetical protein | | 62 | −0.02 | 1.0E+00 |
| | PUL 60 | | | | | |
| Bova115_2384 | Predicted | Alginate lyase | PL6_1 | 92 | −0.04 | 1.0E+00 |
| | PUL 60 | precursor (EC 4.2.2.3) | | | | |
| Bova115_2385 | Predicted | hypothetical protein | PL17 2 | 88 | 0.04 | 1.0E+00 |
| | PUL 60 | | | | | |
| Bova115_2386 | | Pectin degradation | | 97 | 0.02 | 1.0E+00 |
| | | protein KdgF | | | | |
| Bova115_2387 | | Predicted | Trans | 96 | 0.01 | 1.0E+00 |
| | | mannuronate | | | | |
| | | transporter | | | | |
| Bova115_2388 | | Acetoin(diacetyl) | | 99 | −0.02 | 1.0E+00 |
| | | reductase | | | | |
| Bova115_2389 | | hypothetical protein | PL38 | 94 | 0.01 | 1.0E+00 |
| Bova115_2390 | | putative esterase | | 93 | 0.01 | 1.0E+00 |
| Bova115_2391 | | hypothetical protein | | 86 | −0.01 | 1.0E+00 |
| Bova115_2395 | | Putative lipase | | 96 | −0.01 | 1.0E+00 |
| Bova115_2396 | | Cysteine synthase | | 99 | −0.11 | 5.6E−01 |
| | | (EC 2.5.1.47) | | | | |
| Bova115_2397 | Predicted | beta-glucosidase (EC | GH3 | 99 | 0.03 | 1.0E+00 |
| | PUL 61 | 3.2.1.21) | | | | |
| Bova115_2398 | Predicted | hypothetical protein | | 94 | 0.01 | 1.0E+00 |
| | PUL 61 | | | | | |
| Bova115_2399 | Predicted | Cell surface glycan- | SusD | 98 | 0.00 | 1.0E+00 |
| | PUL 61 | binding lipoprotein, | | | | |
| | | SusD family | | | | |
| Bova115_2400 | Predicted | Outer membrane | Trans | 100 | −0.03 | 1.0E+00 |
| | PUL 61 | TonB-dependent | | | | |
| | | transporter, SusC | | | | |
| | | family | | | | |
| Bova115_2401 | Predicted | Beta-1,3(4)-glucanase | GH16 | 99 | −0.04 | 1.0E+00 |
| | PUL 61 | precursor (EC 3.2.1.6) | | | | |
| Bova115_2402 | Predicted | hypothetical protein | REG | 99 | 0.03 | 9.2E−01 |
| | PUL 61 | | | | | |
| Bova115_2403 | Predicted | hypothetical protein | | 95 | — | — |
| | PUL 61 | | | | | |
| Bova115_2405 | Predicted | beta-glucosidase (EC | GH3 | 98 | −0.01 | 1.0E+00 |
| | PUL 61 | 3.2.1.21) | | | | |
| Bova115_2410 | | Pyridoxamine 5′- | | 94 | −0.03 | 1.0E+00 |
| | | phosphate oxidase- | | | | |
| | | related, FMN-binding | | | | |
| Bova115_2411 | | 3′-to-5′ | | 100 | −0.18 | 1.9E−01 |
| | | exoribonuclease | | | | |
| | | RNase R | | | | |
| Bova115_2412 | Predicted | hypothetical protein | | 99 | −0.12 | 1.0E+00 |
| | PUL 62 | | | | | |
| Bova115_2413 | Predicted | hypothetical protein | | 98 | 0.03 | 1.0E+00 |
| | PUL 62 | | | | | |
| Bova115_2414 | Predicted | FIG00938555: | | 98 | 0.04 | 1.0E+00 |
| | PUL 62 | hypothetical protein | | | | |
| Bova115_2415 | Predicted | FIG00939679: | | 99 | 0.02 | 1.0E+00 |
| | PUL 62 | hypothetical protein | | | | |
| Bova115_2416 | Predicted | hypothetical protein | Pept | 99 | −0.01 | 1.0E+00 |
| | PUL 62 | | | | | |
| Bova115_2417 | Predicted | Cell surface glycan- | SusD | 98 | 0.00 | 1.0E+00 |
| | PUL 62 | binding lipoprotein, | | | | |
| | | SusD family | | | | |
| Bova115_2418 | Predicted | Outer membrane | Trans | 100 | −0.09 | 6.2E−01 |
| | PUL 62 | TonB-dependent | | | | |
| | | transporter, SusC | | | | |
| | | family | | | | |
| Bova115_2419 | Predicted | Putative anti-sigma | REG | 98 | −0.02 | 1.0E+00 |
| | PUL 62 | factor | | | | |
| Bova115_2420 | Predicted | hypothetical protein | REG | 99 | 0.08 | 6.4E−01 |
| | PUL 62 | | | | | |
| Bova115_2449 | Predicted | Chitinase (EC | GH18 | 91 | 0.01 | 1.0E+00 |
| | PUL 63 | 3.2.1.14) | | | | |
| Bova115_2450 | Predicted | BaeS-type histidine | REG | 93 | −0.14 | 4.7E−01 |
| | PUL 63 | kinase/OmpR-type | | | | |
| | | DNA-binding response | | | | |
| | | regulator | | | | |
| Bova115_2451 | Predicted | hypothetical protein | | 86 | — | — |
| | PUL 63 | | | | | |
| Bova115_2452 | Predicted | Outer membrane | Trans | 98 | 0.01 | 1.0E+00 |
| | PUL 63 | TonB-dependent | | | | |
| | | transporter, SusC | | | | |
| | | family | | | | |
| Bova115_2453 | Predicted | Cell surface glycan- | SusD | 95 | −0.05 | 1.0E+00 |
| | PUL 63 | binding lipoprotein, | | | | |
| | | SusD family | | | | |
| Bova115_2454 | Predicted | hypothetical protein | | 95 | −0.05 | 1.0E+00 |
| | PUL 63 | | | | | |
| Bova115_2455 | Predicted | Hypothetical glycoside | GH43_34 | 94 | 0.03 | 1.0E+00 |
| | PUL 63 | hydrolase, family 43, | | | | |
| | | similar to | | | | |
| | | arabinosidase | | | | |
| Bova115_2456 | Predicted | putative xylanase | GH30_4 | 90 | −0.06 | 1.0E+00 |
| | PUL 63 | | | | | |
| Bova115_406 | Predicted | alpha-xylosidase (EC | GH31 | 72 | 0.02 | 1.0E+00 |
| | PUL 11 | 3.2.1.177) | | | | |
| Bova115_2464 | Predicted | Sialic acid-specific 9- | | 99 | 0.00 | 1.0E+00 |
| | PUL 64 | O-acetylesterase | | | | |
| Bova115_2465 | Predicted | hypothetical protein | | 98 | −0.05 | 1.0E+00 |
| | PUL 64 | | | | | |
| Bova115_2466 | Predicted | alpha-rhamnosidase | GH78 | 99 | −0.07 | 1.0E+00 |
| | PUL 64 | | | | | |
| Bova115_2467 | Predicted | Sodium: galactoside | Trans | 99 | 0.01 | 1.0E+00 |
| | PUL 64 | symporter family | | | | |
| | | protein | | | | |
| Bova115_2468 | Predicted | 4-O-beta-D-mannosyl- | GH130 | 98 | 0.05 | 1.0E+00 |
| | PUL 64 | D-glucose | | | | |
| | | phosphorylase (EC | | | | |
| | | 2.4.1.281) | | | | |
| Bova115_2469 | Predicted | hypothetical protein | GH140 | 94 | −0.01 | 1.0E+00 |
| | PUL 64 | | | | | |
| Bova115_2470 | Predicted | hypothetical protein | GH5_5 | 97 | 0.01 | 1.0E+00 |
| | PUL 64 | | | | | |
| Bova115_2471 | Predicted | beta-galactosidase | GH2 | 96 | −0.06 | 8.0E−01 |
| | PUL 64 | (EC 3.2.1.23) | | | | |
| Bova115_2472 | Predicted | hypothetical protein | GH5_5 | 99 | −0.03 | 9.0E−01 |
| | PUL 64 | | | | | |
| Bova115_2473 | Predicted | hypothetical protein | | 98 | −0.04 | 8.4E−01 |
| | PUL 64 | | | | | |
| Bova115_2474 | Predicted | Cell surface glycan- | SusD | 98 | 0.04 | 1.0E+00 |
| | PUL 64 | binding lipoprotein, | | | | |
| | | SusD family | | | | |
| Bova115_2475 | Predicted | Outer membrane | Trans | 99 | 0.03 | 1.0E+00 |
| | PUL 64 | TonB-dependent | | | | |
| | | transporter, SusC | | | | |
| | | family | | | | |
| Bova115_2476 | Predicted | hypothetical protein | GH5_2 | 98 | −0.01 | 1.0E+00 |
| | PUL 64 | | | | | |
| Bova115_2516 | Predicted | Outer membrane | Trans | 98 | 0.06 | 8.2E−01 |
| | PUL 65 | TonB-dependent | | | | |
| | | transporter, SusC | | | | |
| | | family | | | | |
| Bova115_2517 | Predicted | Cell surface glycan- | SusD | 95 | −0.01 | 1.0E+00 |
| | PUL 65 | binding lipoprotein, | | | | |
| | | SusD family | | | | |
| Bova115_2518 | Predicted | hypothetical protein | GH115 | 99 | −0.03 | 9.1E−01 |
| | PUL 65 | | | | | |
| Bova115_2519 | Predicted | hypothetical protein | | 96 | 0.02 | 9.2E−01 |
| | PUL 65 | | | | | |
| Bova115_2520 | Predicted | hypothetical protein | GH105 | 98 | 0.05 | 1.0E+00 |
| | PUL 65 | | | | | |
| Bova115_2524 | Predicted | hypothetical protein | GH43_3 | 63 | 0.00 | 1.0E+00 |
| | PUL 65 | | | | | |
| Bova115_2525 | Predicted | hypothetical protein | | 41 | −0.01 | 1.0E+00 |
| | PUL 65 | | | | | |
| Bova115_2526 | Predicted | Outer membrane | Trans | 68 | 0.10 | 6.5E−01 |
| | PUL 65 | TonB-dependent | | | | |
| | | transporter, SusC | | | | |
| | | family | | | | |
| Bova115_2527 | Predicted | Cell surface glycan- | SusD | 61 | −0.04 | 1.0E+00 |
| | PUL 65 | binding lipoprotein, | | | | |
| | | SusD family | | | | |
| Bova115_2528 | Predicted | hypothetical protein | | 51 | −0.01 | 1.0E+00 |
| | PUL 65 | | | | | |
| Bova115_2533 | Predicted | hypothetical protein | | 89 | −0.01 | 1.0E+00 |
| | PUL 65 | | | | | |
| Bova115_2534 | Predicted | hypothetical protein | | 94 | 0.03 | 1.0E+00 |
| | PUL 65 | | | | | |
| Bova115_2535 | Predicted | Beta-mannosidase | GH2 | 99 | −0.04 | 1.0E+00 |
| | PUL 65 | (EC 3.2.1.25) | | | | |
| Bova115_2536 | Predicted | FIG01423360: | GH130 | 95 | −0.05 | 1.0E+00 |
| | PUL 65 | glycoside hydrolase | | | | |
| Bova115_2537 | Predicted | Beta-mannosidase | GH2 | 93 | 0.03 | 1.0E+00 |
| | PUL 65 | (EC 3.2.1.25) | | | | |
| Bova115_2538 | Predicted | hypothetical protein | GH105 | 88 | 0.03 | 1.0E+00 |
| | PUL 65 | | | | | |
| Bova115_2551 | Predicted | RNA polymerase | REG | 80 | 0.03 | 1.0E+00 |
| | PUL 66 | ECF-type sigma factor | | | | |
| Bova115_2552 | Predicted | Putative anti-sigma | REG | 74 | 0.04 | 9.0E−01 |
| | PUL 66 | factor | | | | |
| Bova115_2553 | Predicted | Outer membrane | Trans | 74 | 0.01 | 1.0E+00 |
| | PUL 66 | TonB-dependent | | | | |
| | | transporter, SusC | | | | |
| | | family | | | | |
| Bova115_2554 | Predicted | Cell surface glycan- | SusD | 57 | −0.06 | 1.0E+00 |
| | PUL 66 | binding lipoprotein, | | | | |
| | | SusD family | | | | |
| Bova115_2570 | Predicted | hypothetical protein | | 35 | −0.02 | 1.0E+00 |
| | PUL 67 | | | | | |
| Bova115_2557 | Predicted | hypothetical protein | | 59 | 0.02 | 1.0E+00 |
| | PUL 66 | | | | | |
| Bova115_2571 | Predicted | hypothetical protein | | 51 | 0.05 | 1.0E+00 |
| | PUL 67 | | | | | |
| Bova115_2578 | Predicted | beta-glucosidase (EC | GH3 | 88 | −0.01 | 1.0E+00 |
| | PUL 67 | 3.2.1.21) | | | | |
| Bova115_2579 | Predicted | beta-glucosidase (EC | GH3 | 92 | −0.04 | 1.0E+00 |
| | PUL 67 | 3.2.1.21) | | | | |
| Bova115_2564 | Predicted | Rhamnogalacturonides | GH105 | 94 | 0.05 | 1.0E+00 |
| | PUL 67 | degradation protein | | | | |
| | | RhiN | | | | |
| Bova115_2572 | Predicted | hypothetical protein | | 93 | 0.00 | 1.0E+00 |
| | PUL 67 | | | | | |
| Bova115_2573 | Predicted | alpha-galactosidase | | 91 | 0.04 | 1.0E+00 |
| | PUL 67 | (EC 3.2.1.22) | | | | |
| Bova115_2574 | Predicted | hypothetical protein | CE7 | 91 | 0.05 | 1.0E+00 |
| | PUL 67 | | | | | |
| Bova115_2575 | Predicted | beta-galactosidase | GH2 | 96 | 0.00 | 1.0E+00 |
| | PUL 67 | (EC 3.2.1.23) | | | | |
| Bova115_2577 | Predicted | hypothetical protein | | 91 | 0.00 | 1.0E+00 |
| | PUL 67 | | | | | |
| Bova115_2765 | Predicted | Two-component | REG | 99 | 0.32 | 1.6E−01 |
| | PUL 70 | system sensor | | | | |
| | | histidine | | | | |
| | | kinase/response | | | | |
| | | regulator hybrid | | | | |
| Bova115_2766 | Predicted | Outer membrane | Trans | 100 | −0.06 | 8.3E−01 |
| | PUL 70 | TonB-dependent | | | | |
| | | transporter, SusC | | | | |
| | | family | | | | |
| Bova115_2767 | Predicted | Cell surface glycan- | SusD | 99 | 0.00 | 1.0E+00 |
| | PUL 70 | binding lipoprotein, | | | | |
| | | SusD family | | | | |
| Bova115_2768 | Predicted | hypothetical protein | | 85 | 0.01 | 1.0E+00 |
| | PUL 70 | | | | | |
| Bova115_2769 | Predicted | spore peptidoglycan | GH | 79 | 0.00 | 1.0E+00 |
| | PUL 70 | hydrolase (N- | | | | |
| | | acetylglucosaminidase) | | | | |
| Bova115_2772 | Predicted | Sugar: proton | | 67 | −0.04 | 1.0E+00 |
| | PUL 71 | symporter | | | | |
| Bova115_1757 | Predicted | hypothetical protein | CE | 30 | 0.02 | 1.0E+00 |
| | PUL 38 | | | | | |
| Bova115_2779 | Predicted | Ribokinase (EC | | 55 | −0.01 | 1.0E+00 |
| | PUL 71 | 2.7.1.15) | | | | |
| Bova115_2784 | Predicted | Outer membrane | Trans | 99 | 0.03 | 9.3E−01 |
| | PUL 72 | TonB-dependent | | | | |
| | | transporter, SusC | | | | |
| | | family | | | | |
| Bova115_2785 | Predicted | Cell surface glycan- | SusD | 100 | 0.04 | 1.0E+00 |
| | PUL 72 | binding lipoprotein, | | | | |
| | | SusD family | | | | |
| Bova115_2786 | Predicted | probable secreted | | 100 | 0.05 | 1.0E+00 |
| | PUL 72 | protein | | | | |
| Bova115_2787 | Predicted | Alpha-N- | GH89 | 99 | −0.02 | 1.0E+00 |
| | PUL 72 | acetylglucosaminidase | | | | |
| | | (EC 3.2.1.50) | | | | |
| Bova115_2788 | Predicted | Glycerate kinase (EC | | 97 | −0.01 | 1.0E+00 |
| | PUL 72 | 2.7.1.31) | | | | |
| Bova115_2789 | Predicted | D-glycerate | | 99 | −0.01 | 1.0E+00 |
| | PUL 72 | transporter (predicted) | | | | |
| Bova115_2790 | Predicted | N-acetylglucosamine | | 99 | 0.02 | 1.0E+00 |
| | PUL 72 | related transporter, | | | | |
| | | NagX | | | | |
| Bova115_2791 | Predicted | Alpha-N- | GH89 | 98 | 0.04 | 1.0E+00 |
| | PUL 72 | acetylglucosaminidase | | | | |
| | | (EC 3.2.1.50) | | | | |
| Bova115_2792 | Predicted | RNA polymerase | REG | 100 | 0.09 | 1.0E+00 |
| | PUL 72 | ECF-type sigma factor | | | | |
| Bova115_2793 | Predicted | Putative anti-sigma | REG | 99 | 0.04 | 8.8E−01 |
| | PUL 72 | factor | | | | |
| Bova115_2797 | Predicted | Putative anti-sigma | REG | 99 | 0.05 | 1.0E+00 |
| | PUL 73 | factor | | | | |
| Bova115_2798 | Predicted | Outer membrane | Trans | 100 | −0.02 | 1.0E+00 |
| | PUL 73 | TonB-dependent | | | | |
| | | transporter, SusC | | | | |
| | | family | | | | |
| Bova115_2799 | Predicted | hypothetical protein | SusD | 100 | 0.03 | 1.0E+00 |
| | PUL 73 | | | | | |
| Bova115_2800 | Predicted | putative cellulase | GH5_13 | 99 | −0.02 | 1.0E+00 |
| | PUL 73 | family | | | | |
| Bova115_2801 | Predicted | hypothetical protein | GH | 99 | 0.01 | 1.0E+00 |
| | PUL 73 | | | | | |
| Bova115_2802 | Predicted | hypothetical protein | | 98 | 0.08 | 1.0E+00 |
| | PUL 73 | | | | | |
| Bova115_2803 | Predicted | Uncharacterized | | 98 | −0.09 | 6.7E−01 |
| | PUL 73 | protein YphG, TPR- | | | | |
| | | domain containing | | | | |
| Bova115_2804 | Predicted | RNA polymerase | REG | 99 | 0.04 | 8.8E−01 |
| | PUL 73 | ECF-type sigma factor | | | | |
| Bova115_2805 | Predicted | L-arabinose-specific | EPI | 97 | 0.01 | 1.0E+00 |
| | PUL 73 | 1-epimerase | | | | |
| | | (mutarotase) | | | | |
| Bova115_5607 | Predicted | Cell surface glycan- | SusD | 32 | 0.03 | 1.0E+00 |
| | PUL 109 | binding lipoprotein, | | | | |
| | | SusD family | | | | |
| Bova115_109 | Predicted | Alpha-1,2- | GH92-GH88 | 63 | 0.03 | 9.0E−01 |
| | PUL 2 | mannosidase | | | | |
| Bova115_3118 | Predicted | Putative anti-sigma | REG | 92 | −0.04 | 8.5E−01 |
| | PUL 75 | factor | | | | |
| Bova115_3119 | Predicted | RNA polymerase | REG | 97 | 0.05 | 1.0E+00 |
| | PUL 75 | ECF-type sigma factor | | | | |
| Bova115_3124 | Predicted | putative cellulase | GH5_13 | 96 | −0.02 | 1.0E+00 |
| | PUL 75 | family | | | | |
| Bova115_3125 | Predicted | beta-N- | GH20 | 91 | 0.02 | 1.0E+00 |
| | PUL 75 | acetylglucosaminidase | | | | |
| | | (EC 3.2.1.52) | | | | |
| Bova115_3129 | Predicted | RNA polymerase | REG | 99 | 0.00 | 1.0E+00 |
| | PUL 76 | ECF-type sigma factor | | | | |
| Bova115_3130 | Predicted | Putative anti-sigma | REG | 100 | 0.11 | 1.0E+00 |
| | PUL 76 | factor | | | | |
| Bova115_3131 | Predicted | FIG00410220: | | 100 | 0.01 | 1.0E+00 |
| | PUL 76 | hypothetical protein | | | | |
| Bova115_3132 | Predicted | Outer membrane | Trans | 99 | 0.00 | 1.0E+00 |
| | PUL 76 | TonB-dependent | | | | |
| | | transporter, SusC | | | | |
| | | family | | | | |
| Bova115_3133 | Predicted | Cell surface glycan- | SusD | 96 | 0.02 | 1.0E+00 |
| | PUL 76 | binding lipoprotein, | | | | |
| | | SusD family | | | | |
| Bova115_3134 | Predicted | hypothetical protein | GH43_18 | 100 | 0.00 | 1.0E+00 |
| | PUL 76 | | | | | |
| Bova115_3135 | Predicted | hypothetical protein | | 100 | 0.04 | 1.0E+00 |
| | PUL 76 | | | | | |
| Bova115_3136 | Predicted | beta-galactosidase | GH42 | 99 | −0.01 | 1.0E+00 |
| | PUL 76 | (EC 3.2.1.23) | | | | |
| Bova115_3137 | Predicted | putative cellulase | GH5_13 | 91 | −0.03 | 1.0E+00 |
| | PUL 76 | family | | | | |
| Bova115_3138 | Predicted | Putative alpha- | GH97 | 100 | −0.08 | 6.5E−01 |
| | PUL 76 | glucosidase | | | | |
| Bova115_3152 | | Gliding motility- | Trans | 99 | −0.01 | 1.0E+00 |
| | | associated ABC | | | | |
| | | transporter permease | | | | |
| | | protein GldF/Gliding | | | | |
| | | motility-associated | | | | |
| | | ABC transporter | | | | |
| | | substrate-binding | | | | |
| | | protein GldG | | | | |
| Bova115_3153 | | Gliding motility- | Trans | 98 | −0.04 | 8.8E−01 |
| | | associated ABC | | | | |
| | | transporter permease | | | | |
| | | protein GldF/Gliding | | | | |
| | | motility-associated | | | | |
| | | ABC transporter | | | | |
| | | substrate-binding | | | | |
| | | protein GldG | | | | |
| Bova115_3154 | | Gliding motility- | Trans | 98 | −0.03 | 1.0E+00 |
| | | associated ABC | | | | |
| | | transporter ATP- | | | | |
| | | binding protein GldA | | | | |
| Bova115_3157 | Predicted | hypothetical protein | Trans | 98 | −0.07 | 8.1E−01 |
| | PUL 77 | | | | | |
| Bova115_3158 | Predicted | Outer membrane | Trans | 53 | 0.00 | 1.0E+00 |
| | PUL 77 | TonB-dependent | | | | |
| | | transporter, SusC | | | | |
| | | family | | | | |
| Bova115_3159 | Predicted | hypothetical protein | | 98 | −0.16 | 4.4E−01 |
| | PUL 77 | | | | | |
| Bova115_3160 | Predicted | hypothetical protein | | 98 | 0.10 | 1.0E+00 |
| | PUL 77 | | | | | |
| Bova115_3161 | Predicted | hypothetical protein | | 47 | −0.04 | 8.8E−01 |
| | PUL 77 | | | | | |
| Bova115_3162 | Predicted | hypothetical protein | | 44 | 0.03 | 1.0E+00 |
| | PUL 77 | | | | | |
| Bova115_3163 | Predicted | hypothetical protein | SusD | 56 | −0.01 | 1.0E+00 |
| | PUL 77 | | | | | |
| Bova115_3164 | Predicted | Outer membrane | Trans | 82 | −0.07 | 6.9E−01 |
| | PUL 77 | TonB-dependent | | | | |
| | | transporter, SusC | | | | |
| | | family | | | | |
| Bova115_3165 | Predicted | hypothetical protein | REG | 86 | 0.06 | 1.0E+00 |
| | PUL 77 | | | | | |
| Bova115_3166 | Predicted | RNA polymerase | REG | 97 | 0.04 | 1.0E+00 |
| | PUL 77 | ECF-type sigma factor | | | | |
| Bova115_353 | Predicted | hypothetical protein | | 28 | 0.03 | 1.0E+00 |
| | PUL 10 | | | | | |
| Bova115_3229 | Predicted | Cell surface glycan- | SusD | 98 | −0.01 | 1.0E+00 |
| | PUL 78 | binding lipoprotein, | | | | |
| | | SusD family | | | | |
| Bova115_3230 | Predicted | Outer membrane | Trans | 99 | 0.00 | 1.0E+00 |
| | PUL 78 | TonB-dependent | | | | |
| | | transporter, SusC | | | | |
| | | family | | | | |
| Bova115_3261 | Predicted | Cell surface glycan- | SusD | 98 | −0.14 | 4.3E−01 |
| | PUL 80 | binding lipoprotein, | | | | |
| | | SusD family | | | | |
| Bova115_3262 | Predicted | Outer membrane | Trans | 100 | −0.02 | 9.5E−01 |
| | PUL 80 | TonB-dependent | | | | |
| | | transporter, SusC | | | | |
| | | family | | | | |
| Bova115_3265 | | Integrase | Int | 100 | −0.25 | 8.2E−02 |
| Bova115_3264 | | hypothetical protein | | 97 | — | — |
| Bova115_3266 | Predicted | Outer membrane | Trans | 100 | 0.02 | 9.3E−01 |
| | PUL 81 | TonB-dependent | | | | |
| | | transporter, SusC | | | | |
| | | family | | | | |
| Bova115 3267 | Predicted | Cell surface glycan- | SusD | 100 | 0.11 | 5.8E−01 |
| | PUL 81 | binding lipoprotein, | | | | |
| | | SusD family | | | | |
| Bova115_3301 | Predicted | Uncharacterized | | 99 | 0.05 | 1.0E+00 |
| | PUL 83 | protein YphG, TPR- | | | | |
| | | domain containing | | | | |
| Bova115_3302 | Predicted | hypothetical protein | CBM61- | 96 | 0.03 | 1.0E+00 |
| | PUL 83 | | GH53 | | | |
| Bova115_3303 | Predicted | putative lipoprotein | | 96 | −0.01 | 1.0E+00 |
| | PUL 83 | | | | | |
| Bova115_3304 | Predicted | Outer membrane | Trans | 98 | 0.02 | 1.0E+00 |
| | PUL 83 | TonB-dependent | | | | |
| | | transporter, SusC | | | | |
| | | family | | | | |
| Bova115_3305 | Predicted | Cell surface glycan- | SusD | 99 | −0.01 | 1.0E+00 |
| | PUL 83 | binding lipoprotein, | | | | |
| | | SusD family | | | | |
| Bova115_3306 | Predicted | hypothetical protein | | 98 | 0.00 | 1.0E+00 |
| | PUL 83 | | | | | |
| Bova115 3307 | Predicted | beta-galactosidase | GH35 | 98 | −0.03 | 1.0E+00 |
| | PUL 83 | (EC 3.2.1.23) | | | | |
| Bova115_3309 | Predicted | hypothetical protein | GH147 | 97 | 0.02 | 1.0E+00 |
| | PUL 83 | | | | | |
| Bova115_3420 | Predicted | Predicted | REG | 99 | −0.13 | 4.5E−01 |
| | PUL 85 | transcriptional | | | | |
| | | regulator of N- | | | | |
| | | Acetylglucosamine | | | | |
| | | utilization, GntR family | | | | |
| Bova115_3421 | Predicted | mannose-6-phosphate | | 98 | 0.21 | 2.5E−01 |
| | PUL 85 | isomerase, class I | | | | |
| Bova115_3422 | Predicted | ROK family sugar | ROK | 64 | −0.02 | 1.0E+00 |
| | PUL 85 | kinase or | | | | |
| | | transcriptional | | | | |
| | | regulator | | | | |
| Bova115_3428 | Predicted | Alpha-1,2- | GH92 | 32 | 0.08 | 1.0E+00 |
| | PUL 85 | mannosidase | | | | |
| Bova115_3427 | Predicted | Glucose/galactose | Trans | 73 | 0.00 | 1.0E+00 |
| | PUL 85 | transporter | | | | |
| Bova115_3512 | Predicted | Cell surface glycan- | SusD | 98 | 0.01 | 1.0E+00 |
| | PUL 87 | binding lipoprotein, | | | | |
| | | SusD family | | | | |
| Bova115_3513 | Predicted | Outer membrane | Trans | 99 | −0.01 | 1.0E+00 |
| | PUL 87 | TonB-dependent | | | | |
| | | transporter, SusC | | | | |
| | | family | | | | |
| Bova115_3546 | Predicted | hypothetical protein | REG | 99 | 0.02 | 1.0E+00 |
| | PUL 88 | | | | | |
| Bova115_3547 | Predicted | Putative anti-sigma | REG | 99 | −0.05 | 1.0E+00 |
| | PUL 88 | factor | | | | |
| Bova115_3548 | Predicted | Outer membrane | Trans | 100 | −0.02 | 1.0E+00 |
| | PUL 88 | TonB-dependent | | | | |
| | | transporter, SusC | | | | |
| | | family | | | | |
| Bova115_3549 | Predicted | Cell surface glycan- | SusD | 99 | −0.04 | 1.0E+00 |
| | PUL 88 | binding lipoprotein, | | | | |
| | | SusD family | | | | |
| Bova115_3550 | Predicted | hypothetical protein | | 99 | −0.01 | 1.0E+00 |
| | PUL 88 | | | | | |
| Bova115_3551 | Predicted | beta-N- | GH20 | 100 | 0.03 | 1.0E+00 |
| | PUL 88 | acetylglucosaminidase | | | | |
| | | (EC 3.2.1.52) | | | | |
| Bova115_3552 | Predicted | beta-glycosyl | GH20 | 100 | 0.00 | 1.0E+00 |
| | PUL 88 | hydrolase | | | | |
| Bova115_3553 | Predicted | N-acetylglucosamine | | 98 | 0.00 | 1.0E+00 |
| | PUL 88 | related transporter, | | | | |
| | | NagX | | | | |
| Bova115_3560 | Predicted | hypothetical protein | | 99 | −0.08 | 1.0E+00 |
| | PUL 89 | | | | | |
| Bova115_3561 | Predicted | hypothetical protein | | 98 | 0.04 | 8.8E−01 |
| | PUL 89 | | | | | |
| Bova115_3562 | Predicted | Cell surface glycan- | SusD | 100 | 0.01 | 1.0E+00 |
| | PUL 89 | binding lipoprotein, | | | | |
| | | SusD family | | | | |
| Bova115 3563 | Predicted | Outer membrane | Trans | 100 | 0.00 | 1.0E+00 |
| | PUL 89 | TonB-dependent | | | | |
| | | transporter, SusC | | | | |
| | | family | | | | |
| Bova115_3579 | Predicted | Outer membrane | Trans | 100 | 0.03 | 1.0E+00 |
| | PUL 90 | TonB-dependent | | | | |
| | | transporter, SusC | | | | |
| | | family | | | | |
| Bova115_3580 | Predicted | Cell surface glycan- | SusD | 100 | 0.01 | 1.0E+00 |
| | PUL 90 | binding lipoprotein, | | | | |
| | | SusD family | | | | |
| Bova115_3581 | Predicted | hypothetical protein | Pept | 100 | — | — |
| | PUL 90 | | | | | |
| Bova115_3582 | Predicted | hypothetical protein | | 97 | — | — |
| | PUL 90 | | | | | |
| Bova115_3583 | Predicted | hypothetical protein | | 98 | 0.00 | 1.0E+00 |
| | PUL 90 | | | | | |
| Bova115_2324 | Predicted | hypothetical protein | | 58 | 0.00 | 1.0E+00 |
| | PUL 55 | | | | | |
| Bova115_1698 | | hypothetical protein | | 26 | −0.46 | 2.3E−02 |
| Bova115_2323 | Predicted | hypothetical protein | | 26 | −0.02 | 1.0E+00 |
| | PUL 55 | | | | | |
| Bova115_2322 | Predicted | Cell surface glycan- | SusD | 27 | −0.05 | 1.0E+00 |
| | PUL 55 | binding lipoprotein, | | | | |
| | | SusD family | | | | |
| Bova115_2321 | Predicted | Outer membrane | Trans | 35 | 0.09 | 5.8E−01 |
| | PUL 55 | TonB-dependent | | | | |
| | | transporter, SusC | | | | |
| | | family | | | | |
| Bova115_928 | | hypothetical protein | | 27 | 0.03 | 9.0E−01 |
| Bova115_3904 | Predicted | Levanase (EC | GH32 | 92 | −0.16 | 4.2E−01 |
| | PUL 91 | 3.2.1.65) | | | | |
| Bova115_3905 | Predicted | LacX protein, plasmid | EPI | 90 | −0.01 | 1.0E+00 |
| | PUL 91 | | | | | |
| Bova115_3906 | Predicted | Outer membrane | Trans | 43 | −0.20 | 5.7E−02 |
| | PUL 91 | TonB-dependent | | | | |
| | | transporter, SusC | | | | |
| | | family | | | | |
| Bova115_3907 | Predicted | Cell surface glycan- | SusD | 40 | 0.00 | 1.0E+00 |
| | PUL 91 | binding lipoprotein, | | | | |
| | | SusD family | | | | |
| Bova115_3909 | Predicted | hypothetical protein | GH91 | 25 | 0.01 | 1.0E+00 |
| | PUL 91 | | | | | |
| Bova115_3910 | Predicted | Levanase (EC | GH32 | 85 | −0.04 | 8.8E−01 |
| | PUL 91 | 3.2.1.65) | | | | |
| Bova115_3975 | Predicted | Outer membrane | Trans | 65 | −0.41 | 8.9E−02 |
| | PUL 92 | TonB-dependent | | | | |
| | | transporter, SusC | | | | |
| | | family | | | | |
| Bova115_3976 | Predicted | Cell surface glycan- | SusD | 50 | −0.08 | 6.5E−01 |
| | PUL 92 | binding lipoprotein, | | | | |
| | | SusD family | | | | |
| Bova115_3999 | Predicted | Pullulanase (EC | GH13_14 | 99 | −0.05 | 8.3E−01 |
| | PUL 93 | 3.2.1.41) | | | | |
| Bova115_4000 | Predicted | BaeS-type histidine | REG | 100 | −0.44 | 8.8E−02 |
| | PUL 93 | kinase/OmpR-type | | | | |
| | | DNA-binding response | | | | |
| | | regulator | | | | |
| Bova115_4001 | Predicted | Outer membrane | Trans | 100 | 0.03 | 1.0E+00 |
| | PUL 93 | TonB-dependent | | | | |
| | | transporter, SusC | | | | |
| | | family | | | | |
| Bova115_4002 | Predicted | Cell surface glycan- | SusD | 100 | 0.03 | 1.0E+00 |
| | PUL 93 | binding lipoprotein, | | | | |
| | | SusD family | | | | |
| Bova115_4003 | Predicted | hypothetical protein | | 100 | 0.01 | 1.0E+00 |
| | PUL 93 | | | | | |
| Bova115_4004 | Predicted | Endo-1,4-beta- | CBM4- | 98 | −0.02 | 1.0E+00 |
| | PUL 93 | xylanase (EC 3.2.1.8) | GH10 | | | |
| Bova115_4005 | Predicted | Sialic acid-specific 9- | | 100 | −0.04 | 1.0E+00 |
| | PUL 93 | O-acetylesterase | | | | |
| Bova115_4006 | Predicted | Xyloside transporter | Trans | 100 | 0.01 | 1.0E+00 |
| | PUL 93 | XynT | | | | |
| Bova115_4007 | Predicted | Endo-1,4-beta- | GH10 | 99 | −0.01 | 1.0E+00 |
| | PUL 93 | xylanase (EC 3.2.1.8) | | | | |
| Bova115_4008 | Predicted | Xylosidase/ | GH43_1 | 99 | 0.00 | 1.0E+00 |
| | PUL 93 | arabinosidase | | | | |
| Bova115_4009 | Predicted | Xylan alpha-1,2- | GH67 | 99 | 0.01 | 1.0E+00 |
| | PUL 93 | glucuronosidase (EC | | | | |
| | | 3.2.1.131) | | | | |
| Bova115_4013 | Predicted | RNA polymerase | REG | 100 | −0.01 | 1.0E+00 |
| | PUL 94 | sigma-70 factor | | | | |
| Bova115_4014 | Predicted | Putative anti-sigma | REG | 100 | 0.03 | 1.0E+00 |
| | PUL 94 | factor | | | | |
| Bova115_4015 | Predicted | Outer membrane | Trans | 97 | −0.11 | 6.1E−01 |
| | PUL 94 | TonB-dependent | | | | |
| | | transporter, SusC | | | | |
| | | family | | | | |
| Bova115_4016 | Predicted | Cell surface glycan- | SusD | 100 | 0.06 | 1.0E+00 |
| | PUL 94 | binding lipoprotein, | | | | |
| | | SusD family | | | | |
| Bova115_4017 | Predicted | hypothetical protein | | 99 | −0.06 | 1.0E+00 |
| | PUL 94 | | | | | |
| Bova115_4032 | Predicted | Mucin-desulfating | Sulf | 99 | −0.06 | 7.4E−01 |
| | PUL 95 | sulfatase | | | | |
| Bova115_4033 | Predicted | beta-glycosyl | GH20 | 99 | 0.02 | 1.0E+00 |
| | PUL 95 | hydrolase | | | | |
| Bova115_4034 | Predicted | beta-galactosidase | GH2 | 100 | 0.01 | 1.0E+00 |
| | PUL 95 | (EC 3.2.1.23) | | | | |
| Bova115_4035 | Predicted | glycosyl hydrolase | GH29 | 100 | −0.22 | 2.1E−01 |
| | PUL 95 | family 29 (alpha-L- | | | | |
| | | fucosidase) | | | | |
| Bova115_4091 | Predicted | Cell surface glycan- | SusD | 100 | 0.01 | 1.0E+00 |
| | PUL 96 | binding lipoprotein, | | | | |
| | | SusD family | | | | |
| Bova115_4092 | Predicted | Outer membrane | Trans | 98 | −0.05 | 1.0E+00 |
| | PUL 96 | TonB-dependent | | | | |
| | | transporter, SusC | | | | |
| | | family | | | | |
| Bova115_4093 | Predicted | FIG00938541: | | 99 | −0.01 | 1.0E+00 |
| | PUL 96 | hypothetical protein | | | | |
| Bova115_4192 | Predicted | Outer membrane | Trans | 100 | 0.03 | 9.0E−01 |
| | PUL 97 | TonB-dependent | | | | |
| | | transporter, SusC | | | | |
| | | family | | | | |
| Bova115_4193 | Predicted | hypothetical protein | SusD | 100 | 0.04 | 1.0E+00 |
| | PUL 97 | | | | | |
| Bova115_4194 | Predicted | hypothetical protein | | 100 | −0.02 | 1.0E+00 |
| | PUL 97 | | | | | |
| Bova115_4195 | Predicted | hypothetical protein | | 95 | — | — |
| | PUL 97 | | | | | |
| Bova115_4196 | Predicted | hypothetical protein | | 76 | 0.00 | 1.0E+00 |
| | PUL 97 | | | | | |
| Bova115_4197 | Predicted | hypothetical protein | | 100 | −0.04 | 8.8E−01 |
| | PUL 97 | | | | | |
| Bova115_5286 | Predicted | Uncharacterized MFS- | Trans | 65 | −0.03 | 1.0E+00 |
| | PUL 103 | type transporter | | | | |
| Bova115_4252 | Predicted | RNA polymerase | REG | 99 | 0.13 | 3.9E−01 |
| | PUL 98 | ECF-type sigma factor | | | | |
| Bova115_4253 | Predicted | Putative anti-sigma | REG | 99 | 0.05 | 1.0E+00 |
| | PUL 98 | factor | | | | |
| Bova115_4254 | Predicted | Outer membrane | Trans | 100 | −0.05 | 1.0E+00 |
| | PUL 98 | TonB-dependent | | | | |
| | | transporter, SusC | | | | |
| | | family | | | | |
| Bova115_4255 | Predicted | Cell surface glycan- | SusD | 99 | 0.01 | 1.0E+00 |
| | PUL 98 | binding lipoprotein, | | | | |
| | | SusD family | | | | |
| Bova115_4256 | Predicted | hypothetical protein | | 97 | 0.02 | 1.0E+00 |
| | PUL 98 | | | | | |
| Bova115_4257 | Predicted | hypothetical protein | | 98 | −0.11 | 1.0E+00 |
| | PUL 98 | | | | | |
| Bova115_4279 | Predicted | Outer membrane | Trans | 99 | 0.04 | 9.0E−01 |
| | PUL 99 | TonB-dependent | | | | |
| | | transporter, SusC | | | | |
| | | family | | | | |
| Bova115_4280 | Predicted | Cell surface glycan- | SusD | 99 | −0.11 | 5.6E−01 |
| | PUL 99 | binding lipoprotein, | | | | |
| | | SusD family | | | | |
| Bova115_4456 | Predicted | RNA polymerase | REG | 100 | −0.02 | 1.0E+00 |
| | PUL 100 | ECF-type sigma factor | | | | |
| Bova115_4457 | Predicted | hypothetical protein | REG | 99 | 0.30 | 1.8E−01 |
| | PUL 100 | | | | | |
| Bova115_4458 | Predicted | Outer membrane | Trans | 100 | −0.03 | 1.0E+00 |
| | PUL 100 | TonB-dependent | | | | |
| | | transporter, SusC | | | | |
| | | family | | | | |
| Bova115_4459 | Predicted | Cell surface glycan- | SusD | 100 | −0.02 | 1.0E+00 |
| | PUL 100 | binding lipoprotein, | | | | |
| | | SusD family | | | | |
| Bova115_4460 | Predicted | Outer membrane | Trans | 100 | 0.09 | 1.0E+00 |
| | PUL 100 | TonB-dependent | | | | |
| | | transporter, SusC | | | | |
| | | family | | | | |
| Bova115_4461 | Predicted | Cell surface glycan- | GH16 | 100 | 0.03 | 1.0E+00 |
| | PUL 100 | binding lipoprotein, | | | | |
| | | SusD family | | | | |
| Bova115_4462 | Predicted | beta-glucosidase (EC | GH16-GH3 | 99 | 0.00 | 1.0E+00 |
| | PUL 100 | 3.2.1.21) | | | | |
| Bova115_4463 | Predicted | putative purple acid | | 100 | 0.00 | 1.0E+00 |
| | PUL 100 | phosphatase | | | | |
| Bova115_4464 | Predicted | hypothetical protein | | 100 | 0.03 | 9.0E−01 |
| | PUL 100 | | | | | |
| Bova115_4488 | Predicted | endo-beta-N- | GH | 100 | −0.01 | 1.0E+00 |
| | PUL 101 | acetylglucosaminidase | | | | |
| Bova115_4489 | Predicted | putative patatin-like | | 100 | 0.00 | 1.0E+00 |
| | PUL 101 | protein | | | | |
| Bova115_4490 | Predicted | hypothetical protein | | 99 | 0.01 | 1.0E+00 |
| | PUL 101 | | | | | |
| Bova115_4491 | Predicted | Cell surface glycan- | SusD | 100 | 0.01 | 1.0E+00 |
| | PUL 101 | binding lipoprotein, | | | | |
| | | SusD family | | | | |
| Bova115_4492 | Predicted | Outer membrane | Trans | 99 | −0.03 | 1.0E+00 |
| | PUL 101 | TonB-dependent | | | | |
| | | transporter, SusC | | | | |
| | | family | | | | |
| Bova115_4493 | Predicted | Putative anti-sigma | REG | 99 | −0.03 | 1.0E+00 |
| | PUL 101 | factor | | | | |
| Bova115_4494 | Predicted | RNA polymerase | REG | 99 | −0.02 | 1.0E+00 |
| | PUL 101 | ECF-type sigma factor | | | | |

Abbreviation
Classification (CAZymes)
CBM Carbohydrate-Binding Module
CE Carbohydrate Esterase
GH Glycoside Hydrolase
PL Polysaccharide Lyase
Abbreviation
Classification
Epi Epimerase
Int Integrase
Pept Peptidase
REG Regulators
ROK Regulator/ORF/Kinase Family
Sulf Sulfatase
SusD PUL marker gene (SusD-like outer member binding protein)
Trans Transporters (e.g. SusC-like TonB-dependent transporters)

TABLE S8A
Composition of the snack food prototypes.

| | Extruded orange | | Extruded pea | | Raw pea fiber | |
|---|---|---|---|---|---|---|
| | fiber bar | | fiber bar | | rotary biscuit | |
| | | Per | | Per | | Per |
| | Per | portion | Per | portion | Per | portion |
| | 100 g | (35 g) | 100 g | (35 g) | 100 g | (35 g) |
| Energy (kcal) | 351.0 | 123.0 | 370.0 | 130.0 | 437.0 | 153.0 |
| Protein (g) | 4.0 | 1.4 | 9.0 | 3.2 | 10.0 | 3.5 |
| Lipid (g) | 9.0 | 3.2 | 10.0 | 3.5 | 18.0 | 6.3 |
| Sugars (g) | 23.0 | 8.1 | 20.0 | 7.0 | 17.0 | 6.0 |
| Starch (g) | 12.0 | 4.2 | 16.0 | 5.6 | 31.0 | 10.9 |
| Total dietary | 29.0 | 10.2 | 23.0 | 8.1 | 19.0 | 6.7 |
| fiber (g) | | | | | | |

TABLE S8B
Ages and BMIs of participants plus levels of N-methylserotonin and serotonin
in their fecal samples collected at the end of study weeks 1, 3 and 5.

| | | | | | Enrolled | | N-methylserotonin |
|---|---|---|---|---|---|---|---|
| | | | | | in | Enrolled | (μg/g) |
| Partic- | | | | | orange | in pea | Orange |
| ipant | | Race/ | Age | BMI | fiber | fiber | fiber snacks |
| ID | Gender | ethnicity | (years) | (kg/m²) | study | study | Week 1 |
| TP01-01 | Female | White | 41 | 38 | Yes | Yes | 4.8 |
| | | (Caucasian) | | | | | |
| TP01-02 | Female | White | 41 | 30 | Yes | Yes | 3 |
| | | (Caucasian) | | | | | |
| TP02-01 | Female | White | 38 | 36 | Yes | Yes | 0 |
| | | (Caucasian) | | | | | |
| TP02-02 | Female | White | 38 | 25 | Yes | Yes | 0 |
| | | (Caucasian) | | | | | |
| TP03-01 | Female | Black or | 37 | 46 | Yes | Yes | 0 |
| | | African | | | | | |
| | | American | | | | | |
| TP03-02 | Female | Black or | 37 | 47 | Yes | Yes | 0 |
| | | African | | | | | |
| | | American | | | | | |
| TP04-01 | Female | White | 33 | 38 | Yes | Yes | 0 |
| | | (Caucasian) | | | | | |
| TP04-02 | Female | White | 33 | 38 | Yes | Yes | 0 |
| | | (Caucasian) | | | | | |
| TP05-01 | Female | White | 32 | 45 | Yes | Yes | 0 |
| | | (Caucasian) | | | | | |
| TP05-02 | Female | White | 32 | 34 | Yes | Yes | 0.9 |
| | | (Caucasian) | | | | | |
| TP06-01 | Female | White | 37 | 41 | Yes | Yes | 0 |
| | | (Caucasian) | | | | | |
| TP06-02 | Female | White | 37 | 29 | Yes | Yes | 0 |
| | | (Caucasian) | | | | | |
| TP07-01 | Female | White | 37 | 24 | Yes | Yes | 0 |
| | | (Caucasian) | | | | | |
| TP07-02 | Female | White | 37 | 34 | Yes | Yes | 0 |
| | | (Caucasian) | | | | | |
| TP08-01 | Female | White | 42 | 21 | Yes | Yes | 0 |
| | | (Caucasian) | | | | | |
| TP08-02 | Female | White | 42 | 31 | Yes | Yes | 0 |
| | | (Caucasian) | | | | | |
| TP09-01 | Female | White | 38 | 27 | Yes | Yes | 0 |
| | | (Caucasian) | | | | | |
| TP09-02 | Female | White | 38 | 39 | Yes | Yes | 0 |
| | | (Caucasian) | | | | | |
| TP10-01 | Female | White | 36 | 31 | Yes | No | 5 |
| | | (Caucasian) | | | | | |
| TP10-02 | Female | White | 36 | 36 | Yes | No | 0 |
| | | (Caucasian) | | | | | |
| TP11-01 | Female | White | 33 | 46 | Yes | No | 10 |
| | | (Caucasian) | | | | | |
| TP11-02 | Female | White | 33 | 53 | Yes | No | 0 |
| | | (Caucasian) | | | | | |
| TP12-01 | Female | White | 37 | 42 | Yes | No | 0 |
| | | (Caucasian) | | | | | |
| TP12-02 | Female | White | 37 | 35 | Yes | No | 0 |
| | | (Caucasian) | | | | | |

| Partic- | N-methylserotonin (μg/g) | | | | | Serotonin (μg/g) | | |
|---|---|---|---|---|---|---|---|---|
| ipant | Orange fiber snacks | | Pea fiber snacks | | | Orange fiber snacks | | |
| ID | Week 3 | Week 5 | Week 1 | Week 3 | Week 5 | Week 1 | Week 3 | Week 5 |
| TP01-01 | 9 | 23.7 | 17 | 0 | 12 | 1.6 | 0.5 | 1.2 |
| TP01-02 | 9.6 | 22.2 | 42 | 0 | 0 | 1.3 | 0.2 | 0.2 |
| TP02-01 | 10.7 | 7.9 | 0 | 0 | 0 | 1.5 | 1.6 | 0.5 |
| TP02-02 | 11.8 | 25.8 | 0 | 0 | 0 | 2.7 | 1.2 | 0 |
| TP03-01 | 7.4 | 8.1 | 0 | 0 | 0 | 1 | 1.3 | 0.9 |
| TP03-02 | 11.9 | 20.2 | 0 | 0 | 0 | 0.5 | 0.4 | 0.5 |
| TP04-01 | 4.2 | 13.7 | 0 | 0 | 0 | 0.8 | 0.9 | 1.2 |
| TP04-02 | 9.1 | 10.7 | 0 | 0 | 0 | 1.6 | 1.8 | 0.9 |
| TP05-01 | 6.3 | 9.4 | 0 | 0 | 0 | 1.6 | 2.5 | 1.1 |
| TP05-02 | 7.4 | 17.5 | 1 | 0 | 1.3 | 2.9 | 1.5 | 2.1 |
| TP06-01 | 3.6 | 2.3 | 0 | 0 | 0 | 2 | 0.8 | 0.9 |
| TP06-02 | 3.3 | 6.9 | 0 | 0 | 0 | 0.7 | 1 | 0.4 |
| TP07-01 | 6.4 | 21.6 | 2 | 5 | 0 | 1.1 | 2.7 | 2.6 |
| TP07-02 | 6 | 17.4 | 0 | 0 | 0 | 0.7 | 1.5 | 0.6 |
| TP08-01 | 15.8 | 21.4 | 0 | 0 | 0 | 1.7 | 0.5 | 0.7 |
| TP08-02 | 6.2 | 21 | 0 | 0 | 0 | 1.3 | 1.5 | 0.5 |
| TP09-01 | 3 | 4.4 | 0 | 0 | 0 | 0.6 | 0.5 | 1.4 |
| TP09-02 | 1.1 | 3.5 | 0 | 0 | 0 | 2.5 | 0.8 | 0.5 |
| TP10-01 | 20.6 | 21.4 | | | | 2.5 | 1.1 | 3.9 |
| TP10-02 | 7.8 | 3.2 | | | | 0.1 | 1 | 2.1 |
| TP11-01 | 10.5 | 10.9 | | | | 1 | 1 | 0.5 |
| TP11-02 | 0 | 10 | | | | 0.3 | 0.2 | 1.7 |
| TP12-01 | 4.1 | 16.7 | | | | 0.8 | 0.6 | 0.8 |
| TP12-02 | 10.2 | 12.3 | | | | 0.7 | 7.5 | 0.5 |

Note that the four individuals who provided the seven positive fecal samples in the pea fiber study also participated in the subsequent orange fiber study; four of these seven samples tested positive during the pre-treatment phase of the pea fiber study.
The level documented for subject TP-01-01 when consuming 3 pea fiber snacks/day was 50% of the level when ingesting three orange fiber snacks/day. Our dietary histories were not sufficiently granular to quantify consumption of citrus-containing foods.

TABLE S8C
ASVs with statistically significant log₂-fold changes in relative abundances in the fecal microbiota of participants
between the pre-intervention and 5-week timepoints of the pea and orange fiber snack studies.

| | | | | | Mean Log2 Fold Change | | FDR-adjusted (q-value) | |
|---|---|---|---|---|---|---|---|---|
| | Mean (log2) ± | | | | Pea | Orange | Pea | Orange |
| | Standard Deviation | | | | Fiber, | Fiber, | Fiber, | Fiber, |
| | Pea Fiber | | Orange Fiber | | Week 5 | Week 5 | Week 5 | Week 5 |
| | Pre- | | Pre- | | vs Pre- | vs Pre- | vs Pre- | vs Pre- |
| Taxa | intervention | Week 5 | intervention | Week 5 | intervention | intervention | intervention | intervention |
| Monoglobus | 0.3 ± 0.2 | 1.4 ± 1.1 | 0.4 ± 0.3 | 0.3 ± 0.2 | 1.015 | −0.088 | 0.002 | 0.698 |
| pectinilyticus | | | | | | | | |
| ASV71 | | | | | | | | |
| Herbinix | 0.0 ± 0.1 | 0.9 ± 2.0 | 0.1 ± 0.1 | 0.1 ± 0.3 | 0.662 | 0.002 | 0.047 | 0.101 |
| luporum | | | | | | | | |
| ASV128 | | | | | | | | |
| Lachnospiraceae | 0.1 ± 0.1 | 0.4 ± 0.5 | 0.3 ± 0.5 | 0.1 ± 0.3 | 0.467 | −0.199 | 0.024 | 0.028 |
| NK4A136 group | | | | | | | | |
| ASV148 | | | | | | | | |
| Ruminococcus | 0.2 ± 0.4 | 0.7 ± 1.5 | 0.4 ± 0.7 | 0.5 ± 1.3 | 0.436 | 0.088 | 0.051 | 0.455 |
| callidus ASV112 | | | | | | | | |
| Lachnospiraceae | 0.1 ± 0.1 | 0.4 ± 0.4 | 0.2 ± 0.2 | 0.4 ± 0.6 | 0.395 | 0.281 | 0.043 | 0.137 |
| UCG-001 ASV179 | | | | | | | | |
| Faecalibacterium | 1.9 ± 2.0 | 2.7 ± 2.7 | 1.9 ± 2.6 | 3.1 ± 3.5 | 0.385 | 0.538 | 0.008 | 0.017 |
| prausnitzii ASV19 | | | | | | | | |
| Bifidobacterium sp. | 0.4 ± 0.5 | 0.7 ± 0.7 | 0.5 ± 0.9 | 0.7 ± 0.9 | 0.384 | 0.251 | 0.049 | 0.196 |
| ASV59 | | | | | | | | |
| Christensenellaceae | 0.1 ± 0.2 | 0.6 ± 1.0 | 0.1 ± 0.3 | 0.1 ± 0.3 | 0.359 | 0.086 | 0.023 | 0.124 |
| R-7 group ASV150 | | | | | | | | |
| Ruminiclostridium sp. | 0.1 ± 0.2 | 0.4 ± 0.8 | 0.5 ± 1.2 | 1.2 ± 3.3 | 0.275 | 0.150 | 0.175 | 0.953 |
| ASV68 | | | | | | | | |
| Dialister invisus | 0.3 ± 0.7 | 0.5 ± 0.9 | 1.1 ± 2.0 | 1.4 ± 3.6 | 0.217 | 0.096 | 0.189 | 0.295 |
| ASV67 | | | | | | | | |
| Marvinbryantia sp. | 0.0 ± 0.1 | 0.1 ± 0.2 | 0.0 ± 0.0 | 0.0 ± 0.0 | 0.182 | 0.012 | 0.028 | 0.865 |
| ASV212 | | | | | | | | |
| Ruminococcus sp. | 0.6 ± 0.6 | 0.4 ± 0.4 | 0.5 ± 0.7 | 0.5 ± 0.6 | −0.249 | −0.101 | 0.069 | 0.769 |
| ASV23 | | | | | | | | |
| Roseburia sp. | 5.6 ± 5.9 | 3.8 ± 3.2 | 5.5 ± 5.8 | 3.1 ± 3.6 | −0.258 | −0.659 | 0.661 | 0.110 |
| ASV11 | | | | | | | | |
| Ruminococcus faecis | 1.0 ± 1.2 | 0.6 ± 0.9 | 0.7 ± 0.8 | 0.6 ± 0.9 | −0.477 | −0.292 | 0.041 | 0.150 |
| ASV38 | | | | | | | | |
| Lachnospira | 0.2 ± 0.3 | 0.3 ± 0.4 | 0.3 ± 0.4 | 1.3 ± 2.0 | 0.203 | 0.731 | 0.204 | 0.012 |
| pectinoschiza | | | | | | | | |
| ASV87 | | | | | | | | |
| Lachnospira | 0.1 ± 0.1 | 0.2 ± 0.3 | 0.1 ± 0.1 | 0.7 ± 0.9 | 0.093 | 0.705 | 0.720 | 0.003 |
| pectinoschiza | | | | | | | | |
| ASV167 | | | | | | | | |
| Lachnoclostridium sp. | 0.3 ± 0.4 | 0.4 ± 0.9 | 0.5 ± 0.8 | 3.2 ± 7.7 | 0.068 | 0.651 | 0.241 | 0.013 |
| ASV54 | | | | | | | | |
| [Eubacterium] | 0.2 ± 0.3 | 0.4 ± 0.5 | 0.3 ± 0.6 | 0.9 ± 1.3 | 0.236 | 0.555 | 0.036 | 0.007 |
| eligens ASV86 | | | | | | | | |
| Lachnospira sp. | 0.1 ± 0.2 | 0.4 ± 0.7 | 0.2 ± 0.4 | 0.8 ± 1.5 | 0.253 | 0.438 | 0.399 | 0.075 |
| ASV111 | | | | | | | | |
| Ruminococcus | 0.7 ± 1.0 | 1.1 ± 1.3 | 0.8 ± 1.3 | 1.7 ± 2.6 | 0.384 | 0.385 | 0.430 | 0.154 |
| bicirculans ASV28 | | | | | | | | |
| Alistipes sp. | 0.2 ± 0.4 | 0.2 ± 0.4 | 0.2 ± 0.2 | 0.1 ± 0.4 | −0.101 | −0.063 | 0.753 | 0.072 |
| ASV64 | | | | | | | | |
| Ruminococcus sp. | 0.2 ± 0.3 | 0.1 ± 0.2 | 0.1 ± 0.2 | 0.0 ± 0.1 | −0.219 | −0.138 | 0.280 | 0.050 |
| ASV157 | | | | | | | | |
| Clostridium_III sp. | 0.1 ± 0.1 | 0.2 ± 0.3 | 0.1 ± 0.1 | 0.0 ± 0.0 | 0.220 | −0.149 | 0.714 | 0.057 |
| ASV164 | | | | | | | | |
| Collinsella | 0.4 ± 0.4 | 0.3 ± 0.2 | 0.4 ± 0.5 | 0.2 ± 0.2 | −0.234 | −0.297 | 0.453 | 0.026 |
| aerofaciens | | | | | | | | |
| ASV47 | | | | | | | | |
| Ruminococcus | 0.4 ± 0.4 | 0.4 ± 0.8 | 0.7 ± 1.3 | 0.5 ± 1.4 | −0.166 | −0.303 | 0.957 | 0.004 |
| torques ASV27 | | | | | | | | |
| Bacteroides | 0.5 ± 1.0 | 0.3 ± 0.5 | 0.5 ± 0.9 | 0.2 ± 0.3 | −0.189 | −0.355 | 0.250 | 0.079 |
| massiliensis | | | | | | | | |
| ASV17 | | | | | | | | |
| Blautia glucerasea | 1.3 ± 5.1 | 1.1 ± 4.6 | 0.8 ± 2.6 | 0.1 ± 0.5 | −0.166 | −0.366 | 0.137 | 0.026 |
| ASV10 | | | | | | | | |
| Roseburia | 0.5 ± 0.6 | 0.2 ± 0.3 | 1.1 ± 1.3 | 0.2 ± 0.2 | −0.337 | −0.835 | 0.110 | 0.002 |
| inulinivorans | | | | | | | | |
| ASV74 | | | | | | | | |
| Ruminococcus | 3.5 ± 3.4 | 2.1 ± 2.3 | 3.1 ± 2.8 | 1.1 ± 1.6 | −0.632 | −1.066 | 0.056 | 0.002 |
| bromii ASV15 | | | | | | | | |

TABLE S8D
Week 1. CAZyme (GH and PL) gene representation in the fecal microbiomes of participants
enrolled in open-label, single group assignment study consuming orange fiber snacks.

| | | Participant ID | | | | | | | |
|---|---|---|---|---|---|---|---|---|---|
| | | TP01.01 | TP01.02 | TP02.01 | TP02.02 | TP03.01 | TP03.02 | TP04.01 | TP04.02 |
| | | Timepoint | | | | | | | |
| | | Week 1 | Week 1 | Week 1 | Week 1 | Week 1 | Week 1 | Week 1 | Week 1 |
| | | Diet | | | | | | | |
| | | Free | Free | Free | Free | Free | Free | Free | Free |
| | | Diet | Diet | Diet | Diet | Diet | Diet | Diet | Diet |
| Read | GH1 | 549.92 | 112.53 | 238.14 | 215.22 | 258.51 | 218.96 | 170.19 | 379.93 |
| counts | GH10 | 198.18 | 59.34 | 143.26 | 67.27 | 68.42 | 48.37 | 75.50 | 65.26 |
| of | GH101 | 7.60 | 5.89 | 12.59 | 6.09 | 0.00 | 14.97 | 0.00 | 23.09 |
| CAZyme | GH102 | 0.00 | 0.00 | 0.00 | 0.00 | 0.00 | 0.00 | 0.00 | 0.00 |
| genes | GH103 | 0.00 | 3.48 | 0.00 | 3.06 | 0.00 | 0.00 | 0.00 | 0.00 |
| (TPM) | GH105 | 212.04 | 490.43 | 1069.09 | 334.32 | 382.14 | 751.62 | 613.68 | 314.27 |
| normal- | GH106 | 106.12 | 130.39 | 304.01 | 142.97 | 133.09 | 246.71 | 122.46 | 97.58 |
| ized | GH108 | 72.52 | 69.10 | 71.71 | 55.26 | 55.35 | 68.17 | 73.38 | 35.84 |
| | GH109 | 42.43 | 216.81 | 383.97 | 170.92 | 160.15 | 468.86 | 252.34 | 159.62 |
| | GH110 | 19.04 | 100.07 | 194.26 | 143.48 | 66.84 | 138.09 | 71.11 | 54.99 |
| | GH111 | 0.00 | 0.00 | 0.00 | 0.00 | 0.00 | 0.00 | 0.00 | 0.00 |
| | GH112 | 135.74 | 65.47 | 112.18 | 60.90 | 129.91 | 95.95 | 78.10 | 162.52 |
| | GH113 | 15.36 | 4.23 | 0.00 | 19.90 | 18.47 | 0.00 | 39.02 | 14.52 |
| | GH114 | 11.99 | 8.80 | 35.33 | 0.00 | 0.00 | 0.00 | 11.40 | 0.00 |
| | GH115 | 200.12 | 99.99 | 247.67 | 61.72 | 137.49 | 161.06 | 134.01 | 82.47 |
| | GH116 | 4.26 | 37.45 | 55.91 | 34.05 | 34.83 | 87.06 | 156.41 | 2.49 |
| | GH117 | 9.66 | 33.93 | 0.00 | 0.00 | 33.57 | 17.61 | 75.55 | 4.63 |
| | GH120 | 53.17 | 13.24 | 35.79 | 22.28 | 51.83 | 11.02 | 58.49 | 154.78 |
| | GH121 | 0.00 | 9.22 | 0.00 | 14.80 | 0.00 | 0.00 | 0.00 | 18.68 |
| | GH123 | 21.10 | 85.14 | 119.80 | 78.86 | 39.67 | 169.27 | 58.04 | 45.26 |
| | GH125 | 103.87 | 196.36 | 266.99 | 237.06 | 108.84 | 203.89 | 283.92 | 23.44 |
| | GH126 | 0.00 | 0.00 | 0.00 | 6.46 | 0.00 | 0.00 | 0.00 | 0.00 |
| | GH127 | 376.67 | 162.37 | 334.27 | 164.35 | 228.68 | 193.64 | 216.85 | 216.77 |
| | GH128 | 0.00 | 0.00 | 0.00 | 0.00 | 0.00 | 0.00 | 0.00 | 25.88 |
| | GH129 | 30.03 | 16.29 | 3.26 | 6.16 | 9.29 | 7.68 | 0.00 | 32.07 |
| | GH13 | 608.10 | 883.67 | 461.29 | 495.54 | 581.76 | 576.81 | 598.31 | 531.85 |
| | GH13 11 | 171.41 | 135.31 | 153.14 | 134.00 | 188.05 | 74.79 | 76.75 | 279.59 |
| | GH13 12 | 0.00 | 0.00 | 0.00 | 0.00 | 0.00 | 0.00 | 0.00 | 0.00 |
| | GH13 14 | 205.30 | 424.36 | 202.44 | 217.65 | 267.19 | 197.47 | 173.80 | 175.28 |
| | GH13 18 | 72.57 | 27.68 | 18.71 | 40.64 | 78.65 | 34.63 | 20.41 | 77.41 |
| | GH13 19 | 0.00 | 110.56 | 0.00 | 30.42 | 29.92 | 6.92 | 22.38 | 7.95 |
| | GH13 2 | 0.00 | 0.00 | 0.00 | 0.00 | 0.00 | 0.00 | 0.00 | 0.00 |
| | GH13 20 | 144.99 | 42.12 | 65.66 | 70.33 | 182.76 | 99.03 | 31.93 | 152.97 |
| | GH13 28 | 0.00 | 257.22 | 0.00 | 74.27 | 69.14 | 17.17 | 33.39 | 37.45 |
| | GH13 29 | 24.80 | 0.00 | 29.85 | 6.48 | 21.04 | 13.52 | 0.00 | 8.40 |
| | GH13 3 | 6.11 | 6.30 | 0.00 | 10.57 | 0.00 | 6.32 | 0.00 | 25.23 |
| | GH13 30 | 7.19 | 10.20 | 0.00 | 11.54 | 0.00 | 6.35 | 0.00 | 30.69 |
| | GH13 31 | 252.89 | 85.78 | 185.80 | 194.20 | 283.30 | 85.57 | 136.86 | 391.07 |
| | GH13 32 | 0.00 | 6.71 | 0.00 | 8.40 | 0.00 | 0.00 | 0.00 | 3.74 |
| | GH13 36 | 61.44 | 134.72 | 30.29 | 60.08 | 125.89 | 22.83 | 47.07 | 85.14 |
| | GH13 37 | 0.00 | 28.96 | 0.00 | 0.00 | 0.00 | 0.00 | 0.00 | 0.00 |
| | GH13 38 | 103.59 | 70.92 | 119.76 | 60.45 | 71.52 | 119.08 | 87.60 | 47.87 |
| | GH13 39 | 46.34 | 182.01 | 8.90 | 116.39 | 61.13 | 62.76 | 64.31 | 67.08 |
| | GH13 4 | 49.76 | 16.08 | 69.84 | 34.21 | 42.13 | 30.33 | 83.14 | 75.20 |
| | GH13 41 | 59.31 | 5.24 | 28.61 | 37.28 | 69.47 | 0.00 | 13.04 | 69.97 |
| | GH13 42 | 0.00 | 0.00 | 0.00 | 0.00 | 0.00 | 0.00 | 0.00 | 0.00 |
| | GH13 5 | 9.76 | 29.85 | 20.79 | 18.83 | 0.00 | 5.87 | 15.76 | 0.00 |
| | GH13 8 | 100.30 | 87.80 | 113.00 | 85.84 | 68.82 | 98.21 | 86.94 | 49.97 |
| | GH13 9 | 250.21 | 338.20 | 160.11 | 271.97 | 302.49 | 212.41 | 254.43 | 386.39 |
| | GH130 | 311.89 | 267.05 | 480.12 | 235.08 | 221.34 | 332.24 | 425.29 | 131.99 |
| | GH133 | 128.98 | 78.44 | 180.83 | 88.71 | 106.51 | 128.04 | 121.51 | 130.71 |
| | GH136 | 41.70 | 52.36 | 134.64 | 38.58 | 77.61 | 92.00 | 69.87 | 88.85 |
| | GH137 | 92.16 | 17.88 | 45.80 | 14.28 | 18.73 | 28.39 | 24.85 | 29.53 |
| | GH138 | 97.52 | 18.84 | 35.35 | 14.07 | 21.36 | 27.53 | 38.07 | 29.12 |
| | GH139 | 0.00 | 31.46 | 77.41 | 21.36 | 24.65 | 86.67 | 30.50 | 29.06 |
| | GH140 | 96.32 | 50.50 | 146.24 | 46.16 | 43.53 | 144.55 | 77.73 | 69.54 |
| | GH141 | 104.80 | 64.09 | 175.59 | 30.89 | 54.99 | 149.76 | 72.25 | 54.83 |
| | GH142 | 0.00 | 27.01 | 90.09 | 29.67 | 26.04 | 63.49 | 23.97 | 28.56 |
| | GH143 | 0.00 | 27.01 | 84.57 | 24.16 | 21.30 | 63.49 | 23.97 | 36.97 |
| | GH144 | 80.30 | 111.44 | 184.30 | 113.05 | 111.51 | 68.52 | 211.57 | 14.68 |
| | GH145 | 8.99 | 69.63 | 140.40 | 57.63 | 51.91 | 92.99 | 92.28 | 39.93 |
| | GH146 | 22.64 | 184.77 | 241.62 | 103.30 | 160.14 | 207.80 | 176.00 | 104.77 |
| | GH147 | 0.00 | 7.18 | 10.60 | 9.32 | 31.95 | 5.14 | 17.14 | 0.00 |
| | GH148 | 0.00 | 0.00 | 0.00 | 7.69 | 6.52 | 0.00 | 5.99 | 7.76 |
| | GH15 | 0.00 | 28.43 | 86.36 | 65.65 | 16.15 | 111.27 | 39.31 | 25.09 |
| | GH151 | 0.00 | 7.51 | 24.15 | 2.97 | 0.00 | 0.00 | 0.00 | 14.35 |
| | GH153 | 0.00 | 0.00 | 0.00 | 0.00 | 0.00 | 0.00 | 0.00 | 0.00 |
| | GH154 | 20.08 | 122.17 | 358.64 | 110.37 | 160.70 | 223.61 | 189.94 | 73.42 |
| | GH155 | 0.00 | 34.89 | 450.67 | 101.03 | 57.87 | 28.05 | 103.04 | 2.36 |
| | GH156 | 0.00 | 0.00 | 0.00 | 7.81 | 0.00 | 3.99 | 0.00 | 0.00 |
| | GH157 | 0.00 | 0.00 | 0.00 | 0.00 | 0.00 | 0.00 | 0.00 | 0.00 |
| | GH158 | 0.00 | 29.09 | 54.78 | 40.46 | 16.97 | 15.75 | 48.09 | 0.00 |
| | GH159 | 0.00 | 6.62 | 0.00 | 9.44 | 5.78 | 0.00 | 0.00 | 0.00 |
| | GH16 | 184.95 | 183.63 | 306.22 | 194.12 | 210.01 | 225.93 | 290.98 | 223.56 |
| | GH161 | 0.00 | 0.00 | 0.00 | 10.63 | 9.08 | 0.00 | 0.00 | 0.00 |
| | GH17 | 0.00 | 0.00 | 0.00 | 0.00 | 0.00 | 0.00 | 0.00 | 0.00 |
| | GH18 | 137.25 | 296.33 | 885.55 | 613.97 | 271.76 | 314.45 | 481.85 | 256.94 |
| | GH19 | 0.00 | 0.00 | 6.12 | 0.00 | 0.00 | 0.00 | 0.00 | 0.00 |
| | GH2 | 1664.79 | 1870.64 | 3082.83 | 1808.06 | 1967.87 | 2793.17 | 2421.50 | 1340.20 |
| | GH20 | 221.84 | 747.91 | 1149.66 | 771.02 | 514.92 | 1298.64 | 851.82 | 447.53 |
| | GH23 | 533.84 | 557.85 | 602.48 | 739.27 | 490.76 | 517.48 | 507.08 | 627.28 |
| | GH24 | 48.03 | 265.32 | 469.93 | 193.84 | 290.57 | 337.92 | 238.75 | 130.52 |
| | GH25 | 665.03 | 559.92 | 308.05 | 469.79 | 617.48 | 389.75 | 444.47 | 414.83 |
| | GH26 | 159.60 | 143.41 | 129.64 | 175.11 | 110.70 | 78.03 | 244.93 | 0.00 |
| | GH27 | 100.10 | 150.21 | 215.34 | 159.60 | 109.42 | 127.94 | 205.77 | 103.71 |
| | GH28 | 605.35 | 645.99 | 1253.61 | 396.05 | 777.78 | 1021.96 | 716.67 | 539.89 |
| | GH29 | 114.56 | 426.55 | 908.95 | 378.42 | 535.64 | 1241.55 | 554.53 | 496.72 |
| | GH3 | 1222.65 | 1438.74 | 1801.69 | 1309.76 | 1092.28 | 1337.84 | 1759.39 | 937.86 |
| | GH30 | 0.00 | 0.00 | 0.00 | 0.00 | 0.00 | 6.45 | 0.00 | 0.00 |
| | GH30 1 | 0.00 | 14.93 | 20.72 | 2.90 | 0.00 | 7.13 | 0.00 | 26.13 |
| | GH30 2 | 6.29 | 31.26 | 3.70 | 12.62 | 0.00 | 29.87 | 15.52 | 34.03 |
| | GH30 3 | 76.54 | 42.59 | 69.83 | 46.17 | 17.74 | 22.39 | 55.79 | 0.00 |
| | GH30 4 | 92.57 | 94.09 | 160.26 | 70.61 | 22.68 | 155.88 | 133.10 | 65.66 |
| | GH30 5 | 0.00 | 0.00 | 0.00 | 0.00 | 0.00 | 0.00 | 0.00 | 0.00 |
| | GH30 6 | 10.26 | 21.68 | 77.53 | 18.49 | 10.29 | 82.13 | 37.99 | 39.86 |
| | GH30 8 | 5.59 | 0.00 | 17.51 | 0.00 | 0.00 | 0.00 | 5.14 | 0.00 |
| | GH31 | 478.47 | 736.75 | 842.85 | 614.26 | 579.24 | 643.39 | 660.22 | 619.56 |
| | GH32 | 510.06 | 265.79 | 468.27 | 388.12 | 542.52 | 319.04 | 461.40 | 526.94 |
| | GH33 | 202.26 | 264.21 | 271.93 | 232.67 | 207.35 | 337.37 | 196.63 | 115.88 |
| | GH35 | 97.63 | 165.37 | 257.64 | 112.64 | 168.61 | 163.37 | 180.97 | 44.63 |
| | GH36 | 375.44 | 334.84 | 615.34 | 427.05 | 382.37 | 385.03 | 432.91 | 341.95 |
| | GH37 | 0.00 | 10.23 | 0.00 | 0.00 | 0.00 | 0.00 | 0.00 | 0.00 |
| | GH38 | 31.36 | 108.58 | 220.74 | 171.40 | 112.77 | 90.12 | 260.41 | 47.61 |
| | GH39 | 58.98 | 5.61 | 32.59 | 3.01 | 24.12 | 44.45 | 33.42 | 163.36 |
| | GH4 | 92.83 | 15.15 | 105.72 | 108.37 | 26.86 | 56.45 | 24.65 | 161.40 |
| | GH42 | 158.42 | 121.49 | 271.37 | 199.94 | 217.91 | 138.16 | 211.83 | 287.05 |
| | GH43 | 0.00 | 5.51 | 130.81 | 77.06 | 45.92 | 73.46 | 120.72 | 12.60 |
| | GH43 1 | 96.87 | 26.75 | 72.05 | 23.62 | 43.07 | 34.26 | 40.79 | 31.07 |
| | GH43 10 | 417.83 | 243.45 | 398.15 | 138.71 | 327.04 | 213.02 | 232.26 | 240.45 |
| | GH43 11 | 48.60 | 33.96 | 42.85 | 22.44 | 17.80 | 6.68 | 41.27 | 79.38 |
| | GH43 12 | 150.27 | 104.77 | 239.46 | 149.99 | 156.79 | 169.43 | 206.21 | 151.19 |
| | GH43 16 | 0.00 | 39.84 | 35.29 | 37.47 | 45.37 | 18.20 | 57.48 | 28.78 |
| | GH43 17 | 11.10 | 43.00 | 53.52 | 16.43 | 38.36 | 85.19 | 36.01 | 38.47 |
| | GH43 18 | 7.28 | 45.15 | 192.34 | 50.76 | 62.67 | 140.79 | 110.41 | 63.96 |
| | GH43 19 | 88.70 | 106.10 | 223.30 | 85.80 | 80.16 | 164.48 | 107.22 | 65.67 |
| | GH43 2 | 93.34 | 13.83 | 19.79 | 16.20 | 25.44 | 6.30 | 20.36 | 0.00 |
| | GH43 22 | 29.45 | 15.59 | 14.70 | 0.00 | 0.00 | 9.06 | 6.55 | 51.58 |
| | GH43 24 | 6.75 | 193.78 | 310.45 | 140.04 | 150.71 | 194.60 | 154.08 | 66.90 |
| | GH43 26 | 92.43 | 10.43 | 73.34 | 68.94 | 56.13 | 0.00 | 48.20 | 86.45 |
| | GH43 27 | 5.02 | 6.66 | 0.00 | 12.93 | 7.13 | 2.68 | 11.19 | 18.48 |
| | GH43 28 | 24.21 | 67.30 | 77.44 | 39.98 | 49.85 | 79.09 | 119.40 | 0.00 |
| | GH43 29 | 198.71 | 48.33 | 110.78 | 49.53 | 40.75 | 53.69 | 61.83 | 48.58 |
| | GH43 3 | 7.20 | 73.75 | 146.18 | 75.69 | 29.16 | 52.36 | 108.81 | 26.39 |
| | GH43 30 | 0.00 | 0.00 | 0.00 | 7.35 | 0.00 | 0.00 | 0.00 | 0.00 |
| | GH43 31 | 6.96 | 143.53 | 210.22 | 93.49 | 59.67 | 131.44 | 151.09 | 60.68 |
| | GH43 32 | 0.00 | 6.87 | 0.00 | 0.00 | 6.87 | 0.00 | 3.87 | 0.00 |
| | GH43 33 | 4.26 | 31.80 | 37.52 | 42.38 | 21.16 | 22.27 | 49.87 | 4.42 |
| | GH43 34 | 92.51 | 107.84 | 381.59 | 147.24 | 112.64 | 209.86 | 213.70 | 122.05 |
| | GH43 35 | 25.41 | 82.73 | 73.50 | 51.74 | 61.04 | 38.12 | 106.16 | 49.12 |
| | GH43 37 | 0.00 | 0.00 | 0.00 | 0.00 | 3.16 | 0.00 | 0.00 | 2.29 |
| | GH43 4 | 164.59 | 59.84 | 155.47 | 91.98 | 98.16 | 151.55 | 91.88 | 133.28 |
| | GH43 5 | 101.26 | 0.00 | 0.00 | 0.00 | 0.00 | 0.00 | 0.00 | 0.00 |
| | GH43 7 | 80.21 | 4.66 | 0.00 | 0.00 | 17.82 | 0.00 | 0.00 | 0.00 |
| | GH43 8 | 0.00 | 0.00 | 20.24 | 15.97 | 22.77 | 5.71 | 0.00 | 0.00 |
| | GH43 9 | 6.70 | 41.55 | 114.65 | 37.89 | 41.09 | 118.06 | 40.91 | 29.13 |
| | GH44 | 0.00 | 0.00 | 0.00 | 0.00 | 0.00 | 0.00 | 8.78 | 0.00 |
| | GH48 | 6.14 | 3.29 | 0.00 | 0.00 | 0.00 | 0.00 | 0.00 | 0.00 |
| | GH5 | 27.24 | 21.05 | 13.61 | 74.84 | 5.20 | 4.89 | 20.37 | 20.26 |
| | GH5 1 | 4.34 | 6.72 | 0.00 | 4.71 | 9.71 | 0.00 | 6.10 | 0.00 |
| | GH5 13 | 0.00 | 0.00 | 41.95 | 29.54 | 0.00 | 16.32 | 38.12 | 0.00 |
| | GH5 18 | 0.00 | 0.00 | 0.00 | 0.00 | 0.00 | 0.00 | 0.00 | 13.97 |
| | GH5 2 | 46.96 | 61.52 | 95.92 | 80.37 | 103.35 | 57.76 | 119.03 | 60.39 |
| | GH5 21 | 80.99 | 13.13 | 5.21 | 9.25 | 0.00 | 7.34 | 18.08 | 0.00 |
| | GH5 22 | 0.00 | 0.00 | 0.00 | 0.00 | 0.00 | 0.00 | 0.00 | 0.00 |
| | GH5 25 | 0.00 | 0.00 | 0.00 | 0.00 | 0.00 | 19.75 | 0.00 | 0.00 |
| | GH5 26 | 0.00 | 0.00 | 0.00 | 0.00 | 0.00 | 0.00 | 51.79 | 0.00 |
| | GH5 27 | 0.00 | 0.00 | 0.00 | 0.00 | 0.00 | 0.00 | 0.00 | 0.00 |
| | GH5 37 | 5.86 | 10.13 | 0.00 | 0.00 | 15.07 | 0.00 | 26.92 | 0.00 |
| | GH5 38 | 0.00 | 0.00 | 18.09 | 8.13 | 0.00 | 4.89 | 0.00 | 0.00 |
| | GH5 4 | 180.97 | 171.27 | 123.65 | 130.75 | 101.54 | 47.39 | 171.92 | 0.00 |
| | GH5 44 | 11.91 | 15.50 | 3.25 | 22.86 | 0.00 | 7.98 | 0.00 | 34.60 |
| | GH5 46 | 0.00 | 45.03 | 39.30 | 32.62 | 25.87 | 21.34 | 49.57 | 5.05 |
| | GH5 5 | 0.00 | 0.00 | 27.50 | 15.52 | 10.16 | 0.00 | 16.22 | 0.00 |
| | GH5 7 | 81.94 | 63.70 | 39.28 | 28.97 | 58.80 | 22.32 | 45.90 | 0.00 |
| | GH5 8 | 0.00 | 5.27 | 0.00 | 0.00 | 22.49 | 0.00 | 7.59 | 0.00 |
| | GH50 | 0.00 | 10.91 | 65.13 | 21.33 | 12.67 | 18.29 | 48.65 | 0.00 |
| | GH51 | 524.24 | 256.48 | 491.79 | 335.18 | 306.18 | 398.33 | 363.29 | 423.41 |
| | GH53 | 267.78 | 138.69 | 219.91 | 126.00 | 288.99 | 139.02 | 188.89 | 317.88 |
| | GH55 | 7.74 | 93.82 | 70.11 | 60.17 | 64.99 | 51.81 | 139.35 | 4.02 |
| | GH57 | 127.23 | 88.99 | 130.66 | 81.13 | 74.98 | 128.69 | 104.62 | 45.61 |
| | GH59 | 0.00 | 10.36 | 0.00 | 0.00 | 0.00 | 0.00 | 11.59 | 0.00 |
| | GH63 | 27.21 | 201.75 | 264.19 | 170.30 | 183.41 | 198.27 | 150.95 | 66.61 |
| | GH64 | 0.00 | 0.00 | 0.00 | 0.00 | 0.00 | 0.00 | 0.00 | 0.00 |
| | GH65 | 32.81 | 93.04 | 126.07 | 62.38 | 40.95 | 53.17 | 62.73 | 7.65 |
| | GH66 | 0.00 | 44.10 | 73.33 | 50.53 | 18.94 | 18.31 | 59.63 | 13.66 |
| | GH67 | 100.24 | 25.40 | 69.84 | 21.83 | 41.06 | 31.18 | 51.61 | 19.67 |
| | GH68 | 0.00 | 0.00 | 0.00 | 0.00 | 0.00 | 0.00 | 0.00 | 0.00 |
| | GH70 | 0.00 | 0.00 | 0.00 | 0.00 | 0.00 | 0.00 | 0.00 | 0.00 |
| | GH73 | 334.99 | 161.11 | 250.22 | 342.63 | 433.76 | 374.08 | 400.37 | 433.49 |
| | GH74 | 0.00 | 59.62 | 38.14 | 46.10 | 38.61 | 21.21 | 70.13 | 3.63 |
| | GH76 | 5.35 | 109.81 | 456.89 | 256.84 | 80.22 | 149.83 | 361.59 | 8.48 |
| | GH77 | 425.91 | 360.27 | 301.07 | 315.87 | 339.75 | 329.89 | 336.23 | 446.50 |
| | GH78 | 261.62 | 314.85 | 670.06 | 362.61 | 194.09 | 426.35 | 416.24 | 258.31 |
| | GH79 | 0.00 | 5.29 | 0.00 | 0.00 | 0.00 | 0.00 | 0.00 | 0.00 |
| | GH8 | 159.08 | 86.55 | 16.23 | 44.11 | 94.31 | 17.67 | 57.08 | 80.94 |
| | GH81 | 0.00 | 0.00 | 0.00 | 0.00 | 0.00 | 0.00 | 0.00 | 49.14 |
| | GH84 | 108.48 | 91.98 | 111.24 | 71.33 | 122.71 | 109.93 | 81.03 | 29.89 |
| | GH85 | 0.00 | 0.00 | 22.06 | 0.00 | 40.49 | 12.77 | 23.49 | 13.90 |
| | GH87 | 0.00 | 0.00 | 0.00 | 0.00 | 0.00 | 0.00 | 0.00 | 0.00 |
| | GH88 | 52.32 | 350.65 | 635.51 | 227.97 | 264.20 | 363.81 | 367.66 | 138.19 |
| | GH89 | 10.48 | 110.95 | 274.99 | 93.04 | 94.58 | 191.26 | 174.29 | 67.06 |
| | GH9 | 34.74 | 154.45 | 203.95 | 171.91 | 114.08 | 102.02 | 304.74 | 27.73 |
| | GH91 | 0.00 | 12.45 | 76.24 | 23.98 | 12.53 | 4.86 | 6.73 | 16.94 |
| | GH92 | 206.35 | 537.64 | 1461.67 | 859.90 | 573.04 | 1110.42 | 1156.65 | 321.35 |
| | GH93 | 0.00 | 0.00 | 0.00 | 0.00 | 0.00 | 0.00 | 0.00 | 0.00 |
| | GH94 | 310.79 | 197.40 | 155.17 | 194.94 | 300.70 | 114.16 | 208.92 | 358.50 |
| | GH95 | 327.13 | 295.50 | 473.67 | 262.57 | 299.86 | 431.68 | 371.47 | 181.68 |
| | GH97 | 443.18 | 763.76 | 1147.71 | 643.67 | 451.15 | 748.65 | 908.06 | 294.02 |
| | GH98 | 0.00 | 22.17 | 34.55 | 0.00 | 0.00 | 4.68 | 3.46 | 0.00 |
| | GH99 | 0.00 | 7.42 | 34.68 | 0.00 | 15.73 | 24.65 | 12.53 | 0.00 |
| | PL1 | 443.39 | 70.06 | 317.63 | 86.53 | 98.71 | 178.28 | 226.72 | 72.42 |
| | PL10 | 8.19 | 58.67 | 143.84 | 39.74 | 58.22 | 139.38 | 92.46 | 76.07 |
| | PL11 | 32.82 | 85.64 | 246.27 | 74.27 | 84.07 | 157.28 | 110.97 | 68.34 |
| | PL12 | 0.00 | 38.52 | 158.90 | 62.55 | 23.70 | 23.48 | 70.99 | 43.38 |
| | PL13 | 0.00 | 33.53 | 82.71 | 42.36 | 5.53 | 5.40 | 28.50 | 0.00 |
| | PL15 | 0.00 | 41.21 | 120.11 | 70.72 | 14.51 | 4.96 | 78.80 | 0.00 |
| | PL16 | 0.00 | 12.43 | 0.00 | 14.19 | 0.00 | 0.00 | 0.00 | 0.00 |
| | PL17 | 0.00 | 8.30 | 5.77 | 8.78 | 0.00 | 19.30 | 10.14 | 0.00 |
| | PL21 | 0.00 | 0.00 | 0.00 | 0.00 | 0.00 | 0.00 | 9.58 | 0.00 |
| | PL22 | 13.74 | 21.96 | 44.73 | 16.34 | 19.64 | 57.22 | 26.10 | 32.54 |
| | PL26 | 7.73 | 18.78 | 83.78 | 20.55 | 26.78 | 61.08 | 33.46 | 31.75 |
| | PL27 | 7.72 | 29.49 | 75.01 | 14.66 | 5.07 | 52.99 | 39.30 | 26.43 |
| | PL29 | 0.00 | 5.77 | 49.92 | 9.12 | 0.00 | 39.46 | 18.90 | 6.74 |
| | PL30 | 0.00 | 33.06 | 0.00 | 0.00 | 25.16 | 23.09 | 54.96 | 4.80 |
| | PL31 | 0.00 | 0.00 | 0.00 | 0.00 | 0.00 | 0.00 | 5.43 | 0.00 |
| | PL33 | 16.23 | 25.18 | 75.76 | 11.28 | 29.53 | 96.55 | 46.72 | 34.95 |
| | PL34 | 0.00 | 0.00 | 0.00 | 0.00 | 0.00 | 36.01 | 0.00 | 0.00 |
| | PL35 | 0.00 | 35.65 | 48.44 | 39.78 | 21.49 | 15.05 | 30.54 | 4.49 |
| | PL4 | 0.00 | 0.00 | 0.00 | 0.00 | 0.00 | 0.00 | 13.41 | 0.00 |
| | PL6 | 0.00 | 9.59 | 6.63 | 8.04 | 3.28 | 7.22 | 10.30 | 0.00 |
| | PL7 | 0.00 | 4.95 | 0.00 | 0.00 | 10.63 | 13.36 | 0.00 | 0.00 |
| | PL8 | 0.00 | 95.79 | 217.27 | 94.23 | 46.29 | 66.54 | 112.69 | 7.92 |
| | PL9 | 0.00 | 40.11 | 84.76 | 46.54 | 79.09 | 130.48 | 50.05 | 45.49 |
| | | Participant ID | | | | | | | |
| | | TP05.01 | TP05.02 | TP06.01 | TP06.02 | TP07.01 | TP07.02 | TP08.01 | TP08.02 |
| | | Timepoint | | | | | | | |
| | | Week 1 | Week 1 | Week 1 | Week 1 | Week 1 | Week 1 | Week 1 | Week 1 |
| | | Diet | | | | | | | |
| | | Free | Free | Free | Free | Free | Free | Free | Free |
| | | Diet | Diet | Diet | Diet | Diet | Diet | Diet | Diet |
| Read | GH1 | 356.03 | 270.88 | 163.03 | 302.70 | 179.01 | 412.19 | 243.18 | 221.74 |
| counts | GH10 | 168.99 | 37.30 | 63.44 | 10.96 | 159.35 | 161.04 | 146.39 | 178.31 |
| of | GH101 | 4.41 | 0.00 | 5.95 | 0.00 | 14.22 | 26.87 | 0.00 | 5.85 |
| CAZyme | GH102 | 7.49 | 0.00 | 0.00 | 0.00 | 0.00 | 0.00 | 7.17 | 16.59 |
| genes | GH103 | 0.00 | 0.00 | 0.00 | 0.00 | 0.00 | 0.00 | 0.00 | 0.00 |
| (TPM) | GH105 | 143.39 | 198.75 | 304.63 | 130.50 | 530.91 | 499.37 | 451.55 | 520.63 |
| normal- | GH106 | 125.94 | 32.39 | 74.63 | 26.27 | 195.22 | 119.86 | 162.73 | 139.57 |
| ized | GH108 | 68.25 | 47.76 | 30.89 | 76.09 | 55.50 | 9.48 | 5.09 | 98.43 |
| | GH109 | 35.62 | 62.40 | 178.37 | 136.55 | 143.35 | 185.92 | 181.74 | 149.40 |
| | GH110 | 28.41 | 46.14 | 153.88 | 82.18 | 99.20 | 103.44 | 86.22 | 65.71 |
| | GH111 | 0.00 | 0.00 | 0.00 | 0.00 | 9.00 | 0.00 | 0.00 | 0.00 |
| | GH112 | 60.34 | 129.77 | 60.29 | 85.44 | 74.01 | 147.59 | 118.33 | 84.11 |
| | GH113 | 6.26 | 4.91 | 14.72 | 0.00 | 14.22 | 22.57 | 19.37 | 4.47 |
| | GH114 | 16.10 | 11.98 | 0.00 | 0.00 | 0.00 | 15.10 | 0.00 | 14.89 |
| | GH115 | 150.37 | 17.41 | 54.94 | 23.77 | 147.46 | 149.35 | 321.93 | 197.79 |
| | GH116 | 12.68 | 0.00 | 10.42 | 5.32 | 50.63 | 33.29 | 53.17 | 41.59 |
| | GH117 | 0.00 | 0.00 | 22.42 | 24.49 | 28.12 | 44.34 | 13.16 | 50.07 |
| | GH120 | 37.30 | 45.82 | 39.18 | 47.12 | 61.34 | 76.59 | 106.80 | 32.09 |
| | GH121 | 0.00 | 0.00 | 13.88 | 0.00 | 6.28 | 0.00 | 0.00 | 0.00 |
| | GH123 | 28.90 | 30.20 | 89.08 | 80.61 | 99.96 | 83.87 | 64.46 | 80.26 |
| | GH125 | 58.72 | 26.77 | 99.55 | 150.12 | 142.85 | 165.54 | 132.12 | 159.97 |
| | GH126 | 0.00 | 0.00 | 0.00 | 0.00 | 0.00 | 0.00 | 0.00 | 0.00 |
| | GH127 | 228.03 | 72.33 | 138.30 | 88.49 | 159.78 | 141.06 | 162.85 | 212.10 |
| | GH128 | 0.00 | 16.77 | 13.32 | 0.00 | 0.00 | 0.00 | 0.00 | 8.07 |
| | GH129 | 14.24 | 29.86 | 14.04 | 8.65 | 8.25 | 6.78 | 3.08 | 4.11 |
| | GH13 | 727.73 | 529.19 | 400.75 | 469.15 | 583.92 | 542.51 | 497.78 | 539.35 |
| | GH13 11 | 77.14 | 119.26 | 75.58 | 103.98 | 63.95 | 122.43 | 79.68 | 121.24 |
| | GH13 12 | 8.29 | 0.00 | 0.00 | 0.00 | 0.00 | 0.00 | 0.00 | 7.16 |
| | GH13 14 | 122.58 | 218.71 | 203.76 | 211.92 | 149.70 | 227.04 | 212.38 | 180.78 |
| | GH13 18 | 38.25 | 29.02 | 28.01 | 41.33 | 16.51 | 33.40 | 24.65 | 60.52 |
| | GH13 19 | 34.54 | 40.56 | 32.92 | 47.35 | 23.34 | 31.04 | 12.28 | 17.27 |
| | GH13 2 | 0.00 | 6.23 | 0.00 | 0.00 | 0.00 | 0.00 | 0.00 | 0.00 |
| | GH13 20 | 48.26 | 63.05 | 10.15 | 28.06 | 55.32 | 81.57 | 99.48 | 80.86 |
| | GH13 28 | 40.27 | 100.84 | 80.72 | 124.95 | 48.71 | 66.67 | 65.24 | 41.34 |
| | GH13 29 | 17.91 | 33.46 | 21.43 | 0.00 | 4.35 | 31.29 | 9.51 | 19.45 |
| | GH13 3 | 0.00 | 0.00 | 17.39 | 17.86 | 0.00 | 0.00 | 0.00 | 0.00 |
| | GH13 30 | 0.00 | 4.58 | 67.16 | 40.77 | 0.00 | 0.00 | 0.00 | 0.00 |
| | GH13 31 | 134.37 | 155.98 | 140.13 | 191.44 | 200.14 | 133.96 | 138.58 | 183.89 |
| | GH13 32 | 0.00 | 3.67 | 47.58 | 13.19 | 0.00 | 0.00 | 0.00 | 0.00 |
| | GH13 36 | 65.55 | 68.65 | 37.42 | 42.36 | 55.93 | 50.32 | 51.22 | 68.83 |
| | GH13 37 | 0.00 | 0.00 | 0.00 | 0.00 | 0.00 | 0.00 | 0.00 | 0.00 |
| | GH13 38 | 60.63 | 31.44 | 62.23 | 44.30 | 58.31 | 50.94 | 87.18 | 68.52 |
| | GH13 39 | 79.07 | 179.58 | 164.28 | 217.71 | 110.97 | 103.74 | 81.06 | 81.35 |
| | GH13 4 | 76.14 | 69.02 | 27.88 | 38.08 | 54.91 | 58.90 | 74.14 | 80.28 |
| | GH13 41 | 7.00 | 18.39 | 4.73 | 4.39 | 0.00 | 12.73 | 28.83 | 44.55 |
| | GH13 42 | 0.00 | 0.00 | 0.00 | 0.00 | 0.00 | 0.00 | 24.80 | 0.00 |
| | GH13 5 | 32.47 | 11.24 | 10.05 | 17.08 | 60.56 | 7.51 | 25.96 | 18.34 |
| | GH13 8 | 80.46 | 27.90 | 76.80 | 60.01 | 71.96 | 47.69 | 89.34 | 64.30 |
| | GH13 9 | 321.33 | 382.37 | 255.43 | 359.94 | 268.19 | 297.90 | 264.18 | 247.20 |
| | GH130 | 237.77 | 69.07 | 183.67 | 139.28 | 242.78 | 349.87 | 258.66 | 168.13 |
| | GH133 | 110.28 | 50.28 | 56.67 | 55.28 | 107.75 | 103.04 | 123.01 | 87.66 |
| | GH136 | 14.25 | 18.25 | 46.74 | 28.56 | 81.22 | 44.83 | 61.27 | 58.89 |
| | GH137 | 15.29 | 0.00 | 10.71 | 0.00 | 17.22 | 24.81 | 54.23 | 25.44 |
| | GH138 | 14.81 | 8.28 | 11.34 | 6.74 | 25.37 | 36.04 | 116.17 | 17.88 |
| | GH139 | 0.00 | 15.98 | 11.40 | 0.00 | 42.22 | 48.20 | 63.36 | 32.21 |
| | GH140 | 26.34 | 34.47 | 23.62 | 12.96 | 64.40 | 74.18 | 85.84 | 59.09 |
| | GH141 | 76.46 | 7.95 | 45.92 | 18.10 | 55.06 | 109.04 | 91.78 | 76.92 |
| | GH142 | 5.34 | 8.20 | 10.88 | 0.00 | 32.82 | 47.04 | 80.17 | 39.17 |
| | GH143 | 0.00 | 8.20 | 10.88 | 0.00 | 26.52 | 34.61 | 72.65 | 32.29 |
| | GH144 | 3.88 | 17.59 | 54.04 | 84.90 | 187.69 | 189.54 | 131.02 | 172.47 |
| | GH145 | 17.08 | 0.00 | 20.46 | 34.70 | 53.10 | 48.78 | 33.31 | 65.90 |
| | GH146 | 20.05 | 26.76 | 111.44 | 57.10 | 161.90 | 140.32 | 135.81 | 187.88 |
| | GH147 | 0.00 | 0.00 | 12.88 | 0.00 | 27.50 | 10.10 | 27.83 | 7.94 |
| | GH148 | 2.75 | 0.00 | 8.60 | 6.31 | 0.00 | 4.27 | 3.71 | 0.00 |
| | GH15 | 12.40 | 54.19 | 19.29 | 15.88 | 12.64 | 0.00 | 28.49 | 18.07 |
| | GH151 | 15.17 | 8.04 | 8.94 | 3.65 | 22.45 | 10.07 | 5.30 | 9.75 |
| | GH153 | 4.93 | 0.00 | 0.00 | 0.00 | 0.00 | 0.00 | 0.00 | 0.00 |
| | GH154 | 72.79 | 56.26 | 46.02 | 34.20 | 157.84 | 183.23 | 78.85 | 142.97 |
| | GH155 | 0.00 | 2.17 | 42.64 | 0.00 | 69.85 | 114.29 | 48.75 | 104.06 |
| | GH156 | 0.00 | 0.00 | 4.33 | 0.00 | 6.76 | 0.00 | 8.11 | 0.00 |
| | GH157 | 0.00 | 0.00 | 0.00 | 0.00 | 17.14 | 10.80 | 0.00 | 0.00 |
| | GH158 | 0.00 | 0.00 | 5.56 | 27.13 | 17.21 | 31.41 | 0.00 | 30.34 |
| | GH159 | 0.00 | 0.00 | 0.00 | 0.00 | 44.94 | 39.56 | 9.10 | 0.00 |
| | GH16 | 146.83 | 114.22 | 155.70 | 80.76 | 243.12 | 196.48 | 200.85 | 180.78 |
| | GH161 | 5.1 | 5.75 | 0.00 | 0.00 | 0.00 | 0.00 | 0.00 | 7.67 |
| | GH17 | 0.00 | 0.00 | 0.00 | 0.00 | 0.00 | 0.00 | 0.00 | 0.00 |
| | GH18 | 123.27 | 181.51 | 226.14 | 189.03 | 433.26 | 583.76 | 286.03 | 491.66 |
| | GH19 | 19.57 | 0.00 | 0.00 | 0.00 | 0.00 | 0.00 | 0.00 | 8.54 |
| | GH2 | 1376.89 | 812.85 | 1215.22 | 1395.73 | 2109.07 | 2233.44 | 2085.27 | 2284.10 |
| | GH20 | 256.04 | 267.95 | 614.15 | 516.96 | 705.40 | 710.95 | 613.52 | 591.03 |
| | GH23 | 412.00 | 458.37 | 439.95 | 524.09 | 450.80 | 616.16 | 487.34 | 546.42 |
| | GH24 | 147.98 | 226.11 | 243.43 | 243.50 | 353.79 | 97.72 | 226.29 | 184.53 |
| | GH25 | 714.84 | 472.97 | 436.80 | 484.48 | 311.34 | 545.41 | 341.46 | 390.12 |
| | GH26 | 185.36 | 68.40 | 93.89 | 86.42 | 191.52 | 292.68 | 203.14 | 126.65 |
| | GH27 | 77.77 | 82.06 | 104.06 | 138.08 | 154.32 | 209.46 | 79.34 | 191.71 |
| | GH28 | 444.81 | 291.46 | 352.58 | 236.82 | 795.65 | 734.78 | 650.10 | 707.38 |
| | GH29 | 153.88 | 137.79 | 492.22 | 377.94 | 494.20 | 500.09 | 490.04 | 446.76 |
| | GH3 | 842.60 | 755.43 | 781.93 | 1242.39 | 1607.41 | 1668.59 | 1531.65 | 1604.24 |
| | GH30 | 0.00 | 16.40 | 0.00 | 0.00 | 4.43 | 0.00 | 10.10 | 0.00 |
| | GH30 1 | 5.63 | 13.05 | 0.00 | 16.12 | 0.00 | 8.21 | 4.07 | 9.72 |
| | GH30 2 | 2.88 | 6.17 | 32.44 | 17.00 | 65.94 | 71.88 | 0.00 | 46.76 |
| | GH30 3 | 21.73 | 3.01 | 18.70 | 27.37 | 69.55 | 63.84 | 23.75 | 48.95 |
| | GH30 4 | 48.37 | 18.83 | 167.43 | 144.66 | 103.19 | 99.32 | 47.90 | 106.71 |
| | GH30 5 | 0.00 | 5.84 | 4.56 | 0.00 | 0.00 | 0.00 | 0.00 | 0.00 |
| | GH30 6 | 0.00 | 3.10 | 18.47 | 0.00 | 5.30 | 0.00 | 14.07 | 0.00 |
| | GH30 8 | 5.64 | 0.00 | 0.00 | 0.00 | 24.37 | 15.91 | 0.00 | 8.10 |
| | GH31 | 355.61 | 331.46 | 306.58 | 509.59 | 582.72 | 720.11 | 554.29 | 654.85 |
| | GH32 | 365.36 | 260.01 | 196.73 | 350.32 | 367.69 | 418.63 | 343.04 | 454.80 |
| | GH33 | 112.08 | 79.60 | 325.76 | 214.78 | 213.03 | 257.47 | 211.50 | 204.22 |
| | GH35 | 87.83 | 6.03 | 134.19 | 115.69 | 130.51 | 173.04 | 144.72 | 149.05 |
| | GH36 | 405.40 | 205.73 | 291.18 | 299.21 | 392.72 | 483.55 | 354.33 | 309.45 |
| | GH37 | 3.02 | 0.00 | 0.00 | 0.00 | 0.00 | 0.00 | 0.00 | 0.00 |
| | GH38 | 38.35 | 68.65 | 173.02 | 220.88 | 114.81 | 160.07 | 109.17 | 122.50 |
| | GH39 | 27.96 | 22.30 | 53.86 | 18.93 | 46.85 | 85.05 | 47.45 | 43.43 |
| | GH4 | 45.00 | 15.31 | 90.06 | 79.22 | 10.39 | 121.78 | 106.68 | 47.58 |
| | GH42 | 106.53 | 102.14 | 144.50 | 139.34 | 183.48 | 261.03 | 141.10 | 191.72 |
| | GH43 | 0.00 | 5.26 | 87.73 | 7.63 | 40.99 | 58.25 | 18.52 | 13.25 |
| | GH43 1 | 79.77 | 10.52 | 19.69 | 0.00 | 26.47 | 38.17 | 61.47 | 35.57 |
| | GH43 10 | 236.51 | 110.30 | 160.91 | 107.74 | 206.62 | 229.93 | 316.47 | 289.37 |
| | GH43 11 | 35.66 | 32.45 | 79.97 | 33.25 | 15.55 | 28.63 | 30.07 | 17.62 |
| | GH43 12 | 80.06 | 48.20 | 102.10 | 61.26 | 131.93 | 160.50 | 222.74 | 158.88 |
| | GH43 16 | 0.00 | 28.81 | 20.83 | 23.65 | 85.60 | 47.16 | 66.27 | 45.61 |
| | GH43 17 | 7.59 | 14.10 | 76.40 | 29.67 | 50.25 | 23.12 | 9.57 | 50.24 |
| | GH43 18 | 10.71 | 17.46 | 31.77 | 0.00 | 41.23 | 94.15 | 81.24 | 61.68 |
| | GH43 19 | 80.71 | 51.32 | 18.46 | 30.24 | 98.56 | 73.16 | 51.42 | 102.72 |
| | GH43 2 | 67.10 | 0.00 | 13.65 | 0.00 | 70.13 | 35.50 | 126.67 | 27.93 |
| | GH43 22 | 31.84 | 9.54 | 67.51 | 62.48 | 17.22 | 0.00 | 16.12 | 0.00 |
| | GH43 24 | 80.48 | 39.19 | 58.22 | 70.59 | 181.64 | 89.71 | 101.16 | 224.59 |
| | GH43 26 | 46.41 | 39.65 | 51.87 | 32.88 | 56.64 | 101.27 | 52.95 | 55.58 |
| | GH43 27 | 0.00 | 0.00 | 17.04 | 15.38 | 0.00 | 3.62 | 11.41 | 0.00 |
| | GH43 28 | 0.00 | 12.59 | 4.70 | 58.30 | 133.23 | 143.50 | 124.07 | 95.51 |
| | GH43 29 | 158.78 | 6.84 | 89.63 | 0.00 | 81.64 | 102.65 | 295.02 | 73.57 |
| | GH43 3 | 0.00 | 16.12 | 14.86 | 0.00 | 95.92 | 112.89 | 54.98 | 73.99 |
| | GH43 30 | 0.00 | 0.00 | 0.00 | 0.00 | 0.00 | 0.00 | 6.18 | 0.00 |
| | GH43 31 | 15.95 | 16.34 | 14.35 | 22.72 | 101.74 | 90.76 | 64.91 | 143.72 |
| | GH43 32 | 6.00 | 22.99 | 14.42 | 31.44 | 0.00 | 0.00 | 0.00 | 0.00 |
| | GH43 33 | 0.00 | 0.00 | 0.00 | 37.86 | 31.54 | 9.80 | 27.25 | 29.54 |
| | GH43 34 | 68.35 | 30.64 | 78.46 | 35.69 | 156.05 | 179.70 | 84.38 | 153.92 |
| | GH43 35 | 66.78 | 73.06 | 31.61 | 55.39 | 50.69 | 69.54 | 127.74 | 76.19 |
| | GH43 37 | 0.00 | 0.00 | 0.00 | 0.00 | 0.00 | 0.00 | 0.00 | 0.00 |
| | GH43 4 | 88.74 | 46.84 | 43.79 | 8.66 | 65.59 | 38.10 | 125.36 | 96.28 |
| | GH43 5 | 70.69 | 0.00 | 0.00 | 0.00 | 0.00 | 0.00 | 0.00 | 0.00 |
| | GH43 7 | 62.08 | 0.00 | 18.12 | 0.00 | 16.37 | 11.10 | 53.43 | 9.65 |
| | GH43 8 | 0.00 | 0.00 | 0.00 | 0.00 | 24.48 | 0.00 | 11.72 | 24.22 |
| | GH43 9 | 12.35 | 8.22 | 10.36 | 0.00 | 31.37 | 10.48 | 17.90 | 50.16 |
| | GH44 | 14.67 | 0.00 | 0.00 | 0.00 | 0.00 | 7.38 | 0.00 | 0.00 |
| | GH48 | 0.00 | 0.00 | 0.00 | 0.00 | 0.00 | 10.48 | 0.00 | 0.00 |
| | GH5 | 55.41 | 22.80 | 111.20 | 71.29 | 44.23 | 41.12 | 22.94 | 27.31 |
| | GH5 1 | 17.58 | 7.34 | 13.15 | 0.00 | 0.00 | 6.75 | 13.44 | 5.79 |
| | GH5 13 | 0.00 | 0.00 | 10.54 | 0.00 | 4.08 | 54.32 | 25.28 | 6.19 |
| | GH5 18 | 0.00 | 0.00 | 0.00 | 0.00 | 0.00 | 0.00 | 0.00 | 0.00 |
| | GH5 2 | 146.14 | 65.71 | 27.18 | 29.64 | 80.71 | 90.53 | 74.39 | 99.61 |
| | GH5 21 | 68.45 | 0.00 | 16.91 | 0.00 | 36.42 | 48.98 | 62.01 | 17.57 |
| | GH5 22 | 0.00 | 0.00 | 0.00 | 0.00 | 0.00 | 0.00 | 0.00 | 0.00 |
| | GH5 25 | 0.00 | 0.00 | 0.00 | 0.00 | 0.00 | 0.00 | 0.00 | 0.00 |
| | GH5 26 | 0.00 | 0.00 | 0.00 | 0.00 | 0.00 | 0.00 | 0.00 | 0.00 |
| | GH5 27 | 0.00 | 0.00 | 9.73 | 23.98 | 0.00 | 0.00 | 0.00 | 0.00 |
| | GH5 37 | 33.74 | 37.62 | 10.18 | 0.00 | 14.34 | 19.79 | 17.92 | 5.59 |
| | GH5 38 | 0.62 | 0.00 | 0.00 | 0.00 | 6.93 | 0.00 | 7.99 | 0.00 |
| | GH5 4 | 178.02 | 84.82 | 88.43 | 59.45 | 71.67 | 200.82 | 117.73 | 114.48 |
| | GH5 44 | 0.00 | 7.32 | 39.91 | 38.72 | 0.00 | 0.00 | 0.00 | 2.43 |
| | GH5 46 | 0.00 | 0.00 | 12.84 | 27.89 | 39.05 | 22.34 | 15.11 | 47.96 |
| | GH5 5 | 0.00 | 0.00 | 0.00 | 0.00 | 3.72 | 52.73 | 18.62 | 5.49 |
| | GH5 7 | 73.98 | 11.40 | 20.65 | 29.66 | 50.65 | 72.25 | 77.72 | 46.01 |
| | GH5 8 | 7.51 | 12.73 | 27.57 | 0.00 | 0.00 | 17.20 | 15.33 | 0.00 |
| | GH50 | 0.00 | 2.87 | 22.28 | 27.38 | 40.86 | 135.86 | 24.16 | 31.93 |
| | GH51 | 400.74 | 138.99 | 179.87 | 180.60 | 409.55 | 344.01 | 378.29 | 351.57 |
| | GH53 | 177.98 | 48.13 | 27.69 | 29.52 | 95.41 | 96.02 | 189.36 | 189.72 |
| | GH55 | 5.61 | 13.42 | 16.91 | 115.98 | 40.73 | 19.75 | 48.81 | 98.01 |
| | GH57 | 95.83 | 62.75 | 71.86 | 61.97 | 99.38 | 65.19 | 111.59 | 97.37 |
| | GH59 | 8.68 | 10.47 | 11.18 | 0.00 | 5.18 | 7.86 | 25.86 | 0.00 |
| | GH63 | 67.18 | 44.99 | 121.88 | 111.45 | 139.73 | 105.41 | 194.66 | 219.58 |
| | GH64 | 0.00 | 0.00 | 0.00 | 0.00 | 0.00 | 0.00 | 0.00 | 0.00 |
| | GH65 | 5.53 | 19.98 | 33.25 | 36.70 | 88.78 | 107.28 | 149.21 | 72.61 |
| | GH66 | 18.67 | 0.00 | 16.80 | 26.76 | 34.85 | 55.59 | 30.48 | 51.94 |
| | GH67 | 71.44 | 6.84 | 36.48 | 7.54 | 47.19 | 51.67 | 123.03 | 46.05 |
| | GH68 | 12.93 | 0.00 | 0.00 | 0.00 | 0.00 | 0.00 | 0.00 | 8.16 |
| | GH70 | 48.62 | 0.00 | 0.00 | 0.00 | 0.00 | 0.00 | 0.00 | 21.38 |
| | GH73 | 519.86 | 400.21 | 304.17 | 303.83 | 345.24 | 346.15 | 373.48 | 383.23 |
| | GH74 | 22.22 | 11.51 | 118.16 | 28.78 | 18.71 | 30.86 | 35.53 | 33.82 |
| | GH76 | 3.20 | 16.80 | 67.57 | 77.23 | 107.21 | 213.25 | 170.28 | 171.28 |
| | GH77 | 335.58 | 429.78 | 286.59 | 409.79 | 311.38 | 391.45 | 353.55 | 300.46 |
| | GH78 | 142.04 | 139.85 | 203.58 | 168.86 | 392.08 | 447.74 | 393.52 | 345.93 |
| | GH79 | 3.26 | 4.49 | 0.00 | 0.00 | 14.49 | 8.42 | 0.00 | 8.13 |
| | GH8 | 104.96 | 65.90 | 47.05 | 39.40 | 46.78 | 41.79 | 194.50 | 88.36 |
| | GH81 | 0.00 | 38.34 | 8.72 | 0.00 | 0.00 | 0.00 | 0.00 | 0.00 |
| | GH84 | 9.41 | 4.32 | 47.60 | 62.45 | 44.66 | 54.06 | 68.74 | 66.07 |
| | GH85 | 0.00 | 9.86 | 0.00 | 30.44 | 4.40 | 7.22 | 7.65 | 2.81 |
| | GH87 | 11.14 | 0.00 | 0.00 | 0.00 | 0.00 | 0.00 | 0.00 | 6.56 |
| | GH88 | 23.63 | 54.42 | 156.12 | 165.96 | 291.23 | 352.91 | 446.19 | 382.66 |
| | GH89 | 26.93 | 40.18 | 120.00 | 59.89 | 113.74 | 92.13 | 104.57 | 108.22 |
| | GH9 | 57.83 | 59.64 | 35.96 | 108.39 | 149.53 | 122.40 | 66.64 | 136.37 |
| | GH91 | 0.00 | 0.00 | 30.04 | 0.00 | 6.33 | 4.96 | 4.62 | 0.00 |
| | GH92 | 146.51 | 189.88 | 411.76 | 260.83 | 803.72 | 900.80 | 756.51 | 702.70 |
| | GH93 | 0.00 | 5.25 | 0.00 | 0.00 | 0.00 | 0.00 | 0.00 | 0.00 |
| | GH94 | 259.32 | 254.40 | 156.58 | 145.44 | 201.52 | 159.33 | 277.39 | 205.85 |
| | GH95 | 301.47 | 129.17 | 262.89 | 169.82 | 451.65 | 438.29 | 388.89 | 341.01 |
| | GH97 | 386.44 | 124.22 | 370.87 | 411.76 | 636.03 | 581.77 | 742.15 | 820.32 |
| | GH98 | 13.90 | 0.00 | 9.61 | 0.00 | 11.77 | 13.17 | 0.00 | 0.00 |
| | GH99 | 0.00 | 4.38 | 0.00 | 0.00 | 6.70 | 59.22 | 15.25 | 14.47 |
| | PL1 | 108.48 | 11.36 | 140.11 | 8.50 | 142.16 | 197.36 | 212.47 | 106.37 |
| | PL10 | 0.00 | 22.19 | 44.62 | 0.00 | 60.40 | 45.52 | 76.85 | 64.57 |
| | PL11 | 24.77 | 27.84 | 47.39 | 15.64 | 71.10 | 141.65 | 53.92 | 93.12 |
| | PL12 | 20.66 | 0.00 | 63.34 | 28.93 | 39.50 | 56.05 | 137.42 | 71.74 |
| | PL13 | 0.00 | 0.00 | 15.65 | 26.99 | 42.17 | 34.19 | 74.64 | 47.00 |
| | PL15 | 0.00 | 0.00 | 22.11 | 27.89 | 33.84 | 92.21 | 298.48 | 51.18 |
| | PL16 | 6.99 | 8.70 | 18.24 | 0.00 | 10.91 | 0.00 | 0.00 | 19.71 |
| | PL17 | 0.00 | 7.37 | 0.00 | 0.00 | 0.00 | 41.90 | 106.01 | 15.50 |
| | PL21 | 0.00 | 0.00 | 29.15 | 0.00 | 0.00 | 0.00 | 98.70 | 0.00 |
| | PL22 | 5.15 | 7.79 | 12.37 | 5.21 | 10.37 | 0.00 | 12.95 | 20.28 |
| | PL26 | 4.76 | 7.17 | 10.41 | 5.87 | 17.20 | 25.93 | 18.50 | 22.76 |
| | PL27 | 6.88 | 9.73 | 12.36 | 7.21 | 17.10 | 20.16 | 25.10 | 4.26 |
| | PL29 | 0.00 | 0.00 | 7.65 | 0.00 | 6.87 | 25.30 | 21.07 | 10.67 |
| | PL30 | 3.53 | 0.00 | 14.81 | 24.76 | 17.95 | 22.31 | 23.43 | 33.45 |
| | PL31 | 0.00 | 4.53 | 0.00 | 0.00 | 0.00 | 6.96 | 0.00 | 0.00 |
| | PL33 | 16.79 | 23.64 | 27.39 | 9.57 | 56.13 | 78.20 | 89.48 | 25.90 |
| | PL34 | 0.00 | 15.77 | 0.00 | 0.00 | 0.00 | 0.00 | 11.56 | 0.00 |
| | PL35 | 3.12 | 3.99 | 30.04 | 13.16 | 23.87 | 59.25 | 98.53 | 38.92 |
| | PL4 | 0.00 | 0.00 | 0.00 | 0.00 | 0.00 | 0.00 | 0.00 | 0.00 |
| | PL6 | 0.00 | 0.00 | 0.00 | 0.00 | 14.05 | 42.11 | 49.38 | 18.20 |
| | PL7 | 0.00 | 0.00 | 0.00 | 0.00 | 0.00 | 0.00 | 10.28 | 0.00 |
| | PL8 | 38.73 | 0.00 | 81.12 | 27.14 | 171.71 | 149.78 | 144.82 | 117.89 |
| | PL9 | 10.85 | 37.94 | 20.41 | 0.00 | 37.23 | 57.29 | 9.13 | 31.68 |

| | | Participant ID | | | | | |
|---|---|---|---|---|---|---|---|
| | | TP09.01 | TP09.02 | TP10.02 | TP11.01 | TP12.01 | TP12.02 |
| | | Timepoint | | | | | |
| | | Week 1 | Week 1 | Week 1 | Week 1 | Week 1 | Week 1 |
| | | Diet | | | | | |
| | | Free | Free | Free | Free | Free | Free |
| | | Diet | Diet | Diet | Diet | Diet | Diet |
| Read | GH1 | 173.51 | 282.26 | 373.88 | 120.71 | 459.68 | 135.89 |
| counts | GH10 | 97.46 | 92.91 | 119.69 | 152.64 | 40.73 | 136.48 |
| of | GH101 | 32.68 | 0.00 | 14.40 | 0.00 | 21.51 | 41.83 |
| CAZyme | GH102 | 18.19 | 8.77 | 0.00 | 0.00 | 6.04 | 0.00 |
| genes | GH103 | 7.36 | 0.00 | 0.00 | 0.00 | 0.00 | 3.09 |
| (TPM) | GH105 | 438.80 | 199.04 | 393.66 | 270.36 | 229.81 | 871.97 |
| normal- | GH106 | 135.20 | 168.67 | 107.34 | 171.56 | 47.94 | 258.47 |
| ized | GH108 | 52.37 | 58.99 | 210.32 | 40.07 | 17.40 | 530.44 |
| | GH109 | 265.31 | 127.64 | 123.71 | 72.86 | 40.99 | 432.47 |
| | GH110 | 166.04 | 139.44 | 64.92 | 24.19 | 79.83 | 158.77 |
| | GH111 | 0.00 | 0.00 | 0.00 | 0.00 | 0.00 | 3.69 |
| | GH112 | 64.43 | 84.85 | 147.06 | 71.42 | 133.23 | 69.48 |
| | GH113 | 0.00 | 8.90 | 20.65 | 20.01 | 13.47 | 11.54 |
| | GH114 | 8.57 | 0.00 | 12.45 | 0.00 | 13.22 | 14.08 |
| | GH115 | 53.14 | 106.61 | 162.93 | 322.95 | 70.28 | 245.91 |
| | GH116 | 55.08 | 6.45 | 125.95 | 5.13 | 4.32 | 56.83 |
| | GH117 | 26.19 | 0.00 | 33.35 | 0.00 | 0.00 | 26.59 |
| | GH120 | 44.90 | 17.91 | 123.23 | 22.68 | 16.50 | 47.52 |
| | GH121 | 0.00 | 0.00 | 0.00 | 0.00 | 0.00 | 0.00 |
| | GH123 | 170.51 | 92.78 | 52.54 | 24.58 | 32.80 | 161.73 |
| | GH125 | 139.68 | 48.14 | 153.01 | 31.28 | 15.33 | 251.24 |
| | GH126 | 7.06 | 0.00 | 0.00 | 0.00 | 0.00 | 0.00 |
| | GH127 | 160.58 | 232.70 | 253.30 | 496.40 | 55.59 | 231.20 |
| | GH128 | 14.26 | 0.00 | 0.00 | 0.00 | 0.00 | 0.00 |
| | GH129 | 10.03 | 15.65 | 0.00 | 0.00 | 6.78 | 6.17 |
| | GH13 | 466.92 | 418.66 | 532.73 | 921.35 | 474.85 | 526.55 |
| | GH13 11 | 46.33 | 74.54 | 248.67 | 123.58 | 111.15 | 129.40 |
| | GH13 12 | 0.00 | 0.00 | 0.00 | 0.00 | 0.00 | 0.00 |
| | GH13 14 | 126.93 | 143.76 | 142.16 | 170.08 | 307.82 | 182.09 |
| | GH13 18 | 0.00 | 17.06 | 114.38 | 18.18 | 44.10 | 38.37 |
| | GH13 19 | 22.07 | 0.00 | 0.00 | 0.00 | 39.35 | 12.91 |
| | GH13 2 | 0.00 | 0.00 | 0.00 | 0.00 | 0.00 | 0.00 |
| | GH13 20 | 13.87 | 43.00 | 180.18 | 60.27 | 62.73 | 48.31 |
| | GH13 28 | 23.85 | 17.40 | 61.13 | 0.00 | 88.80 | 22.93 |
| | GH13 29 | 20.34 | 10.33 | 19.70 | 0.00 | 33.85 | 0.00 |
| | GH13 3 | 0.00 | 0.00 | 0.00 | 0.00 | 8.32 | 0.00 |
| | GH13 30 | 0.00 | 7.56 | 0.00 | 0.00 | 0.00 | 0.00 |
| | GH13 31 | 103.95 | 158.87 | 364.26 | 204.95 | 131.33 | 201.78 |
| | GH13 32 | 0.00 | 0.00 | 0.00 | 0.00 | 0.00 | 0.00 |
| | GH13 36 | 16.89 | 32.54 | 120.67 | 20.05 | 73.35 | 54.03 |
| | GH13 37 | 0.00 | 0.00 | 0.00 | 0.00 | 0.00 | 0.00 |
| | GH13 38 | 92.33 | 70.77 | 53.73 | 138.85 | 33.71 | 95.08 |
| | GH13 39 | 53.11 | 138.64 | 55.90 | 25.89 | 160.97 | 36.46 |
| | GH13 4 | 33.21 | 50.40 | 49.06 | 53.87 | 87.15 | 31.25 |
| | GH13 41 | 0.00 | 21.77 | 14.02 | 16.67 | 35.09 | 29.28 |
| | GH13 42 | 0.00 | 0.00 | 0.00 | 0.00 | 0.00 | 0.00 |
| | GH13 5 | 50.48 | 26.57 | 21.33 | 0.00 | 13.19 | 0.00 |
| | GH13 8 | 118.74 | 74.23 | 53.09 | 115.97 | 59.55 | 108.65 |
| | GH13 9 | 164.67 | 250.23 | 311.80 | 206.57 | 389.80 | 167.65 |
| | GH130 | 193.56 | 159.67 | 262.71 | 178.00 | 163.73 | 454.27 |
| | GH133 | 121.98 | 62.06 | 127.34 | 131.70 | 65.14 | 139.53 |
| | GH136 | 60.13 | 47.13 | 102.68 | 32.20 | 46.82 | 124.99 |
| | GH137 | 18.40 | 49.14 | 25.69 | 11.56 | 0.00 | 74.26 |
| | GH138 | 21.61 | 46.30 | 35.39 | 12.98 | 0.00 | 17.31 |
| | GH139 | 17.83 | 16.24 | 21.55 | 16.51 | 0.00 | 71.61 |
| | GH140 | 50.93 | 67.72 | 48.83 | 23.25 | 27.09 | 151.04 |
| | GH141 | 59.92 | 89.80 | 52.73 | 151.97 | 22.52 | 187.06 |
| | GH142 | 40.71 | 10.02 | 23.91 | 12.04 | 11.79 | 59.68 |
| | GH143 | 18.69 | 0.00 | 23.91 | 12.04 | 0.00 | 49.10 |
| | GH144 | 125.03 | 23.88 | 80.16 | 0.00 | 0.00 | 148.74 |
| | GH145 | 52.74 | 14.66 | 59.18 | 13.15 | 17.84 | 133.51 |
| | GH146 | 118.26 | 51.38 | 111.01 | 148.65 | 13.51 | 226.88 |
| | GH147 | 7.25 | 0.00 | 0.00 | 0.00 | 0.00 | 49.70 |
| | GH148 | 0.00 | 0.00 | 11.81 | 0.00 | 0.00 | 8.55 |
| | GH15 | 34.51 | 22.18 | 43.99 | 21.80 | 8.08 | 95.83 |
| | GH151 | 9.54 | 0.00 | 0.00 | 7.52 | 0.00 | 12.25 |
| | GH153 | 0.00 | 0.00 | 0.00 | 0.00 | 0.00 | 0.00 |
| | GH154 | 116.28 | 40.75 | 91.13 | 199.78 | 74.89 | 246.16 |
| | GH155 | 31.60 | 3.51 | 8.24 | 0.00 | 5.79 | 234.25 |
| | GH156 | 12.42 | 0.00 | 0.00 | 0.00 | 0.00 | 0.00 |
| | GH157 | 0.00 | 0.00 | 0.00 | 0.00 | 0.00 | 0.00 |
| | GH158 | 0.00 | 0.00 | 16.43 | 0.00 | 0.00 | 24.69 |
| | GH159 | 24.78 | 0.00 | 0.00 | 0.00 | 0.00 | 0.00 |
| | GH16 | 359.73 | 179.27 | 133.62 | 279.96 | 143.22 | 263.58 |
| | GH161 | 0.00 | 6.46 | 0.00 | 0.00 | 0.00 | 7.99 |
| | GH17 | 0.00 | 0.00 | 0.00 | 0.00 | 8.30 | 0.00 |
| | GH18 | 416.65 | 164.24 | 365.23 | 209.30 | 145.48 | 576.27 |
| | GH19 | 9.25 | 0.00 | 0.00 | 0.00 | 0.00 | 0.00 |
| | GH2 | 1927.67 | 1540.18 | 1615.86 | 1817.94 | 1171.51 | 2982.93 |
| | GH20 | 1218.18 | 672.71 | 500.45 | 334.93 | 406.70 | 1112.99 |
| | GH23 | 605.33 | 528.34 | 798.91 | 620.47 | 563.91 | 696.48 |
| | GH24 | 292.77 | 142.14 | 172.25 | 63.39 | 178.82 | 271.82 |
| | GH25 | 282.32 | 550.32 | 530.99 | 656.42 | 503.07 | 253.12 |
| | GH26 | 170.57 | 118.66 | 109.00 | 121.60 | 85.19 | 121.09 |
| | GH27 | 187.83 | 138.34 | 94.64 | 32.66 | 62.79 | 273.52 |
| | GH28 | 445.82 | 445.75 | 493.12 | 825.45 | 270.66 | 1194.73 |
| | GH29 | 788.39 | 416.75 | 410.89 | 356.25 | 165.25 | 866.25 |
| | GH3 | 994.32 | 821.17 | 1603.26 | 1068.93 | 728.23 | 1630.33 |
| | GH30 | 0.00 | 0.00 | 0.00 | 0.00 | 0.00 | 19.66 |
| | GH30 1 | 2.47 | 0.00 | 0.00 | 15.31 | 0.00 | 29.40 |
| | GH30 2 | 0.00 | 8.66 | 0.00 | 12.26 | 0.00 | 46.82 |
| | GH30 3 | 45.57 | 21.27 | 31.75 | 117.94 | 12.30 | 51.75 |
| | GH30 4 | 96.97 | 90.53 | 105.19 | 171.67 | 14.44 | 238.84 |
| | GH30 5 | 0.00 | 0.00 | 0.00 | 0.00 | 0.00 | 7.16 |
| | GH30 6 | 30.07 | 12.52 | 31.45 | 13.89 | 10.04 | 73.55 |
| | GH30 8 | 0.00 | 0.00 | 0.00 | 0.00 | 0.00 | 23.57 |
| | GH31 | 508.93 | 418.13 | 674.93 | 489.59 | 340.69 | 824.04 |
| | GH32 | 207.96 | 306.16 | 533.80 | 389.39 | 300.27 | 388.12 |
| | GH33 | 396.58 | 332.88 | 143.91 | 169.51 | 166.30 | 412.61 |
| | GH35 | 207.66 | 176.95 | 131.93 | 278.30 | 19.16 | 274.65 |
| | GH36 | 398.59 | 413.18 | 411.23 | 420.88 | 276.81 | 445.13 |
| | GH37 | 6.63 | 0.00 | 0.00 | 0.00 | 0.00 | 0.00 |
| | GH38 | 120.20 | 99.28 | 112.29 | 11.04 | 48.60 | 235.82 |
| | GH39 | 22.80 | 45.09 | 12.14 | 19.73 | 66.91 | 36.70 |
| | GH4 | 56.53 | 95.64 | 127.90 | 150.39 | 23.32 | 34.50 |
| | GH42 | 86.28 | 87.18 | 274.54 | 78.38 | 111.26 | 218.63 |
| | GH43 | 18.79 | 0.00 | 0.00 | 0.00 | 9.81 | 76.04 |
| | GH43 1 | 12.12 | 44.40 | 24.61 | 148.16 | 0.00 | 74.86 |
| | GH43 10 | 126.71 | 222.95 | 103.42 | 552.96 | 99.63 | 379.52 |
| | GH43 11 | 0.00 | 0.00 | 58.02 | 6.51 | 24.78 | 36.62 |
| | GH43 12 | 97.10 | 95.04 | 218.23 | 183.91 | 89.76 | 274.65 |
| | GH43 16 | 34.69 | 3.90 | 54.36 | 0.00 | 5.25 | 40.04 |
| | GH43 17 | 33.17 | 33.10 | 27.44 | 20.56 | 10.54 | 98.72 |
| | GH43 18 | 43.08 | 9.63 | 24.14 | 22.79 | 16.39 | 200.56 |
| | GH43 19 | 84.34 | 72.49 | 75.42 | 164.75 | 23.67 | 178.23 |
| | GH43 2 | 14.71 | 43.98 | 19.15 | 130.95 | 0.00 | 44.90 |
| | GH43 22 | 1.63 | 0.00 | 9.79 | 0.00 | 0.00 | 18.94 |
| | GH43 24 | 123.79 | 50.16 | 120.31 | 167.25 | 30.39 | 295.75 |
| | GH43 26 | 50.97 | 34.95 | 107.98 | 21.49 | 12.38 | 70.61 |
| | GH43 27 | 0.00 | 0.00 | 0.00 | 0.00 | 0.00 | 15.93 |
| | GH43 28 | 81.81 | 40.14 | 38.86 | 3.07 | 50.24 | 77.80 |
| | GH43 29 | 62.42 | 95.38 | 80.71 | 306.72 | 32.53 | 108.16 |
| | GH43 3 | 55.02 | 0.00 | 41.62 | 10.28 | 9.57 | 139.30 |
| | GH43 30 | 8.96 | 0.00 | 0.00 | 0.00 | 0.00 | 0.00 |
| | GH43 31 | 85.57 | 4.13 | 115.26 | 19.92 | 13.16 | 231.87 |
| | GH43 32 | 0.00 | 0.00 | 0.00 | 4.19 | 0.00 | 0.00 |
| | GH43 33 | 50.61 | 0.00 | 28.37 | 0.00 | 13.46 | 34.89 |
| | GH43 34 | 160.08 | 75.98 | 104.93 | 176.48 | 42.65 | 336.36 |
| | GH43 35 | 28.98 | 56.74 | 101.60 | 2.67 | 64.79 | 78.00 |
| | GH43 37 | 0.00 | 0.00 | 0.00 | 0.00 | 0.00 | 0.00 |
| | GH43 4 | 100.92 | 89.64 | 160.78 | 334.54 | 26.58 | 174.03 |
| | GH43 5 | 0.00 | 0.00 | 0.00 | 143.63 | 8.97 | 0.00 |
| | GH43 7 | 11.40 | 35.95 | 0.00 | 128.37 | 0.00 | 0.00 |
| | GH43 8 | 10.12 | 0.00 | 0.00 | 0.00 | 0.00 | 13.70 |
| | GH43 9 | 25.27 | 11.16 | 42.34 | 22.63 | 7.62 | 115.18 |
| | GH44 | 0.00 | 9.01 | 10.25 | 0.00 | 0.00 | 0.00 |
| | GH48 | 22.36 | 0.00 | 14.63 | 0.00 | 10.20 | 0.00 |
| | GH5 | 39.51 | 104.84 | 22.30 | 0.00 | 46.77 | 0.00 |
| | GH5 1 | 43.44 | 16.49 | 9.88 | 0.00 | 37.63 | 0.00 |
| | GH5 13 | 3.15 | 0.00 | 0.00 | 0.00 | 4.30 | 57.77 |
| | GH5 18 | 0.00 | 0.00 | 0.00 | 0.00 | 0.00 | 0.00 |
| | GH5 2 | 27.78 | 37.28 | 97.41 | 12.80 | 83.28 | 66.54 |
| | GH5 21 | 12.46 | 42.46 | 0.00 | 249.20 | 0.00 | 0.00 |
| | GH5 22 | 0.00 | 0.00 | 0.00 | 0.00 | 0.00 | 0.00 |
| | GH5 25 | 0.00 | 0.00 | 0.00 | 0.00 | 0.00 | 0.00 |
| | GH5 26 | 0.00 | 0.00 | 0.00 | 0.00 | 0.00 | 0.00 |
| | GH5 27 | 0.00 | 0.00 | 0.00 | 4.51 | 0.00 | 0.00 |
| | GH5 37 | 10.86 | 11.46 | 8.53 | 0.00 | 18.79 | 0.00 |
| | GH5 38 | 9.16 | 0.00 | 0.00 | 0.00 | 0.00 | 0.00 |
| | GH5 4 | 226.36 | 170.61 | 133.13 | 425.84 | 69.52 | 98.13 |
| | GH5 44 | 0.00 | 0.00 | 0.00 | 0.00 | 0.00 | 0.00 |
| | GH5 46 | 67.77 | 0.00 | 33.59 | 0.00 | 5.90 | 27.93 |
| | GH5 5 | 20.83 | 0.00 | 0.00 | 0.00 | 6.50 | 47.48 |
| | GH5 7 | 36.60 | 47.55 | 39.08 | 121.84 | 15.82 | 26.90 |
| | GH5 8 | 6.22 | 11.14 | 0.00 | 0.00 | 17.57 | 0.00 |
| | GH50 | 6.84 | 0.00 | 0.00 | 0.00 | 0.00 | 59.67 |
| | GH51 | 272.08 | 300.61 | 469.94 | 582.79 | 148.22 | 493.62 |
| | GH53 | 82.19 | 94.12 | 345.07 | 214.15 | 164.66 | 230.19 |
| | GH55 | 41.18 | 18.73 | 76.63 | 0.00 | 6.33 | 85.90 |
| | GH57 | 168.33 | 95.83 | 54.76 | 123.84 | 66.95 | 127.41 |
| | GH59 | 21.86 | 16.74 | 8.98 | 0.00 | 20.26 | 0.00 |
| | GH63 | 157.26 | 130.86 | 121.25 | 42.55 | 66.53 | 190.54 |
| | GH64 | 15.06 | 0.00 | 0.00 | 0.00 | 0.00 | 0.00 |
| | GH65 | 48.84 | 21.58 | 58.58 | 23.44 | 6.01 | 93.23 |
| | GH66 | 23.23 | 53.30 | 24.82 | 0.00 | 0.00 | 54.86 |
| | GH67 | 25.64 | 49.57 | 40.75 | 144.03 | 5.92 | 72.89 |
| | GH68 | 0.00 | 0.00 | 0.00 | 0.00 | 0.00 | 0.00 |
| | GH70 | 0.00 | 0.00 | 0.00 | 0.00 | 0.00 | 0.00 |
| | GH73 | 373.39 | 305.12 | 454.03 | 580.88 | 307.63 | 375.31 |
| | GH74 | 144.87 | 26.61 | 50.75 | 0.00 | 42.11 | 43.36 |
| | GH76 | 134.18 | 5.01 | 55.16 | 0.00 | 16.44 | 279.90 |
| | GH77 | 258.41 | 367.78 | 361.01 | 321.22 | 337.41 | 278.43 |
| | GH78 | 285.20 | 319.79 | 184.00 | 228.17 | 86.71 | 691.66 |
| | GH79 | 0.00 | 0.00 | 8.58 | 8.76 | 0.00 | 0.00 |
| | GH8 | 38.01 | 62.08 | 98.95 | 146.65 | 82.10 | 32.25 |
| | GH81 | 0.00 | 0.00 | 0.00 | 0.00 | 0.00 | 0.00 |
| | GH84 | 174.03 | 110.68 | 107.13 | 154.75 | 34.51 | 104.35 |
| | GH85 | 13.43 | 5.68 | 14.71 | 0.00 | 0.00 | 21.96 |
| | GH87 | 13.48 | 0.00 | 0.00 | 0.00 | 0.00 | 0.00 |
| | GH88 | 304.11 | 50.35 | 225.90 | 104.93 | 84.62 | 490.48 |
| | GH89 | 151.90 | 93.61 | 49.51 | 48.64 | 25.35 | 290.09 |
| | GH9 | 218.08 | 0.00 | 220.26 | 5.97 | 11.69 | 162.70 |
| | GH91 | 21.39 | 0.00 | 0.00 | 0.00 | 0.00 | 22.09 |
| | GH92 | 863.05 | 210.10 | 429.95 | 190.02 | 262.16 | 1485.51 |
| | GH93 | 0.00 | 0.00 | 0.00 | 0.00 | 0.00 | 0.00 |
| | GH94 | 121.53 | 261.28 | 452.08 | 254.69 | 255.71 | 201.22 |
| | GH95 | 450.74 | 283.86 | 287.03 | 381.20 | 169.83 | 448.00 |
| | GH97 | 559.30 | 416.06 | 550.41 | 783.84 | 150.35 | 1099.76 |
| | GH98 | 24.73 | 14.00 | 0.00 | 8.10 | 0.00 | 22.69 |
| | GH99 | 0.00 | 0.00 | 0.00 | 0.00 | 0.00 | 21.48 |
| | PL1 | 166.40 | 159.40 | 131.19 | 152.06 | 14.98 | 305.78 |
| | PL10 | 59.64 | 19.52 | 62.88 | 21.99 | 14.79 | 143.60 |
| | PL11 | 53.57 | 19.42 | 67.57 | 26.56 | 34.68 | 198.12 |
| | PL12 | 54.66 | 4.92 | 80.56 | 30.48 | 30.65 | 70.9 |
| | PL13 | 24.20 | 0.00 | 41.25 | 0.00 | 0.00 | 27.11 |
| | PL15 | 22.80 | 0.00 | 82.91 | 0.00 | 0.00 | 88.00 |
| | PL16 | 8.19 | 0.00 | 0.00 | 0.00 | 0.00 | 0.00 |
| | PL17 | 0.00 | 0.00 | 7.43 | 0.00 | 9.65 | 33.29 |
| | PL21 | 22.04 | 0.00 | 21.00 | 0.00 | 0.00 | 0.00 |
| | PL22 | 44.26 | 9.10 | 33.84 | 31.60 | 7.63 | 48.38 |
| | PL26 | 20.69 | 8.05 | 22.67 | 11.10 | 15.77 | 72.50 |
| | PL27 | 20.12 | 12.57 | 18.35 | 0.00 | 0.00 | 80.53 |
| | PL29 | 5.00 | 0.00 | 0.00 | 0.00 | 15.56 | 29.14 |
| | PL30 | 23.93 | 0.00 | 23.73 | 0.00 | 6.53 | 48.91 |
| | PL31 | 15.14 | 0.00 | 18.42 | 0.00 | 8.51 | 0.00 |
| | PL33 | 69.62 | 22.34 | 33.50 | 0.00 | 23.85 | 55.52 |
| | PL34 | 0.00 | 0.00 | 0.00 | 0.00 | 7.49 | 0.00 |
| | PL35 | 37.59 | 5.34 | 26.24 | 0.00 | 2.93 | 59.39 |
| | PL4 | 0.00 | 0.00 | 0.00 | 7.32 | 0.00 | 0.00 |
| | PL6 | 0.00 | 9.23 | 9.08 | 0.00 | 0.00 | 23.36 |
| | PL7 | 0.00 | 0.00 | 0.00 | 0.00 | 0.00 | 0.00 |
| | PL8 | 113.37 | 4.86 | 65.57 | 11.39 | 10.84 | 134.03 |
| | PL9 | 71.92 | 0.00 | 112.64 | 25.72 | 7.33 | 82.67 |

TABLE S8D
Week 5. CAZyme (GH and PL) gene representation in the fecal microbiomes of participants
enrolled in open-label, single group assignment study consuming orange fiber snacks.

| | | Participant ID | | | | | | | |
|---|---|---|---|---|---|---|---|---|---|
| | | TP01.01 | TP01.02 | TP02.01 | TP02.02 | TP03.01 | TP03.02 | TP04.01 | TP04.02 |
| | | Timepoint | | | | | | | |
| | | Week 5 | Week 5 | Week 5 | Week 5 | Week 5 | Week 5 | Week 5 | Week 5 |
| | | Diet | | | | | | | |
| | | Orange | Orange | Orange | Orange | Orange | Orange | Orange | Orange |
| | | fiber (3 | fiber (3 | fiber (3 | fiber (3 | fiber (3 | fiber (3 | fiber (3 | fiber (3 |
| | | snacks/ | snacks/ | snacks/ | snacks/ | snacks/ | snacks/ | snacks/ | snacks/ |
| | | day) | day) | day) | day) | day) | day) | day) | day) |
| Read | GH1 | 327.88 | 439.05 | 94.51 | 343.36 | 148.70 | 242.92 | 355.00 | 260.55 |
| counts | GH10 | 93.15 | 188.81 | 147.03 | 96.42 | 154.72 | 46.49 | 120.97 | 49.95 |
| of | GH101 | 0.00 | 0.00 | 0.00 | 6.55 | 0.00 | 14.17 | 5.34 | 7.05 |
| CAZyme | GH102 | 0.00 | 0.00 | 5.09 | 0.00 | 0.00 | 0.00 | 0.00 | 0.00 |
| genes | GH103 | 0.00 | 0.00 | 0.00 | 0.00 | 0.00 | 0.00 | 0.00 | 0.00 |
| (TPM | GH105 | 216.15 | 422.04 | 856.21 | 539.25 | 507.72 | 705.17 | 401.42 | 767.28 |
| normal- | GH106 | 43.31 | 46.28 | 198.10 | 134.87 | 169.01 | 171.53 | 70.26 | 180.95 |
| ized) | GH108 | 27.20 | 24.86 | 1712.27 | 295.70 | 69.25 | 217.41 | 0.00 | 66.26 |
| | GH109 | 23.02 | 74.13 | 294.66 | 201.90 | 230.39 | 312.61 | 91.43 | 260.80 |
| | GH110 | 29.81 | 18.99 | 133.18 | 110.01 | 45.64 | 88.00 | 34.90 | 100.69 |
| | GH111 | 0.00 | 0.00 | 0.00 | 0.00 | 0.00 | 0.00 | 0.00 | 0.00 |
| | GH112 | 132.98 | 88.05 | 59.03 | 112.10 | 95.84 | 55.75 | 125.37 | 61.55 |
| | GH113 | 82.79 | 53.56 | 4.22 | 34.68 | 26.31 | 0.00 | 56.89 | 0.00 |
| | GH114 | 7.34 | 28.45 | 4.57 | 13.46 | 11.43 | 0.00 | 15.75 | 0.00 |
| | GH115 | 112.90 | 75.39 | 206.70 | 87.47 | 131.38 | 129.55 | 54.21 | 147.81 |
| | GH116 | 0.00 | 7.37 | 81.48 | 30.05 | 38.61 | 48.07 | 4.54 | 43.57 |
| | GH117 | 0.00 | 11.88 | 0.00 | 0.00 | 47.88 | 18.83 | 71.37 | 21.30 |
| | GH120 | 27.08 | 44.48 | 22.27 | 45.45 | 19.43 | 32.75 | 41.49 | 53.52 |
| | GH121 | 0.00 | 12.11 | 0.00 | 0.00 | 0.00 | 8.77 | 0.00 | 7.02 |
| | GH123 | 29.34 | 23.34 | 72.35 | 61.10 | 49.31 | 63.45 | 35.67 | 63.88 |
| | GH125 | 46.25 | 80.51 | 395.43 | 175.29 | 167.03 | 128.15 | 91.69 | 71.73 |
| | GH126 | 0.00 | 0.00 | 0.00 | 0.00 | 0.00 | 0.00 | 0.00 | 0.00 |
| | GH127 | 143.72 | 133.22 | 225.36 | 185.61 | 247.54 | 276.19 | 85.69 | 240.04 |
| | GH128 | 0.00 | 0.00 | 0.00 | 0.00 | 0.00 | 0.00 | 0.00 | 6.20 |
| | GH129 | 15.07 | 0.00 | 8.59 | 5.99 | 0.00 | 8.45 | 3.40 | 0.00 |
| | GH13 | 713.63 | 663.51 | 389.94 | 513.30 | 522.18 | 591.21 | 583.54 | 699.42 |
| | GH1311 | 50.33 | 166.28 | 77.96 | 83.13 | 70.33 | 164.34 | 67.98 | 179.07 |
| | GH1312 | 0.00 | 0.00 | 0.00 | 0.00 | 0.00 | 0.00 | 0.00 | 0.00 |
| | GH1314 | 203.41 | 134.50 | 151.38 | 208.09 | 214.01 | 108.18 | 158.39 | 83.45 |
| | GH1318 | 44.25 | 35.93 | 0.00 | 29.89 | 21.47 | 74.26 | 30.59 | 82.26 |
| | GH1319 | 7.26 | 12.41 | 0.00 | 13.48 | 0.00 | 0.00 | 0.00 | 0.00 |
| | GH132 | 0.00 | 0.00 | 0.00 | 0.00 | 0.00 | 0.00 | 0.00 | 0.00 |
| | GH1320 | 26.23 | 50.88 | 28.25 | 50.59 | 46.94 | 58.28 | 37.91 | 20.98 |
| | GH1328 | 4.85 | 27.71 | 0.00 | 30.19 | 0.00 | 0.00 | 26.09 | 0.00 |
| | GH1329 | 0.00 | 46.37 | 15.49 | 7.62 | 0.00 | 2.30 | 0.00 | 9.22 |
| | GH133 | 5.57 | 9.12 | 0.00 | 6.15 | 0.00 | 21.75 | 3.19 | 11.80 |
| | GH1330 | 8.65 | 14.76 | 0.00 | 6.18 | 0.00 | 27.95 | 11.01 | 4.24 |
| | GH1331 | 91.45 | 83.87 | 124.18 | 110.22 | 85.75 | 134.50 | 185.98 | 77.38 |
| | GH1332 | 3.03 | 17.17 | 0.00 | 0.00 | 0.00 | 25.55 | 0.00 | 2.05 |
| | GH1336 | 44.97 | 91.61 | 6.60 | 29.54 | 72.71 | 12.34 | 88.12 | 9.74 |
| | GH1337 | 0.00 | 13.99 | 0.00 | 0.00 | 0.00 | 0.00 | 0.00 | 0.00 |
| | GH1338 | 42.72 | 21.24 | 108.46 | 60.91 | 86.90 | 70.69 | 63.85 | 76.09 |
| | GH1339 | 117.28 | 59.88 | 1.70 | 77.31 | 82.66 | 50.32 | 136.55 | 5.31 |
| | GH134 | 40.16 | 95.82 | 43.41 | 58.70 | 65.99 | 3.83 | 171.26 | 17.74 |
| | GH1341 | 7.94 | 21.19 | 0.00 | 13.62 | 17.74 | 12.82 | 9.64 | 9.73 |
| | GH1342 | 0.00 | 0.00 | 0.00 | 0.00 | 0.00 | 0.00 | 0.00 | 0.00 |
| | GH135 | 34.79 | 47.26 | 12.53 | 0.00 | 0.00 | 0.00 | 50.64 | 0.00 |
| | GH138 | 45.81 | 28.66 | 108.49 | 52.88 | 87.75 | 63.02 | 54.29 | 76.09 |
| | GH139 | 423.11 | 465.31 | 107.61 | 374.51 | 235.29 | 325.24 | 446.69 | 383.43 |
| | GH130 | 378.71 | 289.60 | 442.18 | 352.86 | 385.54 | 189.57 | 311.79 | 197.36 |
| | GH133 | 49.38 | 65.69 | 148.30 | 80.56 | 132.95 | 68.77 | 73.83 | 90.51 |
| | GH136 | 29.38 | 27.98 | 77.15 | 51.35 | 44.09 | 80.72 | 14.48 | 66.00 |
| | GH137 | 43.06 | 10.51 | 57.18 | 34.11 | 27.76 | 34.87 | 0.00 | 52.81 |
| | GH138 | 46.74 | 11.69 | 37.47 | 26.91 | 29.96 | 34.68 | 6.44 | 51.83 |
| | GH139 | 0.00 | 13.35 | 49.27 | 36.57 | 31.10 | 62.90 | 7.20 | 53.31 |
| | GH140 | 47.38 | 22.22 | 105.64 | 67.84 | 57.70 | 123.36 | 10.10 | 118.65 |
| | GH141 | 83.88 | 24.55 | 132.30 | 55.56 | 76.72 | 111.27 | 9.93 | 109.65 |
| | GH142 | 5.06 | 11.84 | 56.91 | 34.77 | 29.21 | 60.23 | 0.00 | 59.84 |
| | GH143 | 11.60 | 36.66 | 67.94 | 28.85 | 37.48 | 89.36 | 0.00 | 67.38 |
| | GH144 | 46.31 | 38.05 | 258.05 | 101.82 | 143.13 | 54.37 | 117.75 | 66.71 |
| | GH145 | 5.83 | 24.37 | 140.66 | 63.63 | 75.43 | 88.15 | 12.74 | 87.77 |
| | GH146 | 15.91 | 74.88 | 196.34 | 97.34 | 213.40 | 180.13 | 134.93 | 164.02 |
| | GH147 | 0.00 | 0.00 | 13.46 | 5.26 | 43.63 | 3.28 | 0.00 | 7.54 |
| | GH148 | 0.00 | 0.00 | 0.00 | 2.87 | 6.15 | 0.00 | 0.00 | 0.00 |
| | GH15 | 0.00 | 19.26 | 60.50 | 52.72 | 21.23 | 63.46 | 4.25 | 45.33 |
| | GH151 | 3.37 | 0.00 | 21.96 | 0.00 | 7.49 | 0.00 | 0.00 | 3.64 |
| | GH153 | 0.00 | 0.00 | 0.00 | 0.00 | 0.00 | 0.00 | 0.00 | 0.00 |
| | GH154 | 32.24 | 39.86 | 374.33 | 150.01 | 211.37 | 141.76 | 37.17 | 219.30 |
| | GH155 | 0.00 | 8.09 | 432.49 | 47.26 | 66.33 | 15.80 | 26.35 | 71.83 |
| | GH156 | 0.00 | 0.00 | 0.00 | 5.51 | 0.00 | 0.00 | 0.00 | 0.00 |
| | GH157 | 0.00 | 0.00 | 0.00 | 0.00 | 0.00 | 0.00 | 0.00 | 0.00 |
| | GH158 | 0.00 | 9.68 | 70.66 | 28.67 | 24.81 | 13.81 | 0.00 | 11.77 |
| | GH159 | 0.00 | 3.40 | 0.00 | 0.00 | 4.33 | 0.00 | 0.00 | 4.30 |
| | GH16 | 146.75 | 113.89 | 277.11 | 172.06 | 207.28 | 134.48 | 233.45 | 208.93 |
| | GH161 | 6.40 | 0.00 | 0.00 | 9.30 | 0.00 | 0.00 | 0.00 | 0.00 |
| | GH17 | 0.00 | 0.00 | 0.00 | 0.00 | 8.84 | 0.00 | 0.00 | 0.00 |
| | GH18 | 82.74 | 203.75 | 896.34 | 247.19 | 245.62 | 289.43 | 366.94 | 354.82 |
| | GH19 | 0.00 | 0.00 | 0.00 | 0.00 | 0.00 | 0.00 | 0.00 | 0.00 |
| | GH2 | 1519.29 | 1121.61 | 2806.31 | 1905.34 | 2413.69 | 2044.77 | 1332.99 | 1957.89 |
| | GH20 | 315.95 | 240.09 | 962.69 | 675.28 | 653.53 | 806.10 | 532.07 | 767.53 |
| | GH23 | 419.22 | 470.83 | 535.19 | 555.26 | 480.42 | 497.52 | 488.97 | 488.80 |
| | GH24 | 38.32 | 106.97 | 417.29 | 112.03 | 352.91 | 195.22 | 35.68 | 208.49 |
| | GH25 | 1057.34 | 797.69 | 236.66 | 692.57 | 666.28 | 559.64 | 664.84 | 413.88 |
| | GH26 | 360.60 | 255.28 | 165.51 | 286.78 | 279.96 | 62.63 | 273.62 | 40.43 |
| | GH27 | 98.75 | 127.52 | 170.51 | 177.07 | 151.55 | 90.08 | 121.77 | 134.66 |
| | GH28 | 438.74 | 487.97 | 841.89 | 572.32 | 926.20 | 950.65 | 742.70 | 999.99 |
| | GH29 | 48.56 | 173.12 | 702.84 | 407.51 | 679.76 | 761.87 | 356.44 | 609.52 |
| | GH3 | 1027.73 | 825.49 | 2157.77 | 1216.80 | 1301.99 | 926.52 | 1154.82 | 756.95 |
| | GH30 | 0.00 | 4.14 | 0.00 | 0.00 | 3.76 | 0.00 | 0.00 | 0.00 |
| | GH301 | 3.2 | 25.72 | 0.00 | 3.76 | 4.60 | 2.98 | 0.00 | 4.41 |
| | GH302 | 4.80 | 12.37 | 5.55 | 0.00 | 0.00 | 16.63 | 7.50 | 16.62 |
| | GH303 | 40.45 | 14.88 | 95.91 | 32.61 | 24.89 | 19.97 | 0.00 | 16.81 |
| | GH304 | 40.94 | 42.39 | 183.42 | 86.59 | 34.83 | 156.31 | 34.62 | 138.18 |
| | GH305 | 2.88 | 0.00 | 0.00 | 7.06 | 0.00 | 9.30 | 5.58 | 0.00 |
| | GH306 | 6.94 | 13.56 | 43.09 | 34.49 | 10.84 | 74.05 | 4.82 | 72.69 |
| | GH308 | 0.00 | 13.18 | 28.78 | 42.98 | 5.88 | 0.00 | 22.75 | 4.14 |
| | GH31 | 450.12 | 466.27 | 842.34 | 631.65 | 587.33 | 516.86 | 331.90 | 521.48 |
| | GH32 | 317.15 | 388.77 | 506.11 | 332.54 | 366.90 | 318.39 | 358.99 | 285.60 |
| | GH33 | 119.00 | 101.36 | 210.36 | 176.10 | 250.24 | 240.27 | 96.74 | 265.86 |
| | GH35 | 66.48 | 35.36 | 257.51 | 112.63 | 242.83 | 89.13 | 100.23 | 109.73 |
| | GH36 | 470.09 | 422.73 | 550.30 | 431.17 | 450.14 | 259.47 | 453.64 | 233.41 |
| | GH37 | 0.00 | 0.00 | 0.00 | 0.00 | 0.00 | 0.00 | 0.00 | 0.00 |
| | GH38 | 79.08 | 73.87 | 225.84 | 138.08 | 100.79 | 87.93 | 46.21 | 113.16 |
| | GH39 | 36.39 | 42.64 | 12.05 | 39.51 | 28.55 | 3.34 | 31.16 | 16.77 |
| | GH4 | 50.84 | 38.70 | 36.80 | 64.29 | 32.36 | 153.25 | 80.33 | 476.68 |
| | GH42 | 138.09 | 166.04 | 239.41 | 148.50 | 120.54 | 171.70 | 191.95 | 118.93 |
| | GH43 | 0.00 | 33.53 | 196.94 | 53.14 | 48.64 | 47.98 | 25.83 | 41.06 |
| | GH431 | 47.86 | 13.62 | 43.75 | 40.22 | 50.29 | 32.07 | 62.92 | 25.80 |
| | GH4310 | 250.48 | 228.05 | 271.83 | 202.84 | 380.51 | 249.74 | 297.24 | 199.31 |
| | GH4311 | 34.22 | 45.96 | 25.84 | 35.51 | 17.18 | 21.94 | 37.41 | 9.83 |
| | GH4312 | 159.39 | 94.90 | 219.86 | 163.89 | 231.05 | 241.21 | 159.00 | 141.32 |
| | GH4316 | 12.35 | 103.75 | 56.32 | 52.21 | 133.04 | 34.76 | 91.44 | 23.12 |
| | GH4317 | 0.00 | 14.57 | 19.56 | 41.39 | 50.09 | 89.01 | 4.19 | 63.52 |
| | GH4318 | 4.90 | 22.46 | 147.75 | 70.14 | 78.83 | 121.69 | 10.86 | 129.21 |
| | GH4319 | 51.73 | 44.26 | 180.01 | 96.86 | 118.43 | 141.79 | 11.53 | 105.18 |
| | GH432 | 42.59 | 8.04 | 14.44 | 9.58 | 36.51 | 0.00 | 3.94 | 11.68 |
| | GH4322 | 16.70 | 15.71 | 4.54 | 8.11 | 6.47 | 48.39 | 23.05 | 7.29 |
| | GH4324 | 32.54 | 44.08 | 257.91 | 154.68 | 212.26 | 118.63 | 40.79 | 112.08 |
| | GH4326 | 18.17 | 75.50 | 55.91 | 38.08 | 39.37 | 32.57 | 29.18 | 27.67 |
| | GH4327 | 0.00 | 10.79 | 0.00 | 10.63 | 7.50 | 11.55 | 9.65 | 3.07 |
| | GH4328 | 37.35 | 22.80 | 110.43 | 35.67 | 66.81 | 53.51 | 34.41 | 26.35 |
| | GH4329 | 93.94 | 39.83 | 91.82 | 46.87 | 51.37 | 49.47 | 22.89 | 49.99 |
| | GH433 | 6.42 | 21.48 | 129.96 | 34.22 | 27.57 | 49.75 | 17.98 | 79.94 |
| | GH4330 | 0.00 | 0.00 | 0.00 | 4.99 | 0.00 | 0.00 | 0.00 | 0.00 |
| | GH4331 | 10.15 | 38.15 | 171.41 | 97.67 | 86.67 | 126.22 | 7.85 | 155.48 |
| | GH4332 | 0.00 | 3.04 | 0.00 | 0.00 | 4.46 | 0.00 | 5.41 | 0.00 |
| | GH4333 | 0.00 | 12.36 | 56.02 | 39.98 | 30.74 | 16.26 | 5.01 | 15.80 |
| | GH4334 | 55.78 | 51.29 | 310.28 | 179.42 | 146.58 | 234.44 | 12.23 | 166.61 |
| | GH4335 | 72.57 | 64.93 | 107.59 | 96.90 | 115.95 | 28.30 | 60.03 | 20.87 |
| | GH4337 | 0.00 | 0.00 | 0.00 | 0.00 | 0.00 | 0.00 | 0.00 | 0.00 |
| | GH434 | 45.39 | 51.77 | 67.81 | 106.14 | 93.16 | 158.74 | 53.33 | 156.74 |
| | GH435 | 45.41 | 0.00 | 0.00 | 0.00 | 0.00 | 0.00 | 0.00 | 0.00 |
| | GH437 | 36.93 | 0.00 | 0.00 | 0.00 | 32.12 | 0.00 | 0.00 | 5.82 |
| | GH438 | 0.00 | 0.00 | 12.73 | 9.81 | 30.23 | 0.00 | 0.00 | 0.00 |
| | GH439 | 5.64 | 23.28 | 48.76 | 64.81 | 58.21 | 109.57 | 0.00 | 17.85 |
| | GH44 | 0.00 | 13.18 | 0.00 | 16.66 | 0.00 | 0.00 | 20.38 | 0.00 |
| | GH48 | 0.00 | 16.19 | 0.00 | 24.68 | 0.00 | 6.10 | 9.95 | 0.00 |
| | GH5 | 117.11 | 72.35 | 3.68 | 92.21 | 6.35 | 6.07 | 49.43 | 2.93 |
| | GH51 | 49.54 | 15.63 | 0.00 | 50.80 | 48.81 | 3.74 | 26.89 | 0.00 |
| | GH513 | 0.00 | 0.00 | 60.07 | 9.87 | 15.42 | 0.00 | 14.09 | 4.93 |
| | GH518 | 0.00 | 0.00 | 0.00 | 5.47 | 0.00 | 0.00 | 0.00 | 4.22 |
| | GH52 | 143.02 | 223.00 | 103.91 | 119.44 | 100.42 | 52.45 | 138.24 | 36.23 |
| | GH521 | 41.72 | 0.00 | 6.30 | 3.80 | 0.00 | 0.00 | 8.25 | 6.17 |
| | GH522 | 0.00 | 0.00 | 0.00 | 0.00 | 0.00 | 0.00 | 0.00 | 0.00 |
| | GH525 | 0.00 | 0.00 | 0.00 | 0.00 | 0.00 | 17.10 | 0.00 | 0.00 |
| | GH526 | 0.00 | 0.00 | 0.00 | 0.00 | 0.00 | 0.00 | 0.00 | 13.27 |
| | GH527 | 0.00 | 0.00 | 0.00 | 0.00 | 0.00 | 2.81 | 4.42 | 0.00 |
| | GH537 | 81.44 | 72.15 | 0.00 | 40.95 | 53.70 | 0.00 | 65.94 | 0.00 |
| | GH538 | 0.00 | 0.00 | 16.94 | 3.34 | 0.00 | 0.00 | 0.00 | 0.00 |
| | GH54 | 279.15 | 258.46 | 197.84 | 366.86 | 311.25 | 62.71 | 228.59 | 29.02 |
| | GH544 | 5.1 | 11.26 | 0.00 | 7.90 | 0.00 | 32.29 | 15.75 | 14.58 |
| | GH546 | 2.05 | 12.16 | 68.97 | 31.81 | 33.17 | 16.51 | 0.00 | 13.30 |
| | GH55 | 0.00 | 0.00 | 52.39 | 11.14 | 5.60 | 0.00 | 6.44 | 3.87 |
| | GH57 | 84.98 | 42.81 | 58.74 | 84.48 | 111.81 | 20.33 | 40.14 | 14.23 |
| | GH58 | 48.44 | 14.12 | 6.41 | 42.54 | 155.11 | 0.00 | 43.47 | 0.00 |
| | GH50 | 0.00 | 0.00 | 97.32 | 5.2 | 15.68 | 0.00 | 10.44 | 6.19 |
| | GH51 | 307.79 | 213.24 | 467.47 | 373.08 | 250.92 | 317.65 | 125.96 | 315.32 |
| | GH53 | 133.11 | 226.18 | 134.99 | 141.62 | 175.24 | 132.03 | 192.97 | 103.96 |
| | GH55 | 0.00 | 33.98 | 108.99 | 55.17 | 79.90 | 25.48 | 0.00 | 39.60 |
| | GH57 | 45.20 | 23.07 | 127.51 | 57.87 | 98.03 | 68.29 | 88.13 | 83.05 |
| | GH59 | 58.43 | 53.70 | 0.00 | 65.28 | 4.37 | 3.96 | 44.34 | 0.00 |
| | GH63 | 27.92 | 60.33 | 213.09 | 180.90 | 228.29 | 178.68 | 64.27 | 146.63 |
| | GH64 | 0.00 | 0.00 | 0.00 | 0.00 | 0.00 | 0.00 | 0.00 | 0.00 |
| | GH65 | 0.00 | 22.21 | 104.47 | 56.25 | 31.26 | 39.74 | 77.78 | 61.09 |
| | GH66 | 0.00 | 17.73 | 94.42 | 36.93 | 37.62 | 14.67 | 10.14 | 18.47 |
| | GH67 | 51.94 | 12.27 | 42.77 | 33.02 | 60.64 | 27.05 | 53.25 | 27.57 |
| | GH68 | 0.00 | 0.00 | 0.00 | 0.00 | 0.00 | 0.00 | 0.00 | 0.00 |
| | GH70 | 0.00 | 0.00 | 0.00 | 0.00 | 0.00 | 0.00 | 0.00 | 0.00 |
| | GH73 | 234.35 | 283.02 | 216.03 | 297.02 | 337.91 | 310.64 | 329.56 | 481.36 |
| | GH74 | 102.31 | 52.42 | 61.96 | 148.32 | 87.93 | 16.71 | 57.52 | 13.62 |
| | GH76 | 4.09 | 33.51 | 554.52 | 152.92 | 123.78 | 96.42 | 38.99 | 72.80 |
| | GH77 | 460.84 | 417.10 | 245.50 | 418.86 | 360.18 | 384.90 | 515.93 | 340.84 |
| | GH78 | 175.99 | 163.01 | 546.95 | 290.02 | 232.14 | 311.50 | 128.70 | 261.64 |
| | GH79 | 0.00 | 0.00 | 0.00 | 0.00 | 0.00 | 0.00 | 0.00 | 0.00 |
| | GH8 | 92.73 | 73.53 | 5.71 | 13.93 | 61.00 | 31.46 | 47.49 | 18.91 |
| | GH81 | 0.00 | 0.00 | 0.00 | 0.00 | 0.00 | 0.00 | 0.00 | 5.88 |
| | GH84 | 54.90 | 22.25 | 95.12 | 56.91 | 137.34 | 71.63 | 18.32 | 74.80 |
| | GH85 | 0.00 | 0.00 | 4.09 | 20.18 | 39.70 | 4.40 | 7.37 | 5.04 |
| | GH87 | 0.00 | 0.00 | 0.00 | 0.00 | 0.00 | 0.00 | 0.00 | 0.00 |
| | GH88 | 45.83 | 147.92 | 553.63 | 254.09 | 343.16 | 222.18 | 360.20 | 302.26 |
| | GH89 | 11.17 | 26.30 | 222.87 | 87.54 | 119.45 | 101.80 | 153.05 | 208.66 |
| | GH9 | 113.80 | 246.22 | 302.25 | 329.74 | 141.33 | 94.99 | 234.54 | 71.83 |
| | GH91 | 0.00 | 4.11 | 82.20 | 4.69 | 17.99 | 3.29 | 3.21 | 21.40 |
| | GH92 | 132.77 | 195.69 | 1407.92 | 628.02 | 752.10 | 747.03 | 464.56 | 705.08 |
| | GH93 | 0.00 | 0.00 | 0.00 | 0.00 | 0.00 | 0.00 | 0.00 | 0.00 |
| | GH94 | 374.96 | 351.86 | 119.16 | 343.27 | 230.43 | 106.15 | 298.06 | 78.31 |
| | GH95 | 247.97 | 112.30 | 450.77 | 296.59 | 401.83 | 284.38 | 290.31 | 296.39 |
| | GH97 | 218.48 | 261.84 | 1132.93 | 555.16 | 630.81 | 566.56 | 257.33 | 602.15 |
| | GH98 | 0.00 | 2.60 | 43.36 | 0.00 | 0.00 | 3.65 | 0.00 | 0.00 |
| | GH99 | 0.00 | 0.00 | 36.73 | 0.00 | 14.53 | 19.54 | 6.35 | 0.00 |
| | PL1 | 350.33 | 145.59 | 305.00 | 178.51 | 118.48 | 171.61 | 296.58 | 209.68 |
| | PL10 | 6.55 | 49.10 | 86.60 | 73.18 | 68.79 | 138.42 | 83.57 | 136.63 |
| | PL11 | 36.48 | 106.11 | 178.00 | 142.97 | 128.55 | 230.21 | 178.70 | 191.72 |
| | PL12 | 0.00 | 12.01 | 154.95 | 43.23 | 23.64 | 9.16 | 180.71 | 92.40 |
| | PL13 | 0.00 | 12.25 | 100.82 | 29.82 | 5.00 | 0.00 | 63.50 | 6.13 |
| | PL15 | 0.00 | 11.67 | 165.12 | 35.49 | 18.38 | 2.53 | 73.38 | 9.47 |
| | PL16 | 0.00 | 5.18 | 0.00 | 4.31 | 5.68 | 0.00 | 0.00 | 0.00 |
| | PL17 | 0.00 | 0.00 | 4.80 | 6.81 | 5.71 | 9.33 | 0.00 | 0.00 |
| | PL21 | 0.00 | 0.00 | 0.00 | 0.00 | 0.00 | 0.00 | 104.59 | 6.48 |
| | PL22 | 0.00 | 12.77 | 12.95 | 23.88 | 24.07 | 55.27 | 0.00 | 103.52 |
| | PL26 | 0.00 | 14.31 | 60.42 | 30.10 | 37.14 | 54.15 | 4.60 | 50.99 |
| | PL27 | 6.00 | 18.45 | 40.05 | 29.40 | 7.79 | 41.72 | 2.47 | 16.88 |
| | PL29 | 0.00 | 0.00 | 57.77 | 7.00 | 11.46 | 29.55 | 8.84 | 48.77 |
| | PL30 | 0.00 | 10.87 | 0.00 | 0.00 | 26.81 | 15.83 | 51.42 | 20.30 |
| | PL31 | 6.98 | 0.00 | 0.00 | 31.15 | 0.00 | 0.00 | 7.79 | 0.00 |
| | PL33 | 27.36 | 33.43 | 79.65 | 14.05 | 51.40 | 17.97 | 88.24 | 14.32 |
| | PL34 | 0.00 | 0.00 | 0.00 | 0.00 | 8.64 | 16.60 | 0.00 | 0.00 |
| | PL35 | 0.00 | 9.46 | 75.25 | 18.88 | 18.75 | 4.62 | 5.66 | 14.57 |
| | PL4 | 0.00 | 3.52 | 0.00 | 0.00 | 0.00 | 0.00 | 0.00 | 0.00 |
| | PL6 | 0.00 | 0.00 | 5.95 | 6.15 | 6.59 | 3.65 | 0.00 | 0.00 |
| | PL7 | 0.00 | 0.00 | 0.00 | 0.00 | 0.00 | 6.67 | 0.00 | 0.00 |
| | PL8 | 4.71 | 17.97 | 270.03 | 76.69 | 57.58 | 16.83 | 190.62 | 84.89 |
| | PL9 | 144.85 | 230.73 | 147.17 | 130.33 | 129.02 | 189.58 | 164.68 | 161.53 |
| | | Participant ID | | | | | | | |
| | | TP05.01 | TP05.02 | TP06.01 | TP06.02 | TP07.01 | TP07.02 | TP08.01 | TP08.02 |
| | | Timepoint | | | | | | | |
| | | Week 5 | Week 5 | Week 5 | Week 5 | Week 5 | Week 5 | Week 5 | Week 5 |
| | | Diet | | | | | | | |
| | | Orange | Orange | Orange | Orange | Orange | Orange | Orange | Orange |
| | | fiber (3 | fiber (3 | fiber (3 | fiber (3 | fiber (3 | fiber (3 | fiber (3 | fiber (3 |
| | | snacks/ | snacks/ | snacks/ | snacks/ | snacks/ | snacks/ | snacks/ | snacks/ |
| | | day) | day) | day) | day) | day) | day) | day) | day) |
| Read | GH1 | 262.61 | 98.43 | 357.36 | 191.83 | 328.20 | 266.76 | 63.46 | 218.41 |
| counts | GH10 | 209.32 | 102.24 | 68.52 | 4.92 | 181.82 | 185.40 | 162.15 | 153.08 |
| of | GH101 | 0.00 | 12.86 | 7.24 | 0.00 | 9.26 | 12.46 | 2.44 | 0.00 |
| CAZyme | GH102 | 2.69 | 0.00 | 0.00 | 0.00 | 0.00 | 0.00 | 0.00 | 14.77 |
| genes | GH103 | 0.00 | 0.00 | 0.00 | 0.00 | 0.00 | 0.00 | 0.00 | 0.00 |
| (TPM | GH105 | 206.86 | 115.94 | 134.18 | 214.10 | 527.86 | 579.86 | 946.33 | 420.60 |
| normal- | GH106 | 153.66 | 39.65 | 59.69 | 64.02 | 200.58 | 215.43 | 345.26 | 103.41 |
| ized) | GH108 | 45.12 | 137.03 | 0.00 | 54.38 | 56.23 | 14.06 | 25.27 | 171.27 |
| | GH109 | 0.00 | 53.78 | 66.96 | 173.50 | 144.68 | 104.65 | 360.10 | 149.68 |
| | GH110 | 32.57 | 45.00 | 109.33 | 75.24 | 35.67 | 106.87 | 156.65 | 20.96 |
| | GH111 | 0.00 | 0.00 | 0.00 | 0.00 | 0.00 | 0.00 | 0.00 | 0.00 |
| | GH112 | 64.43 | 104.92 | 89.29 | 122.89 | 95.60 | 23.24 | 75.81 | 63.32 |
| | GH113 | 20.03 | 8.85 | 33.81 | 0.00 | 18.95 | 47.36 | 25.78 | 33.42 |
| | GH114 | 11.59 | 37.48 | 8.98 | 0.00 | 0.00 | 0.00 | 0.00 | 0.00 |
| | GH115 | 174.62 | 35.63 | 87.47 | 33.26 | 182.37 | 124.59 | 302.71 | 110.45 |
| | GH116 | 0.00 | 13.32 | 0.00 | 0.00 | 23.42 | 35.30 | 59.27 | 90.22 |
| | GH117 | 0.00 | 2.78 | 0.00 | 29.61 | 28.56 | 33.69 | 15.03 | 47.84 |
| | GH120 | 23.47 | 40.74 | 51.64 | 39.19 | 46.85 | 18.30 | 62.00 | 37.75 |
| | GH121 | 0.00 | 14.08 | 0.00 | 0.00 | 9.49 | 13.63 | 0.00 | 0.00 |
| | GH123 | 29.07 | 55.20 | 66.27 | 122.00 | 30.35 | 76.63 | 119.89 | 24.29 |
| | GH125 | 71.32 | 49.83 | 44.09 | 147.25 | 122.16 | 113.70 | 174.18 | 247.36 |
| | GH126 | 0.00 | 0.00 | 0.00 | 0.00 | 0.00 | 0.00 | 0.00 | 0.00 |
| | GH127 | 278.64 | 103.21 | 173.58 | 102.18 | 194.31 | 164.12 | 275.41 | 172.23 |
| | GH128 | 0.00 | 16.60 | 11.50 | 0.00 | 0.00 | 0.00 | 0.00 | 0.00 |
| | GH129 | 0.00 | 24.37 | 13.65 | 8.83 | 11.66 | 28.65 | 0.00 | 4.23 |
| | GH13 | 793.64 | 480.61 | 484.42 | 310.59 | 621.06 | 515.69 | 576.86 | 543.87 |
| | GH1311 | 25.83 | 125.96 | 95.79 | 69.10 | 79.75 | 68.55 | 75.32 | 123.74 |
| | GH1312 | 4.44 | 0.00 | 0.00 | 0.00 | 0.00 | 0.00 | 0.00 | 5.81 |
| | GH1314 | 200.15 | 143.89 | 280.58 | 133.91 | 143.10 | 149.18 | 179.85 | 118.75 |
| | GH1318 | 14.28 | 31.79 | 58.74 | 90.93 | 12.33 | 3.83 | 24.56 | 22.85 |
| | GH1319 | 15.54 | 21.54 | 40.10 | 23.86 | 0.00 | 15.98 | 0.00 | 0.00 |
| | GH132 | 7.14 | 0.00 | 4.25 | 5.44 | 0.00 | 0.00 | 0.00 | 0.00 |
| | GH1320 | 9.24 | 40.64 | 50.55 | 74.94 | 41.48 | 15.23 | 38.49 | 37.63 |
| | GH1328 | 13.47 | 50.63 | 98.55 | 36.18 | 3.49 | 36.19 | 7.60 | 13.33 |
| | GH1329 | 0.00 | 0.00 | 26.90 | 9.18 | 4.44 | 12.33 | 6.66 | 7.49 |
| | GH133 | 0.00 | 14.33 | 18.60 | 9.00 | 8.33 | 12.33 | 0.00 | 0.00 |
| | GH1330 | 0.00 | 32.68 | 53.61 | 15.76 | 7.86 | 23.78 | 0.00 | 0.00 |
| | GH1331 | 61.65 | 163.22 | 198.50 | 126.48 | 154.19 | 73.77 | 77.65 | 100.73 |
| | GH1332 | 0.00 | 0.00 | 36.93 | 17.09 | 0.00 | 0.00 | 0.00 | 5.40 |
| | GH1336 | 27.90 | 51.62 | 48.38 | 30.79 | 62.83 | 49.58 | 21.78 | 26.48 |
| | GH1337 | 0.00 | 0.00 | 0.00 | 0.00 | 0.00 | 0.00 | 0.00 | 0.00 |
| | GH1338 | 79.43 | 18.32 | 41.36 | 44.13 | 58.49 | 31.80 | 130.52 | 57.73 |
| | GH1339 | 121.13 | 168.89 | 186.40 | 185.35 | 46.87 | 140.18 | 21.77 | 24.56 |
| | GH134 | 71.57 | 48.95 | 26.05 | 38.93 | 114.84 | 24.57 | 18.01 | 116.88 |
| | GH1341 | 7.65 | 4.77 | 5.18 | 10.10 | 21.13 | 0.00 | 22.04 | 18.96 |
| | GH1342 | 0.00 | 0.00 | 0.00 | 0.00 | 0.00 | 0.00 | 4.06 | 0.00 |
| | GH135 | 32.37 | 23.29 | 0.00 | 26.85 | 16.08 | 30.32 | 28.20 | 6.48 |
| | GH138 | 90.22 | 28.91 | 50.48 | 41.76 | 47.12 | 27.99 | 115.41 | 48.82 |
| | GH139 | 316.49 | 380.25 | 343.28 | 367.27 | 255.23 | 275.93 | 197.82 | 331.42 |
| | GH130 | 305.55 | 126.64 | 199.98 | 236.57 | 244.82 | 325.04 | 410.82 | 239.84 |
| | GH133 | 108.90 | 27.30 | 67.61 | 48.11 | 73.21 | 69.39 | 147.63 | 77.12 |
| | GH136 | 0.00 | 21.01 | 15.25 | 21.27 | 76.16 | 74.86 | 95.18 | 36.86 |
| | GH137 | 28.27 | 0.00 | 30.30 | 0.00 | 0.00 | 26.90 | 88.15 | 13.81 |
| | GH138 | 30.85 | 0.00 | 28.09 | 0.00 | 5.78 | 27.13 | 121.59 | 13.96 |
| | GH139 | 0.00 | 0.00 | 0.00 | 5.54 | 43.03 | 40.49 | 87.57 | 19.35 |
| | GH140 | 35.29 | 5.78 | 28.13 | 11.52 | 71.34 | 74.04 | 173.27 | 50.52 |
| | GH141 | 74.29 | 0.00 | 24.21 | 20.86 | 85.30 | 134.04 | 163.34 | 60.52 |
| | GH142 | 0.00 | 0.00 | 5.71 | 7.88 | 34.49 | 33.07 | 108.70 | 21.03 |
| | GH143 | 0.00 | 0.00 | 0.00 | 0.00 | 28.35 | 26.21 | 131.04 | 21.03 |
| | GH144 | 1.75 | 3.92 | 0.00 | 83.46 | 115.95 | 139.86 | 126.86 | 138.42 |
| | GH145 | 0.00 | 0.00 | 0.00 | 32.57 | 67.53 | 32.55 | 104.95 | 70.01 |
| | GH146 | 6.43 | 22.46 | 7.93 | 64.29 | 203.89 | 116.72 | 231.68 | 159.92 |
| | GH147 | 0.00 | 6.72 | 0.00 | 0.00 | 6.10 | 17.15 | 52.34 | 0.00 |
| | GH148 | 0.00 | 0.00 | 0.00 | 0.00 | 0.00 | 0.00 | 3.56 | 0.00 |
| | GH15 | 5.83 | 12.44 | 0.00 | 19.43 | 30.53 | 0.00 | 74.45 | 38.79 |
| | GH151 | 15.26 | 0.00 | 6.57 | 0.00 | 6.49 | 0.00 | 0.00 | 0.00 |
| | GH153 | 0.00 | 0.00 | 0.00 | 4.16 | 0.00 | 0.00 | 0.00 | 0.00 |
| | GH154 | 114.74 | 76.08 | 41.29 | 40.61 | 122.37 | 136.58 | 223.68 | 140.27 |
| | GH155 | 0.00 | 7.09 | 0.00 | 0.00 | 27.94 | 85.03 | 125.32 | 30.13 |
| | GH156 | 0.00 | 0.00 | 0.00 | 0.00 | 10.61 | 0.00 | 9.71 | 0.00 |
| | GH157 | 0.00 | 0.00 | 0.00 | 0.00 | 8.07 | 17.00 | 0.00 | 0.00 |
| | GH158 | 0.00 | 0.00 | 0.00 | 24.66 | 20.70 | 10.40 | 0.00 | 40.03 |
| | GH159 | 0.00 | 14.58 | 5.29 | 0.00 | 14.38 | 41.43 | 8.89 | 0.00 |
| | GH16 | 187.37 | 177.05 | 88.74 | 97.32 | 197.15 | 182.34 | 279.96 | 157.81 |
| | GH161 | 0.00 | 8.02 | 0.00 | 0.00 | 0.00 | 0.00 | 0.00 | 0.00 |
| | GH17 | 0.00 | 0.00 | 0.00 | 0.00 | 0.00 | 0.00 | 0.00 | 0.00 |
| | GH18 | 78.19 | 261.28 | 78.20 | 199.27 | 288.67 | 372.59 | 466.89 | 438.80 |
| | GH19 | 11.17 | 0.00 | 0.00 | 0.00 | 0.00 | 0.00 | 0.00 | 0.00 |
| | GH2 | 1666.68 | 735.42 | 1077.91 | 1380.99 | 1796.92 | 1913.77 | 3102.52 | 1921.36 |
| | GH20 | 312.12 | 389.12 | 201.38 | 446.22 | 494.71 | 599.65 | 1097.71 | 486.00 |
| | GH23 | 457.43 | 376.78 | 458.26 | 590.50 | 474.37 | 412.53 | 506.01 | 458.00 |
| | GH24 | 87.54 | 127.15 | 103.63 | 201.13 | 222.35 | 131.36 | 251.97 | 160.13 |
| | GH25 | 902.43 | 624.02 | 568.76 | 549.67 | 484.45 | 683.74 | 443.48 | 674.15 |
| | GH26 | 285.74 | 183.14 | 100.38 | 75.25 | 177.27 | 447.50 | 193.65 | 193.45 |
| | GH27 | 82.75 | 146.55 | 83.14 | 126.41 | 143.36 | 232.84 | 122.48 | 108.54 |
| | GH28 | 627.92 | 169.53 | 279.16 | 502.06 | 873.54 | 762.50 | 1304.66 | 608.59 |
| | GH29 | 98.81 | 177.54 | 264.04 | 471.36 | 404.99 | 374.48 | 936.36 | 288.07 |
| | GH3 | 921.52 | 891.27 | 790.44 | 1221.37 | 1223.74 | 1418.34 | 1650.53 | 1251.52 |
| | GH30 | 0.00 | 12.34 | 0.00 | 0.00 | 5.18 | 0.00 | 23.37 | 0.00 |
| | GH301 | 0.00 | 21.51 | 5.86 | 0.00 | 12.51 | 0.00 | 0.00 | 10.29 |
| | GH302 | 0.00 | 4.03 | 12.64 | 6.47 | 42.25 | 73.06 | 0.00 | 15.53 |
| | GH303 | 29.64 | 7.22 | 28.11 | 31.90 | 52.62 | 63.53 | 37.71 | 37.44 |
| | GH304 | 66.11 | 0.00 | 122.09 | 117.82 | 109.65 | 84.21 | 182.80 | 88.68 |
| | GH305 | 0.00 | 14.84 | 0.00 | 0.00 | 12.99 | 14.77 | 2.77 | 0.00 |
| | GH306 | 0.00 | 0.00 | 0.00 | 0.00 | 11.82 | 0.00 | 56.36 | 0.00 |
| | GH308 | 7.16 | 0.00 | 12.74 | 0.00 | 12.74 | 33.65 | 0.00 | 0.00 |
| | GH31 | 409.84 | 323.37 | 304.47 | 410.48 | 607.50 | 527.61 | 776.76 | 512.40 |
| | GH32 | 301.58 | 211.15 | 314.49 | 361.77 | 427.38 | 249.80 | 262.15 | 448.05 |
| | GH33 | 107.46 | 95.22 | 242.77 | 239.38 | 186.14 | 151.88 | 338.18 | 115.24 |
| | GH35 | 104.78 | 65.43 | 69.20 | 92.88 | 59.26 | 138.10 | 248.04 | 70.71 |
| | GH36 | 484.46 | 327.70 | 338.13 | 267.84 | 366.08 | 444.32 | 491.04 | 364.40 |
| | GH37 | 0.00 | 0.00 | 0.00 | 11.46 | 0.00 | 10.82 | 0.00 | 0.00 |
| | GH38 | 25.78 | 73.94 | 134.63 | 218.86 | 68.79 | 202.67 | 163.76 | 65.31 |
| | GH39 | 19.83 | 42.17 | 68.08 | 32.97 | 26.54 | 21.28 | 69.90 | 25.02 |
| | GH4 | 38.10 | 26.95 | 124.75 | 129.65 | 8.22 | 40.42 | 24.24 | 12.29 |
| | GH42 | 64.14 | 121.05 | 161.79 | 125.79 | 191.60 | 144.00 | 183.82 | 176.17 |
| | GH43 | 10.11 | 20.08 | 37.63 | 0.00 | 38.02 | 54.84 | 26.28 | 87.07 |
| | GH431 | 105.46 | 0.00 | 28.49 | 7.89 | 41.15 | 30.32 | 102.57 | 11.43 |
| | GH4310 | 283.52 | 159.65 | 178.55 | 105.26 | 347.28 | 250.93 | 391.63 | 218.06 |
| | GH4311 | 7.61 | 70.26 | 110.55 | 15.69 | 28.97 | 23.40 | 20.39 | 35.28 |
| | GH4312 | 92.42 | 68.30 | 89.38 | 87.76 | 183.48 | 165.18 | 409.79 | 138.03 |
| | GH4316 | 11.55 | 133.51 | 5.60 | 23.93 | 118.49 | 160.10 | 44.20 | 86.96 |
| | GH4317 | 0.00 | 7.17 | 64.62 | 24.62 | 48.36 | 37.30 | 76.59 | 20.66 |
| | GH4318 | 4.06 | 0.00 | 0.00 | 9.48 | 88.26 | 59.46 | 197.24 | 42.57 |
| | GH4319 | 97.11 | 28.21 | 30.38 | 28.67 | 101.58 | 63.33 | 159.18 | 86.88 |
| | GH432 | 94.40 | 0.00 | 31.05 | 0.00 | 24.52 | 51.47 | 109.60 | 0.00 |
| | GH4322 | 0.00 | 46.65 | 43.51 | 31.08 | 12.64 | 13.89 | 14.10 | 2.44 |
| | GH4324 | 94.69 | 82.07 | 12.99 | 77.23 | 171.06 | 102.18 | 258.13 | 132.51 |
| | GH4326 | 11.30 | 54.10 | 29.19 | 21.72 | 57.56 | 61.21 | 97.93 | 75.30 |
| | GH4327 | 0.00 | 21.45 | 15.48 | 8.5 | 2.76 | 11.27 | 0.00 | 0.00 |
| | GH4328 | 16.71 | 18.84 | 8.98 | 63.67 | 100.28 | 106.72 | 167.57 | 100.19 |
| | GH4329 | 174.09 | 14.96 | 65.63 | 8.2 | 83.04 | 113.15 | 250.90 | 38.32 |
| | GH433 | 0.00 | 13.74 | 0.00 | 7.00 | 90.79 | 75.68 | 150.53 | 71.64 |
| | GH4330 | 0.00 | 0.00 | 0.00 | 0.00 | 5.74 | 0.00 | 0.00 | 0.00 |
| | GH4331 | 3.24 | 0.00 | 0.00 | 22.98 | 115.40 | 38.93 | 184.09 | 158.91 |
| | GH4332 | 5.90 | 20.18 | 0.00 | 17.21 | 0.00 | 18.85 | 4.42 | 0.00 |
| | GH4333 | 0.00 | 17.88 | 0.00 | 32.96 | 19.84 | 4.05 | 27.37 | 28.21 |
| | GH4334 | 82.78 | 20.37 | 31.34 | 29.51 | 180.95 | 137.48 | 265.57 | 138.00 |
| | GH4335 | 58.36 | 115.11 | 39.33 | 77.60 | 64.65 | 84.50 | 96.07 | 88.68 |
| | GH4337 | 0.00 | 0.00 | 0.00 | 0.00 | 3.58 | 0.00 | 0.00 | 0.00 |
| | GH434 | 134.28 | 36.90 | 33.31 | 19.12 | 141.35 | 70.02 | 195.97 | 123.93 |
| | GH435 | 88.00 | 0.00 | 29.88 | 0.00 | 3.75 | 0.00 | 0.00 | 9.84 |
| | GH437 | 78.45 | 0.00 | 29.19 | 0.00 | 5.31 | 18.56 | 31.16 | 0.00 |
| | GH438 | 0.00 | 0.00 | 0.00 | 0.00 | 16.29 | 9.53 | 20.95 | 13.60 |
| | GH439 | 0.00 | 0.00 | 0.00 | 6.26 | 67.44 | 15.88 | 104.24 | 31.61 |
| | GH44 | 43.44 | 0.00 | 0.00 | 0.00 | 0.00 | 14.68 | 0.00 | 0.00 |
| | GH48 | 9.57 | 0.00 | 6.31 | 0.00 | 0.00 | 18.32 | 0.00 | 0.00 |
| | GH5 | 96.94 | 5.55 | 139.90 | 99.95 | 38.93 | 56.87 | 24.37 | 51.30 |
| | GH51 | 52.72 | 31.90 | 11.38 | 0.00 | 9.97 | 43.55 | 23.49 | 38.47 |
| | GH513 | 9.76 | 7.90 | 0.00 | 6.04 | 0.00 | 23.23 | 57.81 | 12.99 |
| | GH518 | 0.00 | 0.00 | 0.00 | 0.00 | 6.87 | 10.49 | 0.00 | 0.00 |
| | GH52 | 181.89 | 123.52 | 26.55 | 71.61 | 152.17 | 100.59 | 73.46 | 168.30 |
| | GH521 | 83.71 | 0.00 | 28.36 | 0.00 | 7.30 | 48.60 | 51.92 | 5.12 |
| | GH522 | 0.00 | 4.45 | 0.00 | 0.00 | 0.00 | 0.00 | 0.00 | 0.00 |
| | GH525 | 0.00 | 0.00 | 0.00 | 0.00 | 0.00 | 0.00 | 0.00 | 0.00 |
| | GH526 | 0.00 | 0.00 | 0.00 | 0.00 | 0.00 | 0.00 | 0.00 | 0.00 |
| | GH527 | 0.00 | 0.00 | 5.78 | 12.74 | 0.00 | 0.00 | 0.00 | 0.00 |
| | GH537 | 45.78 | 77.58 | 14.70 | 0.00 | 19.26 | 57.65 | 20.75 | 38.88 |
| | GH538 | 0.00 | 0.00 | 0.00 | 0.00 | 3.45 | 0.00 | 6.38 | 0.00 |
| | GH54 | 298.51 | 281.48 | 151.36 | 58.15 | 169.74 | 366.44 | 168.99 | 319.83 |
| | GH544 | 0.00 | 18.73 | 26.71 | 15.61 | 7.93 | 11.81 | 0.00 | 3.82 |
| | GH546 | 0.00 | 3.74 | 0.00 | 30.38 | 32.76 | 23.87 | 18.19 | 39.75 |
| | GH55 | 0.00 | 0.00 | 0.00 | 0.00 | 9.51 | 25.80 | 46.74 | 12.94 |
| | GH57 | 99.10 | 33.35 | 35.04 | 25.62 | 39.75 | 108.19 | 72.94 | 60.84 |
| | GH58 | 22.14 | 68.44 | 18.72 | 0.00 | 5.77 | 73.23 | 19.04 | 29.82 |
| | GH50 | 2.28 | 4.97 | 0.00 | 78.20 | 42.99 | 81.49 | 63.56 | 25.01 |
| | GH51 | 438.20 | 192.56 | 228.82 | 241.18 | 345.60 | 281.30 | 509.79 | 347.34 |
| | GH53 | 181.83 | 44.95 | 72.63 | 76.71 | 148.72 | 55.70 | 160.27 | 150.09 |
| | GH55 | 8.78 | 0.00 | 0.00 | 136.32 | 37.11 | 0.00 | 43.09 | 79.55 |
| | GH57 | 98.30 | 57.45 | 55.95 | 59.54 | 67.78 | 66.42 | 95.51 | 63.00 |
| | GH59 | 29.35 | 41.22 | 12.17 | 0.00 | 24.25 | 43.20 | 33.41 | 52.78 |
| | GH63 | 39.88 | 34.52 | 65.54 | 127.98 | 150.05 | 110.27 | 215.00 | 168.47 |
| | GH64 | 0.00 | 0.00 | 0.00 | 0.00 | 0.00 | 0.00 | 0.00 | 0.00 |
| | GH65 | 0.00 | 30.58 | 16.22 | 46.13 | 35.02 | 69.71 | 121.35 | 56.82 |
| | GH66 | 8.62 | 18.35 | 0.00 | 32.71 | 29.70 | 45.10 | 46.42 | 41.77 |
| | GH67 | 90.17 | 4.86 | 29.98 | 12.50 | 45.67 | 47.47 | 122.07 | 16.26 |
| | GH68 | 6.22 | 0.00 | 0.00 | 0.00 | 0.00 | 0.00 | 0.00 | 6.56 |
| | GH70 | 9.89 | 0.00 | 0.00 | 0.00 | 0.00 | 0.00 | 0.00 | 17.30 |
| | GH73 | 515.24 | 357.26 | 288.20 | 334.88 | 324.89 | 210.45 | 391.70 | 372.47 |
| | GH74 | 51.58 | 46.80 | 28.86 | 26.61 | 49.26 | 62.01 | 54.27 | 89.09 |
| | GH76 | 0.00 | 11.62 | 0.00 | 88.73 | 115.03 | 124.48 | 291.15 | 170.34 |
| | GH77 | 377.53 | 358.76 | 377.64 | 409.67 | 337.49 | 312.48 | 273.96 | 333.97 |
| | GH78 | 146.11 | 182.62 | 322.63 | 273.12 | 315.32 | 436.93 | 652.47 | 189.99 |
| | GH79 | 0.00 | 0.00 | 0.00 | 0.00 | 7.22 | 16.98 | 0.00 | 4.47 |
| | GH8 | 104.53 | 92.84 | 79.02 | 48.43 | 32.15 | 39.28 | 128.72 | 42.04 |
| | GH81 | 0.00 | 30.70 | 23.20 | 0.00 | 0.00 | 0.00 | 0.00 | 0.00 |
| | GH84 | 0.00 | 26.45 | 31.25 | 42.41 | 50.59 | 10.61 | 90.52 | 57.11 |
| | GH85 | 0.00 | 28.51 | 10.51 | 0.00 | 0.00 | 9.93 | 13.84 | 0.00 |
| | GH87 | 0.00 | 0.00 | 0.00 | 0.00 | 0.00 | 0.00 | 0.00 | 0.00 |
| | GH88 | 35.46 | 9.79 | 43.00 | 144.93 | 269.25 | 269.26 | 631.36 | 268.57 |
| | GH89 | 2.83 | 32.24 | 89.01 | 73.53 | 87.95 | 67.38 | 228.14 | 65.78 |
| | GH9 | 87.23 | 55.65 | 18.31 | 110.15 | 186.59 | 178.17 | 82.46 | 165.48 |
| | GH91 | 0.00 | 0.00 | 0.00 | 0.00 | 0.00 | 0.00 | 16.34 | 12.26 |
| | GH92 | 99.18 | 113.97 | 94.89 | 277.50 | 600.97 | 603.55 | 1422.87 | 588.13 |
| | GH93 | 0.00 | 0.00 | 0.00 | 0.00 | 0.00 | 17.53 | 0.00 | 0.00 |
| | GH94 | 359.14 | 295.27 | 259.32 | 155.09 | 262.85 | 157.67 | 202.58 | 305.76 |
| | GH95 | 354.86 | 128.36 | 129.02 | 166.72 | 312.94 | 384.93 | 618.91 | 257.86 |
| | GH97 | 491.49 | 46.65 | 231.75 | 434.66 | 515.05 | 496.34 | 1011.60 | 588.12 |
| | GH98 | 11.80 | 0.00 | 15.30 | 0.00 | 5.97 | 38.04 | 0.00 | 0.00 |
| | GH99 | 0.00 | 3.39 | 0.00 | 0.00 | 12.89 | 19.35 | 82.39 | 7.41 |
| | PL1 | 165.13 | 8.27 | 75.56 | 11.49 | 444.16 | 270.64 | 390.53 | 119.10 |
| | PL10 | 0.00 | 0.00 | 0.00 | 15.86 | 75.41 | 80.50 | 165.54 | 49.19 |
| | PL11 | 51.15 | 12.34 | 0.00 | 24.40 | 157.37 | 138.63 | 282.14 | 84.36 |
| | PL12 | 4.80 | 0.00 | 12.23 | 27.49 | 44.61 | 39.41 | 120.46 | 43.80 |
| | PL13 | 0.00 | 0.00 | 0.00 | 23.77 | 36.87 | 31.13 | 71.53 | 21.10 |
| | PL15 | 0.00 | 0.00 | 0.00 | 23.18 | 35.16 | 54.27 | 220.41 | 26.41 |
| | PL16 | 8.50 | 0.00 | 9.01 | 0.00 | 12.47 | 0.00 | 0.00 | 4.94 |
| | PL17 | 0.00 | 0.00 | 0.00 | 0.00 | 0.00 | 31.04 | 75.31 | 6.26 |
| | PL21 | 0.00 | 0.00 | 0.00 | 0.00 | 0.00 | 0.00 | 58.90 | 0.00 |
| | PL22 | 0.00 | 0.00 | 3.57 | 6.57 | 21.62 | 0.00 | 50.76 | 13.84 |
| | PL26 | 0.00 | 0.00 | 0.00 | 5.82 | 47.25 | 30.16 | 64.52 | 23.32 |
| | PL27 | 0.00 | 4.65 | 0.00 | 6.89 | 18.26 | 17.46 | 65.69 | 0.00 |
| | PL29 | 0.00 | 0.00 | 0.00 | 0.00 | 0.00 | 12.26 | 80.83 | 9.87 |
| | PL30 | 0.00 | 0.00 | 0.00 | 25.26 | 0.00 | 25.88 | 39.75 | 33.00 |
| | PL31 | 0.00 | 0.00 | 4.23 | 0.00 | 2.62 | 11.01 | 0.00 | 7.47 |
| | PL33 | 22.48 | 0.00 | 29.69 | 10.11 | 53.51 | 73.34 | 66.85 | 42.23 |
| | PL34 | 0.00 | 0.00 | 0.00 | 0.00 | 0.00 | 0.00 | 17.87 | 4.20 |
| | PL35 | 0.00 | 0.00 | 4.68 | 0.00 | 16.78 | 27.06 | 104.24 | 30.95 |
| | PL4 | 0.00 | 0.00 | 0.00 | 0.00 | 0.00 | 0.00 | 0.00 | 0.00 |
| | PL6 | 0.00 | 0.00 | 0.00 | 0.00 | 6.31 | 28.47 | 40.56 | 7.62 |
| | PL7 | 8.80 | 0.00 | 0.00 | 0.00 | 0.00 | 0.00 | 15.00 | 0.00 |
| | PL8 | 11.36 | 0.00 | 0.00 | 22.64 | 100.80 | 150.47 | 154.71 | 74.15 |
| | PL9 | 70.99 | 32.04 | 0.00 | 5.97 | 418.90 | 141.86 | 75.85 | 196.30 |

| | | Participant ID | | | | | |
|---|---|---|---|---|---|---|---|
| | | TP09.01 | TP09.02 | TP10.02 | TP11.01 | TP12.01 | TP12.02 |
| | | Timepoint | | | | | |
| | | Week 5 | Week 5 | Week 5 | Week 5 | Week 5 | Week 5 |
| | | Diet | | | | | |
| | | Orange | Orange | Orange | Orange | Orange | Orange |
| | | fiber (3 | fiber (3 | fiber (3 | fiber (3 | fiber (3 | fiber (3 |
| | | snacks/ | snacks/ | snacks/ | snacks/ | snacks/ | snacks/ |
| | | day) | day) | day) | day) | day) | day) |
| Read | GH1 | 238.85 | 404.00 | 189.27 | 95.65 | 369.81 | 221.19 |
| counts | GH10 | 121.65 | 112.45 | 196.58 | 220.80 | 89.46 | 114.60 |
| of | GH101 | 62.95 | 0.00 | 10.34 | 0.00 | 0.00 | 3.03 |
| CAZyme | GH102 | 13.65 | 9.30 | 0.00 | 0.00 | 0.00 | 0.00 |
| genes | GH103 | 0.00 | 0.00 | 0.00 | 0.00 | 0.00 | 0.00 |
| (TPM | GH105 | 618.35 | 181.96 | 495.38 | 220.28 | 241.69 | 496.06 |
| normal- | GH106 | 206.64 | 102.77 | 157.07 | 227.75 | 9.20 | 210.14 |
| ized) | GH108 | 26.15 | 28.02 | 299.63 | 10.12 | 41.81 | 765.56 |
| | GH109 | 379.10 | 38.79 | 143.19 | 0.00 | 37.56 | 224.91 |
| | GH110 | 146.30 | 61.71 | 57.13 | 0.00 | 38.48 | 109.62 |
| | GH111 | 7.80 | 0.00 | 8.32 | 0.00 | 0.00 | 7.10 |
| | GH112 | 118.23 | 143.91 | 107.54 | 45.39 | 167.81 | 96.55 |
| | GH113 | 0.00 | 12.33 | 5.54 | 9.80 | 37.38 | 11.74 |
| | GH114 | 10.18 | 0.00 | 0.00 | 7.13 | 22.78 | 10.63 |
| | GH115 | 150.09 | 136.73 | 292.33 | 482.40 | 64.01 | 124.53 |
| | GH116 | 50.49 | 3.51 | 141.95 | 0.00 | 30.03 | 72.24 |
| | GH117 | 19.04 | 0.00 | 18.59 | 0.00 | 0.00 | 47.26 |
| | GH120 | 35.49 | 22.11 | 80.57 | 33.54 | 30.33 | 60.41 |
| | GH121 | 0.00 | 0.00 | 0.00 | 0.00 | 0.00 | 10.09 |
| | GH123 | 132.31 | 52.56 | 64.47 | 0.00 | 40.60 | 58.21 |
| | GH125 | 190.25 | 44.45 | 197.64 | 103.48 | 52.49 | 237.14 |
| | GH126 | 0.00 | 0.00 | 0.00 | 0.00 | 0.00 | 0.00 |
| | GH127 | 212.53 | 217.75 | 208.24 | 727.84 | 35.35 | 210.66 |
| | GH128 | 0.00 | 0.00 | 0.00 | 0.00 | 0.00 | 0.00 |
| | GH129 | 18.62 | 16.72 | 6.74 | 0.00 | 4.61 | 12.28 |
| | GH13 | 540.14 | 409.69 | 554.73 | 1272.31 | 439.85 | 568.96 |
| | GH1311 | 80.92 | 106.71 | 123.70 | 93.53 | 54.39 | 111.39 |
| | GH1312 | 0.00 | 0.00 | 0.00 | 0.00 | 0.00 | 0.00 |
| | GH1314 | 174.34 | 200.73 | 124.35 | 27.11 | 262.39 | 175.61 |
| | GH1318 | 27.95 | 43.15 | 36.79 | 16.89 | 26.64 | 22.58 |
| | GH1319 | 15.91 | 7.04 | 36.12 | 0.00 | 9.05 | 7.14 |
| | GH132 | 0.00 | 0.00 | 0.00 | 0.00 | 0.00 | 0.00 |
| | GH1320 | 36.42 | 77.38 | 81.19 | 49.95 | 28.82 | 52.04 |
| | GH1328 | 41.68 | 5.04 | 51.19 | 7.72 | 27.40 | 19.22 |
| | GH1329 | 28.18 | 3.12 | 19.82 | 0.00 | 23.51 | 4.30 |
| | GH133 | 0.00 | 3.37 | 0.00 | 0.00 | 6.45 | 6.38 |
| | GH1330 | 0.00 | 0.00 | 0.00 | 0.00 | 9.15 | 12.20 |
| | GH1331 | 108.27 | 208.27 | 201.54 | 169.76 | 70.28 | 145.12 |
| | GH1332 | 0.00 | 0.00 | 0.00 | 0.00 | 6.03 | 6.01 |
| | GH1336 | 22.58 | 42.58 | 65.77 | 23.42 | 18.28 | 55.62 |
| | GH1337 | 0.00 | 0.00 | 0.00 | 0.00 | 0.00 | 0.00 |
| | GH1338 | 105.13 | 46.86 | 82.53 | 194.11 | 48.66 | 91.37 |
| | GH1339 | 66.60 | 151.35 | 54.53 | 22.63 | 150.85 | 87.50 |
| | GH134 | 49.50 | 90.33 | 18.64 | 43.64 | 111.81 | 45.97 |
| | GH1341 | 16.68 | 42.76 | 33.29 | 23.29 | 12.85 | 18.29 |
| | GH1342 | 0.00 | 0.00 | 0.00 | 0.00 | 0.00 | 0.00 |
| | GH135 | 18.64 | 19.00 | 23.69 | 0.00 | 18.83 | 17.19 |
| | GH138 | 98.73 | 38.62 | 73.77 | 181.10 | 43.72 | 58.86 |
| | GH139 | 214.17 | 310.85 | 281.70 | 140.83 | 487.29 | 279.03 |
| | GH130 | 300.05 | 149.42 | 194.25 | 314.49 | 220.55 | 337.47 |
| | GH133 | 151.70 | 53.47 | 91.86 | 219.80 | 69.67 | 102.17 |
| | GH136 | 39.40 | 44.73 | 169.00 | 14.87 | 34.56 | 103.64 |
| | GH137 | 34.57 | 46.24 | 13.97 | 0.00 | 0.00 | 32.11 |
| | GH138 | 36.31 | 44.84 | 57.57 | 0.00 | 0.00 | 30.21 |
| | GH139 | 32.27 | 3.99 | 12.01 | 0.00 | 0.00 | 35.37 |
| | GH140 | 95.21 | 55.89 | 34.41 | 0.00 | 6.75 | 73.93 |
| | GH141 | 88.89 | 51.98 | 30.76 | 219.78 | 30.38 | 77.41 |
| | GH142 | 47.82 | 4.81 | 23.43 | 0.00 | 3.38 | 34.79 |
| | GH143 | 45.50 | 0.00 | 20.64 | 0.00 | 0.00 | 46.36 |
| | GH144 | 165.51 | 12.57 | 143.51 | 0.00 | 14.52 | 168.37 |
| | GH145 | 80.02 | 5.49 | 53.32 | 0.00 | 12.72 | 87.25 |
| | GH146 | 207.82 | 39.58 | 145.75 | 222.22 | 34.95 | 156.18 |
| | GH147 | 9.58 | 0.00 | 0.00 | 0.00 | 0.00 | 15.39 |
| | GH148 | 7.81 | 0.00 | 0.00 | 0.00 | 0.00 | 9.78 |
| | GH15 | 37.34 | 17.47 | 45.25 | 0.00 | 9.96 | 56.79 |
| | GH151 | 8.21 | 0.00 | 0.00 | 3.58 | 0.00 | 4.60 |
| | GH153 | 0.00 | 0.00 | 0.00 | 0.00 | 0.00 | 0.00 |
| | GH154 | 181.32 | 29.79 | 197.64 | 245.89 | 38.23 | 145.17 |
| | GH155 | 126.21 | 0.00 | 24.42 | 0.00 | 0.00 | 58.87 |
| | GH156 | 8.12 | 0.00 | 0.00 | 0.00 | 0.00 | 4.92 |
| | GH157 | 0.00 | 0.00 | 0.00 | 0.00 | 0.00 | 0.00 |
| | GH158 | 25.35 | 0.00 | 18.78 | 0.00 | 8.14 | 31.21 |
| | GH159 | 7.22 | 0.00 | 3.45 | 0.00 | 3.26 | 0.00 |
| | GH16 | 275.14 | 147.81 | 120.40 | 418.51 | 153.31 | 202.23 |
| | GH161 | 6.00 | 0.00 | 5.64 | 0.00 | 0.00 | 0.00 |
| | GH17 | 0.00 | 19.04 | 0.00 | 0.00 | 15.09 | 0.00 |
| | GH18 | 444.51 | 133.53 | 348.30 | 117.94 | 147.65 | 412.50 |
| | GH19 | 0.00 | 0.00 | 0.00 | 0.00 | 0.00 | 0.00 |
| | GH2 | 2262.78 | 1557.37 | 1758.71 | 2140.81 | 1407.11 | 2233.45 |
| | GH20 | 1070.37 | 378.05 | 528.65 | 487.80 | 397.64 | 794.23 |
| | GH23 | 737.48 | 576.70 | 617.55 | 622.60 | 517.84 | 575.09 |
| | GH24 | 436.29 | 83.93 | 219.39 | 24.53 | 158.78 | 174.90 |
| | GH25 | 345.26 | 747.04 | 372.34 | 844.08 | 640.12 | 475.95 |
| | GH26 | 121.50 | 144.85 | 166.88 | 196.83 | 171.32 | 282.58 |
| | GH27 | 219.70 | 115.42 | 103.91 | 16.36 | 82.67 | 268.83 |
| | GH28 | 890.71 | 477.03 | 713.67 | 1121.39 | 276.61 | 791.65 |
| | GH29 | 665.58 | 213.20 | 467.86 | 271.17 | 132.79 | 554.52 |
| | GH3 | 1449.81 | 900.10 | 1684.99 | 1667.86 | 1017.73 | 1505.0 |
| | GH30 | 0.00 | 0.00 | 4.14 | 0.00 | 0.00 | 0.00 |
| | GH301 | 3.59 | 0.00 | 0.00 | 0.00 | 5.89 | 0.00 |
| | GH302 | 5.24 | 0.00 | 24.12 | 0.00 | 5.63 | 20.85 |
| | GH303 | 34.34 | 27.89 | 28.34 | 196.92 | 7.68 | 43.52 |
| | GH304 | 83.63 | 57.71 | 64.56 | 208.07 | 9.43 | 145.17 |
| | GH305 | 0.00 | 0.00 | 6.58 | 0.00 | 0.00 | 0.00 |
| | GH306 | 57.19 | 0.00 | 19.95 | 0.00 | 0.00 | 39.89 |
| | GH308 | 0.00 | 5.82 | 13.26 | 0.00 | 4.44 | 6.69 |
| | GH31 | 583.42 | 438.06 | 551.37 | 579.37 | 390.20 | 622.59 |
| | GH32 | 251.27 | 372.34 | 367.36 | 405.40 | 278.17 | 338.46 |
| | GH33 | 351.26 | 164.19 | 123.00 | 200.57 | 179.15 | 246.36 |
| | GH35 | 173.81 | 96.47 | 210.41 | 440.73 | 69.31 | 148.54 |
| | GH36 | 347.44 | 402.76 | 308.40 | 550.89 | 325.90 | 398.31 |
| | GH37 | 0.00 | 0.00 | 0.00 | 0.00 | 0.00 | 0.00 |
| | GH38 | 108.50 | 71.00 | 149.85 | 4.38 | 91.77 | 245.94 |
| | GH39 | 47.64 | 25.08 | 9.95 | 20.70 | 68.12 | 62.34 |
| | GH4 | 90.30 | 88.13 | 79.67 | 74.52 | 36.00 | 37.86 |
| | GH42 | 190.09 | 125.42 | 192.45 | 45.88 | 72.99 | 162.58 |
| | GH43 | 52.13 | 0.00 | 5.08 | 10.74 | 0.00 | 65.65 |
| | GH431 | 73.80 | 49.14 | 56.87 | 226.83 | 0.00 | 43.03 |
| | GH4310 | 318.27 | 247.23 | 314.30 | 918.01 | 102.85 | 266.60 |
| | GH4311 | 30.10 | 0.00 | 30.30 | 8.80 | 34.93 | 29.46 |
| | GH4312 | 187.63 | 95.45 | 266.00 | 257.96 | 82.26 | 238.27 |
| | GH4316 | 30.10 | 4.14 | 69.59 | 0.00 | 20.99 | 77.13 |
| | GH4317 | 40.79 | 25.00 | 28.46 | 0.00 | 8.73 | 49.34 |
| | GH4318 | 88.10 | 9.78 | 45.61 | 0.00 | 0.00 | 83.86 |
| | GH4319 | 133.86 | 61.15 | 90.96 | 218.08 | 39.83 | 135.43 |
| | GH432 | 52.19 | 46.08 | 95.09 | 228.75 | 6.87 | 4.22 |
| | GH4322 | 5.67 | 0.00 | 0.00 | 0.00 | 6.68 | 19.58 |
| | GH4324 | 182.89 | 23.59 | 200.48 | 248.19 | 41.37 | 166.84 |
| | GH4326 | 60.26 | 49.66 | 77.04 | 21.27 | 30.21 | 50.31 |
| | GH4327 | 0.00 | 0.00 | 0.00 | 0.00 | 7.57 | 21.25 |
| | GH4328 | 98.68 | 28.32 | 145.80 | 0.00 | 52.37 | 91.00 |
| | GH4329 | 152.82 | 88.05 | 266.70 | 486.10 | 29.23 | 44.27 |
| | GH433 | 54.14 | 0.00 | 62.33 | 0.00 | 14.27 | 77.36 |
| | GH4330 | 8.17 | 0.00 | 0.00 | 0.00 | 0.00 | 0.00 |
| | GH4331 | 107.45 | 0.00 | 131.03 | 0.00 | 45.59 | 184.79 |
| | GH4332 | 0.00 | 0.00 | 0.00 | 0.00 | 0.00 | 0.00 |
| | GH4333 | 38.12 | 0.00 | 27.79 | 0.00 | 9.56 | 34.19 |
| | GH4334 | 191.22 | 51.27 | 111.73 | 228.14 | 32.12 | 203.24 |
| | GH4335 | 47.83 | 70.23 | 103.08 | 7.61 | 90.06 | 132.68 |
| | GH4337 | 0.00 | 0.00 | 0.00 | 0.00 | 0.00 | 0.00 |
| | GH434 | 146.28 | 98.14 | 188.99 | 500.66 | 18.17 | 121.80 |
| | GH435 | 0.00 | 0.00 | 3.65 | 197.05 | 7.54 | 0.00 |
| | GH437 | 47.99 | 36.59 | 40.18 | 193.74 | 0.00 | 0.00 |
| | GH438 | 0.00 | 0.00 | 5.51 | 0.00 | 0.00 | 0.00 |
| | GH439 | 77.62 | 0.00 | 13.87 | 0.00 | 0.00 | 67.32 |
| | GH44 | 0.00 | 17.11 | 12.16 | 0.00 | 0.00 | 23.72 |
| | GH48 | 0.00 | 0.00 | 15.67 | 0.00 | 0.00 | 14.90 |
| | GH5 | 6.49 | 88.14 | 34.67 | 0.00 | 94.71 | 14.99 |
| | GH51 | 0.00 | 40.83 | 24.17 | 0.00 | 72.79 | 24.24 |
| | GH513 | 0.00 | 0.00 | 0.00 | 0.00 | 0.00 | 5.56 |
| | GH518 | 0.00 | 0.00 | 0.00 | 0.00 | 0.00 | 0.00 |
| | GH52 | 52.55 | 98.01 | 87.03 | 19.44 | 124.86 | 102.42 |
| | GH521 | 19.08 | 47.08 | 41.41 | 443.75 | 0.00 | 0.00 |
| | GH522 | 0.00 | 0.00 | 0.00 | 0.00 | 0.00 | 0.00 |
| | GH525 | 0.00 | 0.00 | 12.25 | 0.00 | 0.00 | 0.00 |
| | GH526 | 25.30 | 0.00 | 0.00 | 0.00 | 0.00 | 0.00 |
| | GH527 | 0.00 | 0.00 | 0.00 | 0.00 | 0.00 | 0.00 |
| | GH537 | 0.00 | 16.51 | 14.15 | 0.00 | 40.55 | 17.15 |
| | GH538 | 0.00 | 0.00 | 0.00 | 0.00 | 0.00 | 0.00 |
| | GH54 | 91.56 | 213.85 | 134.57 | 685.98 | 172.63 | 180.81 |
| | GH544 | 0.00 | 0.00 | 0.00 | 0.00 | 17.39 | 15.17 |
| | GH546 | 30.28 | 0.00 | 34.92 | 0.00 | 9.53 | 34.91 |
| | GH55 | 24.83 | 3.38 | 0.00 | 0.00 | 7.46 | 20.85 |
| | GH57 | 27.91 | 55.36 | 44.51 | 182.60 | 53.57 | 61.44 |
| | GH58 | 0.00 | 19.00 | 0.00 | 0.00 | 42.72 | 21.02 |
| | GH50 | 9.82 | 3.65 | 36.54 | 0.00 | 0.00 | 17.99 |
| | GH51 | 358.77 | 286.39 | 433.11 | 942.06 | 159.16 | 345.66 |
| | GH53 | 123.35 | 119.97 | 131.25 | 309.15 | 91.43 | 169.32 |
| | GH55 | 51.25 | 0.00 | 79.03 | 0.00 | 26.17 | 106.37 |
| | GH57 | 126.37 | 55.31 | 92.84 | 182.74 | 61.13 | 75.61 |
| | GH59 | 7.52 | 18.43 | 10.29 | 0.00 | 49.74 | 22.74 |
| | GH63 | 207.22 | 67.06 | 150.33 | 10.19 | 48.91 | 143.38 |
| | GH64 | 0.00 | 0.00 | 0.00 | 0.00 | 0.00 | 1.56 |
| | GH65 | 100.12 | 16.43 | 107.70 | 4.94 | 8.54 | 69.58 |
| | GH66 | 38.52 | 56.22 | 28.83 | 0.00 | 7.20 | 39.89 |
| | GH67 | 104.91 | 43.82 | 97.14 | 236.86 | 17.64 | 41.77 |
| | GH68 | 0.00 | 0.00 | 0.00 | 0.00 | 0.00 | 0.00 |
| | GH70 | 0.00 | 0.00 | 0.00 | 0.00 | 0.00 | 0.00 |
| | GH73 | 419.55 | 338.44 | 386.72 | 763.84 | 242.17 | 185.38 |
| | GH74 | 124.61 | 37.05 | 38.65 | 0.00 | 98.98 | 81.62 |
| | GH76 | 192.34 | 0.00 | 83.02 | 0.00 | 34.68 | 177.46 |
| | GH77 | 333.62 | 415.17 | 298.77 | 366.91 | 448.02 | 391.33 |
| | GH78 | 387.76 | 199.50 | 250.13 | 247.18 | 104.64 | 444.14 |
| | GH79 | 0.00 | 0.00 | 8.21 | 0.00 | 0.00 | 0.00 |
| | GH8 | 83.25 | 97.84 | 180.39 | 246.51 | 50.59 | 47.47 |
| | GH81 | 0.00 | 0.00 | 0.00 | 0.00 | 0.00 | 0.00 |
| | GH84 | 181.09 | 4.34 | 73.25 | 239.94 | 8.49 | 81.24 |
| | GH85 | 10.90 | 0.00 | 0.00 | 9.50 | 0.00 | 34.41 |
| | GH87 | 0.00 | 0.00 | 0.00 | 0.00 | 0.00 | 0.00 |
| | GH88 | 423.12 | 52.18 | 471.98 | 3.00 | 75.53 | 303.48 |
| | GH89 | 203.89 | 35.65 | 48.26 | 0.00 | 5.71 | 133.67 |
| | GH9 | 168.45 | 35.55 | 267.35 | 6.17 | 52.30 | 323.80 |
| | GH91 | 37.57 | 3.66 | 0.00 | 0.00 | 0.00 | 14.85 |
| | GH92 | 1018.07 | 153.52 | 514.19 | 147.71 | 267.80 | 907.34 |
| | GH93 | 0.00 | 0.00 | 0.00 | 0.00 | 0.00 | 0.00 |
| | GH94 | 132.02 | 393.98 | 307.07 | 345.70 | 325.61 | 295.05 |
| | GH95 | 424.15 | 208.63 | 306.81 | 470.07 | 195.80 | 280.37 |
| | GH97 | 888.92 | 344.26 | 685.97 | 983.74 | 188.12 | 770.04 |
| | GH98 | 6.43 | 23.12 | 42.03 | 0.00 | 0.00 | 12.09 |
| | GH99 | 5.22 | 0.00 | 4.62 | 0.00 | 0.00 | 21.51 |
| | PL1 | 224.76 | 141.17 | 186.00 | 222.38 | 127.14 | 139.84 |
| | PL10 | 112.97 | 0.00 | 88.46 | 0.00 | 5.82 | 68.16 |
| | PL11 | 154.92 | 22.77 | 57.37 | 0.00 | 24.91 | 148.86 |
| | PL12 | 68.33 | 4.08 | 175.99 | 4.84 | 36.40 | 39.59 |
| | PL13 | 25.28 | 0.00 | 97.41 | 0.00 | 0.00 | 5.24 |
| | PL15 | 23.80 | 0.00 | 266.82 | 0.00 | 0.00 | 35.29 |
| | PL16 | 0.00 | 5.63 | 0.00 | 0.00 | 0.00 | 0.00 |
| | PL17 | 0.00 | 0.00 | 80.48 | 0.00 | 7.27 | 21.46 |
| | PL21 | 39.36 | 0.00 | 80.56 | 0.00 | 0.00 | 0.00 |
| | PL22 | 35.48 | 4.81 | 49.89 | 0.00 | 0.00 | 25.44 |
| | PL26 | 36.42 | 5.17 | 22.53 | 0.00 | 9.17 | 39.21 |
| | PL27 | 40.55 | 0.00 | 23.75 | 7.85 | 0.00 | 46.78 |
| | PL29 | 9.92 | 0.00 | 0.00 | 0.00 | 7.15 | 8.31 |
| | PL30 | 16.49 | 0.00 | 17.72 | 0.00 | 7.14 | 36.90 |
| | PL31 | 0.00 | 3.49 | 21.17 | 0.00 | 0.00 | 9.61 |
| | PL33 | 78.14 | 20.04 | 69.30 | 0.00 | 29.34 | 40.34 |
| | PL34 | 0.00 | 0.00 | 0.00 | 0.00 | 3.99 | 36.59 |
| | PL35 | 20.09 | 5.56 | 60.64 | 0.00 | 0.00 | 20.59 |
| | PL4 | 0.00 | 0.00 | 13.25 | 0.00 | 0.00 | 0.00 |
| | PL6 | 0.00 | 3.49 | 39.58 | 0.00 | 0.00 | 8.14 |
| | PL7 | 0.00 | 0.00 | 0.00 | 0.00 | 0.00 | 17.81 |
| | PL8 | 161.83 | 0.00 | 125.01 | 0.00 | 45.14 | 50.14 |
| | PL9 | 52.95 | 6.4 | 150.60 | 0.00 | 136.00 | 84.84 |

TABLE S8E
Spearman correlations of abundances of GH and PL
genes and levels of N-methylserotonin in fecal samples
collected from study participants at week 5.

N-methylserotonin

| CAZyme | Spearman Rho | P value | P adjusted (FDR) |
|---|---|---|---|
| PL9 | 0.505 | <0.001 | 0.025 |
| GH5_37 | 0.438 | 0.003 | 0.084 |
| GH5_8 | 0.415 | 0.005 | 0.090 |
| GH59 | 0.522 | <0.001 | 0.020 |
| GH30_5 | 0.435 | 0.003 | 0.084 |
| GH26 | 0.424 | 0.004 | 0.088 |
| GH5_4 | 0.538 | <0.001 | 0.018 |
| GH25 | 0.537 | <0.001 | 0.018 |
| GH13_31 | −0.438 | 0.003 | 0.084 |
| GH123 | −0.417 | 0.005 | 0.090 |
| GH13_19 | −0.430 | 0.004 | 0.084 |
| GH13_28 | −0.488 | 0.001 | 0.033 |
| GH24 | −0.393 | 0.008 | 0.136 |
| GH5_1 | 0.386 | 0.010 | 0.146 |
| GH110 | −0.378 | 0.011 | 0.157 |
| GH13_20 | −0.371 | 0.013 | 0.157 |
| GH151 | −0.371 | 0.013 | 0.157 |
| GH5_2 | 0.374 | 0.012 | 0.157 |
| GH148 | −0.359 | 0.017 | 0.187 |
| GH113 | 0.356 | 0.018 | 0.188 |
| GH5_7 | 0.339 | 0.024 | 0.245 |
| GH33 | −0.331 | 0.028 | 0.274 |
| GH23 | −0.314 | 0.038 | 0.343 |
| GH77 | 0.313 | 0.039 | 0.343 |
| PL35 | −0.310 | 0.041 | 0.345 |
| GH74 | 0.307 | 0.043 | 0.349 |
| GH13_8 | −0.303 | 0.046 | 0.358 |
| GH57 | −0.301 | 0.047 | 0.358 |
| GH13 | 0.296 | 0.051 | 0.377 |
| GH29 | −0.291 | 0.055 | 0.391 |
| GH13_14 | −0.280 | 0.066 | 0.402 |
| GH13_29 | −0.284 | 0.062 | 0.402 |
| GH13_9 | 0.279 | 0.067 | 0.402 |
| GH87 | −0.287 | 0.059 | 0.402 |
| GH94 | 0.278 | 0.068 | 0.402 |
| PL1 | 0.278 | 0.068 | 0.402 |
| GH10 | 0.261 | 0.087 | 0.414 |
| GH101 | −0.270 | 0.077 | 0.414 |
| GH103 | −0.259 | 0.089 | 0.414 |
| GH13_36 | −0.259 | 0.089 | 0.414 |
| GH19 | −0.260 | 0.088 | 0.414 |
| GH35 | −0.264 | 0.084 | 0.414 |
| GH38 | −0.263 | 0.084 | 0.414 |
| GH43_5 | 0.264 | 0.084 | 0.414 |
| GH5_18 | 0.272 | 0.074 | 0.414 |
| GH73 | −0.265 | 0.083 | 0.414 |
| GH13_30 | 0.251 | 0.100 | 0.449 |
| GH89 | −0.250 | 0.101 | 0.449 |
| GH136 | −0.244 | 0.111 | 0.481 |
| GH13_3 | 0.239 | 0.119 | 0.486 |
| GH20 | −0.237 | 0.121 | 0.486 |
| GH43_16 | 0.239 | 0.119 | 0.486 |
| GH55 | −0.238 | 0.119 | 0.486 |
| GH126 | −0.231 | 0.131 | 0.516 |
| GH5_44 | 0.228 | 0.137 | 0.529 |
| GH63 | −0.224 | 0.144 | 0.547 |
| GH102 | −0.212 | 0.167 | 0.582 |
| GH130 | 0.212 | 0.167 | 0.582 |
| GH133 | −0.216 | 0.159 | 0.582 |
| GH42 | −0.211 | 0.169 | 0.582 |
| GH66 | −0.211 | 0.170 | 0.582 |
| PL12 | −0.214 | 0.163 | 0.582 |
| GH32 | −0.205 | 0.182 | 0.598 |
| GH43_10 | 0.205 | 0.183 | 0.598 |
| PL6 | −0.205 | 0.182 | 0.598 |
| GH92 | −0.201 | 0.190 | 0.613 |
| GH85 | −0.193 | 0.208 | 0.662 |
| GH48 | 0.188 | 0.221 | 0.673 |
| GH84 | −0.190 | 0.217 | 0.673 |
| GH91 | −0.189 | 0.218 | 0.673 |
| GH109 | −0.186 | 0.226 | 0.676 |
| GH78 | −0.185 | 0.229 | 0.676 |
| GH18 | −0.183 | 0.233 | 0.681 |
| GH98 | −0.182 | 0.238 | 0.684 |
| GH5_22 | 0.180 | 0.241 | 0.685 |
| GH121 | 0.168 | 0.276 | 0.692 |
| GH128 | −0.173 | 0.262 | 0.692 |
| GH13_18 | −0.170 | 0.269 | 0.692 |
| GH147 | −0.168 | 0.275 | 0.692 |
| GH155 | −0.174 | 0.259 | 0.692 |
| GH4 | −0.175 | 0.256 | 0.692 |
| GH43_17 | −0.175 | 0.256 | 0.692 |
| GH43_33 | −0.168 | 0.275 | 0.692 |
| GH88 | −0.170 | 0.270 | 0.692 |
| PL11 | 0.169 | 0.274 | 0.692 |
| GH116 | −0.155 | 0.315 | 0.696 |
| GH13_32 | 0.150 | 0.330 | 0.696 |
| GH13_37 | 0.156 | 0.312 | 0.696 |
| GH144 | −0.150 | 0.330 | 0.696 |
| GH30_8 | 0.160 | 0.298 | 0.696 |
| GH31 | −0.164 | 0.288 | 0.696 |
| GH43_35 | 0.156 | 0.312 | 0.696 |
| GH5_38 | −0.160 | 0.301 | 0.696 |
| GH5_46 | −0.161 | 0.296 | 0.696 |
| GH50 | −0.153 | 0.323 | 0.696 |
| GH65 | −0.160 | 0.300 | 0.696 |
| GH95 | −0.157 | 0.308 | 0.696 |
| PL13 | −0.152 | 0.325 | 0.696 |
| PL31 | 0.157 | 0.308 | 0.696 |
| PL33 | −0.154 | 0.318 | 0.696 |
| PL34 | 0.152 | 0.324 | 0.696 |
| GH13_39 | −0.149 | 0.334 | 0.697 |
| PL8 | −0.147 | 0.340 | 0.703 |
| GH28 | 0.145 | 0.347 | 0.703 |
| GH44 | 0.146 | 0.344 | 0.703 |
| GH142 | −0.139 | 0.370 | 0.722 |
| GH9 | 0.139 | 0.367 | 0.722 |
| PL15 | −0.141 | 0.362 | 0.722 |
| PL22 | −0.138 | 0.373 | 0.722 |
| PL30 | −0.140 | 0.364 | 0.722 |
| GH120 | −0.131 | 0.396 | 0.754 |
| GH3 | −0.129 | 0.406 | 0.754 |
| GH36 | 0.128 | 0.407 | 0.754 |
| GH43_26 | −0.130 | 0.400 | 0.754 |
| GH76 | −0.130 | 0.401 | 0.754 |
| PL17 | −0.124 | 0.421 | 0.773 |
| GH97 | −0.123 | 0.426 | 0.776 |
| GH13_11 | −0.120 | 0.437 | 0.787 |
| GH43_12 | 0.120 | 0.440 | 0.787 |
| GH30_2 | −0.118 | 0.445 | 0.790 |
| GH16 | −0.112 | 0.470 | 0.823 |
| PL27 | −0.111 | 0.471 | 0.823 |
| GH13_41 | −0.110 | 0.478 | 0.828 |
| GH125 | −0.109 | 0.483 | 0.829 |
| GH111 | −0.087 | 0.573 | 0.835 |
| GH112 | −0.091 | 0.555 | 0.835 |
| GH129 | −0.097 | 0.533 | 0.835 |
| GH13_38 | −0.103 | 0.504 | 0.835 |
| GH140 | −0.096 | 0.537 | 0.835 |
| GH145 | −0.098 | 0.528 | 0.835 |
| GH15 | −0.084 | 0.588 | 0.835 |
| GH153 | −0.088 | 0.571 | 0.835 |
| GH158 | −0.107 | 0.491 | 0.835 |
| GH161 | −0.089 | 0.564 | 0.835 |
| GH2 | −0.091 | 0.559 | 0.835 |
| GH27 | −0.086 | 0.579 | 0.835 |
| GH30_4 | −0.092 | 0.552 | 0.835 |
| GH30_6 | −0.099 | 0.522 | 0.835 |
| GH39 | −0.100 | 0.517 | 0.835 |
| GH43_1 | 0.084 | 0.586 | 0.835 |
| GH43_18 | −0.103 | 0.505 | 0.835 |
| GH43_22 | 0.086 | 0.577 | 0.835 |
| GH43_29 | −0.090 | 0.563 | 0.835 |
| GH43_4 | 0.084 | 0.586 | 0.835 |
| GH43_8 | −0.100 | 0.517 | 0.835 |
| GH5_13 | −0.090 | 0.560 | 0.835 |
| GH93 | 0.092 | 0.551 | 0.835 |
| PL16 | −0.084 | 0.588 | 0.835 |
| PL21 | −0.097 | 0.533 | 0.835 |
| PL29 | −0.090 | 0.561 | 0.835 |
| GH51 | −0.079 | 0.612 | 0.864 |
| GH43_28 | −0.077 | 0.619 | 0.868 |
| GH117 | −0.075 | 0.628 | 0.875 |
| GH105 | 0.066 | 0.669 | 0.875 |
| GH138 | −0.068 | 0.659 | 0.875 |
| GH139 | −0.072 | 0.640 | 0.875 |
| GH141 | 0.071 | 0.647 | 0.875 |
| GH30 | 0.072 | 0.644 | 0.875 |
| GH43_2 | −0.068 | 0.663 | 0.875 |
| GH43_32 | 0.069 | 0.656 | 0.875 |
| GH43_9 | −0.068 | 0.660 | 0.875 |
| GH5 | 0.066 | 0.670 | 0.875 |
| PL4 | 0.072 | 0.645 | 0.875 |
| GH43_3 | −0.063 | 0.685 | 0.885 |
| GH79 | −0.063 | 0.686 | 0.885 |
| GH43_19 | 0.060 | 0.697 | 0.894 |
| GH43_31 | −0.057 | 0.714 | 0.910 |
| GH81 | −0.056 | 0.718 | 0.910 |
| PL26 | −0.050 | 0.747 | 0.941 |
| PL7 | 0.049 | 0.751 | 0.941 |
| GH108 | −0.032 | 0.834 | 0.941 |
| GH114 | 0.037 | 0.810 | 0.941 |
| GH115 | −0.032 | 0.835 | 0.941 |
| GH13_12 | −0.032 | 0.836 | 0.941 |
| GH13_2 | 0.029 | 0.853 | 0.941 |
| GH13_4 | 0.029 | 0.849 | 0.941 |
| GH13_42 | 0.034 | 0.827 | 0.941 |
| GH13_5 | 0.035 | 0.823 | 0.941 |
| GH137 | −0.038 | 0.806 | 0.941 |
| GH143 | 0.028 | 0.858 | 0.941 |
| GH154 | −0.035 | 0.822 | 0.941 |
| GH157 | 0.029 | 0.853 | 0.941 |
| GH159 | 0.046 | 0.766 | 0.941 |
| GH17 | 0.042 | 0.785 | 0.941 |
| GH37 | −0.045 | 0.770 | 0.941 |
| GH43 | −0.032 | 0.839 | 0.941 |
| GH43_24 | −0.033 | 0.833 | 0.941 |
| GH43_27 | 0.038 | 0.804 | 0.941 |
| GH43_37 | −0.045 | 0.774 | 0.941 |
| GH5_21 | −0.028 | 0.858 | 0.941 |
| GH5_26 | −0.029 | 0.851 | 0.941 |
| GH53 | −0.043 | 0.781 | 0.941 |
| GH64 | −0.027 | 0.862 | 0.941 |
| GH68 | −0.032 | 0.836 | 0.941 |
| GH70 | −0.032 | 0.836 | 0.941 |
| GH5_5 | −0.024 | 0.875 | 0.951 |
| GH8 | −0.022 | 0.886 | 0.958 |
| GH43_11 | 0.021 | 0.894 | 0.961 |
| GH5_27 | 0.020 | 0.898 | 0.961 |
| GH1 | −0.018 | 0.906 | 0.965 |
| GH43_7 | 0.017 | 0.914 | 0.968 |
| GH156 | −0.015 | 0.921 | 0.971 |
| GH43_30 | −0.014 | 0.929 | 0.974 |
| GH146 | −0.013 | 0.935 | 0.976 |
| GH43_34 | −0.010 | 0.950 | 0.978 |
| GH5_25 | 0.011 | 0.946 | 0.978 |
| GH99 | −0.011 | 0.942 | 0.978 |
| GH106 | 0.006 | 0.967 | 0.990 |
| GH127 | 0.005 | 0.972 | 0.991 |
| GH30_1 | 0.002 | 0.990 | 0.997 |
| GH30_3 | 0.001 | 0.996 | 0.997 |
| GH67 | −0.001 | 0.997 | 0.997 |
| PL10 | −0.001 | 0.994 | 0.997 |

S9. Assessment of commercially available orange fibers for the presence of N-methylserotonin.

| | | | | Citrus | Abundance |
|---|---|---|---|---|---|
| Fiber Type | Classification | Origin | Source | Type | (Mean ± SD) |
| Orange Fiber | Ground | USA | Fiberstar | Orange | N/A (Control) |
| Orange Peel | Ground | India | M. Naturelife | Orange | 67 ± 21% |
| Orange Peel | Ground | Germany | Herbafoods | Orange | 57 ± 5% |
| Lime Peel | Ground | China | Bixa | Sweet Lime | 53 ± 36% |
| Orange Peel | Whole | USA | FrontierCoop | Orange | 37 ± 22% |
| Orange Peel | Whole | USA | Spiceway | Orange | 31 ± 15% |
| Orange Peel | Ground | Unknown | XPRS Nutra | Orange | 30 ± 24% |
| Orange Peel | Ground | China | ECO-Taste | Orange | 22 ± 11% |
| Lime Granule | Granule | USA | Crimson&Clove | Sweet Lime | 21 ± 1% |
| Orange Peel | Whole | Spain? | McCormick | Valencia | 16 ± 5% |
| Orange Fiber (Fine) | Ground | USA | Fiberstar | Orange | 15 ± 8% |
| Lemon Peel | Whole | USA | Spiceway | “Californian” | 15 ± 2% |
| Orange Peel | Whole | India | YAMEES | Orange | 14 ± 6% |
| Key Lime Peel | Whole | USA | Brewer's best | Key Lime | 13 ± 2% |
| Lemon Granule | Granule | India | Naturevibe | Orange | 12 ± 6% |
| Orange Peel | Whole | USA | McCormick | Orange | 12 ± 5% |
| Lemon Peel | Whole | USA | McCormick | Lemon | 11 ± 4% |
| Lemon Peel Powder | Ground | India | Minature | Lemon | 10 ± 1% |
| Lemon Peel | Whole | India | YAMEES | Lemon | 9 ± 5% |
| Citrus Peel | Ground | Germany | Herbafoods | Citrus | 6 ± 1% |
| Prebiotic Mix | Ground | Unknown | Garden of Life | Unknown | 5% ± 1% |
| Lemon Peel | Ground | Spain | Fruidles | Lemon | 3 ± 0.3% |
| Citrus Fiber | Ground | Netherlands | Ceamsa | Citrus | Negligible |
| | | | | | (0.06 ± 0.01%) |
| Citrus Pectin | Ground | Netherlands | Ceamsa | Citrus | Negligible |
| | | | | | (0.05 ± 0.01%) |

TABLE S10
Assessment of locally commercially available citrus fruits for the presence of N-methylserotonin.

| | | | Enzyme | vs. Orange Fiber | Additional |
|---|---|---|---|---|---|
| Fiber Type | Source | Part | added? | (Mean ± SD) | Conditions/notes |
| Table Orange | St. Louis, | Skin | + | 161 ± 65% | |
| | MO | | | | |
| Table Orange | St. Louis, | Skin | | 1 ± 0.1% | MeOH only (no enzyme) |
| | MO | | | | |
| Table Orange | St. Louis, | Skin | | 3 ± 1% | Ground (no enzyme) |
| | MO | | | | |
| Table Orange | St. Louis, | Skin | | 1 ± 1% | N2 freeze-thaw (no enzyme) |
| | MO | | | | |
| Table Orange | St. Louis, | Fruit (With | + | 122 ± 52% | |
| | MO | Pulp) | | | |
| Table Orange | St. Louis, | Pulp | + | 35 ± 4% | |
| | MO | | | | |
| Table Orange (Hand- | St. Louis, | Juice | + | 36 ± 1% | |
| squeezed) | MO | | | | |
| Minute Maid OJ | USA | Juice (“no | + | 6 ± 4% | 100% Juice, pasteurized |
| | | pulp”) | | | |
| Valencia Orange OJ | USA | Juice (“no | + | 10 ± 5% | 100% Juice |
| | | pulp”) | | | |
| Organic Orange Juice | USA | Juice | + | Negligible | Filtered prior to |
| | | | | (~0.4 ± 0.5%) | processing (0.22 μm) |
| Table Orange (Hand- | St. Louis, | Juice | + | Negligible | Filtered prior to |
| squeezed) | MO | | | (~0.2 ± 0.01%) | processing (0.22 μm) |
| Table Lemon | St. Louis, | Skin | + | 6 ± 3% | |
| | MO | | | | |
| Table Lemon | St. Louis, | Fruit (With | + | 5 ± 2% | |
| | MO | Pulp) | | | |
| Table Lemon | St. Louis, | Pulp | + | 6 ± 1% | |
| | MO | | | | |
| Table Lemon | St. Louis, | Juice | + | 1 ± 1% | |
| | MO | | | | |
| Table Lime | St. Louis, | Skin | + | 5 ± 5% | |
| | MO | | | | |
| Table Lime | St. Louis, | Fruit (With | + | 2 ± 1% | |
| | MO | Pulp) | | | |
| Table Lime | St. Louis, | Pulp | + | 4 ± 1% | |
| | MO | | | | |
| Table Lime | St. Louis, | Juice | + | 1 ± 1% | |
| | MO | | | | |
| Grapefruit | St. Louis, | Skin | + | N/A | |
| | MO | | | | |
| Grapefruit | St. Louis, | Fruit (With | + | N/A | |
| | MO | Pulp) | | | |
| Grapefruit | St. Louis, | Juice | + | N/A | |
| | MO | | | | |
| Grapefruit | St. Louis, | Ground | + | N/A | |
| | MO | | | | |

TABLE S11A
N-Methylserotonin screening in cash crops
and staples relative to orange fiber (OF).

| Cash Crops & | | | % |
|---|---|---|---|
| Staples | Component | Country | OF |
| Sugarcane | Fiber | USA | — |
| Sugarcane | Whole | USA | — |
| Corn | Fiber | USA | — |
| Corn | Grain | USA | — |
| Wheat | Bran | USA | — |
| Wheat | Aleuron | USA | — |
| Wheat | Grain | USA | — |
| Rice | Fiber | USA | — |
| Rice | Grain (white) | USA | — |
| Rice | Grain (whole) | China | — |
| Soy | Fiber | Canada | — |
| Soy | Cotyledon | Switzerland | — |
| Soy | Beans | USA | — |
| Barley | Fiber | USA | — |
| Barley | Grain | USA | — |
| Sorghum | Grain | China | — |
| Millet | Grain | China | — |
| Potato | Fiber | Netherlands | — |
| Potato | Tuber | USA | — |
| Cassava | Tuber | China | — |
| Sugar Beet | Root | USA | — |
| Chick Pea | Beans | USA | — |
| Oat | Fiber | Canada | — |
| Oat | Grain | USA | — |
| Yam | Tuber | USA | — |
| Sweet Potato | Tuber | USA | — |
| Plantain | Whole fruit | USA | — |
| Lentils | Beans | USA | — |
| Rye | Grain | USA | — |
| Buckwheat | Grain | Japan | — |
| Taro | Tuber | USA | — |

TABLE S11C
N-Methylserotonin screening in spices
relative to orange fiber (OF).

| Spices | Component | Country | % OF |
|---|---|---|---|
| Japanese Pepper | Pre-Grounded | Japan | 19 ± 2% |
| Sansho Pepper | Pre-Grounded | Japan | 43 ± 9% |
| Sichuan Peppercorn | Whole, dried | China | 26 ± 1% |
| Black Pepper | Pre-Grounded | USA | — |
| Yellow Mustard | Pre-Grounded | Canada | — |
| Horseradish | Root | Japan | — |
| Wasabi | Root | Japan | — |
| Black Sesame | Seed | China | — |
| White Sesame | Seed | China | — |
| Fennel | Seed | China | — |
| Clove | Dried bud | China | — |
| Cinnamon | Bark | China | — |
| Cinnamon | Oil | China | — |
| Cardamom | Dried fruit | China | — |
| Hawthorn | Dried fruit | China | — |
| Cumin | Pre-Grounded | China | — |
| Coriander | Leaves | USA | — |
| Marjoram | Dried leaves | UK | — |
| Bayleaf | Dried leaves | USA | — |
| Sage | Dried leaves | USA | — |
| Garlic | Whole bulb | USA | — |
| Ginger | Whole root | USA | — |
| Ginger | Pre-Grounded | USA | — |

TABLE S11B
N-Methylserotonin screening in common prebiotic
supplements relative to orange fiber (OF).

| | | |
|---|---|---|
| | | % |
| Common Prebiotic Supplements | — | OF |

| | | | |
|---|---|---|---|
| Cocao | Fiber | Switzerland | — |
| Acacia | Fiber | USA | — |
| Psyllium | Fiber | USA | — |
| Chia Seed | Fiber | USA | — |

TABLE S11D
N-Methylserotonin screening in fruits relative to orange fiber (OF).

| Fruits | Component | Country | % OF |
|---|---|---|---|
| Apple | Fiber | Germany | — |
| Apple | Whole fruit | USA | — |
| Tomato | Fiber | USA | — |
| Tomato | Whole fruit | USA | — |
| Peach | Whole fruit | USA | — |
| Pear | Whole fruit | USA | — |
| Cranberry | Whole fruit | USA | — |
| Black grapes | Whole fruit | USA | — |
| Green grapes | Whole fruit | USA | — |
| Cherry | Whole fruit | USA | — |
| Banana | Edible Fruit | USA | — |
| Strawberry | Whole fruit | USA | — |
| Blueberry | Whole fruit | USA | — |
| Blackberry | Whole fruit | USA | — |
| Raspberry | Whole fruit | USA | — |
| Kiwi | Edible Fruit | USA | — |
| Mango | Edible Fruit | USA | — |
| Pineapple | Edible Fruit | USA | — |
| Watermelon | Edible Fruit | USA | — |
| Watermelon | Skin | USA | — |
| Cantaloupe | Edible Fruit | USA | — |
| Cantaloupe | Skin | USA | — |
| Avocado | Fruit (no seed) | USA | — |
| Avocado | Seed | USA | — |
| Avocado | Skin | USA | — |
| Pomegranate | Edible Fruit | USA | — |
| Pomegranate | Skin | USA | — |
| Plum | Whole fruit | USA | — |

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined herein. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A synbiotic composition comprising at least one type of plant fiber and at least one microbial strain.

2. The synbiotic composition of claim 1, wherein the at least one type of plant fiber comprises a Rutaceae family plant fiber.

3. The synbiotic composition of claim 2, wherein the Rutaceae family plant fiber comprises citrus fiber.

4. The synbiotic composition of claim 3, wherein the citrus fiber comprises orange fiber.

5. The synbiotic composition of claim 1, wherein the at least one microbial strain comprises at least one bacterial strain.

6. The synbiotic composition of claim 5, wherein the at least one bacterial strain is selected from Bacteroides, Parabacteroides, Collinsella, and combinations thereof.

7. The synbiotic composition of claim 6, wherein the at least one bacterial strain comprises at least one strain of Bacteroides ovatus, Bacteroides finegoldii, Parabacteroides distasonis, Collinsella aerofaciens, or combinations thereof.

8. The synbiotic composition of claim 7, wherein the at least one bacterial strain comprises Bacteroides ovatus TSDC 17.2.

9. The synbiotic composition of claim 1, further comprising an iron-containing porphyrin.

10. The synbiotic composition of claim 9, wherein the iron-containing porphyrin is hemin.

11. A method for locally delivering a bioactive compound to a subject in need thereof, the method comprising:
administering to the subject a therapeutically effective amount of a synbiotic composition comprising at least one type of plant fiber and at least one microbial strain.

12. The method of claim 11, wherein the at least one microbial strain is a source of at least one CAZyme.

13. The method of claim 12, wherein the at least one CAZyme is selected from PL9, GH5_37, GH5_8, GH59, GH30_5, GH26, GH5_4, GH25, GH13_31, GH123, GH13_19, GH13_28, and combinations thereof.

14. The method of claim 13, wherein the at least one CAZyme comprises PL9.

15. The method of claim 11, wherein the bioactive compound is N-methylserotonin.

16-41. (canceled)

42. The method of claim 11, wherein the at least one type of plant fiber comprises orange fiber.

43. The method of claim 11, wherein the at least one microbial strain comprises at least one bacterial strain selected from Bacteroides, Parabacteroides, Collinsella, and combinations thereof.

44. The method of claim 43, wherein the at least one bacterial strain comprises Bacteroides ovatus TSDC 17.2.

45. The method of claim 11, wherein the synbiotic composition further comprises an iron-containing porphyrin.

46. The method of claim 45, wherein the iron-containing porphyrin is hemin.


SEQUENCE LISTING (2 sequences)

SEQ ID NO: 1 - DNA, 58 bp, synthetic construct
  source 1..58: mol_type = other DNA
aatgatacggcgaccaccgagatctacactctttccctacacgacgctcttccgatct

SEQ ID NO: 2 - DNA, 61 bp, synthetic construct
  source 1..61: mol_type = other DNA
caagcagaagacggcatacgagatcggtctcggcattcctgctgaaccgctcttccgatct